(19) United States
(12) Patent Application Publication
(10) Pub. No.: US 2026/0049092 A1
(43) Pub. Date: Feb. 19, 2026

(54) ANTIBACTERIAL COMPOUNDS

(71) Applicant: Blacksmith Medicines, Inc., San Diego, CA (US)

(72) Inventors: David LONERGAN, San Diego, CA (US); David Pifer MARTIN, San Diego, CA (US); Junhua FAN, San Diego, CA (US); Min TENG, San Diego, CA (US); Baskar NAMMALWAR, San Diego, CA (US); David T. PUERTA, Rancho Santa Fe, CA (US)

(21) Appl. No.: 19/102,043

(22) PCT Filed: Aug. 8, 2023
(86) PCT No.: PCT/US2023/071877

§ 371 Date: Feb. 7, 2025

Related U.S. Application Data
(60) Provisional application No. 63/396,895, filed on Aug. 10, 2022.

(51) Int. Cl. C07D 491/107; A61K 31/4174; A61K 31/4178; A61K 31/4439; A61K 31/454; A61K 31/5377; A61K 31/538; A61P 31/04; C07D 233/64; C07D 401/10; C07D 401/12; C07D 403/10; C07D 403/12; C07D 403/14; C07D 405/10; C07D 405/12; C07D 405/14; C07D 409/12; C07D 413/10; C07D 413/14; C07D 493/04

(52) U.S. Cl. CPC C07D 491/107; A61K 31/4174; A61K 31/4178; A61K 31/4439; A61K 31/454; A61K 31/5377; A61K 31/538; A61P 31/04; C07D 233/64; C07D 401/10; C07D 401/12; C07D 403/10; C07D 403/12; C07D 403/14; C07D 405/10; C07D 405/12; C07D 405/14; C07D 409/12; C07D 413/10; C07D 413/14; C07D 493/04

(57) ABSTRACT

Provided herein are heterocyclic compounds and pharmaceutical compositions comprising said compounds that are useful for inhibiting the growth of gram-negative bacteria. The subject compounds and compositions are useful for the treatment of bacterial infections, such as pneumonia.

### CROSS-REFERENCE TO RELATED APPLICATIONS

This application claims the benefit of U.S. Provisional Patent Application No. 63/396,895, filed Aug. 10, 2022, which is incorporated herein by reference in its entirety.

### STATEMENT AS TO FEDERALLY SPONSORED RESEARCH

This invention was made with Government support under Agreement HHS0100201600038C, awarded by HHS. The Government has certain rights in the invention.

### BACKGROUND OF THE INVENTION

A need exists in the medicinal arts for the effective treatment of illness caused by bacterial infection.

### SUMMARY OF THE INVENTION

Provided herein are heterocyclic compounds and pharmaceutical compositions comprising said compounds that are useful for inhibiting the growth of gram-negative bacteria. The subject compounds and compositions are useful for the treatment of bacterial infection, such as pneumonia and the like. In some embodiments, compounds described herein are UDP-{3-O—[(R)-3-hydroxymyristoyl]}-N-acetylglucosamine deacetylase (LpxC) modulator compounds. In some embodiments, the compounds described herein are UDP-{3-O—[(R)-3-hydroxymyristoyl]}-N-acetylglucosamine deacetylase (LpxC) antagonists. In some embodiments, the compounds described herein are UDP-{3-O—[(R)-3-hydroxymyristoyl]}-N-acetylglucosamine deacetylase (LpxC) inhibitors.

In one aspect, the present disclosure provides a compound of Formula (I):

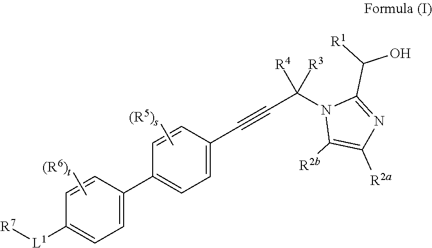

or a pharmaceutically acceptable salt, solvate, or stereoisomer thereof, wherein:
   R¹ is C₁-C₄ alkyl;
   R^{2a} and R^{2b} are each independently hydrogen, halogen, or C₁-C₄ alkyl;
   R³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂;
   R⁴ is hydrogen or C₁-C₄ alkyl;
   each R⁵ and R⁶ is independently halogen, or C₁-C₄ alkyl;
   L¹ is a bond, —(C₁-C₆ alkylene)-, —X¹—, or —X²—(C₁-C₆ alkylene)-, wherein:
      X¹ and X² are each selected from: —O—, —N(R⁹)—, —S—, —S(═O)—, —S(═O)₂—, and —S(═O)(═NR⁹)—, wherein:
         R⁹ is hydrogen or C₁-C₆ alkyl;
   R⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R¹⁰ groups, wherein:
      each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein:
         each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃;
         or two R⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo;
   s is 0, 1, or 2;
   t is 0, 1, or 2; and
   wherein at least one of the following:
      (i) s is 1 or 2, and at least one R⁵ is halogen;
      (ii) t is 1 or 2, and at least one R⁶ is halogen; and
      (iii) the alkyl, heteroalkyl, cycloalkyl, or heterocycloalkyl of R⁷ is substituted by 1, 2, or 3 R¹⁰ groups, and at least one R¹⁰ is halogen.

In some embodiments, at least one R⁵, R⁶, or R¹⁰ is —F. In some embodiments, at least one R⁵ or R⁶ is —F. In some embodiments, at least one R¹⁰ is —F. In some embodiments, at least two R⁵, R⁶, or R¹⁰ is —F. In some embodiments, at least two R⁵ or R⁶ is —F. In some embodiments, at least two R¹⁰ is —F.

In some embodiments, R¹ is —CH₃. In some embodiments, R^{2a} is hydrogen; and R^{2b} is hydrogen. In some embodiments, R⁴ is hydrogen. In some embodiments, each R⁵ is independently —F, —Cl, or —CH₃. In some embodiments, each R⁵ is —F. In some embodiments, each R⁶ is —F

In some embodiments, the compound is a compound of Formula (IIa):

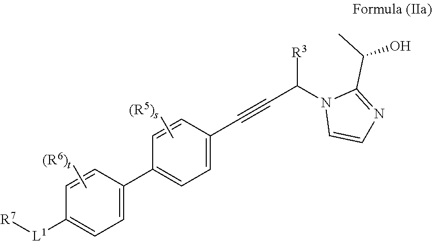

or a pharmaceutically acceptable salt, solvate, or stereoisomer thereof, wherein:
each R⁵ is —F; and
each R⁶ is —F.

In some embodiments, the compound is a compound of Formula (IIIa):

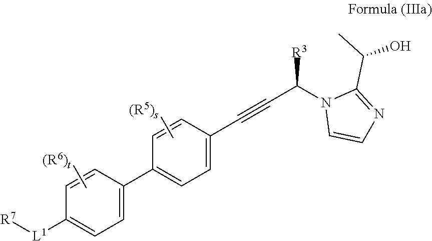

or a pharmaceutically acceptable salt, solvate, or stereoisomer thereof, wherein:
each R⁵ is —F; and
each R⁶ is —F.

In some embodiments, R³ is hydrogen or —(C₁-C₄ alkylene)-OH. In some embodiments, R³ is —(C₁-C₄ alkylene)-OH. In some embodiments, R³ is —CH₂OH. In some embodiments, R⁹ is hydrogen.

In some embodiments, L¹ is a bond, —(C₁-C₄ alkylene)-, —X¹—, or —X²—(C₁-C₄ alkylene)-, wherein: X¹ and X² are each selected from: —O—, —N(H)—, —S—, —S(═O)—, —S(═O)₂—, and —S(═O)(═NH)—. In some embodiments, L¹ is —X¹— or —X²—(C₁-C₄ alkylene)-, wherein: X¹ and X² are each selected from: —O—, —N(H)—, —S—, —S(═O)—, —S(═O)₂—, and —S(═O)(═NH)—.

In some embodiments, L¹ is —X¹— or —X²—(C₁-C₄ alkylene)-, wherein: X¹ and X² are each selected from: —O—. In some embodiments, L¹ is —O—.

In some embodiments, R⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R¹⁰ groups, wherein: each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃.

In some embodiments, R⁷ is 4- to 8-membered heterocycloalkyl, where the 4- to 8-membered heterocycloalkyl is unsubstituted or substituted by 1, 2, or 3 R¹⁰ groups, wherein: each R¹⁰ is independently selected from: halogen, —OR⁸, —N(R⁸)₂, —NHSO₂R⁸, —CH₂CN, C₁-C₄ alkyl, —C(═O)—C₁-C₄ hydroxyalkyl, C₁-C₄ hydroxyalkyl, and C₁-C₄ methoxyalkyl; and each R⁸ is independently hydrogen, C₁-C₄ alkyl, —C(═O)—C₁-C₄ alkyl, or 4- to 6-membered heterocycloalkyl, wherein the alkyl is unsubstituted or substituted by 1, 2, or 3 groups independently selected from —F, —CN, —OH, —CO₂H, —C(═NH)NH₂, and 5-membered monocyclic heteroaryl which is unsubstituted or substituted by 1 —CONH₂ group.

In some embodiments, R⁷ is 4- to 8-membered heterocycloalkyl, where the 4- to 8-membered heterocycloalkyl is unsubstituted or substituted by 1, 2, or 3 R¹⁰ groups, wherein: each R¹⁰ is independently selected from: halogen, —OH, —OMe, —N(R⁸)₂, —NHSO₂R⁸, and —CH₂CN; and each R⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl.

In some embodiments, R⁷ is 4- to 6-membered heterocycloalkyl, where the 4- to 6-membered heterocycloalkyl is unsubstituted or substituted by 1, 2, or 3 R¹⁰ groups, wherein: each R¹⁰ is independently selected from: —F, —OH, —OCH₃, and —NH₂.

In some embodiments, L¹-R⁷ is

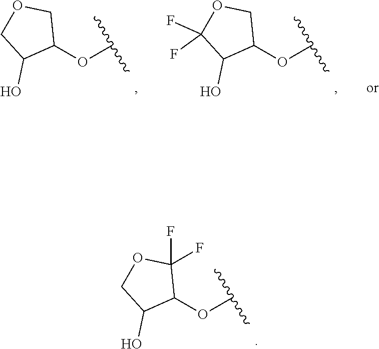 [SMILES: *OC1COCC1O ; *OC1COC(F)(F)C1O ; *OC1C(O)COC1(F)F]

In some embodiments, L¹-R⁷ is

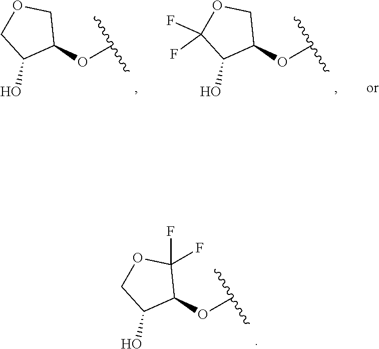 [SMILES: *O[C@@H]1COC[C@H]1O ; *O[C@@H]1COC(F)(F)[C@H]1O ; *O[C@H]1[C@H](O)COC1(F)F]

In some embodiments, R¹ is —CH₃; R^{2a} and R^{2b} are each hydrogen; R³ is hydrogen, —(C₁-C₄ alkylene)-OH, or —(C₁-C₄ alkylene)-NH₂; R⁴ is hydrogen; each R⁵ and R⁶ is halogen; L¹ is —X¹— or —X²—(C₁-C₄ alkylene)-, wherein: X¹ and X² are each selected from: —O—; and R⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R¹⁰ groups, wherein: each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃.

In some embodiments, R³ is hydrogen or —(C₁-C₄ alkylene)-OH; L¹ is —X¹— or —X²—(C₁-C₄ alkylene)-, wherein: X¹ and X² are each selected from: —O—; R⁷ is 4- to 8-membered heterocycloalkyl, where the 4- to 8-membered heterocycloalkyl is unsubstituted or substituted by 1, 2, or 3 R¹⁰ groups, wherein: each R¹⁰ is independently selected from: halogen, —OH, —OMe, —N(R⁸)₂, —NHSO₂R⁸, and —CH₂CN; and each R⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl.

In some embodiments, R³ is CH₂OH; L¹ is —O—; R⁷ is 4- to 6-membered heterocycloalkyl, where the 4- to 6-membered heterocycloalkyl is unsubstituted or substituted by 1, 2, or 3 R¹⁰ groups, wherein: each R¹⁰ is independently selected from: —F, —OH, —OCH₃, and —NH₂.

In some embodiments, the compound is a compound of Formula (IV):

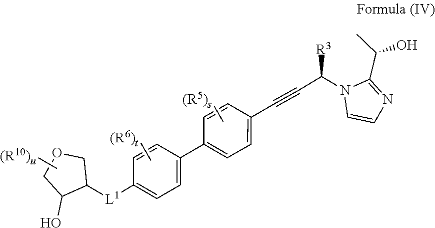

or a pharmaceutically acceptable salt, solvate, or stereoisomer thereof, wherein: R³ is —(C₁-C₄ alkylene)-OH, —(C₁-C₄ alkylene)-NH₂, —(C₃-C₆ cycloalkylene)-OH, or —(C₃-C₆ cycloalkylene)-NH₂; each R⁵ and R⁶ is independently —F, —Cl, or C₁-C₄ alkyl; L¹ is a —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and —S(═O)(═NR⁹)—, wherein: R⁹ is hydrogen or C₁-C₆ alkyl; each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen or C₁-C₄ alkyl s is 0, 1, or 2; t is 0, 1, or 2; and u is 0, 1, or 2

In some embodiments, the compound is a compound of Formula (V):

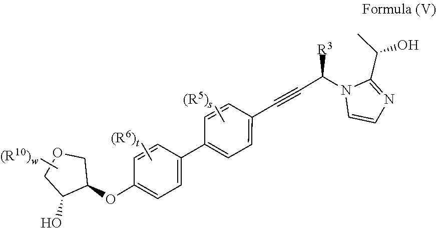

or a pharmaceutically acceptable salt, solvate, or stereoisomer thereof.

In some embodiments, s is 1, or 2; and at least one R⁵ is —F. In some embodiments, t is 1, or 2; and at least one R⁶ is —F. In some embodiments, u is 1, or 2; and at least one R¹⁰ is —F. In some embodiments, R³ is —(C₁-C₄ alkylene)-OH. In some embodiments, R³ is —CH₂OH.

In another aspect, the present disclosure provides a compound of Formula (VI):

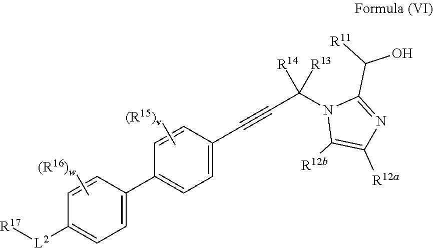

or a pharmaceutically acceptable salt, solvate, or stereoisomer thereof, wherein:
   R¹¹ is C₁-C₄ alkyl;
   R^{12a} and R^{12b} are each independently hydrogen, halogen, or C₁-C₄ alkyl;
   R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂;
   R¹⁴ is hydrogen or C₁-C₄ alkyl;
   each R¹⁵ and R¹⁶ is independently halogen, or C₁-C₄ alkyl;
   R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 groups independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; wherein:
      each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃;
      or two R¹⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo;
   L² is a bond, —(C₁-C₆ alkylene)-, —X³—, or —X⁴—(C₁-C₆ alkylene)-, wherein:
      X³ and X⁴ are each independently selected from: —O—, —N(R¹⁹)—, —S—, —S(═O)—, —S(═O)₂—, and —S(═O)(═NR¹⁹)—, wherein:
         R¹⁹ is hydrogen or C₁-C₆ alkyl;
      provided that when L² is a bond, —(C₁-C₆ alkylene)-, —O—, or —O—(C₁-C₆ alkylene)- then at least one of the following:
         (i) R¹³ is —(C₃-C₆ cycloalkylene)-OH or —(C₃-C₆ cycloalkylene)-NH₂;
         (ii) R¹⁴ is C₁-C₄ alkyl,
         (iii) R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂; or
         (iv) L² is —O—(C₁-C₆ alkylene)- and R¹⁷ is an unsubstituted or substituted 5-membered heterocycloalkyl;
   v is 0, 1, or 2; and
   w is 0, 1, or 2.

In some embodiments, R¹¹ is —CH₃. In some embodiments, R^{12a} is hydrogen; and R^{12b} is hydrogen. In some embodiments, R¹⁴ is hydrogen. In some embodiments, each R¹⁵ is independently —F, —Cl, or —CH₃. In some embodiments, each R¹⁵ is independently —F. In some embodiments, each R¹⁶ is independently —F, —Cl, or —CH₃. In some embodiments, each R¹⁶ is independently —F.

In some embodiments, the compound is a compound of Formula (VIIa):

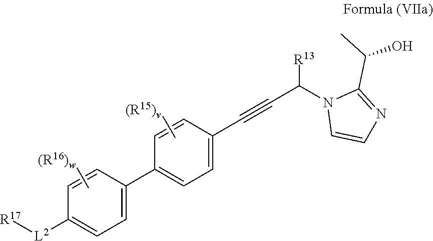

or a pharmaceutically acceptable salt, solvate, or stereoisomer thereof.

In some embodiments, the compound is a compound of Formula (VIIIa):

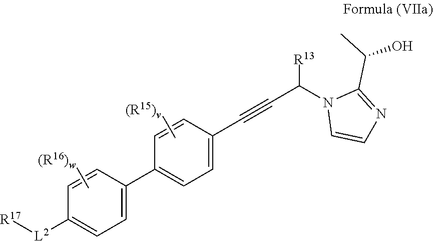

or a pharmaceutically acceptable salt, solvate, or stereoisomer thereof.

In some embodiments, v is 1 or 2. In some embodiments, v is 0. In some embodiments, w is 1 or 2. In some embodiments, w is 0. In some embodiments, L² is —X³— or —X⁴—(C₁-C₆ alkylene)-; and X³ and X⁴ are each independently selected from: —N(R¹⁹)—, —S—, —S(═O)—, —S(═O)₂—, and —S(═O)(═NR¹⁹). In some embodiments, R³ is hydrogen or —(C₁-C₄ alkylene)-OH. In some embodiments, R³ is —(C₁-C₄ alkylene)-OH. In some embodiments, R³ is —CH₂OH.

In some embodiments, R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 groups independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is un substituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃.

In some embodiments, R¹⁷ is 4- to 8-membered heterocycloalkyl, where the 4- to 8-membered heterocycloalkyl is unsubstituted or substituted by 1, 2, or 3 groups independently selected from: halogen, —OR¹⁸, —N(R¹⁸)₂, —NHSO₂R¹⁸, —CH₂CN, C₁-C₄ alkyl, —C(═O)—C₁-C₄ hydroxyalkyl, C₁-C₄ hydroxyalkyl, and C₁-C₄ methoxyalkyl; and each R¹⁸ is independently hydrogen, C₁-C₄ alkyl, —C(═O)—C₁-C₄ alkyl, or 4- to 6-membered heterocycloalkyl, wherein the alkyl is unsubstituted or substituted by 1, 2, or 3 groups independently selected from —F, —CN, —OH, —CO₂H, —C(═NH)NH₂, and 5-membered monocyclic heteroaryl which is unsubstituted or substituted by 1 —CONH₂ group.

In some embodiments, R¹⁷ is 4- to 8-membered heterocycloalkyl, where the 4- to 8-membered heterocycloalkyl is unsubstituted or substituted by 1, 2, or 3 groups independently selected from: halogen, —OH, —OMe, —N(R⁸)₂, —NHSO₂R¹⁸, and —CH₂CN; and each R¹⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl.

In some embodiments, R¹⁷ is 4- to 6-membered heterocycloalkyl, where the 4- to 6-membered heterocycloalkyl is unsubstituted or substituted by 1, 2, or 3 groups independently selected from: —F, —OH, —OCH₃, and —NH₂.

In some embodiments, the compound is a compound of Formula (IX):

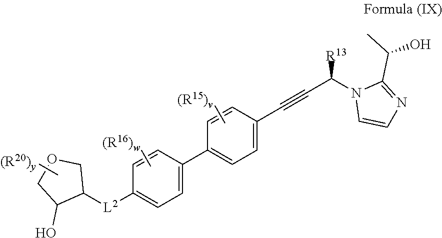

or a pharmaceutically acceptable salt, solvate, or stereoisomer thereof, wherein:
R¹³ is —(C₁-C₄ alkylene)-OH, —(C₁-C₄ alkylene)-NH₂, —(C₃-C₆ cycloalkylene)-OH, or —(C₃-C₆ cycloalkylene)-NH₂; each R¹⁵ and R¹⁶ is independently —F, —Cl, or C₁-C₄ alkyl; L² is —X³— or —X⁴—(C₁-C₆ alkylene)-; and X³ and X⁴ are each independently selected from: —N(R¹⁹)—, —S—, —S(═O)—, —S(═O)₂—, and —S(═O)(═NR¹⁹). R¹⁹ is hydrogen or C₁-C₆ alkyl; each R²⁰ is independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R¹⁸ is independently hydrogen or C₁-C₄ alkyl v is 0, 1, or 2; w is 0, 1, or 2; and y is 0, 1, or 2.

In some embodiments, the compound is a compound of Formula (X):

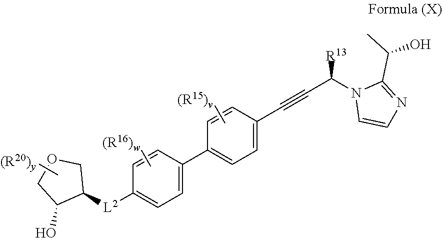

or a pharmaceutically acceptable salt, solvate, or stereoisomer thereof.

In some embodiments, R¹³ is —(C₁-C₄ alkylene)-OH. In some embodiments, R¹³ is —CH₂OH. In some embodiments, w and v are each 0.

In some embodiments, L² is a bond, —(C₁-C₆ alkylene)-, —O—, or —O—(C₁-C₆ alkylene)-; and
(i) R¹³ is —(C₃-C₆ cycloalkylene)-OH or —(C₃-C₆ cycloalkylene)-NH₂;
(ii) R¹⁴ is C₁-C₄ alkyl,
(iii) R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂; or
(iv) L² is —O—(C₁-C₆ alkylene)- and R¹⁷ is an unsubstituted or substituted 5-membered heterocycloalkyl

In some embodiments, R¹³ is —(C₃-C₆ cycloalkylene)-OH or —(C₃-C₆ cycloalkylene)-NH₂. In some embodiments, R¹³ is -(cyclopropylene)-OH or -(cyclopropylene)-NH₂. In some embodiments, R¹³ is

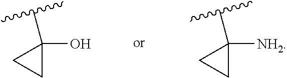 [SMILES: *C1(O)CC1 ; "or" ; *C1(N)CC1]

In some embodiments, R¹⁴ is C₁-C₄ alkyl. In some embodiments, R¹⁴ is methyl.

In some embodiments, R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂.

In some embodiments, R¹⁷ is an unsubstituted or substituted hexahydrofuro[3,2-b]furan, an unsubstituted or substituted tetrahydrothiophene-1-oxide, an unsubstituted or substituted 3-oxabicyclo[3.1.0]hexane, an unsubstituted or substituted tetrahydropyran, a disubstituted or trisubstituted cyclopropyl, or an oxetane substituted by at least one —N(R¹⁸).

In some embodiments, R¹⁷ is

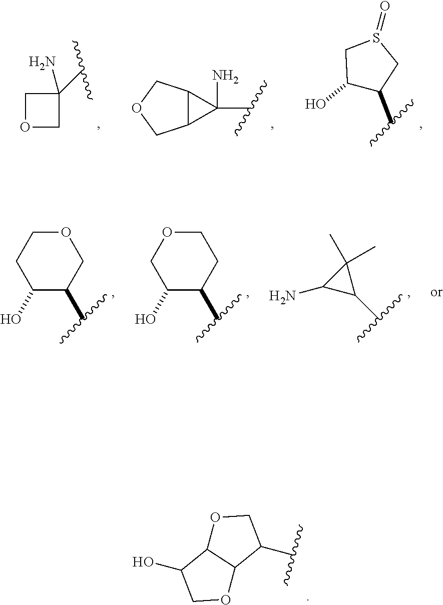

In some embodiments, L² is a bond, —CH₂—, —O—, or —O—CH₂—.

In some embodiments, -L²-R¹⁷ is

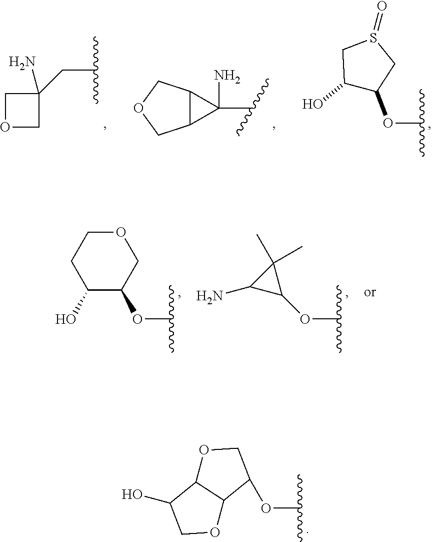

In some embodiments, L² is —O—(C₁-C₆ alkylene)-; and R¹⁷ is an unsubstituted or substituted 5-membered heterocycloalkyl. In some embodiments, R¹⁷ is an unsubstituted or substituted tetrahydrofuranyl.

In some embodiments, R¹⁷ is

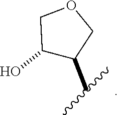 [SMILES: *[C@@H]1COC[C@H]1O]

In some embodiments, L² is —O—CH₂—. In some embodiments, -L²-R¹⁷ is

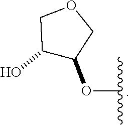 [SMILES: *O[C@@H]1COC[C@H]1O]

In another aspect, the present disclosure provides a pharmaceutical composition comprising a compound of Formula (I), (II), (III), (IV), (V), (VI), (VII), (VIII), (IX), (X), (XI), or (XII), or a pharmaceutically acceptable salt, solvate, or stereoisomer thereof, and a pharmaceutically acceptable excipient.

In another aspect, the present disclosure provides a method of treating or preventing a gram-negative bacterial infection in a patient in need thereof comprising administering to the patient a compound of Formula (I), (II), (III), (IV), (V), (VI), (VII), (VIII), (IX), (X), (XI), or (XII), or a pharmaceutically acceptable salt, solvate, or stereoisomer thereof, or a pharmaceutical composition comprising a compound of Formula (I), (II), (III), (IV), (V), (VI), (VII), (VIII), (IX), (X), (XI), or (XII), pharmaceutically acceptable salt, solvate, or stereoisomer thereof, and a pharmaceutically acceptable excipient.

In some embodiments, the gram-negative bacterial infection is associated with Pseudomonas aeruginosa. In some embodiments, the gram-negative bacterial infection is a respiratory infection. In some embodiments, the respiratory infection is pneumonia. In some embodiments, the pneumonia is community-acquired pneumonia (CAP), health care-associated pneumonia (HCAP), hospital-acquired pneumonia (HAP), ventilator-associate pneumonia (VAP), or a combination thereof.

In another aspect, the present disclosure provides a method of treating or preventing a P. aeruginosa infection in a patient in need thereof comprising administering to the patient a compound of Formula (I), (II), (III), (IV), (V), (VI), (VII), (VIII), (IX), (X), (XI), or (XII), or a pharmaceutically acceptable salt, solvate, or stereoisomer thereof, or a pharmaceutical composition comprising a compound of Formula (I), (II), (III), (IV), (V), (VI), (VII), (VIII), (IX), (X), (XI), or (XII), pharmaceutically acceptable salt, solvate, or stereoisomer thereof, and a pharmaceutically acceptable excipient.

In some embodiments, the patient has been identified as having a lung disease. In some embodiments, the lung disease is a structural lung disease. In some embodiments, the lung disease is cystic fibrosis, bronchiectasis, emphysema, chronic obstructive pulmonary disease (COPD), chronic destroyed lung disease, or a combination thereof. In some embodiments, the administration is to treat an existing infection. In some embodiments, the administration is provided as prophylaxis.

In some embodiments, the compound of Formula (I), (II), (III), (IV), (V), (VI), (VII), (VIII), (IX), (X), (XI), or (XII), or a pharmaceutically acceptable salt, solvate, or stereoisomer thereof, is administered in a solution by inhalation, intravenous injection, or intraperitoneal injection.

In some embodiments, the compound or salt is used as therapeutically active substance. In some embodiments, the compound or salt is used in treating or preventing a gram-negative bacterial infection. In some embodiments, the gram-negative bacterial infection is associated with Pseudomonas aeruginosa. In some embodiments, wherein the gram-negative bacterial infection is a respiratory infection. In some embodiments, the respiratory infection is pneumonia. In some embodiments, the pneumonia is community-acquired pneumonia (CAP), health care-associated pneumonia (HCAP), hospital-acquired pneumonia (HAP), ventilator-associate pneumonia (VAP), or a combination thereof. In some embodiments, the compound or salt is used in treating or preventing a P. aeruginosa infection. In some embodiments, the patient has been identified as having a lung disease. In some embodiments, the lung disease is a structural lung disease. In some embodiments, the lung disease is cystic fibrosis, bronchiectasis, emphysema, chronic obstructive pulmonary disease (COPD), chronic destroyed lung disease, or a combination thereof.

In another aspect, the present disclosure provides the use of a compound of Formula (I), (II), (III), (IV), (V), (VI), (VII), (VIII), (IX), (X), (XI), or (XII), or a pharmaceutically acceptable salt, solvate, or stereoisomer thereof, for the preparation of a medicament for treating or preventing a gram-negative bacterial infection.

In another aspect, the present disclosure provides the use of a compound of Formula (I), (II), (III), (IV), (V), (VI), (VII), (VIII), (IX), (X), (XI), or (XII), or a pharmaceutically acceptable salt, solvate, or stereoisomer thereof, for treating or preventing a gram-negative bacterial infection.

### INCORPORATION BY REFERENCE

All publications, patents, and patent applications mentioned in this specification are herein incorporated by reference for the specific purposes identified herein.

### DETAILED DESCRIPTION OF THE INVENTION

### LpxC, Lipid A and Gram-Negative Bacteria

Metalloproteins influence a vast diversity of biological systems, biological processes, and diseases. For example, UDP-{3-O—[(R)-3-hydroxymyristoyl]}-N-acetylglucosamine deacetylase (LpxC) is an essential enzyme involved in the first committed step in lipid A biosynthesis for gram-negative bacteria. Lipid A is an essential component of the outer membrane of gram-negative bacteria. LpxC is a zinc(II)-dependent metalloenzyme, with two histidines and an aspartic acid residue bound to the zinc(II) ion. Structures of LpxC show the zinc(II) ion is bound to two water molecules, both of which have been implicated in the mechanism of the enzyme. LpxC is highly conserved across strains of gram-negative bacteria, making LpxC an attractive target to treat gram-negative infections. To the contrary, LpxC is not a component of Gram-positive bacteria, such as Staphylococcus aureus.

In recent years, there has been an increase in resistant and multi-drug resistant strains of bacteria. Thus, there is a need for new antibiotics, especially with new mechanisms of action. There remains a need for metalloprotein modulators of LpxC useful in the field of therapeutics, diagnostics, and research.

Some embodiments provide a method of inhibiting UDP-{3-O—[(R)-3-hydroxymyristoyl]}-N-acetylglucosamine deacetylase enzyme comprising contacting the enzyme with a compound of Formula (I).

In some embodiments provided herein is a pharmaceutical composition comprising a compound of Formula (I) or a pharmaceutically acceptable salt thereof, and a pharmaceutically acceptable excipient.

### Methods of Use

Disclosed herein are methods of treating disease wherein the inhibition of bacterial growth is indicated. Such disease includes gram-negative bacterial infection. In some embodiments, the gram-negative bacterial infection is associated with Pseudomonas aeruginosa. In some embodiments, the method of treating a gram-negative bacterial infection in a patient in need thereof comprises administering to the patient a compound of Formula (I), a pharmaceutical composition comprising a compound of Formula (I), or a pharmaceutically acceptable salt thereof, and a pharmaceutically acceptable excipient. In some embodiments, the method of treating a Pseudomonas aeruginosa infection in a patient in need thereof comprises administering to the patient the compound of Formula (I), a pharmaceutical composition comprising a compound of Formula (I), or a pharmaceutically acceptable salt thereof, and a pharmaceutically acceptable excipient.

In some embodiments, the gram-negative bacterial infection is associated with Pseudomonas aeruginosa. In some embodiments, the gram-negative bacterial infection is a respiratory infection. In some embodiments, the gram-negative bacterial infection is pneumonia. In some embodiments, the gram-negative bacterial infection is community-acquired pneumonia (CAP), health care-associated pneumonia (HCAP), hospital-acquired pneumonia (HAP), ventilator-associate pneumonia (VAP), or a combination thereof. In some embodiments, the gram-negative bacterial infection is community-acquired pneumonia (CAP). In some embodiments, the gram-negative bacterial infection is health care-associated pneumonia (HCAP). In some embodiments, the gram-negative bacterial infection is hospital-acquired pneumonia (HAP). In some embodiments, the gram-negative bacterial infection is ventilator-associate pneumonia (VAP).

In some embodiments, the patient has been identified as having a lung disease. In some embodiments, the lung disease is a structural lung disease. In some embodiments, the lung disease is cystic fibrosis, bronchiectasis, emphysema, chronic obstructive pulmonary disease (COPD), chronic destroyed lung disease, or a combination thereof. In some embodiments, the patient has cystic fibrosis. In some embodiments, the patient has bronchiectasis. In some embodiments, the patient has emphysema. In some embodiments, the patient has chronic obstructive pulmonary disease (COPD). In some embodiments, the patient has chronic destroyed lung disease.

In some embodiments the administration is to treat an existing infection.

In some embodiments the administration is provided as prophylaxis.

In some embodiments, the LpxC inhibitory compound as described herein is used for treating or preventing conditions caused by the bacterial production of endotoxin and, in particular, by gram-negative bacteria and bacteria that use LpxC in the biosynthesis of lipopolysaccharide (LPS) or endotoxin. In some embodiments, the method of treating or preventing a condition caused by endotoxin or LPS in a patient in need thereof comprises administering to the patient a pharmaceutical composition comprising a compound of Formula (I), or a pharmaceutically acceptable salt thereof, and a pharmaceutically acceptable excipient. In another embodiment, the heterocyclic LpxC inhibitory compounds as described herein are useful in the treatment of conditions that are caused or exacerbated by the bacterial production of lipid A and LPS or endotoxin, such as chronic obstructive pulmonary disease (COPD) an d acute exacerbations of chronic bronchitis (AECB). In some embodiments, the method of treating or preventing a condition caused by endotoxin or LPS in a patient in need thereof comprises administering to the patient a pharmaceutical composition comprising a compound of Formula (I), or a pharmaceutically acceptable salt thereof, and a pharmaceutically acceptable excipient, wherein the condition caused by endotoxin or LPS is selected from chronic obstructive pulmonary disease (COPD) and acute exacerbations of chronic bronchitis (AECB).

In other embodiments, the compounds of the disclosure can be used for the treatment of a serious or chronic respiratory tract infection including serious lung and nosocomial infections such as those caused by Enterobacter aerogenes, Enterobacter cloacae, Escherichia coli, Klebsiella pneumoniae, Klebsiella oxytoca, Kuyvera ascorbata, Kuyvera cryocrescense, Shigella sonnei, Proteus mirabilis, Serratia marcescens, Stenotrophomonas maltophilia, Pseudomonas aeruginosa, Burkholderia cepacia, Acinetobacter baumannii, Alcaligenes xylosoxidans, Flavobacterium meningosepticum, and Citrobacter freundi, Haemophilus influenzae, Kluyvera species, Legionella species, Moraxella catarrhalis, Enterobacter species, Acinetobacter species, Klebsiella species, Burkholderia species and Proteus species, and infections caused by other bacterial species such as Neisseria species, Shigella species, Salmonella species, Helicobacter pylori, Vibrionaceae and Bordetella species as well as the infections caused by a Brucella species, Francisella tularensis and/or Yersinia pestis. In some embodiments, the infection is associated with a Pseudomonas species. In some embodiments, the infection is associated with Pseudomonas aeruginosa. In some embodiments, the compounds of the disclosure do not inhibit the growth of Gram-positive bacteria, such as Staphylococcus aureus.

In some embodiments, the LpxC inhibitory compound as described herein is used in a method of preventing growth of a Pseudomonas species. In some embodiments, the Pseudomonas species is Pseudomonas aeruginosa.

In some instances, antibiotics have suboptimal concentrations in the lung leading to therapeutic failures for lung infections. In some embodiments, the heterocyclic LpxC inhibitory compound of Formula (I) have optimal concentrations in the lung for treating or preventing a gram-negative bacterial infection in the lung. In some embodiments, the compounds are present in the lung in a therapeutically effective amount after administration.

In some embodiments, disclosed herein is a compound described herein, or a pharmaceutically acceptable salt thereof, for use as therapeutically active substance.

In some embodiments, disclosed herein is a compound described herein, or a pharmaceutically acceptable salt thereof, for use in treating or preventing a gram-negative bacterial infection. In some embodiments, the gram-negative bacterial infection is associated with Pseudomonas aeruginosa. In some embodiments, the gram-negative bacterial infection is a respiratory infection. In some embodiments, the respiratory infection is pneumonia.

In some embodiments, disclosed herein is the use of a compound described herein, or a pharmaceutically acceptable salt thereof, for the preparation of a medicament for treating or preventing a gram-negative bacterial infection. In some embodiments, the gram-negative bacterial infection is associated with Pseudomonas aeruginosa. In some embodiments, the gram-negative bacterial infection is a respiratory infection. In some embodiments, the respiratory infection is pneumonia.

### LpxC Inhibitory Compounds

Provided herein, in some embodiments, are heterocyclic LpxC inhibitory compounds and pharmaceutical compositions comprising said compounds. The subject compounds and compositions are useful for inhibiting UDP-{3-O—[(R)-3-hydroxymyristoyl]}-N-acetylglucosamine deacetylase (LpxC) and for the treatment of bacterial infection.

In some embodiments, compounds of Formula (I), including pharmaceutically acceptable salts, prodrugs, active metabolites, and pharmaceutically acceptable solvates thereof, are UDP-{3-O—[(R)-3-hydroxymyristoyl]}-N-acetylglucosamine deacetylase (LpxC) modulators. In some embodiments, the compounds of Formula (I), including pharmaceutically acceptable salts, prodrugs, active metabolites, and pharmaceutically acceptable solvates thereof, are UDP-{3-O—[(R)-3-hydroxymyristoyl]}-N-acetylglucosamine deacetylase (LpxC) antagonists. In some embodiments, the compounds of Formula (I), including pharmaceutically acceptable salts, prodrugs, active metabolites, and pharmaceutically acceptable solvates thereof, are UDP-{3-O—[(R)-3-hydroxymyristoyl]}-N-acetylglucosamine deacetylase (LpxC) inhibitors.

One aspect of the disclosure provides a compound having the structure of Formula (I):

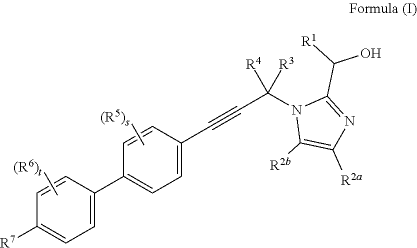

or a pharmaceutically acceptable salt, solvate, or stereoisomer thereof, wherein:
R¹ is C₁-C₄ alkyl;
R^{2a} and R^{2b} are each independently hydrogen, halogen, or C₁-C₄ alkyl;
R³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂;
R⁴ is hydrogen or C₁-C₄ alkyl;
each R⁵ and R⁶ is independently halogen, or C₁-C₄ alkyl;
L¹ is a bond, —(C₁-C₆ alkylene)-, —X¹—, or —X²—(C₁-C₆ alkylene)-, wherein:
   X¹ and X² are each selected from: —O—, —N(R⁹)—, —S—, —S(═O)—, —S(═O)₂—, and —S(═O)(═NR⁹)—, wherein:
      R⁹ is hydrogen or C₁-C₆ alkyl;
R⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R¹⁰ groups, wherein:
each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo;
further wherein:
each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃;
or two R⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo;
s is 0, 1 or 2; and
t is 0, 1 or 2.
   In some embodiments, for the compound of Formula (I),
(i) s is 1 or 2, and at least one R⁵ is halogen;
(ii) t is 1 or 2, and at least one R⁶ is halogen; and
(iii) the alkyl, heteroalkyl, cycloalkyl, or heterocycloalkyl of R⁷ is substituted by 1, 2, or 3 R¹⁰ groups, and at least one R¹⁰ is halogen.

In some embodiments, for the compound of Formula (I), s is 1 or 2, and at least one R⁵ is halogen.

In some embodiments, for the compound of Formula (I), t is 1 or 2, and at least one R⁶ is halogen.

In some embodiments, for the compound of Formula (I), the alkyl, heteroalkyl, cycloalkyl, or heterocycloalkyl of R⁷ is substituted by 1, 2, or 3 R¹⁰ groups, and at least one R¹⁰ is halogen.

In some embodiments, for the compound of Formula (I), R¹ is C₁-C₄ alkyl; R^{2a} and R^{2b} are each independently hydrogen, halogen, or C₁-C₄ alkyl; R³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂; R⁴ is hydrogen or C₁-C₄ alkyl; each R⁵ and R⁶ is independently halogen or C₁-C₄ alkyl; L¹ is a bond, —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and S(═O)(═NR⁹)—, wherein: R⁹ is hydrogen or C₁-C₆ alkyl; R⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R¹⁰ groups, wherein: each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, or 4- to 6-membered heterocycloalkyl, wherein the alkyl or heterocycloalkyl is unsubstituted or substituted by 1, 2, or 3 groups independently selected from —F, —CN, —OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo; s is 0, 1 or 2; t is 0, 1 or 2; and wherein at least one of the following: (i) s is 1 or 2, and at least one R⁵ is halogen; (ii) t is 1 or 2, and at least one R⁶ is halogen; and (iii) the alkyl, heteroalkyl, cycloalkyl, or heterocycloalkyl of R⁷ is substituted by 1, 2, or 3 R¹⁰ groups, and at least one R¹⁰ is halogen.

In some embodiments, the compound of Formula (I) is a compound of Formula (Ia) or Formula (Ib):

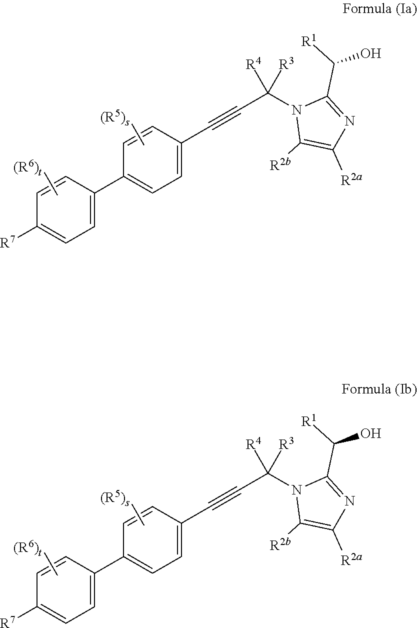

or a pharmaceutically acceptable salt, solvate, or stereoisomer thereof.

In some embodiments of a compound of Formula (I), (Ia), or (Ib), R¹ is —CH₃; R^{2a} and R^{2b} are each hydrogen; R³ is hydrogen, —(C₁-C₄ alkylene)-OH, or —(C₁-C₄ alkylene)-NH₂; R⁴ is hydrogen; each R⁵ and R⁶ is —F; L¹ is a bond, —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and S(═O)(═NR⁹)—, wherein: R⁹ is hydrogen or C₁-C₆ alkyl; R⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R¹⁰ groups, wherein: each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; wherein at least one of the following: (i) s is 1 or 2; (ii) t is 1 or 2; and (iii) the alkyl, heteroalkyl, cycloalkyl, or heterocycloalkyl of R⁷ is substituted by 1, 2, or 3 R¹⁰ groups, and at least one R¹⁰ is fluorine.

In some embodiments of a compound of Formula (I), (Ia), or (Ib), R¹ is —CH₃; R^{2a} and R^{2b} are each hydrogen; R³ is hydrogen, —(C₁-C₄ alkylene)-OH, or —(C₁-C₄ alkylene)-NH₂; R⁴ is hydrogen; each R⁵ and R⁶ is —F; L¹ is a bond, —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and S(═O)(═NR⁹)—, wherein: R⁹ is hydrogen or C₁-C₆ alkyl; R⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R¹⁰ groups, wherein: each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; wherein at least one of the following: (i) s is 1 or 2; (ii) t is 1 or 2; and (iii) the alkyl, heteroalkyl, cycloalkyl, or heterocycloalkyl of R⁷ is substituted by 1, 2, or 3 R¹⁰ groups, and at least one R¹⁰ is fluorine.

In some embodiments of a compound of Formula (I), (Ia), or (Ib), R¹ is —CH₃; R^{2a} and R^{2b} are each hydrogen; R³ is hydrogen or —(C₁-C₄ alkylene)-OH; R⁴ is hydrogen; each R⁵ and R⁶ is —F; L¹ is a bond, —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and S(═O)(═NR⁹)—, wherein: R⁹ is hydrogen or C₁-C₆ alkyl; R⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R¹⁰ groups, wherein: each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; wherein at least one of the following: (i) s is 1 or 2; (ii) t is 1 or 2; and (iii) the alkyl, heteroalkyl, cycloalkyl, or heterocycloalkyl of R⁷ is substituted by 1, 2, or 3 R¹⁰ groups, and at least one R¹⁰ is fluorine.

In some embodiments of a compound of Formula (I), (Ia), or (Ib), R¹ is —CH₃; R^{2a} and R^{2b} are each hydrogen; R³ is hydrogen or —(C₁-C₄ alkylene)-OH; R⁴ is hydrogen; each R⁵ and R⁶ is —F; L¹ is a bond, —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and S(═O)(═NR⁹)—, wherein: R⁹ is hydrogen or C₁-C₆ alkyl; R⁷ is 4- to 8-membered heterocycloalkyl, where the 4- to 8-membered heterocycloalkyl is unsubstituted or substituted by 1, 2, or 3 R¹⁰ groups, wherein: each R¹⁰ is independently selected from: halogen, —OR⁸, —N(R⁸)₂, —NHSO₂R⁸, —CH₂CN, C₁-C₄ alkyl, —C(═O)—C₁-C₄ hydroxyalkyl, C₁-C₄ hydroxyalkyl, and C₁-C₄ methoxyalkyl; and each R⁸ is independently hydrogen, C₁-C₄ alkyl, —C(═O)—C₁-C₄ alkyl, or 4- to 6-membered heterocycloalkyl, wherein the alkyl is unsubstituted or substituted by 1, 2, or 3 groups independently selected from —F, —CN, —OH, —CO₂H, —C(═NH)NH₂, and 5-membered monocyclic heteroaryl which is unsubstituted or substituted by 1 —CONH₂ group; wherein at least one of the following: (i) s is 1 or 2; (ii) t is 1 or 2; and (iii) the alkyl, heteroalkyl, cycloalkyl, or heterocycloalkyl of R⁷ is substituted by 1, 2, or 3 R¹⁰ groups, and at least one R¹⁰ is fluorine.

In some embodiments of a compound of Formula (I), (Ia), or (Ib), R¹ is —CH₃; R^{2a} and R^{2b} are each hydrogen; R³ is hydrogen or —CH₂OH; R⁴ is hydrogen; each R⁵ and R⁶ is —F; L¹ is a bond, —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and S(═O)(═NR⁹)—, wherein: R⁹ is hydrogen or C₁-C₆ alkyl; R⁷ is 4- to 8-membered heterocycloalkyl, where the 4- to 8-membered heterocycloalkyl is unsubstituted or substituted by 1, 2, or 3 R¹⁰ groups, wherein: each R¹⁰ is independently selected from: halogen, —OH, —OMe, —N(R⁸)₂, —NHSO₂R⁸, and —CH₂CN; and each R⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl; wherein at least one of the following: (i) s is 1 or 2; (ii) t is 1 or 2; and (iii) the alkyl, heteroalkyl, cycloalkyl, or heterocycloalkyl of R⁷ is substituted by 1, 2, or 3 R¹⁰ groups, and at least one R¹⁰ is fluorine.

In some embodiments of a compound of Formula (I), (Ia), or (Ib), R¹ is —CH₃; R^{2a} and R^{2b} are each hydrogen; R³ is hydrogen or —CH₂OH; R⁴ is hydrogen; each R⁵ and R⁶ is —F; L¹ is a bond, —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and S(═O)(═NR⁹)—, wherein: R⁹ is hydrogen or C₁-C₆ alkyl; R⁷ is 4- to 6-membered heterocycloalkyl, where the 4- to 6-membered heterocycloalkyl is unsubstituted or substituted by 1, 2, or 3 R¹⁰ groups, wherein: each R¹⁰ is independently selected from: —F, —OH, —OCH₃, and —NH₂; s is 0, 1, or 2; t is 0, 1, or 2; wherein at least one of the following: (i) s is 1 or 2; (ii) t is 1 or 2; and (iii) the alkyl, heteroalkyl, cycloalkyl, or heterocycloalkyl of R⁷ is substituted by 1, 2, or 3 R¹⁰ groups, and at least one R¹⁰ is fluorine.

In some embodiments, the compound is a compound of Formula (I), or a pharmaceutically acceptable salt, solvate, or stereoisomer thereof. In some embodiments, the compound is a compound of Formula (Ia), or a pharmaceutically acceptable salt, solvate, or stereoisomer thereof. In some embodiments, the compound is a compound of Formula (Ib), or a pharmaceutically acceptable salt, solvate, or stereoisomer thereof.

For any and all of the embodiments, substituents are selected from among a subset of the listed alternatives. For example, in some embodiments of a compound of Formula (I), (Ia), or (Ib), R¹ is unsubstituted C₁-C₄ alkyl. In some embodiments, R¹ is C₁-C₂ alkyl. In some embodiments, R¹ is —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃. In some embodiments, R¹ is —CH₃ or —CH₂CH₃. In some embodiments, R¹ is —CH₃.

In some embodiments of a compound of Formula (I), (Ia), or (Ib), R^{2a} and R^{2b} are each independently R^{2a} and R^{2b} are each independently hydrogen, halogen, or unsubstituted C₁-C₄ alkyl. In some embodiments, R^{2a} and R^{2b} are each independently hydrogen, —F, —Cl, —Br, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, R^{2a} and R^{2b} are each independently hydrogen, —F, —Cl, —CH₃, —CH₂CH₃, or —CH(CH₃)₂.

In some embodiments of a compound of Formula (I), (Ia), or (Ib), R^{2a} is hydrogen. In some embodiments, R^{2b} is hydrogen. In some embodiments, R^{2a} and R^{2b} are each hydrogen.

In some embodiments of a compound of Formula (I), (Ia), or (Ib), R¹ is —CH₃; R^{2a} is hydrogen; and R^{2b} is hydrogen.

In some embodiments of a compound of Formula (I), (Ia), or (Ib), R⁴ is hydrogen or unsubstituted C₁-C₄ alkyl. In some embodiments, R⁴ is hydrogen or C₁-C₂ alkyl. In some embodiments, R⁴ is hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃. In some embodiments, R⁴ is hydrogen, —CH₃ or —CH₂CH₃. In some embodiments, R⁴ is hydrogen or —CH₃. In some embodiments, R⁴ is hydrogen. In some embodiments, R⁴ is —CH₃.

In some embodiments, the compound of Formula (I) is a compound of Formula (II):

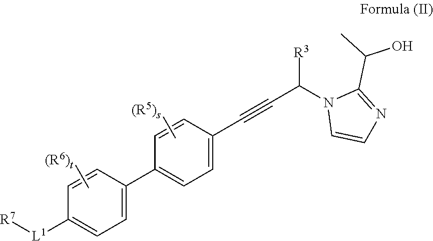

or a pharmaceutically acceptable salt, solvate, or stereoisomer thereof.

In some embodiments of a compound of Formula (II), R³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂; each R⁵ is halogen or C₁-C₄ alkyl; each R⁶ is halogen or C₁-C₄ alkyl; L¹ is a bond, —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and —S(═O)(═NR⁹)—, wherein: R⁹ is hydrogen or C₁-C₆ alkyl; R⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R¹⁰ groups, wherein: each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo; s is 0, 1, or 2; t is 0, 1, or 2; and wherein at least one of the following: (i) s is 1 or 2, and at least one R⁵ is halogen; (ii) t is 1 or 2, and at least one R⁶ is halogen; and (iii) the alkyl, heteroalkyl, cycloalkyl, or heterocycloalkyl of R⁷ is substituted by 1, 2, or 3 R¹⁰ groups, and at least one R¹⁰ is halogen.

In some embodiments, R³ is hydrogen, —(C₁-C₄ alkylene)-OH, or —(C₁-C₄ alkylene)-NH₂; R⁵ is —F; R⁶ is —F; L¹ is a bond, —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and —S(═O)(═NR⁹)—, wherein: R⁹ is hydrogen or C₁-C₆ alkyl; R⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R¹⁰ groups, wherein: each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; s is 0, 1, or 2; t is 0, 1, or 2; and wherein at least one of the following: (i) s is 1 or 2, and at least one R⁵ is halogen; (ii) t is 1 or 2, and at least one R⁶ is halogen; and (iii) the alkyl, heteroalkyl, cycloalkyl, or heterocycloalkyl of R⁷ is substituted by 1, 2, or 3 R¹⁰ groups, and at least one R¹⁰ is halogen.

In some embodiments of a compound of Formula (II), each R⁵ is independently halogen or C₁-C₄ alkyl; and each R⁶ is independently halogen or C₁-C₄ alkyl. In some embodiments, each R⁵ is independently halogen; and each R⁶ is independently halogen. In some embodiments, each R⁵ is independently —F; and each R⁶ is independently —F. In some embodiments, each R⁵ is independently —Cl; and each R⁶ is independently —Cl.

In some embodiments, the compound of Formula (I) or (II) is a compound of Formula (IIa) or Formula (IIb):

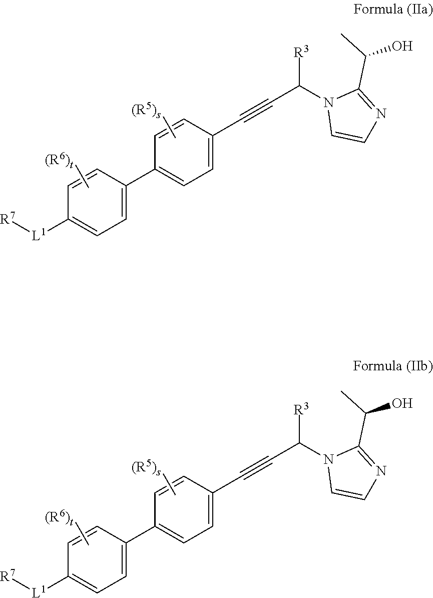

or a pharmaceutically acceptable salt, solvate, or stereoisomer thereof.

In some embodiments of a compound of Formula (II), (IIa), or (IIb), R³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂; each R⁵ is halogen or C₁-C₄ alkyl; each R⁶ is halogen or C₁-C₄ alkyl; L¹ is a bond, —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and —S(═O)(═NR⁹)—, wherein: R⁹ is hydrogen or C₁-C₆ alkyl; R⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R¹⁰ groups, wherein: each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo; s is 0, 1, or 2; t is 0, 1, or 2; and wherein at least one of the following: (i) s is 1 or 2, and at least one R⁵ is halogen; (ii) t is 1 or 2, and at least one R⁶ is halogen; and (iii) the alkyl, heteroalkyl, cycloalkyl, or heterocycloalkyl of R⁷ is substituted by 1, 2, or 3 R¹⁰ groups, and at least one R¹⁰ is halogen.

In some embodiments of a compound of Formula (II), (IIa), or (IIb), R³ is hydrogen, —(C₁-C₄ alkylene)-OH, or —(C₁-C₄ alkylene)-NH₂; R⁵ is —F; R⁶ is —F; L¹ is a bond, —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and —S(═O)(═NR⁹)—, wherein: R⁹ is hydrogen or C₁-C₆ alkyl; R⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R¹⁰ groups, wherein: each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; s is 0, 1, or 2; t is 0, 1, or 2; and wherein at least one of the following: (i) s is 1 or 2, and at least one R⁵ is halogen; (ii) t is 1 or 2, and at least one R⁶ is halogen; and (iii) the alkyl, heteroalkyl, cycloalkyl, or heterocycloalkyl of R⁷ is substituted by 1, 2, or 3 R¹⁰ groups, and at least one R¹⁰ is halogen.

In some embodiments of a compound of Formula (II), (IIa), or (IIb), each R⁵ is independently halogen or C₁-C₄ alkyl; and each R⁶ is independently halogen or C₁-C₄ alkyl. In some embodiments, each R⁵ is independently halogen; and each R⁶ is independently halogen. In some embodiments, each R⁵ is independently —F; and each R⁶ is independently —F.

In some embodiments of a compound of Formula (II), (IIa), or (IIb), each R⁵ is —F. In some embodiments of a compound of Formula (II), (IIa), or (IIb), each R⁵ is —Cl.

In some embodiments of a compound of Formula (II), (IIa), or (IIb), each R⁶ is —F. In some embodiments of a compound of Formula (II), (IIa), or (IIb), each R⁶ is —Cl.

In some embodiments of a compound of Formula (II), (IIa), or (IIb), s is 0; and each R⁶ is —F. In some embodiments, each R⁵ is —F; and t is 0. In some embodiments, R⁵ is —F; and R⁶ is —F.

In some embodiments of a compound of Formula (II), (IIa), or (IIb), s is 0; and each R⁶ is —Cl. In some embodiments, each R⁵ is —Cl; and t is 0. In some embodiments, R⁵ is —Cl; and R⁶ is —Cl.

In some embodiments, the compound is a compound of Formula (II), or a pharmaceutically acceptable salt, solvate, or stereoisomer thereof. In some embodiments, the compound is a compound of Formula (IIa), or a pharmaceutically acceptable salt, solvate, or stereoisomer thereof. In some embodiments, the compound is a compound of Formula (IIb), or a pharmaceutically acceptable salt, solvate, or stereoisomer thereof.

In some embodiments, the compound of Formula (I) or (II) is a compound of Formula (IIIa), Formula (IIIb), Formula (IIIc), or Formula (IIId):

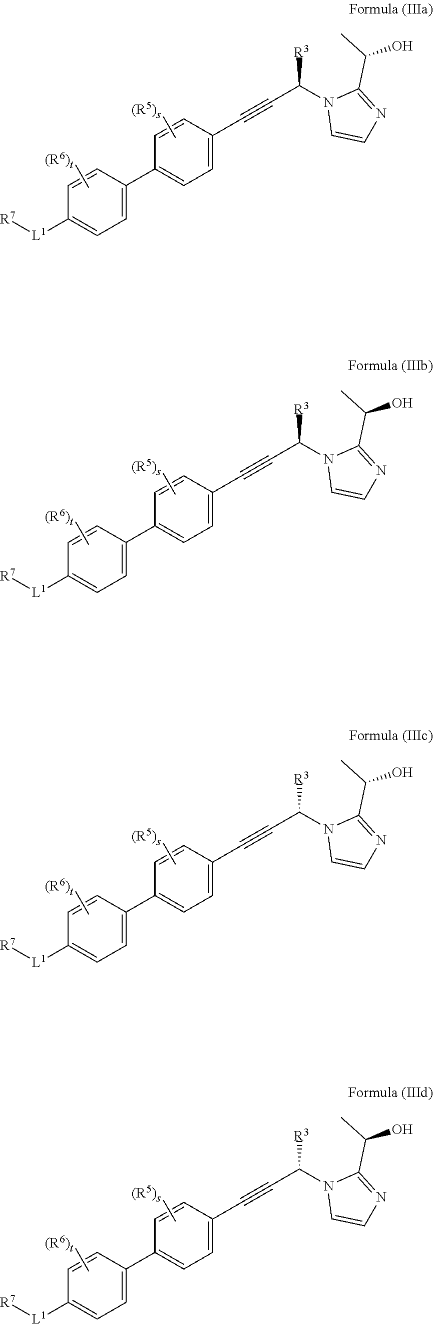

or a pharmaceutically acceptable salt, solvate, or stereoisomer thereof.

In some embodiments of a compound of Formula (IIIa), (IIIb), (IIIc) or (IIId), R³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂; each R⁵ is halogen or C₁-C₄ alkyl; each R⁶ is halogen or C₁-C₄ alkyl; L¹ is a bond, —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and —S(═O)(═NR⁹)—, wherein: R⁹ is hydrogen or C₁-C₆ alkyl; R⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R¹⁰ groups, wherein: each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo; s is 0, 1, or 2; t is 0, 1, or 2; and wherein at least one of the following: (i) s is 1 or 2, and at least one R⁵ is halogen; (ii) t is 1 or 2, and at least one R⁶ is halogen; and (iii) the alkyl, heteroalkyl, cycloalkyl, or heterocycloalkyl of R⁷ is substituted by 1, 2, or 3 R¹⁰ groups, and at least one R¹⁰ is halogen.

In some embodiments of a compound of Formula (IIIa), (IIIb), (IIIc) or (IIId), R³ is hydrogen, —(C₁-C₄ alkylene)-OH, or —(C₁-C₄ alkylene)-NH₂; R⁵ is —F; R⁶ is —F; L¹ is a bond, —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and —S(═O)(═NR⁹)—, wherein: R⁹ is hydrogen or C₁-C₆ alkyl; R⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R¹⁰ groups, wherein: each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; s is 0, 1, or 2; t is 0, 1, or 2; and wherein at least one of the following: (i) s is 1 or 2, and at least one R⁵ is halogen; (ii) t is 1 or 2, and at least one R⁶ is halogen; and (iii) the alkyl, heteroalkyl, cycloalkyl, or heterocycloalkyl of R⁷ is substituted by 1, 2, or 3 R¹⁰ groups, and at least one R¹⁰ is halogen.

In some embodiments of a compound of Formula (IIIa), (IIIb), (IIIc) or (IIId), each R⁵ is independently halogen or C₁-C₄ alkyl; and each R⁶ is independently halogen or C₁-C₄ alkyl. In some embodiments, each R⁵ is independently halogen; and each R⁶ is independently halogen. In some embodiments, each R⁵ is independently —F; and each R⁶ is independently —F. In some embodiments, each R⁵ is independently —Cl; and each R⁶ is independently —Cl.

In some embodiments of a compound of Formula (IIIa), (IIIb), (IIIc) or (IIId), each R⁵ is —F. In some embodiments of a compound of Formula (IIIa), (IIIb), (IIIc) or (IIId), each R⁵ is —Cl.

In some embodiments of a compound of Formula (IIIa), (IIIb), (IIIc) or (IIId), each R⁶ is —F. In some embodiments of a compound of Formula (IIIa), (IIIb), (IIIc) or (IIId), each R⁶ is —Cl.

In some embodiments of a compound of Formula (IIIa), (IIIb), (IIIc) or (IIId), s is 0; and each R⁶ is —F. In some embodiments, each R⁵ is —F; and t is 0. In some embodiments, R⁵ is —F; and R⁶ is —F.

In some embodiments of a compound of Formula (IIIa), (IIIb), (IIIc) or (IIId), s is 0; and each R⁶ is —Cl. In some embodiments, each R⁵ is —Cl; and t is 0. In some embodiments, R⁵ is —Cl; and R⁶ is —Cl.

In some embodiments, the compound is a compound of Formula (IIIa), or a pharmaceutically acceptable salt, solvate, or stereoisomer thereof. In some embodiments, the compound is a compound of Formula (IIIb), or a pharmaceutically acceptable salt, solvate, or stereoisomer thereof. In some embodiments, the compound is a compound of Formula (IIIc), or a pharmaceutically acceptable salt, solvate, or stereoisomer thereof. In some embodiments, the compound is a compound of Formula (IIId), or a pharmaceutically acceptable salt, solvate, or stereoisomer thereof.

In some embodiments of a compound of Formula (I), (Ia), (Ib), (II), (IIa), (IIb), (IIIa), (IIIb), (IIIc), or (IIId), L¹ is a bond, —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and —S(═O)(═NR⁹)—, wherein: R⁹ is hydrogen or C₁-C₆ alkyl. In some embodiments, L¹ is a bond, —(C₁-C₃ alkylene)-, —X¹—, or —X²—(C₁-C₆ alkylene)-, wherein: X¹ and X² are each selected from: —O—, —N(R⁹)—, —S—, —S(═O)—, —S(═O)₂—, and —S(═O)(═NR⁹)—. In some embodiments, L¹ is a bond, —(C₁-C₂ alkylene)-, —X¹—, or —X²—(C₁-C₆ alkylene)-, wherein: X¹ and X² are each selected from: —O—, —N(R⁹)—, —S—, —S(═O)—, —S(═O)₂—, and —S(═O)(═NR⁹)—. In some embodiments, L¹ is a bond, —CH₂—, —X¹—, or —X²—(C₁-C₆ alkylene)-, wherein: X¹ and X² are each selected from: —O—, —N(R⁹)—, —S—, —S(═O)—, —S(═O)₂—, and —S(═O)(═NR⁹)—. In some embodiments, each R⁹ is independently hydrogen or methyl. In some embodiments, R⁹ is hydrogen.

In some embodiments of a compound of Formula (I), (Ia), (Ib), (II), (IIa), (IIb), (IIIa), (IIIb), (IIIc), or (IIId), L¹ is —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and —S(═O)(═NR⁹)—. In some embodiments, L¹ is —CH₂—, —X¹—, or —X²—(C₁-C₆ alkylene)-, wherein: X¹ and X² are each selected from: —O—, —N(R⁹)—, —S—, —S(═O)—, —S(═O)₂—, and —S(═O)(═NR⁹)—. In some embodiments, L¹ is a bond, —X¹—, or —X²—(C₁-C₆ alkylene)-, wherein: X¹ and X² are each selected from: —O—, —N(R⁹)—, —S—, —S(═O)—, —S(═O)₂—, and —S(═O)(═NR⁹)—. In some embodiments, R⁹ is hydrogen.

In some embodiments, L¹ is a —X¹—, or —X²—(C₁-C₆ alkylene)-, wherein: X¹ and X² are each selected from: —O—, —N(R⁹)—, —S—, —S(═O)—, —S(═O)₂—, and —S(═O)(═NR⁹)—. In some embodiments, L¹ is a —X¹—, or —X²—(C₁-C₆ alkylene)-, wherein: X¹ and X² are each selected from: —O—, —N(R⁹)—, —S—, —S(═O)—, and —S(═O)₂—. In some embodiments, L¹ is a —X¹—, or —X²—(C₁-C₆ alkylene)-, wherein: X¹ and X² are each selected from: —O—, —N(R⁹)— and —S—. In some embodiments, L¹ is a —X¹—, or —X²—(C₁-C₆ alkylene)-, wherein: X¹ and X² are each selected from: —O— and —N(R⁹)—. In some embodiments, L¹ is a —X¹—, or —X²—(C₁-C₆ alkylene)-, wherein: X¹ and X² are each selected from: —O— and —S—. In some embodiments, L¹ is a —X¹—, or —X²—(C₁-C₆ alkylene)-, wherein: X¹ and X² are each selected from: —N(R⁹)— and —S—. In some embodiments, R⁹ is hydrogen.

In some embodiments of a compound of Formula (I), (Ia), (Ib), (II), (IIa), (IIb), (IIIa), (IIIb), (IIIc), or (IIId), L¹ is —X¹— or —X²—(C₁-C₄ alkylene)-, wherein: X¹ and X² are each selected from: —O—, —N(H)—, —S—, —S(═O)—, —S(═O)₂—, and —S(═O)(═NH)—. In some embodiments, L¹ is —X¹— or —X²—(C₁-C₄ alkylene)-, wherein: X¹ and X² are each selected from: —O—. In some embodiments, L¹ is —O—.

In some embodiments of a compound of Formula (I), (Ia), (Ib), (II), (IIa), (IIb), (IIIa), (IIIb), (IIIc), or (IIId), L¹ is —O— or —O—(C₁-C₆ alkylene)-. In some embodiments, L¹ is —N(R⁹)— or —N(R⁹)—(C₁-C₆ alkylene)-. In some embodiments, L¹ is —S— or —S—(C₁-C₆ alkylene)-. In some embodiments, L¹ is —S(═O)— or —S(═O)—(C₁-C₆ alkylene)-. In some embodiments, L¹ is —S(═O)₂— or —S(═O)₂—(C₁-C₆ alkylene)-. In some embodiments, L¹ is —S(═O)(═NR⁹)— or —S(═O)(═NR⁹)—(C₁-C₆ alkylene)-. In some embodiments, L¹ is —O—. In some embodiments, L¹ is —N(R⁹)—. In some embodiments, L¹ is —S—. In some embodiments, L¹ is —S(═O)—. In some embodiments, L¹ is —S(═O)₂—. In some embodiments, L¹ is —S(═O)(═NR⁹)—. In some embodiments, L¹ is —O—(C₁-C₆ alkylene)-. In some embodiments, L¹ is —N(R⁹)—(C₁-C₆ alkylene)-. In some embodiments, L¹ is —S—(C₁-C₆ alkylene)-. In some embodiments, L¹ is —S(═O)—(C₁-C₆ alkylene)-. In some embodiments, L¹ is —S(═O)₂—(C₁-C₆ alkylene)-. In some embodiments, L¹ is —S(═O)(═NR⁹)—(C₁-C₆ alkylene)-. In some embodiments, L¹ is —O— or —O—(CH₂)—. In some embodiments, L¹ is —N(H)— or —N(H)—(CH₂)—. In some embodiments, L¹ is —S— or —S—(CH₂)—. In some embodiments, L¹ is —S(═O)— or —S(═O)—(CH₂)—. In some embodiments, L¹ is —S(═O)₂— or —S(═O)₂—(CH₂)—. In some embodiments, L¹ is —S(═O)(═NH)— or —S(═O)(═NH)—(CH₂)—. In some embodiments, L¹ is —O—(CH₂)—. In some embodiments, L¹ is —N(H)—(CH₂)—. In some embodiments, L¹ is —S—(CH₂)—. In some embodiments, L¹ is —S(═O)—(CH₂)—. In some embodiments, L¹ is —S(═O)₂—(CH₂)—. In some embodiments, L¹ is —S(═O)(═NH)—(CH₂)—.

In some embodiments of a compound of Formula (I), (Ia), (Ib), (II), (IIa), (IIb), (IIIa), (IIIb), (IIIc), or (IIId), In some embodiments, R⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R¹⁰ groups, wherein: each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃.

In some embodiments of a compound of Formula (I), (Ia), (Ib), (II), (IIa), (IIb), (IIIa), (IIIb), (IIIc), or (IIId), R⁷ is 4- to 8-membered heterocycloalkyl, where the 4- to 8-membered heterocycloalkyl is unsubstituted or substituted by 1, 2, or 3 R¹⁰ groups, wherein: each R¹⁰ is independently selected from: halogen, —OR⁸, —N(R⁸)₂, —NHSO₂R⁸, —CH₂CN, C₁-C₄ alkyl, —C(═O)—C₁-C₄ hydroxyalkyl, C₁-C₄ hydroxyalkyl, and C₁-C₄ methoxyalkyl; and each R⁸ is independently hydrogen, C₁-C₄ alkyl, —C(═O)—C₁-C₄ alkyl, or 4- to 6-membered heterocycloalkyl, wherein the alkyl is unsubstituted or substituted by 1, 2, or 3 groups independently selected from —F, —CN, —OH, —CO₂H, —C(═NH)NH₂, and 5-membered monocyclic heteroaryl which is unsubstituted or substituted by 1 —CONH₂ group.

In some embodiments of a compound of Formula (I), (Ia), (Ib), (II), (IIa), (IIb), (IIIa), (IIIb), (IIIc), or (IIId), R⁷ is 4- to 8-membered heterocycloalkyl, where the 4- to 8-membered heterocycloalkyl is unsubstituted or substituted by 1, 2, or 3 R¹⁰ groups, wherein: each R¹⁰ is independently selected from: halogen, —OH, —OMe, —N(R⁸)₂, —NHSO₂R⁸, and —CH₂CN; and each R⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl. In some embodiments, R⁷ is 4- to 8-membered heterocycloalkyl, where the 4- to 8-membered heterocycloalkyl is unsubstituted or substituted by 1, 2, or 3 R¹⁰ groups, wherein: each R¹⁰ is independently selected from: halogen, —OH, —OR⁸, —N(R⁸)₂, and —CH₂CN; and each R⁸ is independently hydrogen, C₁-C₂ alkyl, or —C(═O)—C₁-C₂ alkyl. In some embodiments, R⁷ is 4- to 8-membered heterocycloalkyl, where the 4- to 8-membered heterocycloalkyl is unsubstituted or substituted by 1, 2, or 3 R¹⁰ groups, wherein: each R¹⁰ is independently selected from: halogen, —OH, —OR⁸, —N(R⁸)₂, and —CH₂CN; and each R⁸ is independently hydrogen or C₁-C₂ alkyl.

In some embodiments of a compound of Formula (I), (Ia), (Ib), (II), (IIa), (IIb), (IIIa), (IIIb), (IIIc), or (IIId), R⁷ is 4- to 8-membered heterocycloalkyl, where the 4- to 8-membered heterocycloalkyl is unsubstituted or substituted by 1, 2, or 3 R¹⁰ groups, wherein: each R¹⁰ is independently selected from: halogen, —OH, —NH₂, and —CH₂CN. In some embodiments, R⁷ is 4- to 8-membered heterocycloalkyl, where the 4- to 8-membered heterocycloalkyl is unsubstituted or substituted by 1, 2, or 3 R¹⁰ groups, wherein: each R¹⁰ is independently selected from: halogen and —OH. In some embodiments, R⁷ is 4- to 8-membered heterocycloalkyl, where the 4- to 8-membered heterocycloalkyl is unsubstituted or substituted by 1, 2, or 3 R¹⁰ groups, wherein: each R¹⁰ is independently selected from: —F, —Cl, and —OH. In some embodiments, R⁷ is 4- to 8-membered heterocycloalkyl, where the 4- to 8-membered heterocycloalkyl is unsubstituted or substituted by 1, 2, or 3 R¹⁰ groups, wherein: each R¹⁰ is independently selected from: —F and —OH.

In some embodiments, R⁷ is 4- to 6-membered heterocycloalkyl, where the 4- to 6-membered heterocycloalkyl is unsubstituted or substituted by 1, 2, or 3 R¹⁰ groups, wherein: each R¹⁰ is independently selected from: halogen, —OH, —OMe, —N(R⁸)₂, —NHSO₂R⁸, and —CH₂CN; and each R⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl. In some embodiments, R⁷ is 4- to 6-membered heterocycloalkyl, where the 4- to 6-membered heterocycloalkyl is unsubstituted or substituted by 1, 2, or 3 R¹⁰ groups, wherein: each R¹⁰ is independently selected from: halogen, —OH, —OR⁸, —N(R⁸)₂, and —CH₂CN; and each R⁸ is independently hydrogen, C₁-C₂ alkyl, or —C(═O)—C₁-C₂ alkyl. In some embodiments, R⁷ is 4- to 6-membered heterocycloalkyl, where the 4- to 6-membered heterocycloalkyl is unsubstituted or substituted by 1, 2, or 3 R¹⁰ groups, wherein: each R¹⁰ is independently selected from: halogen, —OH, —OR⁸, —N(R⁸)₂, and —CH₂CN; and each R⁸ is independently hydrogen or C₁-C₂ alkyl.

In some embodiments of a compound of Formula (I), (Ia), (Ib), (II), (IIa), (IIb), (IIIa), (IIIb), (IIIc), or (IIId), R⁷ is 4- to 6-membered heterocycloalkyl, where the 4- to 6-membered heterocycloalkyl is unsubstituted or substituted by 1, 2, or 3 R¹⁰ groups, wherein: each R¹⁰ is independently selected from: halogen, —OH, —NH₂, and —CH₂CN. In some embodiments, R⁷ is 4- to 6-membered heterocycloalkyl, where the 4- to 6-membered heterocycloalkyl is unsubstituted or substituted by 1, 2, or 3 R¹⁰ groups, wherein: each R¹⁰ is independently selected from: halogen and —OH. In some embodiments, R⁷ is 4- to 6-membered heterocycloalkyl, where the 4- to 6-membered heterocycloalkyl is unsubstituted or substituted by 1, 2, or 3 R¹⁰ groups, wherein: each R¹⁰ is independently selected from: —F, —Cl, and —OH. In some embodiments, R⁷ is 4- to 6-membered heterocycloalkyl, where the 4- to 6-membered heterocycloalkyl is unsubstituted or substituted by 1, 2, or 3 R¹⁰ groups, wherein: each R¹⁰ is independently selected from: —F and —OH.

In some embodiments of a compound of Formula (I), (Ia), (Ib), (II), (IIa), (IIb), (IIIa), (IIIb), (IIIc), or (IIId), R⁷ is 5- to 6-membered heterocycloalkyl, where the 5- to 6-membered heterocycloalkyl is unsubstituted or substituted by 1, 2, or 3 R¹⁰ groups, wherein: each R¹⁰ is independently selected from: halogen, —OH, —OMe, —N(R⁸)₂, —NHSO₂R⁸, and —CH₂CN; and each R⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl. In some embodiments, R⁷ is 5- to 6-membered heterocycloalkyl, where the 5- to 6-membered heterocycloalkyl is unsubstituted or substituted by 1, 2, or 3 R¹⁰ groups, wherein: each R¹⁰ is independently selected from: halogen, —OH, —OR⁸, —N(R⁸)₂, and —CH₂CN; and each R⁸ is independently hydrogen, C₁-C₂ alkyl, or —C(═O)—C₁-C₂ alkyl. In some embodiments, R⁷ is 5- to 6-membered heterocycloalkyl, where the 5- to 6-membered heterocycloalkyl is unsubstituted or substituted by 1, 2, or 3 R¹⁰ groups, wherein: each R¹⁰ is independently selected from: halogen, —OH, —OR⁸, —N(R⁸)₂, and —CH₂CN; and each R⁸ is independently hydrogen or C₁-C₂ alkyl.

In some embodiments of a compound of Formula (I), (Ia), (Ib), (II), (IIa), (IIb), (IIIa), (IIIb), (IIIc), or (IIId), R⁷ is 5- to 6-membered heterocycloalkyl, where the 5- to 6-membered heterocycloalkyl is unsubstituted or substituted by 1, 2, or 3 R¹⁰ groups, wherein: each R¹⁰ is independently selected from: halogen, —OH, —NH₂, and —CH₂CN. In some embodiments, R⁷ is 5- to 6-membered heterocycloalkyl, where the 5- to 6-membered heterocycloalkyl is unsubstituted or substituted by 1, 2, or 3 R¹⁰ groups, wherein: each R¹⁰ is independently selected from: halogen and —OH. In some embodiments, R⁷ is 5- to 6-membered heterocycloalkyl, where the 5- to 6-membered heterocycloalkyl is unsubstituted or substituted by 1, 2, or 3 R¹⁰ groups, wherein: each R¹⁰ is independently selected from: —F, —Cl, and —OH. In some embodiments, R⁷ is 5- to 6-membered heterocycloalkyl, where the 5- to 6-membered heterocycloalkyl is unsubstituted or substituted by 1, 2, or 3 R¹⁰ groups, wherein: each R¹⁰ is independently selected from: —F and —OH.

In some embodiments, R⁷ is 5-membered heterocycloalkyl, where the 5-membered heterocycloalkyl is unsubstituted or substituted by 1, 2, or 3 R¹⁰ groups, wherein: each R¹⁰ is independently selected from: halogen, —OH, —OMe, —N(R⁸)₂, —NHSO₂R⁸, and —CH₂CN; and each R⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl. In some embodiments, R⁷ is 5-membered heterocycloalkyl, where the 5-membered heterocycloalkyl is unsubstituted or substituted by 1, 2, or 3 R¹⁰ groups, wherein: each R¹⁰ is independently selected from: halogen, —OH, —OR⁸, —N(R⁸)₂, and —CH₂CN; and each R⁸ is independently hydrogen, C₁-C₂ alkyl, or —C(═O)—C₁-C₂ alkyl. In some embodiments, R⁷ is 5-membered heterocycloalkyl, where the 5-membered heterocycloalkyl is unsubstituted or substituted by 1, 2, or 3 R¹⁰ groups, wherein: each R¹⁰ is independently selected from: halogen, —OH, —OR⁸, —N(R⁸)₂, and —CH₂CN; and each R⁸ is independently hydrogen or C₁-C₂ alkyl.

In some embodiments, R⁷ is 6-membered heterocycloalkyl, where the 5-membered heterocycloalkyl is unsubstituted or substituted by 1, 2, or 3 R¹⁰ groups, wherein: each R¹⁰ is independently selected from: halogen, —OH, —OMe, —N(R⁸)₂, —NHSO₂R⁸, and —CH₂CN; and each R⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl. In some embodiments, R⁷ is 6-membered heterocycloalkyl, where the 6-membered heterocycloalkyl is unsubstituted or substituted by 1, 2, or 3 R¹⁰ groups, wherein: each R¹⁰ is independently selected from: halogen, —OH, —OR⁸, —N(R⁸)₂, and —CH₂CN; and each R⁸ is independently hydrogen, C₁-C₂ alkyl, or —C(═O)—C₁-C₂ alkyl. In some embodiments, R⁷ is 6-membered heterocycloalkyl, where the 6-membered heterocycloalkyl is unsubstituted or substituted by 1, 2, or 3 R¹⁰ groups, wherein: each R¹⁰ is independently selected from: halogen, —OH, —OR⁸, —N(R⁸)₂, and —CH₂CN; and each R⁸ is independently hydrogen or C₁-C₂ alkyl.

In some embodiments of a compound of Formula (I), (Ia), (Ib), (II), (IIa), (IIb), (IIIa), (IIIb), (IIIc), or (IIId), R⁷ is 5-membered heterocycloalkyl, where the 5-membered heterocycloalkyl is unsubstituted or substituted by 1, 2, or 3 R¹⁰ groups, wherein: each R¹⁰ is independently selected from: halogen, —OH, —NH₂, and —CH₂CN. In some embodiments, R⁷ is 5-membered heterocycloalkyl, where the 5-membered heterocycloalkyl is unsubstituted or substituted by 1, 2, or 3 R¹⁰ groups, wherein: each R¹⁰ is independently selected from: halogen and —OH. In some embodiments, R⁷ is 5-membered heterocycloalkyl, where the 5-membered heterocycloalkyl is unsubstituted or substituted by 1, 2, or 3 R¹⁰ groups, wherein: each R¹⁰ is independently selected from: —F, —Cl, and —OH. In some embodiments, R⁷ is 5-membered heterocycloalkyl, where the 5-membered heterocycloalkyl is unsubstituted or substituted by 1, 2, or 3 R¹⁰ groups, wherein: each R¹⁰ is independently selected from: —F and —OH.

In some embodiments of a compound of Formula (I), (Ia), (Ib), (II), (IIa), (IIb), (IIIa), (IIIb), (IIIc), or (IIId), R⁷ is 6-membered heterocycloalkyl, where the 6-membered heterocycloalkyl is unsubstituted or substituted by 1, 2, or 3 R¹⁰ groups, wherein: each R¹⁰ is independently selected from: halogen, —OH, —NH₂, and —CH₂CN. In some embodiments, R⁷ is 6-membered heterocycloalkyl, where the 6-membered heterocycloalkyl is unsubstituted or substituted by 1, 2, or 3 R¹⁰ groups, wherein: each R¹⁰ is independently selected from: halogen and —OH. In some embodiments, R⁷ is 6-membered heterocycloalkyl, where the 5-membered heterocycloalkyl is unsubstituted or substituted by 1, 2, or 3 R¹⁰ groups, wherein: each R¹⁰ is independently selected from: —F, —Cl, and —OH. In some embodiments, R⁷ is 6-membered heterocycloalkyl, where the 6-membered heterocycloalkyl is unsubstituted or substituted by 1, 2, or 3 R¹⁰ groups, wherein: each R¹⁰ is independently selected from: —F and —OH.

In some embodiments, R⁷ is tetrahydrofuran, where the tetrahydrofuran is unsubstituted or substituted by 1, 2, or 3 R¹⁰ groups, wherein: each R¹⁰ is independently selected from: halogen, —OH, —OMe, —N(R⁸)₂, —NHSO₂R⁸, and —CH₂CN; and each R⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl. In some embodiments, R⁷ is tetrahydrofuran, where the tetrahydrofuran is unsubstituted or substituted by 1, 2, or 3 R¹⁰ groups, wherein: each R¹⁰ is independently selected from: halogen, —OH, —OR⁸, —N(R⁸)₂, and —CH₂CN; and each R⁸ is independently hydrogen, C₁-C₂ alkyl, or —C(═O)—C₁-C₂ alkyl. In some embodiments, R⁷ is tetrahydrofuran, where the tetrahydrofuran is unsubstituted or substituted by 1, 2, or 3 R¹⁰ groups, wherein: each R¹⁰ is independently selected from: halogen, —OH, —OR⁸, —N(R⁸)₂, and —CH₂CN; and each R⁸ is independently hydrogen or C₁-C₂ alkyl.

In some embodiments, R⁷ is tetrahydropyran, where the tetrahydropyran is unsubstituted or substituted by 1, 2, or 3 R¹⁰ groups, wherein: each R¹⁰ is independently selected from: halogen, —OH, —OMe, —N(R⁸)₂, —NHSO₂R⁸, and —CH₂CN; and each R⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl. In some embodiments, R⁷ is tetrahydropyran, where the tetrahydropyran is unsubstituted or substituted by 1, 2, or 3 R¹⁰ groups, wherein: each R¹⁰ is independently selected from: halogen, —OH, —OR⁸, —N(R⁸)₂, and —CH₂CN; and each R⁸ is independently hydrogen, C₁-C₂ alkyl, or —C(═O)—C₁-C₂ alkyl. In some embodiments, R⁷ is tetrahydropyran, where the tetrahydropyran is unsubstituted or substituted by 1, 2, or 3 R¹⁰ groups, wherein: each R¹⁰ is independently selected from: halogen, —OH, —OR⁸, —N(R⁸)₂, and —CH₂CN; and each R⁸ is independently hydrogen or C₁-C₂ alkyl.

In some embodiments of a compound of Formula (I), (Ia), (Ib), (II), (IIa), (IIb), (IIIa), (IIIb), (IIIc), or (IIId), R⁷ is tetrahydrofuran, where the tetrahydrofuran is unsubstituted or substituted by 1, 2, or 3 R¹⁰ groups, wherein: each R¹⁰ is independently selected from: halogen, —OH, —NH₂, and —CH₂CN. In some embodiments, R⁷ is tetrahydrofuran, where the tetrahydrofuran is unsubstituted or substituted by 1, 2, or 3 R¹⁰ groups, wherein: each R¹⁰ is independently selected from: halogen and —OH. In some embodiments, R⁷ is tetrahydrofuran, where the tetrahydrofuran is unsubstituted or substituted by 1, 2, or 3 R¹⁰ groups, wherein: each R¹⁰ is independently selected from: —F, —Cl, and —OH. In some embodiments, R⁷ is tetrahydrofuran, where the tetrahydrofuran is unsubstituted or substituted by 1, 2, or 3 R¹⁰ groups, wherein: each R¹⁰ is independently selected from: —F and —OH. In some embodiments, R⁷ is tetrahydrofuran substituted by 3 R¹⁰ groups, wherein: each R¹⁰ is independently selected from: —F and —OH.

In some embodiments of a compound of Formula (I), (Ia), (Ib), (II), (IIa), (IIb), (IIIa), (IIIb), (IIIc), or (IIId), R⁷ is tetrahydropyran, where the tetrahydropyran is unsubstituted or substituted by 1, 2, or 3 R¹⁰ groups, wherein: each R¹⁰ is independently selected from: halogen, —OH, —NH₂, and —CH₂CN. In some embodiments, R⁷ is tetrahydropyran, where the tetrahydropyran is unsubstituted or substituted by 1, 2, or 3 R¹⁰ groups, wherein: each R¹⁰ is independently selected from: halogen and —OH. In some embodiments, R⁷ is tetrahydropyran, where the tetrahydropyran is unsubstituted or substituted by 1, 2, or 3 R¹⁰ groups, wherein: each R¹⁰ is independently selected from: —F, —Cl, and —OH. In some embodiments, R⁷ is tetrahydropyran, where the tetrahydropyran is unsubstituted or substituted by 1, 2, or 3 R¹⁰ groups, wherein: each R¹⁰ is independently selected from: —F and —OH. In some embodiments, R⁷ is tetrahydrofuran substituted by 3 R¹⁰ groups, wherein: each R¹⁰ is independently selected from: —F and —OH.

In some embodiments of a compound of Formula (I), (Ia), (Ib), (II), (IIa), (IIb), (IIIa), (IIIb), (IIIc), or (IIId), R⁷ is

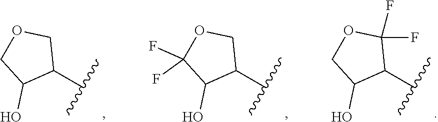 [SMILES: *C1COCC1O ; *C1COC(F)(F)C1O ; *C1C(O)COC1(F)F]

In some embodiments of a compound of Formula (I), (Ia), (Ib), (II), (IIa), (IIb), (IIIa), (IIIb), (IIIc), or (IIId), R⁷ is

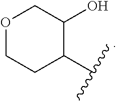 [SMILES: *C1CCOCC1O]

In some embodiments of a compound of Formula (I), (Ia), (Ib), (II), (IIa), (IIb), (IIIa), (IIIb), (IIIc), or (IIId), R⁷ is

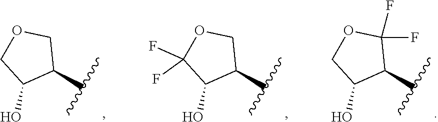 [SMILES: *[C@@H]1COC[C@H]1O ; *[C@@H]1COC(F)(F)[C@H]1O ; *[C@H]1[C@H](O)COC1(F)F]

In some embodiments of a compound of Formula (I), (Ia), (Ib), (II), (IIa), (IIb), (IIIa), (IIIb), (IIIc), or (IIId), R⁷ is

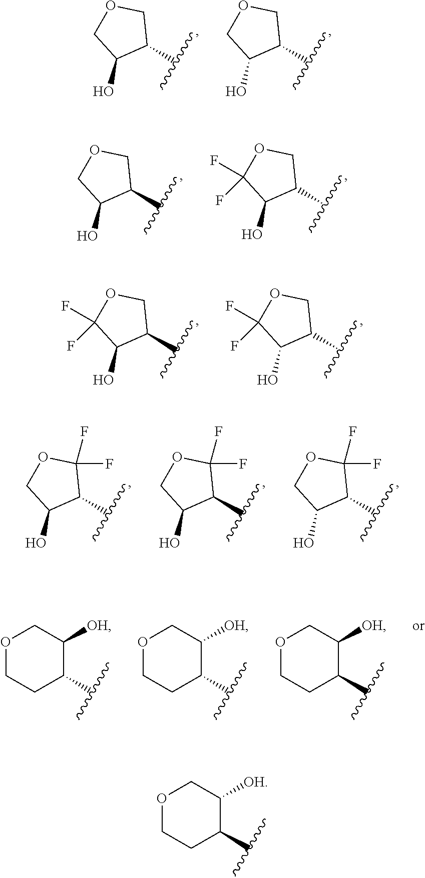

In some embodiments of a compound of Formula (I), (Ia), (Ib), (II), (IIa), (IIb), (IIIa), (IIIb), (IIIc), or (IIId), L¹ is —O—; and R⁷ is tetrahydrofuran, where the tetrahydrofuran is unsubstituted or substituted by 1, 2, or 3 R¹⁰ groups, wherein: each R¹⁰ is independently selected from: halogen, —OH, —NH₂, and —CH₂CN. In some embodiments, L¹ is —O—; and R⁷ is tetrahydrofuran, where the tetrahydrofuran is unsubstituted or substituted by 1, 2, or 3 R¹⁰ groups, wherein: each R¹⁰ is independently selected from: halogen and —OH. In some embodiments, L¹ is —O—; and R⁷ is tetrahydrofuran, where the tetrahydrofuran is unsubstituted or substituted by 1, 2, or 3 R¹⁰ groups, wherein: each R¹⁰ is independently selected from: —F, —Cl, and —OH. In some embodiments, L¹ is —O—; and R⁷ is tetrahydrofuran, where the tetrahydrofuran is unsubstituted or substituted by 1, 2, or 3 R¹⁰ groups, wherein: each R¹⁰ is independently selected from: —F and —OH. In some embodiments, L¹ is —O—; and R⁷ is tetrahydrofuran substituted by 3 R¹⁰ groups, wherein: each R¹⁰ is independently selected from: —F and —OH.

In some embodiments of a compound of Formula (I), (Ia), (Ib), (II), (IIa), (IIb), (IIIa), (IIIb), (IIIc), or (IIId), L¹-R⁷ is

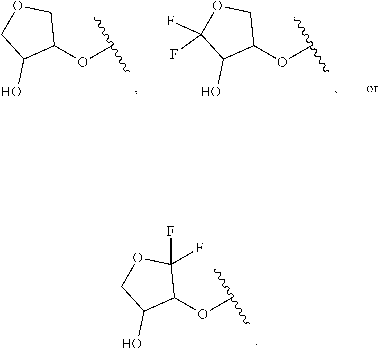 [SMILES: *OC1COCC1O ; *OC1COC(F)(F)C1O ; *OC1C(O)COC1(F)F]

In some embodiments of a compound of Formula (I), (Ia), (Ib), (II), (IIa), (IIb), (IIIa), (IIIb), (IIIc), or (IIId), L¹-R⁷ is

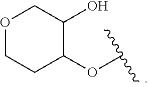 [SMILES: *OC1CCOCC1O]

In some embodiments of a compound of Formula (I), (Ia), (Ib), (II), (IIa), (IIb), (IIIa), (IIIb), (IIIc), or (IIId), L¹-R⁷ is

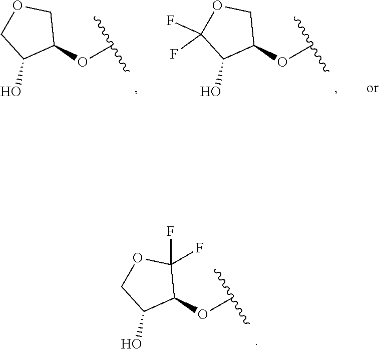 [SMILES: *O[C@@H]1COC[C@H]1O ; *O[C@@H]1COC(F)(F)[C@H]1O ; *O[C@H]1[C@H](O)COC1(F)F]

In some embodiments of a compound of Formula (I), (Ia), (Ib), (II), (IIa), (IIb), (IIIa), (IIIb), (IIIc), or (IIId), L¹-R⁷ is

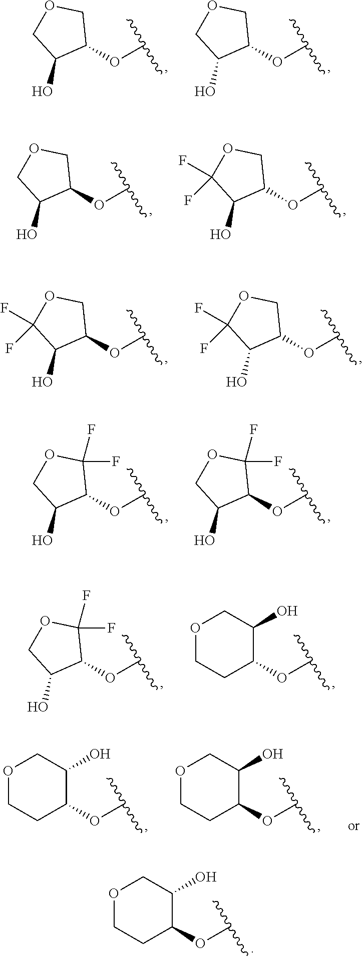

In some embodiments of a compound of Formula (I), (Ia), (Ib), (II), (IIa), (IIb), (IIIa), (IIIb), (IIIc), or (IIId), L¹-R⁷ is

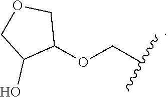 [SMILES: *COC1COCC1O]

In some embodiments of a compound of Formula (I), (Ia), (Ib), (II), (IIa), (IIb), (IIIa), (IIIb), (IIIc), or (IIId), L¹-R⁷ is

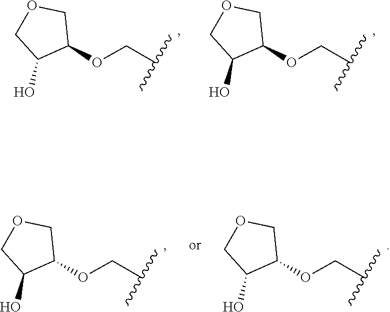 [SMILES: *CO[C@@H]1COC[C@H]1O ; *CO[C@@H]1COC[C@@H]1O ; *CO[C@H]1COC[C@@H]1O ; *CO[C@H]1COC[C@H]1O]

In some embodiments of a compound of Formula (I), (Ia), (Ib), (II), (IIa), (IIb), (IIIa), (IIIb), (IIIc), or (IIId), L¹-R⁷ is

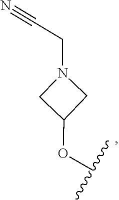 [SMILES: *OC1CN(CC#N)C1]

In some embodiments of a compound of Formula (I), (Ia), (Ib), (II), (IIa), (IIb), (IIIa), (IIIb), (IIIc), or (IIId), L¹ is —NH—; and R⁷ is tetrahydrofuran, where the tetrahydrofuran is unsubstituted or substituted by 1, 2, or 3 R¹⁰ groups, wherein: each R¹⁰ is independently selected from: halogen, —OH, —NH₂, and —CH₂CN. In some embodiments, L¹ is —NH—; and R⁷ is tetrahydrofuran, where the tetrahydrofuran is unsubstituted or substituted by 1, 2, or 3 R¹⁰ groups, wherein: each R¹⁰ is independently selected from: halogen and —OH. In some embodiments, L¹ is —NH—; and R⁷ is tetrahydrofuran, where the tetrahydrofuran is unsubstituted or substituted by 1, 2, or 3 R¹⁰ groups, wherein: each R¹⁰ is independently selected from: —F, —Cl, and —OH. In some embodiments, L¹ is —NH—; and R⁷ is tetrahydrofuran, where the tetrahydrofuran is unsubstituted or substituted by 1, 2, or 3 R¹⁰ groups, wherein: each R¹⁰ is independently selected from: —F and —OH. In some embodiments, L¹ is —O—; and R⁷ is tetrahydrofuran substituted by 3 R¹⁰ groups, wherein: each R¹⁰ is independently selected from: —F and —OH.

In some embodiments of a compound of Formula (I), (Ia), (Ib), (II), (IIa), (IIb), (IIIa), (IIIb), (IIIc), or (IIId), L¹-R⁷ is

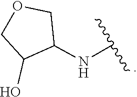 [SMILES: *NC1COCC1O]

In some embodiments of a compound of Formula (I), (Ia), (Ib), (II), (IIa), (IIb), (IIIa), (IIIb), (IIIc), or (IIId), L¹-R⁷ is

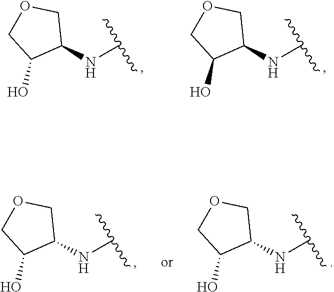 [SMILES: *N[C@@H]1COC[C@H]1O ; *N[C@@H]1COC[C@@H]1O ; *N[C@H]1COC[C@H]1O ; *N[C@H]1COC[C@H]1O]

In some embodiments of a compound of Formula (I), (Ia), (Ib), (II), (IIa), (IIb), (IIIa), (IIIb), (IIIc), or (IIId), R⁷ is a single diastereomer. In some embodiments, R⁷ is a single diastereomer and exhibits improved potency to a comparative compound wherein R⁷ is a different stereoisomer. In some embodiments, R⁷ is a single diastereomer and exhibits improved potency in vitro to a comparative compound wherein R⁷ is a different stereoisomer. In some embodiments, R⁷ is a single diastereomer and exhibits improved potency in vivo to a comparative compound wherein R⁷ is a different stereoisomer.

In some embodiments of a compound of Formula (I), (Ia), (Ib), (II), (IIa), (IIb), (IIIa), (IIIb), (IIIc), or (IIId), R⁷ is a 4- to 6-membered heterocycloalkyl substituted by 1 or 2 R¹⁰ groups, wherein each R¹⁰ is independently selected from: halogen, —OH, —OMe, —N(R⁸)₂, —NHSO₂R⁸, and —CH₂CN. In some embodiments, R⁷ is a 4- to 6-membered heterocycloalkyl substituted by 1 or 2 R¹⁰ groups, wherein each R¹⁰ is independently selected from: halogen, —OH, and —CH₂CN. In some embodiments, R⁷ is a 4- to 6-membered heterocycloalkyl substituted by 1 or 2 R¹⁰ groups, wherein each R¹⁰ is independently selected from: —F, —Cl, —OH, and —CH₂CN. In some embodiments, R⁷ is a 4- to 6-membered heterocycloalkyl substituted by —OH.

In some embodiments, R⁷ is a 1,2-trans-disubstituted 4- to 6-membered heterocycloalkyl. In some embodiments, R⁷ is

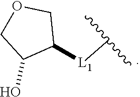

In some embodiments, R⁷ is

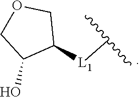

In some embodiments, R⁷ is

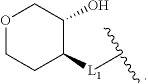

In some embodiments, R⁷ is

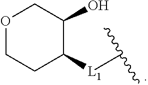

In some embodiments, R⁷ is a 1,2-cis-disubstituted 4- to 6-membered heterocycloalkyl. In some embodiments, R⁷ is

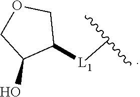

In some embodiments, R⁷ is

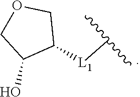

In some embodiments, R⁷ is

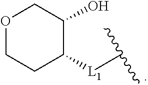

In some embodiments, R⁷ is

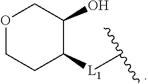

In some embodiments, the compound is a compound of Formula (IIIa), or a pharmaceutically acceptable salt, solvate, or stereoisomer thereof. In some embodiments, the compound is a compound of Formula (IIIb), or a pharmaceutically acceptable salt, solvate, or stereoisomer thereof. In some embodiments, the compound is a compound of Formula (IIIc), or a pharmaceutically acceptable salt, solvate, or stereoisomer thereof. In some embodiments, the compound is a compound of Formula (IIId), or a pharmaceutically acceptable salt, solvate, or stereoisomer thereof.

In some embodiments, the compound of Formula (I) is a compound of Formula (IV):

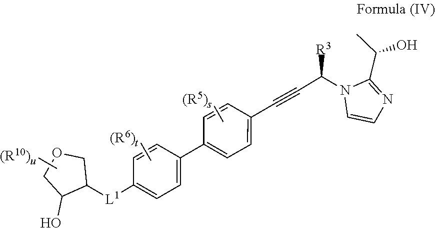

or a pharmaceutically acceptable salt, solvate, or stereoisomer thereof.

In some embodiments of a compound of Formula (IV), R³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂; each R⁵ is halogen or C₁-C₄ alkyl; each R⁶ is halogen or C₁-C₄ alkyl; L¹ is a bond, —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and —S(═O)(═NR⁹)—, wherein: R⁹ is hydrogen or C₁-C₆ alkyl; each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo; s is 0, 1, or 2; t is 0, 1, or 2; u is 0, 1, or 2; and wherein at least one of the following: (i) s is 1 or 2, and at least one R⁵ is halogen; (ii) t is 1 or 2, and at least one R⁶ is halogen; and (iii) u is 0, 1, or 2, and at least one R¹⁰ is halogen.

In some embodiments of a compound of Formula (IV), R³ is hydrogen, —(C₁-C₄ alkylene)-OH, or —(C₁-C₄ alkylene)-NH₂; R⁵ is —F; R⁶ is —F; L¹ is a bond, —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and —S(═O)(═NR⁹)—, wherein: R⁹ is hydrogen or C₁-C₆ alkyl; each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; s is 0, 1, or 2; t is 0, 1, or 2; u is 0, 1, or 2; and wherein at least one of the following: (i) s is 1 or 2, and at least one R⁵ is halogen; (ii) t is 1 or 2, and at least one R⁶ is halogen; and (iii) u is 0, 1, or 2, and at least one R¹⁰ is halogen.

In some embodiments of a compound of Formula (IV), each R⁵ is independently halogen or C₁-C₄ alkyl; and each R⁶ is independently halogen or C₁-C₄ alkyl. In some embodiments, each R⁵ is independently halogen; and each R⁶ is independently halogen. In some embodiments, each R⁵ is independently —F; and each R⁶ is independently —F. In some embodiments, each R⁵ is —F; and t is 0. In some embodiments, R⁵ is —F; and R⁶ is —F.

In some embodiments of a compound of Formula (IV), L¹ is a bond, —(C₁-C₂ alkylene)-, —X¹—, or —X²—(C₁-C₂ alkylene)-, wherein: X¹ and X² are each selected from: —O—, —N(R⁹)—, —S—, —S(═O)—, —S(═O)₂—, and —S(═O)(═NR⁹)—, wherein: R⁹ is hydrogen or C₁-C₆ alkyl. In some embodiments, L¹ is a bond, —(C₁-C₂ alkylene)-, —X¹—, or —X²—(C₁-C₂ alkylene)-, wherein: X¹ and X² are each selected from: —O—, —N(H)—, —S—, —S(═O)—, —S(═O)₂—, and —S(═O)(═NH)—. In some embodiments, L¹ is a bond, —(CH₂)—, —X¹—, or —X²—(CH₂)—, wherein: X¹ and X² are each selected from: —O—, —N(R⁹)—, —S—, —S(═O)—, —S(═O)₂—, and —S(═O)(═NR⁹)—, wherein: R⁹ is hydrogen or C₁-C₆ alkyl. In some embodiments, L¹ is a bond, —(CH₂)—, —X¹—, or —X²—(CH₂)—, wherein: X¹ and X² are each selected from: —O—, —N(H)—, —S—, —S(═O)—, —S(═O)₂—, and —S(═O)(═NH)—. In some embodiments, L¹ is a bond, —(CH₂)—, —X¹—, or —X²—(CH₂)—, wherein: X¹ and X² are each selected from: —O—, —N(R⁹)—, and —S—. In some embodiments, L¹ is a bond, —(CH₂)—, —X¹—, or —X²—(CH₂)—, wherein: X¹ and X² are each selected from: —O—. In some embodiments, L¹ is —O—.

In some embodiments, the compound of Formula (I) is a compound of Formula (V):

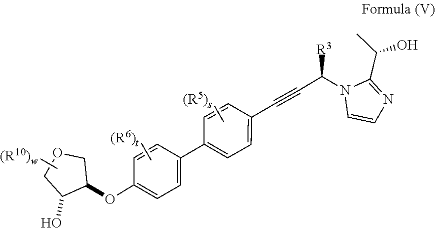

or a pharmaceutically acceptable salt, solvate, or stereoisomer thereof.

In some embodiments of a compound of Formula (V), R³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂; each R⁵ is halogen or C₁-C₄ alkyl; each R⁶ is halogen or C₁-C₄ alkyl; L¹ is a bond, —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and —S(═O)(═NR⁹)—, wherein: R⁹ is hydrogen or C₁-C₆ alkyl; each R¹⁰ is independently selected from: halogen, —OR⁸, —N(R⁸)₂, —CO₂R⁸, —CON(R¹)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo; s is 0, 1, or 2; t is 0, 1, or 2; u is 0, 1, or 2; and wherein at least one of the following: (i) s is 1 or 2, and at least one R⁵ is halogen; (ii) t is 1 or 2, and at least one R⁶ is halogen; and (iii) u is 0, 1, or 2, and at least one R¹⁰ is halogen.

In some embodiments of a compound of Formula (V), R³ is hydrogen, —(C₁-C₄ alkylene)-OH, or —(C₁-C₄ alkylene)-NH₂; R⁵ is —F; R⁶ is —F; L¹ is a bond, —(C₁-C₆ alkylene)-, —X¹—, or —X²—(C₁-C₆ alkylene)-, wherein: X¹ and X² are each selected from: —O—, —N(R⁹)—, —S—, —S(═O)—, —S(═O)₂—, and —S(═O)(═NR⁹)—, wherein: R⁹ is hydrogen or C₁-C₆ alkyl; each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; s is 0, 1, or 2; t is 0, 1, or 2; u is 0, 1, or 2; and wherein at least one of the following: (i) s is 1 or 2, and at least one R⁵ is halogen; (ii) t is 1 or 2, and at least one R⁶ is halogen; and (iii) u is 0, 1, or 2, and at least one R¹⁰ is halogen.

In some embodiments of a compound of Formula (V), each R⁵ is independently halogen or C₁-C₄ alkyl; and each R⁶ is independently halogen or C₁-C₄ alkyl. In some embodiments, each R⁵ is independently halogen; and each R⁶ is independently halogen. In some embodiments, each R⁵ is independently —F; and each R⁶ is independently —F. In some embodiments, each R⁵ is —F; and t is 0. In some embodiments, R⁵ is —F; and R⁶ is —F.

In some embodiments of a compound of Formula (IV) or (V), u is 0. In some embodiments, u is 1. In some embodiments, u is 2.

In some embodiments of a compound of Formula (IV) or (V), s is 0, 1, or 2; and u is 0. In some embodiments, s is 0, 1 or 2; and u is 1. In some embodiments, s is 0, 1 or 2; and u is 2. In some embodiments, s is 0 and u is 0. In some embodiments, s is 1 and u is 0. In some embodiments, s is 2 and u is 0. In some embodiments, s is 0 and u is 1. In some embodiments, s is 1 and u is 1. In some embodiments, s is 2 and t is 1. In some embodiments, s is 0 and u is 2. In some embodiments, s is 1 and u is 2. In some embodiments, s is 2 and u is 2.

In some embodiments of a compound, salt, solvate, or stereoisomer of Formula (V), t is 0, 1, or 2; and u is 0. In some embodiments, t is 0, 1, or 2; and u is 1. In some embodiments, t is 0, 1, or 2; and u is 2. In some embodiments, tis 0 and u is 0. In some embodiments, tis 1 and u is 0. In some embodiments, t is 2 and u is 0. In some embodiments, t is 0 and u is 1. In some embodiments, t is 1 and u is 1. In some embodiments, t is 2 and u is 1. In some embodiments, t is 0 and u is 2. In some embodiments, t is 1 and u is 2. In some embodiments, t is 2 and u is 2.

In some embodiments of a compound, salt, solvate, or stereoisomer of Formula (V), each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo.

In some embodiments of a compound, salt, solvate, or stereoisomer of Formula (V), each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN and C₁-C₄ alkyl, further wherein: each R⁸ is independently hydrogen or C₁-C₄ alkyl. In some embodiments, each R¹⁰ is independently selected from: halogen, —OR⁸, —N(R⁸)₂, —CO₂R⁸, —CON(R⁸)₂, —CH₂CN, and C₁-C₄ alkyl, further wherein: each R⁸ is independently hydrogen or C₁-C₄ alkyl. In some embodiments, each R¹⁰ is independently selected from: halogen, —OR⁸, —N(R⁸)₂, —CH₂CN, and C₁-C₄ alkyl, further wherein: each R⁸ is independently hydrogen or C₁-C₄ alkyl. In some embodiments, each R¹⁰ is independently selected from: halogen, —OH, —NH₂, —CO₂H, —CONH₂, —CH₂CN, and C₁-C₄ alkyl. In some embodiments, each R¹⁰ is independently selected from: halogen, —OH, —NH₂, —CH₂CN, and C₁-C₄ alkyl. In some embodiments, each R¹⁰ is independently selected from: halogen, —OH, —NH₂, and C₁-C₄ alkyl. In some embodiments, each R¹⁰ is independently selected from: —F, —Cl, —OH, —NH₂, and C₁-C₄ alkyl. In some embodiments, each R¹⁰ is independently selected from: —F, —Cl, —OH, —NH₂, and methyl. In some embodiments, each R¹⁰ is independently selected from: —F, —Cl, —OH, and —NH₂. In some embodiments, each R¹⁰ is independently selected from: —F, —OH, and —NH₂. In some embodiments, each R¹⁰ is independently selected from: —F and —OH.

In some embodiments of a compound of Formula (I), (Ia), (Ib), (II), (IIa), (IIb), (IIIa), (IIIb), (IIIc), (IIId), (IV), or (V), R³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂. In some embodiments, R³ is —(C₁-C₄ alkylene)-OH, —(C₁-C₄ alkylene)-NH₂, —(C₃-C₆ cycloalkylene)-OH, or —(C₃-C₆ cycloalkylene)-NH₂. In some embodiments, R³ is —(C₁-C₄ alkylene)-OH and —(C₃-C₆ cycloalkylene)-OH. In some embodiments, R³ is —(C₁-C₄ alkylene)-NH₂ or —(C₃-C₆ cycloalkylene)-OH.

In some embodiments of a compound of Formula (I), (Ia), (Ib), (II), (IIa), (IIb), (IIIa), (IIIb), (IIIc), (IIId), (IV), or (V), R³ is hydrogen, —(C₁-C₄ alkylene)-OH, —(C₁-C₄ alkylene)-NH₂, -(cyclopropylene)-OH, or -(cyclopropylene)-NH₂. In some embodiments, R³ is —(C₁-C₄ alkylene)-OH, —(C₁-C₄ alkylene)-NH₂, -(cyclopropylene)-OH, or -(cyclopropylene)-NH₂. In some embodiments, R³ is —(C₁-C₄ alkylene)-OH and -(cyclopropylene)-OH. In some embodiments, R³ is —(C₁-C₄ alkylene)-NH₂ or -(cyclopropylene)-OH.

In some embodiments of a compound of Formula (I), (Ia), (Ib), (II), (IIa), (IIb), (IIIa), (IIIb), (IIIc), (IIId), (IV), or (V), R³ is hydrogen. In some embodiments, R³ is —(C₁-C₄ alkylene)-OH. In some embodiments, R³ is —(C₁-C₂ alkylene)-OH.

In some embodiments, R³ is —CH₂OH, —CH₂CH₂OH, —CH₂CH₂CH₂OH, —CH₂CH₂CH₂CH₂OH, —CH(CH₃)OH, —CH₂CH(CH₃)OH, —CH(CH₃)CH₂OH, —CH₂CH(CH₂CH₃)OH, or —CH(CH₂CH₃)CH₂OH. In some embodiments, R³ is —CH₂OH, —CH₂CH₂OH, or —CH(CH₃)OH. In some embodiments, R³ is —CH₂OH or —CH₂CH₂OH. In some embodiments, R³ is —CH₂OH. In some embodiments, R³ is —CH₂CH₂OH.

In some embodiments of a compound of Formula (I), (Ia), (Ib), (II), (IIa), (IIb), (IIIa), (IIIb), (IIIc), (IIId), (IV), or (V), R³ is —(C₁-C₄ alkylene)-NH₂. In some embodiments, R³ is —(C₁-C₂ alkylene)-NH₂. In some embodiments, R³ is —CH₂NH₂, —CH₂CH₂NH₂, —CH₂CH₂CH₂NH₂, —CH₂CH₂CH₂CH₂NH₂, —CH(CH₃)NH₂, —CH₂CH(CH₃)NH₂, —CH(CH₃)CH₂NH₂, —CH₂CH(CH₂CH₃)NH₂, or —CH(CH₂CH₃)CH₂NH₂. In some embodiments, R³ is —CH₂NH₂, —CH₂CH₂NH₂, or —CH(CH₃)NH₂. In some embodiments, R³ is —CH₂NH₂ or —CH₂CH₂NH₂. In some embodiments, R³ is —CH₂NH₂.

In some embodiments of a compound of Formula (I), (Ia), (Ib), (II), (IIa), (IIb), (IIIa), (IIIb), (IIIc), (IIId), (IV), or (V), R³ is hydrogen or —(C₁-C₄ alkylene)-OH. In some embodiments, R³ is hydrogen or —(C₁-C₂ alkylene)-OH. In some embodiments, R³ is hydrogen, —CH₂OH, —CH₂CH₂OH, —CH₂CH₂CH₂OH, —CH₂CH₂CH₂CH₂OH, —CH(CH₃)OH, —CH₂CH(CH₃)OH, —CH(CH₃)CH₂OH, —CH₂CH(CH₂CH₃)OH, or —CH(CH₂CH₃)CH₂OH. In some embodiments, R³ is hydrogen, —CH₂OH, —CH₂CH₂OH, or —CH(CH₃)OH. In some embodiments, R³ is hydrogen, —CH₂OH or —CH₂CH₂OH. In some embodiments, R³ is hydrogen or —CH₂OH.

In some embodiments of a compound of Formula (I), (Ia), (Ib), (II), (IIa), (IIb), (IIIa), (IIIb), (IIIc), (IIId), (IV), or (V), R³ is hydrogen, —(C₁-C₂ alkylene)-OH, or —(C₁-C₂ alkylene)-NH₂. In some embodiments, R³ is hydrogen, —CH₂OH, —CH₂CH₂OH, or —CH₂NH₂. In some embodiments, R³ is hydrogen, —CH₂OH, or —CH₂NH₂.

In some embodiments of a compound of Formula (I), (Ia), (Ib), (II), (IIa), (IIb), (IIIa), (IIIb), (IIIc), (IIId), (IV), or (V), —(C₃-C₆ cycloalkylene)-OH or —(C₃-C₆ cycloalkylene)-NH₂. In some embodiments, R³ is —(C₃-C₅ cycloalkylene)-OH or —(C₃-C₅ cycloalkylene)-NH₂. In some embodiments, R³ is —(C₃-C₄ cycloalkylene)-OH or —(C₃-C₄ cycloalkylene)-NH₂. In some embodiments, R³ is -(cyclopropylene)-OH or -(cyclopropylene)-NH₂. In some embodiments, R³ is

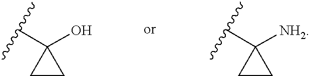 [SMILES: *C1(O)CC1 ; "or" ; *C1(N)CC1]

In some embodiments of a compound of Formula (I), (Ia), (Ib), (II), (IIa), (IIb), (IIIa), (IIIb), (IIIc), (IIId), (IV), or (V), R³ is C₃-C₆ cycloalkylene)-OH. In some embodiments, —(C₃-C₅ cycloalkylene)-OH. In some embodiments, R³ is —(C₃-C₄ cycloalkylene)-OH. In some embodiments, R³ is -(cyclopropylene)-OH. In some embodiments, R³ is

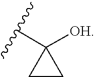 [SMILES: *C1(O)CC1]

In some embodiments of a compound of Formula (I), (Ia), (Ib), (II), (IIa), (IIb), (IIIa), (IIIb), (IIIc), (IIId), (IV), or (V), R³ is —(C₃-C₆ cycloalkylene)-NH₂. In some embodiments, R³ is —(C₃-C₅ cycloalkylene)-NH₂. In some embodiments, R³ is —(C₃-C₄ cycloalkylene)-NH₂. In some embodiments, R³ is -(cyclopropylene)-NH₂. In some embodiments, R³ is

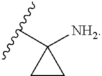 [SMILES: *C1(N)CC1]

In some embodiments of a compound, salt, solvate, or stereoisomer of Formula (I), (Ia), (Ib), (II), (IIa), (IIb), (IIIa), (IIIb), (IIIc), (IIId), (IV), or (V), each R⁵ and R⁶ is independently halogen, or unsubstituted C₁-C₄ alkyl. In some embodiments, each R⁵ and R⁶ is independently —F, —Cl, —Br, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, each R⁵ and R⁶ is independently —F, —Cl, —CH₃, —CH₂CH₃, or —CH(CH₃)₂. In some embodiments, each R⁵ and R⁶ is independently —F, —Cl, or —CH₃.

In some embodiments of a compound, salt, solvate, or stereoisomer of Formula (I), (Ia), (Ib), (II), (IIa), (IIb), (IIIa), (IIIb), (IIIc), (IIId), (IV), or (V), each R⁵ is independently halogen, or unsubstituted C₁-C₄ alkyl. In some embodiments, each R⁵ is independently —F, —Cl, —Br, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, each R⁵ is independently —F, —Cl, —CH₃, —CH₂CH₃, or —CH(CH₃)₂. In some embodiments, each R⁵ is independently —F, —Cl, or —CH₃.

In some embodiments of a compound, salt, solvate, or stereoisomer of Formula (I), (Ia), (Ib), (II), (IIa), (IIb), (IIIa), (IIIb), (IIIc), (IIId), (IV), or (V), each R⁶ is independently halogen, or unsubstituted C₁-C₄ alkyl. In some embodiments, each R⁶ is independently —F, —Cl, —Br, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, each R⁶ is independently —F, —Cl, —CH₃, —CH₂CH₃, or —CH(CH₃)₂. In some embodiments, each R⁶ is independently —F, —Cl, or —CH₃.

In some embodiments of a compound, salt, solvate, or stereoisomer of Formula (I), (Ia), (Ib), (II), (IIa), (IIb), (IIIa), (IIIb), (IIIc), (IIId), (IV), or (V), each R⁵ is independently —F, —Cl, —Br, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃; and t is 0. In some embodiments, each R⁵ is independently —F, —Cl, —CH₃, —CH₂CH₃, or —CH(CH₃)₂; and t is 0. In some embodiments, each R⁵ is independently —F, —Cl, or —CH₃; and t is 0.

In some embodiments of a compound, salt, solvate, or stereoisomer of Formula (I), (Ia), (Ib), (II), (IIa), (IIb), (IIIa), (IIIb), (IIIc), (IIId), (IV), or (V), s is 0; and each R⁶ is independently —F, —Cl, —Br, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, s is 0; and each R⁶ is independently —F, —Cl, —CH₃, —CH₂CH₃, or —CH(CH₃)₂. In some embodiments, s is 0; and each R⁶ is independently —F, —Cl, or —CH₃.

In some embodiments of a compound, salt, solvate, or stereoisomer of Formula (I), (Ia), (Ib), (II), (IIa), (IIb), (IIIa), (IIIb), (IIIc), (IIId), (IV), or (V), s is 0. In some embodiments, s is 1. In some embodiments, s is 2.

In some embodiments of a compound, salt, solvate, or stereoisomer of Formula (I), (Ia), (Ib), (II), (IIa), (IIb), (IIIa), (IIIb), (IIIc), (IIId), (IV), or (V), t is 0. In some embodiments, t is 1. In some embodiments, t is 2.

In some embodiments of a compound of Formula (I), (Ia), (Ib), (II), (IIa), (IIb), (IIIa), (IIIb), (IIIc), (IIId), (IV), or (V), s is 0, 1, or 2; and t is 0. In some embodiments, s is 0, 1 or 2; and t is 1. In some embodiments, s is 0, 1 or 2; and t is 2. In some embodiments, s is 0 and t is 0. In some embodiments, s is 1 and t is 0. In some embodiments, s is 2 and t is 0. In some embodiments, s is 0 and t is 1. In some embodiments, s is 1 and t is 1. In some embodiments, s is 2 and t is 1. In some embodiments, s is 0 and t is 2. In some embodiments, s is 1 and t is 2. In some embodiments, s is 2 and t is 2.

In some embodiments of a compound, salt, solvate, or stereoisomer of Formula (I), (Ia), (Ib), (II), (IIa), (IIb), (IIIa), (IIIb), (IIIc), (IIId), (IV), or (V), t is 0, 1, or 2; and s is 0. In some embodiments, tis 0, 1, or 2; and s is 1. In some embodiments, tis 0, 1, or 2; and s is 2. In some embodiments, tis 0 and s is 0. In some embodiments, t is 1 and s is 0. In some embodiments, t is 2 and s is 0. In some embodiments, t is 0 and s is 1. In some embodiments, tis 1 and s is 1. In some embodiments, tis 2 and s is 1. In some embodiments, tis 0 and s is 2. In some embodiments, t is 1 and s is 2. In some embodiments, t is 2 and s is 2.

In some embodiments of a compound, salt, solvate, or stereoisomer of Formula (I), (Ia), (Ib), (II), (IIa), (IIb), (IIIa), (IIIb), (IIIc), (IIId), (IV), or (V), at least one of the following: (i) s is 1 or 2, and at least one R⁵ is halogen; (ii) t is 1 or 2, and at least one R⁶ is halogen; and (iii) the alkyl, heteroalkyl, cycloalkyl, or heterocycloalkyl of R⁷ is substituted by 1, 2, or 3 R¹⁰ groups, and at least one R¹⁰ is halogen. In some embodiments, at least one of the following: (i) s is 1 or 2, and at least one R⁵ is halogen; (ii) t is 1 or 2, and at least one R⁶ is halogen. In some embodiments, at least one of the following: (i) s is 1 or 2, and at least one R⁵ is halogen; and (iii) the alkyl, heteroalkyl, cycloalkyl, or heterocycloalkyl of R⁷ is substituted by 1, 2, or 3 R¹⁰ groups, and at least one R¹⁰ is halogen. In some embodiments, at least one of the following: (ii) t is 1 or 2, and at least one R⁶ is halogen; and (iii) the alkyl, heteroalkyl, cycloalkyl, or heterocycloalkyl of R⁷ is substituted by 1, 2, or 3 R¹⁰ groups, and at least one R¹⁰ is halogen. In some embodiments, s is 1 or 2, and at least one R⁵ is halogen. In some embodiments, tis 1 or 2, and at least one R⁶ is halogen. In some embodiments, the alkyl, heteroalkyl, cycloalkyl, or heterocycloalkyl of R⁷ is substituted by 1, 2, or 3 R¹⁰ groups, and at least one R¹⁰ is halogen.

In some embodiments of a compound, salt, solvate, or stereoisomer of Formula (I), (Ia), (Ib), (II), (IIa), (IIb), (IIIa), (IIIb), (IIIc), (IIId), (IV), or (V), at least one of the following: (i) s is 1 or 2, and R⁵ is halogen; (ii) t is 1 or 2, and R⁶ is halogen; and (iii) the alkyl, heteroalkyl, cycloalkyl, or heterocycloalkyl of R⁷ is substituted by 1, 2, or 3 R¹⁰ groups, and R¹⁰ is halogen. In some embodiments, at least one of the following: (i) s is 1 or 2, and R⁵ is halogen; and (ii) t is 1 or 2, and R⁶ is halogen. In some embodiments, at least one of the following: (i) s is 1 or 2, and R⁵ is halogen; and (iii) the alkyl, heteroalkyl, cycloalkyl, or heterocycloalkyl of R⁷ is substituted by 1, 2, or 3 R¹⁰ groups, and R¹⁰ is halogen. In some embodiments, at least one of the following: (ii) t is 1 or 2, and R⁶ is halogen; and (iii) the alkyl, heteroalkyl, cycloalkyl, or heterocycloalkyl of R⁷ is substituted by 1, 2, or 3 R¹⁰ groups, and R¹⁰ is halogen. In some embodiments, s is 1 or 2, and R⁵ is halogen. In some embodiments, tis 1 or 2, and R⁶ is halogen. In some embodiments, the alkyl, heteroalkyl, cycloalkyl, or heterocycloalkyl of R⁷ is substituted by 1, 2, or 3 R¹⁰ groups, and R¹⁰ is halogen.

In some embodiments of a compound, salt, solvate, or stereoisomer of Formula (I), (Ia), (Ib), (II), (IIa), (IIb), (IIIa), (IIIb), (IIIc), (IIId), (IV), or (V), at least one of the following: (i) s is 1 or 2, and at least one R⁵ is —F; (ii) t is 1 or 2, and at least one R⁶ is —F; and (iii) the alkyl, heteroalkyl, cycloalkyl, or heterocycloalkyl of R⁷ is substituted by 1, 2, or 3 R¹⁰ groups, and at least one R¹⁰ is —F. In some embodiments, at least one of the following: (i) s is 1 or 2, and at least one R⁵ is —F; and (ii) t is 1 or 2, and at least one R⁶ is —F. In some embodiments, at least one of the following: (i) s is 1 or 2, and at least one R⁵ is —F; and (iii) the alkyl, heteroalkyl, cycloalkyl, or heterocycloalkyl of R⁷ is substituted by 1, 2, or 3 R¹⁰ groups, and at least one R¹⁰ is —F. In some embodiments, at least one of the following: (ii) t is 1 or 2, and at least one R⁶ is —F; and (iii) the alkyl, heteroalkyl, cycloalkyl, or heterocycloalkyl of R⁷ is substituted by 1, 2, or 3 R¹⁰ groups, and at least one R¹⁰ is —F. In some embodiments, s is 1 or 2, and at least one R⁵ is —F. In some embodiments, t is 1 or 2, and at least one R⁶ is —F. In some embodiments, the alkyl, heteroalkyl, cycloalkyl, or heterocycloalkyl of R⁷ is substituted by 1, 2, or 3 R¹⁰ groups, and at least one R¹⁰ is —F.

In some embodiments of a compound, salt, solvate, or stereoisomer of Formula (I), (Ia), (Ib), (II), (IIa), (IIb), (IIIa), (IIIb), (IIIc), (IIId), (IV), or (V), at least one of the following: (i) s is 1 or 2, and R⁵ is —F; (ii) t is 1 or 2, and R⁶ is —F; and (iii) the alkyl, heteroalkyl, cycloalkyl, or heterocycloalkyl of R⁷ is substituted by 1, 2, or 3 R¹⁰ groups, and R¹⁰ is —F. In some embodiments, at least one of the following: (i) s is 1 or 2, and R⁵ is —F; and (ii) t is 1 or 2, and R⁶ is —F. In some embodiments, at least one of the following: (i) s is 1 or 2, and R⁵ is —F; and (iii) the alkyl, heteroalkyl, cycloalkyl, or heterocycloalkyl of R⁷ is substituted by 1, 2, or 3 R¹⁰ groups, and R¹⁰ is —F. In some embodiments, at least one of the following: (ii) t is 1 or 2, and R⁶ is —F; and (iii) the alkyl, heteroalkyl, cycloalkyl, or heterocycloalkyl of R⁷ is substituted by 1, 2, or 3 R¹⁰ groups, and R¹⁰ is —F. In some embodiments, s is 1 or 2, and R⁵ is —F. In some embodiments, t is 1 or 2, and R⁶ is —F. In some embodiments, the alkyl, heteroalkyl, cycloalkyl, or heterocycloalkyl of R⁷ is substituted by 1, 2, or 3 R¹⁰ groups, and R¹⁰ is —F.

In some embodiments of a compound, salt, solvate, or stereoisomer of Formula (I), (Ia), (Ib), (II), (IIa), (IIb), (IIIa), (IIIb), (IIIc), (IIId), (IV), or (V), at least one R⁵, R⁶, or R¹⁰ is —F. In some embodiments, at least one R⁵ or R⁶ is —F. In some embodiments, at least one R⁵ or R¹⁰ is —F. In some embodiments, at least one R¹⁰ or R⁶ is —F. In some embodiments, at least one R⁵ is —F. In some embodiments, at least one R⁶ is —F. In some embodiments, at least one R¹⁰ is —F. In some embodiments, at least two R⁵, R⁶, or R¹⁰ is —F. In some embodiments, at least two R⁵ or R⁶ is —F. In some embodiments, at least two R⁵ or R¹⁰ is —F. In some embodiments, at least two R¹⁰ or R⁶ is —F. In some embodiments, at least two R⁵ is —F. In some embodiments, at least two R⁶ is —F. In some embodiments, at least two R¹⁰ is —F.

In some embodiments of a compound, salt, solvate, or stereoisomer of Formula (I), (Ia), (Ib), (II), (IIa), (IIb), (IIIa), (IIIb), (IIIc), (IIId), (IV), or (V), at least one R⁵, R⁶, or R¹⁰ is —Cl. In some embodiments, at least one R⁵ or R⁶ is —Cl. In some embodiments, at least one R⁵ or R¹⁰ is —Cl. In some embodiments, at least one R¹⁰ or R⁶ is —Cl. In some embodiments, at least one R⁵ is —Cl. In some embodiments, at least one R⁶ is —Cl. In some embodiments, at least one R¹⁰ is —Cl. In some embodiments, at least two R⁵, R⁶, or R¹⁰ is —Cl. In some embodiments, at least two R⁵ or R⁶ is —F. In some embodiments, at least two R⁵ or R¹⁰ is —Cl. In some embodiments, at least two R¹⁰ or R⁶ is —Cl. In some embodiments, at least two R⁵ is —Cl. In some embodiments, at least two R⁶ is —Cl. In some embodiments, at least two R¹⁰ is —Cl.

In some embodiments, the compound is selected from:

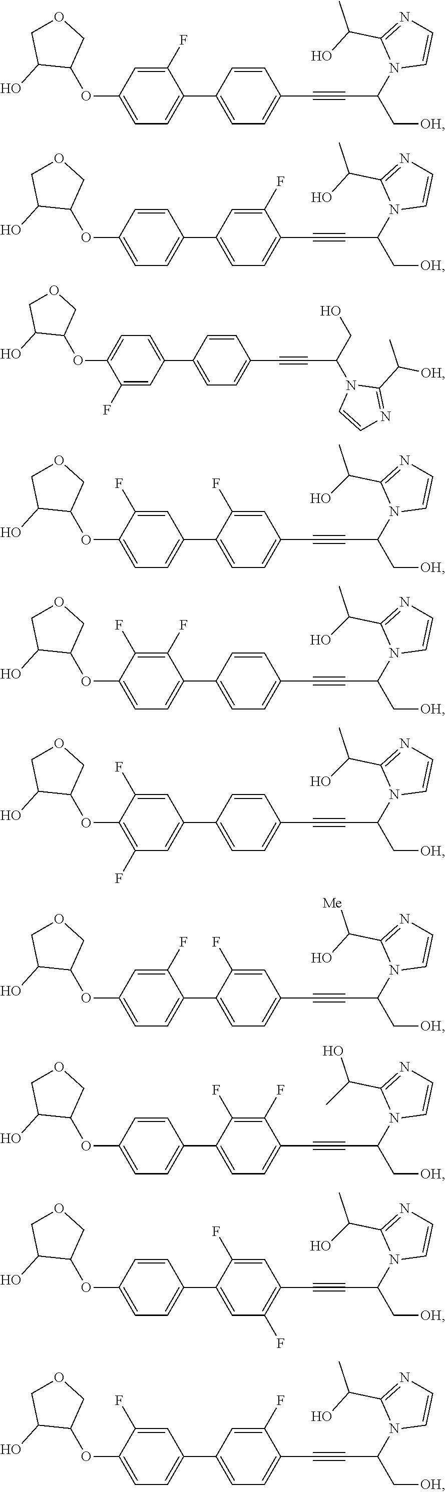

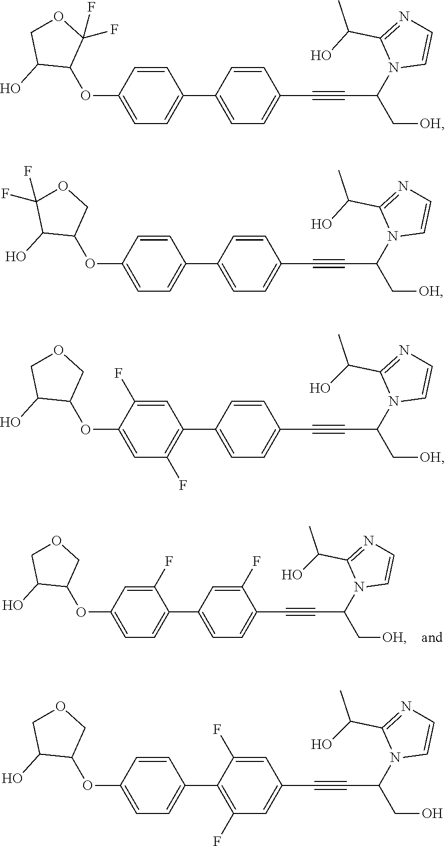 [SMILES: CC(O)c1nccn1C(C#Cc1ccc(-c2ccc(OC3C(O)COC3(F)F)cc2)cc1)CO ; CC(O)c1nccn1C(C#Cc1ccc(-c2ccc(OC3COC(F)(F)C3O)cc2)cc1)CO ; CC(O)c1nccn1C(C#Cc1ccc(-c2cc(F)c(OC3COCC3O)cc2F)cc1)CO ; CC(O)c1nccn1C(C#Cc1ccc(-c2ccc(OC3COCC3O)cc2F)cc1F)CO ; CC(O)c1nccn1C(C#Cc1cc(F)c(-c2ccc(OC3COCC3O)cc2)c(F)c1)CO]

In some embodiments, the compound is a compound of Table 1, or a pharmaceutically acceptable salt, solvate, or stereoisomer thereof. In some embodiments, the compound is a diastereomer of a compound of Table 1, or a pharmaceutically acceptable salt, solvate, or stereoisomer thereof.

TABLE 1

| Cmpd | | |
|---|---|---|
| No | Structure | IUPAC Name |
| 1 | 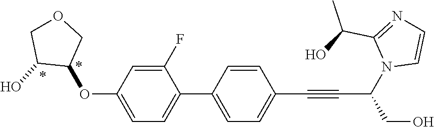 [SMILES: C[C@H](O)c1nccn1[C@@H](C#Cc1ccc(-c2ccc(O[C@@H]3COC[C@H]3O)cc2F)cc1)CO] | (rac-trans)-4-((2-fluoro-4′-((S)- 4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| | *racemic-trans | |
| 2** | 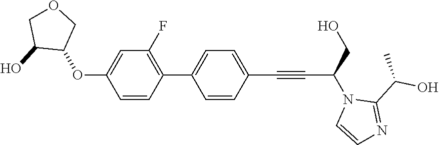 [SMILES: C[C@H](O)c1nccn1[C@@H](C#Cc1ccc(-c2ccc(O[C@H]3COC[C@@H]3O)cc2F)cc1)CO] | (3S,4S)-4-((2-fluoro-4′-((S)-4- hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| | (isomer 1 of Compound 1) | |
| 3** | 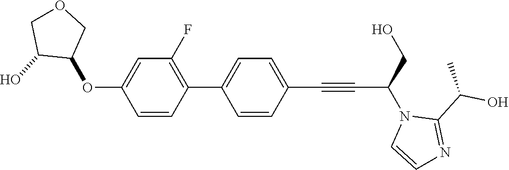 [SMILES: C[C@H](O)c1nccn1[C@@H](C#Cc1ccc(-c2ccc(O[C@@H]3COC[C@H]3O)cc2F)cc1)CO] | (3R,4R)-4-((2-fluoro-4′-((S)-4- hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| | (isomer 2 of Compound 1) | |
| 4 | 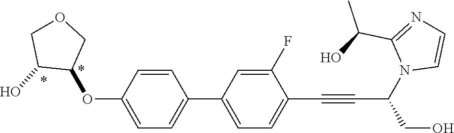 [SMILES: C[C@H](O)c1nccn1[C@@H](C#Cc1ccc(-c2ccc(O[C@@H]3COC[C@H]3O)cc2)cc1F)CO] | (rac-trans)-4-((3′-fluoro-4′-((S)- 4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| | *racemic-trans | |
| 5 | 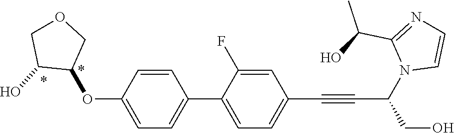 [SMILES: C[C@H](O)c1nccn1[C@@H](C#Cc1ccc(-c2ccc(O[C@@H]3COC[C@H]3O)cc2)c(F)c1)CO] | (rac-trans)-4-((2′-fluoro-4′-((S)- 4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| | *racemic-trans | |
| 6 | 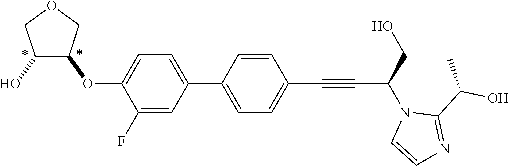 | (rac-trans)-4-((3-fluoro-4′-((S)- 4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| | *racemic-trans | |
| 7** | 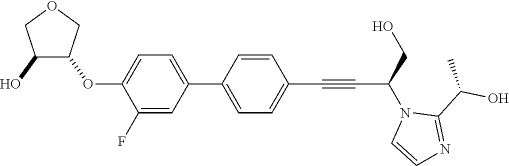 | (3S,4S)-4-((3-fluoro-4′-((S)-4- hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| | (isomer 1 of Compound 6) | |
| 8** | 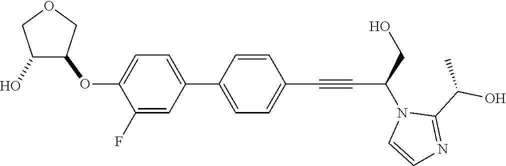 | (3R,4R)-4-((3-fluoro-4′-((S)-4- hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| | (isomer 2 of Compound 6) | |
| 9 | 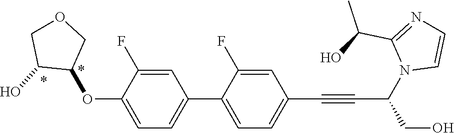 [SMILES: C[C@H](O)c1nccn1[C@@H](C#Cc1ccc(-c2ccc(O[C@@H]3COC[C@H]3O)c(F)c2)c(F)c1)CO] | (rac-trans)-4-((2′,3-difluoro-4′- ((S)-4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| | *racemic-trans | |
| 10** | 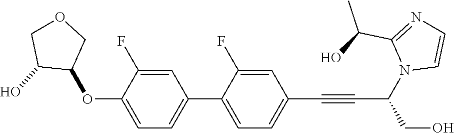 [SMILES: C[C@H](O)c1nccn1[C@@H](C#Cc1ccc(-c2ccc(O[C@@H]3COC[C@H]3O)c(F)c2)c(F)c1)CO] | (3R,4R)-4-((2′,3-difluoro-4′-((S)- 4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| | (isomer 1 of Compound 9) | |
| 11** | 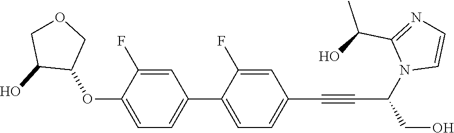 [SMILES: C[C@H](O)c1nccn1[C@@H](C#Cc1ccc(-c2ccc(O[C@H]3COC[C@@H]3O)c(F)c2)c(F)c1)CO] | (3S,4S)-4-((2′,3-difluoro-4′-((S)- 4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| | (isomer 2 of Compound 9) | |
| 12 | 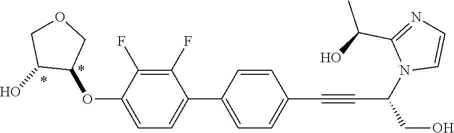 [SMILES: C[C@H](O)c1nccn1[C@@H](C#Cc1ccc(-c2ccc(O[C@@H]3COC[C@H]3O)c(F)c2F)cc1)CO] | (rac-trans)-4-((2,3-difluoro-4′- ((S)-4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| | *racemic-trans | |
| 13** | 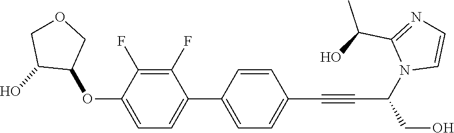 [SMILES: C[C@H](O)c1nccn1[C@@H](C#Cc1ccc(-c2ccc(O[C@@H]3COC[C@H]3O)c(F)c2F)cc1)CO] | (3R,4R)-4-((2,3-difluoro-4′-((S)- 4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| | (isomer 1 of Compound 12) | |
| 14** | 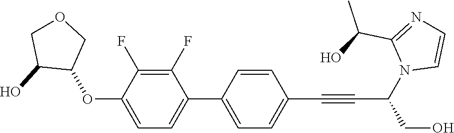 [SMILES: C[C@H](O)c1nccn1[C@@H](C#Cc1ccc(-c2ccc(O[C@H]3COC[C@@H]3O)c(F)c2F)cc1)CO] | (3S,4S)-4-((2,3-difluoro-4′-((S)- 4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| | (isomer 2 of Compound 12) | |
| 15 | 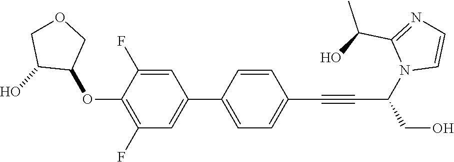 [SMILES: C[C@H](O)c1nccn1[C@@H](C#Cc1ccc(-c2cc(F)c(O[C@@H]3COC[C@H]3O)c(F)c2)cc1)CO] | (rac-trans)-4-((3,5-difluoro-4′- ((S)-4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| | *racemic-trans | |
| 16** | 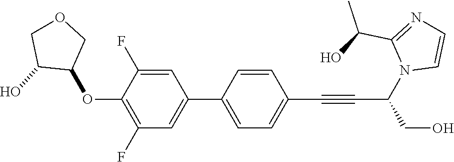 [SMILES: C[C@H](O)c1nccn1[C@@H](C#Cc1ccc(-c2cc(F)c(O[C@@H]3COC[C@H]3O)c(F)c2)cc1)CO] | (3R,4R)-4-((3,5-difluoro-4′-((S)- 4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| | (isomer 1 of Compound 15) | |
| 17** | 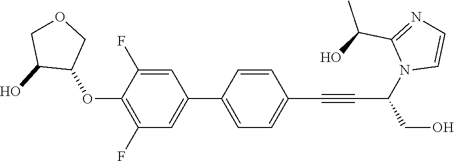 [SMILES: C[C@H](O)c1nccn1[C@@H](C#Cc1ccc(-c2cc(F)c(O[C@H]3COC[C@@H]3O)c(F)c2)cc1)CO] | (3S,4S)-4-((3,5-difluoro-4′-((S)- 4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| | (isomer 2 of Compound 15) | |
| 18 | 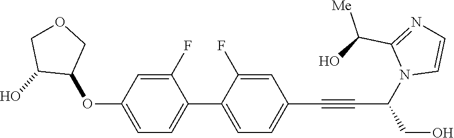 [SMILES: C[C@H](O)c1nccn1[C@@H](C#Cc1ccc(-c2ccc(O[C@@H]3COC[C@H]3O)cc2F)c(F)c1)CO] | (3R,4R)-4-((2,2′-difluoro-4′-((S)- 4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| 19 | 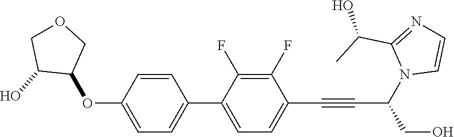 [SMILES: C[C@H](O)c1nccn1[C@@H](C#Cc1ccc(-c2ccc(O[C@@H]3COC[C@H]3O)cc2)c(F)c1F)CO] | (3R,4R)-4-((2′,3′-difluoro-4′- ((S)-4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| 20 | 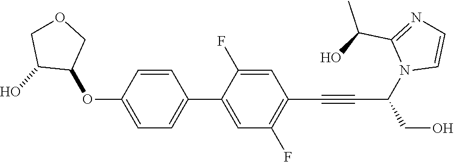 [SMILES: C[C@H](O)c1nccn1[C@@H](C#Cc1cc(F)c(-c2ccc(O[C@@H]3COC[C@H]3O)cc2)cc1F)CO] | (3R,4R)-4-((2′,5′-difluoro-4′- ((S)-4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| 21 | 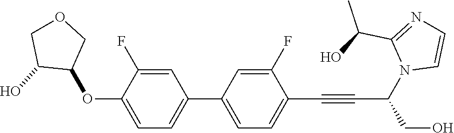 [SMILES: C[C@H](O)c1nccn1[C@@H](C#Cc1ccc(-c2ccc(O[C@@H]3COC[C@H]3O)c(F)c2)cc1F)CO] | (3R,4R)-4-((3,3′-difluoro-4′-((S)- 4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| 22 | 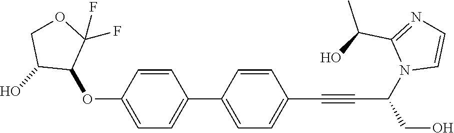 | (3R,4S)-5,5-difluoro-4-((4′-((S)- 4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| 23 | 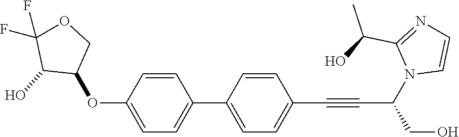 | (3S,4R)-2,2-difluoro-4-((4′-((S)- 4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| 24 | 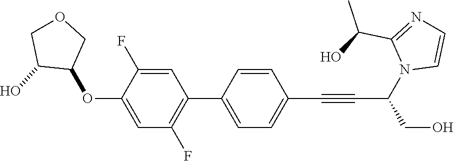 | (3R,4R)-4-((2,5-difluoro-4′-((S)- 4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| 25 | 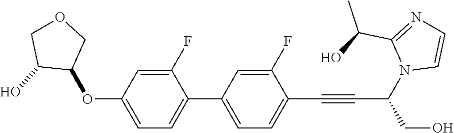 [SMILES: C[C@H](O)c1nccn1[C@@H](C#Cc1ccc(-c2ccc(O[C@@H]3COC[C@H]3O)cc2F)cc1F)CO] | (3R,4R)-4-((2,3′-difluoro-4′-((S)- 4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| 26 | 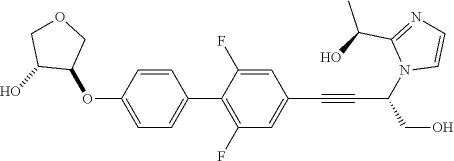 [SMILES: C[C@H](O)c1nccn1[C@@H](C#Cc1cc(F)c(-c2ccc(O[C@@H]3COC[C@H]3O)cc2)c(F)c1)CO] | (3R,4R)-4-((2′,6′-difluoro-4′- ((S)-4-hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| 44 | 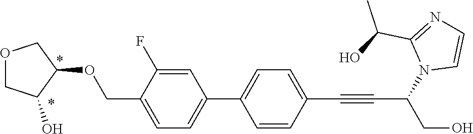 [SMILES: C[C@H](O)c1nccn1[C@@H](C#Cc1ccc(-c2ccc(CO[C@@H]3COC[C@H]3O)c(F)c2)cc1)CO] | (rac-trans)-4-((3-fluoro-4′-((S)- 4-hydroxy-3-(2-((S)-1- hydroxyethyl)-1H-imidazol-1- yl)but-1-yn-1-yl)-[1,1′- biphenyl]-4- yl)methoxy)tetrahydrofuran-3-ol |
| | *racemic-trans | |
| 45** | 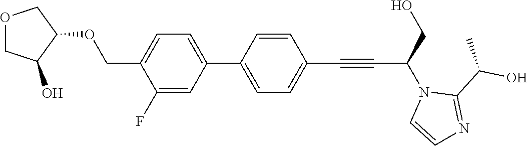 [SMILES: C[C@H](O)c1nccn1[C@@H](C#Cc1ccc(-c2ccc(CO[C@H]3COC[C@@H]3O)c(F)c2)cc1)CO] | (3S,4S)-4-((3-fluoro-4′-((S)-4- hydroxy-3-(2-((S)-1- hydroxyethyl)-1H-imidazol-1- yl)but-1-yn-1-yl)-[1,1′- biphenyl]-4- yl)methoxy)tetrahydrofuran-3-ol |
| | (isomer 1 of Compound 44) | |
| 46** | 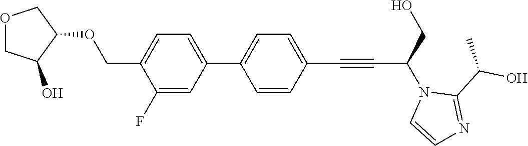 [SMILES: C[C@H](O)c1nccn1[C@@H](C#Cc1ccc(-c2ccc(CO[C@H]3COC[C@@H]3O)c(F)c2)cc1)CO] | (3S,4S)-4-((3-fluoro-4′-((S)-4- hydroxy-3-(2-((S)-1- hydroxyethyl)-1H-imidazol-1- yl)but-1-yn-1-yl)-[1,1′- biphenyl]-4- yl)methoxy)tetrahydrofuran-3-ol |
| | (isomer 2 of Compound 44) | |
| 47** | 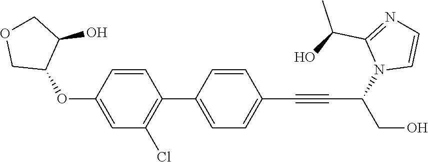 [SMILES: C[C@H](O)c1nccn1[C@@H](C#Cc1ccc(-c2ccc(O[C@@H]3COC[C@H]3O)cc2Cl)cc1)CO] | (3R,4R)-4-((2-chloro-4′-((S)-4- hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| 48** | 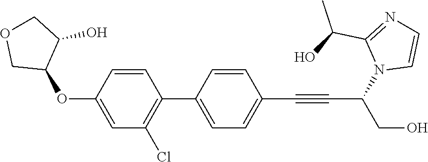 [SMILES: C[C@H](O)c1nccn1[C@@H](C#Cc1ccc(-c2ccc(O[C@H]3COC[C@@H]3O)cc2Cl)cc1)CO] | (3S,4S)-4-((2-chloro-4′-((S)-4- hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| 49** | 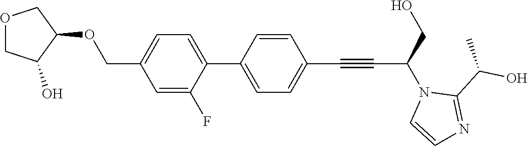 [SMILES: C[C@H](O)c1nccn1[C@@H](C#Cc1ccc(-c2ccc(CO[C@@H]3COC[C@H]3O)cc2F)cc1)CO] | (3R,4R)-4-((2-fluoro-4′-((S)-4- hydroxy-3-(2-((S)-1- hydroxyethyl)-1H-imidazol-1- yl)but-1-yn-1-yl)-[1,1′- biphenyl]-4- yl)methoxy)tetrahydrofuran-3-ol |
| 50** | 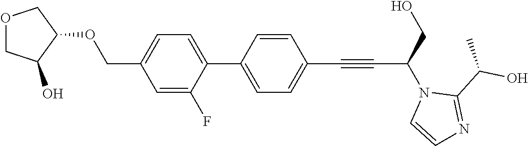 [SMILES: C[C@H](O)c1nccn1[C@@H](C#Cc1ccc(-c2ccc(CO[C@H]3COC[C@@H]3O)cc2F)cc1)CO] | (3S,4S)-4-((2-fluoro-4′-((S)-4- hydroxy-3-(2-((S)-1- hydroxyethyl)-1H-imidazol-1- yl)but-1-yn-1-yl)-[1,1′- biphenyl]-4- yl)methoxy)tetrahydrofuran-3-ol |
| 51 | 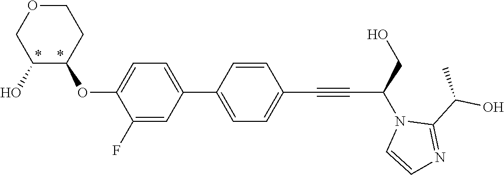 [SMILES: C[C@H](O)c1nccn1[C@@H](C#Cc1ccc(-c2ccc(O[C@@H]3CCOC[C@H]3O)c(F)c2)cc1)CO] | (rac-trans)-4-((3-fluoro-4′-((S)- 4-hydroxy-3-(2-((S)-1- hydroxyethyl)-1H-imidazol-1- yl)but-1-yn-1-yl)-[1,1′- biphenyl]-4-yl)oxy)tetrahydro- 2H-pyran-3-ol |
| 52** | 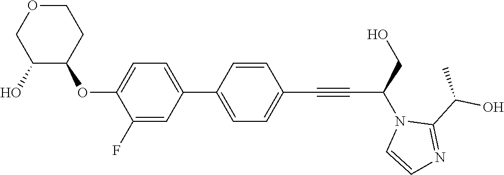 [SMILES: C[C@H](O)c1nccn1[C@@H](C#Cc1ccc(-c2ccc(O[C@@H]3CCOC[C@H]3O)c(F)c2)cc1)CO] | (3R,4R)-4-((3-fluoro-4′-((S)-4- hydroxy-3-(2-((S)-1- hydroxyethyl)-1H-imidazol-1- yl)but-1-yn-1-yl)-[1,1′- biphenyl]-4-yl)oxy)tetrahydro- 2H-pyran-3-ol |
| | (isomer 1 of compound 45) | |
| 53** | 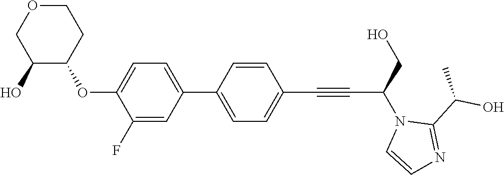 [SMILES: C[C@H](O)c1nccn1[C@@H](C#Cc1ccc(-c2ccc(O[C@H]3CCOC[C@@H]3O)c(F)c2)cc1)CO] | (3S,4S)-4-((3-fluoro-4′-((S)-4- hydroxy-3-(2-((S)-1- hydroxyethyl)-1H-imidazol-1- yl)but-1-yn-1-yl)-[1,1′- biphenyl]-4-yl)oxy)tetrahydro- 2H-pyran-3-ol |
| | (isomer 2 of compound 45) | |
| 54 | 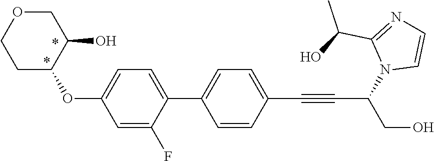 [SMILES: C[C@H](O)c1nccn1[C@@H](C#Cc1ccc(-c2ccc(O[C@@H]3CCOC[C@H]3O)cc2F)cc1)CO] | (rac-trans)-4-((2-fluoro-4′-((S)- 4-hydroxy-3-(2-((S)-1- hydroxyethyl)-1H-imidazol-1- yl)but-1-yn-1-yl)-[1,1′- biphenyl]-4-yl)oxy)tetrahydro- 2H-pyran-3-ol |
| | *racemic-trans | |
| 55** | 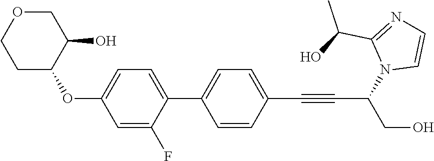 [SMILES: C[C@H](O)c1nccn1[C@@H](C#Cc1ccc(-c2ccc(O[C@@H]3CCOC[C@H]3O)cc2F)cc1)CO] | (3R,4R)-4-((2-fluoro-4′-((S)-4- hydroxy-3-(2-((S)-1- hydroxyethyl)-1H-imidazol-1- yl)but-1-yn-1-yl)-[1,1′- biphenyl]-4-yl)oxy)tetrahydro- 2H-pyran-3-ol |
| | (isomer 1 of compound 48) | |
| 56** | 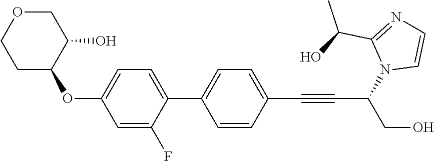 [SMILES: C[C@H](O)c1nccn1[C@@H](C#Cc1ccc(-c2ccc(O[C@H]3CCOC[C@@H]3O)cc2F)cc1)CO] | (3S,4S)-4-((2-fluoro-4′-((S)-4- hydroxy-3-(2-((S)-1- hydroxyethyl)-1H-imidazol-1- yl)but-1-yn-1-yl)-[1,1′- biphenyl]-4-yl)oxy)tetrahydro- 2H-pyran-3-ol |
| | (isomer 2 of compound 48) | |
| 57** | 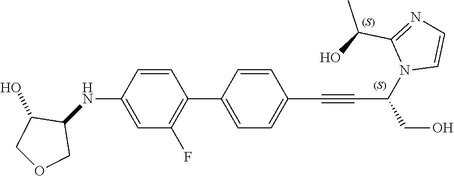 | (3R,4S)-4-((2-fluoro-4′-((S)-4- hydroxy-3-(2-((S)-1- hydroxyethyl)-1H-imidazol-1- yl)but-1-yn-1-yl)-[1,1′- biphenyl]-4- yl)amino)tetrahydrofuran-3-ol |
| 58** | 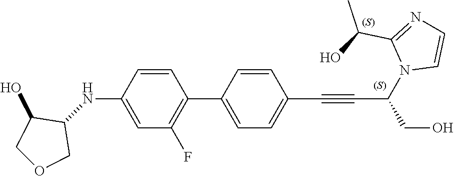 | (3S,4R)-4-((2-fluoro-4′-((S)-4- hydroxy-3-(2-((S)-1- hydroxyethyl)-1H-imidazol-1- yl)but-1-yn-1-yl)-[1,1′- biphenyl]-4- yl)amino)tetrahydrofuran-3-ol |
| 59** | 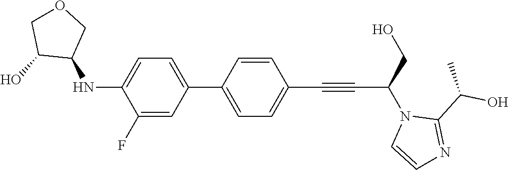 [SMILES: C[C@H](O)c1nccn1[C@@H](C#Cc1ccc(-c2ccc(N[C@@H]3COC[C@H]3O)c(F)c2)cc1)CO] | (3S,4R)-4-((3-fluoro-4′-((S)-4- hydroxy-3-(2-((S)-1- hydroxyethyl)-1H-imidazol-1- yl)but-1-yn-1-yl)-[1,1′- biphenyl]-4- yl)amino)tetrahydrofuran-3-ol |
| 60** | 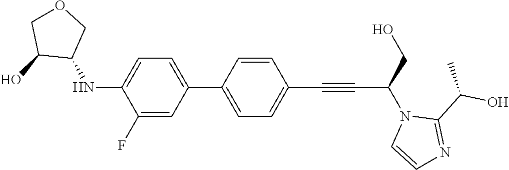 [SMILES: C[C@H](O)c1nccn1[C@@H](C#Cc1ccc(-c2ccc(N[C@H]3COC[C@@H]3O)c(F)c2)cc1)CO] | (3R,4S)-4-((3-fluoro-4′-((S)-4- hydroxy-3-(2-((S)-1- hydroxyethyl)-1H-imidazol-1- yl)but-1-yn-1-yl)-[1,1′- biphenyl]-4- yl)amino)tetrahydrofuran-3-ol |
| 61** | 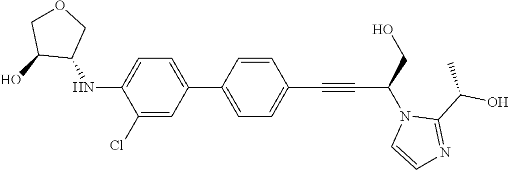 [SMILES: C[C@H](O)c1nccn1[C@@H](C#Cc1ccc(-c2ccc(N[C@H]3COC[C@@H]3O)c(Cl)c2)cc1)CO] | (3S,4S)-4-((3-chloro-4′-((S)-4- hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| 62** | 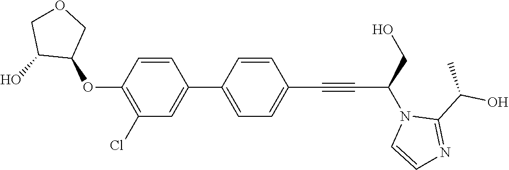 [SMILES: C[C@H](O)c1nccn1[C@@H](C#Cc1ccc(-c2ccc(O[C@@H]3COC[C@H]3O)c(Cl)c2)cc1)CO] | (3R,4R)-4-((3-chloro-4′-((S)-4- hydroxy-3-(2-((S)-1- hydroxyethyl)-1H-imidazol-1- yl)but-1-yn-1-yl)-[1,1′- biphenyl]-4- yl)oxy)tetrahydrofuran-3-ol |
| 63 | 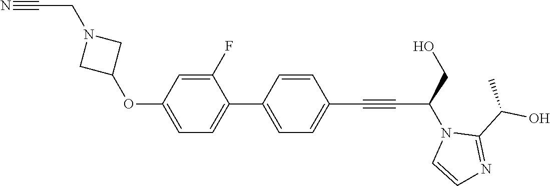 [SMILES: C[C@H](O)c1nccn1[C@@H](C#Cc1ccc(-c2ccc(OC3CN(CC#N)C3)cc2F)cc1)CO] | 2-(3-((2-fluoro-4′-((S)-4- hydroxy-3-(2-((S)-1- hydroxyethyl)-1H-imidazol-1- yl)but-1-yn-1-yl)-[1,1′- biphenyl]-4-yl)oxy)azetidin-1- yl)acetonitrile |
| 64 | 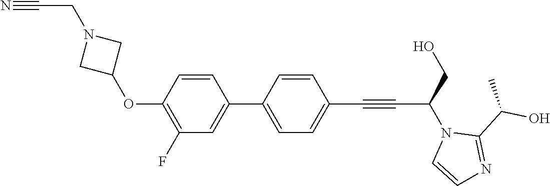 [SMILES: C[C@H](O)c1nccn1[C@@H](C#Cc1ccc(-c2ccc(OC3CN(CC#N)C3)c(F)c2)cc1)CO] | 2-(3-((3-fluoro-4′-((S)-4- hydroxy-3-(2-((S)-1- hydroxyethyl)-1H-imidazol-1- yl)but-1-yn-1-yl)-[1,1′- biphenyl]-4-yl)oxy)azetidin-1- yl)acetonitrile |

**stereochemistry arbitrarily assigned. After chiral separation, single stereoisomers are isolated but the absolute configuration of the stereochemical center is unknown.

In another aspect, the present disclosure provides compound having the structure of Formula (VI):

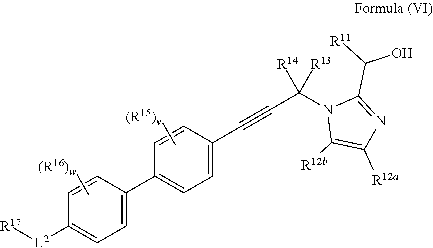

or a pharmaceutically acceptable salt, solvate, or stereoisomer thereof, wherein:
   R¹¹ is C₁-C₄ alkyl;
   R^{12a} and R^{12b} are each independently hydrogen, halogen, or C₁-C₄ alkyl;
   R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂;
   R¹⁴ is hydrogen or C₁-C₄ alkyl;
   each R¹⁵ and R¹⁶ is independently halogen, or C₁-C₄ alkyl;
   R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 groups independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; wherein:
      each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃;
      or two R¹⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo;
   L² is a bond, —(C₁-C₆ alkylene)-, —X³—, or —X⁴—(C₁-C₆ alkylene)-, wherein:
      X³ and X⁴ are each independently selected from: —O—, —N(R¹⁹)—, —S—, —S(═O)—, —S(═O)₂—, and —S(═O)(═NR¹⁹)—, wherein:
         R¹⁹ is hydrogen or C₁-C₆ alkyl;
   v is 0, 1, or 2; and
   w is 0, 1, or 2.

In some embodiments, when L² is a bond, —(C₁-C₆ alkylene)-, —O—, or —O—(C₁-C₆ alkylene)- then at least one of the following:
(i) R¹³ is —(C₃-C₆ cycloalkylene)-OH or —(C₃-C₆ cycloalkylene)-NH₂;
(ii) R¹⁴ is C₁-C₄ alkyl;
(iii) R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂; or
(iv) L² is —O—(C₁-C₆ alkylene)- and R¹⁷ is an unsubstituted or substituted 5-membered heterocycloalkyl.

In some embodiments, when L² is a bond, —(C₁-C₆ alkylene)-, —O—, or —O—(C₁-C₆ alkylene)-, then R¹³ is —(C₃-C₆ cycloalkylene)-OH or —(C₃-C₆ cycloalkylene)-NH₂.

In some embodiments, when L² is a bond, —(C₁-C₆ alkylene)-, —O—, or —O—(C₁-C₆ alkylene)-, then R¹⁴ is C₁-C₄ alkyl.

In some embodiments, when L² is a bond, —(C₁-C₆ alkylene)-, —O—, or —O—(C₁-C₆ alkylene)-, then R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂.

In some embodiments, when L² is a bond, —(C₁-C₆ alkylene)-, —O—, or —O—(C₁-C₆ alkylene)-, then L² is —O—(C₁-C₆ alkylene)- and R¹⁷ is an unsubstituted or substituted 5-membered heterocycloalkyl.

In some embodiments of a compound of Formula (VI), R¹¹ is —CH₃; R^{12a} and R^{12b} are each hydrogen; R¹³ is hydrogen, —(C₁-C₄ alkylene)-OH, or —(C₁-C₄ alkylene)-NH₂; R¹⁴ is hydrogen; each R¹⁵ and R¹⁶ is independently —F; R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 groups independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; L² is a bond, —(C₁-C₆ alkylene)-, —X³—, or —X⁴—(C₁-C₆ alkylene)-, wherein: X³ and X⁴ are each independently selected from: —O—, —N(R¹⁹)—, —S—, —S(═O)—, —S(═O)₂—, and —S(═O)(═NR¹⁹)—, wherein: R¹⁹ is hydrogen or C₁-C₆ alkyl; provided that when L² is a bond, —(C₁-C₆ alkylene)-, —O—, or —O—(C₁-C₆ alkylene)- then at least one of the following: (i) R¹³ is —(C₃-C₆ cycloalkylene)-OH or —(C₃-C₆ cycloalkylene)-NH₂; (ii) R¹⁴ is C₁-C₄ alkyl, (iii) R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂; or (iv) L² is —O—(C₁-C₆ alkylene)- and R¹⁷ is an unsubstituted or substituted 5-membered heterocycloalkyl; v is 0, 1, or 2; and w is 0, 1, or 2.

In some embodiments of a compound of Formula (VI), R¹¹ is C₁-C₄ alkyl; R^{12a} and R^{12b} are each independently hydrogen, halogen, or C₁-C₄ alkyl; R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂; R¹⁴ is hydrogen or C₁-C₄ alkyl; each R¹⁵ and R¹⁶ is independently halogen, or C₁-C₄ alkyl; R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 groups independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo; L² is —X³—, or —X⁴—(C₁-C₆ alkylene)-, wherein: X³ and X⁴ are each independently selected from: —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; and w is 0, 1, or 2.

In some embodiments of a compound of Formula (VI), R¹¹ is —CH₃; R^{12a} and R^{12b} are each hydrogen; R¹³ is hydrogen, —(C₁-C₄ alkylene)-OH, —(C₁-C₄ alkylene)-NH₂, —(C₃-C₆ cycloalkylene)-OH or —(C₃-C₆ cycloalkylene)-NH₂; R¹⁴ is hydrogen; each R¹⁵ and R¹⁶ is independently —F; R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 groups independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; L² is a bond, —(C₁-C₆ alkylene)-, —O—, or —O—(C₁-C₆ alkylene)- then at least one of the following: (i) R¹³ is —(C₃-C₆ cycloalkylene)-OH or —(C₃-C₆ cycloalkylene)-NH₂; (ii) R¹⁴ is C₁-C₄ alkyl; (iii) R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂; v is 0, 1, or 2; and w is 0, 1, or 2.

In another aspect, the present disclosure provides compound having the structure of Formula (VIa) or (VIb):

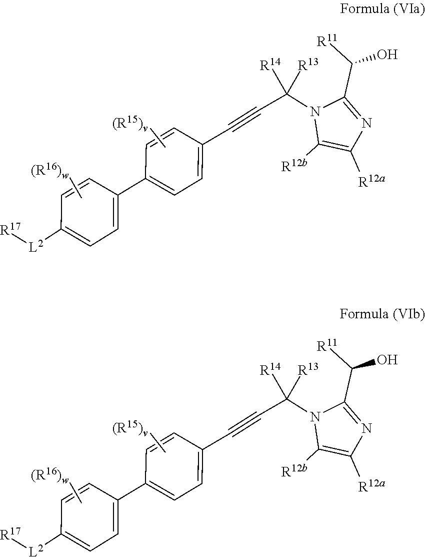

or a pharmaceutically acceptable salt, solvate, or stereoisomer thereof.

In some embodiments of a compound of Formula (VI), (VIa), or (VIb), R¹¹ is C₁-C₄ alkyl; R^{12a} and R^{12b} are each independently hydrogen, halogen, or C₁-C₄ alkyl; R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂; R¹⁴ is hydrogen or C₁-C₄ alkyl; each R¹⁵ and R¹⁶ is independently halogen, or C₁-C₄ alkyl; R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 groups independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo; L² is a bond, —(C₁-C₆ alkylene)-, —X³—, or —X⁴—(C₁-C₆ alkylene)-, wherein: X³ and X⁴ are each independently selected from: —O—, —N(R¹⁹)—, —S—, —S(═O)—, —S(═O)₂—, and —S(═O)(═NR¹⁹)—, wherein: R¹⁹ is hydrogen or C₁-C₆ alkyl; provided that when L² is a bond, —(C₁-C₆ alkylene)-, —O—, or —O—(C₁-C₆ alkylene)- then at least one of the following: (i) R¹³ is —(C₃-C₆ cycloalkylene)-OH or —(C₃-C₆ cycloalkylene)-NH₂; (ii) R¹⁴ is C₁-C₄ alkyl, (iii) R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂; or (iv) L² is —O—(C₁-C₆ alkylene)- and R¹⁷ is an unsubstituted or substituted 5-membered heterocycloalkyl; v is 0, 1, or 2; and w is 0, 1, or 2.

In some embodiments of a compound of Formula (VIa) or (VIb), R¹¹ is —CH₃; R^{12a} and R^{12b} are each hydrogen; R¹³ is hydrogen, —(C₁-C₄ alkylene)-OH, or —(C₁-C₄ alkylene)-NH₂; R¹⁴ is hydrogen; each R¹⁵ and R¹⁶ is independently —F; R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 groups independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; L² is a bond, —(C₁-C₆ alkylene)-, —X³—, or —X⁴—(C₁-C₆ alkylene)-, wherein: X³ and X⁴ are each independently selected from: —O—, —N(R¹⁹)—, —S—, —S(═O)—, —S(═O)₂—, and —S(═O)(═NR¹⁹)—, wherein: R¹⁹ is hydrogen or C₁-C₆ alkyl; provided that when L² is a bond, —(C₁-C₆ alkylene)-, —O—, or —O—(C₁-C₆ alkylene)- then at least one of the following: (i) R¹³ is —(C₃-C₆ cycloalkylene)-OH or —(C₃-C₆ cycloalkylene)-NH₂; (ii) R¹⁴ is C₁-C₄ alkyl, (iii) R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂; or (iv) L² is —O—(C₁-C₆ alkylene)- and R¹⁷ is an unsubstituted or substituted 5-membered heterocycloalkyl; v is 0, 1, or 2; and w is 0, 1, or 2.

In some embodiments of a compound of Formula (VI), (VIa), or (VIb), R¹¹ is C₁-C₄ alkyl; R^{12a} and R^{12b} are each independently hydrogen, halogen, or C₁-C₄ alkyl; R¹³ is —(C₃-C₆ cycloalkylene)-OH, or —(C₃-C₆ cycloalkylene)-NH₂; R¹⁴ is hydrogen or C₁-C₄ alkyl; each R¹⁵ and R¹⁶ is independently halogen, or C₁-C₄ alkyl; R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 groups independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo; L² is a bond, —(C₁-C₆ alkylene)-, —X³—, or —X⁴—(C₁-C₆ alkylene)-, wherein: X³ and X⁴ are each independently selected from: —O—, —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; and w is 0, 1, or 2.

In some embodiments of a compound of Formula (VI), (VIa), or (VIb), R¹¹ is C₁-C₄ alkyl; R^{12a} and R^{12b} are each independently hydrogen, halogen, or C₁-C₄ alkyl; R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂; R¹⁴ is C₁-C₄ alkyl; each R¹⁵ and R¹⁶ is independently halogen, or C₁-C₄ alkyl; R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 groups independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo; L² is a bond, —(C₁-C₆ alkylene)-, —X³—, or —X⁴—(C₁-C₆ alkylene)-, wherein: X³ and X⁴ are each independently selected from: —O—, —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; and w is 0, 1, or 2.

In some embodiments of a compound of Formula (VI), (VIa), or (VIb), R¹¹ is C₁-C₄ alkyl; R^{12a} and R^{12b} are each independently hydrogen, halogen, or C₁-C₄ alkyl; R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂; R¹⁴ is hydrogen or C₁-C₄ alkyl; each R¹⁵ and R¹⁶ is independently halogen, or C₁-C₄ alkyl; R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂; each of which is unsubstituted or substituted by 1, 2, or 3 groups independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo; L² is a bond, —(C₁-C₆ alkylene)-, —X³—, or —X⁴—(C₁-C₆ alkylene)-, wherein: X³ and X⁴ are each independently selected from: —O—, —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; and w is 0, 1, or 2.

In some embodiments of a compound of Formula (VI), (VIa), or (VIb), R¹¹ is C₁-C₄ alkyl; R^{12a} and R^{12b} are each independently hydrogen, halogen, or C₁-C₄ alkyl; R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂; R¹⁴ is hydrogen or C₁-C₄ alkyl; each R¹⁵ and R¹⁶ is independently halogen, or C₁-C₄ alkyl; R¹⁷ is a 5-membered heterocycloalkyl unsubstituted or substituted by 1, 2, or 3 groups independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo; L² is —O—(C₁-C₆ alkylene)-; v is 0, 1, or 2; and w is 0, 1, or 2.

In some embodiments of a compound of Formula (VI), (VIa), or (VIb), R¹¹ is C₁-C₄ alkyl; R^{12a} and R^{12b} are each independently hydrogen, halogen, or C₁-C₄ alkyl; R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂; R¹⁴ is hydrogen or C₁-C₄ alkyl; each R¹⁵ and R¹⁶ is independently halogen, or C₁-C₄ alkyl; R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 groups independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo; L² is —X³—, or —X⁴—(C₁-C₆ alkylene)-, wherein: X³ and X⁴ are each independently selected from: —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; and w is 0, 1, or 2.

In some embodiments of a compound of Formula (I), (Ia), or (Ib), R¹¹ is —CH₃; R^{12a} and R^{12b} are each hydrogen; R¹³ is hydrogen, —(C₁-C₄ alkylene)-OH, or —(C₁-C₄ alkylene)-NH₂; R¹⁴ is hydrogen; each R¹⁵ and R¹⁶ is —F; R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R²⁰ groups, wherein; each R²⁰ is independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; L² is a bond, —(C₁-C₆ alkylene)-, —X³—, or —X⁴—(C₁-C₆ alkylene)-, wherein: X³ and X⁴ are each independently selected from: —O—, —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; and w is 0, 1, or 2; provided that when L² is a bond, —(C₁-C₆ alkylene)-, —O—, or —O—(C₁-C₆ alkylene)- then R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂.

In some embodiments of a compound of Formula (I), (Ia), or (Ib), R¹¹ is —CH₃; R^{12a} and R^{12b} are each hydrogen; R¹³ is hydrogen, —(C₁-C₄ alkylene)-OH, or —(C₁-C₄ alkylene)-NH₂; R¹⁴ is hydrogen; each R¹⁵ and R¹⁶ is —F; R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R²⁰ groups, wherein; each R²⁰ is independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; L² is a —X³—, or —X⁴—(C₁-C₆ alkylene)-, wherein: X³ and X⁴ are each independently selected from: —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; and w is 0, 1, or 2.

In some embodiments of a compound of Formula (I), (Ia), or (Ib), R¹¹ is —CH₃; R^{12a} and R^{12b} are each hydrogen; R¹³ is hydrogen or —CH₂OH; R¹⁴ is hydrogen; each R¹⁵ and R¹⁶ is —F; R¹⁷ is 4- to 8-membered heterocycloalkyl, where the 4- to 8-membered heterocycloalkyl is unsubstituted or substituted by 1, 2, or 3 R²⁰ groups, wherein: each R²⁰ is independently selected from: halogen, —OH, —OMe, —N(R¹⁸)₂, —NHSO₂R¹⁸, and —CH₂CN; and each R¹⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl; L² is a bond, —(C₁-C₆ alkylene)-, —X³—, or —X⁴—(C₁-C₆ alkylene)-, wherein: X³ and X⁴ are each independently selected from: —O—, —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; and w is 0, 1, or 2; provided that when L² is a bond, —(C₁-C₆ alkylene)-, —O—, or —O—(C₁-C₆ alkylene)- then R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂.

In some embodiments of a compound of Formula (I), (Ia), or (Ib), R¹¹ is —CH₃; R^{12a} and R^{12b} are each hydrogen; R¹³ is hydrogen or —CH₂OH; R¹⁴ is hydrogen; each R¹⁵ and R¹⁶ is —F; R¹⁷ is 4- to 8-membered heterocycloalkyl, where the 4- to 8-membered heterocycloalkyl is unsubstituted or substituted by 1, 2, or 3 R²⁰ groups, wherein: each R²⁰ is independently selected from: halogen, —OH, —OMe, —N(R¹⁸)₂, —NHSO₂R¹⁸, and —CH₂CN; and each R¹⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl; L² is a —X³—, or —X⁴—(C₁-C₆ alkylene)-, wherein: X³ and X⁴ are each independently selected from: —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; and w is 0, 1, or 2;

In some embodiments of a compound of Formula (I), (Ia), or (Ib), R¹¹ is —CH₃; R^{12a} and R^{12b} are each hydrogen; R¹³ is hydrogen or —CH₂OH; R¹⁴ is hydrogen; each R¹⁵ and R¹⁶ is —F; R¹⁷ is 4- to 6-membered heterocycloalkyl, where the 4- to 6-membered heterocycloalkyl is unsubstituted or substituted by 1, 2, or 3 R²⁰ groups, wherein: each R²⁰ is independently selected from: —F, —OH, —OCH₃, and —NH; L² is a bond, —(C₁-C₆ alkylene)-, —X³—, or —X⁴—(C₁-C₆ alkylene)-, wherein: X³ and X⁴ are each independently selected from: —O—, —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; and w is 0, 1, or 2; provided that when L² is a bond, —(C₁-C₆ alkylene)-, —O—, or —O—(C₁-C₆ alkylene)- then R¹⁷ is an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂.

In some embodiments of a compound of Formula (I), (Ia), or (Ib), R¹¹ is —CH₃; R^{12a} and R^{12b} are each hydrogen; R¹³ is hydrogen or —CH₂OH; R¹⁴ is hydrogen; each R¹⁵ and R¹⁶ is —F; R¹⁷ is 4- to 6-membered heterocycloalkyl, where the 4- to 6-membered heterocycloalkyl is unsubstituted or substituted by 1, 2, or 3 R²⁰ groups, wherein: each R²⁰ is independently selected from: —F, —OH, —OCH₃, and —NH₂; L² is a —X³—, or —X⁴—(C₁-C₆ alkylene)-, wherein: X³ and X⁴ are each independently selected from: —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; and w is 0, 1, or 2.

In some embodiments, the compound is a compound of Formula (VIa), or a pharmaceutically acceptable salt, solvate, or stereoisomer thereof. In some embodiments, the compound is a compound of Formula (VIb), or a pharmaceutically acceptable salt, solvate, or stereoisomer thereof.

For any and all of the embodiments, substituents are selected from among a subset of the listed alternatives. For example, in some embodiments of a compound of Formula (VI), (VIa), or (VIb), R¹¹ is unsubstituted C₁-C₄ alkyl. In some embodiments, R¹¹ is C₁-C₂ alkyl. In some embodiments, R¹¹ is —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃. In some embodiments, R¹¹ is —CH₃ or —CH₂CH₃. In some embodiments, R¹¹ is —CH₃.

In some embodiments of a compound, salt, solvate, or stereoisomer of Formula (VI), (VIa), or (VIb), R^{12a} and R^{12b} are each independently R^{12a} and R^{12b} are each independently hydrogen, halogen, or unsubstituted C₁-C₄ alkyl. In some embodiments, R^{12a} and R^{12b} are each independently hydrogen, —F, —Cl, —Br, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, R^{12a} and R^{12b} are each independently hydrogen, —F, —Cl, —CH₃, —CH₂CH₃, or —CH(CH₃)₂.

In some embodiments of a compound, salt, solvate, or stereoisomer of Formula (VI), (VIa), or (VIb), R^{12a} is hydrogen. In some embodiments, R^{12b} is hydrogen. In some embodiments, R^{12a} and R^{12b} are each hydrogen.

In some embodiments of a compound, salt, solvate, or stereoisomer of Formula (VI), (VIa), or (VIb), R¹¹ is —CH₃; R^{12a} is hydrogen; and R^{12b} is hydrogen.

In some embodiments of a compound, salt, solvate, or stereoisomer of Formula (VI), (VIa), or (VIb), R¹⁴ is hydrogen or unsubstituted C₁-C₄ alkyl. In some embodiments, R¹⁴ is hydrogen or C₁-C₂ alkyl. In some embodiments, R¹⁴ is hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃. In some embodiments, R¹⁴ is hydrogen, —CH₃ or —CH₂CH₃. In some embodiments, R¹⁴ is hydrogen or —CH₃. In some embodiments, R¹⁴ is hydrogen. In some embodiments, R¹⁴ is —CH₃.

In some embodiments, the compound is a compound of Formula (VII), or a pharmaceutically acceptable salt, solvate, or stereoisomer thereof. In some embodiments, the compound is a compound of Formula (VIIa), or a pharmaceutically acceptable salt, solvate, or stereoisomer thereof. In some embodiments, the compound is a compound of Formula (VIIb), or a pharmaceutically acceptable salt, solvate, or stereoisomer thereof.

In some embodiments, the compound of Formula (VI) or (VII) is a compound of Formula (VII):

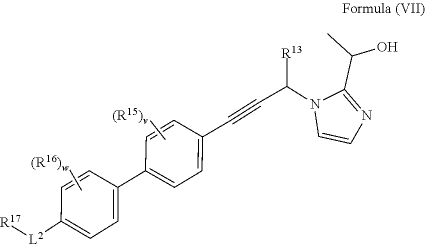

or a pharmaceutically acceptable salt, solvate, or stereoisomer thereof.

In some embodiments of a compound of Formula (VII), R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂; each R¹⁵ and R¹⁶ is independently halogen, or C₁-C₄ alkyl; R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 groups independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo; L² is a bond, —(C₁-C₆ alkylene)-, —X³—, or —X⁴—(C₁-C₆ alkylene)-, wherein: X³ and X⁴ are each independently selected from: —O—, —N(R¹⁹)—, —S—, —S(═O)—, —S(═O)₂—, and —S(═O)(═NR¹⁹)—, wherein: R¹⁹ is hydrogen or C₁-C₆ alkyl; provided that when L² is a bond, —(C₁-C₆ alkylene)-, —O—, or —O—(C₁-C₆ alkylene)- then at least one of the following: (i) R¹³ is —(C₃-C₆ cycloalkylene)-OH or —(C₃-C₆ cycloalkylene)-NH₂; (iii) R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂; or (iv) L² is —O—(C₁-C₆ alkylene)- and R¹⁷ is an unsubstituted or substituted 5-membered heterocycloalkyl; v is 0, 1, or 2; and w is 0, 1, or 2.

In some embodiments of a compound of Formula (VII), R¹³ is hydrogen, —(C₁-C₄ alkylene)-OH, or —(C₁-C₄ alkylene)-NH₂; R¹⁴ is hydrogen; each R¹⁵ and R¹⁶ is independently —F; R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 groups independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; L² is a bond, —(C₁-C₆ alkylene)-, —X³—, or —X⁴—(C₁-C₆ alkylene)-, wherein: X³ and X⁴ are each independently selected from: —O—, —N(R¹⁹)—, —S—, —S(═O)—, —S(═O)₂—, and —S(═O)(═NR¹⁹)—, wherein: R¹⁹ is hydrogen or C₁-C₆ alkyl; provided that when L² is a bond, —(C₁-C₆ alkylene)-, —O—, or —O—(C₁-C₆ alkylene)- then R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂; v is 0, 1, or 2; and w is 0, 1, or 2.

In some embodiments of a compound of Formula (VII), R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂; R¹⁵ and R¹⁶ is independently halogen, or C₁-C₄ alkyl; R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 groups independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo; L² is —X³—, or —X⁴—(C₁-C₆ alkylene)-, wherein: X³ and X⁴ are each independently selected from: —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; and w is 0, 1, or 2.

In some embodiments of a compound of Formula (VI), R¹³ is hydrogen, —(C₁-C₄ alkylene)-OH, —(C₁-C₄ alkylene)-NH₂, —(C₃-C₆ cycloalkylene)-OH or —(C₃-C₆ cycloalkylene)-NH₂; each R¹⁵ and R¹⁶ is independently —F; R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 groups independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; L² is a bond, —(C₁-C₆ alkylene)-, —O—, or —O—(C₁-C₆ alkylene)- then at least one of the following: (i) R¹³ is —(C₃-C₆ cycloalkylene)-OH or —(C₃-C₆ cycloalkylene)-NH₂; (ii) R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂; v is 0, 1, or 2; and w is 0, 1, or 2.

In some embodiments, the compound of Formula (VI) or (VII) is a compound of Formula (VIIa) or Formula (VIIb):

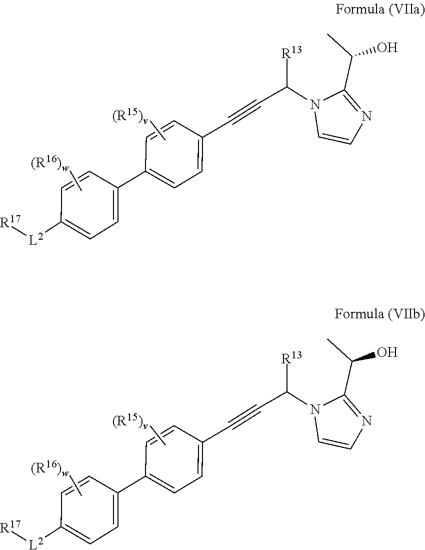

or a pharmaceutically acceptable salt, solvate, or stereoisomer thereof.

In some embodiments of a compound of Formula (VIIa) or (VIIb), R¹³ is hydrogen, —(C₁-C₄ alkylene)-OH, or —(C₁-C₄ alkylene)-NH₂; each R¹⁵ and R¹⁶ is independently —F; R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 groups independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; L² is a bond, —(C₁-C₆ alkylene)-, —X³—, or —X⁴—(C₁-C₆ alkylene)-, wherein: X³ and X⁴ are each independently selected from: —O—, —N(R¹⁹)—, —S—, —S(═O)—, —S(═O)₂—, and —S(═O)(═NR¹⁹)—, wherein: R¹⁹ is hydrogen or C₁-C₆ alkyl; provided that when L² is a bond, —(C₁-C₆ alkylene)-, —O—, or —O—(C₁-C₆ alkylene)- then R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂; v is 0, 1, or 2; and w is 0, 1, or 2.

In some embodiments of a compound of Formula (VII), (VIIa), or (VIIb), R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂; each R¹⁵ and R¹⁶ is independently halogen, or C₁-C₄ alkyl; R¹⁷ is a 5-membered heterocycloalkyl unsubstituted or substituted by 1, 2, or 3 groups independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo; L² is —O—(C₁-C₆ alkylene)-; v is 0, 1, or 2; and w is 0, 1, or 2.

In some embodiments of a compound of Formula (VII), (VIIa), or (VIIb), R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂; each R¹⁵ and R¹⁶ is independently halogen, or C₁-C₄ alkyl; R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 groups independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo; L² is —X³—, or —X⁴—(C₁-C₆ alkylene)-, wherein: X³ and X⁴ are each independently selected from: —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; and w is 0, 1, or 2.

In some embodiments, the compound is a compound of Formula (VIIa), or a pharmaceutically acceptable salt, solvate, or stereoisomer thereof. In some embodiments, the compound is a compound of Formula (VIIb), or a pharmaceutically acceptable salt, solvate, or stereoisomer thereof.

In some embodiments, the compound of Formula (VI) or (VII) is a compound of Formula (VIIIa), Formula (VIIIb), Formula (VIIIc), or Formula (VIIId):

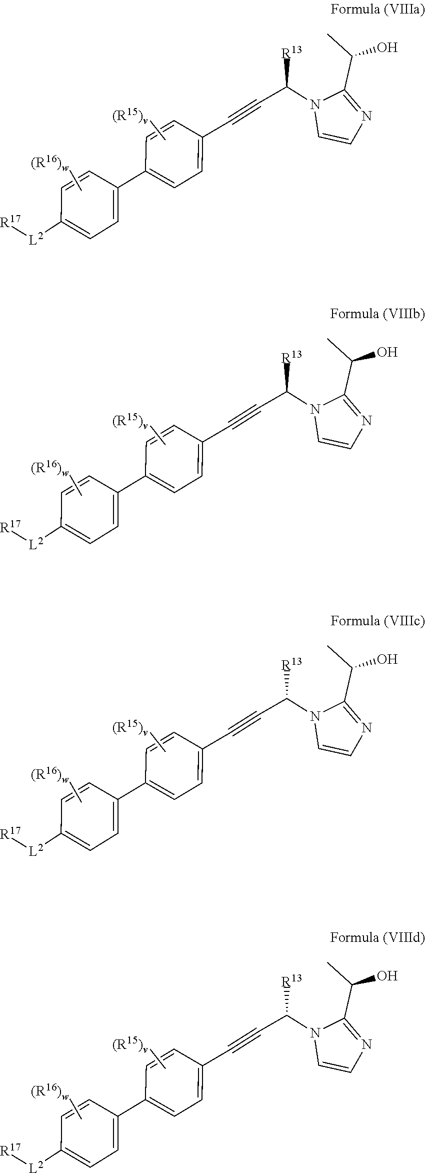

or a pharmaceutically acceptable salt, solvate, or stereoisomer thereof.

In some embodiments of a compound of Formula (VII), (VIIa), (VIIb), (VIIIa), (VIIIb), (VIIIc), or (VIIId), R¹³ is hydrogen, —(C₁-C₄ alkylene)-OH, or —(C₁-C₄ alkylene)-NH₂; each R¹⁵ and R¹⁶ is —F; R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R²⁰ groups, wherein; each R²⁰ is independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; L² is a bond, —(C₁-C₆ alkylene)-, —X³—, or —X⁴—(C₁-C₆ alkylene)-, wherein: X³ and X⁴ are each independently selected from: —O—, —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; and w is 0, 1, or 2; provided that when L² is a bond, —(C₁-C₆ alkylene)-, —O—, or —O—(C₁-C₆ alkylene)- then R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂.

In some embodiments of a compound of Formula (VII), (VIIa), (VIIb), (VIIIa), (VIIIb), (VIIIc), or (VIIId), R¹³ is hydrogen, —(C₁-C₄ alkylene)-OH, or —(C₁-C₄ alkylene)-NH₂; each R¹⁵ and R¹⁶ is —F; R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R²⁰ groups, wherein; each R²⁰ is independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; L² is a —X³—, or —X⁴—(C₁-C₆ alkylene)-, wherein: X³ and X⁴ are each independently selected from: —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; and w is 0, 1, or 2.

In some embodiments of a compound of Formula (VII), (VIIa), (VIIb), (VIIIa), (VIIIb), (VIIIc), or (VIIId), R¹³ is hydrogen or —CH₂OH; each R¹⁵ and R¹⁶ is —F; R¹⁷ is 4- to 6-membered heterocycloalkyl, where the 4- to 6-membered heterocycloalkyl is unsubstituted or substituted by 1, 2, or 3 R²⁰ groups, wherein: each R²⁰ is independently selected from: —F, —OH, —OCH₃, and —NH; L² is a bond, —(C₁-C₆ alkylene)-, —X³—, or —X⁴—(C₁-C₆ alkylene)-, wherein: X³ and X⁴ are each independently selected from: —O—, —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; and w is 0, 1, or 2; provided that when L² is a bond, —(C₁-C₆ alkylene)-, —O—, or —O—(C₁-C₆ alkylene)- then R¹⁷ is an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R⁸)₂.

In some embodiments of a compound of Formula (VII), (VIIa), (VIIb), (VIIIa), (VIIIb), (VIIIc), or (VIIId), R¹³ is hydrogen or —CH₂OH; each R¹⁵ and R¹⁶ is —F; R¹⁷ is 4- to 6-membered heterocycloalkyl, where the 4- to 6-membered heterocycloalkyl is unsubstituted or substituted by 1, 2, or 3 R²⁰ groups, wherein: each R²⁰ is independently selected from: —F, —OH, —OCH₃, and —NH₂; L² is a —X³—, or —X⁴—(C₁-C₆ alkylene)-, wherein: X³ and X⁴ are each independently selected from: —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; and w is 0, 1, or 2.

In some embodiments, the compound is a compound of Formula (VIIIa), or a pharmaceutically acceptable salt, solvate, or stereoisomer thereof. In some embodiments, the compound is a compound of Formula (VIIIb), or a pharmaceutically acceptable salt, solvate, or stereoisomer thereof. In some embodiments, the compound is a compound of Formula (VIIIc), or a pharmaceutically acceptable salt, solvate, or stereoisomer thereof. In some embodiments, the compound is a compound of Formula (VIIId), or a pharmaceutically acceptable salt, solvate, or stereoisomer thereof.

In some embodiments of a compound of Formula (VI), (VIa), (VIb), (VII), (VIIa), (VIIb), (VIIIa), (VIIIb), (VIIIc), or (VIIId), L² is a bond, —(C₁-C₆ alkylene)-, —X³—, or —X⁴—(C₁-C₆ alkylene)-, wherein: X³ and X⁴ are each selected from: —O—, —N(R¹⁹)—, —S—, —S(═O)—, —S(═O)₂—, and —S(═O)(═NR¹⁹)—, wherein: R¹⁹ is hydrogen or C₁-C₆ alkyl. In some embodiments, L² is a bond, —(C₁-C₃ alkylene)-, —X³—, or —X⁴—(C₁-C₆ alkylene)-, wherein: X³ and X⁴ are each selected from: —O—, —N(R¹⁹)—, —S—, —S(═O)—, —S(═O)₂—, and —S(═O)(═NR¹⁹)—. In some embodiments, L² is a bond, —(C₁-C₂ alkylene)-, —X³—, or —X⁴—(C₁-C₆ alkylene)-, wherein: X³ and X⁴ are each selected from: —O—, —N(R¹⁹)—, —S—, —S(═O)—, —S(═O)₂—, and —S(═O)(═NR¹⁹)—. In some embodiments, L² is a bond, —CH₂—, —X³—, or —X⁴—(C₁-C₆ alkylene)-, wherein: X³ and X⁴ are each selected from: —O—, —N(R¹⁹)—, —S—, —S(═O)—, —S(═O)₂—, and —S(═O)(═NR¹⁹)—. In some embodiments, each R¹⁹ is independently hydrogen or methyl. In some embodiments, R¹⁹ is hydrogen.

In some embodiments of a compound of Formula (VI), (VIa), (VIb), (VII), (VIIa), (VIIb), (VIIIa), (VIIIb), (VIIIc), or (VIIId), L² is —(C₁-C₆ alkylene)-, —X³—, or —X⁴—(C₁-C₆ alkylene)-, wherein: X³ and X⁴ are each selected from: —O—, —N(R¹⁹)—, —S—, —S(═O)—, —S(═O)₂—, and —S(═O)(═NR¹⁹)—. In some embodiments, L² is —CH₂—, —X³—, or —X⁴—(C₁-C₆ alkylene)-, wherein: X³ and X⁴ are each selected from: —O—, —N(R¹⁹)—, —S—, —S(═O)—, —S(═O)₂—, and —S(═O)(═NR¹⁹)—. In some embodiments, L² is a bond, —X³—, or —X⁴—(C₁-C₆ alkylene)-, wherein: X³ and X⁴ are each selected from: —O—, —N(R¹⁹)—, —S—, —S(═O)—, —S(═O)₂—, and —S(═O)(═NR¹⁹)—. In some embodiments, R¹⁹ is hydrogen.

In some embodiments of a compound of Formula (VI), (VIa), (VIb), (VII), (VIIa), (VIIb), (VIIIa), (VIIIb), (VIIIc), or (VIIId), L² is a —X³—, or —X⁴—(C₁-C₆ alkylene)-, wherein: X³ and X⁴ are each selected from: —O—, —N(R¹⁹)—, —S—, —S(═O)—, —S(═O)₂—, and —S(═O)(═NR¹⁹)—. In some embodiments, L² is a —X³—, or —X⁴—(C₁-C₆ alkylene)-, wherein: X³ and X⁴ are each selected from: —O—, —N(R¹⁹)—, —S—, —S(═O)—, and —S(═O)₂—. In some embodiments, L² is a —X³—, or —X⁴—(C₁-C₆ alkylene)-, wherein: X³ and X⁴ are each selected from: —O—, —N(R¹⁹)— and —S—. In some embodiments, L² is a —X³—, or —X⁴—(C₁-C₆ alkylene)-, wherein: X³ and X⁴ are each selected from: —O— and —N(R¹⁹)—. In some embodiments, L² is a —X³—, or —X⁴—(C₁-C₆ alkylene)-, wherein: X³ and X⁴ are each selected from: —O— and —S—. In some embodiments, L² is a —X³—, or —X⁴—(C₁-C₆ alkylene)-, wherein: X³ and X⁴ are each selected from: —N(R¹⁹)— and —S—. In some embodiments, R¹⁹ is hydrogen.

In some embodiments of a compound of Formula (VI), (VIa), (VIb), (VII), (VIIa), (VIIb), (VIIIa), (VIIIb), (VIIIc), or (VIIId), L² is —X³— or —X⁴—(C₁-C₄ alkylene)-, wherein: X³ and X⁴ are each selected from: —O—, —N(H)—, —S—, —S(═O)—, —S(═O)₂—, and —S(═O)(═NH)—. In some embodiments, L² is —X³— or —X⁴—(C₁-C₄ alkylene)-, wherein: X³ and X⁴ are each selected from: —O—. In some embodiments, L² is —O—.

In some embodiments of a compound of Formula (VI), (VIa), (VIb), (VII), (VIIa), (VIIb), (VIIIa), (VIIIb), (VIIIc), or (VIIId), L² is —O— or —O—(C₁-C₆ alkylene)-. In some embodiments, L² is —N(R¹⁹)— or —N(R¹⁹)—(C₁-C₆ alkylene)-. In some embodiments, L² is —S— or —S—(C₁-C₆ alkylene)-. In some embodiments, L² is —S(═O)— or —S(═O)—(C₁-C₆ alkylene)-. In some embodiments, L² is —S(═O)₂— or —S(═O)₂—(C₁-C₆ alkylene)-. In some embodiments, L² is —S(═O)(═NR¹⁹)— or —S(═O)(═NR¹⁹)—(C₁-C₆ alkylene)-. In some embodiments, L² is —O—. In some embodiments, L² is —N(R¹⁹)—. In some embodiments, R¹⁹ is hydrogen. In some embodiments, L² is —S—. In some embodiments, L² is —S(═O)—. In some embodiments, L² is —S(═O)₂—. In some embodiments, L² is —S(═O)(═NR¹⁹)—. In some embodiments, L² is —O—(C₁-C₆ alkylene)-. In some embodiments, L² is —N(R¹⁹)—(C₁-C₆ alkylene)-. In some embodiments, L² is —S—(C₁-C₆ alkylene)-. In some embodiments, L² is —S(═O)—(C₁-C₆ alkylene)-. In some embodiments, L² is —S(═O)₂—(C₁-C₆ alkylene)-. In some embodiments, L² is —S(═O)(═NR¹⁹)—(C₁-C₆ alkylene)-. In some embodiments, L² is —O— or —O—(CH₂)—. In some embodiments, L² is —N(H)— or —N(H)—(CH₂)—. In some embodiments, L² is —S— or —S—(CH₂)—. In some embodiments, L² is —S(═O)— or —S(═O)—(CH₂)—. In some embodiments, L² is —S(═O)₂— or —S(═O)₂—(CH₂)—. In some embodiments, L² is —S(═O)(═NH)— or —S(═O)(═NH)—(CH₂)—. In some embodiments, L² is —O—(CH₂)—. In some embodiments, L² is —N(H)—(CH₂)—. In some embodiments, L² is —S—(CH₂)—. In some embodiments, L² is —S(═O)—(CH₂)—. In some embodiments, L² is —S(═O)₂—(CH₂)—. In some embodiments, L² is —S(═O)(═NH)—(CH₂)—.

In some embodiments of a compound of Formula (VI), (VIa), (VIb), (VII), (VIIa), (VIIb), (VIIIa), (VIIIb), (VIIIc), or (VIIId), In some embodiments, R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R²⁰ groups, wherein: each R²⁰ is independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃.

In some embodiments of a compound of Formula (VI), (VIa), (VIb), (VII), (VIIa), (VIIb), (VIIIa), (VIIIb), (VIIIc), or (VIIId), R¹⁷ is 4- to 8-membered heterocycloalkyl, where the 4- to 8-membered heterocycloalkyl is unsubstituted or substituted by 1, 2, or 3 R²⁰ groups, wherein: each R²⁰ is independently selected from: halogen, —OR¹⁸, —N(R¹⁸)₂, —NHSO₂R¹⁸, —CH₂CN, C₁-C₄ alkyl, —C(═O)—C₁-C₄ hydroxyalkyl, C₁-C₄ hydroxyalkyl, and C₁-C₄ methoxyalkyl; and each R¹⁸ is independently hydrogen, C₁-C₄ alkyl, —C(═O)—C₁-C₄ alkyl, or 4- to 6-membered heterocycloalkyl, wherein the alkyl is unsubstituted or substituted by 1, 2, or 3 groups independently selected from —F, —CN, —OH, —CO₂H, —C(═NH)NH₂, and 5-membered monocyclic heteroaryl which is unsubstituted or substituted by 1 —CONH₂ group.

In some embodiments of a compound of Formula (VI), (VIa), (VIb), (VII), (VIIa), (VIIb), (VIIIa), (VIIIb), (VIIIc), or (VIIId), R¹⁷ is 4- to 8-membered heterocycloalkyl, where the 4- to 8-membered heterocycloalkyl is unsubstituted or substituted by 1, 2, or 3 R²⁰ groups, wherein: each R²⁰ is independently selected from: halogen, —OH, —OMe, —N(R¹⁸)₂, —NHSO₂R¹⁸, and —CH₂CN; and each R¹⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl. In some embodiments, R¹⁷ is 4- to 8-membered heterocycloalkyl, where the 4- to 8-membered heterocycloalkyl is unsubstituted or substituted by 1, 2, or 3 R²⁰ groups, wherein: each R²⁰ is independently selected from: halogen, —OH, —OR¹⁸, —N(R¹⁸)₂, and —CH₂CN; and each R¹⁸ is independently hydrogen, C₁-C₂ alkyl, or —C(═O)—C₁-C₂ alkyl. In some embodiments, R¹⁷ is 4- to 8-membered heterocycloalkyl, where the 4- to 8-membered heterocycloalkyl is unsubstituted or substituted by 1, 2, or 3 R²⁰ groups, wherein: each R²⁰ is independently selected from: halogen, —OH, —OR¹⁸, —N(R¹⁸)₂, and —CH₂CN; and each R¹⁸ is independently hydrogen or C₁-C₂ alkyl.

In some embodiments of a compound of Formula (VI), (VIa), (VIb), (VII), (VIIa), (VIIb), (VIIIa), (VIIIb), (VIIIc), or (VIIId), R¹⁷ is 4- to 8-membered heterocycloalkyl, where the 4- to 8-membered heterocycloalkyl is unsubstituted or substituted by 1, 2, or 3 R²⁰ groups, wherein: each R²⁰ is independently selected from: halogen, —OH, —NH₂, and —CH₂CN. In some embodiments, R¹⁷ is 4- to 8-membered heterocycloalkyl, where the 4- to 8-membered heterocycloalkyl is unsubstituted or substituted by 1, 2, or 3 R²⁰ groups, wherein: each R²⁰ is independently selected from: halogen and —OH. In some embodiments, R¹⁷ is 4- to 8-membered heterocycloalkyl, where the 4- to 8-membered heterocycloalkyl is unsubstituted or substituted by 1, 2, or 3 R²⁰ groups, wherein: each R²⁰ is independently selected from: —F, —Cl, and —OH. In some embodiments, R¹⁷ is 4- to 8-membered heterocycloalkyl, where the 4- to 8-membered heterocycloalkyl is unsubstituted or substituted by 1, 2, or 3 R²⁰ groups, wherein: each R²⁰ is independently selected from: —F and —OH.

In some embodiments, R¹⁷ is 4- to 6-membered heterocycloalkyl, where the 4- to 6-membered heterocycloalkyl is unsubstituted or substituted by 1, 2, or 3 R²⁰ groups, wherein: each R²⁰ is independently selected from: halogen, —OH, —OMe, —N(R¹⁸)₂, —NHSO₂R¹⁸, and —CH₂CN; and each R¹⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl. In some embodiments, R¹⁷ is 4- to 6-membered heterocycloalkyl, where the 4- to 6-membered heterocycloalkyl is unsubstituted or substituted by 1, 2, or 3 R²⁰ groups, wherein: each R²⁰ is independently selected from: halogen, —OH, —OR¹⁸, —N(R¹⁸)₂, and —CH₂CN; and each R¹⁸ is independently hydrogen, C₁-C₂ alkyl, or —C(═O)—C₁-C₂ alkyl. In some embodiments, R¹⁷ is 4- to 6-membered heterocycloalkyl, where the 4- to 6-membered heterocycloalkyl is unsubstituted or substituted by 1, 2, or 3 R²⁰ groups, wherein: each R²⁰ is independently selected from: halogen, —OH, —OR¹⁸, —N(R¹⁸)₂, and —CH₂CN; and each R¹⁸ is independently hydrogen or C₁-C₂ alkyl.

In some embodiments of a compound of Formula (VI), (VIa), (VIb), (VII), (VIIa), (VIIb), (VIIIa), (VIIIb), (VIIIc), or (VIIId), R¹⁷ is 4- to 6-membered heterocycloalkyl, where the 4- to 6-membered heterocycloalkyl is unsubstituted or substituted by 1, 2, or 3 R²⁰ groups, wherein: each R²⁰ is independently selected from: halogen, —OH, —NH₂, and —CH₂CN. In some embodiments, R¹⁷ is 4- to 6-membered heterocycloalkyl, where the 4- to 6-membered heterocycloalkyl is unsubstituted or substituted by 1, 2, or 3 R²⁰ groups, wherein: each R²⁰ is independently selected from: halogen and —OH. In some embodiments, R¹⁷ is 4- to 6-membered heterocycloalkyl, where the 4- to 6-membered heterocycloalkyl is unsubstituted or substituted by 1, 2, or 3 R²⁰ groups, wherein: each R²⁰ is independently selected from: —F, —Cl, and —OH. In some embodiments, R¹⁷ is 4- to 6-membered heterocycloalkyl, where the 4- to 6-membered heterocycloalkyl is unsubstituted or substituted by 1, 2, or 3 R²⁰ groups, wherein: each R²⁰ is independently selected from: —F and —OH.

In some embodiments of a compound of Formula (VI), (VIa), (VIb), (VII), (VIIa), (VIIb), (VIIIa), (VIIIb), (VIIIc), or (VIIId), R¹⁷ is 5- to 6-membered heterocycloalkyl, where the 5- to 6-membered heterocycloalkyl is unsubstituted or substituted by 1, 2, or 3 R²⁰ groups, wherein: each R²⁰ is independently selected from: halogen, —OH, —OMe, —N(R¹⁸)₂, —NHSO₂R¹⁸, and —CH₂CN; and each R¹⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl. In some embodiments, R¹⁷ is 5- to 6-membered heterocycloalkyl, where the 5- to 6-membered heterocycloalkyl is unsubstituted or substituted by 1, 2, or 3 R²⁰ groups, wherein: each R²⁰ is independently selected from: halogen, —OH, —OR¹⁸, —N(R¹⁸)₂, and —CH₂CN; and each R¹⁸ is independently hydrogen, C₁-C₂ alkyl, or —C(═O)—C₁-C₂ alkyl. In some embodiments, R¹⁷ is 5- to 6-membered heterocycloalkyl, where the 5- to 6-membered heterocycloalkyl is unsubstituted or substituted by 1, 2, or 3 R²⁰ groups, wherein: each R²⁰ is independently selected from: halogen, —OH, —OR¹⁸, —N(R¹⁸)₂, and —CH₂CN; and each R¹⁸ is independently hydrogen or C₁-C₂ alkyl.

In some embodiments of a compound of Formula (VI), (VIa), (VIb), (VII), (VIIa), (VIIb), (VIIIa), (VIIIb), (VIIIc), or (VIIId), R¹⁷ is 5- to 6-membered heterocycloalkyl, where the 5- to 6-membered heterocycloalkyl is unsubstituted or substituted by 1, 2, or 3 R²⁰ groups, wherein: each R²⁰ is independently selected from: halogen, —OH, —NH₂, and —CH₂CN. In some embodiments, R¹⁷ is 5- to 6-membered heterocycloalkyl, where the 5- to 6-membered heterocycloalkyl is unsubstituted or substituted by 1, 2, or 3 R²⁰ groups, wherein: each R²⁰ is independently selected from: halogen and —OH. In some embodiments, R¹⁷ is 5- to 6-membered heterocycloalkyl, where the 5- to 6-membered heterocycloalkyl is unsubstituted or substituted by 1, 2, or 3 R²⁰ groups, wherein: each R²⁰ is independently selected from: —F, —Cl, and —OH. In some embodiments, R¹⁷ is 5- to 6-membered heterocycloalkyl, where the 5- to 6-membered heterocycloalkyl is unsubstituted or substituted by 1, 2, or 3 R²⁰ groups, wherein: each R²⁰ is independently selected from: —F and —OH.

In some embodiments, R¹⁷ is 5-membered heterocycloalkyl, where the 5-membered heterocycloalkyl is unsubstituted or substituted by 1, 2, or 3 R²⁰ groups, wherein: each R²⁰ is independently selected from: halogen, —OH, —OMe, —N(R¹⁸)₂, —NHSO₂R¹⁸, and —CH₂CN; and each R¹⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl. In some embodiments, R¹⁷ is 5-membered heterocycloalkyl, where the 5-membered heterocycloalkyl is unsubstituted or substituted by 1, 2, or 3 R²⁰ groups, wherein: each R²⁰ is independently selected from: halogen, —OH, —OR¹⁸, —N(R¹⁸)₂, and —CH₂CN; and each R¹⁸ is independently hydrogen, C₁-C₂ alkyl, or —C(═O)—C₁-C₂ alkyl. In some embodiments, R¹⁷ is 5-membered heterocycloalkyl, where the 5-membered heterocycloalkyl is unsubstituted or substituted by 1, 2, or 3 R²⁰ groups, wherein: each R²⁰ is independently selected from: halogen, —OH, —OR¹⁸, —N(R¹⁸)₂, and —CH₂CN; and each R¹⁸ is independently hydrogen or C₁-C₂ alkyl.

In some embodiments of a compound of (VI), (VIa), (VIb), (VII), (VIIa), (VIIb), (VIIIa), (VIIIb), (VIIIc), or (VIIId), R¹⁷ is 5-membered heterocycloalkyl, where the 5-membered heterocycloalkyl is unsubstituted or substituted by 1, 2, or 3 R²⁰ groups, wherein: each R²⁰ is independently selected from: halogen, —OH, —NH₂, and —CH₂CN. In some embodiments, R¹⁷ is 5-membered heterocycloalkyl, where the 5-membered heterocycloalkyl is unsubstituted or substituted by 1, 2, or 3 R²⁰ groups, wherein: each R²⁰ is independently selected from: halogen and —OH. In some embodiments, R¹⁷ is 5-membered heterocycloalkyl, where the 5-membered heterocycloalkyl is unsubstituted or substituted by 1, 2, or 3 R²⁰ groups, wherein: each R²⁰ is independently selected from: —F, —Cl, and —OH. In some embodiments, R¹⁷ is 5-membered heterocycloalkyl, where the 5-membered heterocycloalkyl is unsubstituted or substituted by 1, 2, or 3 R²⁰ groups, wherein: each R²⁰ is independently selected from: —F and —OH.

In some embodiments, R¹⁷ is tetrahydrofuran, where the tetrahydrofuran is unsubstituted or substituted by 1, 2, or 3 R²⁰ groups, wherein: each R²⁰ is independently selected from: halogen, —OH, —OMe, —N(R¹⁸)₂, —NHSO₂R¹⁸, and —CH₂CN; and each R¹⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl. In some embodiments, R¹⁷ is tetrahydrofuran, where the tetrahydrofuran is unsubstituted or substituted by 1, 2, or 3 R²⁰ groups, wherein: each R²⁰ is independently selected from: halogen, —OH, —OR¹⁸, —N(R¹⁸)₂, and —CH₂CN; and each R¹⁸ is independently hydrogen, C₁-C₂ alkyl, or —C(═O)—C₁-C₂ alkyl. In some embodiments, R¹⁷ is tetrahydrofuran, where the tetrahydrofuran is unsubstituted or substituted by 1, 2, or 3 R²⁰ groups, wherein: each R²⁰ is independently selected from: halogen, —OH, —OR¹⁸, —N(R¹⁸)₂, and —CH₂CN; and each R¹⁸ is independently hydrogen or C₁-C₂ alkyl.

In some embodiments of a compound of Formula (VI), (VIa), (VIb), (VII), (VIIa), (VIIb), (VIIIa), (VIIIb), (VIIIc), or (VIIId), R¹⁷ is tetrahydrofuran, where the tetrahydrofuran is unsubstituted or substituted by 1, 2, or 3 R²⁰ groups, wherein: each R²⁰ is independently selected from: halogen, —OH, —NH₂, and —CH₂CN. In some embodiments, R¹⁷ is tetrahydrofuran, where the tetrahydrofuran is unsubstituted or substituted by 1, 2, or 3 R²⁰ groups, wherein: each R²⁰ is independently selected from: halogen and —OH. In some embodiments, R¹⁷ is tetrahydrofuran, where the tetrahydrofuran is unsubstituted or substituted by 1, 2, or 3 R²⁰ groups, wherein: each R²⁰ is independently selected from: —F, —Cl, and —OH. In some embodiments, R¹⁷ is tetrahydrofuran, where the tetrahydrofuran is unsubstituted or substituted by 1, 2, or 3 R²⁰ groups, wherein: each R²⁰ is independently selected from: —F and —OH. In some embodiments, R¹⁷ is tetrahydrofuran substituted by 3 R²⁰ groups, wherein: each R²⁰ is independently selected from: —F and —OH.

In some embodiments of a compound of Formula (VI), (VIa), (VIb), (VII), (VIIa), (VIIb), (VIIIa), (VIIIb), (VIIIc), or (VIIId), L² is a —X³—, or —X⁴—(C₁-C₆ alkylene)-, wherein: X³ and X⁴ are each selected from: —N(R¹⁹)—, —S—, —S(═O)—, —S(═O)₂—, and —S(═O)(═NR¹⁹)—. In some embodiments, L² is a —X³—, or —X⁴—(C₁-C₆ alkylene)-, wherein: X³ and X⁴ are each selected from: —N(R¹⁹)—, —S—, —S(═O)—, and —S(═O)₂—. In some embodiments, L² is a —X³—, or —X⁴—(C₁-C₆ alkylene)-, wherein: X³ and X⁴ are each selected from: —N(R¹⁹)— and —S—. In some embodiments, L² is a —X³—, or —X⁴—(C₁-C₆ alkylene)-, wherein: X³ and X⁴ are each selected from: —S— and —N(R¹⁹)—. In some embodiments, L² is a —X³—, or —X⁴—(C₁-C₆ alkylene)-, wherein: X³ and X⁴ are each selected from: —S(═O)₂—, and —N(R¹⁹)—. In some embodiments, L² is a —X³—, or —X⁴—(C₁-C₆ alkylene)-, wherein: X³ and X⁴ are each selected from: —S(═O)(═NR¹⁹)— and —N(R¹⁹)—. In some embodiments, R¹⁹ is hydrogen.

In some embodiments of a compound of Formula (VI), (VIa), (VIb), (VII), (VIIa), (VIIb), (VIIIa), (VIIIb), (VIIIc), or (VIIId), L² is a —X³—, or —X⁴—(C₁-C₆ alkylene)-, wherein: X³ and X⁴ are each selected from: —N(R¹⁹)—, —S—, —S(═O)—, —S(═O)₂—, and —S(═O)(═NR¹⁹)—; and R¹⁷ is tetrahydrofuran, where the tetrahydrofuran is unsubstituted or substituted by 1, 2, or 3 R²⁰ groups, wherein: each R²⁰ is independently selected from: halogen, —OH, —NH₂, and —CH₂CN. In some embodiments, L² is a —X³—, or —X⁴—(C₁-C₆ alkylene)-, wherein: X³ and X⁴ are each selected from: —N(R¹⁹)—, —S—, —S(═O)—, —S(═O)₂—, and —S(═O)(═NR¹⁹)—; and R¹⁷ is tetrahydrofuran, where the tetrahydrofuran is unsubstituted or substituted by 1, 2, or 3 R²⁰ groups, wherein: each R²⁰ is independently selected from: halogen and —OH. In some embodiments, L² is a —X³—, or —X⁴—(C₁-C₆ alkylene)-, wherein: X³ and X⁴ are each selected from: —N(R¹⁹)—, —S—, —S(═O)—, —S(═O)₂—, and —S(═O)(═NR¹⁹)—; and R¹⁷ is tetrahydrofuran, where the tetrahydrofuran is unsubstituted or substituted by 1, 2, or 3 R²⁰ groups, wherein: each R²⁰ is independently selected from: —F, —Cl, and —OH. In some embodiments, L² is a —X³—, or —X⁴—(C₁-C₆ alkylene)-, wherein: X³ and X⁴ are each selected from: —N(R¹⁹)—, —S—, —S(═O)—, —S(═O)₂—, and —S(═O)(═NR¹⁹)—; and R¹⁷ is tetrahydrofuran, where the tetrahydrofuran is unsubstituted or substituted by 1, 2, or 3 R²⁰ groups, wherein: each R²⁰ is independently selected from: —F and —OH. In some embodiments, L² is a —X³—, or —X⁴—(C₁-C₆ alkylene)-, wherein: X³ and X⁴ are each selected from: —N(R¹⁹)—, —S—, —S(═O)—, —S(═O)₂—, and —S(═O)(═NR¹⁹)—; and R¹⁷ is tetrahydrofuran substituted by 1, 2, or 3 R²⁰ groups, wherein: each R²⁰ is independently selected from: —F and —OH.

In some embodiments of a compound of Formula (VI), (VIa), (VIb), (VII), (VIIa), (VIIb), (VIIIa), (VIIIb), (VIIIc), or (VIIId), L² is selected from: —N(R¹⁹)—, —S—, —S(═O)—, —S(═O)₂—, and —S(═O)(═NR¹⁹)—; and R¹⁷ is tetrahydrofuran, where the tetrahydrofuran is unsubstituted or substituted by 1, 2, or 3 R²⁰ groups, wherein: each R²⁰ is independently selected from: halogen, —OH, —NH₂, and —CH₂CN. In some embodiments, L² is selected from: —N(R¹⁹)—, —S—, —S(═O)—, —S(═O)₂—, and —S(═O)(═NR¹⁹)—; and R¹⁷ is tetrahydrofuran, where the tetrahydrofuran is unsubstituted or substituted by 1, 2, or 3 R²⁰ groups, wherein: each R²⁰ is independently selected from: halogen and —OH. In some embodiments, L² is selected from: —N(R¹⁹)—, —S—, —S(═O)—, —S(═O)₂—, and —S(═O)(═NR¹⁹)—; and R¹⁷ is tetrahydrofuran, where the tetrahydrofuran is unsubstituted or substituted by 1, 2, or 3 R²⁰ groups, wherein: each R²⁰ is independently selected from: —F, —Cl, and —OH. In some embodiments, L² is selected from: —N(R¹⁹)—, —S—, —S(═O)—, —S(═O)₂—, and —S(═O)(═NR¹⁹)—; and R¹⁷ is tetrahydrofuran, where the tetrahydrofuran is unsubstituted or substituted by 1, 2, or 3 R²⁰ groups, wherein: each R²⁰ is independently selected from: —F and —OH. In some embodiments, L² is selected from: —N(R¹⁹)—, —S—, —S(═O)—, —S(═O)₂—, and —S(═O)(═NR¹⁹)—; and R¹⁷ is tetrahydrofuran substituted by 1, 2, or 3 R²⁰ groups, wherein: each R²⁰ is independently selected from: —F and —OH.

In some embodiments of a compound of Formula (VI), (VIa), (VIb), (VII), (VIIa), (VIIb), (VIIIa), (VIIIb), (VIIIc), or (VIIId), L² is selected from: —N(R¹⁹)— and —S—; and R¹⁷ is tetrahydrofuran, where the tetrahydrofuran is unsubstituted or substituted by 1, 2, or 3 R²⁰ groups, wherein: each R²⁰ is independently selected from: halogen, —OH, —NH₂, and —CH₂CN. In some embodiments, L² is selected from: —N(R¹⁹)— and —S—; and R¹⁷ is tetrahydrofuran, where the tetrahydrofuran is unsubstituted or substituted by 1, 2, or 3 R²⁰ groups, wherein: each R²⁰ is independently selected from: halogen and —OH. In some embodiments, L² is selected from: —N(R¹⁹)— and —S—; and R¹⁷ is tetrahydrofuran, where the tetrahydrofuran is unsubstituted or substituted by 1, 2, or 3 R²⁰ groups, wherein: each R²⁰ is independently selected from: —F, —Cl, and —OH. In some embodiments, L² is selected from: —N(R¹⁹)— and —S—; and R¹⁷ is tetrahydrofuran, where the tetrahydrofuran is unsubstituted or substituted by 1, 2, or 3 R²⁰ groups, wherein: each R²⁰ is independently selected from: —F and —OH. In some embodiments, L² is selected from: —N(R¹⁹)— and —S—; and R¹⁷ is tetrahydrofuran substituted by 1, 2, or 3 R²⁰ groups, wherein: each R²⁰ is independently selected from: —F and —OH.

In some embodiments of a compound of Formula (VI), (VIa), (VIb), (VII), (VIIa), (VIIb), (VIIIa), (VIIIb), (VIIIc), or (VIIId), L² is selected from: —N(R¹⁹)— and —N(R¹⁹)—(C₁-C₆ alkylene)-; and R¹⁷ is tetrahydrofuran, where the tetrahydrofuran is unsubstituted or substituted by 1, 2, or 3 R²⁰ groups, wherein: each R²⁰ is independently selected from: halogen, —OH, —NH₂, and —CH₂CN. In some embodiments, L² is selected from: —N(R¹⁹)— and —N(R¹⁹)—(C₁-C₆ alkylene); and R¹⁷ is tetrahydrofuran, where the tetrahydrofuran is unsubstituted or substituted by 1, 2, or 3 R²⁰ groups, wherein: each R²⁰ is independently selected from: halogen and —OH. In some embodiments, L² is selected from: —N(R¹⁹)— and —N(R¹⁹)—(C₁-C₆ alkylene)-; and R¹⁷ is tetrahydrofuran, where the tetrahydrofuran is unsubstituted or substituted by 1, 2, or 3 R²⁰ groups, wherein: each R²⁰ is independently selected from: —F, —Cl, and —OH. In some embodiments, L² is selected from: —N(R¹⁹)— and —N(R¹⁹)—(C₁-C₆ alkylene)-; and R¹⁷ is tetrahydrofuran, where the tetrahydrofuran is unsubstituted or substituted by 1, 2, or 3 R²⁰ groups, wherein: each R²⁰ is independently selected from: —F and —OH. In some embodiments, L² is selected from: —N(R¹⁹)— and —N(R¹⁹)—(C₁-C₆ alkylene)-; and R¹⁷ is tetrahydrofuran substituted by 1, 2, or 3 R²⁰ groups, wherein: each R²⁰ is independently selected from: —F and —OH.

In some embodiments of a compound of Formula (VI), (VIa), (VIb), (VII), (VIIa), (VIIb), (VIIIa), (VIIIb), (VIIIc), or (VIIId), L² is —N(R¹⁹)— and R¹⁷ is azetidine, where the azetidine is unsubstituted or substituted by 1, 2, or 3 R²⁰ groups, wherein: each R²⁰ is independently selected from: halogen, —OH, —NH₂, and —CH₂CN. In some embodiments, L² is —N(R¹⁹)— and R¹⁷ is azetidine, where the azetidine is unsubstituted or substituted by 1, 2, or 3 R²⁰ groups, wherein: each R²⁰ is independently selected from: halogen and —CH₂CN. In some embodiments, L² is —N(R¹⁹)— and R¹⁷ is azetidine, where the azetidine is unsubstituted or substituted by 1, 2, or 3 R²⁰ groups, wherein: each R²⁰ is independently selected from: —F, —Cl, and —CH₂CN. In some embodiments, L² is —N(R¹⁹)— and R¹⁷ is azetidine, where the azetidine is unsubstituted or substituted by 1, 2, or 3 R²⁰ groups, wherein: each R²⁰ is —CH₂CN.

In some embodiments of a compound of Formula (VI), (VIa), (VIb), (VII), (VIIa), (VIIb), (VIIIa), (VIIIb), (VIIIc), or (VIIId), L² is —N(R¹⁹)— and R¹⁷ is cyclobutane, where the cyclobutane is unsubstituted or substituted with C₁-C₆ alkyl, wherein C₁-C₆ alkyl is substituted with halogen, —OR¹⁸, —N(R¹⁸)₂. In some embodiments, L² is —N(R¹⁹)— and R¹⁷ is cyclobutane, where the cyclobutane is unsubstituted or substituted with C₁-C₆ alkyl, wherein C₁-C₆ alkyl is substituted with halogen or —N(R¹⁸)₂. In some embodiments, L² is —N(R¹⁹)— and R¹⁷ is cyclobutane, where the cyclobutane is unsubstituted or substituted with C₁-C₆ alkyl, wherein C₁-C₆ alkyl is substituted with halogen or —N(H)(CH₂CN). In some embodiments, L² is —N(R¹⁹)— and R¹⁷ is cyclobutane, where the cyclobutane is substituted with C₁-C₆ alkyl, wherein C₁-C₆ alkyl is substituted with halogen or —N(H)(CH₂CN).

In some embodiments of a compound of Formula (VI), (VIa), (VIb), (VII), (VIIa), (VIIb), (VIIIa), (VIIIb), (VIIIc), or (VIIId), L²-R¹⁷ is

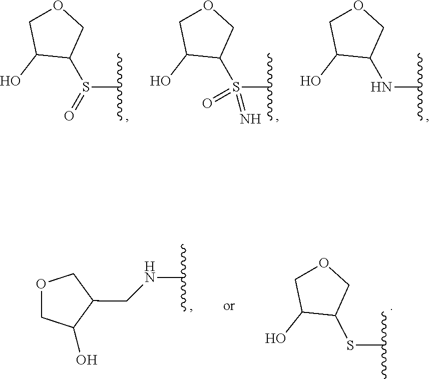 [SMILES: *S(=O)C1COCC1O ; *S(=N)(=O)C1COCC1O ; *NC1COCC1O ; *NCC1COCC1O ; *SC1COCC1O]

In some embodiments of a compound of (VI), (VIa), (VIb), (VII), (VIIa), (VIIb), (VIIIa), (VIIIb), (VIIIc), or (VIIId), L²-R¹⁷ is

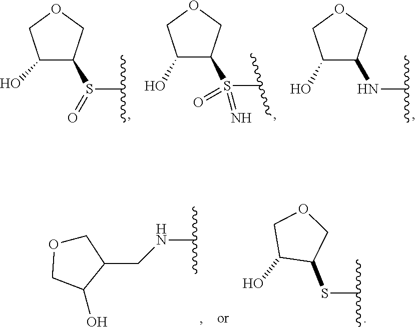

In some embodiments of a compound of Formula (VI), (VIa), (VIb), (VII), (VIIa), (VIIb), (VIIIa), (VIIIb), (VIIIc), or (VIIId), L²-R¹⁷ is

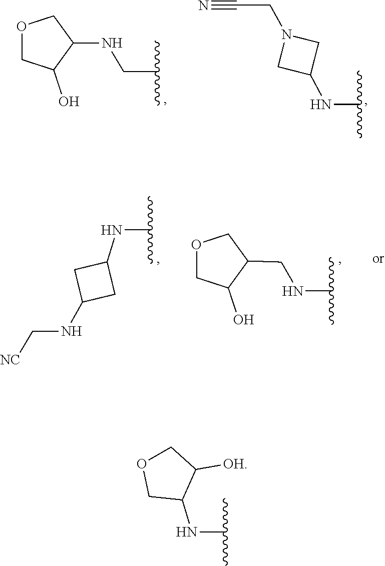 [SMILES: *CNC1COCC1O ; *NC1CN(CC#N)C1 ; *NC1CC(NCC#N)C1 ; *NCC1COCC1O ; *NC1COCC1O]

In some embodiments of a compound of Formula (VI), (VIa), (VIb), (VII), (VIIa), (VIIb), (VIIIa), (VIIIb), (VIIIc), or (VIIId), L²-R¹⁷ is

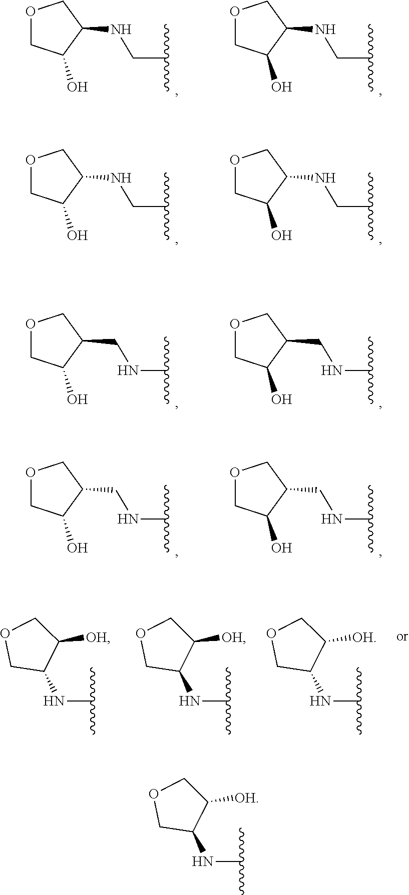

In some embodiments of a compound of Formula (VI), (VIa), (VIb), (VII), (VIIa), (VIIb), (VIIIa), (VIIIb), (VIIIc), or (VIIId), R¹³ is —(C₃-C₆ cycloalkylene)-OH or —(C₃-C₆ cycloalkylene)-NH₂; L² is —O—; and R¹⁷ is tetrahydrofuran, where the tetrahydrofuran is unsubstituted or substituted by 1, 2, or 3 R²⁰ groups, wherein: each R²⁰ is independently selected from: halogen, —OH, —NH₂, and —CH₂CN. In some embodiments, R¹³ is —(C₃-C₆ cycloalkylene)-OH or —(C₃-C₆ cycloalkylene)-NH₂; L² is —O—; and R¹⁷ is tetrahydrofuran, where the tetrahydrofuran is unsubstituted or substituted by 1, 2, or 3 R²⁰ groups, wherein: each R²⁰ is independently selected from: halogen and —OH. In some embodiments, R¹³ is —(C₃-C₆ cycloalkylene)-OH or —(C₃-C₆ cycloalkylene)-NH₂; L² is —O—; and R¹⁷ is tetrahydrofuran, where the tetrahydrofuran is unsubstituted or substituted by 1, 2, or 3 R²⁰ groups, wherein: each R²⁰ is independently selected from: —F, —Cl, and —OH. In some embodiments, R¹³ is —(C₃-C₆ cycloalkylene)-OH or —(C₃-C₆ cycloalkylene)-NH₂; L² is —O—; and R¹⁷ is tetrahydrofuran, where the tetrahydrofuran is unsubstituted or substituted by 1, 2, or 3 R²⁰ groups, wherein: each R²⁰ is independently selected from: —F and —OH. In some embodiments, R¹³ is —(C₃-C₆ cycloalkylene)-OH or —(C₃-C₆ cycloalkylene)-NH₂; L² is —O—; and R¹⁷ is tetrahydrofuran substituted by 1, 2, or 3 R²⁰ groups, wherein: each R²⁰ is independently selected from: —F and —OH.

In some embodiments of a compound of Formula (VI), (VIa), (VIb), (VII), (VIIa), (VIIb), (VIIIa), (VIIIb), (VIIIc), or (VIIId), R¹⁴ is —CH₃; L² is —O—; and R¹⁷ is tetrahydrofuran, where the tetrahydrofuran is unsubstituted or substituted by 1, 2, or 3 R²⁰ groups, wherein: each R²⁰ is independently selected from: halogen, —OH, —NH₂, and —CH₂CN. In some embodiments, R¹³ is

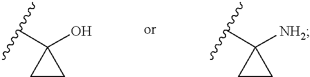 [SMILES: *C1(O)CC1 ; "or" ; *C1(N)CC1]

L² is —O—; and R¹⁷ is tetrahydrofuran, where the tetrahydrofuran is unsubstituted or substituted by 1, 2, or 3 R²⁰ groups, wherein: each R²⁰ is independently selected from: halogen and —OH. In some embodiments, R¹³ is

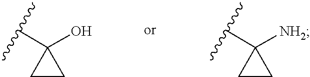 [SMILES: *C1(O)CC1 ; "or" ; *C1(N)CC1]

L² is —O—; and R¹⁷ is tetrahydrofuran, where the tetrahydrofuran is unsubstituted or substituted by 1, 2, or 3 R²⁰ groups, wherein: each R²⁰ is independently selected from: —F, —Cl, and —OH. In some embodiments, R¹³ is

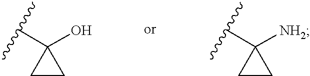 [SMILES: *C1(O)CC1 ; "or" ; *C1(N)CC1]

L² is —O—; and R¹⁷ is tetrahydrofuran, where the tetrahydrofuran is unsubstituted or substituted by 1, 2, or 3 R²⁰ groups, wherein: each R²⁰ is independently selected from: —F and —OH. In some embodiments, R¹³ is

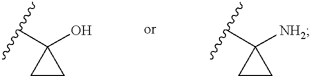 [SMILES: *C1(O)CC1 ; "or" ; *C1(N)CC1]

L² is —O—; and R¹⁷ is tetrahydrofuran substituted by 1, 2, or 3 R²⁰ groups, wherein: each R²⁰ is independently selected from: —F and —OH.

In some embodiments of a compound of Formula (VI), (VIa), (VIb), (VII), (VIIa), (VIIb), (VIIIa), (VIIIb), (VIIIc), or (VIIId), R¹⁴ is C₁-C₄ alkyl; and L²-R¹⁷ is

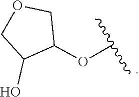 [SMILES: *OC1COCC1O]

In some embodiments, R¹⁴ is C₁-C₄ alkyl; and L²-R¹⁷ is

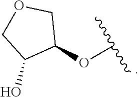 [SMILES: *O[C@@H]1COC[C@H]1O]

In some embodiments, R¹³ is

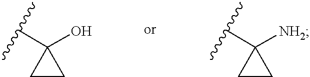 [SMILES: *C1(O)CC1 ; "or" ; *C1(N)CC1]

and L²-R¹⁷ is

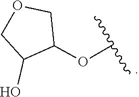 [SMILES: *OC1COCC1O]

In some embodiments, R¹³ is

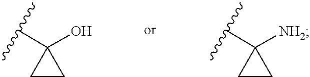 [SMILES: *C1(O)CC1 ; "or" ; *C1(N)CC1]

and L²-R¹⁷ is

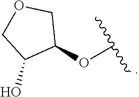 [SMILES: *O[C@@H]1COC[C@H]1O]

In some embodiments of a compound of Formula (VI), (VIa), (VIb), (VII), (VIIa), (VIIb), (VIIIa), (VIIIb), (VIIIc), or (VIIId), R¹⁴ is C₁-C₄ alkyl; L² is —O—; and R¹⁷ is tetrahydrofuran, where the tetrahydrofuran is unsubstituted or substituted by 1, 2, or 3 R²⁰ groups, wherein: each R²⁰ is independently selected from: halogen, —OH, —NH₂, and —CH₂CN. In some embodiments, L² is R¹⁴ is C₁-C₄ alkyl; L² is —O—; and R¹⁷ is tetrahydrofuran, where the tetrahydrofuran is unsubstituted or substituted by 1, 2, or 3 R²⁰ groups, wherein: each R²⁰ is independently selected from: halogen and —OH. In some embodiments, R¹⁴ is C₁-C₄ alkyl; L² is —O—; and R¹⁷ is tetrahydrofuran, where the tetrahydrofuran is unsubstituted or substituted by 1, 2, or 3 R²⁰ groups, wherein: each R²⁰ is independently selected from: —F, —Cl, and —OH. In some embodiments, R¹⁴ is C₁-C₄ alkyl; L² is —O—; and R¹⁷ is tetrahydrofuran, where the tetrahydrofuran is unsubstituted or substituted by 1, 2, or 3 R²⁰ groups, wherein: each R²⁰ is independently selected from: —F and —OH. In some embodiments, R¹⁴ is C₁-C₄ alkyl; L² is —O—; and R¹⁷ is tetrahydrofuran substituted by 1, 2, or 3 R²⁰ groups, wherein: each R²⁰ is independently selected from: —F and —OH.

In some embodiments of a compound of Formula (VI), (VIa), (VIb), (VII), (VIIa), (VIIb), (VIIIa), (VIIIb), (VIIIc), or (VIIId), R¹⁴ is —CH₃; L² is —O—; and R¹⁷ is tetrahydrofuran, where the tetrahydrofuran is unsubstituted or substituted by 1, 2, or 3 R²⁰ groups, wherein: each R²⁰ is independently selected from: halogen, —OH, —NH₂, and —CH₂CN. In some embodiments, R¹⁴ is —CH₃; L² is —O—; and R¹⁷ is tetrahydrofuran, where the tetrahydrofuran is unsubstituted or substituted by 1, 2, or 3 R²⁰ groups, wherein: each R²⁰ is independently selected from: halogen and —OH. In some embodiments, R¹⁴ is —CH₃; L² is —O—; and R¹⁷ is tetrahydrofuran, where the tetrahydrofuran is unsubstituted or substituted by 1, 2, or 3 R²⁰ groups, wherein: each R²⁰ is independently selected from: —F, —Cl, and —OH. In some embodiments, R¹⁴ is —CH₃; L² is —O—; and R¹⁷ is tetrahydrofuran, where the tetrahydrofuran is unsubstituted or substituted by 1, 2, or 3 R²⁰ groups, wherein: each R²⁰ is independently selected from: —F and —OH. In some embodiments, R¹⁴ is —CH₃; L² is —O—; and R¹⁷ is tetrahydrofuran substituted by 1, 2, or 3 R²⁰ groups, wherein: each R²⁰ is independently selected from: —F and —OH.

In some embodiments of a compound of Formula (VI), (VIa), (VIb), (VII), (VIIa), (VIIb), (VIIIa), (VIIIb), (VIIIc), or (VIIId), is C₁-C₄ alkyl; and L²-R¹⁷ is

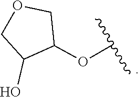 [SMILES: *OC1COCC1O]

In some embodiments, R¹⁴ is C₁-C₄ alkyl; and L²-R¹⁷ is

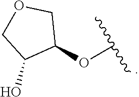 [SMILES: *O[C@@H]1COC[C@H]1O]

In some embodiments, R¹⁴ is —CH₃; and L²-R¹⁷ is

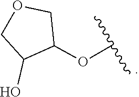 [SMILES: *OC1COCC1O]

In some embodiments, R¹⁴ is —CH₃; and L²-R¹⁷ is

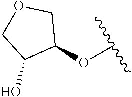 [SMILES: *O[C@@H]1COC[C@H]1O]

In some embodiments of a compound of Formula (VI), (VIa), (VIb), (VII), (VIIa), (VIIb), (VIIIa), (VIIIb), (VIIIc), or (VIIId), L² is a bond, —(C₁-C₆ alkylene)-, —O—, or —O—(C₁-C₆ alkylene)-; and R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂. In some embodiments, L² is a —O—, or —O—C₁-C₆ alkylene-, R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂.

In some embodiments of a compound of Formula (VI), (VIa), (VIb), (VII), (VIIa), (VIIb), (VIIIa), (VIIIb), (VIIIc), or (VIIId), L² is a bond, —CH₂—, —O—, or —O—CH₂—; and R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂. In some embodiments, L² is —O— or —O—CH₂—; R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂. In some embodiments, L² is —O—; and R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂.

In some embodiments of a compound of Formula (VI), (VIa), (VIb), (VII), (VIIa), (VIIb), (VIIIa), (VIIIb), (VIIIc), or (VIIId), L² is a bond, —(C₁-C₆ alkylene)-, —O—, or —O—(C₁-C₆ alkylene)-; and R¹⁷ is an unsubstituted or substituted hexahydrofuro[3,2-b]furan, an unsubstituted or substituted tetrahydrothiophene-1-oxide, an unsubstituted or substituted 3-oxabicyclo[3.1.0]hexane, an unsubstituted or substituted tetrahydropyran, a disubstituted or trisubstituted cyclopropyl, or an oxetane substituted by at least one —N(R¹⁸). In some embodiments, L² is a —O—, or —O—C₁-C₆ alkylene-, R¹⁷ is an unsubstituted or substituted hexahydrofuro[3,2-b]furan, an unsubstituted or substituted tetrahydrothiophene-1-oxide, an unsubstituted or substituted 3-oxabicyclo[3.1.0]hexane, an unsubstituted or substituted tetrahydropyran, a disubstituted or trisubstituted cyclopropyl, or an oxetane substituted by at least one —N(R¹⁸).

In some embodiments of a compound of Formula (VI), (VIa), (VIb), (VII), (VIIa), (VIIb), (VIIIa), (VIIIb), (VIIIc), or (VIIId), L² is a bond, —CH₂—, —O—, or —O—CH₂—; and R¹⁷ is an unsubstituted or substituted hexahydrofuro[3,2-b]furan, an unsubstituted or substituted tetrahydrothiophene-1-oxide, an unsubstituted or substituted 3-oxabicyclo[3.1.0]hexane, an unsubstituted or substituted tetrahydropyran, a disubstituted or trisubstituted cyclopropyl, or an oxetane substituted by at least one —N(R¹⁸). In some embodiments, L² is —O— or —O—CH₂—; and R¹⁷ is an unsubstituted or substituted hexahydrofuro[3,2-b]furan, an unsubstituted or substituted tetrahydrothiophene-1-oxide, an unsubstituted or substituted 3-oxabicyclo[3.1.0]hexane, an unsubstituted or substituted tetrahydropyran, a disubstituted or trisubstituted cyclopropyl, or an oxetane substituted by at least one —N(R¹⁸). In some embodiments, L² is —O—; and R¹⁷ is an unsubstituted or substituted hexahydrofuro[3,2-b]furan, an unsubstituted or substituted tetrahydrothiophene-1-oxide, an unsubstituted or substituted 3-oxabicyclo[3.1.0]hexane, an unsubstituted or substituted tetrahydropyran, a disubstituted or trisubstituted cyclopropyl, or an oxetane substituted by at least one —N(R¹⁸).

In some embodiments of a compound of Formula (VI), (VIa), (VIb), (VII), (VIIa), (VIIb), (VIIIa), (VIIIb), (VIIIc), or (VIIId), L² is a bond, —CH₂—, —O—, or —O—CH₂—; and R¹⁷ is an unsubstituted or substituted azaspiro[3.3]heptane. In some embodiments, L² is —O— or —O—CH₂—; and R¹⁷ is an unsubstituted or substituted azaspiro[3.3]heptane. In some embodiments, L² is —O—; and R¹⁷ is an unsubstituted or substituted azaspiro[3.3]heptane. In some embodiments, L² is —O—; and R¹⁷ is an unsubstituted azaspiro[3.3]heptane. In some embodiments, L² is —O—; and R¹⁷ is an substituted azaspiro[3.3]heptane, wherein azaspiro[3.3]heptane is substituted with —CH₂CN.

In some embodiments of a compound of Formula (VI), (VIa), (VIb), (VII), (VIIa), (VIIb), (VIIIa), (VIIIb), (VIIIc), or (VIIId), L²-R¹⁷ is

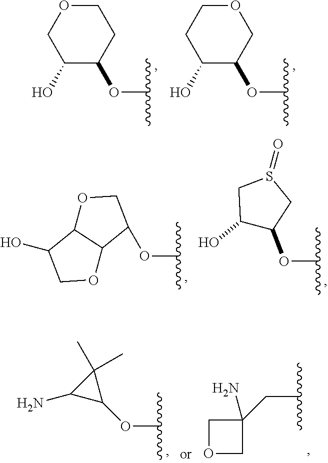

In some embodiments of a compound of Formula (VI), (VIa), (VIb), (VII), (VIIa), (VIIb), (VIIIa), (VIIIb), (VIIIc), or (VIIId), L² is —O—(C₁-C₆ alkylene)-; and R¹⁷ is tetrahydrofuran, where the tetrahydrofuran is unsubstituted or substituted by 1, 2, or 3 R²⁰ groups, wherein: each R²⁰ is independently selected from: halogen, —OH, —NH₂, and —CH₂CN. In some embodiments, L² is —O—(C₁-C₆ alkylene)-; and R¹⁷ is tetrahydrofuran, where the tetrahydrofuran is unsubstituted or substituted by 1, 2, or 3 R²⁰ groups, wherein: each R²⁰ is independently selected from: halogen and —OH. In some embodiments, L² is —O—(C₁-C₆ alkylene)-; and R¹⁷ is tetrahydrofuran, where the tetrahydrofuran is unsubstituted or substituted by 1, 2, or 3 R²⁰ groups, wherein: each R²⁰ is independently selected from: —F, —Cl, and —OH. In some embodiments, L² is —O—(C₁-C₆ alkylene)-; and R¹⁷ is tetrahydrofuran, where the tetrahydrofuran is unsubstituted or substituted by 1, 2, or 3 R²⁰ groups, wherein: each R²⁰ is independently selected from: —F and —OH. In some embodiments, L² is —O—(C₁-C₆ alkylene)-; and R¹⁷ is tetrahydrofuran substituted by 1, 2, or 3 R²⁰ groups, wherein: each R²⁰ is independently selected from: —F and —OH.

In some embodiments of a compound of Formula (VI), (VIa), (VIb), (VII), (VIIa), (VIIb), (VIIIa), (VIIIb), (VIIIc), or (VIIId), L² is —OCH₂—; and R¹⁷ is tetrahydrofuran, where the tetrahydrofuran is unsubstituted or substituted by 1, 2, or 3 R²⁰ groups, wherein: each R²⁰ is independently selected from: halogen, —OH, —NH₂, and —CH₂CN. In some embodiments, L² is —OCH₂—; and R¹⁷ is tetrahydrofuran, where the tetrahydrofuran is unsubstituted or substituted by 1, 2, or 3 R²⁰ groups, wherein: each R²⁰ is independently selected from: halogen and —OH. In some embodiments, L² is —OCH₂—; and R¹⁷ is tetrahydrofuran, where the tetrahydrofuran is unsubstituted or substituted by 1, 2, or 3 R²⁰ groups, wherein: each R²⁰ is independently selected from: —F, —Cl, and —OH. In some embodiments, L² is —OCH₂—; and R¹⁷ is tetrahydrofuran, where the tetrahydrofuran is unsubstituted or substituted by 1, 2, or 3 R²⁰ groups, wherein: each R²⁰ is independently selected from: —F and —OH. In some embodiments, L² is —OCH₂—; and R¹⁷ is tetrahydrofuran substituted by 1, 2, or 3 R²⁰ groups, wherein: each R²⁰ is independently selected from: —F and —OH.

In some embodiments of a compound of Formula (VI), (VIa), (VIb), (VII), (VIIa), (VIIb), (VIIIa), (VIIIb), (VIIIc), or (VIIId), L²-R¹⁷ is

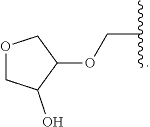 [SMILES: *COC1COCC1O]

In some embodiments of a compound of Formula (VI), (VIa), (VIb), (VII), (VIIa), (VIIb), (VIIIa), (VIIIb), (VIIIc), or (VIIId), L²-R¹⁷ is

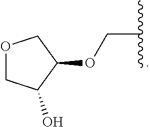 [SMILES: *CO[C@@H]1COC[C@H]1O]

In some embodiments of a compound of Formula (VI), (VIa), (VIb), (VII), (VIIa), (VIIb), (VIIIa), (VIIIb), (VIIIc), or (VIIId), R¹⁷ is a single diastereomer. In some embodiments, R¹⁷ is a single diastereomer and exhibits improved potency to a comparative compound wherein R¹⁷ is a different stereoisomer. In some embodiments, R¹⁷ is a single diastereomer and exhibits improved potency in vitro to a comparative compound wherein R¹⁷ is a different stereoisomer. In some embodiments, R¹⁷ is a single diastereomer and exhibits improved potency in vivo to a comparative compound wherein R¹⁷ is a different stereoisomer.

In some embodiments of a compound of Formula (VI), (VIa), (VIb), (VII), (VIIa), (VIIb), (VIIIa), (VIIIb), (VIIIc), or (VIIId), R¹⁷ is a 4- to 6-membered heterocycloalkyl substituted by 1 or 2 R²⁰ groups, wherein each R²⁰ is independently selected from: halogen, —OH, —OMe, —N(R¹⁸)₂, —NHSO₂R¹⁸, and —CH₂CN. In some embodiments, R¹⁷ is a 4- to 6-membered heterocycloalkyl substituted by 1 or 2 R²⁰ groups, wherein each R²⁰ is independently selected from: halogen, —OH, and —CH₂CN. In some embodiments, R¹⁷ is a 4- to 6-membered heterocycloalkyl substituted by 1 or 2 R²⁰ groups, wherein each R²⁰ is independently selected from: —F, —Cl, —OH, and —CH₂CN. In some embodiments, R¹⁷ is a 4- to 6-membered heterocycloalkyl substituted by —OH.

In some embodiments, R¹⁷ is a 1,2-trans-disubstituted 4- to 6-membered heterocycloalkyl. In some embodiments, L²-R¹⁷ is

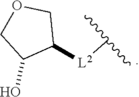

In some embodiments, L²-R¹⁷ is

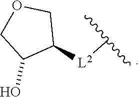

In some embodiments, L²-R¹⁷ is

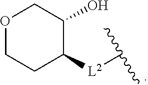

In some embodiments, L²-R¹⁷ is

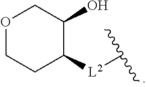

In some embodiments, R¹⁷ is a 1,2-cis-disubstituted 4- to 6-membered heterocycloalkyl. In some embodiments, L²-R¹⁷ is

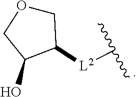

In some embodiments, L²-R¹⁷ is

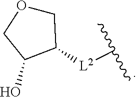

In some embodiments, L²-R¹⁷ is

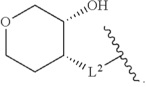

In some embodiments, L²-R¹⁷ is

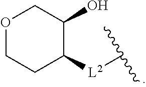

In some embodiments of a compound of Formula (VI), (VIa), (VIb), (VII), (VIIa), (VIIb), (VIIIa), (VIIIb), (VIIIc), or (VIIId), R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂. In some embodiments, R¹³ is —(C₁-C₄ alkylene)-OH, —(C₁-C₄ alkylene)-NH₂, —(C₃-C₆ cycloalkylene)-OH, or —(C₃-C₆ cycloalkylene)-NH₂. In some embodiments, R¹³ is —(C₁-C₄ alkylene)-OH and —(C₃-C₆ cycloalkylene)-OH. In some embodiments, R¹³ is —(C₁-C₄ alkylene)-NH₂ or —(C₃-C₆ cycloalkylene)-OH.

In some embodiments of a compound of Formula (VI), (VIa), (VIb), (VII), (VIIa), (VIIb), (VIIIa), (VIIIb), (VIIIc), or (VIIId), R¹³ is hydrogen, —(C₁-C₄ alkylene)-OH, —(C₁-C₄ alkylene)-NH₂, -(cyclopropylene)-OH, or -(cyclopropylene)-NH₂. In some embodiments, R¹³ is —(C₁-C₄ alkylene)-OH, —(C₁-C₄ alkylene)-NH₂, -(cyclopropylene)-OH, or -(cyclopropylene)-NH₂. In some embodiments, R¹³ is —(C₁-C₄ alkylene)-OH and -(cyclopropylene)-OH. In some embodiments, R¹³ is —(C₁-C₄ alkylene)-NH₂ or -(cyclopropylene)-OH.

In some embodiments of a compound of Formula (VI), (VIa), (VIb), (VII), (VIIa), (VIIb), (VIIIa), (VIIIb), (VIIIc), or (VIIId), R¹³ is hydrogen. In some embodiments, R¹³ is —(C₁-C₄ alkylene)-OH. In some embodiments, R¹³ is —(C₁-C₂ alkylene)-OH.

In some embodiments, R¹³ is —CH₂OH, —CH₂CH₂OH, —CH₂CH₂CH₂OH, —CH₂CH₂CH₂CH₂OH, —CH(CH₃)OH, —CH₂CH(CH₃)OH, —CH(CH₃)CH₂OH, —CH₂CH(CH₂CH₃)OH, or —CH(CH₂CH₃)CH₂OH. In some embodiments, R¹³ is —CH₂OH, —CH₂CH₂OH, or —CH(CH₃)OH. In some embodiments, R¹³ is —CH₂OH or —CH₂CH₂OH. In some embodiments, R¹³ is —CH₂OH. In some embodiments, R¹³ is —CH₂CH₂OH.

In some embodiments of a compound of Formula (VI), (VIa), (VIb), (VII), (VIIa), (VIIb), (VIIIa), (VIIIb), (VIIIc), or (VIIId), R¹³ is —(C₁-C₄ alkylene)-NH₂. In some embodiments, R¹³ is —(C₁-C₂ alkylene)-NH₂. In some embodiments, R¹³ is —CH₂NH₂, —CH₂CH₂NH₂, —CH₂CH₂CH₂NH₂, —CH₂CH₂CH₂CH₂NH₂, —CH(CH₃)NH₂, —CH₂CH(CH₃)NH₂, —CH(CH₃)CH₂NH₂, —CH₂CH(CH₂CH₃)NH₂, or —CH(CH₂CH₃)CH₂NH₂. In some embodiments, R¹³ is —CH₂NH₂, —CH₂CH₂NH₂, or —CH(CH₃)NH₂. In some embodiments, R¹³ is —CH₂NH₂ or —CH₂CH₂NH₂. In some embodiments, R¹³ is —CH₂NH₂.

In some embodiments of a compound of Formula (VI), (VIa), (VIb), (VII), (VIIa), (VIIb), (VIIIa), (VIIIb), (VIIIc), or (VIIId), R¹³ is hydrogen or —(C₁-C₄ alkylene)-OH. In some embodiments, R¹³ is hydrogen or —(C₁-C₂ alkylene)-OH. In some embodiments, R¹³ is hydrogen, —CH₂OH, —CH₂CH₂OH, —CH₂CH₂CH₂OH, —CH₂CH₂CH₂CH₂OH, —CH(CH₃)OH, —CH₂CH(CH₃)OH, —CH(CH₃)CH₂OH, —CH₂CH(CH₂CH₃)OH, or —CH(CH₂CH₃)CH₂OH. In some embodiments, R¹³ is hydrogen, —CH₂OH, —CH₂CH₂OH, or —CH(CH₃)OH. In some embodiments, R¹³ is hydrogen, —CH₂OH or —CH₂CH₂OH. In some embodiments, R¹³ is hydrogen or —CH₂OH.

In some embodiments of a compound of Formula (VI), (VIa), (VIb), (VII), (VIIa), (VIIb), (VIIIa), (VIIIb), (VIIIc), or (VIIId), R¹³ is hydrogen, —(C₁-C₂ alkylene)-OH, or —(C₁-C₂ alkylene)-NH₂. In some embodiments, R¹³ is hydrogen, —CH₂OH, —CH₂CH₂OH, or —CH₂NH₂. In some embodiments, R¹³ is hydrogen, —CH₂OH, or —CH₂NH₂.

In some embodiments of a compound (VI), (VIa), (VIb), (VII), (VIIa), (VIIb), (VIIIa), (VIIIb), (VIIIc), or (VIIId), L² is —X³— or —X⁴—(C₁-C₄ alkylene)-, wherein: X³ and X⁴ are each selected from: —N(H)—, —S—, —S(═O)—, —S(═O)₂—, and —S(═O)(═NH)—.

In some embodiments, the compound is a compound of Formula (VIIIa), or a pharmaceutically acceptable salt, solvate, or stereoisomer thereof. In some embodiments, the compound is a compound of Formula (VIIIb), or a pharmaceutically acceptable salt, solvate, or stereoisomer thereof. In some embodiments, the compound is a compound of Formula (VIIIc), or a pharmaceutically acceptable salt, solvate, or stereoisomer thereof. In some embodiments, the compound is a compound of Formula (VIIId), or a pharmaceutically acceptable salt, solvate, or stereoisomer thereof.

In some embodiments, the compound of Formula (VI) is a compound of Formula (IX):

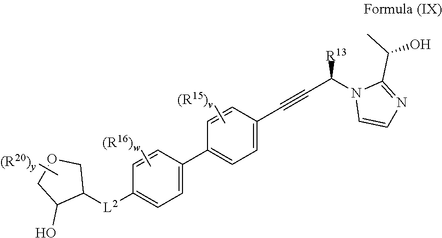

or a pharmaceutically acceptable salt, solvate, or stereoisomer thereof.

In some embodiments of a compound of Formula (IX), R¹³ is hydrogen, —(C₁-C₄ alkylene)-OH, —(C₁-C₄ alkylene)-NH₂, —(C₃-C₆ cycloalkylene)-NH₂; each R¹⁵ and R¹⁶ is —F; each R²⁰ is independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; L² is a bond, —(C₁-C₆ alkylene)-, —X³—, or —X⁴—(C₁-C₆ alkylene)-, wherein: X³ and X⁴ are each independently selected from: —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; w is 0, 1, or 2; y is 0, 1, or 2.

In some embodiments of a compound of Formula (IX), R¹³ is —CH₂—OH, CH₂—NH₂, (cyclopropylene)-OH; or -(cyclopropylene)-NH₂; each R¹⁵ and R¹⁶ is —F; each R²⁰ is independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; L² is a bond, —(C₁-C₆ alkylene)-, —X³—, or —X⁴—(C₁-C₆ alkylene)-, wherein: X³ and X⁴ are each independently selected from: —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; w is 0, 1, or 2; y is 0, 1, or 2.

In some embodiments, the compound of Formula (VI) is a compound of Formula (X):

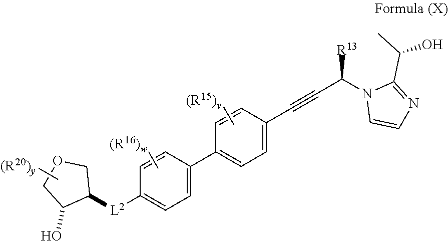

or a pharmaceutically acceptable salt, solvate, or stereoisomer thereof.

In some embodiments of a compound of Formula (IX), R¹³ is hydrogen, —(C₁-C₄ alkylene)-OH, —(C₁-C₄ alkylene)-NH₂, —(C₃-C₆ cycloalkylene)-NH₂; each R¹⁵ and R¹⁶ is —F; each R²⁰ is independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; L² is a bond, —(C₁-C₆ alkylene)-, —X³—, or —X⁴—(C₁-C₆ alkylene)-, wherein: X³ and X⁴ are each independently selected from: —N(R¹⁹)—, —S—, —S(═O)—, —S(═O)₂—, and —S(═O)(═NR¹⁹)—, wherein: R¹⁹ is hydrogen or C₁-C₆ alkyl; v is 0, 1, or 2; w is 0, 1, or 2; y is 0, 1, or 2.

In some embodiments of a compound of Formula (IX), R¹³ is —CH₂—OH, CH₂—NH₂, (cyclopropylene)-OH; or -(cyclopropylene)-NH₂; each R¹⁵ and R¹⁶ is —F; each R²⁰ is independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; L² is a bond, —(C₁-C₆ alkylene)-, —X³—, or —X⁴—(C₁-C₆ alkylene)-, wherein: X³ and X⁴ are each independently selected from: —N(R¹⁹)—, —S—, —S(═O)—, —S(═O)₂—, and —S(═O)(═NR¹⁹)—, wherein: R¹⁹ is hydrogen or C₁-C₆ alkyl; v is 0; w is 0; y is 0, 1, or 2.

In some embodiments of a compound of Formula (VI), (VIa), (VIb), (VII), (VIIa), (VIIb), (VIIIa), (VIIIb), (VIIIc), (VIIId), (IX), or (X), —(C₃-C₆ cycloalkylene)-OH or —(C₃-C₆ cycloalkylene)-NH₂. In some embodiments, R¹³ is —(C₃-C₅ cycloalkylene)-OH or —(C₃-C₅ cycloalkylene)-NH₂. In some embodiments, R¹³ is —(C₃-C₄ cycloalkylene)-OH or —(C₃-C₄ cycloalkylene)-NH₂. In some embodiments, R¹³ is -(cyclopropylene)-OH or -(cyclopropylene)-NH₂. In some embodiments, R¹³ is

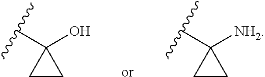 [SMILES: *C1(O)CC1 ; "or" ; *C1(N)CC1]

In some embodiments of a compound of Formula (VI), (VIa), (VIb), (VII), (VIIa), (VIIb), (VIIIa), (VIIIb), (VIIIc), (VIIId), (IX), or (X), R¹³ is C₃-C₆ cycloalkylene)-OH. In some embodiments, —(C₃-C₅ cycloalkylene)-OH. In some embodiments, R¹³ is —(C₃-C₄ cycloalkylene)-OH. In some embodiments, R¹³ is -(cyclopropylene)-OH. In some embodiments, R¹³ is

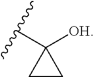 [SMILES: *C1(O)CC1]

In some embodiments of a compound of Formula (VI), (VIa), (VIb), (VII), (VIIa), (VIIb), (VIIIa), (VIIIb), (VIIIc), (VIIId), (IX), or (X), R¹³ is —(C₃-C₆ cycloalkylene)-NH₂. In some embodiments, R¹³ is —(C₃-C₅ cycloalkylene)-NH₂. In some embodiments, R¹³ is —(C₃-C₄ cycloalkylene)-NH₂. In some embodiments, R¹³ is -(cyclopropylene)-NH₂. In some embodiments, R¹³ is

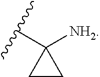 [SMILES: *C1(N)CC1]

In some embodiments of a compound of Formula (VI), (VIa), (VIb), (VII), (VIIa), (VIIb), (VIIIa), (VIIIb), (VIIIc), (VIIId), (IX), or (X) L² is —X³— or —X⁴—(C₁-C₄ alkylene)-, wherein: X³ and X⁴ are each selected from: —N(H)—, —S—, —S(═O)—, —S(═O)₂—, and —S(═O)(═NH)—. In some embodiments, L² is —X³— or —X⁴—(C₁-C₄ alkylene)-, wherein: X³ and X⁴ are each selected from: —N(H)—, —S—, —S(═O)—, and —S(═O)₂—. In some embodiments, L² is —X³— or —X⁴—(C₁-C₄ alkylene)-, wherein: X³ and X⁴ are each selected from: —N(H)—, —S—, —S(═O)—, and —S(═O)(═NH)—. In some embodiments, L² is —X³— or —X⁴—(C₁-C₄ alkylene)-, wherein: X³ and X⁴ are each selected from: —N(H)—, —S—, —S(═O)₂—, and —S(═O)(═NH)—. In some embodiments, L² is —X³— or —X⁴—(C₁-C₄ alkylene)-, wherein: X³ and X⁴ are each selected from: —N(H)— and —S—.

In some embodiments of a compound of Formula (VI), (VIa), (VIb), (VII), (VIIa), (VIIb), (VIIIa), (VIIIb), (VIIIc), (VIIId), (IX), or (X), L² is —N(R¹⁹)— or —N(R¹⁹)—(C₁-C₆ alkylene)-. In some embodiments, L² is —S— or —S—(C₁-C₆ alkylene)-. In some embodiments, L² is —S(═O)— or —S(═O)—(C₁-C₆ alkylene)-. In some embodiments, L² is —S(═O)₂— or —S(═O)₂—(C₁-C₆ alkylene)-. In some embodiments, L² is —S(═O)(═NR¹⁹)— or —S(═O)(═NR¹⁹)—(C₁-C₆ alkylene)-. In some embodiments, L² is —N(R¹⁹)—. In some embodiments, R¹⁹ is hydrogen. In some embodiments, L² is —S—. In some embodiments, L² is —S(═O)—. In some embodiments, L² is —S(═O)₂—. In some embodiments, L² is —S(═O)(═NR¹⁹). In some embodiments, L² is —N(R¹⁹)—(C₁-C₆ alkylene)-. In some embodiments, L² is —S—(C₁-C₆ alkylene)-. In some embodiments, L² is —S(═O)—(C₁-C₆ alkylene)-. In some embodiments, L² is —S(═O)₂—(C₁-C₆ alkylene)-. In some embodiments, L² is —S(═O)(═NR¹⁹)—(C₁-C₆ alkylene)-. In some embodiments, L² is —N(H)— or —N(H)—(CH₂)—. In some embodiments, L² is —S— or —S—(CH₂)—. In some embodiments, L² is —S(═O)— or —S(═O)—(CH₂)—. In some embodiments, L² is —S(═O)₂— or —S(═O)₂—(CH₂)—. In some embodiments, L² is —S(═O)(═NH)— or —S(═O)(═NH)—(CH₂)—. In some embodiments, L² is —N(H)—(CH₂)—. In some embodiments, L² is —S—(CH₂)—. In some embodiments, L² is —S(═O)—(CH₂)—. In some embodiments, L² is —S(═O)₂—(CH₂)—. In some embodiments, L² is —S(═O)(═NH)—(CH₂)—.

In some embodiments of a compound of Formula (VI), (VIa), (VIb), (VII), (VIIa), (VIIb), (VIIIa), (VIIIb), (VIIIc), (VIIId), (IX), or (X), v is 0. In some embodiments, v is 1. In some embodiments, v is 2. In some embodiments, v is 1 or 2.

In some embodiments of a compound of Formula (VI), (VIa), (VIb), (VII), (VIIa), (VIIb), (VIIIa), (VIIIb), (VIIIc), (VIIId), (IX), or (X), w is 0. In some embodiments, w is 1. In some embodiments, w is 2. In some embodiments, w is 1 or 2.

In some embodiments of a compound of Formula (VI), (VIa), (VIb), (VII), (VIIa), (VIIb), (VIIIa), (VIIIb), (VIIIc), (VIIId), (IX), or (X), w is 0; and v is 0.

In some embodiments of a compound of Formula (IX) or (X), y is 0. In some embodiments, y is 1. In some embodiments, y is 2. In some embodiments, y is 1 or 2.

In some embodiments of a compound, salt, solvate, or stereoisomer of Formula (IX) or (X), each R²⁰ is independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein: each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃; or two R¹⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo.

In some embodiments of a compound, salt, solvate, or stereoisomer of Formula (IX) or (X), each R²⁰ is independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN and C₁-C₄ alkyl, further wherein: each R¹⁸ is independently hydrogen or C₁-C₄ alkyl. In some embodiments, each R²⁰ is independently selected from: halogen, —OR¹⁸, —N(R¹⁸)₂, —CO₂R¹⁸, —CON(R¹⁸)₂, —CH₂CN, and C₁-C₄ alkyl, further wherein: each R¹⁸ is independently hydrogen or C₁-C₄ alkyl. In some embodiments, each R²⁰ is independently selected from: halogen, —OR¹⁸, —N(R¹⁸)₂, —CH₂CN, and C₁-C₄ alkyl, further wherein: each R¹⁸ is independently hydrogen or C₁-C₄ alkyl. In some embodiments, each R²⁰ is independently selected from: halogen, —OH, —NH₂, —CO₂H, —CONH₂, —CH₂CN, and C₁-C₄ alkyl. In some embodiments, each R²⁰ is independently selected from: halogen, —OH, —NH₂, —CH₂CN, and C₁-C₄ alkyl. In some embodiments, each R²⁰ is independently selected from: halogen, —OH, —NH₂, and C₁-C₄ alkyl. In some embodiments, each R²⁰ is independently selected from: —F, —Cl, —OH, —NH₂, and C₁-C₄ alkyl. In some embodiments, each R²⁰ is independently selected from: —F, —Cl, —OH, —NH₂, and methyl. In some embodiments, each R²⁰ is independently selected from: —F, —Cl, —OH, and —NH₂. In some embodiments, each R²⁰ is independently selected from: —F, —OH, and —NH₂. In some embodiments, each R²⁰ is independently selected from: —F and —OH. In some embodiments, each R²⁰ is —OH.

In some embodiments, the compound is selected from:

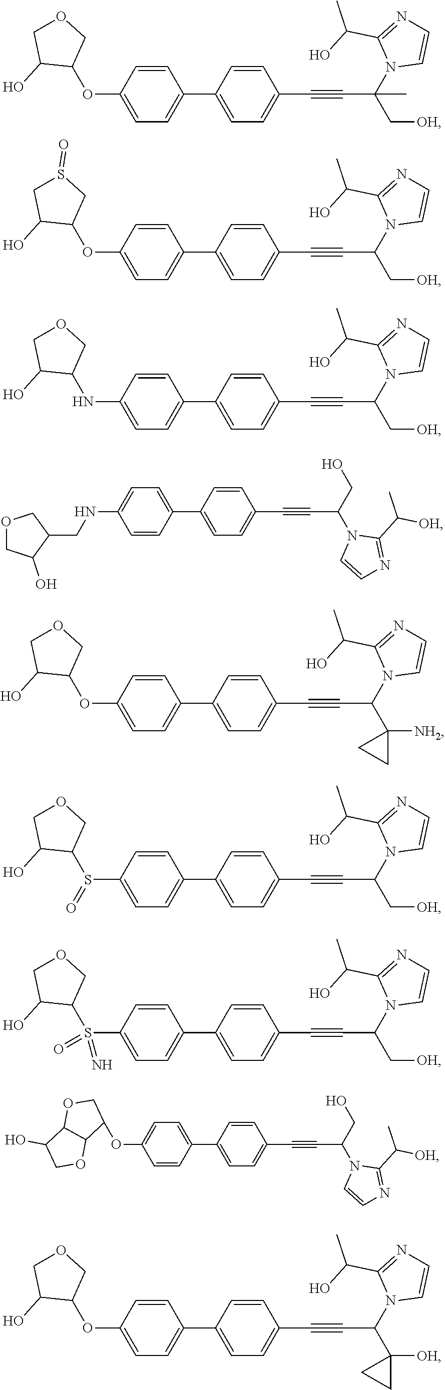

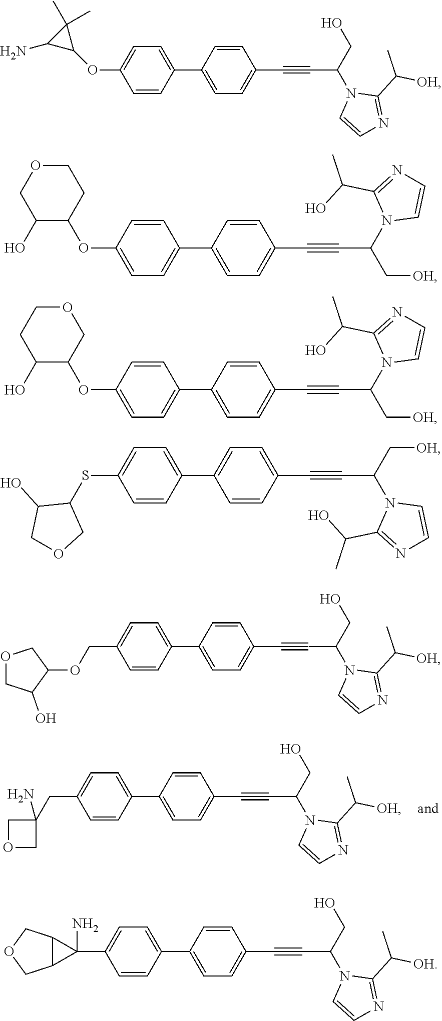

In some embodiments, the compound is a compound of Table 2, or a pharmaceutically acceptable salt, solvate, or stereoisomer thereof. In some embodiments, the compound is a diastereomer of a compound of Table 2, or a pharmaceutically acceptable salt, solvate, or stereoisomer thereof.

TABLE 2

| Cmpd No | Structure | IUPAC Name |
|---|---|---|
| 27 | 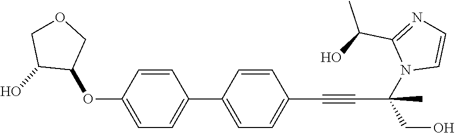 [SMILES: C[C@H](O)c1nccn1[C@@](C)(C#Cc1ccc(-c2ccc(O[C@@H]3COC[C@H]3O)cc2)cc1)CO] | (3R,4R)-4-((4′-((S)-4-hydroxy-3- (2-((S)-1-hydroxyethyl)-1H- imidazol-1-yl)-3-methylbut-1-yn- 1-yl)-[1,1′-biphenyl]-4-yl)oxy) tetrahydrofuran-3-ol |
| 28** | 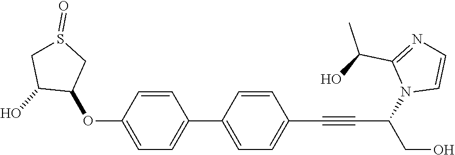 [SMILES: C[C@H](O)c1nccn1[C@@H](C#Cc1ccc(-c2ccc(O[C@@H]3CS(=O)C[C@H]3O)cc2)cc1)CO] | (3S,4S)-3-hydroxy-4-((4′-((S)-4- hydroxy-3-(2-((S)-1-hydroxyethyl)-1H- imidazol-1-yl)but-1-yn-1-yl)-[1,1′- biphenyl]-4-yl)oxy)tetrahydrothiophene 1-oxide |
| | (Isomer 1) | |
| 29** | 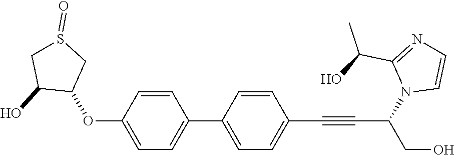 [SMILES: C[C@H](O)c1nccn1[C@@H](C#Cc1ccc(-c2ccc(O[C@H]3CS(=O)C[C@@H]3O)cc2)cc1)CO] | (3R,4R)-3-hydroxy-4-((4′-((S)-4- hydroxy-3-(2-((S)-1- hydroxyethyl)-1H-imidazol-1- yl)but-1-yn-1-yl)-[1,1′-biphenyl]- 4-yl)oxy)tetrahydrothiophene 1- oxide |
| | (Isomer 2) | |
| 30 | 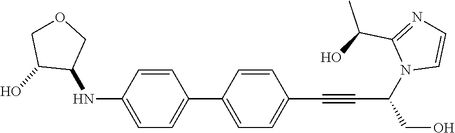 [SMILES: C[C@H](O)c1nccn1[C@@H](C#Cc1ccc(-c2ccc(N[C@@H]3COC[C@H]3O)cc2)cc1)CO] | (3S,4R)-4-((4′-((S)-4-hydroxy-3- (2-((S)-1-hydroxyethyl)-1H- imidazol-1-yl)but-1-yn-1-yl)- [1,1′-biphenyl]-4- yl)amino)tetrahydrofuran-3-ol |
| 31 | 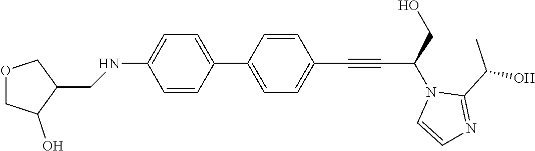 [SMILES: C[C@H](O)c1nccn1[C@@H](C#Cc1ccc(-c2ccc(NCC3COCC3O)cc2)cc1)CO] | 4-(((4′-((S)-4-hydroxy-3-(2-((S)- 1-hydroxyethyl)-1H-imidazol-1- yl)but-1-yn-1-yl)-[1,1′-biphenyl]- 4-yl)amino)methyl)tetrahydrofuran- 3-ol |
| 32 | 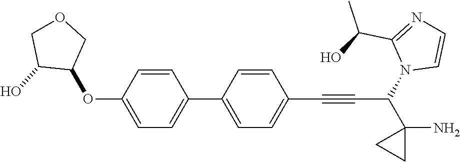 | (3R,4R)-4-((4′-((S)-3-(1- aminocyclopropyl)-3-(2-((S)-1- hydroxyethyl)-1H-imidazol-1- yl)prop-1-yn-1-yl)-[1,1′- biphenyl]-4- yl)oxy)tetrahydrofuran-3-ol |
| 33 | 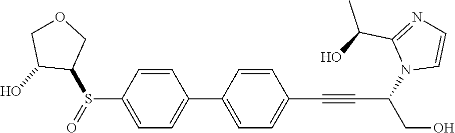 [SMILES: C[C@H](O)c1nccn1[C@@H](C#Cc1ccc(-c2ccc([S@@](=O)C3COC[C@H]3O)cc2)cc1)CO] | (3R,4R)-4-((S)-(4′-((S)-4- hydroxy-3-(2-((S)-1- hydroxyethyl)-1H-imidazol-1- yl)but-1-yn-1-yl)-[1,1′-biphenyl]- 4-yl)sulfinyl)tetrahydrofuran-3-ol |
| 34 | 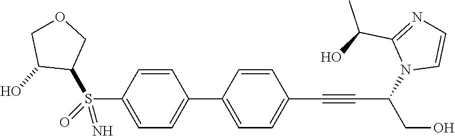 [SMILES: C[C@H](O)c1nccn1[C@@H](C#Cc1ccc(-c2ccc([S@](=N)(=O)C3COC[C@H]3O)cc2)cc1)CO] | (4′-((S)-4-hydroxy-3-(2-((S)-1- hydroxyethyl)-1H-imidazol-1- yl)but-1-yn-1-yl)-[1,1′-biphenyl]- 4-yl)((3R,4R)-4- hydroxytetrahydrofuran-3- yl)(imino)-l6-sulfanone |
| 35 | 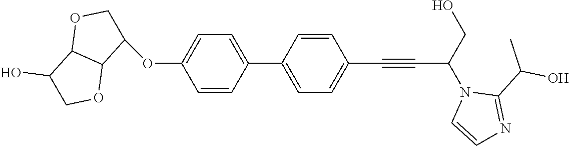 [SMILES: CC(O)c1nccn1C(C#Cc1ccc(-c2ccc(OC3COC4C(O)COC34)cc2)cc1)CO] | 6-((4′-(4-hydroxy-3-(2-(1- hydroxyethyl)-1H-imidazol-1- yl)but-1-yn-1-yl)-[1,1′-biphenyl]- 4-yl)oxy)hexahydrofuro[3,2- b]furan-3-ol |
| 36 | 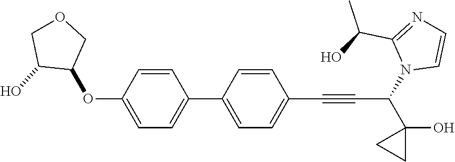 | (3R,4R)-4-((4′-((S)-3-(1- hydroxycyclopropyl)-3-(2-((S)-1- hydroxyethyl)-1H-imidazol-1- yl)prop-1-yn-1-yl)-[1,1′-biphenyl]- 4-yl)oxy)tetrahydrofuran-3-ol |
| 37 | 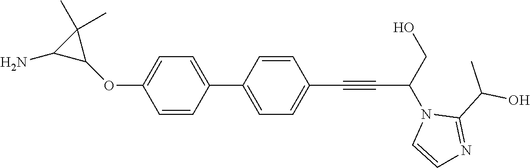 [SMILES: CC(O)c1nccn1C(C#Cc1ccc(-c2ccc(OC3C(N)C3(C)C)cc2)cc1)CO] | 4-(4′-(3-amino-2,2- dimethylcyclopropoxy)- [1,1′-biphenyl]-4-yl)-2-(2-(1- hydroxyethyl)-1H-imidazol-1- yl)but-3-yn-1-ol |
| 38 | 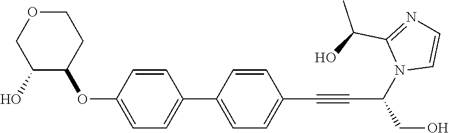 | (3R,4R)-4-((4′-((S)-4-hydroxy-3- (2-((S)-1-hydroxyethyl)-1H- imidazol-1-yl)but-1-yn-1-yl)- [1,1′-biphenyl]-4- yl)oxy)tetrahydro-2H-pyran-3-ol |
| 39 | 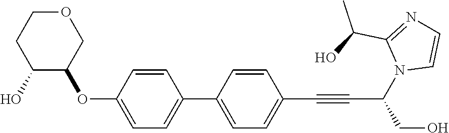 | (3R,4R)-3-((4′-((S)-4-hydroxy-3- (2-((S)-1-hydroxyethyl)-1H- imidazol-1-yl)but-1-yn-1-yl)- [1,1′-biphenyl]-4- yl)oxy)tetrahydro-2H-pyran-ol |
| 40 | 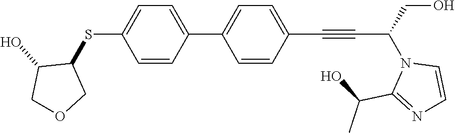 | (3S,4S)-4-((4′-((R)-4-hydroxy-3- (2-((R)-1-hydroxyethyl)-1H- imidazol-1-yl)but-1-yn-1-yl)- [1,1′-biphenyl]-4- yl)thio)tetrahydrofuran-3-ol |
| 41 | 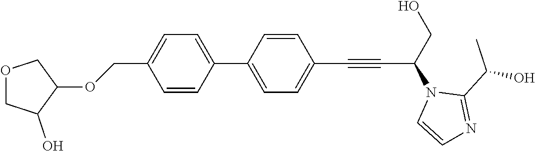 [SMILES: C[C@H](O)c1nccn1[C@@H](C#Cc1ccc(-c2ccc(COC3COCC3O)cc2)cc1)CO] | 4-((4′-((S)-4-hydroxy-3-(2-((S)-1- hydroxyethyl)-1H-imidazol-1- yl)but-1-yn-1-yl)-[1,1′-biphenyl]- 4-yl)methoxy)tetrahydrofuran-3-ol |
| 42 | 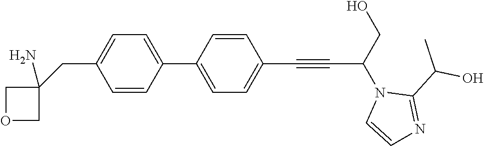 [SMILES: CC(O)c1nccn1C(C#Cc1ccc(-c2ccc(CC3(N)COC3)cc2)cc1)CO] | 4-(4′-((3-aminooxetan-3- yl)methyl)-[1,1′-biphenyl]-4-yl)- 2-(2-(1-hydroxyethyl)-1H- imidazol-1-yl)but-3-yn-1-ol |
| 43 | 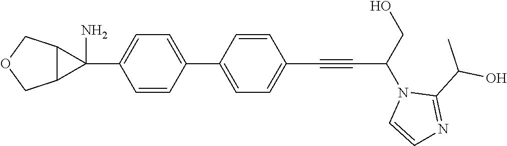 [SMILES: CC(O)c1nccn1C(C#Cc1ccc(-c2ccc(C3(N)C4COCC43)cc2)cc1)CO] | 4-(4′-(6-amino-3- oxabicyclo[3.1.0]hexan-6-yl)- [1,1′-biphenyl]-4-yl)-2-(2-(1- hydroxyethyl)-1H-imidazol-1- yl)but-3-yn-1-ol |
| 65 | 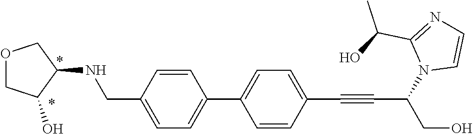 [SMILES: C[C@H](O)c1nccn1[C@@H](C#Cc1ccc(-c2ccc(CN[C@@H]3COC[C@H]3O)cc2)cc1)CO] | (rac-trans)-4-(((4′-((S)-4-hydroxy- 3-(2-((S)-1-hydroxyethyl)-1H- imidazol-1-yl)but-1-yn-1-yl)- [1,1′-biphenyl]-4- yl)amino)methyl)tetrahydrofuran- 3-ol |
| | *racemic-trans | |
| 66 | 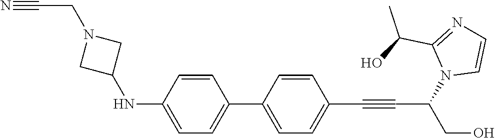 [SMILES: C[C@H](O)c1nccn1[C@@H](C#Cc1ccc(-c2ccc(NC3CN(CC#N)C3)cc2)cc1)CO] | 2-(3-((4′-((S)-4-hydroxy-3-(2- ((S)-1-hydroxyethyl)-1H- imidazol-1-yl)but-1-yn-1-yl)- [1,1′-biphenyl]-4- yl)amino)azetidin-1-yl)acetonitrile |
| 67 | 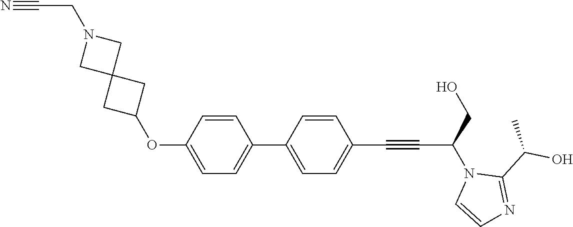 [SMILES: C[C@H](O)c1nccn1[C@@H](C#Cc1ccc(-c2ccc(OC3CC4(C3)CN(CC#N)C4)cc2)cc1)CO] | 2-(6-((4′-((S)-4-hydroxy-3-(2- ((S)-1-hydroxyethyl)-1H- imidazol-1-yl)but-1-yn-1-yl)- [1,1′-biphenyl]-4-yl)oxy)-2- azaspiro[3.3]heptan-2- yl)acetonitrile |
| 68 | 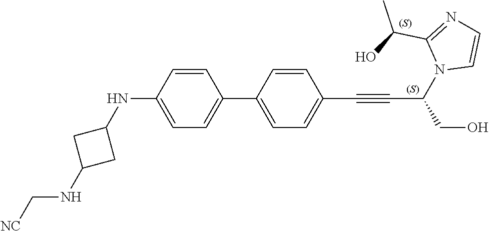 | 2-((3-((4′-((S)-4-hydroxy-3-(2- ((S)-1-hydroxyethyl)-1H- imidazol-1-yl)but-1-yn-1-yl)- [1,1′-biphenyl]-4- yl)amino)cyclobutyl)amino) acetonitrile |
| 69 | 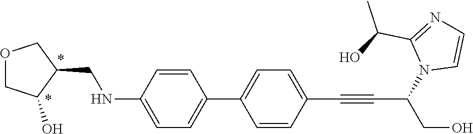 [SMILES: C[C@H](O)c1nccn1[C@@H](C#Cc1ccc(-c2ccc(NC[C@@H]3COC[C@H]3O)cc2)cc1)CO] | (rac-trans)-4-(((4′-((S)-4- hydroxy-3-(2-((S)-1- hydroxyethyl)-1H-imidazol-1- yl)but-1-yn-1-yl)-[1,1′-biphenyl]- 4-yl)amino)methyl)tetrahydrofuran- 3-ol |
| | *racemic-trans | |
| 70 | 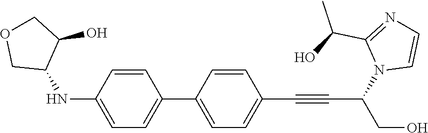 [SMILES: C[C@H](O)c1nccn1[C@@H](C#Cc1ccc(-c2ccc(N[C@@H]3COC[C@H]3O)cc2)cc1)CO] | (3S,4R)-4-((4′-((S)-4-hydroxy-3- (2-((S)-1-hydroxyethyl)-1H- imidazol-1-yl)but-1-yn-1-yl)- [1,1′-biphenyl]-4- yl)amino)tetrahydrofuran-3-ol |
| 71 | 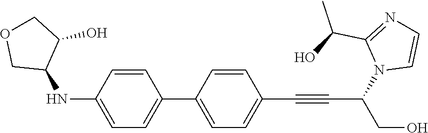 [SMILES: C[C@H](O)c1nccn1[C@@H](C#Cc1ccc(-c2ccc(N[C@H]3COC[C@@H]3O)cc2)cc1)CO] | (3R,4S)-4-((4′-((S)-4-hydroxy-3- (2-((S)-1-hydroxyethyl)-1H- imidazol-1-yl)but-1-yn-1-yl)- [1,1′-biphenyl]-4- yl)amino)tetrahydrofuran-3-ol |
| 72** | 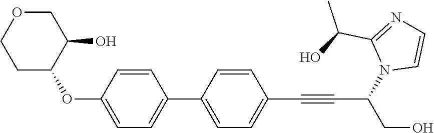 [SMILES: C[C@H](O)c1nccn1[C@@H](C#Cc1ccc(-c2ccc(O[C@@H]3CCOC[C@H]3O)cc2)cc1)CO] | (3R,4R)-4-((4′-((S)-4-hydroxy-3- (2-((S)-1-hydroxyethyl)-1H- imidazol-1-yl)but-1-yn-1-yl)- [1,1′-biphenyl]-4- yl)oxy)tetrahydro-2H-pyran-3-ol |
| 73** | 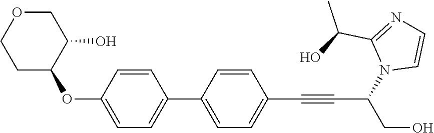 [SMILES: C[C@H](O)c1nccn1[C@@H](C#Cc1ccc(-c2ccc(O[C@H]3CCOC[C@@H]3O)cc2)cc1)CO] | (3S,4S)-4-((4′-((S)-4-hydroxy-3- (2-((S)-1-hydroxyethyl)-1H- imidazol-1-yl)but-1-yn-1-yl)- [1,1′-biphenyl]-4- yl)oxy)tetrahydro-2H-pyran-3-ol |
| 74 | 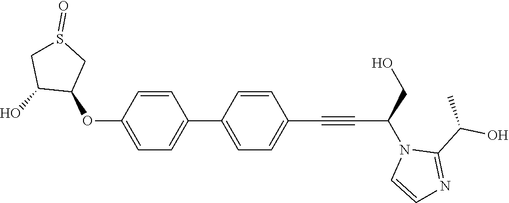 [SMILES: C[C@H](O)c1nccn1[C@@H](C#Cc1ccc(-c2ccc(O[C@@H]3CS(=O)C[C@H]3O)cc2)cc1)CO] | (3S,4S)-3-hydroxy-4-((4′-((S)-4- hydroxy-3-(2-((S)-1- hydroxyethyl)-1H-imidazol-1- yl)but-1-yn-1-yl)-[1,1′-biphenyl]- 4-yl)oxy)tetrahydrothiophene 1- oxide |
| 75 | 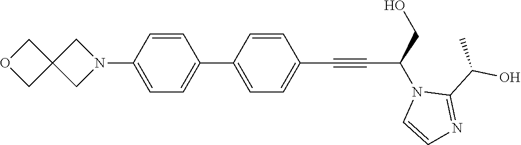 [SMILES: C[C@H](O)c1nccn1[C@@H](C#Cc1ccc(-c2ccc(N3CC4(COC4)C3)cc2)cc1)CO] | (S)-4-(4′-(2-oxa-6- azaspiro[3.3]heptan-6-yl)- [1,1′-biphenyl]-4-yl)-2-(2-((S)-1- hydroxyethyl)-1H-imidazol-1- yl)but-3-yn-1-ol |
| 76 | 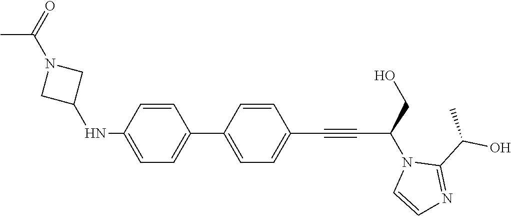 [SMILES: CC(=O)N1CC(Nc2ccc(-c3ccc(C#C[C@@H](CO)n4ccnc4[C@H](C)O)cc3)cc2)C1] | 1-(3-((4′-((S)-4-hydroxy-3-(2- ((S)-1-hydroxyethyl)-1H- imidazol-1-yl)but-1-yn-1-yl)- [1,1′-biphenyl]-4- yl)amino)azetidin-1-yl)ethan-1- one |
| 77** | 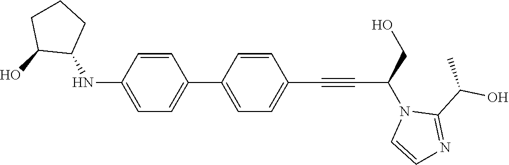 [SMILES: C[C@H](O)c1nccn1[C@@H](C#Cc1ccc(-c2ccc(N[C@H]3CCC[C@@H]3O)cc2)cc1)CO] | (1S,2S)-2-((4′-((S)-4-hydroxy-3- (2-((S)-1-hydroxyethyl)-1H- imidazol-1-yl)but-1-yn-1-yl)- [1,1′-biphenyl]-4- yl)amino)cyclopentan-1-ol |
| 78** | 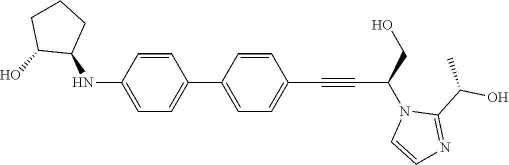 [SMILES: C[C@H](O)c1nccn1[C@@H](C#Cc1ccc(-c2ccc(N[C@@H]3CCC[C@H]3O)cc2)cc1)CO] | (1R,2R)-2-((4′-((S)-4-hydroxy-3- (2-((S)-1-hydroxyethyl)-1H- imidazol-1-yl)but-1-yn-1-yl)- [1,1′-biphenyl]-4- yl)amino)cyclopentan-1-ol |
| 79 | 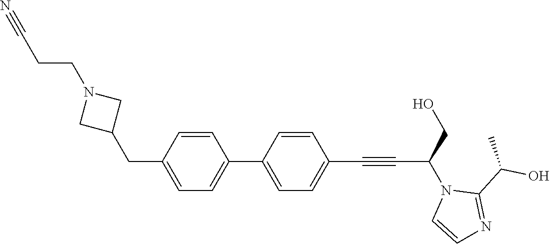 [SMILES: C[C@H](O)c1nccn1[C@@H](C#Cc1ccc(-c2ccc(CC3CN(CCC#N)C3)cc2)cc1)CO] | 3-(3-((4′-((S)-4-hydroxy-3-(2- ((S)-1-hydroxyethyl)-1H- imidazol-1-yl)but-1-yn-1-yl)- [1,1′-biphenyl]-4- yl)amino)azetidin-1- yl)propanenitrile |
| 80 | 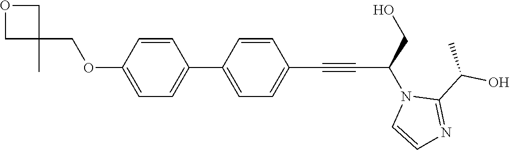 [SMILES: C[C@H](O)c1nccn1[C@@H](C#Cc1ccc(-c2ccc(OCC3(C)COC3)cc2)cc1)CO] | (S)-2-(2-((S)-1-hydroxyethyl)-1H- imidazol-1-yl)-4-(4′-((3- methyloxetan-3-yl)methoxy)- [1,1′-biphenyl]-4-yl)but-3-yn-1-ol |
| 81 | 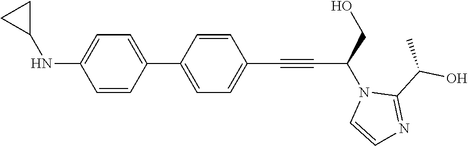 [SMILES: C[C@H](O)c1nccn1[C@@H](C#Cc1ccc(-c2ccc(NC3CC3)cc2)cc1)CO] | (S)-4-(4′-(cyclopropylamino)- [1,1′-biphenyl]-4-yl)-2-(2-((S)-1- hydroxyethyl)-1H-imidazol-1- yl)but-3-yn-1-ol |

**stereochemistry arbitrarily assigned. After chiral separation, single stereoisomers are isolated but the absolute configuration of the stereochemical
center is unknown.

In some embodiments, the compound is a compound of Formula (XI):

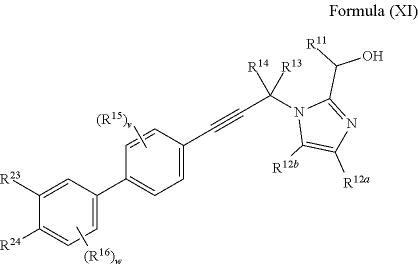

or a pharmaceutically acceptable salt, solvate, or stereoisomer thereof, wherein:
   R¹¹ is C₁-C₄ alkyl;
   R^{12a} and R^{12b} are each independently hydrogen, halogen, or C₁-C₄ alkyl;
   R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂;
   R¹⁴ is hydrogen or C₁-C₄ alkyl;
   each R¹⁵ and R¹⁶ is independently halogen, or C₁-C₄ alkyl;
   R²³ and R²⁴ are taken together with the intervening carbon atoms connecting R²³ to R²⁴ to form a Ring A that is C₃-C₆ cycloalkyl or 4- to 8-membered heterocycloalkyl, wherein the C₃-C₆ cycloalkyl or 4- to 8-membered heterocycloalkyl is unsubstituted or substituted by 1, 2, or 3 groups independently selected from: halogen, —OR²⁵, —N(R²⁵)₂, —CO₂R²⁵, —COR²⁵, —CON(R²⁵)₂, C₁-C₄ alkyl, C₁-C₄ hydroxyalkyl, C₁-C₄ aminoalkyl, and oxo; wherein:
   each R²⁵ is independently hydrogen or C₁-C₄ alkyl, wherein the alkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, and oxo; or two R²⁵ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo;
   v is 0, 1, or 2; and
w is 0, 1, or 2. In some embodiments of a compound of Formula (XI), R¹¹ is unsubstituted C₁-C₄ alkyl. In some embodiments, R¹¹ is C₁-C₂ alkyl. In some embodiments, R¹¹ is —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃. In some embodiments, R¹¹ is —CH₃ or —CH₂CH₃. In some embodiments, R¹¹ is —CH₃.

In some embodiments of a compound of Formula (XI), R^{12a} and R^{12b} are each independently R^{12a} and R^{12b} are each independently hydrogen, halogen, or unsubstituted C₁-C₄ alkyl. In some embodiments, R^{12a} and R^{12b} are each independently hydrogen, —F, —Cl, —Br, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, R^{12a} and R^{12b} are each independently hydrogen, —F, —Cl, —CH₃, —CH₂CH₃, or —CH(CH₃)₂.

In some embodiments of a compound of Formula (XI), R^{12a} is hydrogen. In some embodiments, R^{12b} is hydrogen. In some embodiments, R^{12a} and R^{12b} are each hydrogen.

In some embodiments of a compound of Formula (XI), R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂. In some embodiments, R¹³ is hydrogen. In some embodiments, R¹³ is —(C₁-C₄ alkylene)-OH. In some embodiments, R¹³ is —(C₁-C₂ alkylene)-OH.

In some embodiments of a compound of Formula (XI), R¹³ is —CH₂OH, —CH₂CH₂OH, —CH₂CH₂CH₂OH, —CH₂CH₂CH₂CH₂OH, —CH(CH₃)OH, —CH₂CH(CH₃)OH, —CH(CH₃)CH₂OH, —CH₂CH(CH₂CH₃)OH, or —CH(CH₂CH₃)CH₂OH. In some embodiments, R¹³ is —CH₂OH, —CH₂CH₂OH, or —CH(CH₃)OH. In some embodiments, R¹³ is —CH₂OH or —CH₂CH₂OH. In some embodiments, R¹³ is —CH₂OH.

In some embodiments of a compound of Formula (XI), R¹⁴ is hydrogen or unsubstituted C₁-C₄ alkyl. In some embodiments, R¹⁴ is hydrogen or C₁-C₂ alkyl. In some embodiments, R¹⁴ is hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃. In some embodiments, R¹⁴ is hydrogen, —CH₃ or —CH₂CH₃. In some embodiments of a compound of Formula (XI), R¹⁴ is hydrogen or —CH₃. In some embodiments, R¹⁴ is hydrogen. In some embodiments, R¹⁴ is —CH₃.

In some embodiments of a compound of Formula (XI), w is 0 and v is 0. In some embodiments, w is 0. In some embodiments, v is 0.

In some embodiments of a compound of Formula (XI), R²³ and R²⁴ are taken together with the intervening carbon atoms connecting R²³ to R²⁴ to form a Ring A that is C₃-C₆ cycloalkyl or 4- to 8-membered heterocycloalkyl, wherein C₃-C₆ cycloalkyl or 4- to 8-membered heterocycloalkyl is unsubstituted or substituted by 1, 2, or 3 groups independently selected from: halogen, C₁-C₄ alkyl, C₁-C₄ hydroxyalkyl, C₁-C₄ aminoalkyl, and oxo.

In some embodiments of a compound of Formula (XI), R²³ and R²⁴ are taken together with the intervening carbon atoms connecting R²³ to R²⁴ to form a Ring A is 4- to 8-membered heterocycloalkyl, wherein 4- to 8-membered heterocycloalkyl is unsubstituted or substituted with 1, 2, or 3 groups independently selected from halogen, C₁-C₄ alkyl, and oxo. In some embodiments, R²³ and R²⁴ are taken together with the intervening carbon atoms connecting R²³ to R²⁴ to form a Ring A is 4- to 6-membered heterocycloalkyl containing 1-2 O atoms or 1-2 N atoms, wherein 4- to 8-membered heterocycloalkyl is unsubstituted or substituted with 1, 2, or 3 groups independently selected from halogen, C₁-C₄ alkyl, and oxo.

In some embodiments of a compound of Formula (XI), R²³ and R²⁴ are taken together with the intervening carbon atoms connecting R²³ to R²⁴ to form a Ring A that is 4- to 8-membered heterocycloalkyl, wherein the 4- to 8-membered heterocycloalkyl is unsubstituted or substituted by 1, 2, or 3 groups independently selected from: halogen, —OR²⁵, —N(R²⁵)₂, —CO₂R²⁵, —COR²⁵, —CON(R²⁵)₂, C₁-C₄ alkyl, C₁-C₄ hydroxyalkyl, C₁-C₄ aminoalkyl, and oxo.

In some embodiments of a compound of Formula (XI), R²³ and R²⁴ are taken together with the intervening carbon atoms connecting R²³ to R²⁴ to form a Ring A that is 5- to 6-membered heterocycloalkyl, wherein the 5- to 6-membered heterocycloalkyl is unsubstituted or substituted by 1, 2, or 3 groups independently selected from: halogen, C₁-C₄ alkyl, C₁-C₄ hydroxyalkyl, and oxo.

In some embodiments of a compound of Formula (XI), R²³ and R²⁴ are taken together with the intervening carbon atoms connecting R²³ to R²⁴ to form a Ring A that is

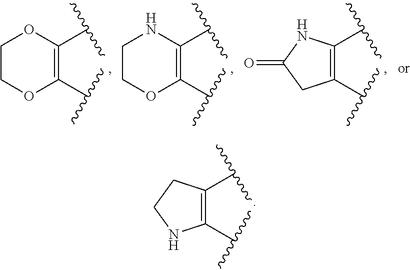

In some embodiments of a compound of Formula (XI),

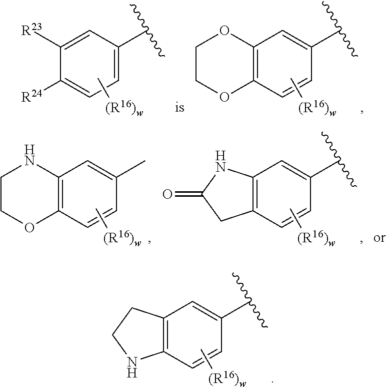

In some embodiments, the compound is a compound of Table 3, or a pharmaceutically acceptable salt, solvate, or stereoisomer thereof. In some embodiments, the compound is a diastereomer of a compound of Table 3, or a pharmaceutically acceptable salt, solvate, or stereoisomer thereof.

TABLE 3

| Cmpd | | |
|---|---|---|
| No | Structure | IUPAC Name |
| 82 | 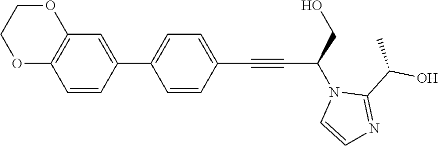 [SMILES: C[C@H](O)c1nccn1[C@@H](C#Cc1ccc(-c2ccc3c(c2)OCCO3)cc1)CO] | (S)-4-(4-(2,3- dihydrobenzo[b][1,4]dioxin-6- yl)phenyl)-2-(2-((S)-1- hydroxyethyl)-1H-imidazol-1-yl) but-3-yn-1-ol |
| 83 | 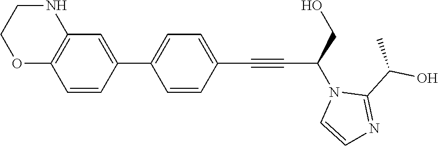 [SMILES: C[C@H](O)c1nccn1[C@@H](C#Cc1ccc(-c2ccc3c(c2)NCCO3)cc1)CO] | (S)-4-(4-(3,4-dihydro-2H- benzo[b][1,4]oxazin-6-yl)phenyl)-2- (2-((S)-1-hydroxyethyl)-1H- imidazol-1-yl)but-3-yn-1-ol |
| 84 | 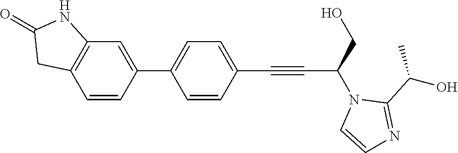 [SMILES: C[C@H](O)c1nccn1[C@@H](C#Cc1ccc(-c2ccc3c(c2)NC(=O)C3)cc1)CO] | 6-(4-((S)-4-hydroxy-3-(2-((S)-1- hydroxyethyl)-1H-imidazol-1-yl) but-1-yn-1-yl)phenyl)indolin-2-one |
| 85 | 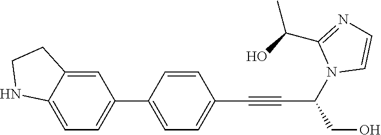 [SMILES: C[C@H](O)c1nccn1[C@@H](C#Cc1ccc(-c2ccc3c(c2)CCN3)cc1)CO] | (S)-2-(2-((S)-1-hydroxyethyl)-1H- imidazol-1-yl)-4-(4-(indolin-5- yl)phenyl)but-3-yn-1-ol |

In some embodiments, the compound is a compound of Formula (XII):

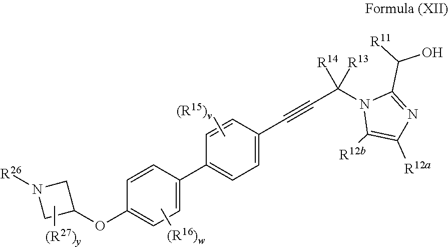

or a pharmaceutically acceptable salt, solvate, or stereoisomer thereof, wherein:
   R¹¹ is C₁-C₄ alkyl;
   R^{12a} and R^{12b} are each independently hydrogen, halogen, or C₁-C₄ alkyl;
   R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂;
   R¹⁴ is hydrogen or C₁-C₄ alkyl;
   each R¹⁵ and R¹⁶ is independently halogen, or C₁-C₄ alkyl;
   R²⁶ is C₁-C₆alkyl, C₁-C₆ fluoroalkyl, 4- to 6-membered heterocycloalkyl, heteroaryl, —C(═O)—C₁-C₆ alkyl, —C(═O)—N(R²⁸)₂, —CH₂—C(═O)—N(R²⁸)₂, or —S(O)₂—C₁-C₆ alkyl, wherein C₁-C₆ fluoroalkyl, C₁-C₆ alkyl, 4- to 6-membered heterocycloalkyl, and heteroaryl is unsubstituted or substituted with 1, 2, or 3 groups independently selected from halogen, C₁-C₆ alkyl, —OH, —NH₂, and —CN;
   each R²⁷ is independently hydrogen or C₁-C₆ alkyl;
   each R²⁸ is independently hydrogen or C₁-C₆ alkyl;
   or two R²⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo;
   y is 0, 1, or 2;
   v is 0, 1, or 2; and
   w is 0, 1, or 2.

In some embodiments of a compound of Formula (XII), R¹¹ is unsubstituted C₁-C₄ alkyl. In some embodiments, R¹¹ is C₁-C₂ alkyl. In some embodiments, R¹¹ is —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃. In some embodiments, R¹¹ is —CH₃ or —CH₂CH₃. In some embodiments, R¹¹ is —CH₃.

In some embodiments of a compound of Formula (XII), R^{12a} and R^{12b} are each independently R^{12a} and R^{12b} are each independently hydrogen, halogen, or unsubstituted C₁-C₄ alkyl. In some embodiments, R^{12a} and R^{12b} are each independently hydrogen, —F, —Cl, —Br, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments of a compound of Formula (XII), R^{12a} and R^{12b} are each independently hydrogen, —F, —Cl, —CH₃, —CH₂CH₃, or —CH(CH₃)₂.

In some embodiments of a compound of Formula (XII), R^{12a} is hydrogen. In some embodiments, R^{12b} is hydrogen. In some embodiments, R^{12a} and R^{12b} are each hydrogen.

In some embodiments of a compound of Formula (XII), R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂. In some embodiments, R¹³ is hydrogen. In some embodiments, R¹³ is —(C₁-C₄ alkylene)-OH. In some embodiments, R¹³ is —(C₁-C₂ alkylene)-OH.

In some embodiments of a compound of Formula (XII), R¹³ is —CH₂OH, —CH₂CH₂OH, —CH₂CH₂CH₂OH, —CH₂CH₂CH₂CH₂OH, —CH(CH₃)OH, —CH₂CH(CH₃)OH, —CH(CH₃)CH₂OH, —CH₂CH(CH₂CH₃)OH, or —CH(CH₂CH₃)CH₂OH. In some embodiments, R¹³ is —CH₂OH, —CH₂CH₂OH, or —CH(CH₃)OH. In some embodiments, R¹³ is —CH₂OH or —CH₂CH₂OH. In some embodiments, R¹³ is —CH₂OH.

In some embodiments of a compound of Formula (XII), R¹⁴ is hydrogen or unsubstituted C₁-C₄ alkyl. In some embodiments, R¹⁴ is hydrogen or C₁-C₂ alkyl. In some embodiments, R¹⁴ is hydrogen, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃. In some embodiments, R¹⁴ is hydrogen, —CH₃ or —CH₂CH₃. In some embodiments, R¹⁴ is hydrogen or —CH₃. In some embodiments, R¹⁴ is hydrogen. In some embodiments, R¹⁴ is —CH₃.

In some embodiments of a compound of Formula (XII), w is 0 and v is 0. In some embodiments, w is 0. In some embodiments, v is 0.

In some embodiments of a compound of Formula (XII), y is 0. In some embodiments, y is 1. In some embodiments, y is 1 and R²⁸ is C₁-C₃ alkyl. In some embodiments, y is 1 and R²⁸ is CH₃.

In some embodiments of a compound of Formula (XII), R²⁶ is C₁-C₆alkyl, C₁-C₆ fluoroalkyl, oxetanyl, imidazolyl, —C(═O)—CH₃, —S(O)₂—CH₃, —C(═O)—C₁-C₆ alkyl, —C(═O)—N(R²⁸)₂, —CH₂—C(═O)—NH(CH₃), wherein C₁-C₆ fluoroalkyl, C₁-C₆ alkyl, oxetanyl, and imidazolyl is unsubstituted or substituted with 1, 2, or 3 groups independently selected from halogen, —CH₃, —OH, —NH₂, and —CN; each R²⁸ is independently hydrogen or —CH₃; or two R²⁸ attached to the same nitrogen are taken together to form morpholinyl.

In some embodiments of a compound of Formula (XII), R²⁶ is

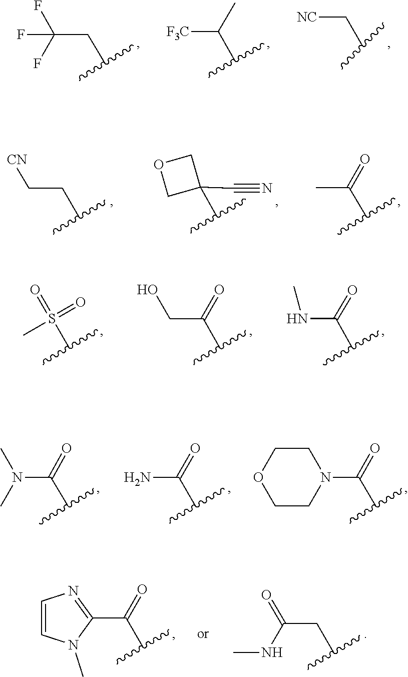

In some embodiments of a compound of Formula (XII), R²⁶ is

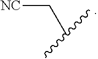 [SMILES: *CC#N]

In some embodiments, the compound is a compound of Table 4, or a pharmaceutically acceptable salt, solvate, or stereoisomer thereof. In some embodiments, the compound is a diastereomer of a compound of Table 4, or a pharmaceutically acceptable salt, solvate, or stereoisomer thereof.

TABLE 24

| Cmpd No | Structure | IUPAC Name |
|---|---|---|
| 86 | 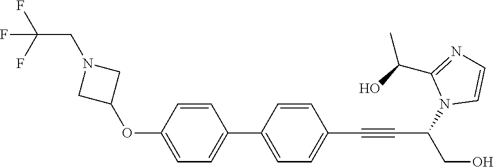 [SMILES: C[C@H](O)c1nccn1[C@@H](C#Cc1ccc(-c2ccc(OC3CN(CC(F)(F)F)C3)cc2)cc1)CO] | (S)-2-(2-((S)-1- hydroxyethyl)-1H-imidazol-1-yl)-4- (4′-((1-(2,2,2- trifluoroethyl)azetidin-3- yl)oxy)-[1,1′-biphenyl]-4- yl)but-3-yn-1-ol |
| 87 | 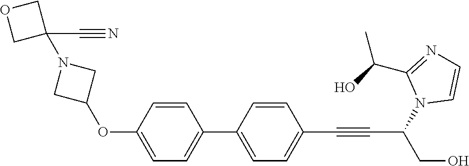 [SMILES: C[C@H](O)c1nccn1[C@@H](C#Cc1ccc(-c2ccc(OC3CN(C4(C#N)COC4)C3)cc2)cc1)CO] | 3-(3-((4′-((S)-4-hydroxy-3- (2-((S)-1-hydroxyethyl)-1H- imidazol-1-yl)but-1-yn-1- yl)-[1,1′-biphenyl]-4- yl)oxy)azetidin-1- yl)oxetane-3-carbonitrile |
| 88 | 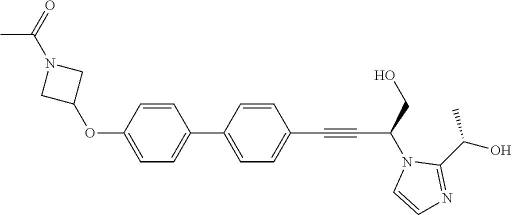 [SMILES: CC(=O)N1CC(Oc2ccc(-c3ccc(C#C[C@@H](CO)n4ccnc4[C@H](C)O)cc3)cc2)C1] | 1-(3-((4′-((S)-4-hydroxy-3- (2-((S)-1-hydroxyethyl)-1H- imidazol-1-yl)but-1-yn-1- yl)-[1,1′-biphenyl]-4- yl)oxy)azetidin-1-yl)ethan- 1-one |
| 89 | 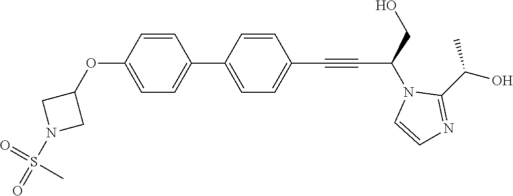 [SMILES: C[C@H](O)c1nccn1[C@@H](C#Cc1ccc(-c2ccc(OC3CN(S(C)(=O)=O)C3)cc2)cc1)CO] | (S)-2-(2-((S)-1- hydroxyethyl)-1H-imidazol- 1-yl)-4-(4′-((1- (methylsulfonyl)azetidin-3- yl)oxy)-[1,1′-biphenyl]-4- yl)but-3-yn-1-ol |
| 90 | 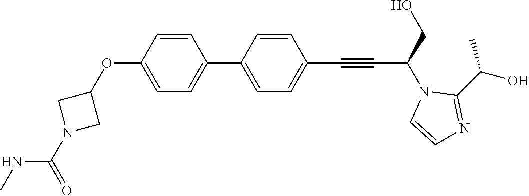 [SMILES: CNC(=O)N1CC(Oc2ccc(-c3ccc(C#C[C@@H](CO)n4ccnc4[C@H](C)O)cc3)cc2)C1] | 3-((4′-((S)-4-hydroxy-3-(2- ((S)-1-hydroxyethyl)-1H- imidazol-1-yl)but-1-yn-1- yl)-[1,1′-biphenyl]-4- yl)oxy)-N-methylazetidine- 1-carboxamide |
| 91 | 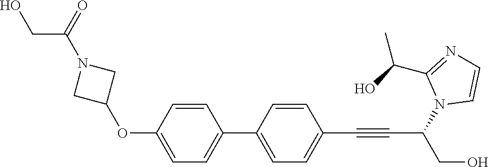 [SMILES: C[C@H](O)c1nccn1[C@@H](C#Cc1ccc(-c2ccc(OC3CN(C(=O)CO)C3)cc2)cc1)CO] | 2-hydroxy-1-(3-((4′-((S)-4- hydroxy-3-(2-((S)-1- hydroxyethyl)-1H-imidazol- 1-yl)but-1-yn-1-yl)-[1,1′- biphenyl]-4-yl)oxy)azetidin- 1-yl)ethan-1-one |
| 92 | 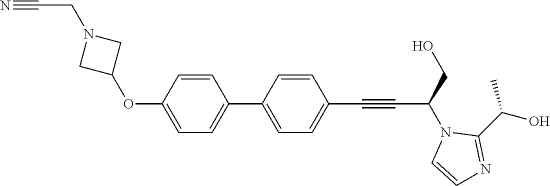 [SMILES: C[C@H](O)c1nccn1[C@@H](C#Cc1ccc(-c2ccc(OC3CN(CC#N)C3)cc2)cc1)CO] | 2-(3-((4′-((S)-4-hydroxy-3- (2-((S)-1-hydroxyethyl)-1H- imidazol-1-yl)but-1-yn-1- yl)-[1,1′-biphenyl]-4- yl)oxy)azetidin-1- yl)acetonitrile |
| 93 | 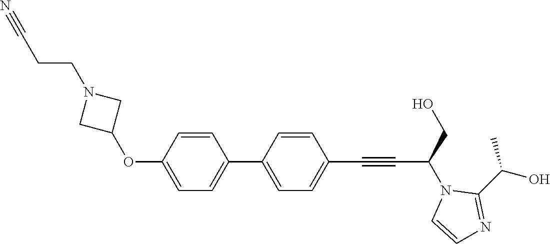 [SMILES: C[C@H](O)c1nccn1[C@@H](C#Cc1ccc(-c2ccc(OC3CN(CCC#N)C3)cc2)cc1)CO] | 3-(3-((4′-((S)-4-hydroxy-3- (2-((S)-1-hydroxyethyl)-1H- imidazol-1-yl)but-1-yn-1- yl)-[1,1′-biphenyl]-4- yl)oxy)azetidin-1- yl)propanenitrile |
| 94 | 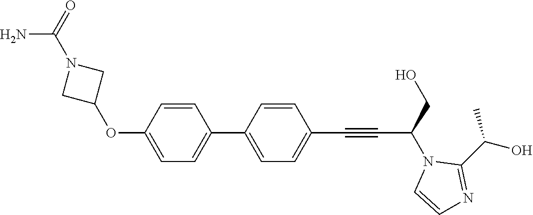 [SMILES: C[C@H](O)c1nccn1[C@@H](C#Cc1ccc(-c2ccc(OC3CN(C(N)=O)C3)cc2)cc1)CO] | 3-((4′-((S)-4-hydroxy-3-(2- ((S)-1-hydroxyethyl)-1H- imidazol-1-yl)but-1-yn-1- yl)-[1,1′-biphenyl]-4- yl)oxy)azetidine-1- carboxamide |
| 95 | 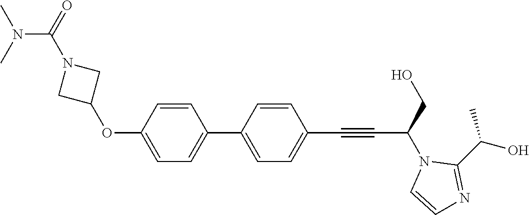 [SMILES: C[C@H](O)c1nccn1[C@@H](C#Cc1ccc(-c2ccc(OC3CN(C(=O)N(C)C)C3)cc2)cc1)CO] | 3-((4′-((S)-4-hydroxy-3-(2- ((S)-1-hydroxyethyl)-1H- imidazol-1-yl)but-1-yn-1- yl)-[1,1′-biphenyl]-4- yl)oxy)-N,N- dimethylazetidine-1- carboxamide |
| 96 | 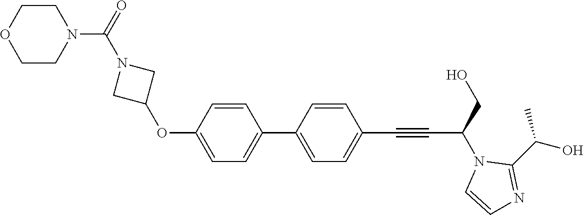 [SMILES: C[C@H](O)c1nccn1[C@@H](C#Cc1ccc(-c2ccc(OC3CN(C(=O)N4CCOCC4)C3)cc2)cc1)CO] | (3-((4′-((S)-4-hydroxy-3-(2- ((S)-1-hydroxyethyl)-1H- imidazol-1-yl)but-1-yn-1- yl)-[1,1′-biphenyl]-4- yl)oxy)azetidin-1- yl)(morpholino)methanone |
| 97 | 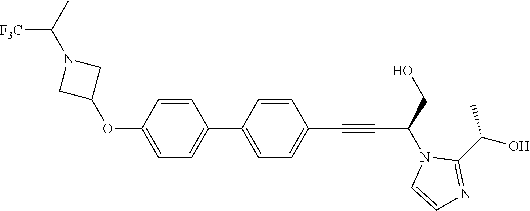 [SMILES: CC(N1CC(Oc2ccc(-c3ccc(C#C[C@@H](CO)n4ccnc4[C@H](C)O)cc3)cc2)C1)C(F)(F)F] | (2S)-2-(2-((S)-1- hydroxyethyl)-1H-imidazol- 1-yl)-4-(4′-((1-(1,1,1- trifluoropropan-2- yl)azetidin-3-yl)oxy)-[1,1′- biphenyl]-4-yl)but-3-yn-1- ol |
| 98 | 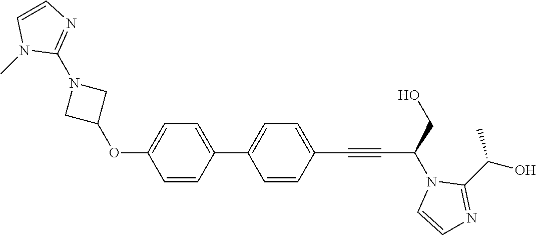 [SMILES: C[C@H](O)c1nccn1[C@@H](C#Cc1ccc(-c2ccc(OC3CN(c4nccn4C)C3)cc2)cc1)CO] | (S)-2-(2-((S)-1- hydroxyethyl)-1H-imidazol- 1-yl)-4-(4′-((1-(1-methyl- 1H-imidazol-2-yl)azetidin- 3-yl)oxy)-[1,1′-biphenyl]-4- yl)but-3-yn-1-ol |
| 99 | 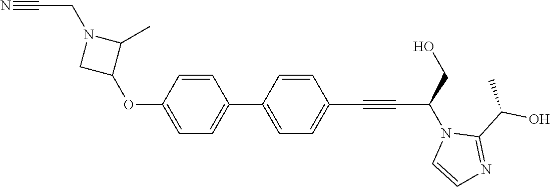 [SMILES: CC1C(Oc2ccc(-c3ccc(C#C[C@@H](CO)n4ccnc4[C@H](C)O)cc3)cc2)CN1CC#N] | 2-(3-((4′-(S)-4-hydroxy-3- (2-((S)-1-hydroxyethyl)-1H- imidazol-1-yl)but-1-yn-1- yl)-[1,1′-biphenyl]-4- yl)oxy)-2-methylazetidin-1- yl)acetonitrile |
| 100 | 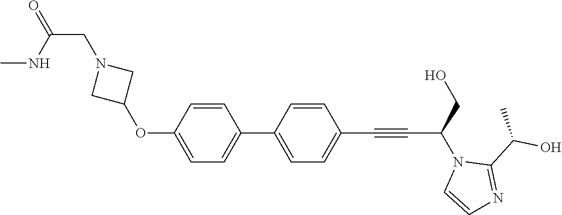 [SMILES: CNC(=O)CN1CC(Oc2ccc(-c3ccc(C#C[C@@H](CO)n4ccnc4[C@H](C)O)cc3)cc2)C1] | 2-(3-((4′-((S)-4-hydroxy-3- (2-((S)-1-hydroxyethyl)-1H- imidazol-1-yl)but-1-yn-1- yl)-[1,1′-biphenyl]-4- yl)oxy)azetidin-1-yl)-N- methylacetamide |

In some embodiments, the compound is a compound of Table 5, or a pharmaceutically acceptable salt, solvate, or stereoisomer thereof. In some embodiments, the compound is a diastereomer of a compound of Table 5, or a pharmaceutically acceptable salt, solvate, or stereoisomer thereof.

TABLE 5

| Cmpd | | |
|---|---|---|
| No | Structure | IUPAC Name |
| 101 | 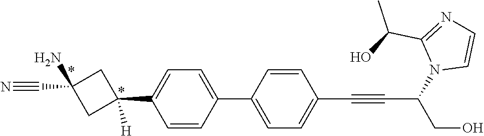 [SMILES: C[C@H](O)c1nccn1[C@@H](C#Cc1ccc(-c2ccc([C@H]3C[C@](N)(C#N)C3)cc2)cc1)CO] | (rac-cis)-1-amino-3-(4′-((S)-4- hydroxy-3-(2-((S)-1- hydroxyethyl)-1H-imidazol-1- yl)but-1-yn-1-yl)-[1,1′-biphenyl]- 4-yl)cyclobutane-1-carbonitrile |
| | *racemic-cis | |
| 102 | 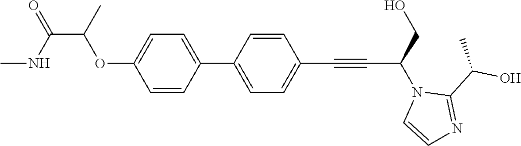 [SMILES: CNC(=O)C(C)Oc1ccc(-c2ccc(C#C[C@@H](CO)n3ccnc3[C@H](C)O)cc2)cc1] | 2-((4′-((S)-4-hydroxy-3-(2-((S)-1- hydroxyethyl)-1H-imidazol-1- yl)but-1-yn-1-yl)-[1,1′-biphenyl]- 4-yl)oxy)-N-methylpropanamide |
| 103 | 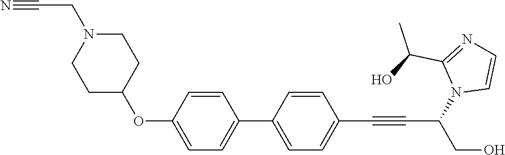 [SMILES: C[C@H](O)c1nccn1[C@@H](C#Cc1ccc(-c2ccc(OC3CCN(CC#N)CC3)cc2)cc1)CO] | 2-(4-((4′-((S)-4-hydroxy-3-(2-((S)- 1-hydroxyethyl)-1H-imidazol-1- yl)but-1-yn-1-yl)-[1,1′-biphenyl]- 4- yl)oxy)piperidin-1-yl)acetonitrile |
| 104 | 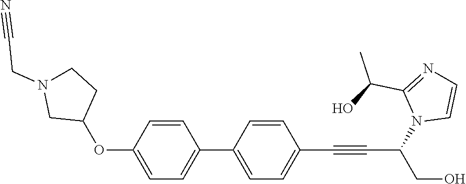 [SMILES: C[C@H](O)c1nccn1[C@@H](C#Cc1ccc(-c2ccc(OC3CCN(CC#N)C3)cc2)cc1)CO] | 2-(3-((4′-((S)-4-hydroxy-3-(2-((S)- 1-hydroxyethyl)-1H-imidazol-1- yl)but-1-yn-1-yl)-[1,1′-biphenyl]- 4-yl)oxy) pyrrolidin-1-yl)acetonitrile |
| 105 | 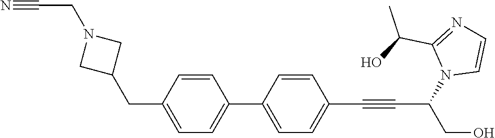 [SMILES: C[C@H](O)c1nccn1[C@@H](C#Cc1ccc(-c2ccc(CC3CN(CC#N)C3)cc2)cc1)CO] | 2-(3-((4′-((S)-4-hydroxy-3-(2-((S)- 1-hydroxyethyl)-1H-imidazol-1- yl)but-1-yn-1-yl)-[1,1′-biphenyl]- 4-yl)methyl) azetidin-1-yl)acetonitrile |
| 106 | 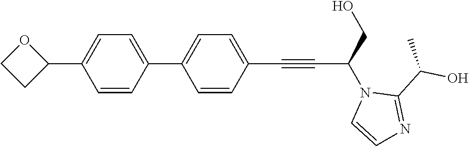 [SMILES: C[C@H](O)c1nccn1[C@@H](C#Cc1ccc(-c2ccc(C3CCO3)cc2)cc1)CO] | (2S)-2-(2-((S)-1-hydroxyethyl)- 1H-imidazol-1-yl)-4-(4′-(oxetan-2- yl)-[1,1′-biphenyl]-4-yl)but-3-yn- 1-ol |
| 107 | 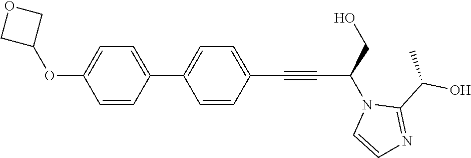 [SMILES: C[C@H](O)c1nccn1[C@@H](C#Cc1ccc(-c2ccc(OC3COC3)cc2)cc1)CO] | (S)-2-(2-((S)-1-hydroxyethyl)-1H- imidazol-1-yl)-4-(4′-(oxetan-3- yloxy)-[1,1′-biphenyl]-4-yl)but-3- yn-1-ol |
| 108 | 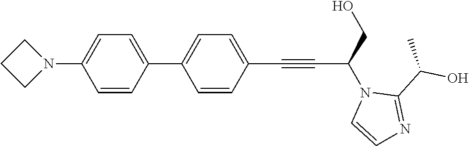 [SMILES: C[C@H](O)c1nccn1[C@@H](C#Cc1ccc(-c2ccc(N3CCC3)cc2)cc1)CO] | (S)-4-(4′-(azetidin-1-yl)-[1,1′- biphenyl]-4-yl)-2-(2-((S)-1- hydroxyethyl)-1H-imidazol-1- yl)but-3-yn-1-ol |
| 109 | 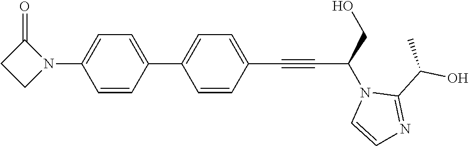 [SMILES: C[C@H](O)c1nccn1[C@@H](C#Cc1ccc(-c2ccc(N3CCC3=O)cc2)cc1)CO] | 1-(4′-((S)-4-hydroxy-3-(2-((S)-1- hydroxyethyl)-1H-imidazol-1- yl)but-1-yn-1-yl)-[1,1′-biphenyl]- 4-yl)azetidin-2-one |
| 110 | 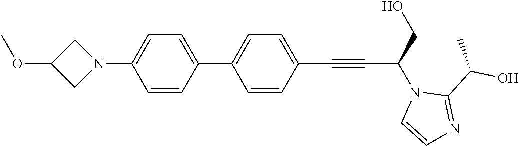 [SMILES: COC1CN(c2ccc(-c3ccc(C#C[C@@H](CO)n4ccnc4[C@H](C)O)cc3)cc2)C1] | (S)-2-(2-((S)-1-hydroxyethyl)-1H- imidazol-1-yl)-4-(4′-(3- methoxyazetidin-1-yl)-[1,1′- biphenyl]-4-yl)but-3-yn-1-ol |
| 111 | 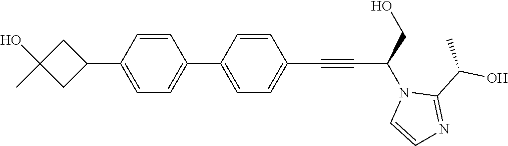 [SMILES: C[C@H](O)c1nccn1[C@@H](C#Cc1ccc(-c2ccc(C3CC(C)(O)C3)cc2)cc1)CO] | 3-(4′-((S)-4-hydroxy-3-(2-((S)-1- hydroxyethyl)-1H-imidazol-1- yl)but-1-yn-1-yl)-[1,1′-biphenyl]- 4-yl)-1-methylcyclobutan-1-ol |
| 112 | 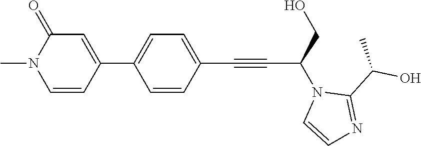 [SMILES: C[C@H](O)c1nccn1[C@@H](C#Cc1ccc(-c2ccn(C)c(=O)c2)cc1)CO] | 4-(4-((S)-4-hydroxy-3-(2-((S)-1- hydroxyethyl)-1H-imidazol-1- yl)but-1-yn-1-yl)phenyl)-1- methylpyridin-2(1H)-one |
| 113** | 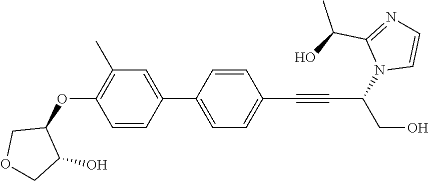 [SMILES: Cc1cc(-c2ccc(C#C[C@@H](CO)n3ccnc3[C@H](C)O)cc2)ccc1O[C@@H]1COC[C@H]1O] | (3R,4R)-4-((4′-((S)-4-hydroxy-3- (2-((S)-1-hydroxyethyl)-1H- imidazol-1-yl)but-1-yn-1-yl)-3- methyl-[1,1′-biphenyl]-4- yl)oxy)tetrahydrofuran-3-ol |
| 114** | 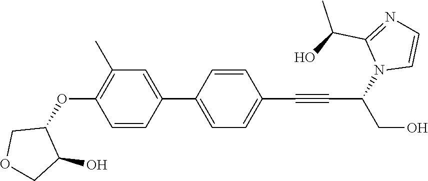 [SMILES: Cc1cc(-c2ccc(C#C[C@@H](CO)n3ccnc3[C@H](C)O)cc2)ccc1O[C@H]1COC[C@@H]1O] | (3S,4S)-4-((4′-((S)-4-hydroxy-3- (2-((S)-1-hydroxyethyl)- 1H-imidazol-1-yl)but- 1-yn-1-yl)-3-methyl-[1,1′- biphenyl]-4- yl)oxy)tetrahydrofuran-3-ol |
| 115** | 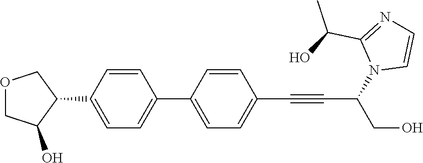 [SMILES: C[C@H](O)c1nccn1[C@@H](C#Cc1ccc(-c2ccc([C@H]3COC[C@@H]3O)cc2)cc1)CO] | (3R,4S)-4-(4′-((S)-4-hydroxy-3- (2-((S)-1-hydroxyethyl)- 1H-imidazol- 1-yl)but-1-yn-1-yl)-[1,1′- biphenyl]-4-yl) tetrahydrofuran-3-ol |
| 116** | 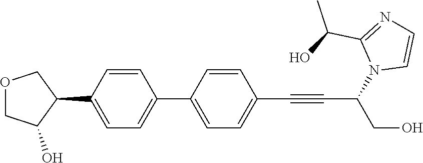 [SMILES: C[C@H](O)c1nccn1[C@@H](C#Cc1ccc(-c2ccc([C@@H]3COC[C@H]3O)cc2)cc1)CO] | (3S,4R)-4-(4′-((S)-4-hydroxy-3- (2-((S)-1-hydroxyethyl)- 1H-imidazol-1-yl)but-1-yn- 1-yl)-[1,1′-biphenyl]- 4-yl)tetrahydrofuran-3-ol |
| 117 | 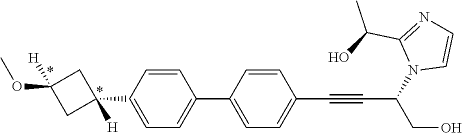 [SMILES: CO[C@H]1C[C@H](c2ccc(-c3ccc(C#C[C@@H](CO)n4ccnc4[C@H](C)O)cc3)cc2)C1] | (S)-2-(2-((S)-1-hydroxyethyl)-1H- imidazol-1-yl)-4-(4′-((rac-trans)- 3- methoxycyclobutyl)-[1,1′- biphenyl]-4-yl)but-3-yn-1-ol |
| | *racemic-trans | |
| 118 | 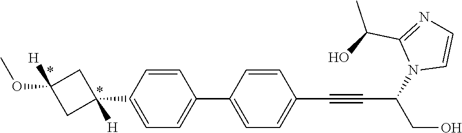 [SMILES: CO[C@H]1C[C@@H](c2ccc(-c3ccc(C#C[C@@H](CO)n4ccnc4[C@H](C)O)cc3)cc2)C1] | (S)-2-(2-((S)-1-hydroxyethyl)-1H- imidazol-1-yl)-4-(4′-((rac-cis)-3- methoxycyclobutyl)-[1,1′- biphenyl]-4-yl)but-3-yn-1-ol |
| | *racemic-cis | |
| 119 | 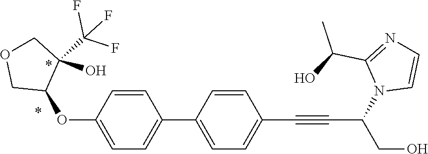 [SMILES: C[C@H](O)c1nccn1[C@@H](C#Cc1ccc(-c2ccc(O[C@H]3COC[C@]3(O)C(F)(F)F)cc2)cc1)CO] | (rac-cis)-4-((4′-((S)-4-hydroxy-3- (2-((S)-1-hydroxyethyl)-1H- imidazol-1-yl)but-1-yn-1-yl)- [1,1′-biphenyl]-4-yl)oxy)-3- (trifluoromethyl)tetrahydrofuran- 3-ol |
| | *racemic-cis | |
| 120 | 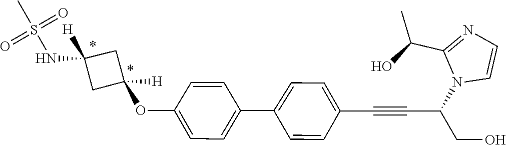 [SMILES: C[C@H](O)c1nccn1[C@@H](C#Cc1ccc(-c2ccc(O[C@H]3C[C@H](NS(C)(=O)=O)C3)cc2)cc1)CO] | N-((rac-trans)-3-((4′-((S)-4- hydroxy-3-(2-((S)-1- hydroxyethyl)-1H-imidazol-1- yl)but-1-yn-1-yl)-[1,1′-biphenyl]- 4-yl)oxy)cyclobutyl)methane- sulfonamide |
| | *racemic-trans | |
| 121 | 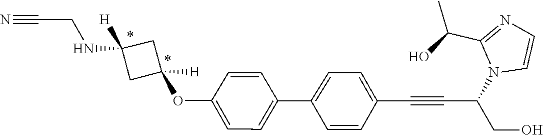 [SMILES: C[C@H](O)c1nccn1[C@@H](C#Cc1ccc(-c2ccc(O[C@H]3C[C@H](NCC#N)C3)cc2)cc1)CO] | 2-(((rac-trans)-3-((4′-((S)-4- hydroxy-3-(2-((S)-1- hydroxyethyl)-1H-imidazol-1- yl)but-1-yn-1-yl)-[1,1′-biphenyl]- 4-yl)oxy)cyclobutyl)amino) acetonitrile |
| | *racemic-trans | |
| 122 | 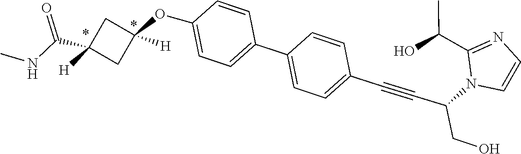 [SMILES: CNC(=O)[C@H]1C[C@H](Oc2ccc(-c3ccc(C#C[C@@H](CO)n4ccnc4[C@H](C)O)cc3)cc2)C1] | (rac-trans)-3-((4′-((S)-4-hydroxy- 3-(2-((S)-1-hydroxyethyl)-1H- imidazol-1-yl)but-1-yn-1-yl)- [1,1′-biphenyl]-4-yl)oxy)-N- methylcyclobutane-1- carboxamide |
| | *racemic-trans | |
| 123 | 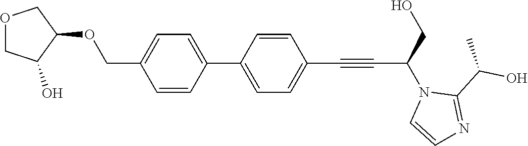 [SMILES: C[C@H](O)c1nccn1[C@@H](C#Cc1ccc(-c2ccc(CO[C@@H]3COC[C@H]3O)cc2)cc1)CO] | (3R,4R)-4-((4′-((S)-4-hydroxy-3- (2-((S)-1-hydroxyethyl)-1H- imidazol-1-yl)but-1-yn-1-yl)-[1,1′- biphenyl]-4- yl)methoxy)tetrahydrofuran-3-ol |
| 124 | 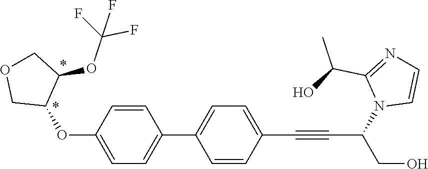 [SMILES: C[C@H](O)c1nccn1[C@@H](C#Cc1ccc(-c2ccc(O[C@@H]3COC[C@H]3OC(F)(F)F)cc2)cc1)CO] | (S)-2-(2-((S)-1-hydroxyethyl)-1H- imidazol-1-yl)-4-(4′-(((rac-trans)- 4-(trifluoromethoxy)- tetrahydrofuran-3-yl)oxy)-[1,1′- biphenyl]-4-yl)but-3-yn-1-ol |
| | *racemic-trans | |
| 125** | 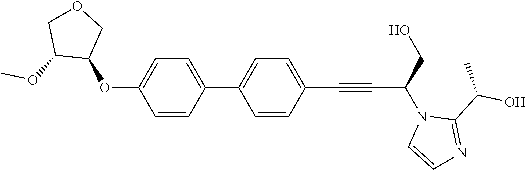 [SMILES: CO[C@@H]1COC[C@H]1Oc1ccc(-c2ccc(C#C[C@@H](CO)n3ccnc3[C@H](C)O)cc2)cc1] | (S)-2-(2-((S)-1-hydroxyethyl)-1H- imidazol-1-yl)-4-(4′-(((3R,4R)-4- methoxytetrahydrofuran-3-yl)oxy)- [1,1′-biphenyl]-4-yl)but-3-yn-1-ol |
| 126** | 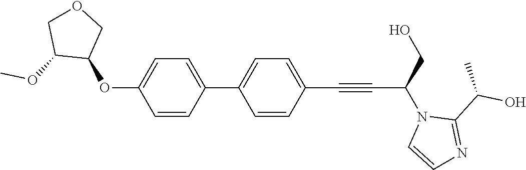 [SMILES: CO[C@@H]1COC[C@H]1Oc1ccc(-c2ccc(C#C[C@@H](CO)n3ccnc3[C@H](C)O)cc2)cc1] | (S)-2-(2-((S)-1-hydroxyethyl)-1H- imidazol-1-yl)-4-(4′-(((3R,4R)-4- methoxytetrahydrofuran- 3-yl)oxy)-[1,1′-biphenyl]- 4-yl)but-3-yn-1-ol |
| 127 | 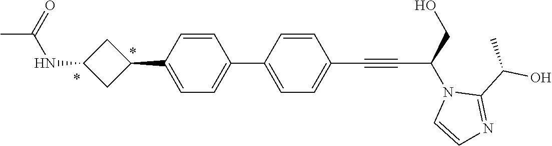 [SMILES: CC(=O)N[C@H]1C[C@H](c2ccc(-c3ccc(C#C[C@@H](CO)n4ccnc4[C@H](C)O)cc3)cc2)C1] | N-((rac-trans)-3-(4′-((S)-4- hydroxy-3-(2-((S)-1- hydroxyethyl)-1H-imidazol-1- yl)but-1-yn-1-yl)-[1,1′-biphenyl]- 4-yl)cyclobutyl)acetamide |
| | *racemic-trans | |
| 128 | 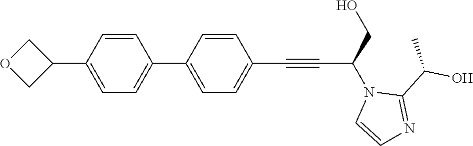 [SMILES: C[C@H](O)c1nccn1[C@@H](C#Cc1ccc(-c2ccc(C3COC3)cc2)cc1)CO] | (S)-2-(2-((S)-1-hydroxyethyl)- 1H-imidazol-1-yl)- 4-(4′-(oxetan-3-yl)-[1,1′- biphenyl]-4-yl)but-3-yn-1-ol |
| 129 | 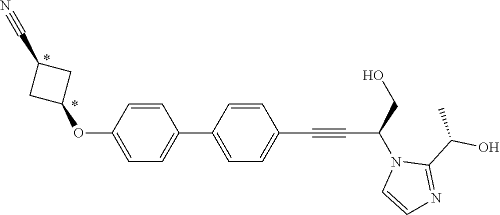 [SMILES: C[C@H](O)c1nccn1[C@@H](C#Cc1ccc(-c2ccc(O[C@H]3C[C@@H](C#N)C3)cc2)cc1)CO] | (rac-cis)-3-((4′-((S)-4-hydroxy-3- (2-((S)-1-hydroxyethyl)-1H- imidazol-1-yl)but-1-yn-1-yl)- [1,1′-biphenyl]-4-yl) oxy)cyclobutane-1- carbonitrile |
| | *racemic-cis | |
| 130 | 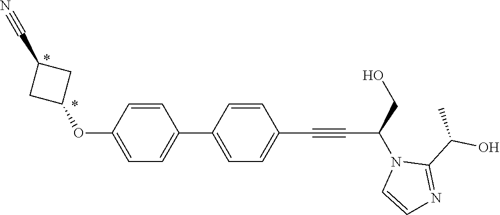 [SMILES: C[C@H](O)c1nccn1[C@@H](C#Cc1ccc(-c2ccc(O[C@H]3C[C@H](C#N)C3)cc2)cc1)CO] | (rac-trans)-3-((4′-((S)-4-hydroxy- 3-(2-((S)-1-hydroxyethyl)-1H- imidazol-1-yl)but-1-yn-1-yl)- [1,1′-biphenyl]-4-yl)oxy) cyclobutane-1-carbonitrile |
| | *racemic-trans | |
| 131 | 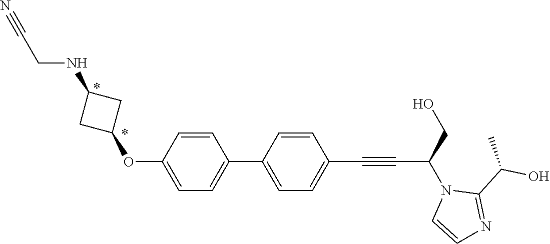 [SMILES: C[C@H](O)c1nccn1[C@@H](C#Cc1ccc(-c2ccc(O[C@H]3C[C@@H](NCC#N)C3)cc2)cc1)CO] | 2-((rac-cis)-3-((4′-((S)-4-hydroxy- 3-(2-((S)-1-hydroxyethyl)-1H- imidazol-1-yl)but-1-yn-1-yl)- [1,1′-biphenyl]-4- yl)oxy)cyclobutyl)amino) acetonitrile |
| | *racemic-cis | |
| 132 | 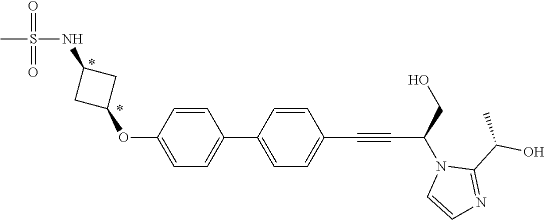 [SMILES: C[C@H](O)c1nccn1[C@@H](C#Cc1ccc(-c2ccc(O[C@H]3C[C@@H](NS(C)(=O)=O)C3)cc2)cc1)CO] | N-((rac-cis)-3-((4′-((S)-4- hydroxy-3-(2-((S)-1- hydroxyethyl)-1H- imidazol-1-yl)but-1-yn-1-yl)- [1,1′-biphenyl]-4- yl)oxy)cyclobutyl)methane- sulfonamide |
| | *racemic-cis | |
| 133 | 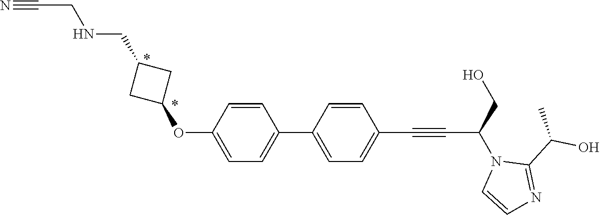 [SMILES: C[C@H](O)c1nccn1[C@@H](C#Cc1ccc(-c2ccc(O[C@H]3C[C@H](CNCC#N)C3)cc2)cc1)CO] | 2-((((rac-trans)-3-((4′-((S)-4- hydroxy-3-(2-((S)-1- hydroxyethyl)-1H-imidazol-1- yl)but-1-yn-1-yl)-[1,1′-biphenyl]- 4-yl)oxy)cyclobutyl) methyl)amino) acetonitrile |
| | *racemic-trans | |
| 134 | 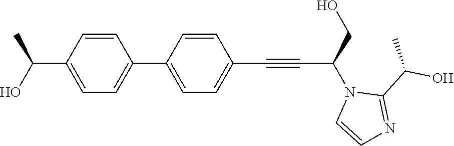 [SMILES: C[C@H](O)c1ccc(-c2ccc(C#C[C@@H](CO)n3ccnc3[C@H](C)O)cc2)cc1] | (S)-2-(2-((S)-1-hydoxyethyl)-1H- imidazol-1-yl)-4-(4′-((S)-1- hydroxyethyl)-[1,1′-biphenyl]-4- yl)but-3-yn-1-ol |
| 135 | 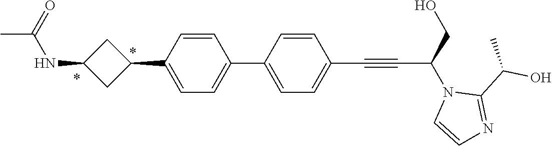 [SMILES: CC(=O)N[C@H]1C[C@@H](c2ccc(-c3ccc(C#C[C@@H](CO)n4ccnc4[C@H](C)O)cc3)cc2)C1] | N-((rac-cis)-3-(4′-((S)-4-hydroxy- 3-(2-((S)-1-hydroxyethyl)-1H- imidazol-1-yl)but-1-yn-1-yl)- [1,1′-biphenyl]-4- yl)cyclobutyl)acetamide |
| | *racemic-cis | |
| 136 | 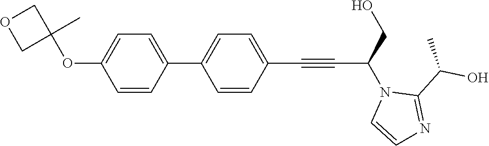 [SMILES: C[C@H](O)c1nccn1[C@@H](C#Cc1ccc(-c2ccc(OC3(C)COC3)cc2)cc1)CO] | (S)-2-(2-((S)-1-hydroxyethyl)-1H- imidazol-1-yl)-4-(4′-((3- methyloxetan-3-yl)oxy)-[1,1′- biphenyl]-4-yl)but-3-yn-1-ol |
| 137 | 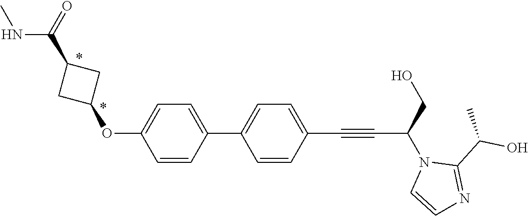 [SMILES: CNC(=O)[C@H]1C[C@@H](Oc2ccc(-c3ccc(C#C[C@@H](CO)n4ccnc4[C@H](C)O)cc3)cc2)C1] | (rac-cis)-3-((4′-((S)-4-hydroxy-3- (2-((S)-1-hydroxyethyl)-1H- imidazol-1-yl)but-1-yn-1-yl)- [1,1′-biphenyl]-4-yl)oxy)-N- methylcyclobutane-1- carboxamide |
| | *racemic-cis | |
| 138 | 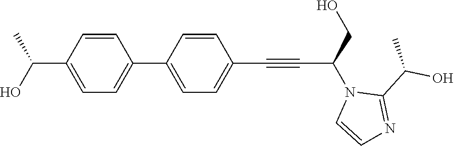 [SMILES: C[C@H](O)c1nccn1[C@@H](C#Cc1ccc(-c2ccc([C@@H](C)O)cc2)cc1)CO] | (S)-2-(2-((S)-1-hydroxyethyl)- 1H- imidazol-1-yl)-4-(4′-((R)-1- hydroxyethyl)-[1,1′-biphenyl]-4- yl)but-3-yn-1-ol |
| 139 | 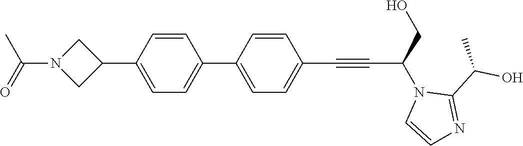 [SMILES: CC(=O)N1CC(c2ccc(-c3ccc(C#C[C@@H](CO)n4ccnc4[C@H](C)O)cc3)cc2)C1] | 1-(3-(4′-((S)-4-hydroxy-3-(2-((S)- 1-hydroxyethyl)-1H-imidazol-1- yl)but-1-yn-1-yl)-[1,1′-biphenyl]- 4-yl)azetidin-1- yl)ethan-1-one |
| 140 | 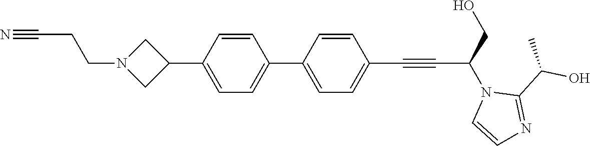 [SMILES: C[C@H](O)c1nccn1[C@@H](C#Cc1ccc(-c2ccc(C3CN(CCC#N)C3)cc2)cc1)CO] | 3-(3-(4′-((S)-4-hydroxy-3-(2-((S)- 1-hydroxyethyl)-1H-imidazol-1- yl)but-1-yn-1-yl)-[1,1′-biphenyl]- 4-yl)azetidin-1- yl)propanenitrile |
| 141 | 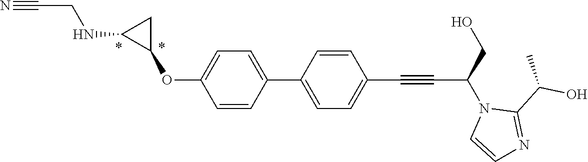 [SMILES: C[C@H](O)c1nccn1[C@@H](C#Cc1ccc(-c2ccc(O[C@@H]3C[C@H]3NCC#N)cc2)cc1)CO] | 2-(((rac-trans)-2-((4′-((S)-4- hydroxy-3-(2-((S)-1- hydroxyethyl)-1H-imidazol-1- yl)but-1-yn-1-yl)-[1,1′-biphenyl]- 4-yl)oxy)cyclopropyl)amino) acetonitrile |
| | *racemic-trans | |
| 142 | 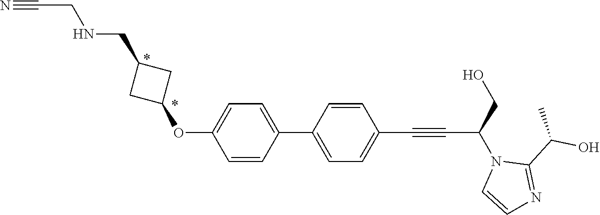 [SMILES: C[C@H](O)c1nccn1[C@@H](C#Cc1ccc(-c2ccc(O[C@H]3C[C@@H](CNCC#N)C3)cc2)cc1)CO] | 2-((((rac-cis)-3-((4′-((S)-4- hydroxy-3-(2-((S)-1- hydroxyethyl)-1H-imidazol-1- yl)but-1-yn-1-yl)-[1,1′-biphenyl]- 4-yl)oxy)cyclobutyl) methyl)amino)acetonitrile |
| | *racemic-cis | |
| 143 | 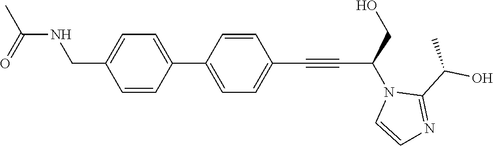 [SMILES: CC(=O)NCc1ccc(-c2ccc(C#C[C@@H](CO)n3ccnc3[C@H](C)O)cc2)cc1] | N-((4′-((S)-4-hydroxy-3-(2-((S)-1- hydroxyethyl)-1H-imidazol-1- yl)but-1-yn-1-yl)-[1,1′-biphenyl]- 4-yl)methyl)acetamide |

**stereochemistry arbitrarily assigned. After chiral separation, single stereoisomers are isolated but the absolute configuration of the stereochemical
center is unknown.

Any combination of the groups described above for the various variables is contemplated herein. Throughout the specification, groups and substituents thereof are chosen by one skilled in the field to provide stable moieties and compounds.

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic at the concentration or amount used, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound of Formulas (I), (II), (III), (IV), (V), (VI), (VII), (VIII), (IX), (X), (XI), or (XII) with an acid. In some embodiments, the compound of Formulas (I), (II), (III), (IV), (V), (VI), (VII), (VIII), (IX), (X), (XI), or (XII) (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxy ethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound of Formulas (I), (II), (III), (IV), (V), (VI), (VII), (VIII), (IX), (X), (XI), or (XII) is prepared as a chloride salt, sulfate salt, bromide salt, mesylate salt, maleate salt, citrate salt or phosphate salt.

In some embodiments, pharmaceutically acceptable salts are obtained by reacting a compound of Formulas (I), (II), (III), (IV), (V), (VI), (VII), (VIII), (IX), (X), (XI), or (XII) with a base. In some embodiments, the compound of Formulas (I), (II), (III), (IV), (V), (VI), (VII), (VIII), (IX), (X), (XI), or (XII) is acidic and is reacted with a base. In such situations, an acidic proton of the compound of Formulas (I), (II), (III), (IV), (V), (VI), (VII), (VIII), (IX), (X), (XI), or (XII) is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), or pharmaceutically acceptable salts of compounds having the structure of Formulas (I), (II), (III), (IV), (V), (VI), (VII), (VIII), (IX), (X), (XI), or (XII), as well as active metabolites of these compounds having the same type of activity.

In some embodiments, sites on the organic radicals (e.g. alkyl groups, aromatic rings) of compounds of Formulas (I), (II), (III), (IV), (V), (VI), (VII), (VIII), (IX), (X), (XI), or (XII) are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the compounds of Formulas (I), (II), (III), (IV), (V), (VI), (VII), (VIII), (IX), (X), (XI), or (XII) possess one or more stereocenters and each stereocenter exists independently in either the R or S configuration. In some embodiments, the compound of Formulas (I), (II), (III), (IV), (V), (VI), (VII), (VIII), (IX), (X), (XI), or (XII) exists in the R configuration. In some embodiments, the compound of Formulas (I), (II), (III), (IV), (V), (VI), (VII), (VIII), (IX), (X), (XI), or (XII) exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds of Formulas (I), (II), (III), (IV), (V), (VI), (VII), (VIII), (IX), (X), (XI), or (XII) are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, N-alkyloxyacyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a compound of Formulas (I), (II), (III), (IV), (V), (VI), (VII), (VIII), (IX), (X), (XI), or (XII) as set forth herein are included within the scope of the claims. In some cases, some of the herein-described compounds is a prodrug for another derivative or active compound.

In some embodiments, any one of the hydroxyl group(s), amino group(s) and/or carboxylic acid group(s) are functionalized in a suitable manner to provide a prodrug moiety. In some embodiments, the prodrug moiety is as described above.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulfhydryl groups. Metabolites of the compounds disclosed herein are optionally identified either by administration of compounds to a host and analysis of tissue samples from the host, or by incubation of compounds with hepatic cells in vitro and analysis of the resulting compounds.

In some instances, heterocyclic rings may exist in tautomeric forms. In such situations, it is understood that the structures of said compounds are illustrated or named in one tautomeric form but could be illustrated or named in the alternative tautomeric form. The alternative tautomeric forms are expressly included in this disclosure, such as, for example, the structures illustrated below. For example, benzimidazoles or imidazoles could exist in the following tautomeric forms:

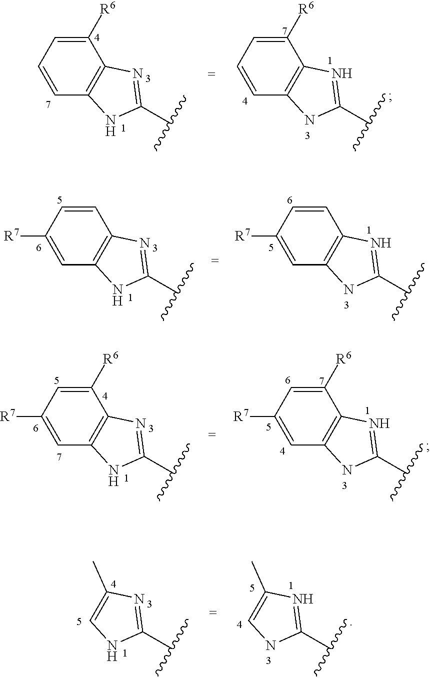

### Preparation of Compounds

Compounds of Formulas (I), (II), (III), (IV), (V), (VI), (VII), (VIII), (IX), (X), (XI), or (XII) described herein are synthesized using standard synthetic techniques or using methods known in the art in combination with methods described herein.

Unless otherwise indicated, conventional methods of mass spectroscopy, NMR, HPLC are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions.

In some embodiments, compounds described herein are prepared as described in Scheme A.

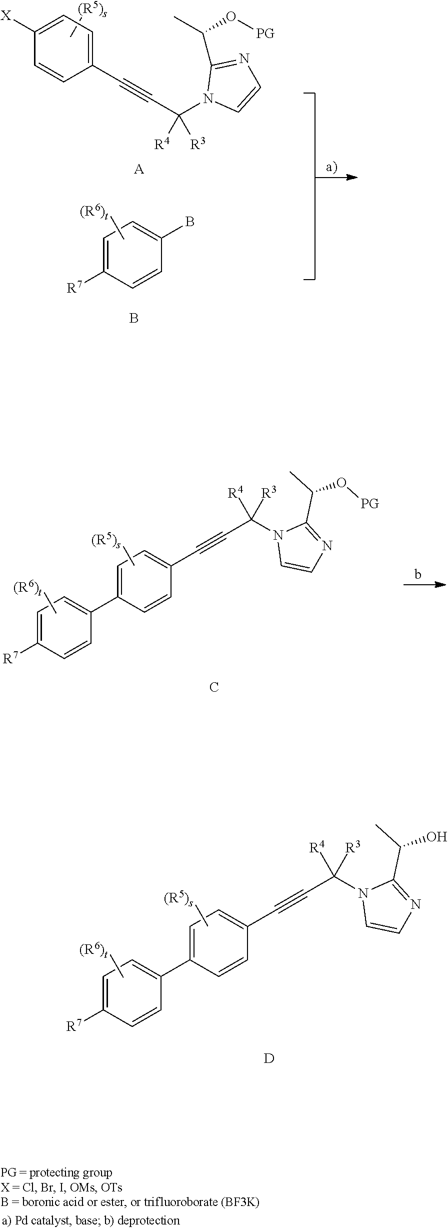

An organometallic coupling reaction such as Suzuki-Miyaura reaction between Intermediate A and the appropriate aryl boronic acid or its ester or an organotrifluoroborate (BF₃K) B provided Intermediate C. Removal of the protecting group using appropriate deprotection methods yielded final Compound D.

In some other embodiments, compounds described herein are prepared as described in Scheme B.

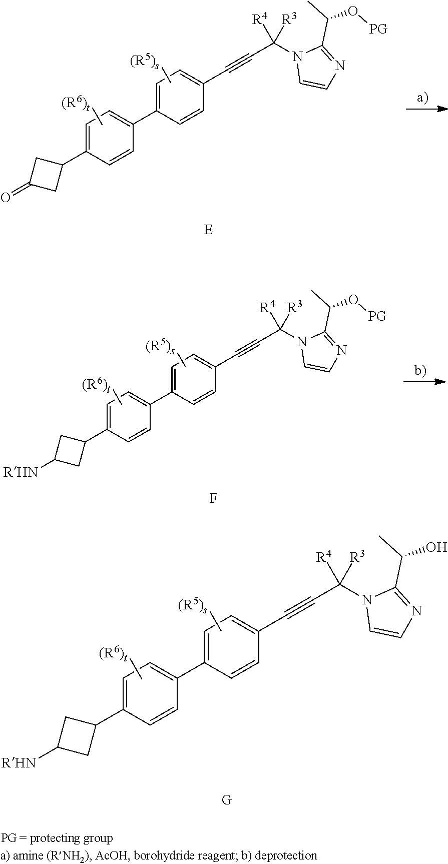

Ketone containing Intermediate E is reacted with an appropriate amine (R′″—NH₂) under appropriate reductive amination conditions (such as treatment with a borohydride reagent: for example, NaBH₄, NaCNBH₃, or NaB(OAc)₃H) to provide Intermediate F. Removal of the protecting group using appropriate deprotection methods yielded final Compound G.

In some embodiments, compounds are prepared as described in the Examples.

### Certain Terminology

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, C₁-Cₓ includes C₁-C₂, C₁-C₃ . . . C₁-Cₓ. By way of example only, a group designated as “C₁-C₆” indicates that there are one to six carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl. In some embodiments, an alkyl is methyl.

An “alkylene” group refers to a divalent alkyl radical. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. Typical alkylene groups include, but are not limited to, —CH₂—, —CH₂CH₂—, —CH₂CH₂CH₂—, —CH₂CH₂CH₂CH₂—, and the like. In some embodiments, an alkylene is —CH₂—.

An “alkoxy” group refers to a —O(alkyl) group, where alkyl is as defined herein.

The term “alkylamine” refers to the —N(alkyl)ₓH_{y} group, where x is 0 and y is 2, or where x is 1 and y is 1, or where x is 2 and y is 0.

An “hydroxyalkyl” refers to an alkyl in which one hydrogen atom is replaced by a hydroxyl. In some embodiments, a hydroxyalkyl is a C₁-C₄hydroxyalkyl. Typical hydroxyalkyl groups include, but are not limited to, —CH₂OH, —CH₂CH₂OH, —CH₂CH₂CH₂OH, —CH₂CH₂CH₂CH₂OH, and the like. In some embodiments, a hydroxyalkyl is —CH₂OH or —CH₂CH₂OH. In some embodiments, a hydroxyalkyl is —CH₂OH. In some embodiments, a hydroxyalkyl is —CH₂CH₂OH.

An “aminoalkyl” refers to an alkyl in which one hydrogen atom is replaced by an amino. In some embodiments, aminoalkyl is a C₁-C₄aminoalkyl. Typical aminoalkyl groups include, but are not limited to, —CH₂NH₂, —CH₂CH₂NH₂, —CH₂CH₂CH₂NH₂, —CH₂CH₂CH₂CH₂NH₂, and the like. In some embodiments, an amino alkyl is —CH₂NH₂ or —CH₂CH₂NH₂. In some embodiments, a hydroxyalkyl is —CH₂NH₂. In some embodiments, a hydroxyalkyl is —CH₂CH₂NH₂.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkynyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl. In some embodiments, a heteroalkyl is a C₁-C₆heteroalkyl where one or two atoms are independently selected from O, NH, and S.

The term “aromatic” refers to a planar ring having a delocalized π-electron system containing 4n+2 π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a phenyl, naphthyl, indanyl, indenyl, or tetrahydronaphthyl. In some embodiments, an aryl is a phenyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicyclo[1.1.1]pentyl. In some embodiments, a cycloalkyl is a C₃-C₆cycloalkyl. In some embodiments, a cycloalkyl is a C₃-C₄cycloalkyl. In some embodiments, a cycloalkyl is a cyclopropyl. In some embodiments, a cycloalkyl is a cyclobutyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a C₁-C₆fluoroalkyl. In some embodiments, a fluoroalkyl is —CF₃.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Monocyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 0-2 N atoms in the ring. In some embodiments, a heterocycloalkyl contains 0-2 N atoms, 0-2 O atoms and 0-1 S atoms in the ring. The heterocycloalkyl groups are optionally substituted with one or two oxo (═O) moieties.

The term “bond” or“single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

In some embodiments, each substituted alkyl, substituted fluoroalkyl, substituted heteroalkyl, substituted carbocycle, and substituted heterocycle is substituted with one or more R^{s} groups independently selected from the group consisting of halogen, C₁-C₆alkyl, monocyclic carbocycle, monocyclic heterocycle, —CN, —OR²¹, —CO₂R²¹, —C(═O)N(R²¹)₂, —N(R²¹)₂, —NR²¹C(═O)R²², —SR²¹, —S(═O)R²², —SO₂R²², and —SO₂N(R²¹)₂; each R²¹ is independently selected from hydrogen, C₁-C₆alkyl, C₁-C₆fluoroalkyl, C₁-C₆heteroalkyl, C₃-C₆cycloalkyl, C₂-C₆heterocycloalkyl, phenyl, benzyl, 5-membered heteroaryl and 6-membered heteroaryl; or two R²¹ groups are taken together with the N atom to which they are attached to form a N-containing heterocycle; each R²² is independently selected from C₁-C₆alkyl, C₁-C₆fluoroalkyl, C₁-C₆heteroalkyl, C₃-C₆cycloalkyl, C₂-C₆heterocycloalkyl, phenyl, benzyl, 5-membered heteroaryl and 6-membered heteroaryl.

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an antagonist. In some embodiments, a modulator is an inhibitor.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound of Formula (I), or a pharmaceutically acceptable salt thereof, and a co-agent, are both administered to a patient simultaneously in the form of a single entity or dosage. The term “non-fixed combination” means that the active ingredients, e.g. a compound of Formula (I), or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “article of manufacture” and “kit” are used as synonyms.

The term “subject” or “patient” encompasses mammals. Examples of mammals include, but are not limited to, any member of the Mammalian class: humans, non -human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development or progression of the disease or condition, relieving the disease or condition, causing regression of the disease or condition, relieving a secondary condition caused by the disease or condition, or stopping the symptoms of the disease or condition either prophylactically and/or therapeutically.

### Pharmaceutical Compositions

In certain embodiments, the heterocyclic LpxC inhibitory compound as described herein is administered as a pure chemical. In other embodiments, the heterocyclic LpxC inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one heterocyclic LpxC inhibitory compound as described herein, or a stereoisomer, pharmaceutically acceptable salt, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition.

Some embodiments provide a pharmaceutical composition comprising a compound of Formulas (I), (II), (III), (IV), (V), (VI), (VII), (VIII), (IX), (X), (XI), or (XII), or a pharmaceutically acceptable salt thereof, and a pharmaceutically acceptable excipient.

In certain embodiments, the heterocyclic LpxC inhibitory compound as described by Formulas (I), (II), (III), (IV), (V), (VI), (VII), (VIII), (IX), (X), (XI), or (XII) is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

The dose of the composition comprising at least one heterocyclic LpxC inhibitory compound as described herein differ, depending upon the patient's condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome), or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Combination Treatments

In certain instances, it is appropriate to administer at least one compound of Formulas (I), (II), (III), (IV), (V), (VI), (VII), (VIII), (IX), (X), (XI), or (XII), or a pharmaceutically acceptable salt thereof, in combination with on e or more other therapeutic agents.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, a compound of Formulas (I), (II), (III), (IV), (V), (VI), (VII), (VIII), (IX), (X), (XI), or (XII), or a pharmaceutically acceptable salt thereof, is co-administered with a second therapeutic agent, wherein the compound of Formulas (I), (II), (III), (IV), (V), (VI), (VII), (VIII), (IX), (X), (XI), or (XII), or a pharmaceutically acceptable salt thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply be additive of the two therapeutic agents or the patient experiences a synergistic benefit.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds of Formulas (I), (II), (III), (IV), (V), (VI), (VII), (VIII), (IX), (X), (XI), or (XII), or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject.

Other embodiments and uses will be apparent to one skilled in the art in light of the present disclosures. The following examples are provided merely as illustrative of various embodiments and shall not be construed to limit the invention in any way.

### EXAMPLES

As used above, and throughout the description of the invention, the following abbreviations, unless otherwise indicated, shall be understood to have the following meanings:

### Abbreviations

ACN or MeCN: acetonitrile;
aq: aqueous;
Boc or BOC: tert-butoxycarbonyl;
DCM: dichloromethane;
DIAD: diisopropyl azodicarboxylate;
DMAP: 4-dimethylaminopyridine;
DMF: dimethylformamide;
DMP: Dess-Martin periodinane
DPPA: diphenylphosphoryl azide;
d.r.: diastereomic ratio
Eq. or equiv: equivalents;
EtOAc: ethyl acetate;
g: grams
h or hr(s): hour(s);
HPLC: high-performance liquid chromatography;
LC-MS, LC MS, or LCMS: liquid chromatography-mass spectrometry;
LDA: lithium diisopropylamide;
M: molar;
MeOH: methanol;
mg: milligrams;
min: minute;
mL: milliliter;
mmol: millimole;
MsCl: methanesulfonyl (mesyl) chloride;
MTBE: methyl tert-butyl ether;
N: normal;
NBS: N-bromosuccinimide;
NMR: nuclear magnetic resonance;
Pet ether: petroleum ether;
PPTS: pyridinium p-toluenesulfonate;
p-TSA: para-toluenesuflonic acid
rt: room temperature;
SFC: supercritical fluid chromatography;
TEA: triethylamine (or Et₃N);
TFA: trifluoroacetic acid;
THF: tetrahydrofuran;
THP: tetrahydropyran;
TLC: thin layer chromatography;
TsCl: para-toluenesulfonyl (tosyl) chloride.

The following examples are provided for illustrative purposes only and not to limit the scope of the claims provided herein.

### I. Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted. Spectra are given in ppm (δ) and coupling constants, J are reported in Hertz. For proton spectra the solvent peak was used as the reference peak.

### Example A1: Synthesis of Compound 1, Compound 2 & Compound 3

### Experimental Procedure for Compound 1

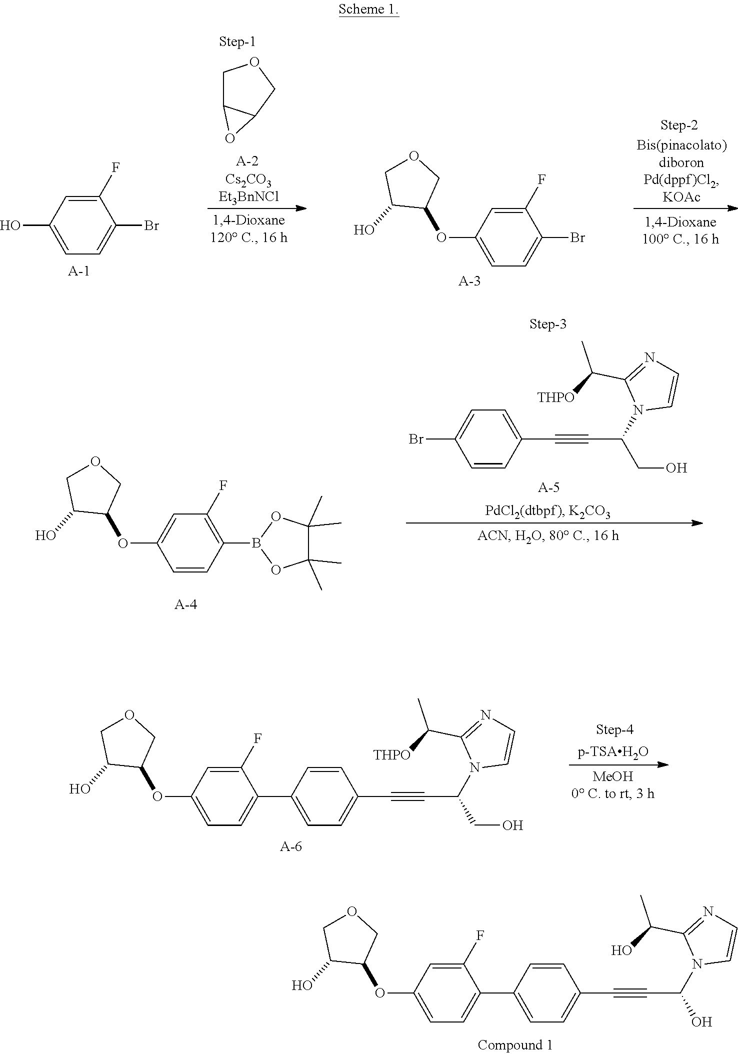

### Step-1:

To a stirred solution of 4-bromo-3-fluorophenol (A-1, 2.5 g, 13.09 mmol) in 1,4-dioxane (30 mL), were added 3,4-epoxytetrahydrofuran (A-2, 1.12 g, 13.09 mmol), cesium carbonate (6.38 g, 19.63 mmol) and benzyltriethylammonium chloride (0.594 g, 2.62 mmol) room temperature. The reaction mixture was stirred at 120° C. for 16 h. The reaction mixture was cooled to room temperature, filtered through a Celite pad and the bed further washed with EtOAc (3×50 mL). The filtrate was concentrated under reduced pressure to afford a crude mass, which was purified by flash column chromatography (SiO₂, 100-200 mesh size; 26% EtOAc in n-hexane) to afford A-3 (1.8 g, 50%) as an off-white solid.

### Step-2:

To a stirred solution of A-3 (1.8 g, 6.50 mmol) in 1,4-dioxane (20 mL) were added potassium acetate (1.91 g, 19.49 mmol) and bis(pinacolato)diboron (2.47 g, 9.74 mmol) at room temperature. The reaction mixture was degassed using nitrogen for 10 min. To this reaction mixture, was added Pd(dppf)Cl₂ (0.47 g, 0.65 mmol) and degassing continued for 2 min. The reaction mixture was heated at 100° C. for 16 h. The reaction mixture was cooled to room temperature, diluted with EtOAc (50 mL) and filtered through a Celite pad and the bed further washed with EtOAc (80 mL). The filtrate was washed with water (80 mL), brine (40 mL), dried over anhydrous sodium sulphate, filtered and concentrated under reduced pressure to afford a crude mass, which was purified by flash column chromatography (SiO₂, 100-200 mesh size; 26% EtOAc in pet ether) to afford A-4 (1.8 g, 85%) as a pale-yellow gum.

### Step-3:

To a stirred solution of A-4 (603 mg, 1.86 mmol) and A-5 (600 mg, 1.43 mmol) in acetonitrile (10 mL) and water (10 mL), was added potassium carbonate (593 mg, 4.29 mmol). The reaction mixture was degassed for 15 min using nitrogen gas. To this reaction mixture was added Pd(dtbpf)Cl₂ (93 mg, 0.14 mmol) and heated at 80° C. for 16 h. After completion, the reaction mixture was cooled to room temperature, diluted with water (80 mL) and extracted with EtOAc (50 mL×2). The combined organic layer was washed with brine (20 mL), dried over anhydrous sodium sulphate, filtered and concentrated under reduced pressure to afford a crude mass, which was purified by flash column chromatography (SiO₂, 100-200 mesh size; 80% EtOAc in pet ether) to afford A-6 (0.2 g, 25%) as a brown gum. LCMS: Calculated for C₃₀H₃₃FN₂O₆ is 536.60, Observed: 537.3 [M+1]⁺.

### Step-4:

To a stirred solution of A-6 (200 mg, 0.37 mmol) in MeOH (5 mL), was added p-toluenesulfonic acid monohydrate (142 mg, 0.74 mmol) in portions at 0° C. The resulting reaction mixture was stirred at room temperature for 3 h. The volatiles were evaporated under reduced pressure. to afford crude compound (380 mg) as a brown gum. The reaction mixture was diluted with water and extracted with 10% methanol in dichloromethane (2×25 mL). The combined organic layer was washed with sodium bicarbonate solution (2×30 mL), dried over anhydrous sodium sulphate, filtered and concentrated to afford crude compound as a brown gum, which was purified by reversed phase prep HPLC (10 mM NH₄HCO₃ in water and acetonitrile) to afford Compound 1 as a white solid. Yield: 0.01 g (6%). LCMS: Calculated for C₂₅H₂₅FN₂O₅ is 452.482, Observed: 453.2 [M+1]⁺.

### Experimental Procedure for Compound 2 & Compound 3

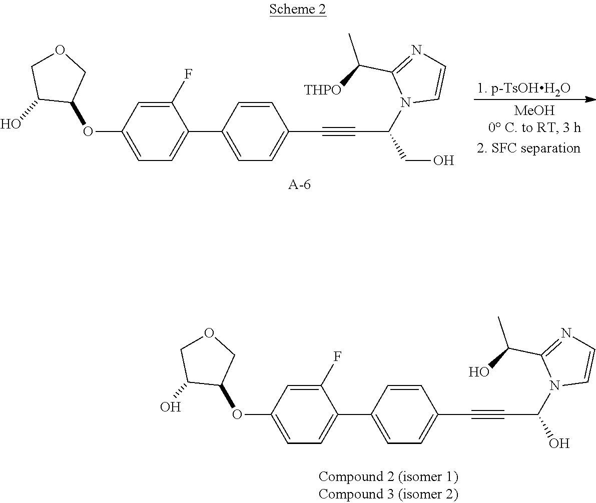

### Step-1:

To a stirred solution of A-6 (1.8 g, 3.35 mmol) in MeOH (18 mL) was added p-toluenesulfonic acid monohydrate (1.276 g, 6.71 mmol) in portions at 0° C. The resulting reaction mixture was stirred at room temperature for 3 h. The volatiles were evaporated under reduced pressure to afford crude residue, which was basified with sodium bicarbonate solution. This was extracted with 10% MeOH in DCM (2×80 mL). The combined organic layer was dried over anhydrous sodium sulfate, filtered and concentrated to afford crude which was purified by MPLC (manually packed cartridge; SiO₂ 100-200 mesh; 12% MeOH in DCM) to afford 600 mg of Compound 1 (mixture of diastereomers) as a brown solid. The isomers were separated by SFC (Column: LUX A1-(250*4.6) mm, 5 μm; Mobile phase: CO2:MeOH [70:30]). Collected fractions was evaporated under reduced pressure to afford Compound 2 (Peak-1 (first eluting), 100% (% ee=100), 114 mg, 7%): as a white solid and Compound 3 (Peak-2 (second eluting), 100% (% ee=100), 140 mg, 9%) as an off white solid. LCMS: Calculated for C25H25FN2O5 is 452.48, Observed: 453.2 [M+1]⁺.

### Example A2: Synthesis of Compound 4

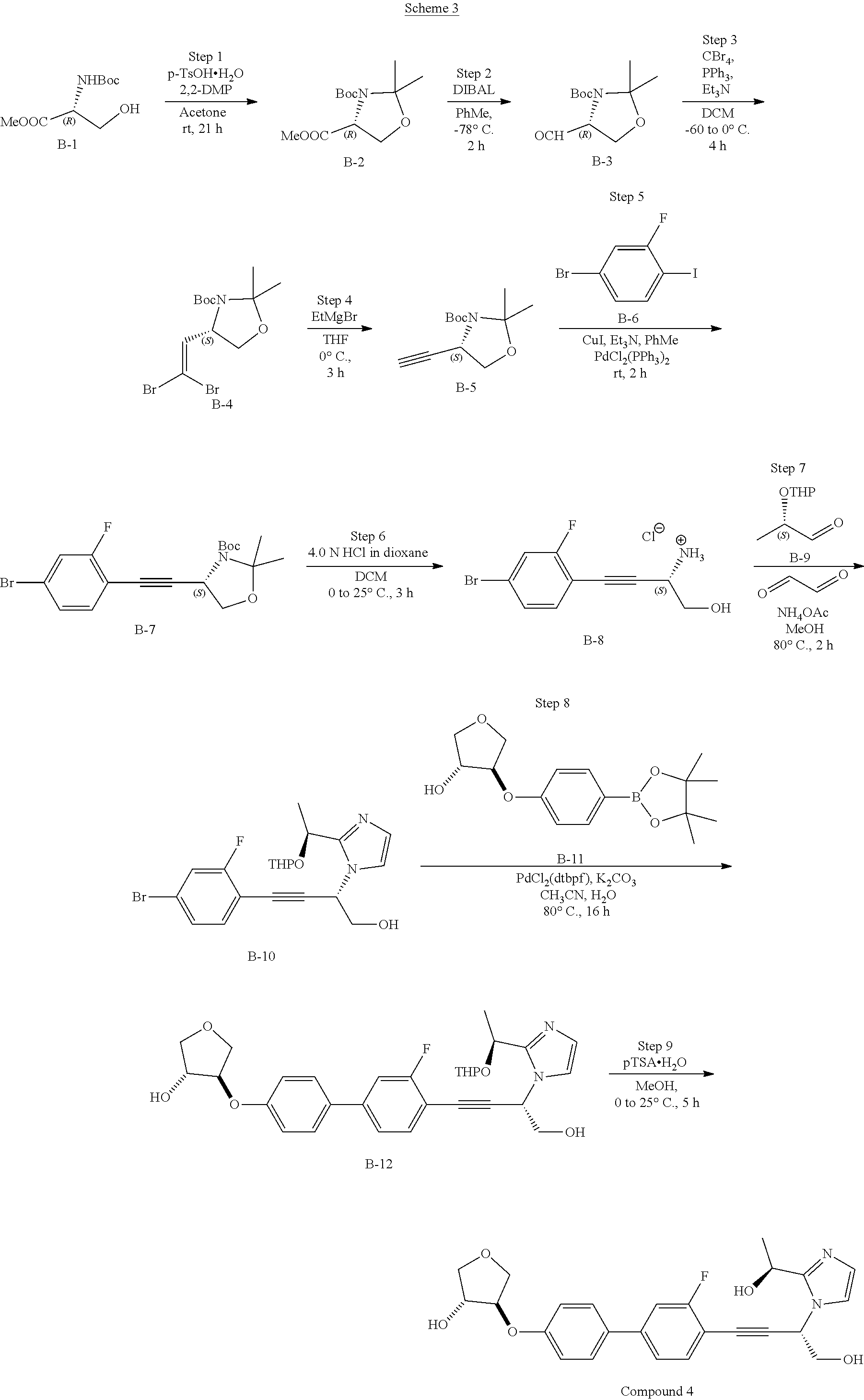

### Step 1:

To a stirred solution of methyl (tert-butoxycarbonyl)-D-serinate (B-1, 110 g, 502 mmol) in acetone (1500 mL), were added 2,2-Dimethoxy propane (313 g, 3010 mmol), and p-toluene sulfonic acid monohydrate (9.54 g, 50.2 mmol) at room temperature. The reaction mixture was stirred at room temperature for 21 h. The reaction mixture was concentrated un der reduced pressure. To the resulting residue, water (500 mL) was added and extracted with EtOAc (500 mL×3). The combined organic extract was washed with brine (200 mL), dried over anhydrous sodium sulphate, filtered and concentrated under reduced pressure to afford crude. The crude residue was purified by MPLC (manually packed cartridge SiO₂, 100-200 mesh size; 5-10% EtOAc in hexane) to obtain B-2 (71 g, 55%) as a colorless liquid.

### Step-2:

To a stirred solution of B-2 (71 g, 274 mmol) in toluene (700 mL), was added DIBAL-H (1.2 M in toluene; 342 mL, 411 mmol) at −78° C. After stirring for 3 h at −78° C., the reaction was quenched with MeOH (500 mL) at −78° C. and the resulting emulsion was slowly poured into an ice-cold solution of aq. HCl (1.5 N, 500 mL) and extracted with EtOAc (500 mL×3). The combined organic extract was washed with brine solution (200 mL), dried over anhydrous sodium sulphate, filtered and concentrated under reduced pressure to afford B-3 (62 g, 99%; crude product weight) as a colorless liquid.

### Step 3:

To a stirred solution of carbon tetrabromide (179 g, 541 mmol) in DCM (250 mL), was added a solution of triphenylphosphine (284 g, 1082 mmol) in DCM (250 mL) over a period of 20 min at −30° C. and stirred for 20 min at the same temperature. After 20 min, the resulting orange-red solution was cooled to −60° C., a solution of B-3 (62 g, 270 mmol) and trimethylamine (38.0 mL, 270 mmol) in DCM (250 mL) was added in dropwise manner over a period of 30 min and the reaction mixture gradually allowed to warm at 0° C. and stirred for 4 h. The reaction, as followed by TLC, showed complete consumption of starting material. The reaction was quenched by the addition of sat. sodium bicarbonate solution (300 mL) and extracted with DCM (500 mL×3). The combined organic extract was washed with water (300 mL), brine (500 mL), dried over anhydrous sodium sulphate, filtered and concentrated under reduced pressure to afford the crude residue. The crude residue was triturated with 20% EtOAc in hexanes (1000 mL×4) and the solids were filtered. The filtrate was concentrated under reduced pressure to afford a mass which was purified by MPLC (manually packed cartridge; SiO₂ 100-200 mesh; 5-7% EtOAc in pet ether) to afford the dibromoalkene B-4 (50 g, 48%) as an off-white solid.

### Step 4:

To a stirred solution of dibromoalkene B-4 (50 g, 130 mmol) in dry THE (400 mL), was added EtMgBr (2.0 M in THF; 130 mL, 260 mmol) at 0° C. over a period of 60 min and stirred for 3 h at the same temperature. After completion, the reaction was quenched by the addition of sat. NH₄Cl solution (400 mL) and extracted with EtOAc (500 mL×2). The combined organic extract was washed with brine (200 mL), dried over anhydrous sodium sulphate, filtered and concentrated under reduced pressure to afford a crude residue, which was purified by MPLC (manually packed cartridge; SiO₂ 230-400 mesh; 2% EtOAc in pet ether) to afford B-5 (21 g, 72%) as a colourless liquid.

### Step 5:

To a solution of B-5 (9 g, 39.90 mmol) in toluene (90 mL), were added 4-bromo-2-fluoro-1-iodobenzene (B-6, 14.42 g, 47.9 mmol) and triethylamine (16.70 mL, 120 mmol) at 25° C. and degassed with nitrogen for 5 min. To this reaction mixture, bis(triphenylphosphine)palladium(II) dichloride (0.561 g, 0.799 mmol) and Copper(I) iodide (0.456 g, 2.397 mmol) were added and the resulting reaction mixture stirred at room temperature for 2 h. The reaction mixture was diluted with EtOAc (250 mL) and the inorganic solids were filtered through Celite pad. The filtrate was washed with water (100 mL), brine (100 mL), dried over anhydrous sodium sulfate, filtered and concentrated under reduced pressure to afford crude residue. The crude residue was purified by MPLC (manually packed cartridge; SiO₂ 100-200 mesh size; 4% EtOAc in hexanes) to obtain B-7 (11 g, 69%) as white solid. LC-MS: Calculated for C₁₈H₂₁BrFNO₃ is 398.27, Observed: 300.0 [M-Boc+2]⁺ and 344.0 [M-56+2]⁺. SFC Purity: 97.63%.

### Step 6:

To a stirred solution of B-7 (11 g, 27.6 mmol) in DCM (110 mL), was added HCl (4.0 M in dioxane; 34.5 mL, 138 mmol) at 0° C. and the reaction mixture stirred at 25° C. for 3 h. The volatiles were removed under reduced pressure to afford the crude material. The crude material was triturated with 5% MeOH in DCM (200 mL), the solid precipitated filtered and dried under vacuum to afford B-8 (5 g, 61%) as an off-white solid. LC-MS: Calculated for C₁₀H₉BrFNO·ClH is 294.55; for the ammonium ion C₁₀H₁₀BrFNO is 259.10, Observed: 258.0 [M−1]⁺.

### Step 7:

To a stirred solution of B-8 (4.6 g, 15.62 mmol) in MeOH (60 mL), was added ammonium acetate (2.4 g, 31.2 mmol) at 25° C. and stirred for 10 min. To this reaction mixture, was added B-9 (9.88 g, 62.5 mmol;) followed by glyoxal (40% in water; 3.4 g, 23.43 mmol) after an interval of 10 min and the stirring continued. After 10 min stirring, the reaction mixture was heated at 80° C. for 2 h. The reaction mixture was cooled to room temperature and the volatiles removed under reduced pressure. To crude residue obtained was diluted with water (200 mL) and extracted with 5% MeOH in DCM (200 mL×2). The combined organic extract was washed with brine (100 mL), dried over anhydrous sodium sulphate, filtered and concentrated under reduced pressure. The resulting crude residue was purified by MPLC (manually packed cartridge; SiO₂ 230-400 mesh; 5% MeOH in DCM) to obtain B-10 (2.4 g, 35%) as brown gum. LC-MS: Calculated for C₂₀H₂₂BrFN₂O₃ is 437.30, Observed: 437.0 [M]⁺ and 439.0 [M+2]⁺.

### Step 8:

To a solution of B-10 (1 g, 2.28 mmol) in acetonitrile (10 mL) and water (10 mL), were added boronate ester B-11 (1.05 g, 3.43 mmol) and potassium carbonate (0.95 g, 6.86 mmol) and the mixture purged with nitrogen for 5 min. To this reaction mixture, PdCl2(dtbpf) (0.15 g, 0.23 mmol) was added and purging continued for 2 min. The reaction mixture was stirred at 80° C. for 16 h. The reaction was cooled to ambient temperature, diluted with water (100 mL) and extracted with 10% MeOH in DCM (100 mL×2). The combined organic extract was washed with brine (100 mL), dried over anhydrous sodium sulphate, filtered and concentrated under reduced pressure. The resulting crude was purified by MPLC (manually packed cartridge; SiO₂ 230-400 mesh size; 7% MeOH in DCM) to afford B-12 (0.4 g, 32%) as an off white solid. LC-MS: Calculated for C₃₀H₃₃FN₂O₆ is 536.6, Observed: 537.2 [M+1]⁺.

### Step 9:

To a stirred solution of B-12 (0.4 g, 0.745 mmol) in MeOH (10 mL), was added p-toluenesulfonic acid monohydrate (1.0 g, 5.26 mmol) at 0° C. The reaction mixture was warmed to 25° C. and stirred for 5 h. The volatiles were then evaporated under reduced pressure. The resulting residue was basified with 10% NaHCO₃ solution, extracted with 5% MeOH in DCM (50 mL×2). The layers were separated, the organic layer dried over anhydrous sodium sulphate, filtered and concentrated under reduced pressure. The resulting crude mass was purified by reversed phase prep HPLC (10 mM Ammonium bicarbonate in water and ACN) to afford desired product which was repurified by SFC (Column: 4-Ethylpyridine, Eluents: 0.5% isopropylamine in MeOH and CO₂) to afford Compound 4 (33 mg, 10%) as white solid. LC-MS: Calculated for C₂₅H₂₅FN₂O₅ is 452.48, Observed: 453.2 [M+1]⁺.

### Example A3: Synthesis of Compound 5

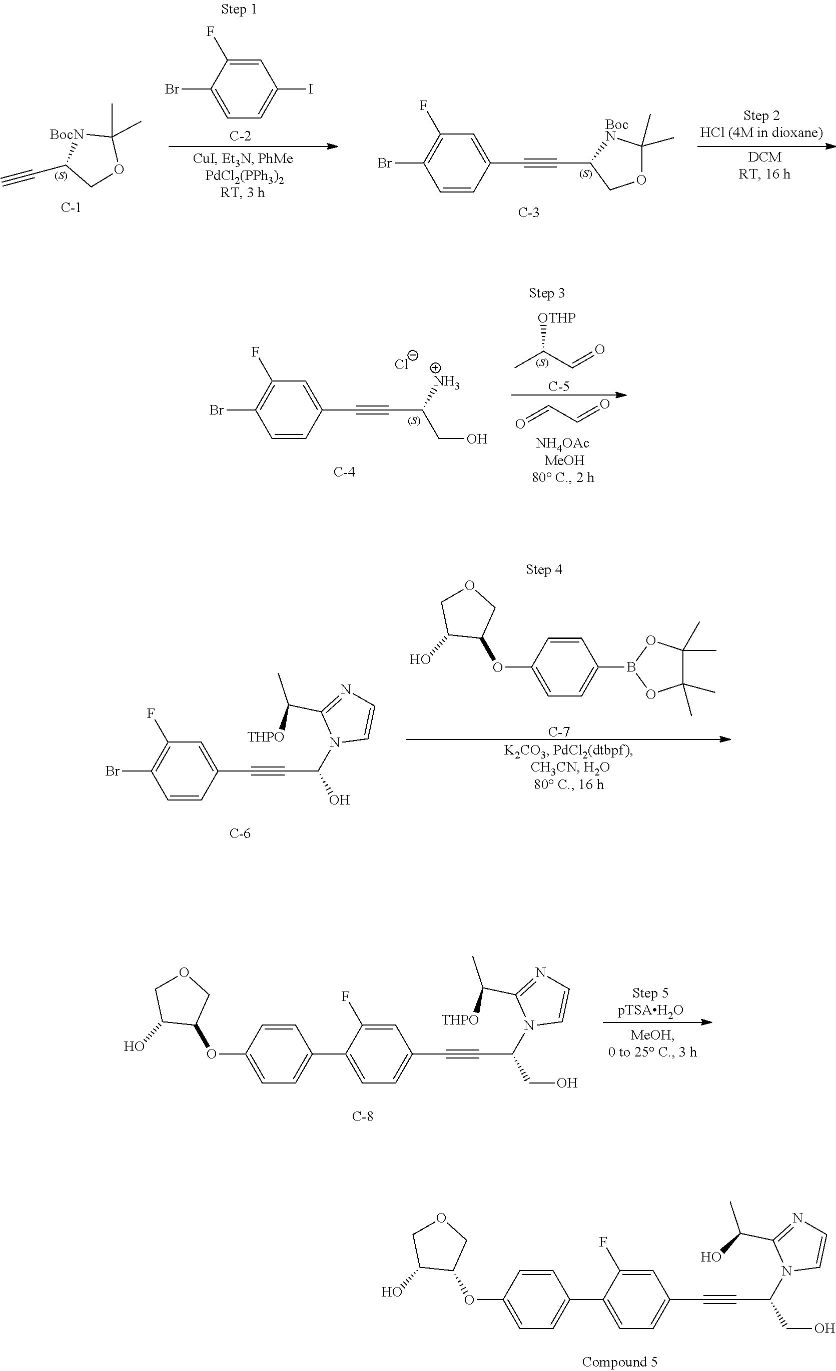

### Step 1:

To a stirred solution of C-1 (9 g, 39.90 mmol) in toluene (80 mL), were added 1-bromo-2-fluoro-4-iodobenzene (C-2, 14.42 g, 47.9 mmol) and triethylamine (16.70 mL, 120 mmol) at 25° C. and purged with nitrogen for 5 min. To this reaction mixture, bis(triphenylphosphine)-palladium(II) dichloride (0.561 g, 0.799 mmol) and Copper (I) iodide (0.456 g, 2.397 mmol) were added and the resulting reaction mixture stirred at room temperature for 3 h. The reaction was quenched with water and extracted with EtOAc (150 mL×2). The combined organic extract was washed with brine (100 mL), dried over anhydrous Na₂SO₄, filtered and concentrated under reduced pressure to afford crude residue. The crude residue was purified by MPLC (manually packed cartridge; SiO₂ 100-200 mesh size; 3% EtOAc in hexanes) to obtain C-3 (9.5 g, 59%) as an off-white solid. LC-MS: Calculated for C₁₈H₂₁BrFNO₃ is 398.27, Observed: 298.0 [M-Boc]⁺ and 300.0 [M-Boc+2]⁺. SFC Purity: 97.63%.

### Step 2:

To a stirred solution of C-3 (10 g, 27.6 mmol) in DCM (100 mL), HCl (4.0 M in dioxane; 31.4 mL, 126 mmol) was added at 0° C. The reaction mixture was stirred at 25° C. for 16 h. The volatiles present in the reaction mixture were evaporated under reduced pressure to afford crude residue. The crude material was triturated with 2% MeOH in DCM (100 mL), the solid precipitated was filtered and dried under vacuum to afford C-4 (4.9 g, 66%) as white solid. LC-MS: Calculated for the ammonium ion is 259.1, Observed: 258.0 [M]⁺ and 260.0 [M+2]⁺.

### Step 3:

To a stirred solution of C-4 (4.8 g, 18.6 mmol) in MeOH (50 mL), was added ammonium acetate (2.4 g, 31.2 mmol) at 25° C. and stirred for 10 min. To this reaction mixture, was added C-5 (11.77 g, 74.4 mmol) followed by glyoxal (40% in water; 3.19 mL, 27.9 mmol) after an interval of 10 min and the stirring continued. After 10 min stirring, the reaction mixture was heated at 80° C. for 2 h. The reaction mixture was cooled to room temperature and the volatiles were removed under reduced pressure to afford crude residue. To the crude residue, water (200 mL) was added and extracted with 5% MeOH in DCM (200 mL×2). The combined organic extract was washed with 10% NaHCO₃ solution (100 mL), dried over anhydrous Na₂SO₄, filtered and concentrated under reduced pressure. The resulting crude residue was purified by MPLC (manually packed cartridge; SiO₂ 230-400 mesh size; 80% EtOAc in hexanes) to obtain C-6 (3.5 g, 42%) as pale brown gum. LC-MS: Calculated for C₂₀H₂₂BrFN₂O₃ is 437.30, Observed: 437.0 [M]⁺ and 439.0 [M+2]⁺.

### Step 4:

To a solution of C-6 (1 g, 2.28 mmol) in acetonitrile (10 mL) and water (10 mL), were added boronate ester C-7 (1.05 g, 3.43 mmol) and potassium carbonate (0.95 g, 6.86 mmol) and the mixture purged with nitrogen for 5 min. To this reaction mixture, PdCl₂(dtbpf) (0.15 g, 0.23 mmol) was added and purging continued for 2 min. The reaction mixture was stirred at 80° C. for 16 h. The reaction was cooled to ambient temperature, water (100 mL) was added and extracted with 5% MeOH in DCM (100 mL×2). The combined organic extract was washed with brine (100 mL), dried over anhydrous Na₂SO₄, filtered and concentrated under reduced pressure. The resulting crude was purified by MPLC (manually packed cartridge; SiO2 230-400 mesh size; 7% MeOH in DCM) to afford C-8 (0.36 g, 29%) as pale brown solid. LC-MS: Calculated for C₃₀H₃₃FN₂O₆ is 536.6, Observed: 537.2 [M+1]⁺.

### Step 5:

To a stirred solution of C-8 (0.35 g, 0.652 mmol) in MeOH (10 mL), was added p-toluenesulfonic acid monohydrate (0.25 g, 1.30 mmol) at 0° C. The reaction mixture was warmed to 25° C. and stirred for 3 h. The volatiles were evaporated under reduced pressure, the resulting residue was basified with 10% NaHCO₃ solution and extracted with 5% MeOH in DCM (50 mL×2). The combined organic layer was dried over anhydrous sodium sulphate, filtered and concentrated under reduced pressure. The resulting crude mass was purified by reversed phase preparative HPLC (10 mM Ammonium bicarbonate in water and acetonitrile) to afford desired product which was repurified by SFC (Column: 4-Ethylpyridine, Eluents: 0.5% isopropylamine in MeOH and CO2) to afford Compound 5 as white solid. Yield=33 mg (10%). LC-MS: Calculated for C₂₅H₂₅FN₂O₅ is 452.48, Observed: 453.2 [M+1]⁺.

### Example A4: Synthesis of Compound 6, Compound 7, & Compound 8

Experimental procedure for Compound 6

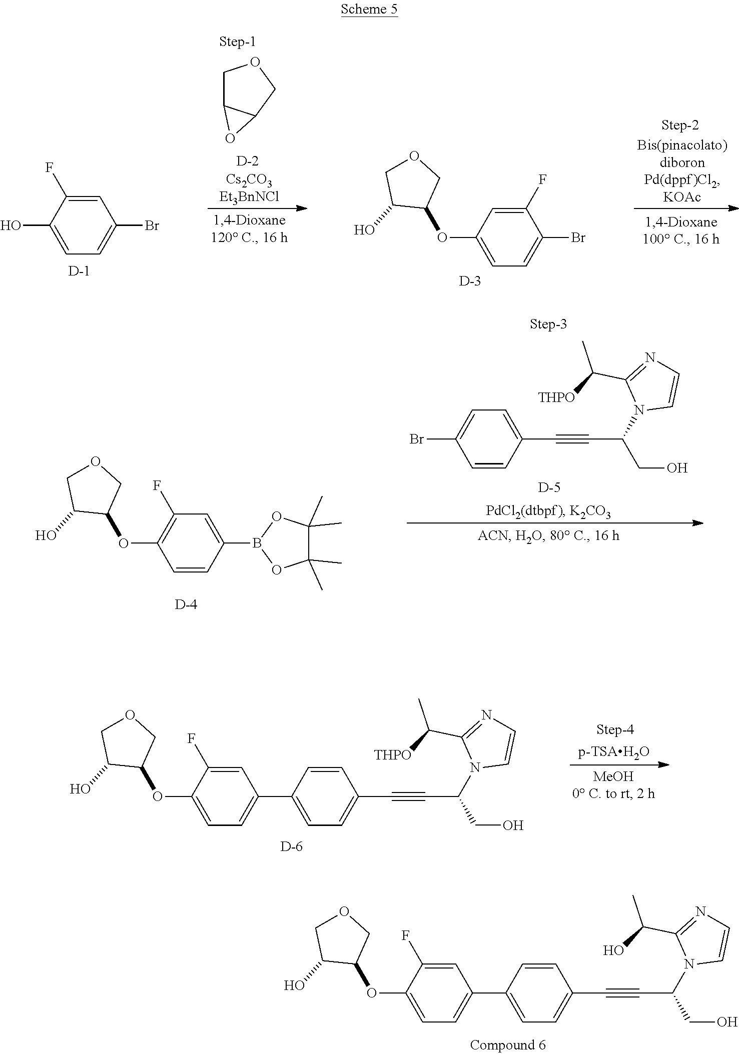

### Step-1:

To a stirred solution of 4-bromo-2-fluorophenol (D-1, 2.5 g, 13.09 mmol) in 1,4-dioxane (30 mL), were added 3,4-epoxytetrahydrofuran (D-2, 1.13 g, 13.09 mmol), cesium carbonate (6.38 g, 19.63 mmol) and benzyltriethylammonium chloride (0.6 g, 2.62 mmol) room temperature. The reaction mixture was stirred at 120° C. for 16 h. After completion, the reaction mixture was cooled to room temperature, filtered through a Celite pad and the bed further washed with EtOAc (3×50 mL). The filtrate was concentrated under reduced pressure and resulted pale-yellow gum was purified by flash column chromatography (SiO₂, 100-200 mesh size; 38% EtOAc in n-hexane) to afford D-3 (1.5 g, 41%) as an off-white solid.

### Step-2:

To a stirred solution of D-3 (1.5 g, 5.41 mmol) in 1,4-dioxane (20 mL), were added potassium acetate (1.59 g, 16.24 mmol) and bis(pinacolato)diboron (2.06 g, 8.12 mmol) at room temperature. The reaction mixture was degassed using nitrogen for 10 min. To this reaction mixture, was added Pd(dppf)Cl₂ (0.39 g, 0.54 mmol) and the degassing continued for 2 min. The reaction mixture was heated at 100° C. for 16 h. The reaction mixture was cooled to room temperature, diluted with EtOAc (50 mL) and filtered through a Celite bed and the bed further washed with EtOAc (80 mL). The filtrate was washed with water (80 mL) and brine (40 mL), dried over anhydrous sodium sulphate, filtered, and concentrated under reduced pressure to afford a crude residue. The resulting residue was purified by flash column chromatography (SiO₂, 100-200 mesh size; 29% EtOAc in pet ether) to afford D-4 (1.4 g, 73%) as a pale-yellow gum. LCMS: Calculated for C₁₆H₂₂BFO₅ is 324.155, Observed: 325.4 [M+1]⁺.

### Step-3:

To a stirred solution of D-4 (502 mg, 1.55 mmol) and D-5 (500 mg, 1.19 mmol; in acetonitrile (10 mL) and water (10 mL), was added potassium carbonate (494 mg, 3.58 mmol). The reaction mixture was degassed for 15 min using nitrogen gas. To this reaction mixture was added PdCl₂(dtbpf) (78 mg, 0.11 mmol). Then the reaction mixture was stirred at 80° C. for 16 h. The reaction mixture was cooled to room temperature, diluted with water (80 mL) and extracted with EtOAc (50 mL×2). The combined organic layer was washed with brine (20 mL), dried over anhydrous sodium sulphate, filtered and concentrated under reduced pressure. The resulting crude mass was purified by flash column chromatography (SiO₂, 100-200 mesh size; 80% EtOAc in pet ether) to afford D-6 (0.380 g, 50%) as a brown gum. LCMS: Calculated for C₃₀H₃₃FN₂O₆ is 536.60, Observed: 537.6 [M+1]⁺.

### Step-4:

To a stirred solution of D-6 (380 mg, 0.708 mmol) in MeOH (5 mL), was added p-toluenesulfonic acid monohydrate (269 mg, 1.416 mmol) in portions at 0° C. The resulting reaction mixture was stirred at room temperature for 2 h. The reaction mixture was evaporated under reduced pressure. To the residue added water and extracted with 10% MeOH in DCM (2×25 mL). The combined organic layer was washed with sodium bicarbonate solution (2×30 mL), dried over anhydrous sodium sulphate, filtered and concentrated. The resulting residue was purified by reversed phase prep HPLC (10 mM NH₄HCO₃ in H₂O and acetonitrile) to afford Compound 6 (0.02 g, 6%) as white solid. LCMS: Calculated for C₂₅H₂₅FN₂O₅ is 452.48, Observed: 453.2 [M+1]⁺.

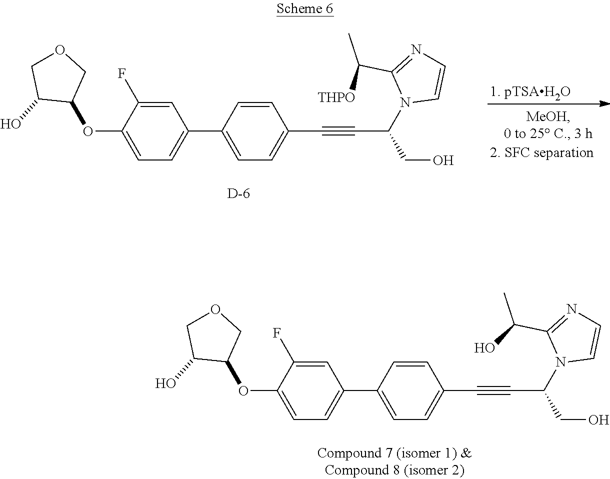

### Step-1:

To a stirred solution of D-6 (1.9 g, 3.54 mmol) in MeOH (19 mL), was added p-toluenesulfonic acid monohydrate (1.347 g, 7.08 mmol) in portions at 0° C. The resulting reaction mixture was stirred at room temperature for 3 h. The volatiles were evaporated under reduced pressure to afford crude compound. This was basified with sodium bicarbonate solution and extracted with 10% MeOH in DCM (2×80 mL). The combined organic layer was dried over anhydrous sodium sulfate, filtered and concentrated to afford crude, which was purified by MPLC (manually packed cartridge; SiO₂ 100-200 mesh; 12% MeOH in DCM) to 780 mg of Compound 6 (mixture of isomers) as a brown solid. The isomers of Compound 6 (mixture of isomers) were separated by SFC (Column: LUX A1-(250*4.6) mm, 5 μm; eluents: CO₂:0.5% IPAM in MeOH [60:40]). The collected fractions were evaporated under reduced pressure to afford Compound 7 (Peak-1 (first eluting)), (250 mg) and Compound 8 (Peak-2 (second eluting)), (158 mg) as an off-white solids. ¹H NMR of Compound 7 (250 mg) showed aliphatic impurities; it was further re-purified by reversed phase prep HPLC (10 mM ammonium bicarbonate in water and acetonitrile) Compound 7 (110 mg, (SFC % ee=100)) as an off-white solid and Compound 8 (158 mg, (SFC % ee=98.37)). LCMS: Calculated for C₂₅H₂₅FN₂O₅ is 452.48, Observed: 453.2 [M+1]⁺.

### Example A5: Synthesis of Compound 9, Compound 10, & Compound 11

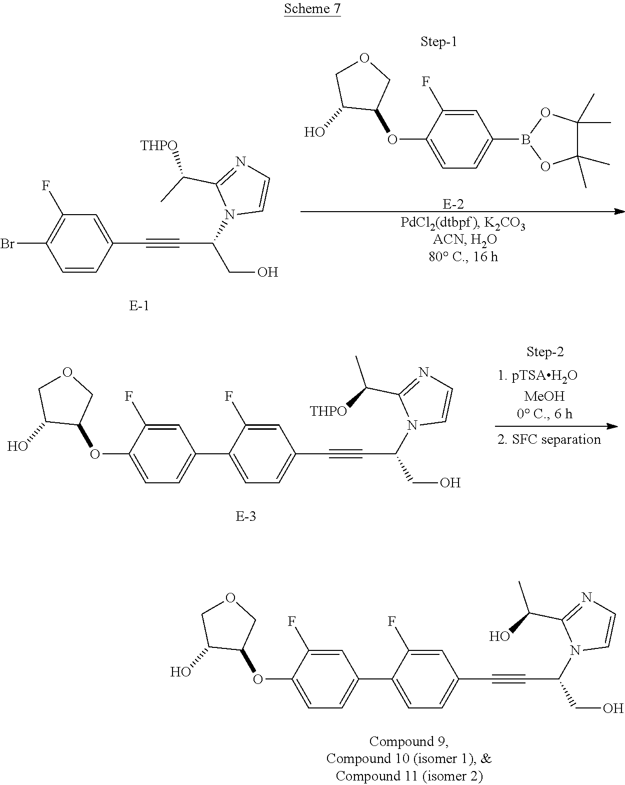

### Step 1:

To a solution of E-1 (4.0 g, 9.15 mmol) in acetonitrile (60 mL) and water (60 mL), were added boronate ester E-2 (2.96 g, 9.15 mmol) and potassium carbonate (3.79 g, 27.4 mmol) and the reaction mixture purged with nitrogen for 5 min. To this reaction mixture, PdCl₂(dtbpf) (0.596 g, 0.915 mmol) was added and the purging continued for 2 min. The reaction mixture was stirred at 80° C. for 16 h. The reaction was cooled to ambient temperature, water (100 mL) was added and extracted with 10% MeOH in DCM (200 mL×2). The combined organic extract was washed with brine (100 mL), dried over anhydrous Na₂SO₄, filtered and concentrated under reduced pressure. The resulting crude was purified by MPLC (manually packed cartridge; SiO₂ 100-200 mesh size; 3% MeOH in DCM) to afford E-3 as pale-brown solid. Yield=3 g (58%). LC-MS: Calculated for C₃₀H₃₂F₂N₂O₆ is 554.59, Observed: 555.2 [M+1]⁺.

### Step 2:

To a stirred solution of E-3 (3 g, 5.41 mmol) in MeOH (200 mL), p-toluene sulfonic acid monohydrate (0.931 g, 5.41 mmol) was added at 0° C. The reaction mixture was warmed to room temperature and stirred for 5 h. The volatiles were evaporated under reduced pressure. The resulting residue was basified with 10% NaHCO3 solution and extracted with 5% MeOH in DCM (150 mL×2) and dried over anhydrous sodium sulphate, filtered and concentrated under reduced pressure. The resulting crude was purified by MPLC (manually packed cartridge; SiO₂ 100-200 mesh size; 3% MeOH in DCM) to afford Compound 9 (mixture of diastereomers; racemic at tail) as a light brown solid. Yield: 1.2 g (56%). LC-MS: Calculated for C₂₅H₂₄F₂N₂O₅ is 470.473, Observed: 471.4 [M+1]⁺. The isomers of Compound 9 (mixture of diastereomers) were separated by SFC (Column: Lux A1 (250*20) mm, 5 μm; Eluents: CO₂:0.5% isopropylamine in MeOH [70:30]). After SFC separation, the fractions were concentrated under reduced pressure to afford Compound 10 (Peak-1 (first eluting)) (420 mg, SFC=82.5%) and Compound 11 (Peak-2 (second eluting)), (320 mg, SFC=97.9%). Both the isomers were re-purified by SFC. Compound 10 (mixture of diastereomers) was re-purified by SFC (Column: Lux A1-(250*20) mm, 5 μm; Eluents: CO₂:0.5% isopropylamine in MeOH [70:30]). The fractions were concentrated under reduced pressure and dried under vacuum. Yield of Compound 10: 250 mg (SFC purity=92.4%). Compound 11: (mixture of diastereomers) were re-purified by SFC (Column: Amylose A-(250*20) mm, 5 μm; Eluents: CO₂:0.5% isopropylamine in MeOH [70:30]). The fractions were concentrated under reduced pressure and dried under vacuum. Yield of Compound 11: 250 mg (SFC purity=99.8%).

### Example A6: Synthesis of Compound 12, Compound 13, & Compound 14

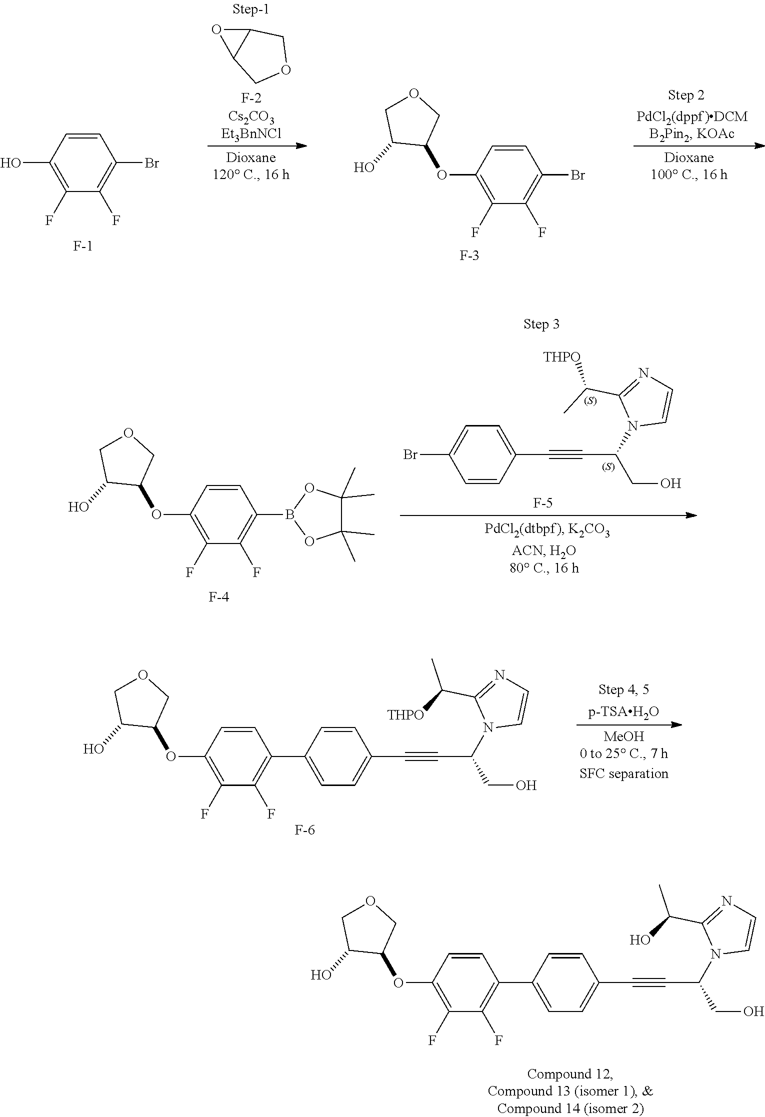

### Step 1:

To a stirred solution of 4-bromo-2,3-difluorophenol (F-1, 12.0 g, 57.4 mmol) in 1,4-dioxane (130 mL), were added 3,4-epoxytetrahydrofuran (F-2, 7.41 g, 86 mmol), Cs₂CO₃ (37.4 g, 115 mmol) and benzyltriethylammonium chloride (2.61 g, 11.48 mmol) at room temperature. The reaction mixture was heated at 120° C. for 16 h. After completion of reaction, the inorganic solids were filtered through a Celite pad and washed with DCM (150 mL×2). The filtrate was concentrated under reduced pressure. The resulting crude residue was purified by MPLC (manually packed cartridge; SiO₂ 100-200 mesh; 38% EtOAc in hexanes) to afford F-3 (5.0 g, (25%) as pale-yellow gum.

### Step 2:

To a stirred solution of F-3 (7.0 g, 23.72 mmol) in 1,4-dioxane (70 mL), were added bis(pinacolato)diboron (9.04 g, 35.6 mmol) and potassium acetate (6.98 g, 71.2 mmol) at room temperature. The reaction mixture was degassed using nitrogen for 10 min. To this reaction mixture, PdCl₂(dppf)·CH₂Cl₂ adduct (1.736 g, 2.372 mmol) was added and degassing continued for 2 min. The reaction mixture was stirred at 100° C. for 16 h. After completion of reaction, the inorganic solids were filtered off through celite pad and washed with DCM (150 mL). The filtrate was washed with water (150 mL), brine (50 mL), dried over anhydrous sodium sulphate, filtered and concentrated under reduced pressure. The resulting crude residue was purified by MPLC (manually packed cartridge; SiO₂ 100-200 mesh; 40% EtOAc in hexanes) to afford F-4 (6.8 g, 75%) as pale-yellow gum.

### Step 3:

To a solution of F-5 (4 g, 9.54 mmol) in acetonitrile (60 mL) and water (60 mL), boronate ester F-4 (4.9 g, 14.31 mmol) and potassium carbonate (3.96 g, 28.6 mmol) were added. The reaction mixture was purged with nitrogen for 15 min. To this reaction mixture, PdCl₂(dtbpf) (0.622 g, 0.954 mmol) was added and purging continued for 5 min. The reaction mixture was stirred at 80° C. for 16 h. The reaction was cooled to ambient temperature, water (100 mL) was added and extracted with 10% MeOH in DCM (200 mL×2). The combined organic extract was washed with brine (100 mL), dried over anhydrous Na₂SO₄, filtered and concentrated under reduced pressure. The resulting crude was purified by MPLC (manually packed cartridge; SiO₂ 230-400 mesh size; 7% MeOH in DCM) to afford F-6 (3.5 g, 56%) as pale-brown solid.

LC-MS: Calculated for C₃₀H₃₂F₂N₂O₆ is 554.59, Observed: 555.2 [M+1]⁺

### Step 4:

To a stirred solution of F-6 (3.0 g, 5.41 mmol) in MeOH (170 mL), p-toluenesulfonic acid monohydrate (2.058 g, 10.82 mmol) was added at 0° C. The reaction mixture was warmed to 25° C. and stirred for 7 h. The volatiles were evaporated under reduced pressure, the resulting residue was basified with 10% NaHCO3 solution (50 mL) and extracted with 10% MeOH in DCM (200 mL×2). The combined organic extract was washed with brine (50 mL), dried over anhydrous Na₂SO₄, filtered and concentrated under reduced pressure. The resulting crude mass was purified by MPLC (manually packed, cartridge; SiO₂ 230-400 mesh size; 10% MeOH in DCM) to afford Compound 12 (1.1 g, 44%) as a pale-brown solid. LC-MS: Calculated for C₂₅H₂₄F₂N₂O₅ is 470.47, Observed: 471.2 [M+1]⁺. The isomers of Compound 12 were separated by SFC (Column: LUX-A1 (250*30) mm, 5 μm; Eluents: CO₂:0.5% isopropylamine in MeOH [80:20]). After SFC separation, the fractions were concentrated under reduced pressure to afford Compound 13 ((Peak-1 (first eluting), t_{R}=3.33 min) and Compound 14 Isomer 2 ((Peak-2 (second eluting), t_{R}=5.13 min). Yield=Compound 13 0.3 g (% ee=100) and Compound 14 0.3 g (% ee=100)

### Example A7: Synthesis of Compound 15, Compound 16, & Compound 17

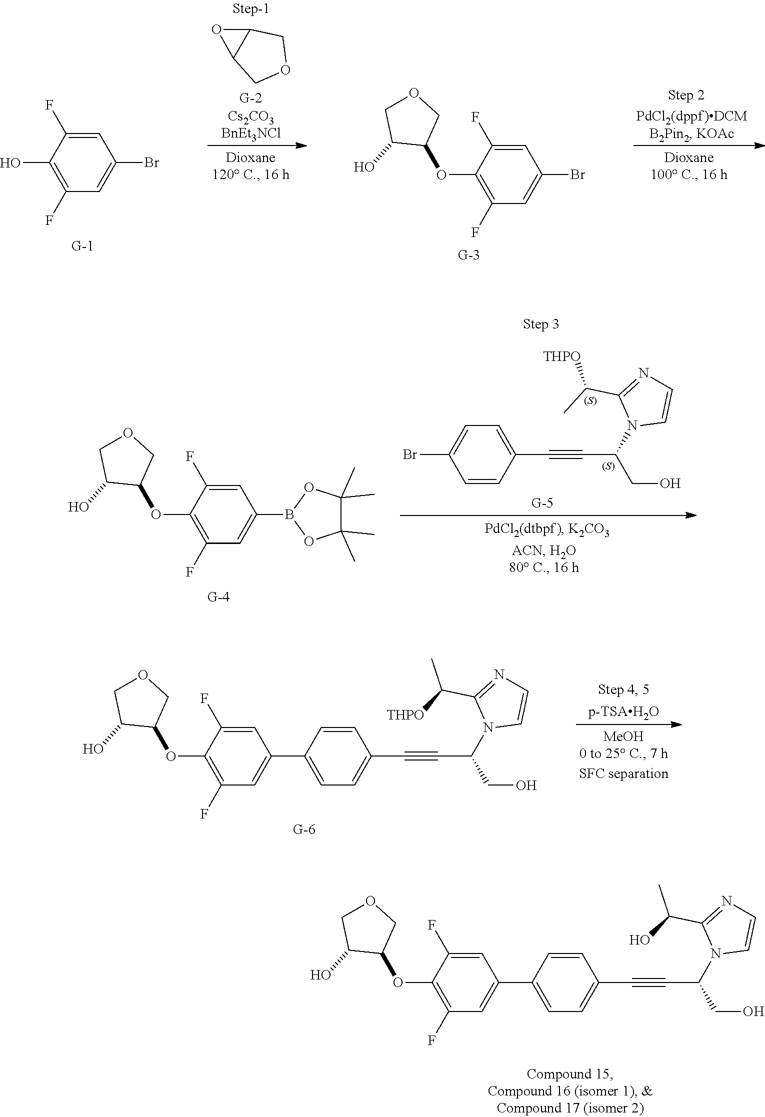

### Step 1:

To a stirred solution of 4-bromo-2,6-difluorophenol (G-1, 12.0 g, 57.4 mmol) in 1,4-dioxane (130 mL), were added 3,4-epoxytetrahydrofuran (G-2, 7.41 g, 86 mmol), Cs₂CO₃ (37.4 g, 115 mmol) and benzyltriethylammonium chloride (2.61 g, 11.48 mmol) at room temperature. The reaction mixture was heated at 120° C. for 16 h. After completion of reaction, the inorganic solids were filtered through a Celite pad and washed with DCM (150 mL×2). The filtrate was concentrated under reduced pressure. The resulting crude residue was purified by MPLC (manually packed cartridge; SiO₂ 100-200 mesh; 38% EtOAc in hexanes) to afford G-3 (5.0 g, 25%) as pale-yellow gum.

### Step 2:

To a stirred solution of G-3 (7.0 g, 23.72 mmol) in 1,4-dioxane (70 mL), were added bis(pinacolato)diboron (9.04 g, 35.6 mmol) and potassium acetate (6.98 g, 71.2 mmol) at room temperature. The reaction mixture was degassed using nitrogen for 10 min. To this reaction mixture, PdCl₂(dppf)·CH₂Cl₂ adduct (1.736 g, 2.372 mmol) was added and degassing continued for 2 min. The reaction mixture was stirred at 100° C. for 16 h. After completion of reaction, the inorganic solids were filtered off through celite pad and washed with DCM (150 mL). The filtrate was washed with water (150 mL), brine (50 mL), dried over anhydrous sodium sulphate, filtered and concentrated under reduced pressure. The resulting crude residue was purified by MPLC (manually packed cartridge; SiO₂ 100-200 mesh; 40% EtOAc in hexanes) to afford G-4 (6.8 g, 75%) as pale-yellow gum.

### Step 3:

To a solution of G-5 (4 g, 9.54 mmol) in acetonitrile (60 mL) and water (60 mL), boronate ester G-4 (4.9 g, 14.31 mmol) and potassium carbonate (3.96 g, 28.6 mmol) were added. The reaction mixture was purged with nitrogen for 15 min. To this reaction mixture, PdCl₂(dtbpf) (0.622 g, 0.954 mmol) was added and purging continued for 5 min. The reaction mixture was stirred at 80° C. for 16 h. The reaction was cooled to ambient temperature, water (100 mL) was added and extracted with 10% MeOH in DCM (200 mL×2). The combined organic extract was washed with brine (100 mL), dried over anhydrous Na₂SO₄, filtered and concentrated under reduced pressure. The resulting crude was purified by MPLC (manually packed cartridge; SiO2 230-400 mesh size; 7% MeOH in DCM) to afford G-6 (3.5 g, 56%) as pale-brown solid.

LC-MS: Calculated for C₃₀H₃₂F₂N₂O₆ is 554.59, Observed: 555.2 [M+1]⁺

### Step 4:

To a stirred solution of G-6 (3.0 g, 5.41 mmol) in MeOH (170 mL), p-toluenesulfonic acid monohydrate (2.058 g, 10.82 mmol) was added at 0° C. The reaction mixture was warmed to 25° C. and stirred for 7 h. The volatiles were evaporated under reduced pressure, the resulting residue was basified with 10% NaHCO₃ solution (50 mL) and extracted with 10% MeOH in DCM (200 mL×2). The combined organic extract was washed with brine (50 mL), dried over anhydrous Na₂SO₄, filtered and concentrated under reduced pressure. The resulting crude mass was purified by MPLC (manually packed, cartridge; SiO₂ 230-400 mesh size; 10% MeOH in DCM) to afford Compound 15 (1.1 g, 44%) as a pale-brown solid. LC-MS: Calculated for C₂₅H₂₄F₂N₂O₅ is 470.47, Observed: 471.2 [M+1]⁺. The isomers of Compound 15 were separated by SFC (Column: LUX-A1 (250*30) mm, 5 μm; Eluents: CO₂: 0.5% isopropylamine in MeOH [80:20]). After SFC separation, the fractions were concentrated under reduced pressure to afford Compound 16 (Peak-1 (first eluting)), (0.3 g, (% ee=100)) and Compound 17 (Peak-2 (second eluting)), (0.3 g, % ee=100)).

### Example A8: Synthesis of Compounds 45 and 46

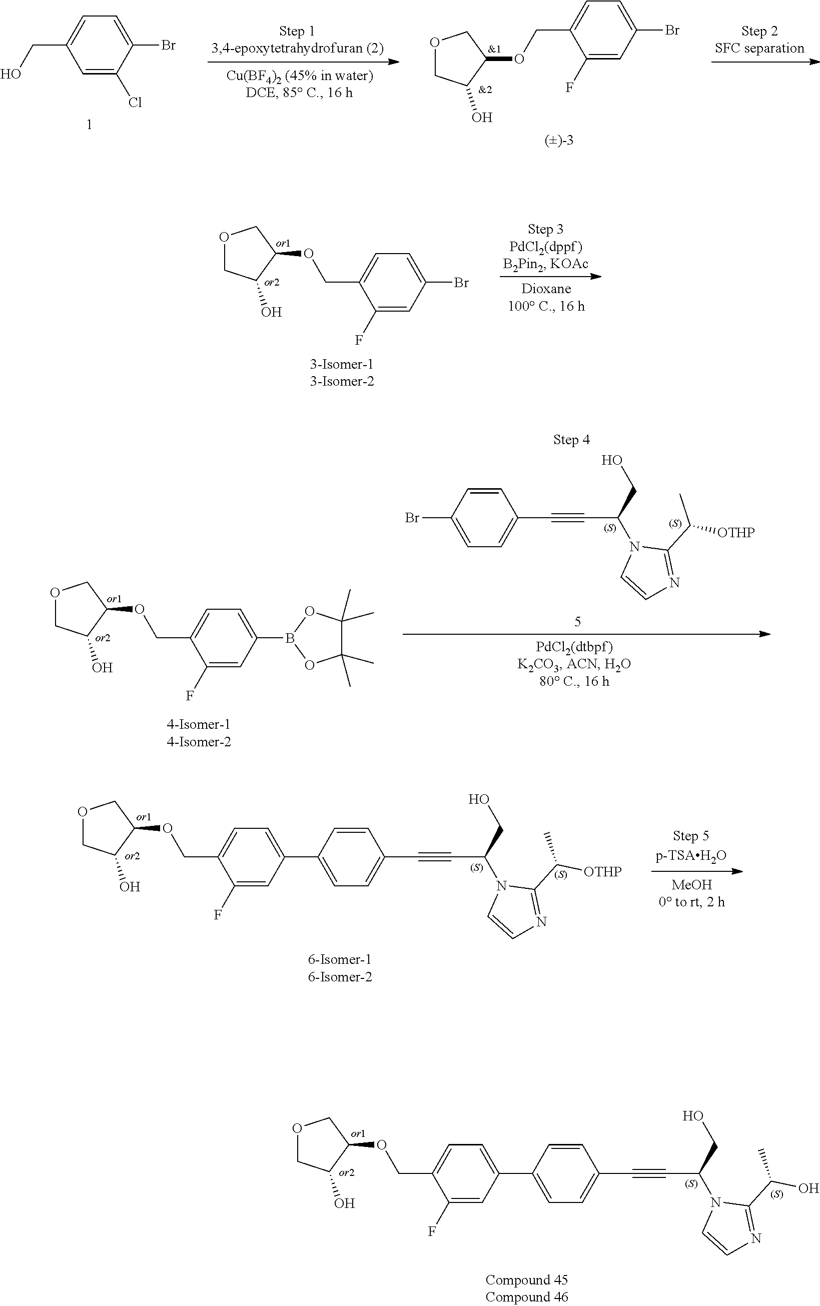

### Step 1:

To a stirred solution of 3,4-epoxytetrahydrofuran (2, 11.37 mL, 159 mmol) in DCE (150 mL), were added 4-Bromo-2-fluorobenzyl alcohol (1, 13 g, 63.4 mmol) and Copper (II) tetrafluoroborate (45% in water, 4.45 mL, 12.68 mmol) at room temperature, and the reaction mixture stirred at 85° C. for 16 h. The reaction mixture was quenched with water (500 mL) and extracted with DCM (300 mL×2). The combined organic layer was washed with brine (50 mL), dried over anhydrous Na₂SO₄, filtered and concentrated under reduced pressure. The crude residue was purified by using MPLC (manually packed cartridge, SiO₂ 230-400 mesh; 50% EtOAc in hexanes) to afford (±)-3 as a pale-yellow liquid. Yield: 3 g (15%).

### Step 2:

The enantiomers (±)-3 (11 g) was separated by using SFC (Column: CHIRALPAK AS-H (250×30) mm, 5 μm; Eluents: CO₂ and 0.5% isopropyl amine in IPA [80:15]) to get 3-Isomer-1 (tR=3.09 min) as an off-white solid and 3-Isomer-2 (tR=4.17 min) as off -white solids. Yield: 3-Isomer-1=3.2 g and 3-Isomer-2=3.2 g. SFC: 3-Isomer-1: 100% (ee=100%) and 3-Isomer-2: 98.92% (ee=98.1%). Both the isomers (3-Isomer-1 and 3-Isomer-2) were taken independently for further conversion. The synthesis of Compound 45 was carried out using 3-Isomer-1.

### Step 3:

To a stirred solution of 3-Isomer-1 (3.3 g, 11.34 mmol) in 1,4-dioxane (40 mL), were added potassium acetate (3.34 g, 34.0 mmol) and bis(pinacolato)diboron (4.32 g, 17.00 mmol) at 25° C., and the reaction mixture purged with nitrogen for 5 min. To this reaction mixture, PdCl2(dppf) (0.829 g, 1.134 mmol) was added and stirred at 100° C. for 16 h. The reaction mixture was cooled to room temperature, quenched with water (100 mL) and extracted with EtOAc (2×150 mL). The combined organic extract was washed with brine (50 mL), dried over anhydrous Na2SO4, filtered and concentrated under reduced pressure. The resulting crude residue was purified by using MPLC (manually packed SiO₂ cartridge, 100-200 mesh size; 30% EtOAc in hexanes) to obtain 4-Isomer-1 as a brown solid. Yield: 2.8 g (62%).

### Step 4:

To a stirred solution of 4-Isomer-1 (0.968 g, 2.86 mmol) in mixture of water (10 mL) and acetonitrile (10 mL), were added 5 (1 g, 2.385 mmol) and potassium carbonate (0.989 g, 7.15 mmol) at room temperature. The reaction mixture purged with nitrogen for 5 min. To this reaction mixture, PdCl2(dtbpf) (0.078 g, 0.119 mmol) was added and stirred at 80° C. for 16 h. The reaction mixture was quenched with water (30 mL) and extracted with 10% MeOH in DCM (20 mL×2). The combined organic layer was dried over anhydrous Na₂SO₄, filtered and concentrated under reduced pressure. The crude residue, thus obtained, was purified by using MPLC (manually packed SiO₂ cartridge, 230-400 mesh size; 5% MeOH in DCM) to obtain 6-Isomer-1 as a brown solid. Yield: 0.62 g (45%). LCMS: Calculated for C31H35FN2O6 is 550.63, Observed: 551.2 [M+1]⁺.

### Step 5:

To a stirred solution of 6-Isomer-1 (0.6 g, 1.090 mmol) in MeOH (30 mL), was added p-toluenesulfonic acid monohydrate (0.622 g, 3.27 mmol) at 0° C., and the reaction mixture stirred at room temperature for 2 h. The reaction mixture was basified with sat. NaHCO3 solution (30 mL) at 0° C. The aqueous layer was extracted with 10% MeOH in DCM (20 mL×2). The combined organic layer was washed with brine (10 mL), filtered, and concentrated under reduced pressure. The crude residue, thus obtained, was purified by reverse phase column chromatography (Column: Redisep C18 SiO2; Eluents: 10 mM ammonium bicarbonate in water and ACN) to afford Compound 45 as a white solid. Yield: 220 mg (43%). LCMS: Calculated for C26H27FN2O5 is 466.5; Observed: 467.2 [M+1]⁺. 1H-NMR (400 MHz, DMSO-d6): δ 7.76 (d, J=8.40 Hz, 2H), 7.59-7.51 (m, 5H), 7.36 (d, J=1.20 Hz, 1H), 6.84 (d, J=1.20 Hz, 1H), 5.70 (t, J=5.60 Hz, 1H), 5.54 (t, J=6.00 Hz, 1H, exchanges with D2O), 5.38 (d, J=5.60 Hz, 1H, exchanges with D2O), 5.18 (d, J=4.00 Hz, 1H, exchanges with D2O), 4.97-4.92 (m, 1H), 4.65 (d, J=12.00 Hz, 1H), 4.60 (d, J=12.00 Hz, 1H), 4.20 (t, J=4.00 Hz, 1H), 3.93 (d, J=4.40 Hz, 1H), 3.89-3.79 (m, 4H), 3.76 (dd, J=1.20, 9.60 Hz, 1H), 3.53 (dd, J=2.00, 9.20 Hz, 1H), 1.51 (d, J=6.40 Hz, 3H). 19F-NMR (376 MHz, DMSO-d6): δ −118.21-118.25 (m). SFC: 96.6% (de=100%); tR=5.56 min (Column: I Cellulose-Z; Eluents: 0.5% isopropyl amine in MeOH and CO2). Unknown stereochemistry at tail; single isomer with SFC purity=96.6%.

Compound 46 was synthesized in an identical fashion starting with 3-Isomer-2 by using the steps (Step 3 to step 5) detailed for Compound 45. LCMS: Calculated for C26H27FN2O5 is 466.5; Observed: 467.4 [M+1]⁺. 1H-NMR (400 MHz, DMSO-d6): δ 7.76 (d, J=8.40 Hz, 2H), 7.57-7.53 (m, 5H), 7.36 (d, J 1.20 Hz, 1H), 6.84 (d, J=0.80 Hz, 1H), 5.70 (t, J=6.00 Hz, 1H), 5.54 (t, J=5.60 Hz, 1H, exchanges with D2O), 5.37 (d, J=5.60 Hz, 1H, exchanges with D2O), 5.18 (d, J=4.00 Hz, 1H, exchanges with D2O), 4.97-4.92 (m, 1H), 4.65 (d, J=12.40 Hz, 1H), 4.59 (d, J 12.40 Hz, 1H), 4.20 (br s, 1H), 3.93 (d, J=4.00 Hz, 1H), 3.80-3.89 (m, 4H), 3.71 (d, J=9.60 Hz, 1H), 3.53 (dd, J=1.60, 9.20 Hz, 1H), 1.51 (d, J=6.40 Hz, 3H). 19F-NMR (376 MHz, DMSO-d6): δ −118.209-118.259 (m, 1F). SFC: 98.0%; tR=6.24 min (Column: I Cellulose-Z; Eluents: 0.5% isopropyl amine in MeOH and CO2). Unknown stereochemistry at tail; single isomer with SFC purity=98.0%.

### Example A9: Synthesis of Compounds 47 and 48

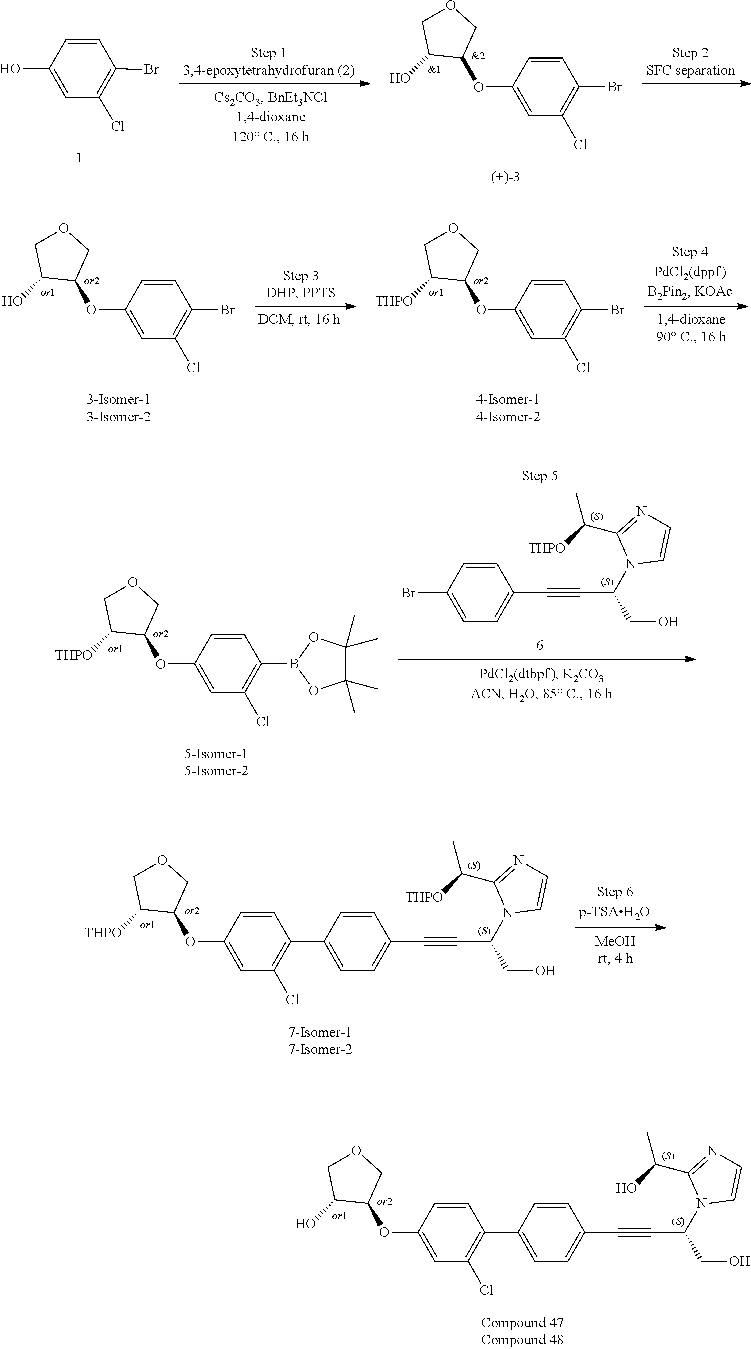

### Step 1:

To a solution of 4-bromo-3-chlorophenol (1, 9.04 g, 43.6 mmol) in 1,4-dioxane (150 mL), were added cesium carbonate (28.4 g, 87 mmol), 3,4-epoxytetrahydrofuran (2, 5 g, 58.1 mmol) and benzyltriethylammonium chloride (2.65 g, 11.62 mmol) at room temperature and the resulting reaction mixture stirred at 120° C. for 16 h. The reaction mixture was cooled to room temperature, filtered through Celite bed; and the bed was washed with EtOAc (2×100 mL). The filtrate was combined and concentrated under reduced pressure. The crude residue, thus obtained, was purified by using MPLC (manually packed SiO₂ cartridge, 230-400 mesh size; 20% EtOAc in hexanes) to afford (±)-3 as colorless gum. Yield: 8.8 g (49%). SFC: enantiomeric ratio=1:1 (tR=2.10 min and 2.67 min; Column: LUX-I-A3; Eluents: CO2 and 0.5% isopropyl amine in MeOH).

### Step 2:

The enantiomers of 8.8 g of (±)-3 were separated by SFC (Column: LUX-I Amylose A3 (250*30) mm, 5 μm; Eluents: CO2 and 0.5% isopropyl amine in MeOH) to afford 3-Isomer-1 and 3-Isomer-2 as off-white solid. Yield: 3-Isomer-1=3.4 g and 3-Isomer-2=3 g. SFC: 3-isomer-1: 100%; tR=2.08 min (Column: LUX-I-A3; Eluents: CO2 and 0.5% isopropyl amine in MeOH). SFC: 3-isomer-2: 100%, tR=4.03 min (Column: CHIRALPAK-AS-H; Eluents: 0.2% formic acid in isopropyl alcohol in acetonitrile). Using the method developed for the mixture of enantiomers (±)-3, the isomer having tR=2.08 min was identified as 3-isomer-1 unambiguously. The other isomer, though analysed using a different method, was identified as 3-isomer-2 based on the process of elimination. Individually, both the isomers were taken for further conversion.

### Step 3:

To a stirred solution of 3-Isomer-1 (3.4 g, 11.58 mmol) in DCM (30 mL), were added 3,4-dihydro-2H-pyran (2.118 mL, 23.17 mmol) and pyridinium p-toluenesulfonate (0.116 g, 0.463 mmol) at room temperature and stirred for 16 h. The reaction mixture was quenched with saturated solution of sodium bicarbonate (15 mL) at 0° C. and extracted with DCM (2×100 mL). The combined organic extract was washed with brine (30 mL), dried over anhydrous Na2SO4, filtered and concentrated under reduced pressure. The crude residue thus obtained was purified by using MPLC (manually packed SiO₂ cartridge, 230-400 mesh size; 12% EtOAc in hexanes) to afford 4-Isomer-1 as a colorless liquid. Yield: 4.0 g (91%).

### Step 4:

To a stirred solution of 4-Isomer-1 (3.8 g, 10.06 mmol) in 1,4-dioxane (40 mL), were added potassium acetate (1.975 g, 20.12 mmol) and bis(pinacolato)diboron (3.83 g, 15.09 mmol) at room temperature. The reaction mixture was purged with nitrogen for 5 min. To this reaction mixture, PdCl2(dppf) (0.736 g, 1.006 mmol) was added and stirred at 90° C. for 16 h. The reaction mixture was cooled to room temperature, quenched with water (20 mL) and extracted with EtOAc (2×40 mL). The combined organic extract was washed with brine (20 mL), dried over anhydrous Na2SO4, filtered and concentrated under reduced pressure. The resulting crude residue was purified by using MPLC (manually packed SiO₂ cartridge, 230-400 mesh size; 10% EtOAc in hexanes) to get 5-Isomer-1 as a gum. Yield: 3.0 g (69%).

### Step 5:

To the stirred solution of 6 (1.0 g, 2.385 mmol) in ACN (10 mL) and water (3.33 mL), were added 5-Isomer-1 (1.013 g, 2.385 mmol) and K₂CO₃ (0.989 g, 7.15 mmol) at room temperature and the reaction mixture purged with nitrogen for 5 min. To this reaction mixture, PdCl₂(dtbpf) (0.311 g, 0.477 mmol) was added and stirred at 85° C. for 16 h. Two more batches were performed with 1 g of 6. All three batches were mixed for work-up and purification. The reaction was cooled to room temperature, quenched with ice-cold water (25 mL) and extracted with 5% MeOH in DCM (3×50 mL). The combined organic extract was washed with brine (25 mL), dried over anhydrous Na2SO4, filtered and concentrated under reduced pressure. The crude residue, thus obtained, was purified by using MPLC (manually packed SiO₂ cartridge, 230-400 mesh size; 4% MeOH in DCM) to get 7-Isomer-1 as a brown sticky solid. Yield: 2.1 g (for 3 batches). LCMS: Calculated for C₃₅H₄₁C₁N₂O₇ is 637.17. Observed: 637.2 [M+]⁺.

### Step 6:

To the stirred solution 7-Isomer-1 (250 mg, 0.392 mmol) in MeOH (15 mL), were added p-toluenesulfonic acid monohydrate (224 mg, 1.177 mmol) at 0° C. and the resulting reaction mixture stirred for 4 h at RT. The reaction mixture was quenched with sat. NaHCO₃ solution (10 mL) at 0° C. The aqueous layer was extracted with DCM (3×100 mL). The combined organic extract was washed with sat. NaHCO₃ solution (2×5 mL) followed by brine solution (10 mL), dried over anhydrous Na2SO4, filtered, and concentrated under reduced pressure. The crude residue thus obtained was purified by using preparative HPLC purification (Column: X-BRIDGE C8 (19*150 mm) 5 μm; Eluents:10 mm ammonium bicarbonate in water and ACN) to obtain Compound 47 as an off-white solid. Yield: 45 mg (24%). LCMS: Calculated for C₂₅H₂₅ClN₂O₅ is 468.93. Observed: 469.0 [M+1]⁺. 1H-NMR (400 MHz, DMSO-d₆): δ 7.54 (d, J=8.4 Hz, 2H), 7.43 (d, J=8.4 Hz, 2H), 7.37-7.35 (m, 2H), 7.23 (d, J=2.4 Hz, 1H), 7.07 (dd, J=2.4, 8.6 Hz, 1H), 6.84 (d, J=0.8 Hz, 1H), 5.68 (t, J=6.0 Hz, 1H), 5.55-5.52 (m, 2H, exchange with D2O), 5.37 (d, J=5.6 Hz, 1H, exchanges with D2O), 4.96-4.93 (m, 1H), 4.75 (d, J=3.6 Hz, 1H), 4.22 (br s, 1H), 4.07 (dd, J=4.4, 10.4 Hz, 1H), 3.94-3.91 (m, 1H), 3.89-3.79 (m, 2H), 3.80 (d, J=10.0 Hz, 1H), 3.60 (dd, J=3.6, 9.2 Hz, 1H), 1.51 (d, J=6.4 Hz, 3H). SFC: 98.5%; tR=6.18 min (Column: LUX-I Amylose-3; Eluents: 0.5% isopropyl amine in acetonitrile and MeOH). trans-geometry at tail. Diastereomeric ratio=98.53:1.46 (based on SFC).

50 mg of Compound 48 was synthesized by using the procedure detailed in Step-3 to Step-6 for Compound 47. LCMS: Calculated for C₂₅H₂₅ClN₂O₅ is 468.15, Observed: 469.0 [M+1]⁺ 1H-NMR (400 MHz, DMSO-d₆): δ 7.54 (d, J=8.4 Hz, 2H), 7.43 (d, J=8.4 Hz, 2H), 7.37-7.35 (m, 2H), 7.23 (d, J=2.4 Hz, 1H), 7.07 (dd, J=2.8, 8.6 Hz, 1H), 6.84 (d, J=1.2 Hz, 1H), 5.69 (t, J=6.0 Hz, 1H), 5.55-5.52 (m, 2H, exchange with D2O), 5.37 (d, J=5.6 Hz, 1H, exchanges with D2O), 4.98-4.91 (m, 1H), 4.75 (d, J=3.6 Hz, 1H), 4.23 (br s, 1H), 4.09-4.05 (m, 1H), 3.94-3.91 (m, 1H), 3.87 (t, J=6.0 Hz, 2H), 3.80 (d, J=10.0 Hz, 1H), 3.60 (dd, J=1.6, 9.4 Hz, 1H), 1.51 (d, J=6.4 Hz, 3H). SFC: 99.59%; tR=8.92 min (Column: LUX-I Amylose-3; Eluents: CO2 and 0.5% isopropyl amine in acetonitrile and MeOH). trans-geometry at tail. Diastereomeric ratio=99.59:0.4.

### Example A10: Synthesis of Compounds 49 and 50

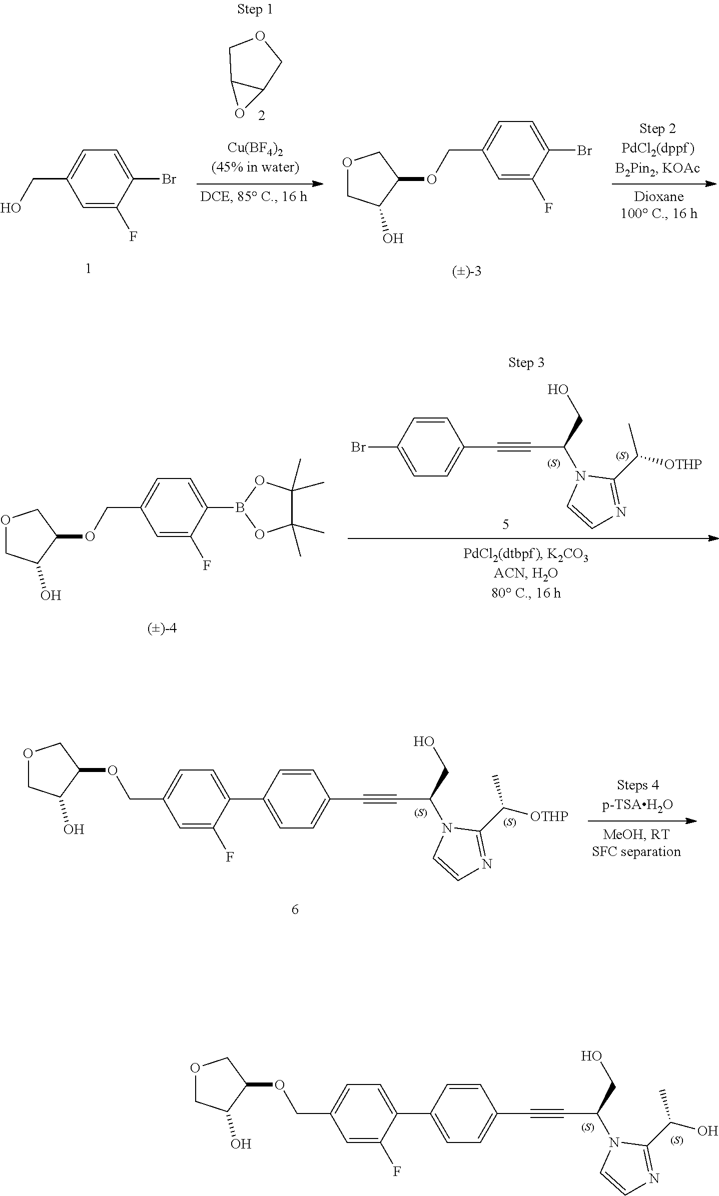

### Step 1:

To a stirred solution of 3,4-epoxytetrahydrofuran (2, 10.50 g, 122 mmol) in DCE (300 mL), was added (4-bromo-3-fluorophenyl)methanol (1, 10 g, 48.8 mmol) at room temperature. To this reaction mixture, was added copper(II) tetrafluoroborate (3.34 mL, 9.75 mmol) at room temperature and the resulting reaction mixture stirred at 85° C. for 16 h. The reaction mixture was quenched with water (250 mL) and extracted with DCM (100 mL×2). The combined organic layer was washed with brine (50 mL), dried over anhydrous Na₂SO₄, filtered and concentrated under reduced pressure. The crude residue thus obtained, was purified by using MPLC (manually packed cartridge, SiO₂ 230-400 mesh; 40% EtOAc in hexanes) to (±)-3 as a pale-yellow solid. Yield: 1.3 g (8%).

### Step 2:

To a stirred solution of (±)-3 (3 g, 10.31 mmol) in 1,4-Dioxane (80 mL), were added bis(pinacolato)diboron (3.93 g, 15.46 mmol) and potassium acetate (3.03 g, 30.9 mmol). The reaction mixture was degassed for 5 min following which PdCl2(dppf) (0.754 g, 1.031 mmol) was added. The mixture was degassed again with nitrogen for 2 min and then stirred at 100° C. for 16 h. The reaction mixture was quenched with water (100 mL) and extracted with EtOAc (100 mL×2). The combined organic layers were washed with brine (50 mL), dried over anhydrous Na₂SO₄, filtered and concentrated under reduced pressure. The crude residue was purified by using MPLC (manually packed cartridge, SiO₂ 230-400 mesh; 30% EtOAc in Hexanes) to afford (±)-4 as a pale-yellow solid. Yield: 2 g (53%).

### Step 3:

To a stirred solution of 5 (3.1 g, 7.39 mmol) in acetonitrile (60 mL) and water (60 mL), were added (±)-4 (3.63 g, 10.72 mmol) and K2CO3 (3.07 g, 22.18 mmol) at room temperature. The reaction mixture was purged for 5 min with nitrogen. To this reaction mixture, was added PdCl2(dtbpf) (0.482 g, 0.739 mmol) and the purging continued for 2 min and then stirred at 80° C. for 16 h. The reaction mixture was quenched with water (50 mL) and extracted with 10% MeOH in DCM (50 mL×3). The combined organic layer was washed with brine (20 mL), dried over anhydrous Na₂SO₄, filtered and concentrated under reduced pressure. The resulting crude residue was purified by MPLC (manually packed cartridge, SiO₂ 230-400 mesh; 6% MeOH in DCM) to afford 6 as a brown solid. Yield: 2 g (40%). LC-MS: Calculated for C31H35FN2O6 is 550.6, Observed: 551.2 [M+1]⁺.

### Step 4:

To a stirred solution of 6 (2 g, 3.63 mmol) in MeOH (90 mL), was added p-toluenesulfonic acid monohydrate (2.073 g, 10.90 mmol) at room temperature. The resulting reaction mixture was stirred at room temperature for 2 h. The volatiles in the reaction were removed under reduced pressure and the crude residue basified with sat. NaHCO3 solution (50 mL). The aqueous layer was extracted with 20% MeOH in DCM (50 mL×3). The combined organic extract was dried over anhydrous Na2SO4, filtered and concentrated to afford a crude mass which was purified by preparative HPLC (Column: X-BRIDGE C8 (19×150 mm) 5 μm; Eluents:10 mM ammonium bicarbonate in water and ACN) to obtain the product as a pale-yellow solid. Yield: 0.5 g (53%). LC-MS: Calculated for C26H27FN2O5 is 466.5, Observed: 467.3 [M+1]⁺. The product was a mixture of diastereomers; racemic at tail.

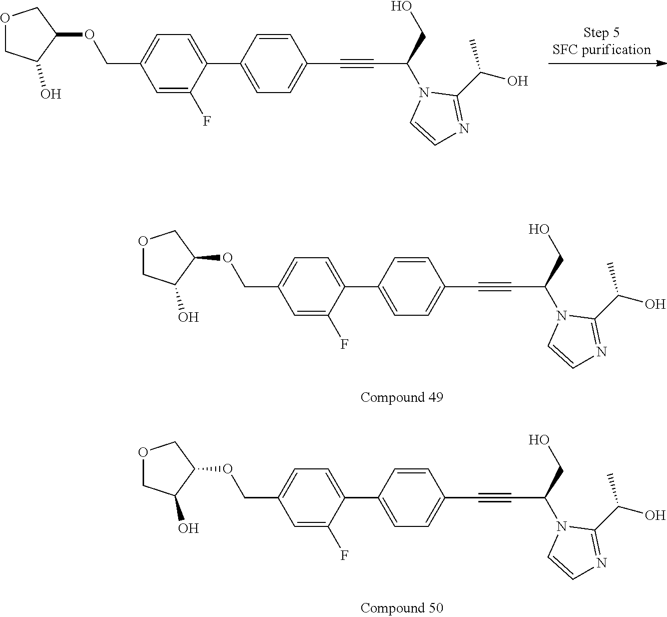

### Step 5:

500 mg of the product was separated SFC purification by using (Column: Chiralpak IH (250×20) mm, 5 μm; Eluents: CO₂:0.5% isopropylamine in IPA [65:35]) to get Compound 49* (t_{R}=2.40 min) as an off-white solid and Compound 50* (t_{R}=5.62 min) as an off -white solid. (* denotes arbitrarily assigned stereochemistry).

Compound 49: Yield: 159 mg. LC-MS: Calculated for C26H27FN2O5 is 466.5, Observed: 467.3 [M+1]⁺. SFC purity=100%. Diastereomeric excess (de)=100%

Compound 50: Yield: 153 mg. LC-MS: Calculated for C₂₆H27FN2O5 is 466.5, Observed: 467.3 [M+1]⁺. SFC purity=99.7%. Diastereomeric excess (de)=99.5%

### Example A11: Synthesis of Compounds 51, 52, and 53

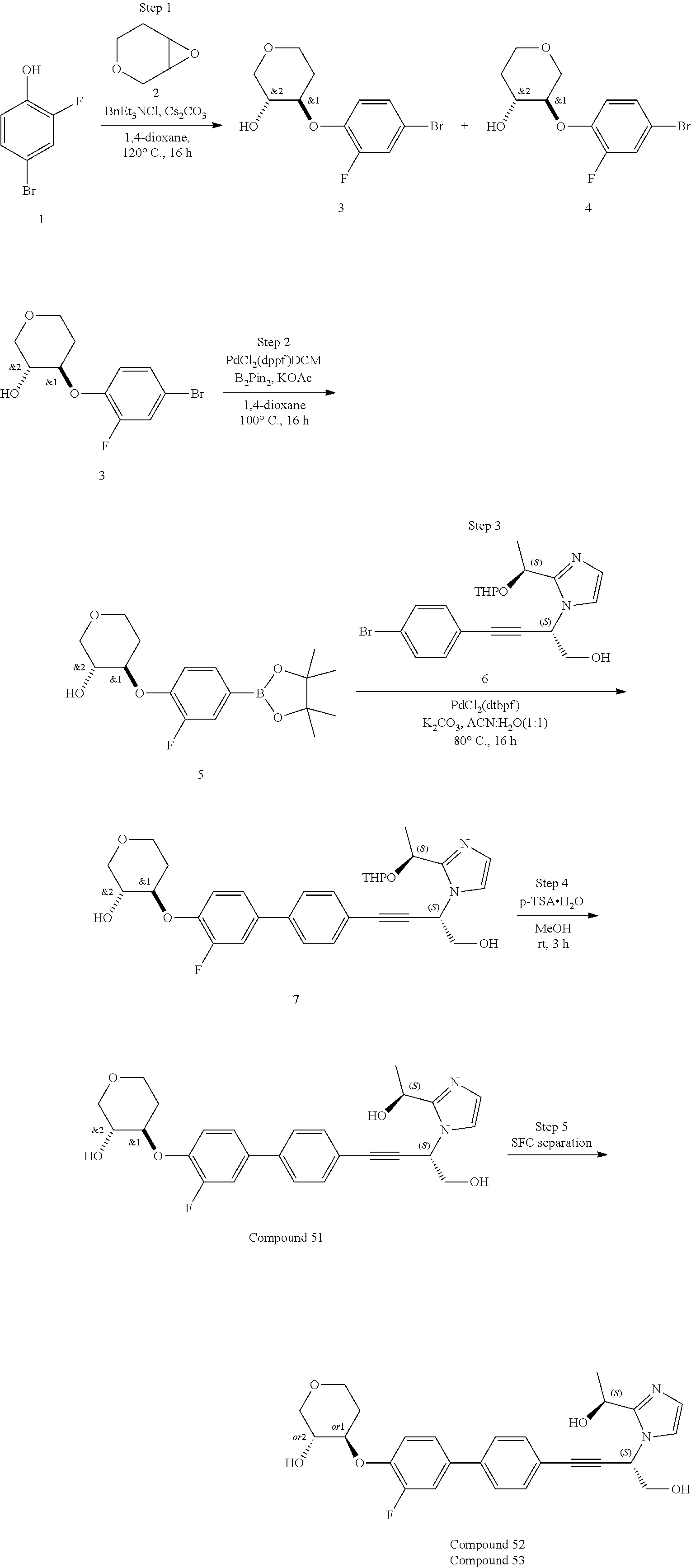

### Step 1:

To a stirred solution of 4-bromo-2-fluorophenol (1, 7.63 g, 40.0 mmol) in 1,4-dioxane (120 mL), were added caesium carbonate (24.41 g, 74.9 mmol), 2 (5 g, 49.9 mmol), and benzyltriethylammonium chloride (2.275 g, 9.99 mmol) at room temperature. After complete addition, the resulting reaction mixture was heated to 120° C. and stirred for 16 h. One more batch with 7.63 g of 1 was performed. Both batches were mixed for work-up and purification. The reaction mixture was cooled to room temperature, filtered through a Celite bed. The Celite bed was washed with EtOAc (2×100 mL). The combined filtrate was concentrated under reduced pressure. The obtained crude residue was purified by MPLC (using manually packed SiO₂ cartridge 230-400 mesh size; 20% EtOAc in hexanes) to afford 7 g of mixture of 3 and 4 as colorless gum. Further, the mixture of 3 and 4 (3.1 g, 5.32 mmol) was re-purified by MPLC (using manually packed SiO₂ cartridge, 230-400 mesh; 3% EtOAc in DCM) to afford 3 as a colorless liquid (polar spot on TLC) and 4 as an off-white solid (non-polar spot on TLC). The identity of the required isomer was done through 2D experiments. Yield: 3=2.6 g and 4=450 mg.

### Step 2:

To a stirred solution of 3 (2.6 g, 8.93 mmol) in 1,4-dioxane (40 mL), was added potassium acetate (2.191 g, 22.33 mmol) and bis(pinacolato)diboron (5.67 g, 22.33 mmol) at room temperature. The resulting reaction mixture was purged with nitrogen for 5 min. To this reaction mixture, PdCl2(dppf)DCM (0.729 g, 0.893 mmol) was added the resulting reaction mixture was heated to 100° C. and for 16 h. The reaction mixture was cooled to room temperature, diluted with EtOAc (15 mL), and filtered through a Celite bed. The Celite bed was washed with EtOAc (2×15 mL). The combined filtrate was washed with water (15 mL). The aqueous layer was extracted with EtOAc (3×80 mL). The combined extract was washed with brine (60 mL), dried over anhydrous sodium sulfate, filtered, and concentrated under reduced pressure. The crude residue was purified by MPLC (using manually packed SiO2 cartridge, 230-400 mesh size; 30% EtOAc in hexanes) to afford 5 as an off-white solid. Yield: 2.7 g (71%).

### Step 3:

To a stirred solution of 6 (1.5 g, 4.44 mmol) in acetonitrile (25 mL) and water (25 mL), were added 5 (2.79 g, 6.65 mmol) and potassium carbonate (1.839 g, 13.31 mmol) at room temperature. The reaction mixture was purged with nitrogen for 5 min. To this reaction mixture, PdCl2(dtbpf) (0.289 g, 0.444 mmol) was added at room temperature. The resulting reaction mixture was heated to 80° C. and stirred for 16 h. The reaction mixture was cooled to room temperature, quenched with water (50 mL) and extracted with EtOAc (3×90 mL). The combined organic extract was washed with brine (50 mL), dried over sodium sulfate, filtered and the filtrate was concentrated under reduced pressure. The obtained crude residue was purified by MPLC (using manually packed SiO2 cartridge, 230-400 mesh size; 5% MeOH in DCM) to afford 7 as a brown solid. Yield: 950 mg (38%). LCMS: Calculated for C31H35FN2O6 is 550.63, Observed: 551.4 [M+1]⁺.

### Step 4:

To a stirred solution of 7 (950 mg, 1.725 mmol) in MeOH (20 mL), was added p-toluenesulfonic acid monohydrate (985 mg, 5.18 mmol) at 0° C. The resulting reaction mixture was stirred at room temperature and for 3 h. The reaction was quenched with sat. NaHCO3 solution (20 mL) at 0° C. and extracted with 10% MeOH in DCM (3×50 mL). The combined organic extract was washed with brine (30 mL), dried over anhydrous sodium sulphate, filtered, and concentrated. The obtained crude residue was purified by MPLC (using manually packed SiO2 cartridge, 230-400 mesh size; 6% MeOH in DCM) to afford Compound 51 as a brown solid. Yield: 460 mg (57%). LCMS: Calculated for C26H27FN2O5 is 466.51, Observed: 467.2 [M+1]⁺.

### Step 5:

The diastereomers of 450 mg of Compound 51 were separated by SFC (Column: I-AMYLOSE-A (250*20) mm, 5N μm; Eluents: CO2 and 0.5% isopropylamine in MeOH (70:30)) to afford Compound 52 and Compound 53 as off-white solids.

Yield: Compound 52=200 mg and Compound 53=180 mg.

Compound 52: LCMS: Calculated for C26H27FN2O5 is 466.51, Observed: 467.2 [M+1]⁺. 1H-NMR (400 MHz, DMSO-d6): δ 7.70 (dd, J=1.60, 6.80 Hz, 2H), 7.61 (dd, J=2.00, 12.80 Hz, 1H), 7.52 (dd, J=1.60, 6.80 Hz, 2H), 7.48 (dd, J=1.60, 8.80 Hz, 1H), 7.42-7.37 (m, 1H), 7.36 (app d, J=1.20 Hz, 1H), 6.84 (app d, J=1.20 Hz, 1H), 5.69 (t, J=6.00 Hz, 1H), 5.54 (t, J=6.00 Hz, 1H, exchanges with D2O), 5.37 (d, J=5.60 Hz, 1H, exchanges with D2O), 5.32 (d, J=5.20 Hz, 1H, exchanges with D2O), 4.96-4.92 (m, 1H), 4.37-4.35 (m, 1H), 3.87 (t, J=6.00 Hz, 2H), 3.84-3.79 (m, 2H), 3.60-3.59 (m, 1H), 3.46-3.41 (m, 1H), 3.17 (dd, J=8.80, 11.40 Hz, 1H), 2.10-2.07 (m, 1H), 1.57-1.50 (m, 4H).

l-Amylose-A_0.5% IPAm in MeOH tR=7.51 min. Unknown stereochemistry at tail part, single isomer.

Compound 53: 1H-NMR (400 MHz, DMSO-d6): δ 7.70 (d, J=8.40 Hz, 2H), 7.61 (dd, J=2.40, 12.80 Hz, 1H), 7.52 (d, J=8.40 Hz, 2H), 7.48 (dd, J=1.60, 8.80 Hz, 1H), 7.42-7.37 (m, 1H), 7.36 (app d, J=1.20 Hz, 1H), 6.84 (app d, J=1.20 Hz, 1H), 5.69 (t, J=6.00 Hz, 1H), 5.54 (t, J=6.00 Hz, 1H, exchanges with D2O), 5.37 (d, J=5.60 Hz, 1H, exchanges with D2O), 5.32 (d, J=5.60 Hz, 1H, exchanges with D2O), 4.96-4.92 (m, 1H), 4.36-4.35 (m, 1H), 3.87 (t, J=6.00 Hz, 2H), 3.84-3.79 (m, 2H), 3.60-3.59 (m, 1H), 3.44 (dd, J=8.80, 10.80 Hz, 1H), 3.17 (dd, J=8.80, 11.20 Hz, 1H), 2.10-2.07 (m, 1H), 1.57-1.50 (m, 4H).

l-Amylose-A_0.5% IPAm in MeOH tR=11.35 min. Unknown stereochemistry at tail part diastereomeric ratio=98.7:1.3.

### Example A12: Synthesis of Compounds 54, 55, and 56

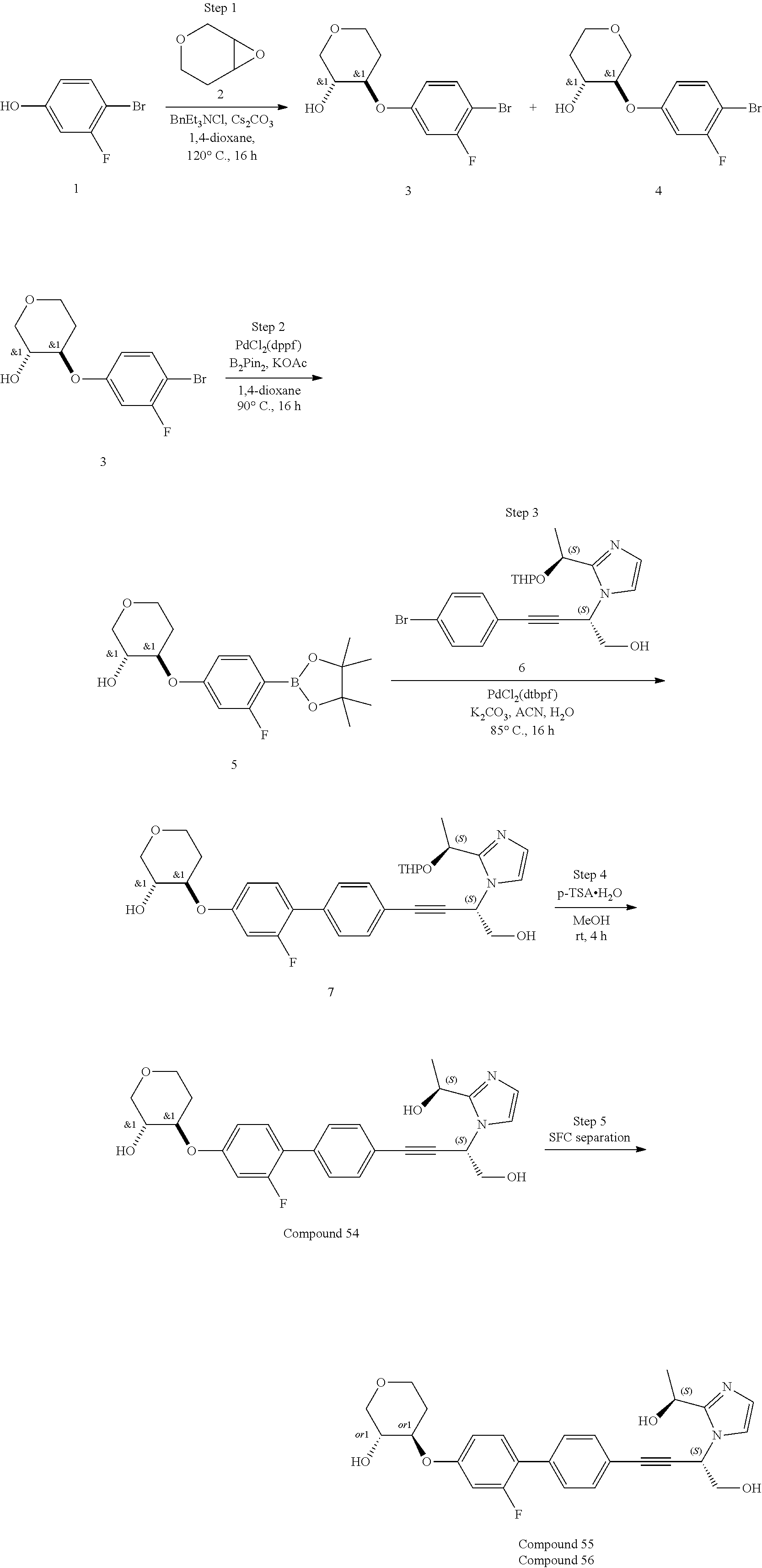

### Step 1:

To a stirred solution of 4-bromo-3-fluorophenol (1, 5 g, 26.2 mmol) in 1,4-dioxane (120 mL), were added cesium carbonate (17.06 g, 52.4 mmol), 3,7-dioxabicyclo[4.1.0]heptane (2, 3.41 g, 34.0 mmol) and benzyltriethylammonium chloride (1.193 g, 5.24 mmol) at room temperature. The resulting reaction mixture was stirred at 120° C. for 16 h. One more batch was performed with 5 g of 1. Both batches were mixed for work-up and purification. The reaction was cooled to room temperature, filtered through Celite bed, and the Celite bed was washed with EtOAc (2×100 mL). The combined filtrate was concentrated under reduced pressure. The crude residue was purified by MPLC (using manually packed SiO₂ cartridge, 230-400 mesh size; 20% EtOAc in hexanes) afforded 10 g of mixture of 3 and 4.

Further, isomers (3 and 4) were separated by using MPLC (manually packed SiO₂ cartridge 230-400 mesh size; 3% EtOAc in DCM) afforded 4.5 g of 3 as a colorless gum. Yield: 4.5 g (from both batches). The structure of 3 was confirmed from 2D NMR (COSY and HSQC) experiments.

### Step 2:

To a stirred solution of 3 (1.1 g, 3.78 mmol) in 1,4-dioxane (10 mL), were added potassium acetate (1.113 g, 11.34 mmol) and bis(pinacolato)diboron (1.919 g, 7.56 mmol) at room temperature. The reaction mixture was purged with nitrogen for 5 min. To this reaction mixture, PdCl2(dppf) (0.276 g, 0.378 mmol) was added and the resulting reaction mixture heated to 90° C. for 16 h. The reaction mixture was then cooled to room temperature, diluted with EtOAc (10 mL) and filtered through a Celite pad. The Celite pad was washed with EtOAc (20 mL×2). The combined filtrate was washed with brine solution (20 mL), dried over anhydrous sodium sulphate, filtered, and concentrated under reduced pressure. The crude residue was purified by MPLC (using manually packed SiO2 cartridge, 230-400 mesh size; 25% EtOAc in hexanes) to afford 5 as an off-white solid. Yield: 1.3 g (81%).

### Step 3:

To the stirred solution of 6 (1.0 g, 2.385 mmol) in acetonitrile (3 mL) and water (3 mL), were added K2CO3 (0.989 g, 7.15 mmol) and 5 (1.048 g, 3.10 mmol) at room temperature. The reaction mixture was purged with N₂ for 5 min. To this reaction mixture, PdCl2(dtbpf) (0.311 g, 0.477 mmol) was added and the resulting reaction mixture was heated to 85° C. for 16 h. The reaction mixture was quenched with ice water (10 mL) and extracted with 5% MeOH in DCM (30 mL×3). The combined organic extract was washed with brine solution (10 mL), dried over anhydrous sodium sulphate, filtered, and concentrated under reduced pressure. The obtained crude residue was purified by MPLC (using manually packed SiO2 cartridge; 230-400 mesh size; 5% MeOH in DCM) to get 7 as brown sticky solid. Yield: 0.9 g (55%). LCMS: Calculated for C₃₁H₃₅FN₂O₆ is 550.63, Observed: 551.3 [M+1]⁺.

### Step 4:

To the stirred solution of 7 (0.3 g, 0.545 mmol) in MeOH (30 mL), was added p-toluenesulfonic acid monohydrate (0.311 g, 1.634 mmol) at room temperature. The resulting reaction mixture was stirred for 4 h. The reaction mixture was quenched with sat. NaHCO3 solution at 0° C. Three more batches were performed, each using on 300 mg of 7. All the batches were mixed for work-up and purification. The combined suspension was extracted with DCM (100 mL×3). The combined organic extract was washed with sat. NaHCO₃ solution (3×15 mL) and brine solution (15 mL), dried over anhydrous sodium sulphate, filtered, and concentrated under reduced pressure. The obtained crude residue was purified by reverse phase preparative HPLC (Column: Shimpack C18 (150*20) 5 μm, Eluents: 10 mM ammonium bicarbonate in water and acetonitrile) to afford Compound 54 as an off-white solid. Yield: 250 mg (combined yield for four batches). By using above procedures another 100 mg of Compound 54 was synthesized. Both batches were blended to obtain 350 mg of Compound 54. Calculated for C₂₆H₂₇FN₂O₅ is 466.51, Observed: 467.3 [M+1]⁺. 1H-NMR (400 MHz, DMSO-d6): δ 7.54 (m, 4H), 7.46 (t, J=9.20 Hz, 1H), 7.36 (app d, J=0.80 Hz, 1H), 7.05 (dd, J=2.40, 13.20 Hz, 1H), 6.96 (dd, J=2.40, 8.60 Hz, 1H), 6.84 (app d, J=1.20 Hz, 1H), 5.70 (t, J=6.00 Hz, 1H), 5.53 (t, J=5.60 Hz, 1H, exchanges with D2O), 5.37 (d, J=5.60 Hz, 1H, exchanges with D2O), 5.28 (d, J=5.20 Hz, 1H, exchanges with D2O), 4.94 (t, J=6.40 Hz, 1H), 4.36 (m, 1H), 3.85-3.77 (m, 4H), 3.58-3.54 (m, 1H), 3.45 (t, J=2.40 Hz, 1H), 3.17 (dd, J=8.80, 11.20 Hz, 1H), 2.11 (d, J=10.40 Hz, 1H), 1.54-1.50 (m, 4H). 19F (MHz, DMSO-d6): δ −115.764. LUX Amylose-1_0.5% IPAm in MeOH (tR=3.65 and 5.86 min).

### Step 5:

The diastereomers of Compound 54 (335 mg) were separated by SFC (Column: Lux A3-(250*20) mm, 5 μm; Eluents: CO2 and 0.5% isopropylmine in ACN:MeOH (60:40)) to afford Compound 55 and Compound 56 as off-white solids. Yield: Compound 55=98 mg and Compound 56=220 mg.

90 mg of impure Compound 55 was repurified by using MPLC (manually packed SiO2 cartridge: 230-400 mesh size; 6% MeOH in DCM) to afford 43 mg of Compound 55 as an off-white solid. The 1H NMR data of Compound 55 was identical to Compound 54. Unknown stereochemistry at tail part; diastereomeric ratio=96.7:0.3; LUX Amylose-1_0.5% IPAm in MeOH; tR=3.43 min.

190 mg of impure Compound 56 was re-purified by reverse phase preparative HPLC (Column: X-SELECT C18 (250*19 mm) 5 μm; Eluents: 0.1% ammonium bicarbonate in water and acetonitrile) to afford 26 mg of Compound 56 as an off-white solid. The 1H NMR data of Compound 56 was identical to Compound 54. Unknown stereochemistry at tail part; diastereomeric ratio=99.7:0.3; LUX Amylose-1_0.5% IPAm in MeOH; tR=5.67 min.

### Example A13: Synthesis of Compounds 57 and 58

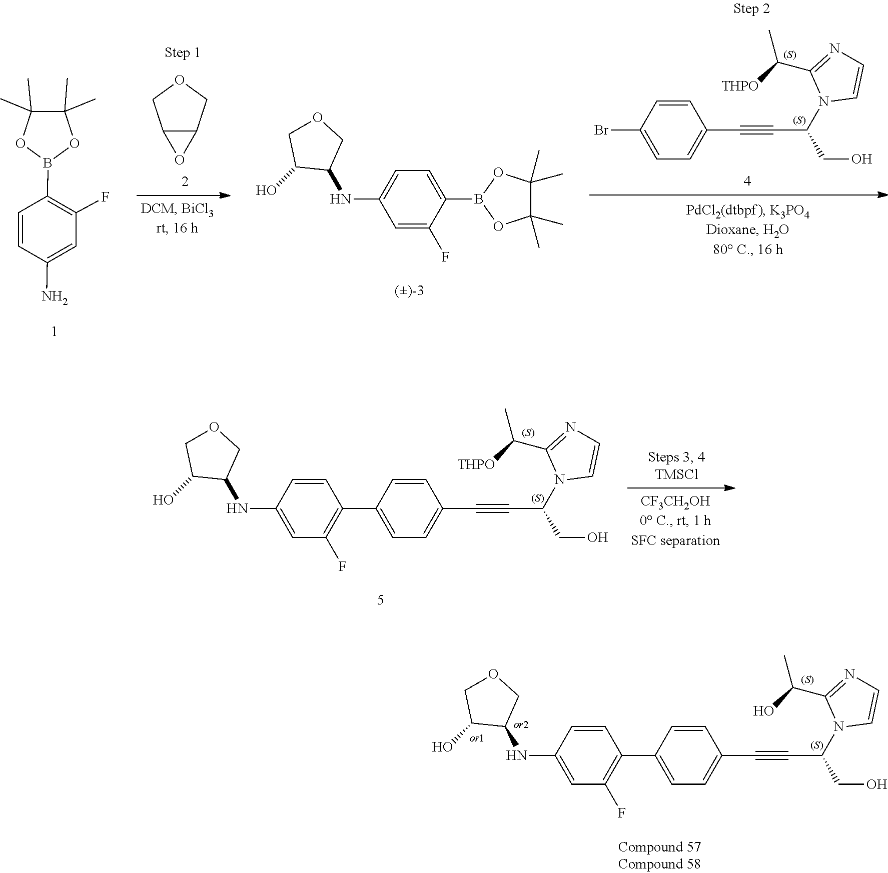

### Step-1:

To a stirred solution of 3-fluoro-4-(4,4,5,5-tetramethyl-1,3,2-dioxaborolan-2-yl)aniline (1, 5.0 g, 21.09 mmol) in DCM (50 mL), were added 3,4-epoxytetrahydrofuran (2, 1.476 mL, 21.09 mmol) and bismuth (III) chloride (0.665 g, 2.109 mmol) at room temperature. The reaction mixture was stirred at room temperature for 16 h. The reaction mixture was quenched with saturated NaHCO₃ solution (150 mL). The inorganic solids were filtered through Celite pad. The Celite bed was then washed with DCM (4×150 mL). The combined organic layer was washed with brine (150 mL), dried over anhydrous sodium sulphate, filtered and concentrated under reduced pressure. The resulting crude product was purified by MPLC (using manually packed SiO2 cartridge, 230-400 mesh size; 25% EtOAc in hexanes) to afford (±)-3 as an off white solid. Yield: 4.0 g (51%). LC-MS: Calculated for C₁₆H₂₃BFNO₄ is 323.17, Observed: 324.2 [M+1]⁺.

### Step-2:

To a stirred solution of 4 (3.24 g, 7.74 mmol) in dioxane (20 mL) and water (5 mL), were added (±)3 (2.5 g, 7.74 mmol) and potassium phosphate tribasic (3.28 g, 15.47 mmol) at room temperature. The reaction mixture was purged with nitrogen for 5 min. To this reaction mixture, PdCl2(dtbpf) (0.504 g, 0.774 mmol) was added and the purging continued for another 2 min. The resulting reaction mixture was stirred at 80° C. for 16 h. The inorganic solids were filtered off through a Celite pad and washed with 10% MeOH in DCM (3×150 mL). The filtrate was concentrated under reduced pressure. The resulting residue was purified by MPLC (using manually packed cartridge SiO₂ 230-400 mesh size; 5% MeOH in EtOAc) to afford 5 as a brown solid. Yield: 1.79 g (40%). LC-MS: Calculated for C₃₀H₃₄FN₃O₅ is 535.62, Observed: 536.2 [M+1]⁺.

### Step-3:

To a stirred solution of 5 (1.64 g, 3.06 mmol) in trifluoroethanol (17 mL), was added TMSCl (0.270 mL, 3.06 mmol) at 0° C. and the reaction mixture stirred at room temperature for 1 h. The volatiles were then evaporated under reduced pressure. To the resulting residue, saturated NaHCO₃ solution (50 mL) was added and extracted with EtOAc (3×100 mL). The combined organic layer was washed with brine (50 mL), dried over anhydrous sodium sulphate, filtered and concentrated under reduced pressure. The resulting crude product was purified by MPLC (using manually packed SiO₂ cartridge 230-400 mesh size; 5% MeOH in DCM) to afford 5 as a brown solid. Yield: 0.57 g (41%). LC-MS: Calculated for C₂₅H₂₆FN₃O₄ is 451.49, Observed: 452.3 [M+1]⁺.

### Step-4:

The diastereomers of 570 mg of 5 were separated by SFC (Column: Lux A3 (250*20) mm, 5 μm; Eluents: CO₂:0.5% isopropyl amine in methanol [60:40]) to get Compound 57 and Compound 58 as white solids. Yield: Compound 57 (tR=8.37 min)=176 mg and Compound 58 (tR=9.97 min)=130 mg. Impure Compound 58 was repurified by reversed phase chromatography (Column: Redisep Gold; C18 SiO₂; Eluents: 28% 10 mM ammonium bicarbonate in water: ACN) to afford Compound 58. Yield: Compound 58 (tR=9.97 min)=120 mg.

Compound 57: LC-MS: Calculated for C₂₅H₂₆FN₃O₄ is 451.49, Observed: 452.3 [M+1]⁺. 1H-NMR (400 MHz, DMSO-d6): δ 7.49 (s, 4H), 7.35 (d, J=1.20 Hz, 1H), 7.29 (t, J=9.20 Hz, 1H), 6.84 (d, J 1.20 Hz, 1H), 6.59 (dd, J=2.00, 8.80 Hz, 1H), 6.54 (dd, J=2.00, 14.40 Hz, 1H), 6.40 (d, J=5.60 Hz, 1H, exchanges with D2O), 5.69 (t, J=6.00 Hz, 1H), 5.53 (t, J=5.60 Hz, 1H, exchanges with D2O), 5.37 (d, J=5.60 Hz, 1H, exchanges with D2O), 5.34 (d, J=3.60 Hz, 1H, exchanges with D2O), 4.95-4.92 (m, 1H), 4.06-4.04 (m, 2H), 3.88-3.84 (m, 3H), 3.67-3.62 (m, 2H), 3.57 (dd, J=1.20, 9.20 Hz, 1H), 1.51 (d, J=6.40 Hz, 3H). 19F-NMR (376. MHz, DMSO-d6): δ −117.2 (dd, J=9.79, 13.94 Hz). Unknown stereochemistry in tail part; single isomer with SFC purity=100% (LUX-l-Amylose3_0.5% IPAm in MeOH tR=8.37 min).

Compound 58: LC-MS: Calculated for C₂₅H₂₆FN₃O₄ is 451.49, Observed: 452.3 [M+1]⁺. 1H-NMR (400 MHz, DMSO-d6): δ 7.49 (s, 4H), 7.35 (d, J=1.20 Hz, 1H), 7.29 (t, J=9.20 Hz, 1H), 6.84 (d, J=1.20 Hz, 1H), 6.59-6.52 (m, 2H), 6.40 (d, J=5.60 Hz, 1H, exchanges with D2O), 5.69 (t, J=6.00 Hz, 1H), 5.53 (t, J=5.60 Hz, 1H, exchanges with D2O), 5.37 (d, J=5.60 Hz, 1H, exchanges with D2O), 5.34 (d, J=3.60 Hz, 1H, exchanges with D2O), 4.97-4.91 (m, 1H), 4.06-4.04 (m, 2H), 3.88-3.84 (m, 3H), 3.67-3.62 (m, 2H), 3.57 (dd, J=1.60, 9.60 Hz, 1H), 1.51 (d, J=6.40 Hz, 3H). 19F-NMR (376. MHz, DMSO-d6): δ −117.2 (dd, J=9.79, 13.94 Hz). Unknown stereochemistry in tail part; dr=99:1 (LUX-l-Amylose3_0.5% IPAm in MeOH tR=9.97 min).

### Example A14: Synthesis of Compounds 59 and 60

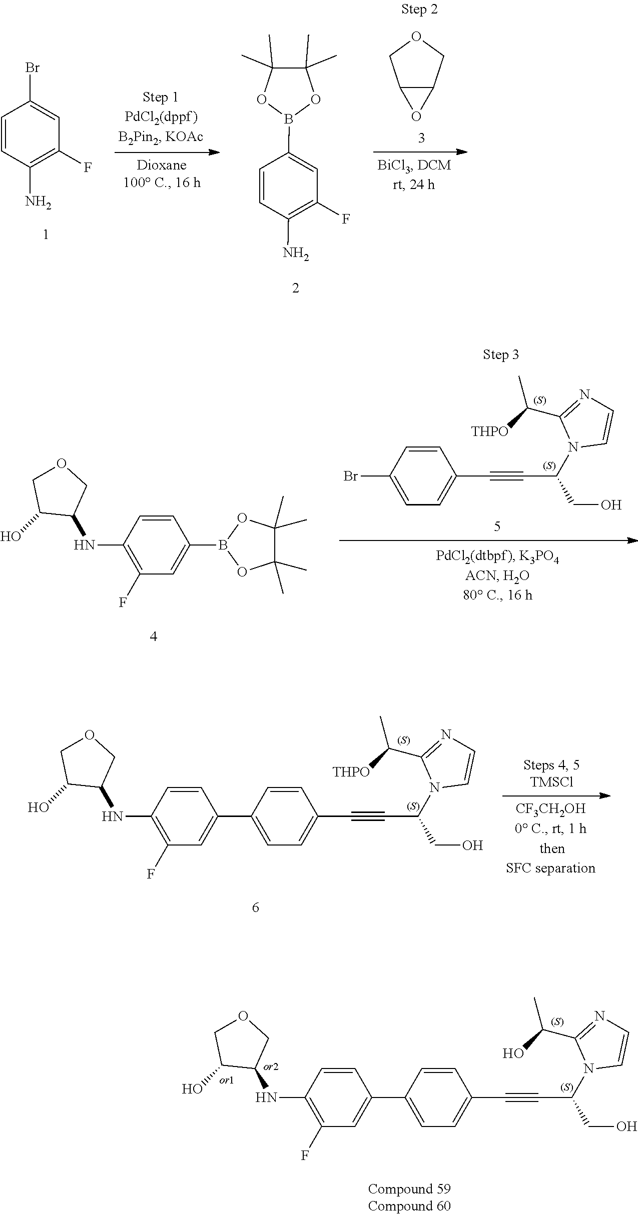

### Step-1:

To a stirred solution of 4-bromo-2-fluoroaniline (1, 10 g, 52.6 mmol) in dioxane (100 mL), were added potassium acetate (10.33 g, 105 mmol) and bis(pinacolato)diboron (14.70 g, 57.9 mmol) at room temperature. The resulting mixture was purged with nitrogen for 5 min. To this reaction mixture, PdCl₂(dppf) (3.85 g, 5.26 mmol) was added and the purging continued for another 2 min. The resulting reaction mixture was stirred at 100° C. for 16 h. The inorganic solids were filtered through a Celite pad and washed with EtOAc (1 L). The filtrate was washed with brine (1 L), dried over anhydrous Na₂SO₄, filtered and concentrated reduced pressure. The resulting crude mass was purified by MPLC (using manually packed SiO₂ cartridge, 230-400 mesh size; 10% EtOAc in hexanes) to afford 2 as an orange solid. Yield: 12.2 g (92%). LC-MS: Calculated for C₁₂H₁₇BFNO₂ is 237.08, Observed: 238.2 [M+1]⁺.

### Step-2:

Procedure adapted from Tet. Lett., 43 (2002) 7891-7893. To a stirred solution of 2 (6.9 g, 29.1 mmol) in DCM (60 mL), were added 3,4-epoxytetrahydrofuran (3, 2.037 mL, 29.1 mmol) and bismuth (III) chloride (0.918 g, 2.91 mmol) at room temperature and the reaction mixture was stirred for 24 h at the same temperature. The reaction mixture was quenched with saturated NaHCO₃ solution (150 mL). The inorganic solids were filtered through Celite pad, washed with EtOAc (2×500 mL). The combined filtrate was washed with brine (200 mL), dried over anhydrous sodium sulphate, filtered and concentrated under reduced pressure. The resulting crude product was purified by MPLC (using manually packed SiO2 cartridge, 230-400 mesh size; 25% EtOAc in hexanes) to afford 4 as a brown liquid. Yield: 4.95 g (39%). LC-MS: Calculated for C₁₆H₂₃BFNO₄ is 323.17, Observed: 324.0 [M+1]⁺.

### Step-3:

To a stirred solution of 5 (2.60 g, 6.19 mmol) in dioxane (15 mL) and water (5 mL), were added 4 (2.0 g, 6.19 mmol) and potassium phosphate tribasic (2.63 g, 12.38 mmol) at room temperature. The reaction mixture was purged with nitrogen for 5 min. To this reaction mixture, PdCl2(dtbpf) (0.403 g, 0.619 mmol) was added and the purging continued for another 2 min. The resulting reaction mixture was stirred at 80° C. for 16 h. The inorganic solids were filtered through a Celite pad and washed with EtOAc (3×250 mL). The filtrate was concentrated under reduced pressure. The crude mass thus obtained was purified by MPLC (using manually packed cartridge SiO₂ 230-400 mesh size; 7% MeOH in EtOAc) to afford 6 as an orange solid. Yield: 1.75 g (51%). LC-MS: Calculated for C₃₀H₃₄FN₃O₅ is 535.62, Observed: 536.0 [M+1]⁺.

### Step-4:

To a stirred solution of 6 (1.79 g, 3.34 mmol) in 2,2,2-trifluoroethanol (17 mL), was added trimethylsilyl chloride (0.424 mL, 3.34 mmol) at 0° C. The reaction mixture was stirred at room temperature for 1 h. The volatiles in the reaction mixture were removed under reduced pressure. The residue obtained was basified with saturated NaHCO₃ solution (50 mL), extracted with EtOAc (2×150 mL). The combined organic layer was washed with brine (50 mL), dried over anhydrous sodium sulphate, filtered and concentrated under reduced pressure. The resulting crude product was purified by reversed phase preparative HPLC (Column: X bridge 5 mm C8 (250*19) μm; Eluents: 10 mM ammonium bicarbonate in water and ACN) to afford 6 (racemic at tail) as an off-white solid. Yield: 0.9 g (59%). LC-MS: Calculated for C₂₅H₂₆FN₃O₄ is 451.49, Observed: 452.3 [M+1]⁺.

### Step-5:

The diastereomers of 6 (racemic at tail) (0.7 g) were separated by SFC (PIC 22-017, Lux A1 (250*20) mm, 5 μm; Eluents: CO2:0.5% isopropyl amine in IPA [65:35]). The fractions were concentrated under reduced pressure to afford Compound 59 (tR=4.63 min) and Compound 60 (tR=6.06 min). Yield: Compound 59 (tR=4.63 min)=130 mg and Compound 60 (tR=6.06 min)=180 mg

Compound 59: LC-MS: Calculated for C₂₅H₂₆FN₃O₄ is 451.49, Observed: 452.3 [M+1]⁺. 1H-NMR (400 MHz, DMSO-d6): δ 7.65 (d, J=8.40 Hz, 2H), 7.49-7.40 (m, 4H), 7.35 (app d, J=1.20 Hz, 1H), 6.93 (t, J=8.80 Hz, 1H), 6.84 (app d, J=0.80 Hz, 1H), 5.67 (m, 2H, 1H exchanges with D2O), 5.53 (t, J=5.60 Hz, 1H, exchanges with D2O), 5.37 (d, J=5.60 Hz, 1H, exchanges with D2O), 5.32 (d, J=4.00 Hz, 1H, exchanges with D2O), 4.95-4.92 (m, 1H), 4.13 (br s, 1H), 4.10-4.06 (m, 1H), 3.89-3.85 (m, 3H), 3.75-3.72 (m, 2H), 3.57 (dd, J=2.00, 9.40 Hz, 1H), 1.51 (d, J=6.80 Hz, 3H). 19F-NMR (376. MHz, DMSO-d6): δ −132.801. Single isomer with SFC purity=100% (LUX Amylose-1_0.5% IPAm in IPA tR=4.63 min)

Compound 60: LC-MS: Calculated for C₂₅H₂₆FN₃O₄ is 451.49, Observed: 452.3 [M+1]⁺. 1H-NMR (400 MHz, DMSO-d6): δ 7.65 (dd, J=2.00, 6.60 Hz, 2H), 7.49-7.40 (m, 4H), 7.35 (app d, J=1.60 Hz, 1H), 6.93 (t, J=9.20 Hz, 1H), 6.84 (app d, J=1.20 Hz, 1H), 5.68 (m, 2H, 1H exchanges with D2O), 5.53 (t, 1H, exchanges with D2O), 5.37 (d, J=5.60 Hz, 1H, exchanges with D2O), 5.32 (d, J=4.00 Hz, 1H, exchanges with D2O), 4.94-4.92 (m, 1H), 4.14-4.12 (m, 1H), 4.10-4.06 (m, 1H), 3.89-3.85 (m, 3H), 3.75-3.72 (m, 2H), 3.57 (dd, J=2.00, 9.20 Hz, 1H), 1.51 (d, J=6.40 Hz, 3H). 19F-NMR (376. MHz, DMSO-d6): δ −132.81. diastereomeric ratio: 99.5:0.5 (LUX Amylose-1_0.5% IPAm in IPA tR=6.07 min).

### Example A15: Synthesis of Compounds 61 and 62

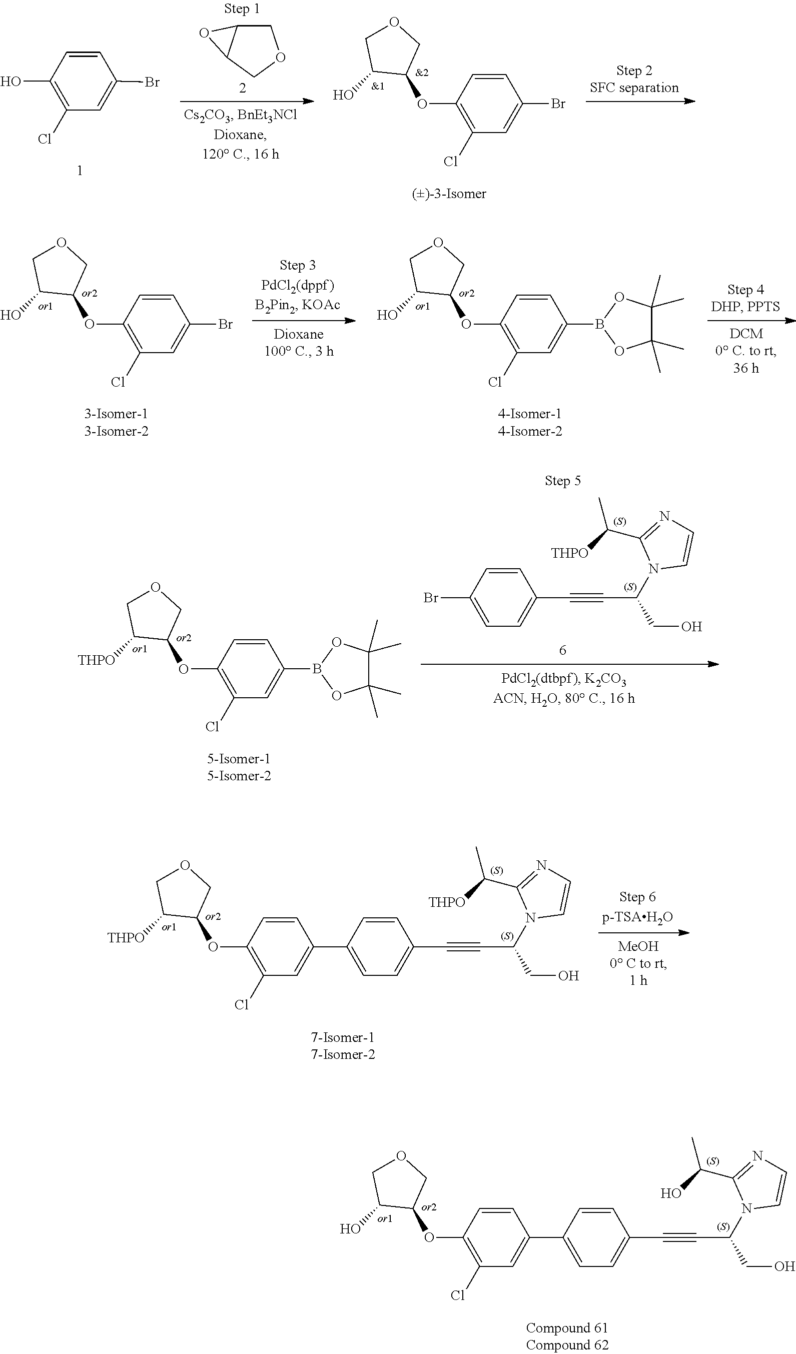

### Steps-1 & 2:

To a stirred solution of 4-bromo-2-chlorophenol (1, 8 g, 38.6 mmol) in 1,4-dioxane (80 mL), were added 3,4-epoxytetrahydrofuran (2, 5.53 mL, 77 mmol), cesium carbonate (18.85 g, 57.8 mmol) and benzyltriethylammonium chloride (1.75 g, 7.71 mmol) at room temperature. The reaction mixture was stirred at 120° C. for 16 h. The inorganic solids were filtered through Celite pad, washed with EtOAc (2×100 mL). The filtrate was concentrated under reduced pressure to afford crude residue. The crude residue was purified by MPLC (manually packed cartridge; SiO2 100-200 mesh; 30% EtOAc in hexanes) to afford (±)-3 as pale-yellow liquid. Yield=7 g (56%). One more batch was carried out on 8 g of 4-bromo-3-chlorophenol (1) to give 8.0 g of (±)-3. Both the batches were mixed and isomers separated by SFC (Column: Chiral Pak ASH-(250*30) mm, 5 μm; Eluents: CO2:0.2% formic acid in Isopropyl alcohol:ACN [85:15]). The fractions were concentrated under reduced pressure to afford 3-Isomer-1 (t_{R}=3.54 min) and 3-Isomer-2 (t_{R}=4.57 min). Yield=3-Isomer-1: 5 g (ee=100%) and 3-Isomer-2: 4.5 g (ee=99%). Both isomers were taken individually for further conversion.

### Step-3:

To a solution of 3-Isomer-1 (3 g, 10.22 mmol) in dioxane (30 mL), were added potassium acetate (3.01 g, 30.7 mmol) and bis(pinacolato)diboron (3.89 g, 15.33 mmol) at room temperature. The resulting mixture was purged with nitrogen for 10 min. To this reaction mixture, PdCl2(dppf) (0.374 g, 0.511 mmol) was added and purged for another 2 min. The reaction mixture was stirred at 100° C. for 3 h. The inorganic solids were filtered through Celite pad and washed with EtOAc (100 mL). The filtrate was concentrated under reduced pressure to afford crude residue. The crude residue was purified by MPLC (manually packed SiO2 cartridge, 100-200 mesh size; 20% EtOAc in hexanes) to afford 4-Isomer-1 as pale-yellow solid. Yield=3.4 g (88%).

### Step-4:

To a solution of 4-Isomer-1 (5 g, 14.68 mmol) in DCM (50 mL), were added DHP (2 mL, 22.02 mmol) and PPTS (0.369 g, 1.468 mmol) at room temperature. The resulting reaction mixture was stirred at room temperature for 36 h. The reaction was quenched with saturated solution of sodium bicarbonate (25 mL) and extracted with DCM (50 mL×2). The combined organic layer was washed with brine (25 mL), dried over anhydrous Na₂SO₄, filtered and concentrated under reduced pressure. The resulting crude residue was purified by MPLC (manually packed SiO2 cartridge, 100-200 mesh size; 15% EtOAc in hexanes) to afford boronate 5-Isomer-1 as a colorless liquid. Yield=4 g (59%).

### Step-5:

To a solution of 6 (2 g, 4.77 mmol) in acetonitrile (15 mL) and water (15 mL), were added boronate ester 5-Isomer-1 (3.04 g, 7.15 mmol) and potassium carbonate (1.978 g, 14.31 mmol) at room temperature. The resulting mixture was purged with nitrogen for 5 min. To this reaction mixture, PdCl2(dtbpf) (0.311 g, 0.477 mmol) was added and the purging continued for 2 min. The reaction mixture was stirred at 80° C. for 16 h after which the reaction was quenched with water (20 mL) and extracted with 10% MeOH in DCM (50 mL×2). The combined organic layer was washed with brine (20 mL), dried over anhydrous Na₂SO₄, filtered and concentrated under reduced pressure. The resulting crude was purified by MPLC (manually packed SiO2 cartridge; 230-400 mesh size; 5% MeOH in DCM) to afford 7-Isomer-1 as pale-brown solid. Yield: 1.1 g (34%). LC-MS: Calculated for C35H41ClN2O7 is 637.17, Observed: 637.2 [M]⁺ and 638.2 [M+1]⁺.

### Step-6:

To a stirred solution of 7-Isomer-1 (200 mg, 0.314 mmol) in MeOH (5 mL), was added p-toluene sulfonic acid monohydrate (179 mg, 0.942 mmol) at 0° C. and the reaction mixture stirred at room temperature for 1 h. The reaction mixture was quenched with sodium bicarbonate solution (20 mL) and extracted with 10% MeOH in DCM (40 mL×2). The combined organic layer was dried over anhydrous Na₂SO₄, filtered and concentrated under reduced pressure to afford the crude residue. The resulting crude residue was purified by MPLC (manually packed SiO2 cartridge, 230-400 mesh size; 5% MeOH in DCM) to afford Compound 61 as pale-brown solid. Yield: 35 mg (23%). LC-MS: Calculated for C25H25ClN2O5 is 468.93; Observed: 469.0 [M+1]⁺. 1H-NMR (400 MHz, DMSO-d6): δ 7.81 (d, J=2.40 Hz, 1H), 7.72 (dd, J=2.00, 6.40 Hz, 2H), 7.68 (dd, J=2.40, 8.80 Hz, 1H), 7.53 (dd, J=2.00, 6.60 Hz, 2H), 7.38 (s, 1H), 7.35 (d, J=8.80 Hz, 1H), 6.87 (s, 1H), 5.68 (t, J=6.00 Hz, 1H), 5.56-5.53 (m, 2H, exchange with D2O), 5.40 (d, J=5.20 Hz, 1H, exchanges with D2O), 4.97-4.94 (m, 1H), 4.82-4.81 (m, 1H), 4.25 (br s, 1H), 4.10 (dd, J=4.00, 10.40 Hz, 1H), 3.95 (dd, J=4.40, 9.20 Hz, 1H), 3.87 (t, J=6.00 Hz, 2H), 3.82 (d, J=10.00 Hz, 1H), 3.63 (dd, J=2.00, 9.20 Hz, 1H), 1.51 (t, J=6.80 Hz, 3H). Single isomer with unknown stereochemistry at tail; SFC purity=95.2% (1-cellulose Z_0.5% IPAm in MeOH; t_{R}=12.37 min).

Compound 62 was synthesized by following the same procedure from step-3 to step-6 of Compound 61. 1H-NMR (400 MHz, DMSO-d6): δ 7.81 (d, J=2.40 Hz, 1H), 7.72 (d, J=8.40 Hz, 2H), 7.67 (dd, J=2.40, 8.80 Hz, 1H), 7.53 (d, J=8.40 Hz, 2H), 7.36 (s, 1H), 7.36-7.34 (m, 1H), 6.84 (app d, J=1.20 Hz, 1H), 5.68 (t, J=5.60 Hz, 1H), 5.56-5.52 (m, 2H, exchange with D2O), 5.37 (d, J=5.60 Hz, 1H, exchanges with D2O), 4.97-4.91 (m, 1H), 4.81 (app d, J=4.00 Hz, 1H), 4.25 (br s, 1H), 4.10 (dd, J=4.00, 10.40 Hz, 1H), 3.95 (dd, J=4.40, 9.20 Hz, 1H), 3.89-3.81 (m, 3H), 3.63 (dd, J=2.00, 9.40 Hz, 1H), 1.51 (t, J=6.40 Hz, 3H). Single isomer with unknown stereochemistry at tail; SFC purity=94.5% (l-cellulose Z_0.5% IPAm in MeOH; t_{R}=13.41 min).

### Example A16: Synthesis of Compound 63

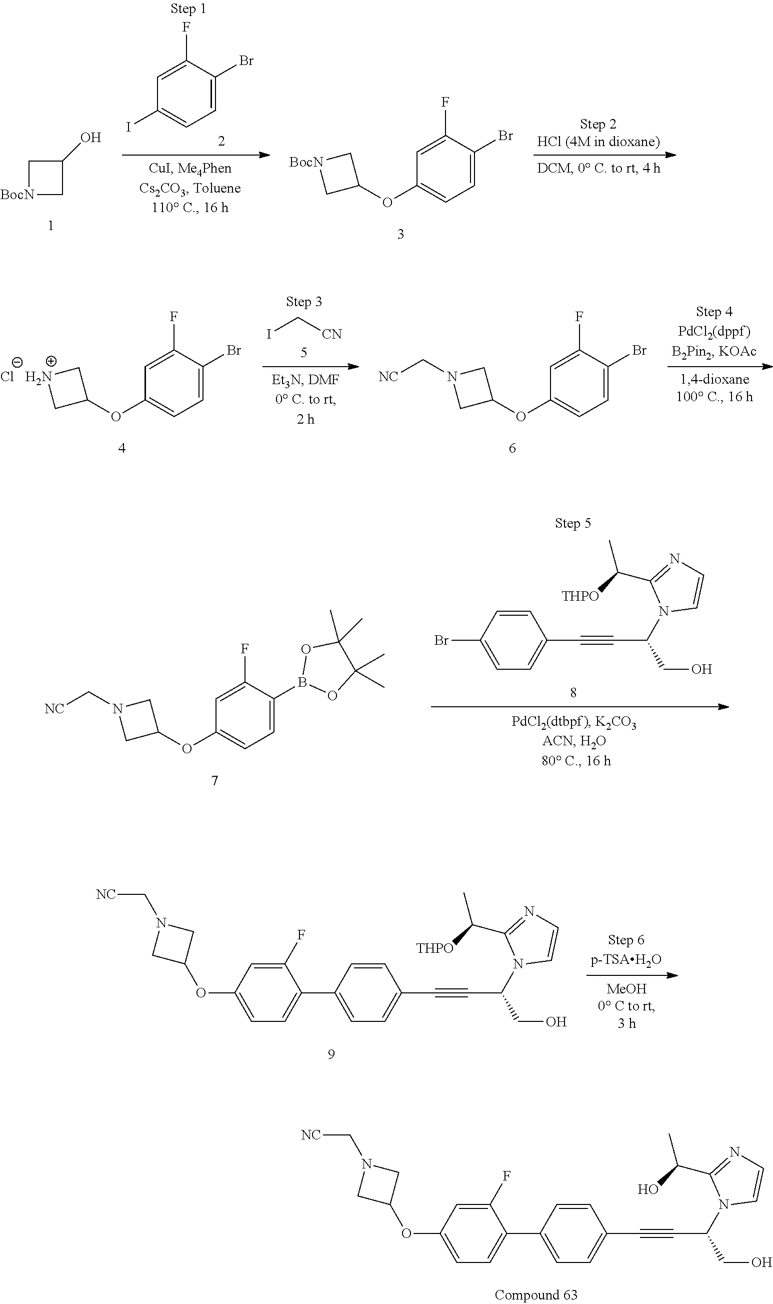

### Step-1:

To a solution 3,4,7,8-tetramethyl-1,10-phenanthroline (Me4Phen, 0.471 g, 1.99 mmol) in toluene (50 mL), was added copper(I) iodide (0.190 g, 0.99 mmol) at room temperature. The resulting mixture was purged with nitrogen for 5 min. To this reaction mixture, tert-butyl 3-hydroxyazetidine-1-carboxylate (1, 3.80 g, 21.93 mmol), 1-bromo-2-fluoro-4-iodobenzene (2, 6.0 g, 19.94 mmol) and cesium carbonate (12.99 g, 39.9 mmol) were added at room temperature. The resulting mixture purged with nitrogen for 5 min and stirred at 110° C. for 16 h. The reaction was cooled to room temperature, filtered through a pad of Celite, washed with EtOAc (200 mL). The filtrate was concentrated, and the resulting crude residue purified by MPLC (using manually packed SiO₂ cartridge, 230-400 mesh size; 18% EtOAc in hexanes) to obtain 3 as white solid. Yield=5 g (92%). LCMS: Calculated for C14H17BrFNO3 is 346.19; Observed: 246.2 [M-Boc]⁺ and 248.0 [M-Boc+2]⁺.

### Step-2:

To a stirred solution of the 3 (5 g, 14.45 mmol) in dioxane (40 mL), was added HCl (4.0 M in dioxane, 28.9 mL, 115.6 mmol) at 0° C. and the reaction mixture stirred at room temperature for 4 h. The volatiles were removed under reduced pressure; the crude residue was co-distilled with toluene (100 mL×2). The solid obtained was triturated with 50% EtOAc in hexanes (100 mL). The solid was filtered and dried in vacuum to afford 4 as an off-white solid. Yield=3.6 g (89%). LCMS: Calculated for C9H10BrFNO⁺: 245.99; Observed: 246.0 [M]⁺ and 248.0 [M+2]⁺.

### Step-3:

To a solution of 4 (3.4 g, 12.03 mmol) in DMF (25 mL), were added triethylamine (5.03 mL, 36.1 mmol) and iodoacetonitrile (5, 1.05 mL, 14.44 mmol) at 0° C. The resulting reaction mixture was stirred at room temperature for 2 h. The reaction was quenched with ice cold water (100 mL) and the solid that precipitated was filtered. This was further washed with hexanes and dried to afford 6 as an off-white solid. Yield=2.9 g (84%). LCMS: Calculated for C11H10BrFN2O is 285.12; Observed: 284.8 [M]⁺ and 286.8 [M+2]⁺.

### Step-4:

To a stirred solution of the 6 (2.9 g, 10.17 mmol) in dioxane (50 mL), were added potassium acetate (2.99 g, 30.5 mmol) and bis(pinacolato)diboron (3.87 g, 15.26 mmol) at room temperature. The resulting mixture was purged with nitrogen for 5 min. To this reaction mixture, PdCl₂(dppf) (0.415 g, 0.50 mmol) was added and stirred at 100° C. for 16 h. The reaction mixture was cooled to room temperature, filtered through Celite pad and washed with EtOAc (200 mL). The filtrate was washed with water (250 mL). The layers were separated, and the aqueous layer extracted with EtOAc (2×250 mL). The combined organic layer was washed with brine (200 mL), dried over anhydrous Na₂SO₄, filtered and concentrated under reduced pressure. The crude residue was purified by MPLC (using manually packed SiO₂ cartridge 230-400 mesh size; 25% EtOAc in hexanes) to obtain 7 as an off-white solid. Yield=2.9 g (71%). LCMS: Calculated for C17H22BFN2O3 is 332.18; Observed: 333.4 [M+1]⁺.

### Step-5:

To a solution of 8 (0.5 g, 1.43 mmol) in acetonitrile (8 mL) and water (3 mL), were added 7 (0.59 g, 1.78 mmol) and K₂CO₃ (0.49 g, 3.58 mmol) at room temperature. The resulting mixture was purged with nitrogen for 10 min. To this reaction mixture, PdCl₂(dtbpf) (0.078 g, 0.12 mmol) was added and stirred at 80° C. for 16 h. The reaction mixture was then quenched with water (20 mL) and extracted with 10% MeOH in DCM (50 mL×2). The combined organic layer was washed with brine (20 mL) and dried over anhydrous Na₂SO₄, filtered and concentrated under reduced pressure. The resulting crude residue was purified by MPLC (using manually packed SiO₂ cartridge 230-400 mesh size; 5% MeOH in DCM) to obtain 9 as pale-brown solid. Yield: 320 mg (47%). LCMS: Calculated for C31H33FN4O4 is 544.62; Observed: 545.2 [M+1]⁺.

### Step-6:

To a stirred solution of 9 (0.31 g, 0.57 mmol) in MeOH (10 mL), was added p-toluenesulfonic acid monohydrate (0.32 g, 1.70 mmol) at 0° C. and the reaction mixture stirred at room temperature for 3 h. The volatiles were then removed under reduced pressure. The resulting residue was dissolved in 10% MeOH in DCM (100 mL) and washed with 10% NaHCO3 solution (20 mL). The organic extract was dried over anhydrous Na₂SO₄, filtered and concentrated under reduced pressure. The resulting crude residue was purified by reverse phase prep HPLC (Column: Shimpack C18 (20*150 mm) 5 μm; Eluents: 10 mM ammonium bicarbonate in water and ACN) to afford Compound 63 as white solid. Yield: 55 mg (21%). LCMS: Calculated for C26H25FN4O3 is 460.50; Observed: 461.0 [M+1]⁺. 1H-NMR (400 MHz, DMSO-d6): δ 7.56-7.52 (m, 4H), 7.48 (t, J=8.80 Hz, 1H), 7.35 (d, J=0.80 Hz, 1H), 6.91-6.82 (m, 3H), 5.69 (t, J=6.00 Hz, 1H), 5.54 (t, J=5.20 Hz, 1H, exchanges with D2O), 5.38 (d, J=5.60 Hz, 1H, exchanges with D2O), 4.96-4.91 (m, 2H), 3.88-3.81 (m, 4H), 3.75 (s, 2H), 3.26-3.23 (m, 2H), 1.51 (d, J=6.40 Hz, 3H). 19F-NMR (376. MHz, DMSO-d6): δ −115.21. Single isomer with SFC purity 100% (l-Cellulose-Z_0.5% IPAm in MeOH tR=3.50 min).

### Example A17: Synthesis of Compound 64

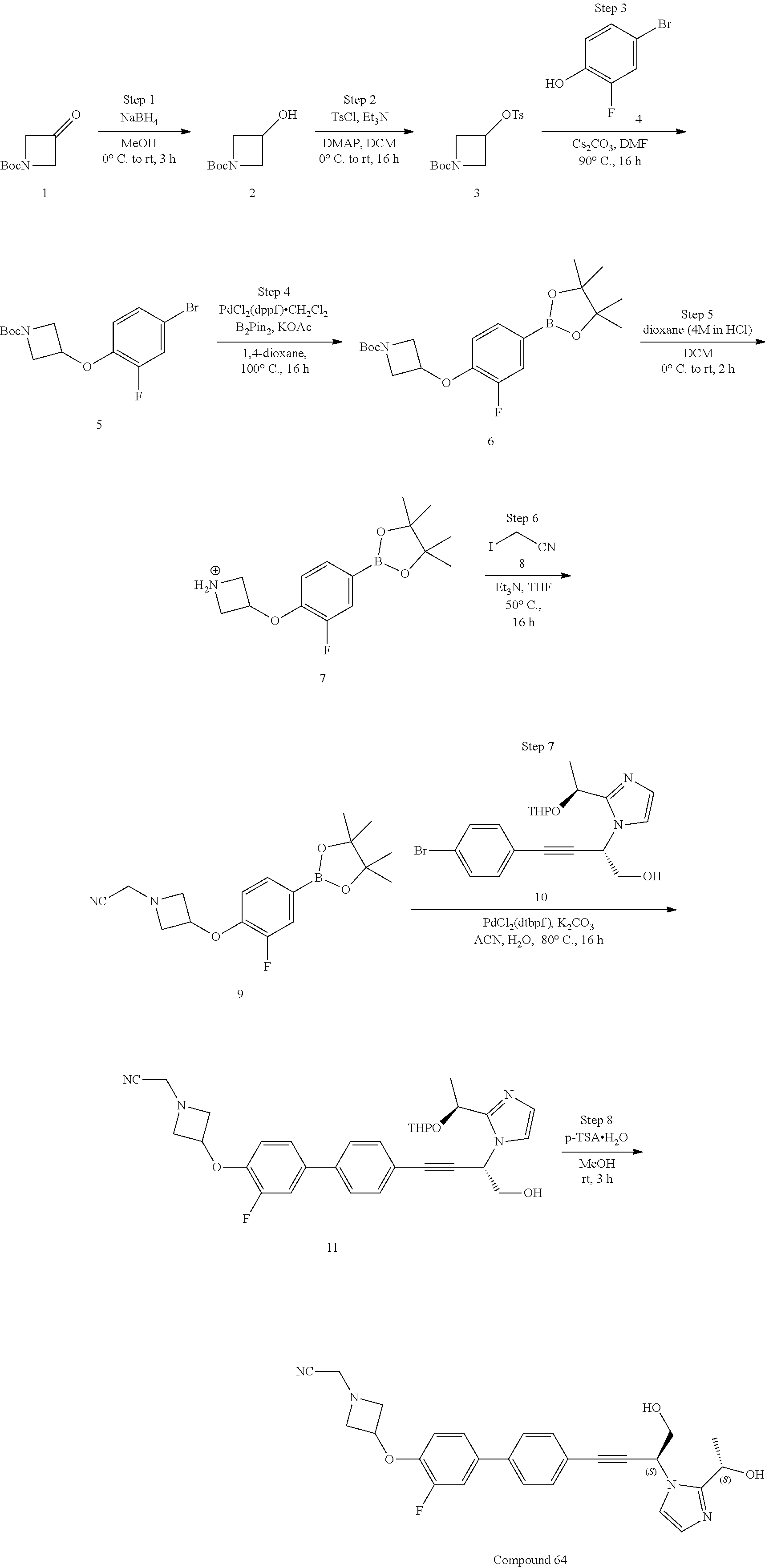

### Step-1:

To a stirred solution of tert-butyl 3-oxoazetidine-1-carboxylate (1, 25 g, 146 mmol) in MeOH (200 mL), was added NaBH₄ (5.52 g, 146 mmol) in portions at 0° C. under nitrogen atmosphere. The reaction mixture was stirred at room temperature for 3 h. After completion of the reaction, the reaction mixture was concentrated under reduced pressure. To the resulting residue, water (200 mL) was added and extracted with EtOAc (2×200 mL). The combined organic layer was dried over anhydrous sodium sulphate, filtered and concentrated reduced pressure to afford 2 as a brown coloured liquid. Yield: 23 g (86%).

### Step-2:

To a stirred solution of 2 (3 g, 17.32 mmol) in DCM (50 mL), were added TEA (7.24 mL, 52.0 mmol), DMAP (212 mg, 1.732 mmol) and tosyl chloride (4.29 g, 22.52 mmol) at 0° C. under nitrogen. The reaction mixture was stirred at room temperature for 16 h. The reaction mixture was quenched with water (50 mL) and extracted with DCM (50 mL×2). The combined organic layer was dried over anhydrous sodium sulphate, filtered and concentrated under reduced pressure. The resulting crude was purified by MPLC (using manually packed SiO₂ cartridge, 100-200 mesh size; 15-20% EtOAc in hexane) to afford 3 as an oily liquid. Yield: 5 g (84%).

### Step-3:

To a solution of 3 (5 g, 15.27 mmol) in DMF (50 mL), were added Cs₂CO₃ (14.93 g, 45.8 mmol) and 4-bromo-2-fluorophenol (4, 1.844 ml, 16.80 mmol) at room temperature under nitrogen, and the resulting reaction mixture stirred at 90° C. for 16 h. The reaction mixture was quenched with water (100 mL) and extracted with EtOAc (2×100 mL). The combined organic layer was dried over anhydrous sodium sulphate, filtered and concentrated reduced pressure. The resulting crude was purified by MPLC (using manually packed SiO₂ cartridge, 100-200 mesh size; 15-20% EtOAc in hexane) to afford 5 as a white solid. Yield: 4.2 g (75%).

### Step-4:

To a stirred solution of 5 (2 g, 5.78 mmol) in 1,4-dioxane (30 mL), were added potassium acetate (1.701 g, 17.33 mmol) and bis(pinacolato)diboron (2.201 g, 8.67 mmol) at room temperature. The resulting mixture was purged with nitrogen for 10 min. To this reaction mixture, PdCl₂(dppf)·CH₂Cl₂ adduct (0.236 g, 0.289 mmol) was added and the purging with nitrogen continued for 2 min. The reaction mixture was stirred at 100° C. for 16 h. The reaction mixture was cooled to room temperature, filtered through a Celite bed. The Celite bed was washed with EtOAc (100 mL). The combined filtrate was washed with brine solution (50 mL), dried over anhydrous sodium sulphate, filtered and concentrated under reduced pressure to get crude residue. The resulting crude mass was purified by MPLC (using manually packed SiO₂ cartridge, 230-400 mesh; 20% EtOAc in hexanes) to afford 6 as a pale-yellow gum. Yield: 2 g (79%).

### Step-5:

To a stirred solution of 6 (2.0 g, 5.09 mmol) in DCM (20 mL), was added HCl (4 M in 1,4 dioxane, 3.81 mL, 15.26 mmol) at 0° C. The reaction mixture was stirred at room temperature for 2 h. The volatiles in the reaction mixture were removed under reduced pressure to afford 7 as a pale-yellow solid. The crude material was taken to the next step without any purification. Yield=1.4 g (78%). LC-MS: Calculated for C₁₅H₂₂BFNO₃⁺ is 294.1, Observed: 294.2 [M]⁺ and 212 [M]⁺ of boronic acid.

### Step-6:

To a solution of 7 (1.3 g, 3.94 mmol) in THF (20 mL), were added triethylamine (3.85 mL, 27.6 mmol) and 2-iodoacetonitrile (8, 0.573 mL, 7.89 mmol) at 0° C. and the resulting reaction mixture stirred at 50° C. for 16 h. The reaction was quenched with water (50 mL). The aqueous layer was extracted with EtOAc (50 mL×2). The combined organic layer was dried over anhydrous sodium sulphate, filtered and concentrated under reduced pressure to get crude residue. The resulting crude mass was purified by MPLC (using manually packed SiO₂ cartridge, 100-200 mesh; 20% EtOAc in hexanes) to afford 9 as a pale-yellow solid. LC-MS: Calculated C₁₇H₂₂BFN₂O₃ is 332.1, Observed: 333.0 [M+1]⁺ and 251.0 [M+1]⁺ of boronic acid. Yield: 800 mg (57%).

### Step-7:

To a stirred solution of 10 (600 mg, 1.431 mmol) in acetonitrile (10 mL) and water (2.5 mL), were added 9 (570 mg, 1.717 mmol) and K₂CO₃ (593 mg, 4.29 mmol) and the reaction mixture purged with nitrogen for 5 min. To this reaction mixture, PdCl₂(dtbpf) (93 mg, 0.143 mmol) was added and the purging continued with nitrogen for another 2 min. The reaction mixture was stirred at 80° C. for 16 h. The reaction mixture was quenched with water (50 mL), extracted with 10% MeOH in DCM (50 mL×2). The combined organic layer was washed with brine solution (30 mL), dried over anhydrous sodium sulphate, filtered and concentrated under reduced pressure. The resulting crude product was purified by MPLC (using manually packed SiO₂ cartridge, 230-400 mesh size; 5% MeOH in DCM) to afford 11 as a pale-brown solid. Yield: 250 mg (31%). LC-MS: Calculated for C₃₁H₃₃FN₄O₄ is 544.6, Observed: 545.3 [M+1]⁺.

### Step-8:

To a stirred solution of 11 (250 mg, 0.459 mmol) in MeOH (10 mL), was added p-toluene sulfonic acid monohydrate (262 mg, 1.377 mmol) at 0° C. The reaction mixture was stirred at room temperature for 3 h. The volatiles were evaporated under reduced pressure, the resulting residue was basified with 10% NaHCO₃ solution (30 mL) and extracted with 10% MeOH in DCM (50 mL×2). The combined organic layer was washed with brine (30 mL), dried over anhydrous sodium sulphate, filtered and concentrated under reduced pressure. The crude compound was purified by SFC purification (Column: CHIRALPAK-IH-(250*30) mm, 5 μm Eluents: CO₂:0.5% isopropyl amine in isopropanol [55:45]) and lyophilized to obtained Compound 64 as a white solid. Yield=50 mg (23%). LC-MS: Calculated for C₂₆H₂₅FN₄O₃ is 460.5, Observed: 461.2 [M+1]⁺. 1H-NMR (400 MHz, DMSO-d6): δ 7.71-7.69 (m, 2H), 7.66 (dd, J=2.40, 12.80 Hz, 1H), 7.53 (dd, J=1.60, 6.60 Hz, 2H), 7.49-7.46 (m, 1H), 7.36 (app d, J=1.60 Hz, 1H), 7.08 (t, J=8.80 Hz, 1H), 6.84 (app d, J 1.20 Hz, 1H), 5.69 (t, 1H), 5.53 (t, 1H, exchanges with D2O), 5.37 (d, J=5.60 Hz, 1H, exchanges with D2O), 4.95-4.94 (m, 2H), 3.88-3.81 (m, 4H), 3.76 (s, 2H), 3.30-3.27 (m, 2H, merges with solvent water), 1.51 (d, J=6.40 Hz, 3H). 19F-NMR (376. MHz, DMSO-d6): δ −133.94. CHIRALPAK-IH_0.5% IPAmin IPA tR=5.30 min. Single isomer with SFC purity=100%.

### Example A18: Synthesis of Compound 65

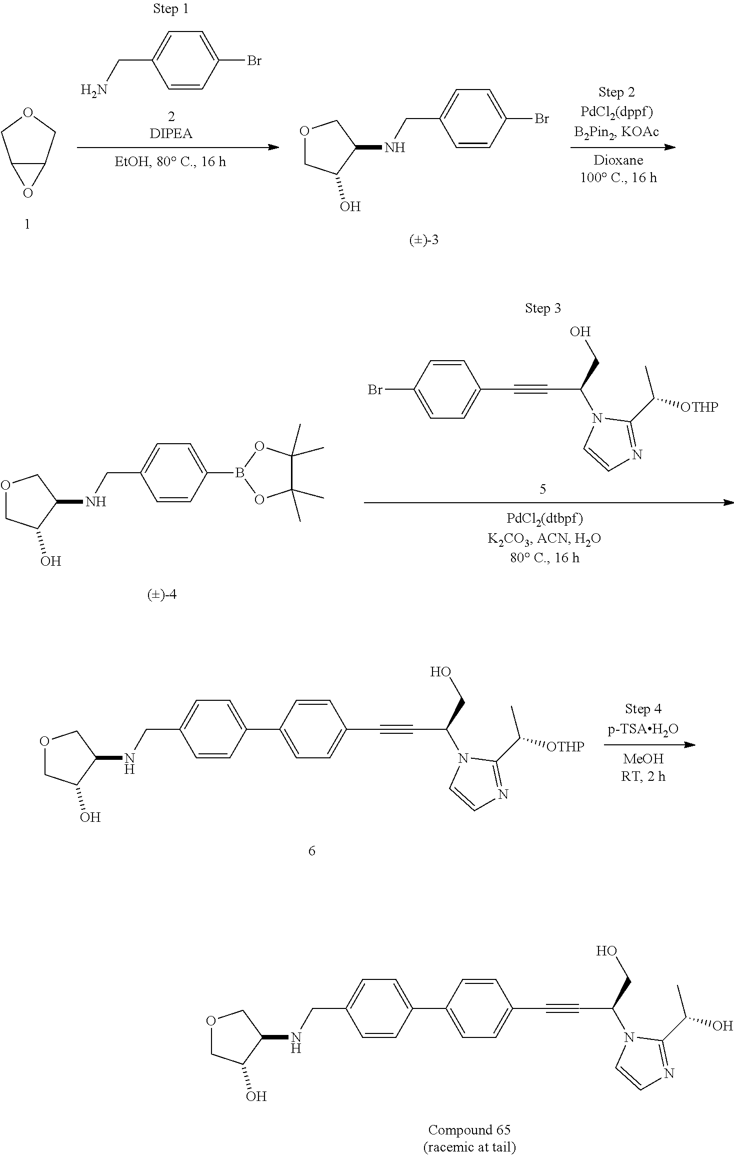

### Step-1:

To a stirred solution of 3,4-epoxytetrahydrofuran (1, 6.94 g, 81 mmol) in EtOH (50 mL), were added 4-Bromobenzylamine (2, 5 g, 26.9 mmol) and DIPEA (23.47 mL, 134 mmol) at room temperature. The reaction mixture was stirred at 80° C. for 16 h. The reaction mixture was concentrated under reduced pressure, quenched with water (50 mL), and extracted with DCM (50 mL×2). The combined organic layer was washed with brine (30 mL), dried over anhydrous Na₂SO₄, filtered and concentrated under reduced pressure. The crude residue was purified by using MPLC (using manually packed SiO₂ cartridge, 230-400 mesh; 40% acetonitrile in DCM) to afford (±)-3 as a pale-yellow solid. Yield: 2.1 g (28%). UPLC-MS: Calculated for C11H14BrNO2 is 272.142, Observed: 272.1 [M]⁺ and 274.1 [M+2]⁺.

### Step-2:

To a stirred solution of (±)-3 (2 g, 7.35 mmol) and bis(pinacolato)diboron (2.80 g, 11.02 mmol) in 1,4-dioxane (30 mL), was added potassium acetate (2.164 g, 22.05 mmol) at room temperature. The reaction mixture was degassed for 5 min after which PdCl2(dppf) (0.538 g, 0.735 mmol) was added and the purging continued for another 2 min and heated at 100° C. for 16 h. The reaction mixture was concentrated under reduced pressure, the residue taken in water (100 mL) and extracted with DCM (50 mL×2). The combined organic layer was washed with brine (50 mL), dried over anhydrous Na₂SO₄, filtered and concentrated under reduced pressure to give the crude material. The crude residue was purified by MPLC (using manually packed SiO₂ cartridge, 230-400 mesh; 10% MeOH in DCM) to afford (±)-4 as a brown oil. Yield: 1 g (40%). UPLC-MS: Calculated for C17H26BNO4 is 319.2, Observed: 320.3 [M+1]⁺.

### Step-3:

To a stirred solution of 5 (0.900 g, 2.14 mmol) in acetonitrile (20 mL) and water (20 mL), were added (±)-4 (1.0 g, 3.22 mmol) and K2CO3 (0.890 g, 6.44 mmol) at room temperature. The resulting mixture was purged with nitrogen for 5 min. To this reaction mixture, PdCl2(dtbpf) (0.140 g, 0.215 mmol) was added and the purging continued for another 2 min. The resulting reaction was stirred at 80° C. for 16 h. The reaction mixture, as monitored by TLC, showed the formation of polar spot along with starting materials. The reaction mixture was cooled to room temperature and purged with N₂ for 5 min. To this reaction mixture, another lot of PdCl2(dtbpf) (0.140 g, 0.215 mmol) was added and the purging continued for another 2 min. The resulting reaction was stirred at 80° C. for 16 h. The reaction mixture was quenched with water (50 mL) and extracted with 20% MeOH in DCM (30 mL×2). The combined organic layer was washed with brine (20 mL), dried over anhydrous Na₂SO₄, filtered and concentrated under reduced pressure. The crude product was purified by using MPLC (SiO₂ 230-400 mesh; 10% MeOH in DCM) to afford 6 as a brown solid. Yield: 1 g (66%). UPLC-MS: Calculated for C31H37N3O5 is 531.6, Observed: 532.2 [M+1]⁺.

### Step-4:

To a stirred solution of 6 (1.0 g, 1.881 mmol) in MeOH (25 mL), was added p-toluenesulfonic acid monohydrate (1.073 g, 5.64 mmol) at room temperature and the resulting reaction mixture stirred at room temperature for 2 h. The volatiles in the reaction were concentrated under reduced pressure, the crude residue was basified with sat. NaHCO₃ solution (50 mL). The aqueous layer was extracted with 20% MeOH in DCM (30 mL×2). The combined organic extract was dried over anhydrous Na₂SO₄, filtered and concentrated to afford crude mass. The crude residue was purified by preparative HPLC (Column: X-BRIDGE C8 (19×150 mm) 5 μm; Eluents:10 mM ammonium bicarbonate in water and ACN) to obtain 90 mg as an off-white solid. The resulting compound was purified by SFC purification (Column: I CELLULOSE B (250×30) mm, 5 μm; Eluents: CO2:0.5% isopropylamine in MeOH (75:25)) to obtain Compound 65 as an off-white solid. Yield: 60 mg (7%). UPLC-MS: Calculated for C26H29N3O4 is 447.5, Observed: 448.8 [M+1]⁺. The product is a mixture of diastereomers (racemic at tail).

### Example A19: Synthesis of Compound 66

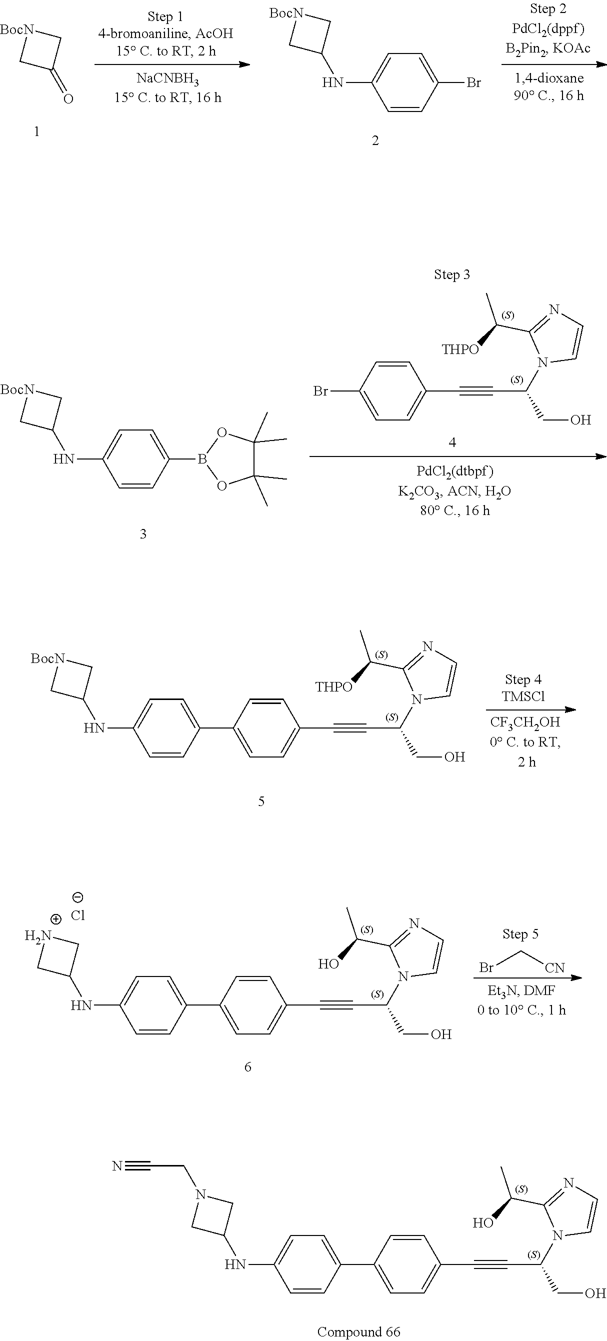

### Step-1:

To a stirred solution of 1-Boc-3-azetidinone (1, 5.97 g, 34.9 mmol) in AcOH (100 mL), was added 4-bromoaniline (5 g, 29.1 mmol) at 15° C., and the reaction mixture stirred further at 25° C. for 2 h. To the reaction mixture cooled to 15° C., sodium cyanoborohydride (5.48 g, 87 mmol) was added in portions over a period of 30 min. After complete addition, the resulting reaction mixture was stirred at room temperature for 16 h. The reaction mixture was quenched with ice cold water (500 mL) and stirred for 10 min. The separated solid was filtered, washed with ice cold water (120 mL), and dried under reduced pressure to afford 2 as an off-white solid. Yield: 7 g (66%).

### Step-2:

To a stirred solution of 2 (7 g, 21.39 mmol) in 1,4-dioxane (100 mL), were added bis(pinacolato)diboron (8.15, 32.1 mmol) and potassium acetate (6.30 g, 64.2 mmol) at room temperature and the reaction mixture purged with nitrogen for 15 min. To the reaction mixture, PdCl2(dppf) (1.565 g, 2.139 mmol) was added, and the reaction mixture heated to 90° C. and for 16 h. The reaction mixture was cooled to 25° C. and filtered through a Celite pad. The pad was washed with EtOAc (50 mL), the filtrate combined and concentrated under reduced pressure. The crude residue, thus obtained, was purified by using MPLC (manually packed SiO2 cartridge; 100-200 mesh size; 30% EtOAc in hexanes) to afford 3 as a pale-yellow solid. Yield: 4.8 g (59%). LC-MS: Calculated for C20H31BN2O4 is 374.24, Observed: 319.3 [(M-t-Bu)+1]⁺ and 275.3 [(M-Boc)+1]⁺.

### Step-3:

To a stirred solution of 3 (0.536 g, 1.431 mmol) in acetonitrile (7 mL) and water (3 mL), were added 4 (0.5 g, 1.192 mmol) and potassium carbonate (0.494 g, 3.58 mmol) at 25° C. After bubbling nitrogen through the reaction mixture for 15 min, PdCl2(dtpbf) (0.078 g, 0.119 mmol) was added and the reaction mixture stirred at 80° C. for 16 h. The reaction mixture was cooled to 25° C., diluted with EtOAc (20 mL), filtered through the Celite pad. The pad was washed with EtOAc (20 mL), the filtrate combined and concentrated under reduced pressure. The resulting crude residue was further dissolved in 10% MeOH in DCM (10 mL), washed with water (3 mL), followed by brine solution (2 mL), dried over anhydrous Na2SO4, filtered and concentrated under reduced pressure. The crude residue, thus obtained, was purified by using MPLC (manually packed SiO₂ cartridge, 100-200 mesh, 10% MeOH in EtOAc) to afford 5 as a pale-brown gum. Yield: 500 mg (63%). LC-MS: Calculated for C34H42N4O5 is 586.73, Observed: 587.5 [M+1]⁺.

### Step-4:

To a stirred solution of 5 (500 mg, 0.852 mmol) in trifluoroethanol (10 mL), was added TMSCl (0.327 mL, 2.56 mmol) at 0° C. and the reaction mixture stirred at 25° C. for 2 h. The reaction mixture was concentrated under reduced pressure to afford 6 as a pale-brown gum. Yield: 350 mg (74%). LC-MS: Calculated for C24H27N4O2⁺ is 403.51, Observed: 402.9 [M]⁺.

### Step-5:

To a stirred solution of 6 (0.35 g, 0.797 mmol) in DMF (3 mL), was added triethylamine (0.556 mL, 3.99 mmol) at 0° C. and stirred for 10 min. To this reaction mixture, bromoacetonitrile (0.083 mL, 1.196 mmol) was added at 0° C. and the resulting reaction mixture stirred for 1 h at 10° C. The reaction mixture was quenched with ice cold water (15 mL), extracted with EtOAc (3×10 mL). The combined organic extract was washed with ice cold water (5 mL), brine solution (5 mL), dried over anhydrous Na2SO4, filtered and concentrated under reduced pressure. The crude residue, thus obtained, was purified by preparative HPLC (Column: X-BRIDGE C18 (19×150 nm) 5 μm; Eluents: 10 mM ammonium bicarbonate in water and acetonitrile) to afford Compound 66 as an off-white solid. Yield: 90 mg (25%). LC-MS: Calculated for C26H27N5O2 is 441.54, Observed: 442.4 [M+1]⁺. 1H-NMR (400 MHz, DMSO-d6): δ 7.58 (d, J=8.40 Hz, 2H), 7.48-7.45 (m, 4H), 7.35 (d, J=0.80 Hz, 1H), 6.84 (d, J=0.80 Hz, 1H), 6.61 (d, J=8.80 Hz, 2H), 6.43 (d, J=6.40 Hz, 1H, exchanges with D2O), 6.05 (br s, 1H, exchanges with D2O), 5.68 (t, J=6.00 Hz, 1H), 5.37 (br s, 1H, exchanges with D2O), 4.94-4.91 (m, 1H), 4.05-4.03 (m, 1H), 3.86 (d, J=6.00 Hz, 2H), 3.72-3.70 (m, 4H), 3.06 (t, J=7.60 Hz, 2H), 1.51 (d, J=6.40 Hz, 3H). SFC: 98.6%; tR=3.66 min (Column: Whelk-(R,R); Mobile phase: CO2 and 0.5% isopropyl amine in MeOH). Single isomer with SFC purity=98.6%.

### Example A20: Synthesis of Compound 67

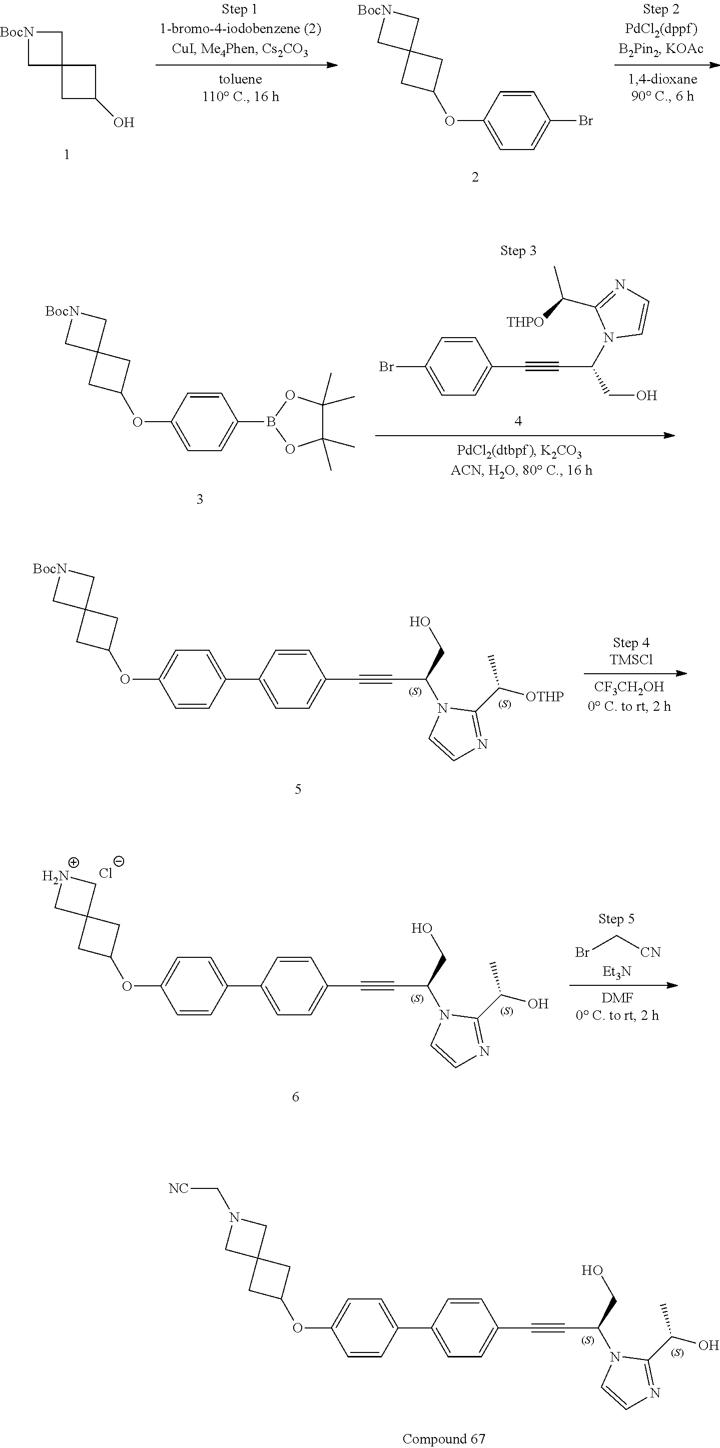

### Step 1:

A solution of copper(I) iodide (0.135 g, 0.707 mmol) and 3,4,7,8-tetramethyl-1,10-phenanthroline (Me4Phen, 0.334 g, 1.414 mmol) in toluene (60 mL) was purged with nitrogen for 5 min. To this reaction mixture, cesium carbonate (9.21 g, 28.3 mmol), tert-butyl 6-hydroxy-2-azaspiro[3.3]heptane-2-carboxylate (1, 3.32 g, 15.55 mmol) and 1-bromo-4-iodobenzene (4 g, 14.14 mmol) were added and the purging continued for 5 min. The reaction mixture was stirred at 110° C. for 16 h. The reaction mixture was filtered through a Celite pad and washed with EtOAc (150 mL). The combined filtrate was concentrated under reduced pressure. The crude residue, thus obtained, was purified by using MPLC (manually packed SiO₂ cartridge, 100-200 mesh size; 9% EtOAc in hexane) to obtain 2 as an off-white solid. Yield: 3 g (46%). LCMS: Calculated for C₁₇H₂₂BrNO₃ is 368.27, Observed: 268.1 [M-Boc]⁺ and 270.2 [(M-Boc)+2]⁺.

### Step 2:

To a stirred solution of 2 (4.8 g, 13.03 mmol) and bis(pinacolato)diboron (6.62 g, 26.1 mmol) in 1,4-dioxane (100 mL), was added potassium acetate (5.12 g, 52.1 mmol). The reaction mixture was purged for 5 min with nitrogen. To this reaction mixture PdCl₂(dppf) (0.954 g, 1.303 mmol) was added and purging continued for 2 min. The reaction mixture was stirred at 90° C. for 6 h. The reaction mixture was filtered through the Celite pad and washed with EtOAc (150 mL) and concentrated under reduced pressure. The resulting crude residue was purified by using MPLC (manually packed SiO₂ cartridge, 230-400 mesh size; 20% EtOAc in hexanes) to obtain 3 as an off-white solid. Yield: 3 g (52%). LCMS: Calculated for C₂₃H₃₄BNO₅ is 415.33, Observed: 316.2 [(M-Boc)+1]⁺.

### Step 3:

To a stirred solution of 3 (631 mg, 1.520 mmol) and 4 (500 mg, 1.014 mmol) in a mixture of acetonitrile (8 mL) and water (8 mL), was added K₂CO₃ (420 mg, 3.04 mmol) and the reaction mixture purged with nitrogen for 5 min. To this reaction mixture, PdCl2(dtbpf) (66.1 mg, 0.101 mmol) was added and the purging continued for 2 min. The reaction mixture was stirred at 80° C. for 16 h. The reaction was quenched with water (50 mL) and extracted with 10% MeOH in DCM (2×50 mL). The combined organic layer was washed with water (50 mL), brine (50 mL), dried over anhydrous sodium sulfate, filtered and concentrated under reduced pressure. The crude residue thus obtained was purified by using MPLC (manually packed SiO₂ cartridge, 100-200 mesh size) to obtain 5 as an off-white solid. Yield: 320 mg (48%). LCMS: Calculated for C₃₇H₄₅N₃O₆ is 627.78, Observed: 628.4 [M+1]⁺.

### Step 4:

To a stirred solution of 5 (0.3 g, 0.478 mmol) in 2,2,2 trifluoroethanol (5 mL), was added TMSCl (0.092 mL, 0.717 mmol) at 0° C. The reaction mixture was stirred at room temperature for 2 h. The reaction was concentrated under reduced pressure to obtain 6 as a brown gum. The crude mass was taken to the next step without purification. Yield: 220 mg (82%). LCMS: Calculated for C27H30N3O3⁺ is 444.55, Observed: 444.2 [M]⁺.

### Step 5:

To a stirred solution of 6 (200 mg, 0.417 mmol) in DMF (4 mL), were added Et3N (0.348 mL, 2.500 mmol) and 2-bromoacetonitrile (0.044 ml, 0.625 mmol) at 0°, and the reaction stirred at room temperature for 2 h. The reaction mixture was then quenched with water (50 mL) and extracted with 10% MeOH in DCM (2×50 mL). The combined organic layer was washed with water (50 mL), brine (50 mL), dried over anhydrous sodium sulfate, filtered and concentrated under reduced pressure. The crude residue, thus obtained, was purified by reverse phase column chromatography using MPLC (Column: Redisep, C18 silica gel, Eluents: water and ACN) to obtain Compound 67 as an off-white solid. Yield: 62 mg (30%). LCMS: Calculated for C₂₉H₃₀N₄O₃ is 482.58, Observed: 483.4 [M+1]⁺. 1H-NMR (400 MHz, DMSO-d6): δ 7.65 (d, J=8.80 Hz, 2H), 7.61 (d, J=8.80 Hz, 2H), 7.51 (d, J=8.40 Hz, 2H), 7.35 (app d, J=1.20 Hz, 1H), 6.93 (d, J=8.80 Hz, 2H), 6.84 (app d, J=0.80 Hz, 1H), 5.69 (t, J=6.00 Hz, 1H), 5.53 (t, J=5.20 Hz, 1H, exchanges with D2O), 5.37 (d, J=5.60 Hz, 1H, exchanges with D2O), 4.97-4.91 (m, 1H), 4.71-4.64 (m, 1H), 3.87 (t, J=5.60 Hz, 2H), 3.59 (s, 2H), 3.32 (s, 2H, merges with solvent peak), 3.26 (s, 2H), 2.71-2.66 (m, 2H), 2.20-2.16 (m, 2H), 1.51 (d, J=6.80 Hz, 3H).

SFC purity=99.5% (Column: l-cellulose Z; Eluents: 0.5% isopropyl amine in MeOH and CO2); tR=2.52 min. Single isomer with SFC purity=99.5%

### Example A21: Synthesis of Compound 68

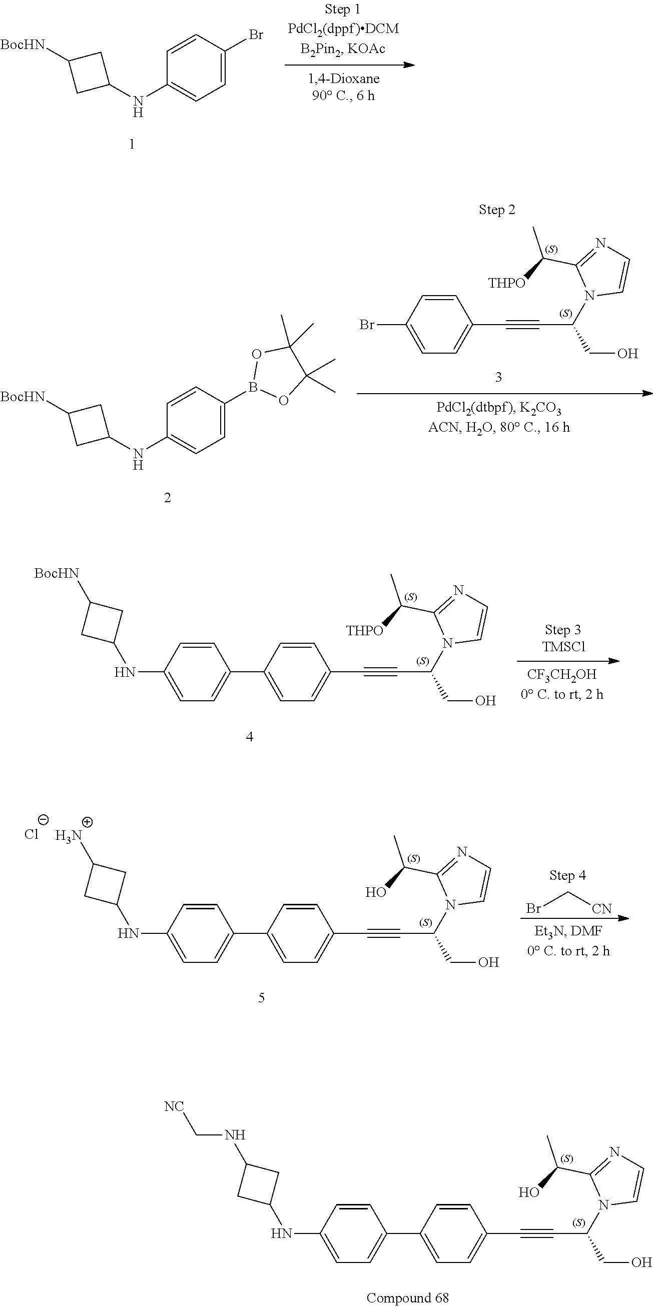

### Step 1:

To a stirred solution of tert-butyl (3-((4-bromophenyl)amino)cyclobutyl)carbamate (1, 2.7 g, 7.91 mmol) and bis(pinacolato)diboron (4.02 g, 15.82 mmol) in 1,4-dioxane (30 mL), was added potassium acetate (3.11 g, 31.6 mmol) and the reaction mixture degassed for 5 min by using nitrogen. To this reaction mixture, PdCl2(dppf)·DCM (0.579 g, 0.791 mmol) was added and the degassing continued for 2 min. The reaction mixture was then stirred at 90° C. for 6 h. The reaction was filtered through the Celite pad, washed with EtOAc (150 mL) and concentrated under reduced pressure. The resulting crude residue was purified by MPLC (using manually packed cartridge; SiO₂ 230-400 mesh; 20% EtOAc in hexane) to obtain 2 as a colorless gum. Yield: 1.4 g (44%). LCMS: Calculated for C₂₁H₃₃BN₂O₄ is 388.25, Observed: 389.4 [M+H]⁺

### Step 2:

To a stirred solution of 2 (1.389 g, 3.58 mmol) and 3 (1 g, 2.385 mmol) in a mixture of acetonitrile (10 mL) and water (10 mL), was added K2CO3 (0.989 g, 7.15 mmol) at room temperature. The reaction mixture was purged for 5 min using nitrogen gas. To this reaction mixture, PdCl2(dtbpf) (0.155 g, 0.238 mmol) was added and the purging continued for 2 min. The reaction mixture was stirred at 80° C. for 16 h. The reaction was quenched with water (100 mL) and extracted with 10% MeOH in DCM (2×100 mL). The combined organic layer was washed with water (100 mL), brine (100 mL), dried over sodium sulfate, filtered and the filtrate concentrated under reduced pressure. The crude residue thus obtained was purified by MPLC (using manually packed cartridge; SiO₂ 230-400 mesh; 6% MeOH in DCM) to obtain 4 as a pale-brown solid. Yield: 700 mg (38%). LCMS: Calculated for C₃₅H₄₄N₄O₅ is 600.33, Observed: 601.2 [M+H]⁺

### Step 3:

To a stirred solution of 4 (500 mg, 0.832 mmol) in 2,2,2 trifluoroethanol (5 mL), was added TMSCl (0.213 mL, 1.665 mmol) at 0° C. under nitrogen atmosphere. The reaction was stirred at room temperature for 2 h. The reaction was concentrated under reduced pressure to obtain brown gum. The crude was taken to the next step without purification. Yield: 400 mg (59%). LCMS: Calculated for C₂₅H₂₉N₄O₂⁺ is 416.53; Observed: 417.4 [M]⁺

### Step 4:

To a stirred solution of 5 (340 mg, 0.751 mmol) in DMF (6 mL) at 0° C. under nitrogen atmosphere were added triethylamine (0.626 mL, 4.50 mmol) and 2-bromoacetonitrile (0.078 mL, 1.126 mmol). The reaction was stirred at room temperature for 2 h. The reaction mixture was quenched with water (40 mL) and extracted with 10% MeOH in DCM (2×40 mL). The combined organic layer was washed with water (40 mL), brine (40 mL), dried over sodium sulfate, filtered and concentrated under reduced pressure. The resulting crude residue was purified by reversed phase preparative HPLC: (Column: Shimpack C18 (150*199 mm) 5 μm; Eluents: 10 mM ammonium bicarbonate in H20 and ACN) to obtain Compound 68 as an off-white solid. Yield: 50 mg (13%). LCMS: Calculated for C₂₇H₂₉N₅O₂ is 455.23, Observed: 455.7 [M+H]⁺. 1H-NMR for the major isomer (400 MHz, DMSO-d6): δ 7.58 (d, J=8.80 Hz, 2H), 7.47-7.44 (m, 4H), 7.35 (app d, J=1.20 Hz, 1H), 6.84 (app d, J=1.20 Hz, 1H), 6.59 (d, J=8.80 Hz, 2H), 6.11 (d, J=6.40 Hz, 1H, exchanges with D2O), 5.68 (t, J=6.00 Hz, 1H), 5.56 (br s, 1H, exchanges with D2O), 5.38 (br s, 1H, exchanges with D2O), 4.96-4.91 (m, 1H), 3.86 (d, J=6.40 Hz, 2H), 3.59-3.53 (m, 3H), 3.04-3.02 (m, 1H), 2.78-2.66 (m, 3H), 1.65-1.57 (m, 2H), 1.51 (d, J=6.40 Hz, 3H). SFC: Column: Lux l-Amylose-3; Mobile phase: 0.5% IPAm in MeOH and ACN; tR=7.08 min (26.7%) and 10.31 min (72.3%). cis and trans mixture at tail; the ratio based on SFC data is 27:72.

### Example A22: Synthesis of Compound 69

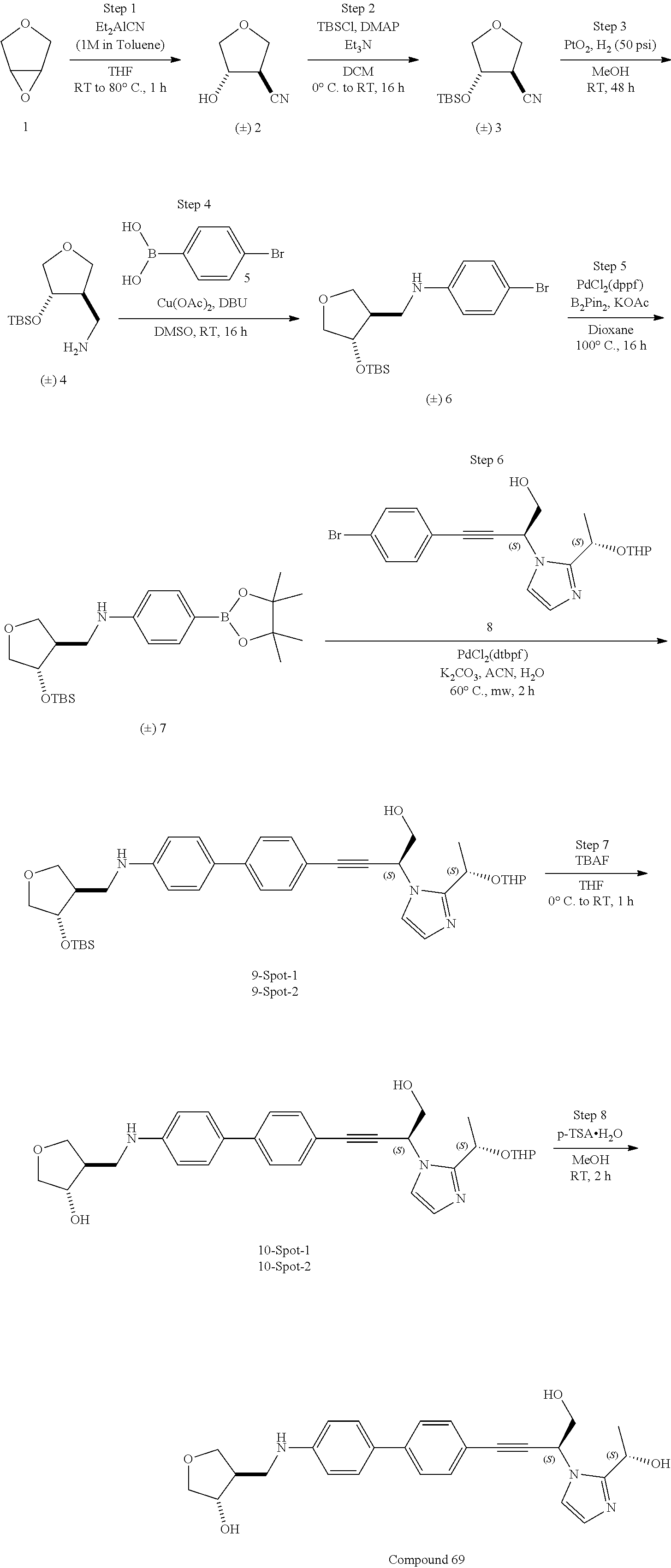

### Step 1:

To a stirred solution of 3,4-epoxytetrahydrofuran (1, 7 g, 81 mmol) in THE (120 mL) was added diethylaluminium cyanide (1M in toluene; 203 mL, 203 mmol) dropwise at room temperature. The reaction mixture was stirred at 80° C. for 1 h. The reaction mixture was quenched with Ethanol (200 mL). After 10 min, water (400 mL) was added and extracted with 10% MeOH in DCM (200 mL×2). The combined organic layer was washed with brine (100 mL), dried over anhydrous Na₂SO₄, filtered and concentrated under reduced pressure. The crude product was purified by using MPLC (manually packed cartridge, SiO₂ 230-400 mesh; 355% EtOAc in hexanes) to obtain (±)-2 as a colorless oil. Yield: 2.5 g (27%)

### Step 2:

To a stirred solution of (±)-2 (1.5 g, 13.26 mmol) in DCM (30 mL), were added DMAP (0.810 g, 6.63 mmol), triethylamine (5.53 mL, 39.8 mmol) and TBDMS-Cl (4.00 g, 26.5 mmol) at 0° C. The resulting reaction mixture was stirred at room temperature for 16 h. The reaction mixture was quenched with addition of 10% sodium bicarbonate solution (100 mL) and extracted with DCM (30 mL×2). The combined organic layer was washed with brine (30 mL), dried over anhydrous Na₂SO₄, filtered and concentrated under reduced pressure. The crude product was purified by using MPLC (manually packed cartridge, SiO₂ 230-400 mesh; 10% EtOAc in hexanes) to afford (±)-3 as a colorless oil. Yield: 2.9 g (94%)

### Step 3:

In a tiny clave to a stirred solution of (±)-3 (2.9 g, 12.75 mmol) in MeOH (30 mL), was added platinum(IV) oxide (0.145 g, 0.638 mmol) at room temperature. The reaction was stirred under hydrogen atmosphere (50 psi) for 48 h. The reaction mixture was filtered through Celite pad and the filtrate was concentrated. The crude product was purified by using MPLC (manually packed cartridge, SiO₂ 230-400 mesh; 10% MeOH in DCM) to afford (±)-4 as a brown color oil. LC-MS: Calculated for C11H25NO2Si is 231.17, Observed: 232.3 [M+1]⁺. Yield: 1.0 g (34%)

### Step 4:

To a stirred solution of (4-bromophenyl)boronic acid (5, 3.73 g, 18.58 mmol) in DMSO (20 mL), were added copper (II) acetate (0.169 g, 0.929 mmol) and DBU (2.77 mL, 18.58 mmol) at room temperature and stirred for 5 min. To this reaction mixture (±)-4 (2.15 g, 9.29 mmol) was added and stirred for 16 h under oxygen atmosphere. The reaction mixture was quenched with water (80 mL) and extracted with EtOAc (50 mL×2). The combined organic layer was washed with brine (50 mL), dried over anhydrous Na₂SO₄, filtered and concentrated under reduced pressure. The crude product was purified by using MPLC (manually packed cartridge, SiO₂ 230-400 mesh; 20% EtOAc in hexanes) to afford (±)-6 as a brown color oil in 65% purity by LCMS. The product was take as such to the next step without further purification. LC-MS: Calculated for C17H28BrNO2Si is 386.11, Observed: 386.1 [M]⁺ and 388.3 [M+2]⁺. Yield: 1.8 g (33%)

### Step 5:

To a solution of (±)-6 (1.3 g, 3.36 mmol) and bis(pinacolato)diboron (1.282 g, 5.05 mmol) in dioxane (13 mL), was added potassium acetate (0.991 g, 10.09 mmol) at room temperature. The reaction mixture was degassed for 5 min. To this reaction mixture, PdCl2(dppf) (0.246 g, 0.336 mmol) was added and heated at 100° C. for 16 h. The reaction mixture was quenched with water (80 mL) and extracted with EtOAc (100 mL×2). The combined organic layer was washed with brine (30 mL), dried over anhydrous Na₂SO₄, filtered and concentrated under reduced pressure to get the crude product. The crude product was purified by MPLC (manually packed cartridge, SiO₂ 230-400 mesh; 30% EtOAc in hexanes) to obtain (±)-7 as an off-white solid. UPLC-MS: Calculated for C23H40BNO4Si is 433.4, Observed: 434.3 [M+1]⁺. Yield: 0.425 g (29%)

### Step 6:

To a stirred solution of (±)-7 (0.380 g, 0.877 mmol) in water (4 mL) and acetonitrile (4 mL), were added 8 (0.368 g, 0.877 mmol) and potassium carbonate (0.363 g, 2.63 mmol) at room temperature. The resulting reaction mixture was purged with nitrogen for 5 min. To this reaction mixture, PdCl2(dtbpf) (0.029 g, 0.044 mmol) was added and the purging continued for another 2 min. The resulting reaction mixture was irradiated with microwave at 60° C. for 2 h. The reaction mixture was quenched with water (40 mL) and extracted with 10% MeOH in DCM (20 mL×2). The combined organic layers were washed with brine (10 mL), dried over anhydrous Na₂SO₄, filtered and concentrated under reduced pressure to afford 9-Spot-1 and 9-Spot-2 as brown color solids. LC-MS: Calculated for C37H51N3O5Si is 645.9, Observed: 646.3 [M+1]⁺. Yield: 9-Spot-1=0.16 g (27%) and 9-Spot-2=0.15 g (23%). 9-Spot-1 and 9-Spot-2 independently taken to the next steps.

### Step 7

To a stirred solution of 9-Spot-1 (0.150 g, 0.232 mmol) in THF (3.0 mL), was added TBAF (0.348 mL, 0.348 mmol) at 0° C. The resulting reaction mixture was stirred at room temperature for 1 h. The reaction mixture was quenched with sat.NH₄Cl solution (20 mL) and extracted with EtOAc (10 mL×2). The combined organic layer was washed with brine (10 mL), dried over anhydrous Na₂SO₄, filtered and concentrated under reduced pressure to get 10-Spot-1 as a brown color solid. LC-MS: Calculated for C31H37N3O5 is 531.65, Observed: 532.6 [M+1]⁺. Yield: 0.130 g (96%)

10-Spot-2 was synthesized from 9-Spot-2 (0.15 g, 0.232 mmol) by using same procedure of step-7 for 9-Spot-1. LC-MS: Calculated for C31H37N3O5 is 531.65, Observed: 532.6 [M+1]⁺. Yield: 0.12 g (93%)

### Step 8

To a solution of 10-Spot-1 (0.130 g, 0.245 mmol) in MeOH (5 mL), was added p-toluenesulfonic acid monohydrate (0.233 g, 1.223 mmol) at room temperature and the resulting reaction mixture stirred at room temperature for 2 h. The volatiles in the reaction were concentrated under reduced pressure, the crude residue was basified with sat. NaHCO3 solution (15 mL). The aqueous layer was extracted with 20% MeOH in DCM (10 mL×2). The combined organic extract was dried over anhydrous NaSO4, filtered and concentrated to afford crude mass. The crude product was purified by reverse phase preparative HPLC (Column: ZORBAX C18 (150×21.2 mm) 7 μm; Eluents: 10 mM ammonium bicarbonate in water and acetonitrile) to obtain Compound 69 as a white solid. UPLC-MS: Calculated for C26H29N3O4 is 447.5, Observed: 448.2 [M+1]⁺. Yield: 20 mg (18%). The final compound was a mixture of diastereomers; racemic at tail.

### Example A23: Synthesis of Compounds 70 and 71

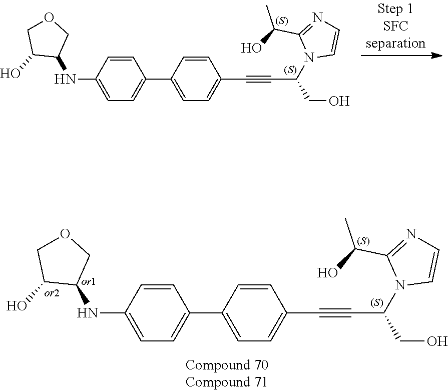

### Step-1:

The diastereomers were separated by SFC (PIC 22-027, IZ-(250*30) mm, 5 μm; eluents: CO₂ and 0.5% isopropyl amine in MeOH). The fractions were concentrated under reduced pressure to afford Compound 70 as an off-white solid and Compound 71 as a pale-yellow solid. Yield: Compound 70 (tR=13.82 min)=200 mg and Compound 71 (tR=16.49 min)=200 mg

Compound 70 (tR=13.82 min) LC-MS: Calculated for C₂₅H₂₇N₃O₄ is 433.51, Observed: 434.2 [M+1]⁺. 1H-NMR (400 MHz, DMSO-d6): δ 7.60 (d, J=8.4, 2H), 7.49-7.45 (m, 4H), 7.35 (d, J=1.2 Hz, 1H), 6.84 (d, J=1.2 Hz, 1H), 6.74 (d, J=8.8 Hz, 2H), 6.07 (d, J=6.0 Hz, 1H, exchanges with D2O), 5.68 (t, J=6.0 Hz, 1H), 5.52 (t, J=5.6 Hz, 1H, exchanges with D2O), 5.36 (d, J=5.6 Hz, 1H, exchanges with D2O), 5.29 (d, J=3.6 Hz, 1H, exchanges with D2O), 4.96-4.91 (m, 1H), 4.09-4.05 (m, 2H), 3.88-3.84 (m, 3H), 3.69 (t, J=8.8 Hz, 1H), 3.63 (dd, J=2.4, 8.8 Hz, 1H), 3.57 (dd, J=1.2, 9.4 Hz, 1H), 1.51 (d, J=6.8 Hz, 3H).

SFC: 100%; tR=13.82 min (Column: LUX C2; eluents: CO2 and 0.5% isopropyl amine in MeOH). Single isomer with SFC purity 100%

Compound 71 (tR=16.49 min). LC-MS: Calculated for C₂₅H₂₇N₃O₄ is 433.51, Observed: 434.2 [M+1]⁺. 1H-NMR (400 MHz, DMSO-d6): δ 7.60 (d, J=8.4 Hz, 2H), 7.49-7.45 (m, 4H), 7.35 (d, J=1.2 Hz, 1H), 6.84 (d, J=1.2 Hz, 1H), 6.74 (d, J=8.8 Hz, 2H), 6.07 (d, J=6.0 Hz, 1H), 5.68 (t, J=6.0 Hz, 1H), 5.53 (t, J=5.6 Hz, 1H, exchanges with D2O), 5.37 (d, J=5.6 Hz, 1H, exchanges with D2O), 5.29 (d, J=3.6 Hz, 1H, exchanges with D2O), 4.96-4.92 (m, 1H), 4.09-4.05 (m, 2H), 3.88-3.84 (m, 3H), 3.69 (t, J=8.8 Hz, 1H), 3.63 (dd, J=2.0, 8.8 Hz, 1H), 3.57 (dd, J=1.2, 9.2 Hz, 1H), 1.51 (d, J=6.40 Hz, 3H). SFC: 97.5%; tR=16.49 min (Column: LUX C2; eluents: CO2 and 0.5% isopropyl amine in MeOH). diastereomeric ratio=97.5:2.5

### Example A24: Synthesis of Compounds 72 and 73

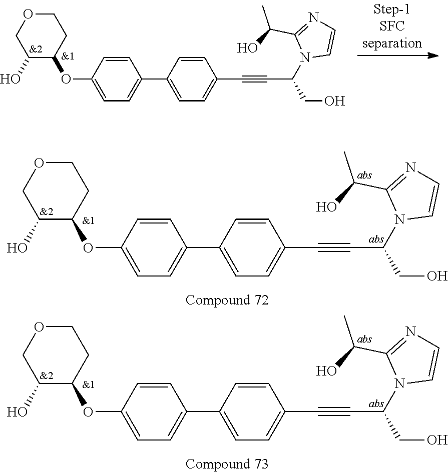

### Step 1:

600 mg of the starting material was separated by using SFC (Column: I Amylose A (250×30) mm, 5 μm; Eluents: CO2:0.5% IPAm in Methanol [65:35]) to get Compound 72 (t_{R}=4.0 min) as an off-white solid and Compound 73 (t_{R}=7.85 min) as an off -white solid. Yield: Compound 72=160 mg and Compound 73=200 mg. UPLC-MS: Calculated for C26H28N2O5 is 448.5, Observed: 449.0 [M+1]⁺. Diastereomeric excess (de) Compound 72=100% and Compound 73=98.5%

### Example A25: Synthesis of Compound 74

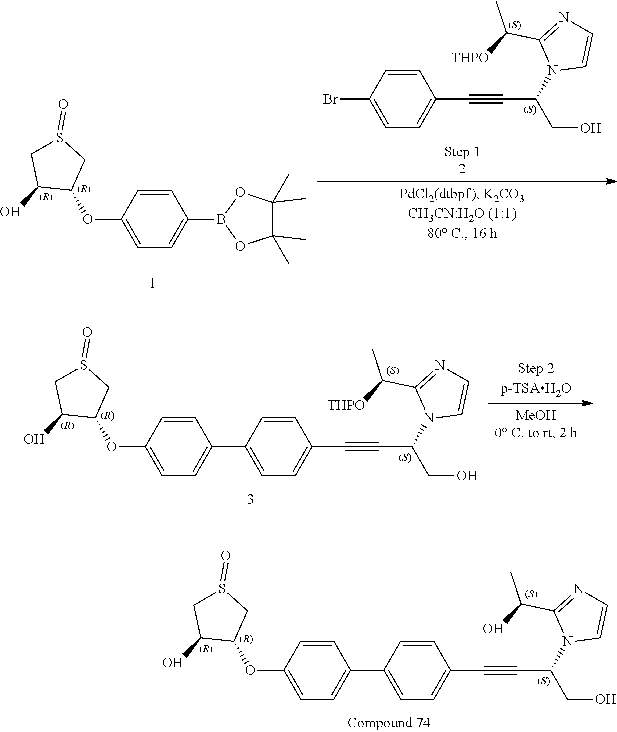

### Step-1:

To a stirred solution of 1 (1.2 g, 2.86 mmol) in ACN (25 mL) and H2O (25 mL), were added 2 (1.886 g, 4.29 mmol) and K2CO3 (1.187 g, 8.59 mmol) at room temperature. The reaction mixture was purged with nitrogen for 5 min. To this reaction mixture, PdCl2(dtbpf) (0.186 g, 0.286 mmol) was added. The reaction mixture was heated to 80° C. and stirred for 16 h. The reaction mixture was diluted with 50 mL of water and extracted with EtOAc (3×50 mL). The combined organic extract was washed with brine (50 mL), dried over anhydrous sodium sulphate, filtered, and concentrated to afford crude residue. The crude residue was purified by MPLC (manually packed cartridge, SiO₂ 230-400 mesh size; 5% MeOH in DCM) to afford 3 as a brown solid. Yield: 550 mg (33%). LC-MS: Calculated for C30H34N2O6S is 550.21, Observed: 551.0 [M+H]⁺

### Step-2:

To a stirred solution of 3 (500 mg, 0.908 mmol) in MeOH (20 mL), was added p-toluene sulfonic acid monohydrate (518 mg, 2.72 mmol) at 0° C. The reaction mixture was stirred at room temperature for 2 h. The reaction was quenched with sat. NaHCO3 solution (20 mL) at 0° C. and extracted with 10% MeOH in DCM (100 mL×2). The combined organic extract was washed with brine (30 mL), dried over anhydrous sodium sulphate, filtered, and concentrated to afford crude residue. The crude residue was purified by MPLC (manually packed cartridge, SiO₂ 230-400 mesh size; 5% MeOH in DCM) to afford Compound 74 as a brown solid. Yield: 165 mg (38%). LC-MS: Calculated for C25H26N2O5S is 466.16, Observed: 467.0 [M+H]⁺. SFC and NMR showed single isomer; unknown stereochemistry at sulfoxide.

### Example A26: Synthesis of Compound 75

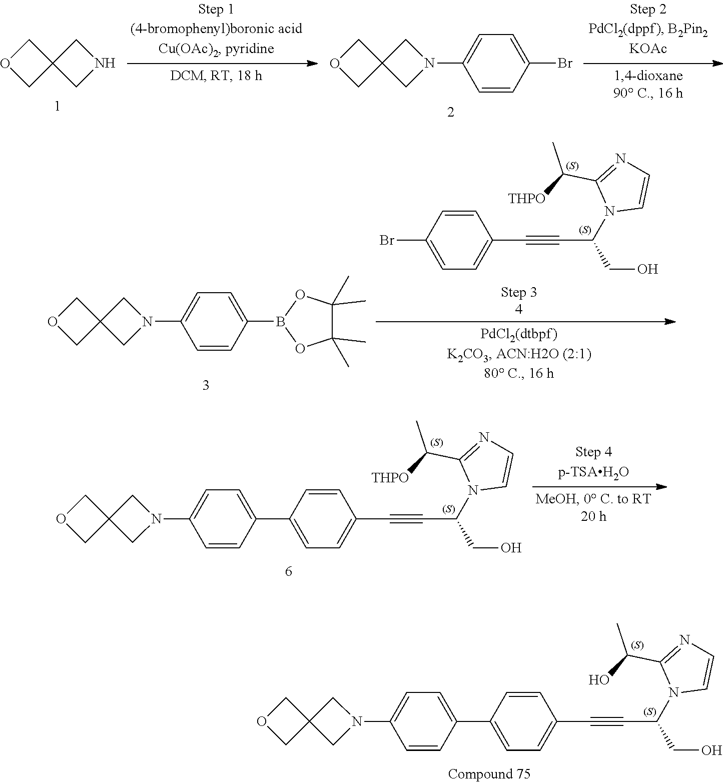

### Step 1:

To a stirred solution of 2-oxa-6-azaspiro[3.3]heptane (1, 1.0 g, 10.09 mmol) and (4-bromophenyl)boronic acid (4.05 g, 20.17 mmol) in DCM (20 mL), were added pyridine (2.448 mL, 30.3 mmol) and copper(II) acetate (5.50 g, 30.3 mmol) at room temperature and the resulting reaction mixture stirred at room temperature for 18 h under oxygen atmosphere. The reaction mixture was filtered through Celite bed. The Celite bed was washed with DCM (100 mL), the filtrate, combined and evaporated under reduced pressure. The crude residue, thus obtained, was purified by using MPLC (manually packed SiO₂ cartridge; 230-400 mesh size; 10% EtOAc in hexanes) to obtain 2 as an off-white solid. Yield: 1.0 g (38%). LCMS: Calculated for C₁₁H₁₂BrNO is 254.13, Observed: 254.0 [M]⁺ and 256.0 [M+2]⁺

### Step 2:

To a stirred solution of 2 (1.0 g, 3.94 mmol) in 1,4-dioxane (20 mL), were added potassium acetate (0.772 g, 7.87 mmol) and bis(pinacolato)diboron (1.499 g, 5.90 mmol) at room temperature and the reaction mixture purged using nitrogen for 5 min. To this reaction mixture, PdCl₂(dppf) (0.288 g, 0.394 mmol) was added, the purging continued for 5 min, and the reaction mixture then stirred at 90° C. for 16 h. The reaction mixture was cooled to room temperature and quenched with water (10 mL) and extracted with EtOAc (2×20 mL). The combined organic extract was washed with brine solution (15 mL), dried over anhydrous sodium sulphate, filtered, and concentrated under reduced pressure. The crude residue, thus obtained, was purified by using MPLC (manually packed SiO₂ cartridge; 230-400 mesh size; 12% EtOAc in hexanes) to get 3 as white solid. Yield: 680 mg (55%). LCMS: Calculated for C₁₇H₂₄BNO₃ is 301.19, Observed: 302.3 [M+1]⁺

### Step 3:

To the stirred solution of 4 (500 mg, 1.192 mmol) in acetonitrile (6 mL) and water (3 mL), were added 3 (431 mg, 1.431 mmol) and K₂CO₃ (494 mg, 3.58 mmol) at room temperature, and the resulting reaction mixture purged with nitrogen for 5 min. To this reaction mixture, PdCl₂(dtbpf) (38.9 mg, 0.060 mmol) was added, and the purging was continued for 5 min. The resulting reaction mixture was heated to 80° C. for 16 h. The reaction mixture was cooled to room temperature, quenched with ice-cold water (15 mL) and extracted with 5% MeOH in DCM (3×20 mL). The combined organic extract was washed with brine solution (10 mL), dried over anhydrous sodium sulphate, filtered, and concentrated under reduced pressure. The crude residue, thus obtained, was purified by using MPLC (manually packed SiO₂ cartridge; 230-400 mesh size; 4% MeOH in DCM) to afford 5 as brown solid. Yield: 310 mg (48%). LCMS: Calculated for C₃₁H₃₅N₃O₄ is 513.64., Observed: 514.3 [M+1]⁺

### Step 4:

To the stirred solution 5 (300 mg, 0.584 mmol) in MeOH (40 mL), was added p-TSA·H₂O (222 mg, 1.168 mmol) at 0° C. and the resulting reaction mixture and stirred for 20 h at room temperature. The reaction mixture was quenched with sat. NaHCO₃ solution (8 mL) at 0° C. and extracted with DCM (3×50 mL). The combined organic extract was washed with sat. NaHCO₃ solution (2×8 mL), followed by brine solution (15 mL), dried over anhydrous sodium sulphate, filtered, and concentrated. The crude residue, thus obtained, was purified by using MPLC (manually packed SiO₂ cartridge, 230-400 mesh size; 6% MeOH in DCM) to afford Compound 75 as an off-white solid. Yield: 65 mg (25%). LCMS: Calculated for C₂₆H₂₇N₃O₃ is 429.21, Observed: 430.2 [M+1]⁺. 1H-NMR (400 MHz, DMSO-d₆): δ 7.60 (d, J=8.4 Hz, 2H), 7.54 (d, J=8.8 Hz, 2H), 7.47 (d, J=8.4 Hz, 2H), 7.35 (app d, J=0.8 Hz, 1H), 6.84 (app d, J=1.2 Hz, 1H), 6.52 (d, J=8.8 Hz, 2H), 5.68 (t, J=5.6 Hz, 1H), 5.52 (t, J=5.6 Hz, 1H, exchanges with D2O), 5.36 (d, J=5.6 Hz, 1H, exchanges with D2O), 4.97-4.91 (m, 1H), 4.73 (s, 4H), 4.02 (s, 4H), 3.86 (t, J=6.0 Hz, 2H), 1.51 (d, J=6.4 Hz, 3H). SFC: 95.56%; tR=1.46 min (Column: CHIRALPAK AS-H; Eluents: 0.5% isopropyl amine in MeOH and CO2). Single isomer with 95.5% SFC purity.

### Example A27: Synthesis of Compound 76

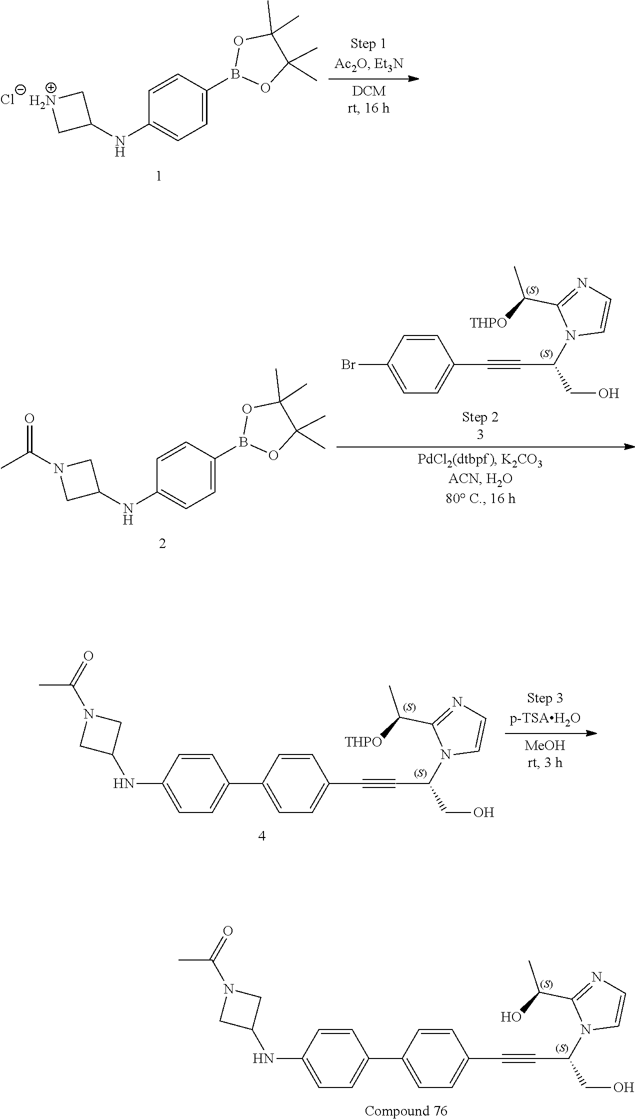

### Step 1:

To a stirred solution of 1 (1.2 g, 3.86 mmol) in DCM (20 mL), were added triethylamine (0.661 mL, 4.64 mmol) and acetic anhydride (0.359 mL, 3.86 mmol) at 0° C., and the reaction mixture stirred at room temperature under nitrogen for 16 h. The reaction mixture was quenched with water (20 mL) and extracted with DCM (2×15 mL). The combined organic layer was dried over anhydrous sodium sulfate, filtered, and concentrated under reduced pressure. The crude residue, thus obtained, was purified by MPLC (manually packed SiO₂ cartridge, 100-200 mesh size; Eluent: 10% MeOH in DCM to obtain 2 as an off-white solid with 510% purity by LC-MS; the product was taken to the next step without further purification. Yield: 700 mg (29%). LC-MS: Calculated for C₁₇H₂₅BN₂O₃ is 316.21, Observed: 317.2 [M+1]⁺

### Step 2:

To a stirred solution of 3 (500 mg, 1.192 mmol) in acetonitrile (20 mL) and water (2 mL), were added 2 (566 mg, 1.789 mmol) and potassium carbonate (494 mg, 3.58 mmol) at room temperature, and a stream of nitrogen passed through the reaction mixture for 5 min. To this reaction mixture, PdCl₂(dtbpf) (38.8 mg, 0.060 mmol) was added and the bubbling of nitrogen continued for 5 min. The reaction mixture was then stirred at 80° C. for 16 h. The reaction mixture was cooled to room temperature, quenched with water (20 mL), extracted with EtOAc (2×20 mL). The combined organic layer was washed with brine (10 mL), dried over anhydrous sodium sulfate, filtered, and concentrated under reduced pressure. The crude residue, thus obtained, was purified by MPLC (manually packed SiO₂ cartridge, 100-200 mesh size; Eluents: 10% MeOH in DCM) to obtain 4 as a brown gum. Yield: 170 mg (23%). LC-MS: Calculated for C₃₁H₃₆N₄O₄ is 528.6, Observed: 529.3 [M+1]⁺

### Step 3:

To a stirred solution of 4 (150 mg, 0.284 mmol) in MeOH (5 mL), was added p-toluene sulfonic acid monohydrate (162 mg, 0.851 mmol) at 0° C. and the resulting reaction mixture stirred at room temperature for 3 h. The volatiles were evaporated under reduced pressure. The resulting crude residue was basified with sat. NaHCO₃ solution (10 mL). The aqueous layer was extracted with 10% MeOH in DCM (2×10 mL). The combined organic extract was dried over anhydrous sodium sulfate, filtered, and concentrated under reduced pressure. The crude residue thus obtained was purified by reversed-phase preparative HPLC (Column: Shimpack C₁₈ (150*20 mm) 5 μm, Eluents: 10 mM ammonium bicarbonate in water and acetonitrile) to obtain 40 mg of product. 1H NMR showed extraneous peaks with SFC purity 92.5%. The product was re-purified by SFC (Column: I Cellulose-J; Eluents: CO₂:0.5% isopropyl amine in MeOH) to obtain Compound 76 as an off-white solid. Yield: 18 mg (14%). LC-MS: Calculated for C₂₆H₂₈N₄O₃ is 444.54, Observed: 445.2 [M+1]⁺. 1H-NMR (400 MHz, DMSO-d6): δ 7.60 (d, J=8.40 Hz, 2H), 7.50 (d, J=8.80 Hz, 2H), 7.47 (d, J=8.40 Hz, 2H), 7.35 (app d, J=1.20 Hz, 1H), 6.84 (app d, J=0.80 Hz, 1H), 6.61 (d, J=8.80 Hz, 2H), 6.55 (d, J=6.00 Hz, 1H, exchanges with D2O), 5.68 (t, J=6.00 Hz, 1H), 5.52 (t, J=5.60 Hz, 1H, exchanges with D2O), 5.36 (d, J=5.60 Hz, 1H, exchanges with D2O), 4.94-4.92 (m, 1H), 4.49-4.45 (m, 1H), 4.24-4.17 (m, 2H), 3.88-3.85 (m, 3H), 3.66-3.64 (m, 1H), 1.78 (s, 3H), 1.51 (d, J=6.40 Hz, 3H). SFC: 100%; tR=5.25 min (Column: I Cellulose-J; Eluents: CO2 and 0.5% isopropyl amine in MeOH). Single isomer with 100% SFC purity.

### Example A28: Synthesis of Compounds 77 and 78

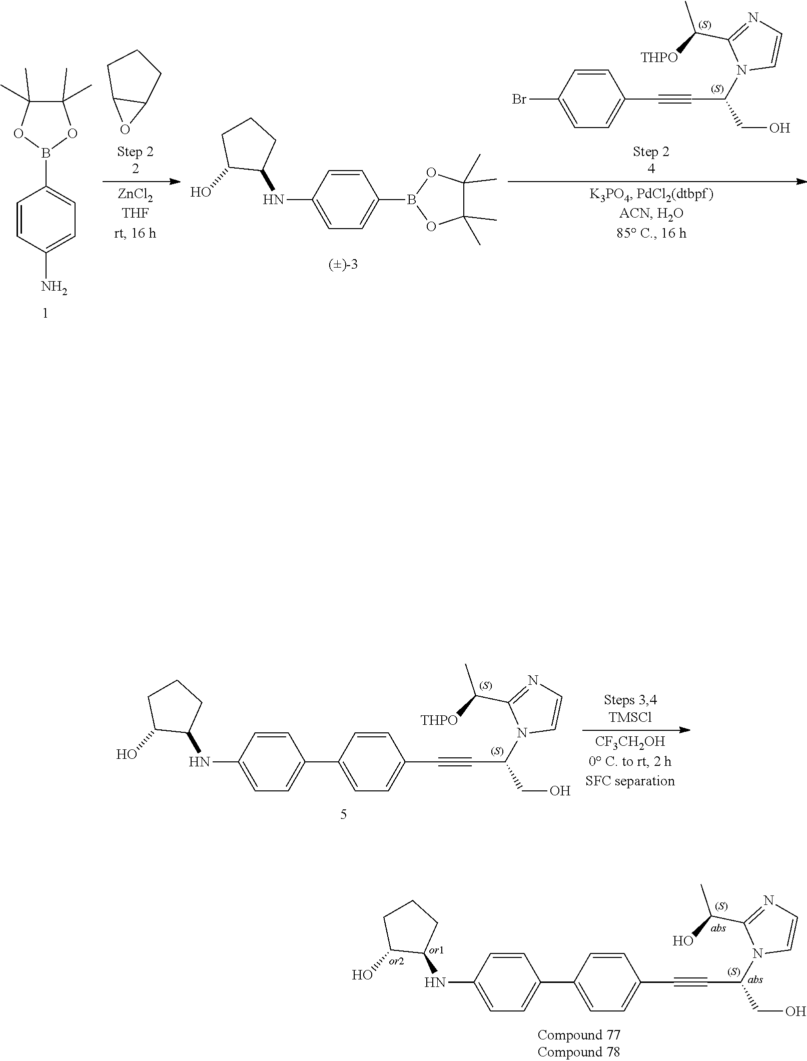

### Step-1:

To a stirred solution of 4-(4,4,5,5-tetramethyl-1,3,2-dioxaborolan-2-yl)aniline (1, 3.0 g, 13.69 mmol) in THF (30 mL), were added 3,4-epoxytetrahydrofuran (2, 1.152 g, 13.69 mmol) and zinc chloride (0.187 g, 1.369 mmol) at room temperature. The reaction mixture was stirred at room temperature for 16 h. The reaction was quenched with saturated NaHCO₃ solution (30 mL) and extracted with EtOAc (3×150 mL). The combined organic layer was washed with brine (150 mL), dried over anhydrous Na₂SO₄, filtered and concentrated under reduced pressure. The resulting crude mass, thus obtained, was purified by MPLC (using manually packed SiO2 cartridge, 100-200 mesh; 25% EtOAc in hexanes) to afford (±)-3 as a viscous brown liquid. Yield: 1.1 g (25%). LC-MS: Calculated for C₁₇H₂₆BNO₃ is 303.20, Observed: 304.3 [M+H]⁺

### Step-2:

To a stirred solution of 4 (0.5 g, 1.192 mmol) in dioxane (5 mL) and water (2 mL), were added 3 (0.542 g, 1.789 mmol) and potassium phosphate tribasic (0.759 g, 3.58 mmol) at room temperature. The reaction mixture was purged with nitrogen for 5 min. To this reaction mixture, PdCl2(dtbpf) (0.039 g, 0.060 mmol) was added and was purging continued for another 2 min. The resulting reaction mixture was stirred at 85° C. for 16 h. The reaction was cooled to ambient temperature and filtered through a Celite bed. The Celite bed was washed with 10% MeOH in DCM (2×100 mL). The combined filtrate was washed with brine (50 mL), dried over anhydrous sodium sulphate, filtered and concentrated under reduced pressure. The resulting crude reside was purified by MPLC (using manually packed SiO₂ cartridge, 230-400 mesh size; 7% MeOH in EtOAc) to afford 5 a brown solid. Yield: 0.5 g (81%). LC-MS: Calculated for C₃₁H₃₇N₃O₄ is 515.65, Observed: 516.2 [M+H]⁺. One more batch was carried out on 500 mg of 4 to get 400 mg of 5.

### Step-3:

To a stirred solution of 5 (0.5 g, 0.970 mmol) in 2,2,2-trifluoroethanol (5 mL), was added TMSCl (1.0 M in THF) (0.970 mL, 0.970 mmol) at 0° C. The reaction mixture was stirred at room temperature for 2 h. The reaction was quenched with saturated NaHCO₃ solution (15 mL) and extracted with EtOAc (2×150 mL). The combined organic layer was washed with brine (50 mL), dried over anhydrous Na₂SO₄, filtered and concentrated reduced pressure to get crude residue. The resulting crude mass was purified by MPLC (using manually packed SiO₂ cartridge, 230-400 mesh size; 90-100% EtOAc in hexanes) to afford the product as a brown solid. Yield: 84 mg (20%). LC-MS: Calculated for C₂₆H₂₉N₃O₃ is 431.53, Observed: 432.3 [M+1]⁺. One more batch was carried out on 200 mg of 5 to get 76 mg of the product.

### Step-4:

The diastereomers (0.16 g) were separated by SFC (PIC 22-016, IB-(250*21) mm, 5 μm; eluents: CO2:0.5% isopropyl amine in MeOH [65:35]). The fractions were concentrated under reduced pressure to afford Compound 77 ((tR=7.17 min) and Compound 78 (tR=9.31 min).

Compound 77: LC-MS: Calculated for C₂₆H₂₉N₃O₃ is 431.53, Observed: 432.3 [M+1]⁺. 1H-NMR (400 MHz, DMSO-d6): δ 7.58 (dd, J=2.00, 6.80 Hz, 2H), 7.45 (dd, J=2.40, 8.60 Hz, 4H), 7.35 (app d, J=1.20 Hz, 1H), 6.84 (app d, J=1.20 Hz, 1H), 6.70 (d, J=8.80 Hz, 2H), 5.80 (d, J=6.00 Hz, 1H, exchanges with D2O), 5.68 (t, J=6.00 Hz, 1H), 5.52 (t, J=5.60 Hz, 1H, exchanges with D2O), 5.36 (d, J=5.60 Hz, 1H, exchanges with D2O), 4.95-4.92 (m, 1H), 4.74 (d, J=3.60 Hz, 1H, exchanges with D2O), 3.90-3.85 (m, 3H), 3.46-3.45 (m, 1H), 2.12-2.10 (m, 1H), 1.85-1.66 (m, 3H), 1.53-1.51 (m, 3H), 1.50-1.24 (m, 2H).

Compound 78: LC-MS: Calculated for C₂₆H₂₉N₃O₃ is 431.53, Observed: 432.3 [M+1]⁺. 1H-NMR (400 MHz, DMSO-d6): δ 7.58 (dd, J=2.00, 6.60 Hz, 2H), 7.45 (dd, J=2.00, 8.80 Hz, 4H), 7.35 (app d, J=1.60 Hz, 1H), 6.84 (app d, J=1.20 Hz, 1H), 6.70 (d, J=8.80 Hz, 2H), 5.80 (d, J=6.00 Hz, 1H, exchanges with D2O), 5.68 (t, J=6.00 Hz, 1H), 5.52 (t, J=5.60 Hz, 1H, exchanges with D2O), 5.36 (d, J=5.60 Hz, 1H, exchanges with D2O), 4.95-4.92 (m, 1H), 4.74 (d, J=4.00 Hz, 1H, exchanges with D2O), 3.90-3.85 (m, 3H), 3.46-3.45 (m, 1H), 2.12-2.08 (m, 1H), 1.81-1.77 (m, 1H), 1.72-1.66 (m, 2H), 1.53-1.51 (m, 3H), 1.50-1.42 (m, 2H). Yield: Compound 77 (tR=7.17 min)=38 mg and Compound 78 (tR=9.31 min)=32 mg.

For Compound 77 (tR=7.17 min): Single isomer with SFC purity 99.5% (l Cellulose-B_0.5% IPAm in MeOH tR=7.17 min) and for Compound 78 (tR=9.31 min) from SFC (l-Cellulose-B_0.5% IPAm in MeOH tR=9.31 min), dr=99.4:0.15

### Example A29: Synthesis of Compound 79

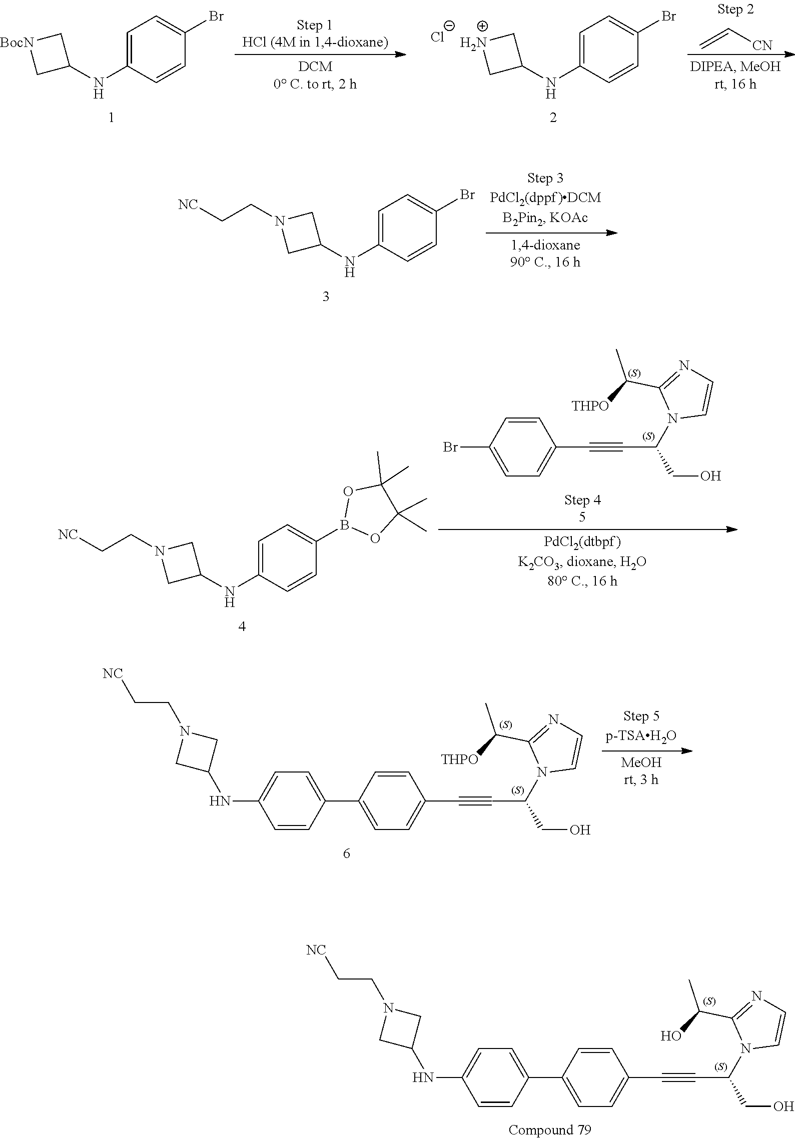

### Step 1:

To a stirred solution of tert-butyl 3-((4-bromophenyl)amino)azetidine-1-carboxylate (1, 6.0 g, 18.34 mmol) in DCM (50 mL), was added HCl (4 M in 1,4 dioxane, 13.75 mL, 55.0 mmol) at 0° C., and the reaction mixture stirred at room temperature for 2 h. The reaction mixture was concentrated under reduced pressure. The resulting residue was co-distilled with toluene (20 mL) and dried under reduced pressure to afford 2 as an off-white solid. The product was taken to the next step without any purification. Crude yield: 5 g. LCMS: Calculated for C₉H₁₁BrN₂⁺ is 227.02 (exact mass); Observed: 227.2 [M]⁺ and 229.2 [M+2]⁺

### Step 2:

To a stirred solution of 2 (3.5 g, 13.28 mmol) in MeOH (35 mL), was added DIPEA (6.96 mL, 39.8 mmol) at 0° C. After 10 min, acrylonitrile (1.305 mL, 19.92 mmol) was added and the reaction mixture stirred at 25° C. for 16 h. The reaction mixture was cooled to 0° C., quenched with ice cold water (5 mL) and concentrated under reduced pressure. The resulting crude residue was dissolved in 10% MeOH in DCM (25 mL), washed with water (10 mL), brine solution (5 mL), dried over anhydrous Na₂SO₄, filtered and concentrated under reduced pressure. The crude residue, thus obtained, was purified by using MPLC (manually packed SiO₂ cartridge, 230-400 mesh, 80% EtOAc in hexanes) to afford 3 as a pale-yellow gum. Yield: 1.6 g (40%). LCMS: Calculated C₁₂H₁₄BrN₃ is 280.17, Observed: 280.2 [M]⁺ and 282.2 [M+2]⁺

### Step 3:

To a stirred solution of 3 (1.6 g, 5.71 mmol) in 1,4-dioxane (20 mL), were added bis(pinacolato)diboron (2.32 g, 9.14 mmol) and potassium acetate (1.681 g, 17.13 mmol) at room temperature, and a stream of nitrogen gas bubbled through the mixture for 15 min. PdCl2(dppf)·CH₂Cl₂ adduct (0.466 g, 0.571 mmol) was added, and the reaction mixture heated to 90° C. for 16 h. The reaction mixture was cooled to 25° C., diluted with EtOAc (30 mL), filtered through the Celite pad. The pad was washed with EtOAc (2×30 mL), the filtrate combined and concentrated under reduced pressure. The crude residue, thus obtained, was purified by using MPLC (manually packed SiO₂ cartridge, 230-400 mesh; 80% EtOAc in hexanes) to afford 4 as a pale-brown gum. Yield: 1.16 g (52%). LCMS: Calculated C₁₈H₂₆BN₃O₂ is 327.23; Observed: 328.3 [M+1]⁺

### Step 4:

To a stirred solution of 4 (375 mg, 1.145 mmol) in 1,4-dioxane (10 mL) and water (3 mL), were added 5 (400 mg, 0.954 mmol) and K2CO3 (396 mg, 2.86 mmol) at 25° C., and a stream of nitrogen gas bubbled through the mixture for 10 min. To this reaction mixture, PdCl2(dtpbf) (31.1 mg, 0.048 mmol) was added at 25° C. and the reaction mixture heated at 80° C. for 16 h. The reaction mixture was cooled to 25° C., quenched with water (5 mL). This was extracted with 10% MeOH in DCM (2×10 mL). The combined organic layer was washed with water (3 mL), brine (3 mL), dried over Na₂SO₄, filtered, and concentrated under reduced pressure. The crude residue thus obtained was purified by using MPLC (manually packed SiO₂ cartridge, 230-400 mesh, 9% MeOH in EtOAc) to afford 6 as a pale-brown semi solid. Yield: 330 mg (59%). LCMS: Calculated C₃₂H₃₇N₂O₃ is 539.68; Observed: 540.3 [M+1]⁺

### Step 5:

To a stirred solution of 6 (330 mg, 0.611 mmol) in MeOH (30 mL), was added p-toluenesulfonic acid monohydrate (353 mg, 1.834 mmol) at 0° C. and the reaction mixture stirred at 25° C. for 3 h. The reaction mixture was diluted with DCM (150 mL) and quenched with ice cold H₂O (5 mL). The organic layer was separated, washed with saturated NaHCO₃ solution (2×5 mL), brine solution (5 mL), dried over anhydrous Na₂SO₄, filtered and concentrated under reduced pressure. The crude residue, thus obtained, was purified by using preparative HPLC (column: SHIMPACK GIST C18 (10×150 nm) 5 μm; eluents: 10 mM ammonium bicarbonate in water and ACN) to afford Compound 79 as an off-white solid. Yield: 35 mg (12%). LCMS: Calculated C₂₇H₂₉N₅O₂ is 455.23, observed: 456.2 [M+1]⁺. 1H-NMR (400 MHz, DMSO-d6): δ 7.58 (d, J=8.40 Hz, 2H), 7.59-7.44 (m, 4H), 7.35 (app d, J 1.20 Hz, 1H), 6.84 (app d, J=0.80 Hz, 1H), 6.60 (d, J=8.80 Hz, 2H), 6.33 (d, J=6.80 Hz, 1H, exchanges with D2O), 5.68 (t, J=6.00 Hz, 1H), 5.52 (t, 1H, exchanges with D2O), 5.36 (d, J=5.20 Hz, 1H, exchanges with D2O), 4.94 (t, J=5.60 Hz, 1H), 4.04-4.02 (m, 1H), 3.86 (t, J=5.20 Hz, 2H), 3.71 (t, J=7.20 Hz, 2H), 2.88 (t, J=7.20 Hz, 2H), 2.66-2.62 (m, 2H), 2.54-2.51 (m, 2H, exchanges with D2O, merges with solvent peak), 1.50 (d, J=6.40 Hz, 3H). SFC: 100%; tR=3.59 min (Column: Whelk-(R,R); Eluents: CO2 and 0.5% isopropyl amine in MeOH). Single isomer with SFC purity 100%

### Example A30: Synthesis of Compound 80

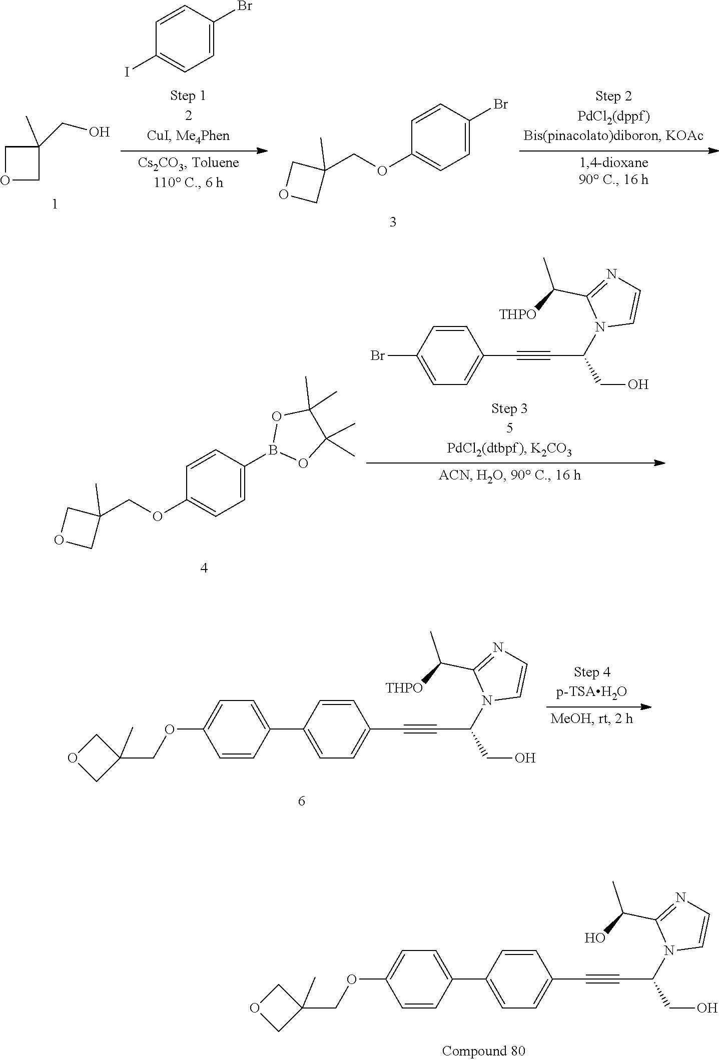

### Step 1:

To a stirred solution of Copper(I) iodide (0.286 g, 1.502 mmol) and 3,4,7,8-tetramethyl-1,10-phenanthroline (Me4Phen, 0.710 g, 3.00 mmol) in toluene (50 mL) was added cesium carbonate (19.58 g, 60.1 mmol) at room temperature. The resulting reaction mixture was purged with N₂ for 5 min. Then (3-methyloxetan-3-yl)methanol (1, 3.07 g, 30.0 mmol) and 1-bromo-4-iodobenzene (2, 8.5 g, 30.0 mmol) were added and the reaction mixture heated at 110° C. for 16 h. The reaction mixture was cooled to room temperature and quenched with water (80 mL). This was extracted with EtOAc (3×50 mL). The combined organic layer was dried over anhydrous Na2SO4, filtered and the filtrate was concentrated under reduced pressure to get a crude residue, which was purified by MPLC (using a manually packed SiO₂ cartridge, 230-400 mesh size; 8% EtOAc in hexanes) to get 3 as a brown solid. Yield: 4.97 g (57%). LCMS: Calculated for C11H13BrO2 is 257.1, Observed: Desired mass not observed.

### Step 2:

To a stirred solution of 3 (5.0 g, 19.45 mmol) and bis(pinacolato)diboron (7.38 g, 29.2 mmol) in 1,4-dioxane (80 mL), was added potassium acetate (5.72 g, 58.3 mmol) at room temperature, and the resulting mixture purged with nitrogen for 5 min. Then, Pd(dppf)Cl2 (1.421 g, 1.945 mmol) was added under nitrogen. The reaction mixture was heated at 90° C. for 16 h. The reaction mixture was cooled to room temperature and quenched with water (100 mL). This was extracted with EtOAc (3×50 mL). The combined organic layer was dried over anhydrous Na2SO4, filtered and the filtrate was concentrated under reduced pressure to get a crude residue which was purified by MPLC (using a manually packed SiO₂ cartridge, 60-120 mesh size; 8% EtOAc in hexanes) to get 4 as a light-yellow liquid. Yield: 1.8 g (12%)

### Step 3:

To a stirred solution of 5 (0.5 g, 1.192 mmol) and 4 (0.725 g, 2.385 mmol) in a mixture of acetonitrile (10 mL) and water (2 mL), was added K₂CO₃ (0.412 g, 2.98 mmol) at room temperature. The resulting mixture was purged with nitrogen for 5 min. Then Pd(dtbpf)Cl₂ (0.014 g, 0.119 mmol) was added and the reaction mixture heated at 90° C. for 18 h. The reaction mixture was cooled to room temperature and quenched with water (30 mL). This was extracted with EtOAc (2×40 mL). The combined organic layer was washed with brine (30 mL). The organic layer was separated, dried over anhydrous Na2SO4, filtered and the filtrate concentrated under reduced pressure. The crude residue, thus obtained, was purified by MPLC (using a manually packed cartridge, SiO₂230-400 mesh size; 5% MeOH in DCM) to get 6 as a light-brown solid. LCMS showed 36% purity; the product was taken as such to the next step without further purification. Yield: 0.5 g (29%). LCMS: Calculated for C₃₁H₃₆N₂O₅ is 516.6, Observed: 517.0 [M+1]⁺

### Step 4:

To a stirred solution of 6 (0.45 g, 0.314 mmol) in MeOH (15 mL), was added p-toluenesulfonic acid monohydrate (0.179 g, 0.941 mmol) at room temperature and the reaction mixture stirred at room temperature for 2 h. The reaction mixture was then quenched with aqueous 10% NaHCO₃ solution (10 mL). This was extracted with DCM (2×20 mL). The combined organic layer was dried over anhydrous Na2SO4, filtered and concentrated under reduced pressure to get a crude residue, which was purified by reversed-phase preparative HPLC (Column: ZORBAC C18 (50*21.2 mm) 5 μm, C18; Eluents: 0.1% ammonium bicarbonate in water and ACN) to obtain Compound 80 as a white solid. Yield: 18.5 mg (13%). LCMS: Calculated for C26H28N2O4 is 432.5, Observed: 433.2 [M+1]⁺. 1H-NMR (400 MHz, DMSO-d6): δ 7.68-7.65 (m, 4H), 7.52 (d, J=8.40 Hz, 2H), 7.36 (d, J=1.20 Hz, 1H), 7.09 (d, J=8.80 Hz, 2H), 6.84 (d, J=0.80 Hz, 1H), 5.68 (t, 1H), 5.53 (t, J=5.20 Hz, 1H, exchanges with D2O), 5.37 (d, J=5.60 Hz, 1H, exchanges with D2O), 4.97-4.91 (m, 1H), 4.51 (d, J=5.60 Hz, 2H), 4.32 (d, J=6.00 Hz, 2H), 4.11 (s, 2H), 3.87 (t, J=5.20 Hz, 2H), 1.51 (d, J=6.40 Hz, 3H), 1.38 (s, 3H). Single isomer with SFC purity=99.4% (Whelk-(R,R)_0.5% IPAm in MeOH tR=2.71 min).

### Example A31: Synthesis of Compound 81

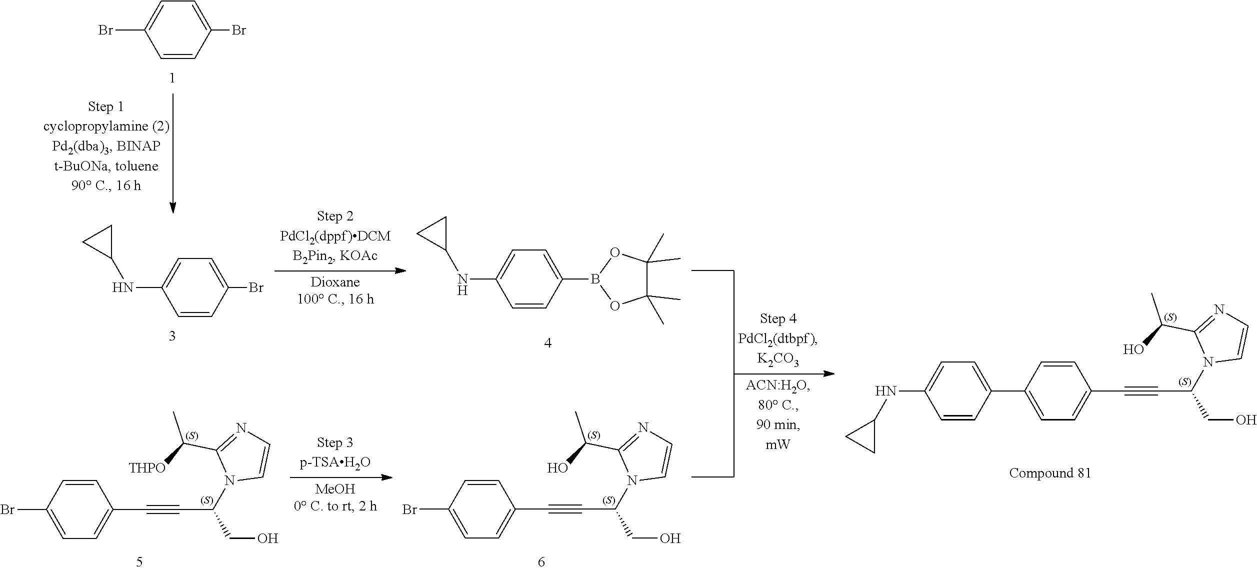

### Step-1:

A stream of nitrogen gas was bubbled through a solution of BINAP (0.264 g, 0.424 mmol) in toluene (30 mL) for 5 min following which sodium tert-butoxide (1.528 g, 15.90 mmol), cyclopropylamine (2, 1.836 mL, 26.5 mmol) and 1,4-dibromobenzene (1, 2.5 g, 10.60 mmol) and Pd2(dba)3 (0.194 g, 0.212 mmol) were added, and then heated at 90° C. for 16 h. The reaction was filtered through the Celite pad and washed with EtOAc (100 mL). The combined filtrate was concentrated under reduced pressure. The resulting crude residue was purified by using MPLC (manually packed SiO₂ cartridge, 100-200 mesh size; 2% EtOAc in hexanes) to obtain 3 as colorless gum. Yield: 0.6 g (21%). LCMS: Calculated for C₉H₁₀BrN is 212.09, Observed: 212.0 [M]⁺ and 214.2 [M+2]⁺

### Step 2:

To a stirred solution of 3 (600 mg, 2.83 mmol) in dioxane (20 mL), were added potassium acetate (694 mg, 7.07 mmol) and bis(pinacolato)diboron (862 mg, 3.39 mmol) at room temperature and a stream of nitrogen gas was bubbled through the mixture for 5 min. To this reaction mixture, PdCl₂(dppf)·DCM (0.104 mg, 0.141 mmol) was added and stirred at 100° C. for 16 h. The reaction mixture was cooled to room temperature, filtered through the Celite pad, and washed with EtOAc (100 mL). The combined filtrate was concentrated under reduced pressure. The resulting crude residue was purified by using MPLC (manually packed SiO₂ cartridge, 100-200 mesh size; 5% EtOAc in hexanes) to obtain boronate 4 as a pale-yellow gum. Yield: 400 mg (50%). LCMS: Calculated for C₉H₁₀BrN is 212.09, Observed: 212.0 [M]⁺ and 214.2 [M+2]⁺

### Step 3:

To a stirred solution of 5 (1.0 g, 2.385 mmol) in MeOH (10 mL), was added p-toluenesulfonic acid monohydrate (0.907 g, 4.77 mmol) at 0° C., and the reaction mixture was stirred at room temperature for 2 h. The reaction was quenched with saturated NaHCO3 solution (100 mL) and extracted with 10% MeOH in DCM (2×100 mL). The combined organic layer was dried over sodium sulfate, filtered, and concentrated under reduced pressure. The crude residue was purified by using MPLC (manually packed cartridge; SiO₂ 100-200 mesh; 5% MeOH in DCM) to obtain 6 as a brown gum. Yield: 0.6 g (72%). LCMS: Calculated for C₁₅H₁₅BrN₂O₂ is 335.20, Observed: 335.1 [M]⁺ and 337.1 [M+2]⁺

### Step 4:

To a solution of 6 (200 mg, 0.597 mmol) in acetonitrile (2 mL) and water (2 mL), were added boronate 4 (232 mg, 0.895 mmol) and K₂CO₃ (247 mg, 1.790 mmol) at room temperature, and a stream of nitrogen gas bubbled through the mixture for 5 min. To this reaction mixture, PdCl₂(dtbpf) (19.44 mg, 0.030 mmol) was added and irradiated in a microwave reactor at 80° C. for 90 min. The reaction mixture was then quenched with water (50 mL) and extracted with 10% MeOH in DCM (2×25 mL). The combined organic layer was dried over anhydrous Na₂SO₄, filtered, and concentrated under reduced pressure. The resulting crude residue was purified by using MPLC (manually packed SiO₂ cartridge, 100-200 mesh size; 7% MeOH in DCM) to obtain Compound 81 as an off-white solid. Yield: 25 mg (11%). LCMS: Calculated for C₂₄H₂₅N₃O₂ is 387.48, Observed: 388.1 [M+1]⁺. 1H-NMR (400 MHz, DMSO-d6): δ 7.59 (d, J=8.40 Hz, 2H), 7.49-7.45 (m, 4H), 7.35 (d, J=0.80 Hz, 1H), 6.84 (s, 1H), 6.78 (d, J=8.40 Hz, 2H), 6.33 (s, 1H, exchanges with D₂O), 5.68 (t, J=6.00 Hz, 1H), 5.52 (t, J=5.60 Hz, 1H, exchanges with D₂O), 5.37 (t, J=5.60 Hz, 1H, exchanges with D₂O), 4.95-4.91 (m, 1H), 3.86 (t, J=6.00 Hz, 2H), 2.37-2.34 (m, 1H), 1.51 (d, J=6.40 Hz, 3H), 0.73-0.69 (m, 2H), 0.41-0.38 (m, 2H). SFC: 93.2%; tR=4.94 min (Column: I Cellulose-Z; Eluents: CO2 and 0.5% isopropyl amine in MeOH). SFC purity=93.2%

### Example A32: Synthesis of Compound 82

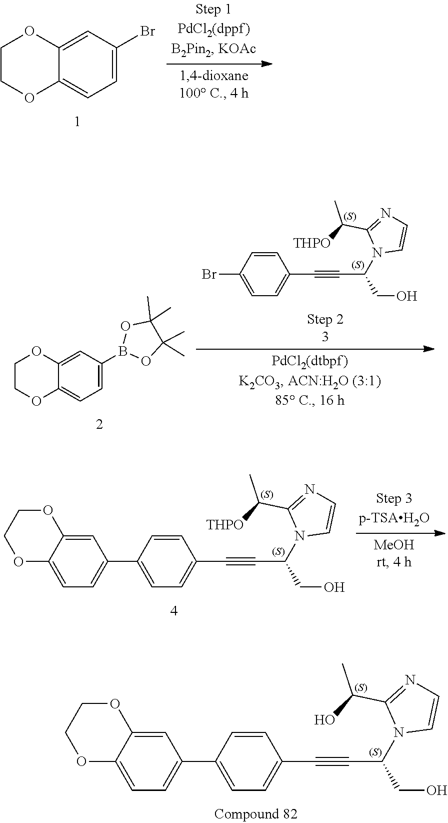

### Step 1:

To a stirred solution of 6-bromo-2,3-dihydrobenzo[b][1,4]dioxine (1, 2.0 g, 9.30 mmol) in 1,4-dioxane (20 mL), were added bis (pinacolato)diboron (2.83 g, 11.16 mmol), and potassium acetate (1.826 g, 18.60 mmol) at room temperature. The reaction mixture was purged with nitrogen for 5 min. To this reaction mixture, PdCl₂(dppf) (0.34 g, 0.465 mmol) was added at room temperature and the resulting reaction mixture stirred at 100° C. for 4 h. The reaction mixture was cooled to room temperature, diluted with DCM (10 mL), filtered through the Celite bed. The Celite bed was washed with DCM (2×20 mL) and the combined filtrate was concentrated under reduced pressure. The crude residue was purified by MPLC (manually packed cartridge; SiO2 230-400 mesh size; 10% EtOAc in hexanes) to afford 2 as an off-white solid. Yield: 1.4 g (57%)

### Step 2:

To the stirred solution of 3 (1.00 g, 2.385 mmol) in acetonitrile (10 mL) and water (3.33 mL), were added 2 (0.688 g, 2.62 mmol) and K2CO3 (0.989 g, 7.15 mmol) at room temperature. The resulting reaction mixture was purged with nitrogen for 5 min. To this reaction mixture, PdCl₂(dtbpf) (0.311 g, 0.477 mmol) was added at room temperature and the resulting reaction mixture was heated to 85° C. and stirred for 16 h. The reaction mixture was cooled to room temperature, quenched with ice water (12 mL) and extracted with 5% MeOH in DCM (3×20 mL). The combined organic extract was washed with brine solution (15 mL), dried over sodium sulphate, filtered, and concentrated under reduced pressure. The crude residue was purified by MPLC (manually packed cartridge; SiO2 230-400 mesh size; 5% MeOH in DCM) to afford 4 as brown sticky solid. Yield: 670 mg (47%). LCMS: Calculated for C₂₈H₃₀N₂O₅ is 474.5. Observed: 475.2 [M+1]⁺

### Step 3:

To the stirred solution of 4 (200 mg, 0.421 mmol) in MeOH (30 mL), was added p-toluenesulfonic acid monohydrate (240 mg, 1.264 mmol) at 0° C. The reaction mixture was allowed to attain room temperature and stirred for 4 h. The reaction mixture was quenched with sat. NaHCO₃ solution (8 mL) at 0° C. and extracted with DCM (3×100 mL). The combined organic extract was washed with sat. NaHCO₃ solution (2×5 mL), brine (10 mL), dried over anhydrous sodium sulphate, filtered, and concentrated under reduced pressure. The resulting crude residue was purified by MPLC (manually packed cartridge, SiO₂ 230-400 mesh size; 5% MeOH in DCM) to afford Compound 82 as an off-white solid. Yield: 45 mg (26%). LCMS: Calculated for C₂₃H₂₂N₂O₄ is 390.1, Observed: 391.2 [M+1]⁺. 1H-NMR (400 MHz, DMSO-d6): δ 7.63 (d, J=8.40 Hz, 2H), 7.50 (d, J=8.40 Hz, 2H), 7.35 (app d, J=1.20 Hz, 1H), 7.20-7.16 (m, 2H), 6.94 (d, J=8.40 Hz, 1H), 6.84 (app d, J=0.80 Hz, 1H), 5.69 (t, J=6.00 Hz, 1H), 5.53 (t, J=5.60 Hz, 1H, exchanges with D2O), 5.37 (d, J=5.60 Hz, 1H, exchanges with D2O), 4.95-4.92 (m, 1H), 4.28 (s, 4H), 3.86 (t, J=6.00 Hz, 2H), 1.51 (d, J=6.40 Hz, 3H). SFC purity=95% (l-Amylose-A_0.5% IPAm in MeOH tR=2.84 min)

### Example A33: Synthesis of Compound 83

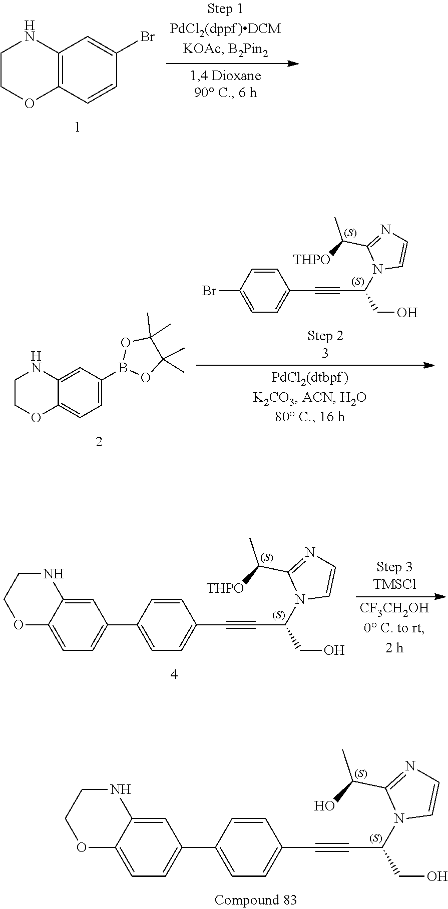

### Step 1:

To a stirred solution of 6-bromo-3,4-dihydro-2H-benzo[b][1,4]oxazine (1, 500 mg, 2.336 mmol) in 1,4-dioxane (10 mL), were added potassium acetate (917 mg, 9.34 mmol) and bis(pinacolato)diboron (1186 mg, 4.67 mmol). The reaction mixture was degassed for 5 min by using nitrogen gas, then PdCl2(dppf)·DCM (171 mg, 0.234 mmol) was added and the degassing continued for 2 min. The reaction mixture was stirred at 90° C. for 6 h. The reaction mixture was filtered through Celite pad, washed with EtOAc (50 mL) and then concentrated under reduced pressure to obtain brown gum. The crude residue was purified by MPLC (using manually packed cartridge; SiO₂ 230-400 mesh; 18% EtOAc in hexane) to obtain boronate 2 as a colourless gum. Yield: 530 mg (86%). LCMS: Calculated for C₁₄H₂OBNO₃ is 261.12, Observed: 262.2 [M+1]⁺

### Step 2:

To a stirred solution of 2 (467 mg, 1.789 mmol) and 3 (500 mg, 1.192 mmol) in acetonitrile (7 mL) and water (7 mL), was added K2CO3 (494 mg, 3.58 mmol). The reaction mixture was degassed for 5 min by using nitrogen gas, then PdCl2(dtbpf) (78 mg, 0.119 mmol) was added under continuous bubbling of nitrogen. The reaction mixture was stirred at 80° C. for 16 h during which time the progress of the reaction was monitored by TLC (DCM:MeOH=1:19). The reaction was quenched with water (50 mL) and extracted with 10% MeOH in DCM (2×50 mL). The combined organic layer was washed with water (50 mL), brine (50 mL), dried over sodium sulfate, filtered and the filtrate concentrated under reduced pressure. The crude residue was purified by MPLC (using manually packed cartridge; SiO₂ 230-400 mesh; 6% MeOH in DCM) to obtain 4 as a pale-brown solid. Yield: 130 mg (23%). LCMS: Calculated for C₂₈H₃₁N₃O₄ is 473.57, Observed: 474.4 [M+1]⁺

### Step 3:

To a solution of 4 (0.13 g, 0.275 mmol) in 2,2,2-trifluoroethanol (2 mL), was added chlorotrimethylsilane (0.052 mL, 0.412 mmol) in a dropwise manner under nitrogen atmosphere. The reaction mixture was stirred at room temperature for 2 h. The reaction was quenched with 10% sat. NaHCO₃ and extracted with 10% MeOH in DCM (2×20 mL). The combined organic layer was washed with water (20 mL), brine (20 mL), dried over sodium sulfate, filtered and the filtrate concentrated under reduced pressure. The crude residue was purified by MPLC (using manually packed cartridge; SiO₂ 230-400 mesh; 5% MeOH in DCM) to obtain Compound 83 as a off-white solid. Yield: 43.5 mg (39%). LCMS: Calculated for C₂₃H₂₃N₃O₃ is 389.45, Observed: 390.2 [M+1]⁺. Single isomer; SFC purity=100%

### Example A34: Synthesis of Compound 84

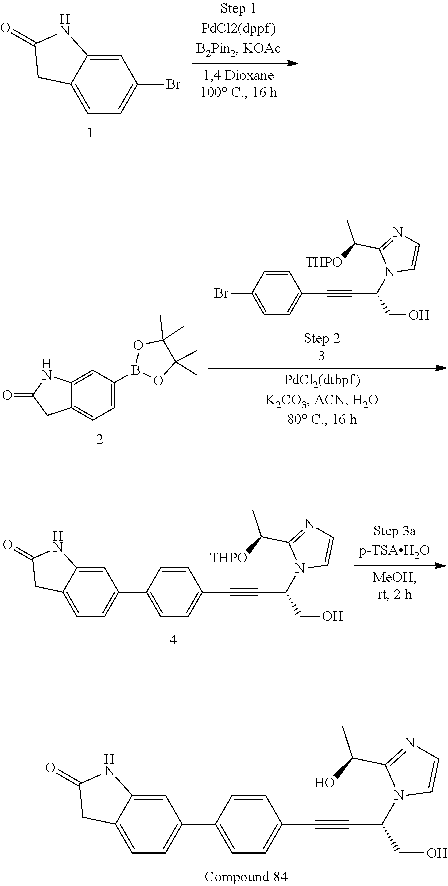

### Step 1:

To a stirred solution of 6-bromooxindole (1, 2 g, 9.43 mmol) and bis(pinacolato)diboron (3.59 g, 14.15 mmol) in dioxane (40 mL), was added potassium acetate (2.78 g, 28.3 mmol) at room temperature following which a stream of nitrogen gas was bubbled through the reaction mixture for 5 min. To this reaction mixture, PdCl2(dppf) (0.690 g, 0.943 mmol) was added and the mixture was stirred at 100° C. for 16 h. The reaction was quenched with water (100 mL) and extracted with EtOAc (50 mL×2). The combined organic layer was washed with brine (50 mL), dried over anhydrous Na₂SO₄, filtered, and concentrated under reduced pressure to get the crude product. The crude residue was purified by MPLC (manually packed cartridge, SiO₂ 230-400 mesh; 30% EtOAc in hexanes) to obtain 2 as a pale yellow solid. Yield: 2.5 g (51%). UPLC-MS: Calculated for C14H18BNO3 is 259.1, Observed: 260.1 [M+1]⁺

### Step 2:

To a stirred solution of 2 (2.5 g, 9.65 mmol) in water (15 mL) and acetonitrile (15 mL), were added 3 (2.7 g, 6.44 mmol) and K2CO3 (2.67 g, 19.32 mmol) at room temperature following which a stream of nitrogen gas was bubbled through the mixture for 5 min. To this reaction mixture, PdCl2(dtbpf) (0.420 g, 0.644 mmol) was added and the bubbling of nitrogen continued for another 2 min. The resulting reaction mixture was stirred at 80° C. for 16 h. The reaction was quenched with water (100 mL) and extracted with 10% MeOH in DCM (80 mL×2). The combined organic layer was washed with brine (30 mL), dried over anhydrous Na₂SO₄, filtered, and concentrated under reduced pressure to get the crude product. The crude product was purified by MPLC (manually packed cartridge, SiO₂ 230-400 mesh; 5% MeOH in DCM) to obtain 4 as a brown color solid. Yield: 1.1 g (355%). UPLC-MS: Calculated for C28H29N3O4 is 471.55, Observed: 472.2 [M+1]⁺

### Step 3:

To a solution of 4 (0.1 g, 0.212 mmol) in MeOH (5 mL), p-toluenesulfonic acid monohydrate (0.121 g, 0.636 mmol) was added at room temperature and the resulting reaction mixture stirred at room temperature for 2 h. The volatiles were concentrated under reduced pressure; the crude residue was basified with sat.NaHCO3 solution (10 mL). The aqueous layer was extracted with 10% MeOH in DCM (10 mL×2). The combined organic extract was dried over anhydrous sodium sulfate, filtered, and concentrated under reduced pressure. The resulting crude residue was purified by preparative HPLC (Column: X-BRIDGE C8 (19×150 mm) 5 μm; Eluents:10 mM ammonium bicarbonate in water and acetonitrile) to obtain Compound 84 as a white solid. Yield: 32 mg (39%). UPLC-MS: Calculated for C23H21N3O3 is 387.44, Observed: 388.2 [M+1]⁺. 1H-NMR (400 MHz, DMSO-d6): δ 10.48 (s, 1H), 7.64 (d, J=8.40 Hz, 2H), 7.54 (d, J=8.40 Hz, 2H), 7.36 (d, J=1.20 Hz, 1H), 7.31-7.24 (m, 2H), 7.04 (s, 1H), 6.84 (d, J=1.20 Hz, 1H), 5.70 (t, J=6.00 Hz, 1H), 5.53 (t, J=5.60 Hz, 1H, exchanges with D2O), 5.37 (d, J=5.60 Hz, 1H, exchanges with D2O), 5.37-4.91 (m, 1H), 3.87 (t, J=5.60 Hz, 2H), 3.52 (s, 2H), 1.51 (d, J=6.40 Hz, 2H). SFC=>98% (R,R-Whelk_0.5% ⁱPrNH₂ in MeOH_40; tR=2.37 min)

### Example A35: Synthesis of Compound 85

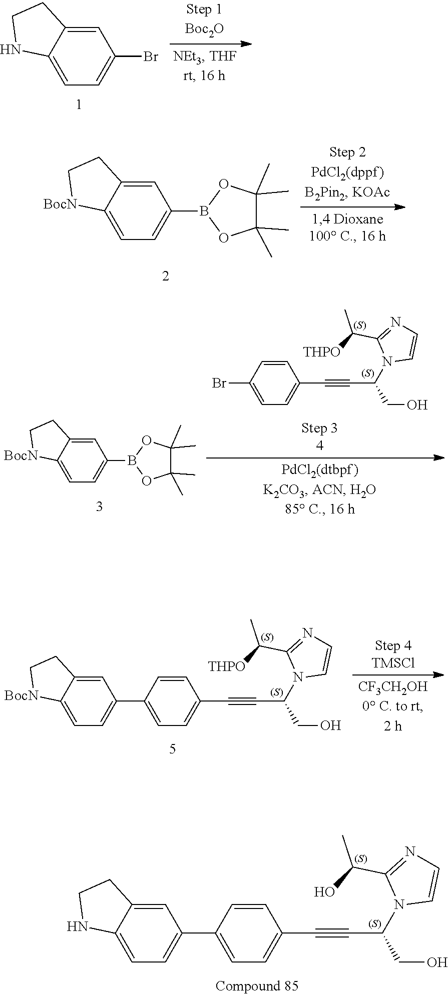

### Step-1:

To a stirred solution of 5-bromoindoline (1, 5 g, 25.2 mmol) in THF (50 mL), were added triethylamine (10.56 mL, 76 mmol) and di-tert-butyl dicarbonate (7.03 mL, 30.3 mmol) at room temperature. The reaction mixture was stirred at room temperature for 16 h after which it was quenched with water (100 mL) and extracted with EtOAc (2×100 mL). The combined organic extract was washed with brine solution (100 mL), dried over anhydrous Na₂SO₄, filtered, and concentrated under pressure to afford 2 as a white solid. Yield=6.8 g (90%). LC-MS: Calculated C13H16BrNO2 is 297.04, Observed: 198.2 [M-Boc]⁺ and 200.0 [(M-Boc)+2]⁺

### Step-2:

To a stirred solution of 2 (4 g, 13.41 mmol) in dioxane (70 mL), were added potassium acetate (2.376 g, 40.2 mmol) and bis(pinacolato)diboron (5.11 g, 20.12 mmol) at room temperature and the resulting mixture purged with nitrogen for 5 min. To this reaction mixture, PdCl2(dppf) (0.982 g, 1.341 mmol) was added and the purging continued for 2 min. The reaction mixture was stirred at 100° C. for 16 h, after which time it was cooled to room temperature and quenched with water (50 mL) and extracted using EtOAc (2×150 mL). The combined organic extract was washed with brine (50 mL), dried over anhydrous Na₂SO₄, filtered, and concentrated under reduced pressure. The resulting crude mass was purified by using MPLC (manually packed SiO2 cartridge, 230-400 mesh; 90% EtOAc in hexanes) to afford boronate 3 as a yellow solid. Yield=1.8 g (35%). LC-MS: Calculated C19H28BNO4 is 345.21, Observed: 246.3 [(M-Boc)+1]⁺

### Step-3:

To a stirred solution of 4 (2.4 g, 5.72 mmol) in acetonitrile (50 mL) and water (5 mL), were added boronate 3 (1.976 g, 5.72 mmol) and K2CO3 (2.373 g, 17.17 mmol). The reaction mixture was purged with nitrogen for 5 min. To this reaction mixture, PdCl2(dtbpf) (1.119 g, 1.717 mmol) was added and the purging continued for another 2 min. The reaction mixture was stirred at 85° C. for 16 h. To the reaction cooled to room temperature, water (50 mL) was added and extracted with EtOAc (2×250 mL). The combined organic extract was washed with brine (50 mL), dried over anhydrous Na₂SO₄, filtered, and concentrated under reduced pressure. The resulting crude mass was purified by using MPLC (manually packed SiO₂ cartridge, 230-400 mesh size; 5% MeOH in EtOAc) to afford 5 as a brown solid. Yield=1.2 g (34%). LC-MS: Calculated C₃₃H₃₉N₃O₅ is 557.69, Observed: 558.1 [M+1]⁺

### Step-4:

To a stirred solution of 5 (500 mg, 0.897 mmol) in 2,2,2-trifluoroethanol (10 mL), was added TMSCl (0.171 mL, 1.345 mmol) at 0° C., and the reaction mixture stirred at room temperature for 2 h. The reaction mixture was quenched with 10% sodium bicarbonate solution (10 mL) and extracted with DCM (50 mL). The combined organic extract was dried over anhydrous Na₂SO₄, filtered, and concentrated under reduced pressure. The crude mass, thus obtained, was purified by reversed phase preparative HPLC (Column: Shimpack-c18 (150*20 mm) 5 μm; Eluents: 10 mM ammonium bicarbonate in water and ACN) to afford Compound 85 as an off-white solid. Yield: 61.88 mg (16%). LC-MS: Calculated C₂₃H₂₃N₃O₂ is 373.45, Observed: 374.2 [M+1]⁺. 1H-NMR (400 MHz, DMSO-d6): δ 7.57 (d, J=8.40 Hz, 2H), 7.45 (d, J=8.40 Hz, 2H), 7.39 (s, 1H), 7.35 (d, J=0.80 Hz, 1H), 7.28 (d, J=8.00 Hz, 1H), 6.84 (d, J=0.80 Hz, 1H), 6.55 (d, J=8.00 Hz, 1H), 5.74 (br s, 1H, exchanges with D2O), 5.68 (t, J=6.00 Hz, 1H), 5.52 (t, J=5.60 Hz, 1H, exchanges with D2O), 5.36 (d, J=5.60 Hz, 1H, exchanges with D2O), 4.95-4.92 (m, 1H), 3.86 (t, J=6.00 Hz, 2H), 3.47 (td, J=1.20, 8.60 Hz, 2H), 2.97 (t, J=8.80 Hz, 2H), 1.51 (d, J=6.40 Hz, 3H). SFC: 94.2%; tR=2.12 min (Column: R,R-Whelk; Eluents: CO2 and 0.5% isopropyl amine in MeOH)

### Example A36: Synthesis of Compound 86

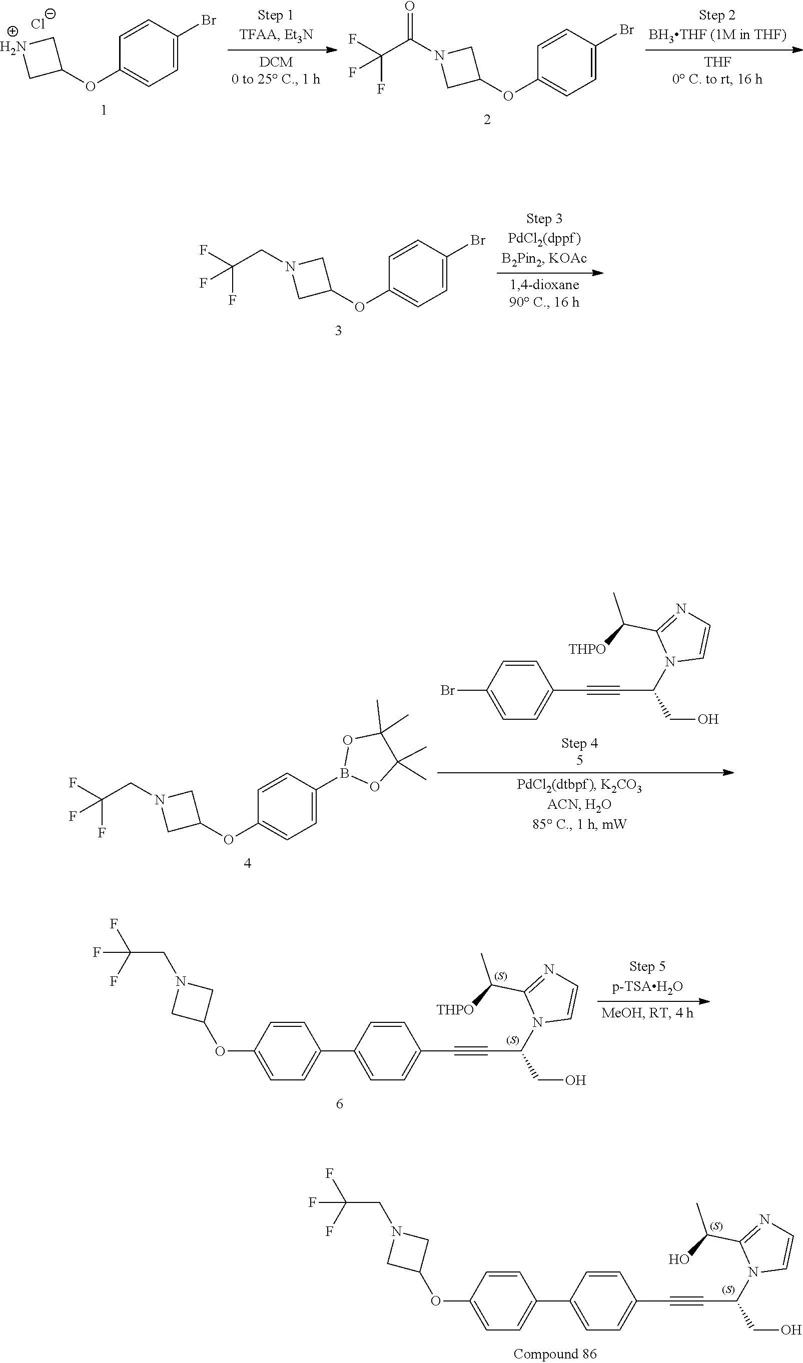

### Step 1:

To the stirred solution of 1 (5.0 g, 18.90 mmol) in DCM (50 mL), were added TFAA (4.00 mL, 28.4 mmol) and TEA (3.95 mL, 28.4 mmol) at 0° C., and the resulting reaction mixture stirred at room temperature for 1 h. The reaction was quenched with ice cold water (30 mL) and extracted with DCM (30 mL×2). The combined organic extract was washed with brine solution (25 mL), dried over anhydrous sodium sulphate, filtered and concentrated under reduced pressure. The crude residue thus obtained was purified by using MPLC (manually packed SiO2 cartridge; 230-400 mesh size; 5% EtOAc in hexanes) to afford 2 as a colorless liquid. Yield: 5.8 g (80%).

### Step 2:

To the stirred solution of 2 (5.8 g, 17.90 mmol) in THF (60 mL), was added BH₃-THF (1.0 M in THF, 53.7 mL, 53.7 mmol) at 0° C. and the resulting reaction mixture stirred at room temperature for 16 h. The reaction mixture was then quenched by slow addition (drops!) of MeOH (40 mL) at 0° C. The resulting reaction mixture was allowed to reach room temperature and then heated to 50° C. for 10 min. The reaction mixture was concentrated under reduced pressure to afford a crude residue which was purified by using MPLC (manually packed SiO₂ cartridge; 230-400 mesh size; 8% EtOAc in hexanes) to afford 3 as pale-yellow liquid. Yield: 4.4 g (71%). LCMS: Calculated for C11H11BrF3NO is 310.1, Observed: 310.0 [M]⁺ and 312.0 [M+2]⁺

### Step 3:

To a stirred solution of 3 (3.8 g, 12.25 mmol) in 1,4-dioxane (40 mL), were added potassium acetate (2.405 g, 24.51 mmol) and bis(pinacolato)diboron (4.67 g, 18.38 mmol) at room temperature, and the reaction mixture purged with nitrogen for 5 min. To this reaction mixture, PdCl2(dppf) (0.448 g, 0.613 mmol) was added and the purging continued for 5 min. The reaction mixture was heated to 90° C. and stirred for 16 h. The reaction mixture was cooled to room temperature, filtered through a Celite pad. The pad was washed with EtOAc (40 mL×2). The combined filtrate was washed with brine solution (20 mL), dried over anhydrous sodium sulphate, filtered, and concentrated under reduced pressure. The crude residue thus obtained was purified by using MPLC (manually packed SiO2 cartridge; 230-400 mesh size; 5% EtOAc in hexanes) to afford 4 as an off-white solid. Yield: 4.3 g (84%). LCMS: Calculated for C17H23BF3NO3 is 357.18, observed: 358.2 [M+1]⁺

### Step 4:

To s solution of 5 (250 mg, 0.596 mmol) in a mixture of acetonitrile (2 mL) and water (2 mL), were added K2CO3 (247 mg, 1.789 mmol) and 4 (256 mg, 0.715 mmol) at room temperature and the reaction mixture purged with nitrogen for 5 min. To this reaction mixture, PdCl2(dtbpf) (38.9 mg, 0.060 mmol) was added and the purging continued for 5 min. The reaction mixture was irradiated at 85° C. for 1 h in a microwave reactor. The reaction was quenched with water (2 mL) and extracted with 5% MeOH in DCM (10 mL×2). The combined organic extract was washed with brine (5 mL), dried over anhydrous sodium sulphate, filtered, and concentrated under reduced pressure. The crude residue, thus obtained, was purified by using MPLC (manually packed SiO2 cartridge; 230-400 mesh size; 5% MeOH in DCM) to get 6 as brown sticky liquid. Yield: 230 mg (66%). LCMS: Calculated for C31H34F3N3O4 is 569.63, observed: 570.2 [M+1]⁺

### Step 5:

To the stirred solution of 6 (115 mg, 0.202 mmol) in MeOH (15 mL), was added p-TSA·H2O (115 mg, 0.606 mmol) at room temperature and the reaction mixture stirred at room temperature for 4 h. One more batch was performed with 115 mg of 6. Both batches were quenched individually with sat. NaHCO3 solution (5 mL) at 0° C. The resulting suspensions were mixed for work-up and purification. The combined suspension was extracted with DCM (50 mL×3). The combined organic extract was washed with saturated NaHCO₃ solution (3×5 mL), brine solution (15 mL), dried over anhydrous sodium sulphate, filtered, and concentrated under reduced pressure. The crude residue, thus obtained, was purified by reverse phase preparative HPLC (Column: Shimpack C18 (150*20) 5 μm; Eluents: 10 mM ammonium bicarbonate in water and acetonitrile) to afford Compound 86 as an off-white solid. Yield: 48 mg (combined yield of two batches). LCMS: Calculated for C26H26F3N3O3 is 485.5, Observed: 486.4 [M+1]⁺. 1H-NMR (400 MHz, DMSO-d6): δ 7.66-7.62 (m, 4H), 7.51 (d, J=8.40 Hz, 2H), 7.35 (d, J=1.20 Hz, 1H), 6.93 (d, J=8.80 Hz, 2H), 6.84 (d, J=1.20 Hz, 1H), 5.69 (t, J=6.00 Hz, 1H), 5.53 (t, J=6.00 Hz, 1H, exchanges with D2O), 5.37 (d, J=5.60 Hz, 1H, exchanges with D2O), 4.95-4.92 (m, 2H), 3.93-3.85 (m, 4H), 3.35-3.30 (m, 4H), 1.51 (d, J=6.40 Hz, 3H). 19F (MHz, DMSO-d6): δ −69.86. SFC: 100%; tR=1.82 min (Column: LUX-I Amylose-3; Eluents: 0.5% isopropyl amine in MeOH). Single isomer with SFC purity=100%.

### Example A37: Synthesis of Compound 87

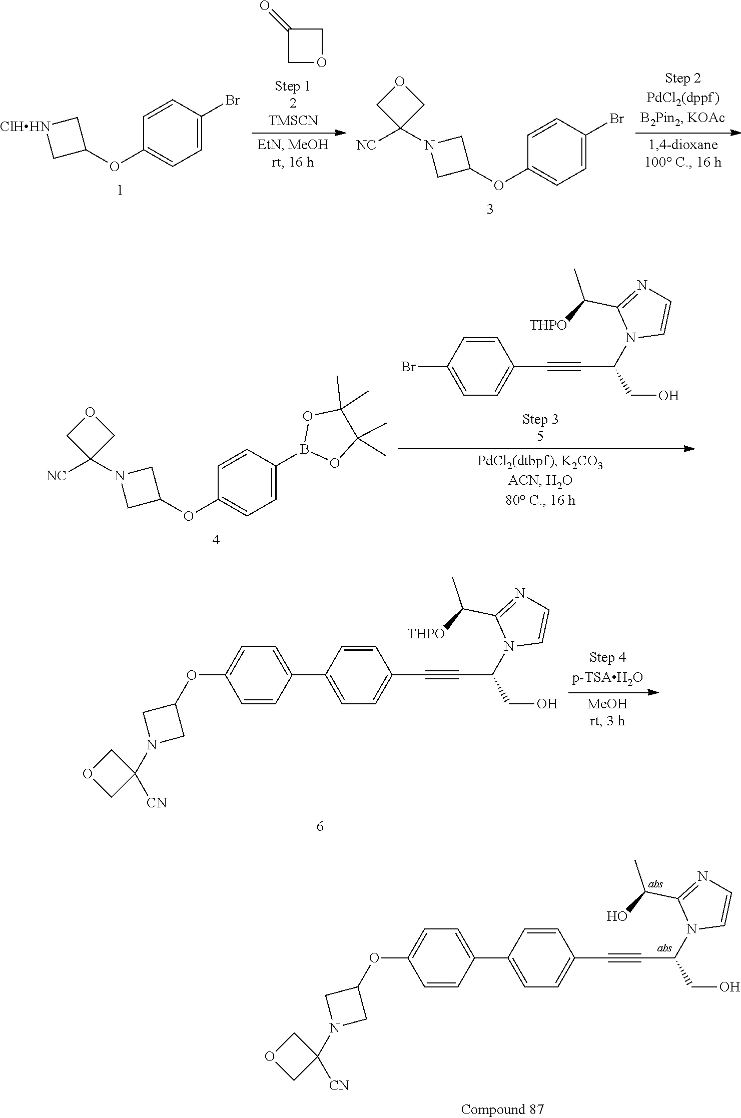

### Step 1:

To a stirred solution of 3-(4-bromophenoxy) azetidine. HCl (1; 5.22 g, 22.9 mmol) in MeOH (5 mL), was added triethylamine (2.9 mL, 20.82 mmol) under nitrogen. The resulting mixture was stirred for 15 min, concentrated under reduced pressure. The resulting residue was taken in MeOH (30 mL). To this mixture, oxetan-3-one (2, 1.5 g, 20.82 mmol) and acetic acid (5.96 mL, 104 mmol) were added and stirred for 1 h at room temperature. Then, trimethylsilyl cyanide (2.79 mL, 20.82 mmol) was added and stirring continued for 16 h. The volatiles were concentrated under reduced pressure. To the resulting residue, water (50 mL) was added. This was extracted with EtOAc (2×100 mL). The combined organic layer was concentrated under reduced pressure, obtained crude product was purified by MPLC (using manually packed SiO₂ cartridge, 100-200 mesh; 22% EtOAc in n-hexanes) to get 3 as a white solid. Yield: 2.5 g

### Step 2:

To a stirred solution of 3 (160 mg, 0.518 mmol) in 1,4-dioxane (5 mL), were added potassium acetate (152 mg, 1.553 mmol), bis(pinacolato)diboron (197 mg, 0.776 mmol) at room temperature. The resulting mixture was purged with nitrogen for 10 min. To this reaction mixture, PdCl₂(dppf) (37.9 mg, 0.052 mmol) was added and the purging continued for 2 min. The reaction mixture was stirred at 100° C. for 16 h. The reaction mixture was cooled to room temperature, quenched with water (20 mL) and extracted with EtOAc (20 mL). The combined organic layer was washed with brine solution (20 mL), dried over anhydrous sodium sulphate, filtered and concentrated under reduced pressure. The crude mass, thus obtained, was purified by MPLC (using manually packed SiO₂ cartridge, 100-200 mesh; 10% EtOAc in hexanes) to afford 4 as a white solid. Yield: 130 mg (68%). LC-MS: Calculated for C₁₉H₂₅BN₂O₄ is 356.2, Observed: [M+H]⁺=357.2

### Step 3:

To a stirred solution of 4 (130 mg, 0.365 mmol) in acetonitrile (5 mL) and water (0.05 mL), were added 5 (230 mg, 0.547 mmol) and K₂CO₃ (151 mg, 1.095 mmol). The resulting mixture was purged with nitrogen for 5 min. To this reaction mixture, was added PdCl₂(dtbpf) (11.88 mg, 0.018 mmol) was added and the purging continued with nitrogen for another 2 min. The reaction mixture was stirred at 80° C. for 16 h. The reaction mixture was quenched with water (20 mL), extracted with 10% MeOH in DCM (2×20 mL). The combined organic layer was washed with brine solution (10 mL), dried over anhydrous sodium sulphate, filtered and concentrated under reduced pressure. The resulting crude product was purified by MPLC (using manually packed SiO₂ cartridge, 100-200 mesh size; 0-10% MeOH in DCM) to afford 6 as a brown gummy. The product showed 72% purity by LCMS; product was taken to the next step. Yield: 110 mg (38%). LC-MS: Calculated for C₃₃H₃₆N₄O₅ is 568.67, Observed: [M+H]⁺=569.3

### Step-4:

To a stirred solution of 6 (110 mg, 0.193 mmol) in MeOH (5 mL), was added p-toluene sulfonic acid monohydrate (110 mg, 0.58 mmol) at 0° C., and the reaction mixture stirred at room temperature for 3 h. The volatiles were concentrated under reduced pressure, the resulting residue was basified with saturated NaHCO₃ solution (10 mL) and extracted with 10% MeOH in DCM (2×10 mL). The combined organic layer was washed with brine (10 mL), dried over anhydrous sodium sulphate, filtered and concentrated under reduced pressure. The crude compound was purified by reversed phase preparative HPLC (Column: Shim pack C18 (150*20) mm, 5 μm; Eluents: 10 mM ammonium bicarbonate in water and acetonitrile) to obtain Compound 87 as a white solid. Yield: 21 mg (22%). LC-MS: Calculated for C₂₈H₂₈N₄O₄ is 484.5, Observed: [M+H]⁺=485.2. 7.67-7.64 (m, 4H), 7.52 (d, J=8.40 Hz, 2H), 7.36 (d, J=0.80 Hz, 1H), 6.98 (d, J=8.80 Hz, 2H), 6.84 (s, 1H), 5.70 (t, 1H), 5.52 (d, J=5.60 Hz, 1H, exchanges with D2O), 5.37 (d, J=5.60 Hz, 1H, exchanges with D2O), 5.01-4.92 (m, 2H), 4.80 (d, J=7.20 Hz, 2H), 4.43 (d, J=7.20 Hz, 2H), 3.92-3.85 (m, 4H), 3.29-3.26 (m, 2H), 1.51 (d, J=6.40 Hz, 3H). l-cellulose B_0.5% IPAm in MeOH tR=3.12 min

### Example A38: Synthesis of Compound 88

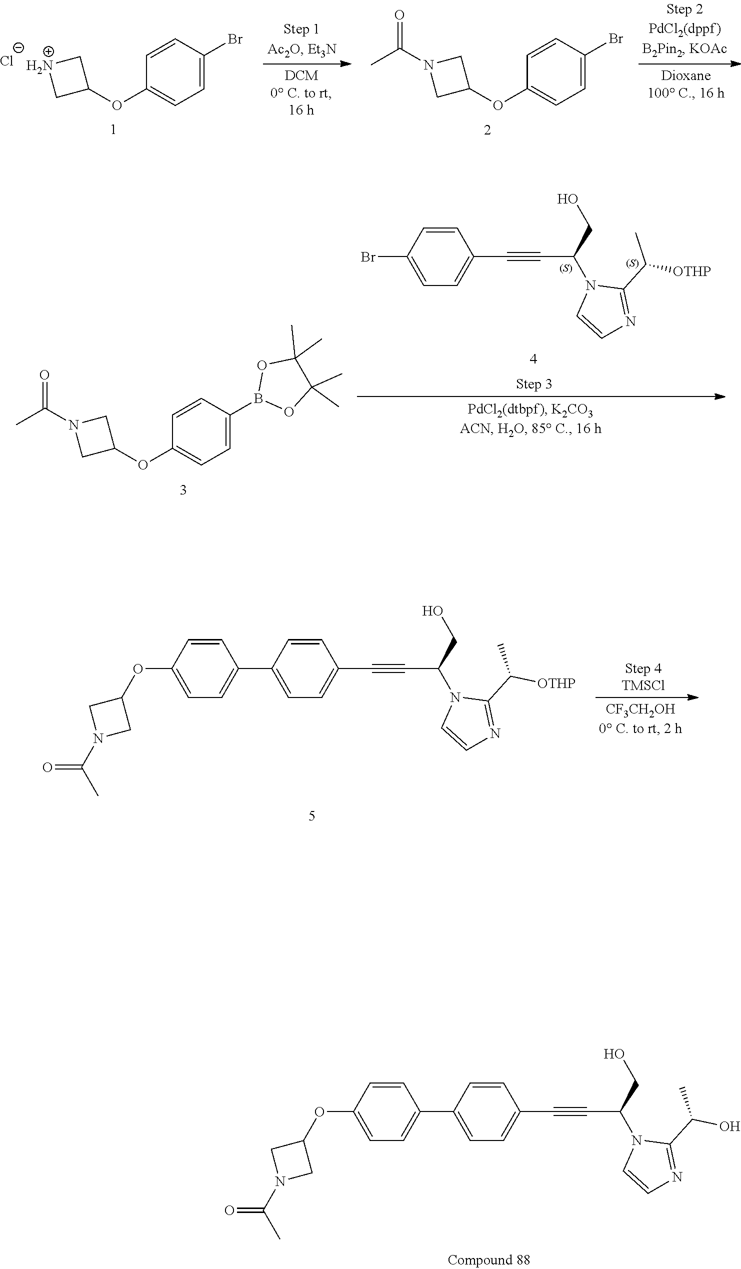

### Step-1:

To a stirred solution of 1 (4.0 g, 17.54 mmol) in DCM (200 mL), were added triethylamine (7.33 mL, 52.6 mmol) and acetic anhydride (3.31 mL, 35.1 mmol) at 0° C. The reaction mixture was stirred at room temperature for 16 h. The reaction was quenched with water (100 mL) and extracted with DCM (2×100 mL). The combined organic extract was washed with brine solution (100 mL) and dried over anhydrous Na₂SO₄, filtered and concentrated under reduced pressure. The resulting crude mass was purified by MPLC (manually packed SiO2 cartridge, 100-200 mesh size; 10-20% EtOAc in hexanes) to afford 2 as an off-white solid. Yield: 3.28 g (55%). LC-MS: Calculated for C₁₁H₁₂BrNO₂ is 270.13, Observed: 270. [M]⁺ and 272.0 [M+2]⁺

### Step-2:

To a stirred solution of 2 (2.8 g, 10.37 mmol) in dioxane (60 mL), were added potassium acetate (3.05 g, 31.1 mmol) and bis(pinacolato)diboron (3.95 g, 15.55 mmol) at room temperature. The resulting mixture was purged with nitrogen for 5 min. To this reaction mixture, PdCl₂(dppf) (0.76 g, 1.037 mmol) was added, the purging continued for another 2 min and the resulting reaction mixture stirred at 100° C. for 16 h. The reaction mixture was then cooled to room temperature. The inorganic solids were filtered through a Celite pad and washed with EtOAc (2×500 mL). The combined filtrate was concentrated under reduced pressure. The crude mass, thus obtained, was purified by MPLC (manually packed SiO₂ cartridge, 230-400 mesh size; 10-20% EtOAc in hexanes) to afford 3 as a light brown viscous liquid. Yield: 2.9 g (79%) LC-MS: Calculated for C₁₇H₂₄BNO₄ is 317.19, Observed: 318.4 [M+1]⁺

### Step-3:

To a stirred solution of 4 (1.8 g, 4.29 mmol) in acetonitrile (40 mL) and water (10 mL), were added 3 (2.04 g, 6.44 mmol) and potassium carbonate (1.78 g, 12.88 mmol) at room temperature, and the reaction mixture purged with nitrogen for 5 min. To this reaction mixture, PdCl2(dtbpf) (0.84 g, 1.29 mmol) was added and the purging continued for another 2 min. The resulting reaction mixture was stirred at 85° C. for 16 h. After the reaction was cooled to ambient temperature; water (50 mL) was added and extracted with EtOAc (2×100 mL). The combined organic extract was washed with brine (50 mL), dried over anhydrous Na₂SO₄, filtered and concentrated under reduced pressure. The resulting crude mass was purified by MPLC (manually packed SiO₂ cartridge, 100-200 mesh size; 10% MeOH in EtOAc) to afford 5 as a brown solid. Yield: 1.5 g (53%). LC-MS: Calculated for C₃₁H₃₅N₃O₅ is 529.64, Observed: 530.2 [M+1]⁺

### Step-4:

To a stirred solution of 5 (1.5 g, 2.83 mmol) in 2,2,2-trifluoroethanol (20 mL), was added TMSCl (0.543 mL, 4.25 mmol) at 0° C. and the reaction mixture stirred at room temperature for 2 h. The volatiles in the reaction mixture were then removed under reduced pressure. The crude mass, thus obtained, was purified by reverse phase chromatography (Column: Redisep Gold; C18 SiO₂; Eluents: 10 mM formic acid in water: ACN). The resulting product was further re-purified by reversed phase chromatography (Column: Redisep Gold; C18 SiO₂; Eluents: 10 mM ammonium bicarbonate in water: ACN) to afford Compound 88 as a white solid. Yield: 65 mg (5%). LC-MS: Calculated for C₂₆H₂₇N₃O₄ is 445.52, Observed: 446.2 [M+1]⁺. 1H-NMR (400 MHz, DMSO-d6): δ 7.67-7.63 (m, 4H), 7.52 (d, J=8.40 Hz, 2H), 7.36 (app d, J=1.20 Hz, 2H), 6.95 (d, J=8.80 Hz, 1H), 6.84 (app d, J=1.20 Hz, 1H), 5.69 (t, J=6.00 Hz, 1H), 5.53 (t, J=5.60 Hz, 1H, exchanges with D2O), 5.37 (d, J=5.60 Hz, 1H, exchanges with D2O), 5.10-5.06 (m, 1H), 4.96-4.93 (m, 1H), 4.60-4.56 (m, 1H), 4.33-4.28 (m, 1H), 4.12-4.09 (m, 1H), 3.87 (t, J=6.00 Hz, 2H), 3.79-3.32 (m, 1H), 1.82 (s, 3H), 1.51 (d, J=6.40 Hz, 3H). Single isomer; SFC purity=98% (l-cellulose Z_0.5% IPAm in MeOH; tR=3.96 min).

### Example A39: Synthesis of Compound 89

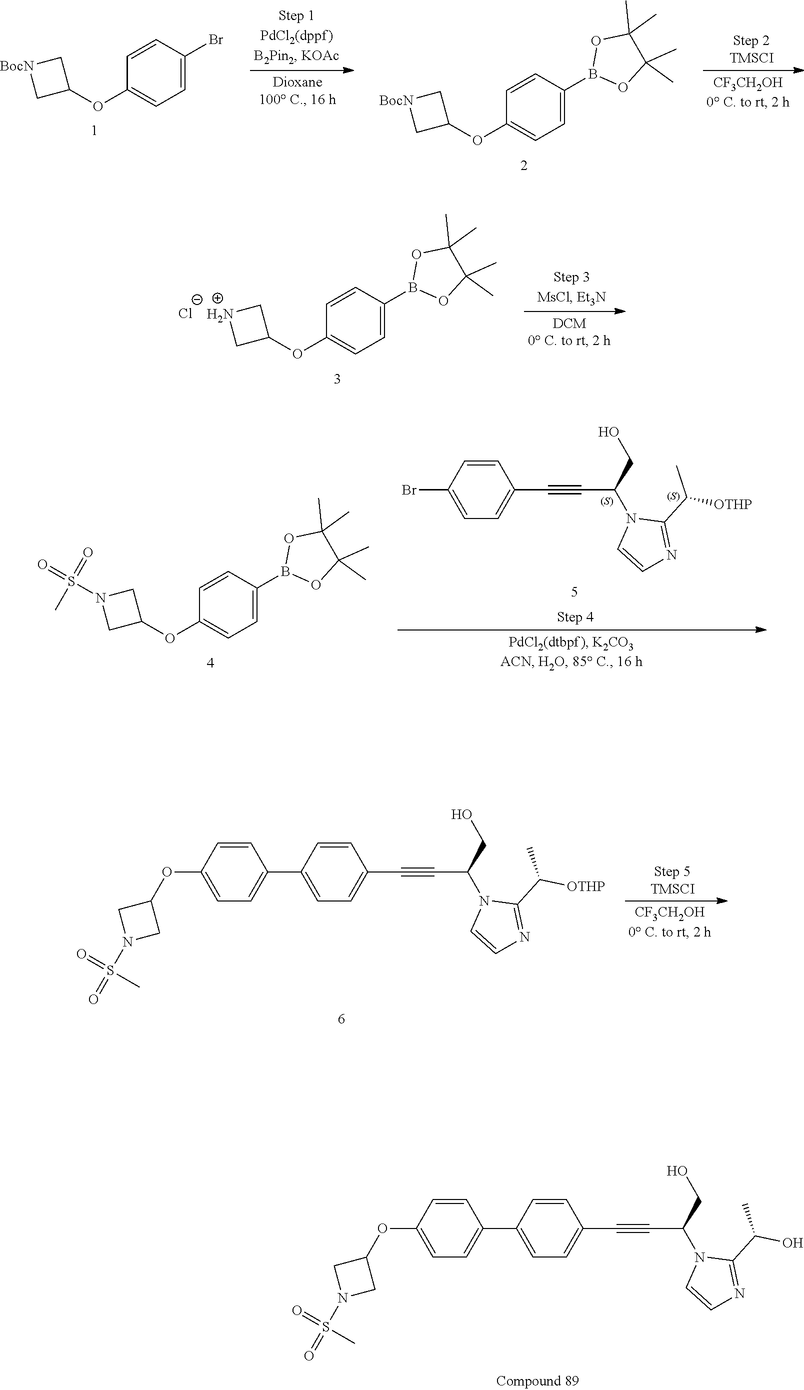

### Step-1:

To a stirred solution of 1 (5 g, 15.23 mmol) in dioxane (200 mL), were added potassium acetate (4.49 g, 45.7 mmol) and bis(pinacolato)diboron (5.80 g, 22.85 mmol) at room temperature. The resulting mixture was purged with nitrogen for 10 min. To this reaction mixture, PdCl2(dppf) (1.115 g, 1.523 mmol) was added and the purging continued for 2 min. The reaction mixture was then stirred at 100° C. for 16 h. The reaction was cooled to ambient temperature and filtered through a Celite bed. The Celite bed was washed with EtOAc (200 mL) and the combined filtrate concentrated under reduced pressure to get a crude residue. The residue was purified by MPLC (using manually packed SiO2 cartridge, 230-400 mesh; 30% EtOAc in hexanes) to afford boronate 2 as a white solid. Yield=5.2 g (48%). LC-MS: Calculated for C₂₀H₃₀BNO₅ is 375.27, Observed: 276.2 [M-Boc+1]⁺

### Step-2:

To a stirred solution of 2 (5.2 g, 13.86 mmol) in 2,2,2-trifluoroethanol (110 mL), was added TMSCl (4.43 mL, 34.6 mmol) at 0° C. The reaction mixture was stirred at room temperature for 2 h. The volatiles in the reaction mixture were removed under reduced pressure to afford 3 as yellow liquid. The crude material was taken to the next step without further purification. Yield=4.5 g (90%). LC-MS: Calculated C₁₅H₂₃BNO₃⁺ is for free base is 276.18, Observed: 276.1 [M]⁺

### Step-3:

To a stirred solution of 3 (4 g, 12.84 mmol) in DCM (70 mL), were added TEA (5.41 mL, 38.5 mmol) and methanesulfonyl chloride (1.590 mL, 20.54 mmol) at 0° C. and the reaction mixture stirred at room temperature for 2 h. The reaction was quenched with water (30 mL) and extracted with DCM (50 mL×2). The combined organic extract was washed with brine solution (50 mL), dried over anhydrous Na₂SO₄, filtered and concentrated under reduced pressure. The resulting crude mass was purified by MPLC (using manually packed SiO2 cartridge, 230-400 mesh; 45% EtOAc in hexanes) to afford 4 as a yellow solid. Yield: 2.5 g (41%). LC-MS: Calculated C₁₆H₂₄BNO₅S is 353.24, Observed: 354.2 [M+1]⁺

### Step-4:

To a stirred solution of 5 (2.5 g, 5.96 mmol) in acetonitrile (40 mL) and water (10 mL), were added boronate 4 (2.53 g, 7.15 mmol) and K2CO3 (2.472 g, 17.89 mmol). The reaction mixture was purged with nitrogen for 5 min. To this reaction mixture, PdCl2(dtbpf) (1.166 g, 1.789 mmol) was added and the purging continued for another 2 min. The reaction mixture was then stirred at 85° C. for 16 h. To the reaction mixture cooled to ambient temperature, water (50 mL) was added and extracted with EtOAc (2×100 mL). The combined organic extract was washed with brine (50 mL), dried over anhydrous Na₂SO₄, filtered and concentrated under reduced pressure. The resulting crude mass was purified by MPLC (using manually packed SiO₂ cartridge, 230-400 mesh size; 10% MeOH in EtOAc) to afford 6 as brown solid. Yield=1.5 g (27%). LC-MS: Calculated C₃₀H₃₅N₃O₆S is 565.68, Observed: 566.2 [M+1]⁺

### Step-5:

To a stirred solution of 6 (1 g, 1.768 mmol) in 2,2,2-trifluoroethanol (20 mL), was added TMSCl (0.215 mL, 1.768 mmol) at 0° C. and the reaction mixture stirred at room temperature for 2 h. The reaction mixture was quenched with 10% sodium bicarbonate solution (10 mL) and extracted with DCM (100 mL). The combined organic extract was dried over anhydrous Na₂SO₄, filtered and concentrated under reduced pressure. The crude mass, thus obtained, was purified by reversed phase preparative HPLC (Column: Shimpack-c18 (150*20 mm) 5 μm; Eluents: 10 mM ammonium bicarbonate in water and ACN) to afford Compound 89 as an off-white solid. Yield: 75 mg (9%). LC-MS: Calculated C₂₅H₂₇N₃O₅S is 481.56, Observed: 482.2 [M+1]⁺. 1H-NMR (400 MHz, DMSO-d6): δ 7.66 (dd, J=2.00, 8.40 Hz, 4H), 7.52 (d, J=8.40 Hz, 2H), 7.36 (app d, J 1.20 Hz, 1H), 6.97 (dd, J=2.00, 6.80 Hz, 2H), 6.84 (app d, J=1.20 Hz, 1H), 5.69 (t, J=6.00 Hz, 1H), 5.53 (t, 1H, exchanges with D2O), 5.37 (d, J=5.60 Hz, 1H, exchanges with D2O), 5.13-5.07 (m, 1H), 4.96-4.91 (m, 1H), 4.34 (dd, J=6.40, 9.60 Hz, 2H), 3.95 (dd, J=4.80, 9.60 Hz, 2H), 3.87 (t, J=6.00 Hz, 2H), 3.08 (s, 3H), 1.51 (d, J=6.80 Hz, 3H). SFC purity=96.6% (l-cellulose Z_0.5% IPAm in MeOH tR=4.27 min)

### Example A40: Synthesis of Compound 90

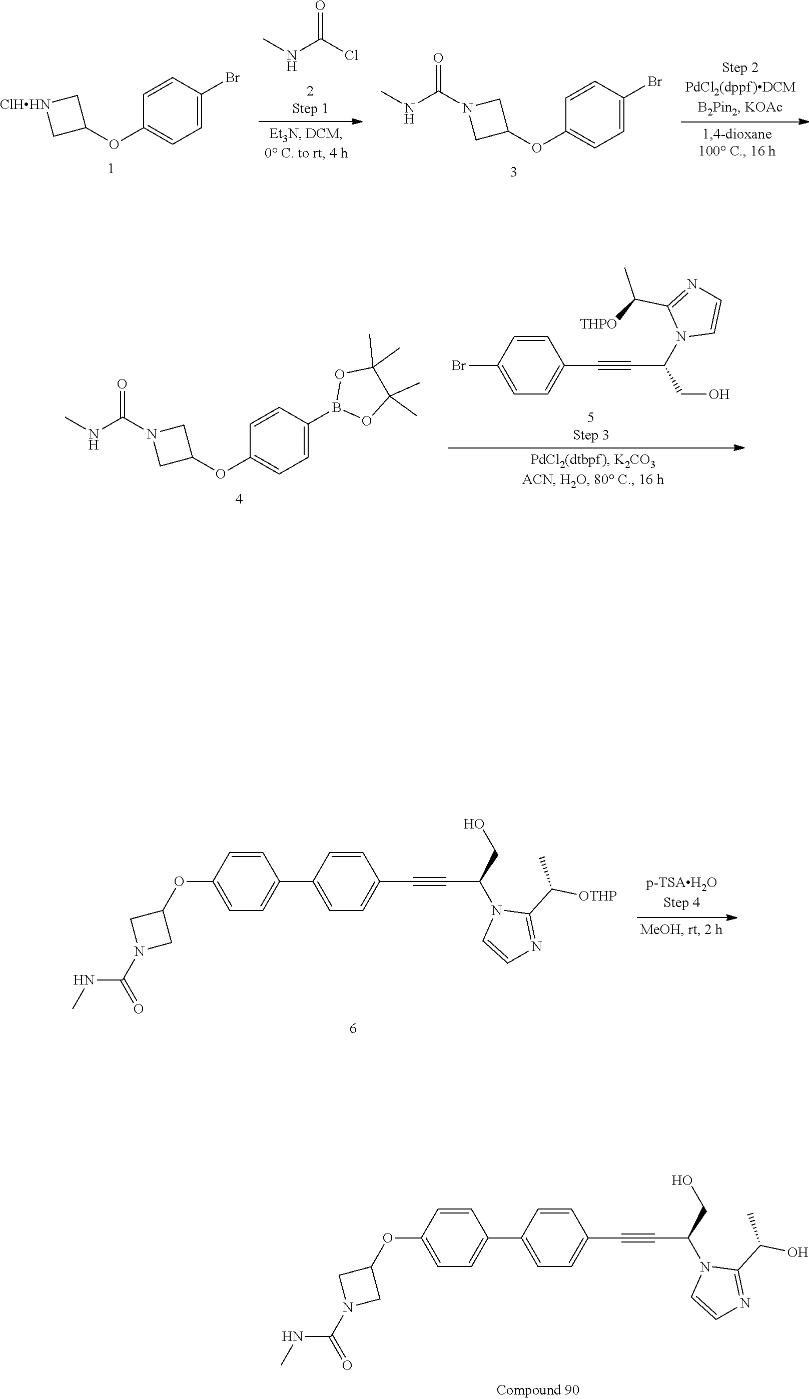

### Step-1:

To a stirred solution of 1 (5.2 g, 22.80 mmol) in DCM (60 mL), was added triethylamine (6.24 mL, 45.6 mmol) at 0° C. and stirred for 15 min. To this reaction mixture, N-Methylcarbamoyl Chloride (2, 2.56 g, 27.4 mmol) was added at 0° C. stirred at room temperature for 4 h. The reaction mixture was quenched with water (20 mL) and extracted with DCM (50 mL×2). The combined organic layer was dried over anhydrous Na₂SO₄, filtered and concentrated under reduced pressure. The resulting crude residue was purified by MPLC (using manually packed SiO₂ cartridge 230-400 mesh size; 5% MeOH in DCM) to obtain 3 as an off-white solid. Yield=6.3 g (88%)

### Step-2:

To a stirred solution of the 3 (3 g, 10.52 mmol) in 1,4-dioxane (30 mL), were added potassium acetate (1.87 g, 31.6 mmol) and bis(pinacolato)diboron (4 g, 15.78 mmol) at room temperature. The resulting mixture was purged with nitrogen for 10 min. To this reaction mixture, PdCl₂(dppf) DCM complex (0.43 g, 0.526 mmol) was added and stirred at 100° C. for 16 h. The reaction mixture was cooled to room temperature, filtered through Celite pad and washed with EtOAc (100 mL). The filtrate was washed with water (100 mL). The organic layer was dried over anhydrous Na₂SO₄, filtered and concentrated under reduced pressure. The crude residue was purified by MPLC (manually packed SiO₂ cartridge 230-400 mesh size; 70% EtOAc in hexanes) to obtain 4 as an off-white solid. Yield=3 g (77%)

### Step-3:

To a solution of 5 (1.5 g, 3.58 mmol) in acetonitrile (15 mL) and water (15 mL), were added 4 (1.783 g, 5.37 mmol) and K₂CO₃ (1.483 g, 10.73 mmol) at room temperature. The resulting mixture was degassed with nitrogen for 10 min. To this reaction mixture, PdCl₂(dtbpf) (0.233 g, 0.358 mmol) was added and stirred at 80° C. for 16 h. The reaction mixture was then diluted with water (30 mL) and extracted with 10% MeOH in DCM (100 mL×2). The combined organic layer was washed with brine (10 mL), dried over anhydrous Na₂SO₄, filtered and concentrated under reduced pressure. The resulting crude residue was purified by MPLC (using manually packed SiO₂ cartridge 230-400 mesh size; 5% MeOH in DCM) to obtain 6 as pale brown solid. Yield: 740 mg (36%). LCMS: Calculated for C31H36N4O5 is 544.652; Observed: 545.2 [M+1]⁺

### Step-4:

To a stirred solution of 6 (0.7 g, 1.28 mmol) in MeOH (10 mL), was added p-toluenesulfonic acid monohydrate (0.73 g, 3.86 mmol) at 0° C. and the reaction mixture stirred at room temperature for 2 h. The volatiles were removed under reduced pressure. The resulting residue was basified with 10% NaHCO3 solution (10 mL) and extracted with 10% MeOH in DCM (100 mL×2). The combined organic extract was dried over anhydrous Na₂SO₄, filtered and concentrated under reduced pressure. The resulting crude residue was purified by MPLC (manually packed SiO₂ cartridge 230-400 mesh size; 5% MeOH in DCM) to afford Compound 90 as an off-white solid. Yield: 150 mg (25%). LCMS: Calculated for C26H28N4O4 is 460.534; Observed: 461.3 [M+1]⁺. 1H-NMR (400 MHz, DMSO-d6): δ 7.67-7.64 (m, 4H), 7.52 (d, J=8.40 Hz, 2H), 7.36 (app d, J=1.20 Hz, 1H), 6.94 (d, J=8.80 Hz, 2H), 6.84 (app d, J=0.80 Hz, 1H), 6.35 (q, J=4.40 Hz, 1H, exchanges with D2O), 5.66 (t, J=6.00 Hz, 1H), 5.53 (t, 1H, exchanges with D2O), 5.37 (d, J=5.60 Hz, 1H, exchanges with D2O), 5.05-5.01 (m, 1H), 4.96-4.91 (m, 1H), 4.27-4.23 (m, 2H), 3.87 (t, J=6.00 Hz, 2H), 3.76-3.73 (m, 2H), 2.55 (d, J=4.40 Hz, 3H), 1.51 (d, J=6.40 Hz, 3H). Product was single isomer with SFC purity=98.7% (LUX-I-Amylose3_0.5% IPAm in MeOH; t_{R}=4.29 min)

### Example A41: Synthesis of Compound 91

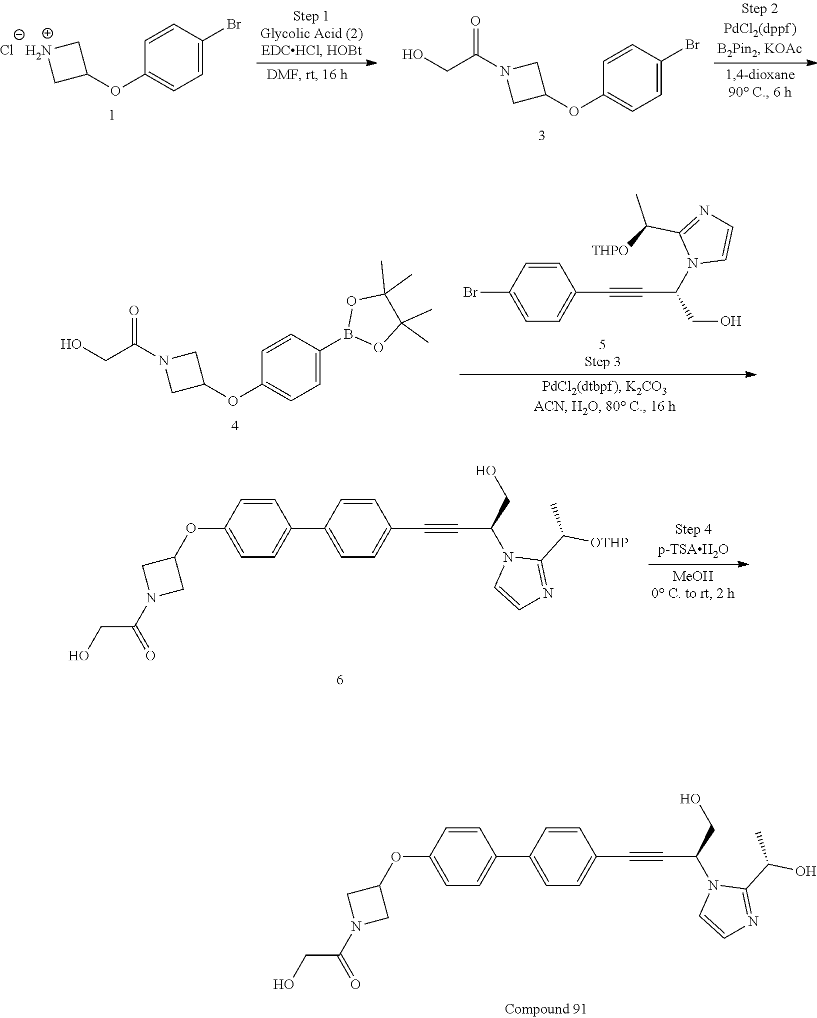

### Step 1:

To a stirred solution of 1 (3 g, 11.34 mmol) and Glycolic Acid (2, 1.035 g, 13.61 mmol) in DMF (50 mL), were added HOBt (2.60 g, 17.01 mmol) and EDC·HCl (3.26 g, 17.01 mmol) at room temperature under nitrogen atmosphere. The reaction was stirred at room temperature for 16 h. The reaction was quenched with ice-cold water and extracted with 5% MeOH in DCM (2×100 mL). The combined organic layer was washed with water (100 mL), brine (100 mL), dried over sodium sulfate, filtered and concentrated under reduced pressure. The crude residue thus obtained was purified by MPLC (using manually packed SiO₂ cartridge, 100-200 mesh; 3% MeOH in DCM) to obtain 3 as an off-white solid. Yield: 2.1 g (58%). LCMS: Calculated for C₁₁H₁₂BrNO₃ is 286.13, Observed: [M]⁺=286.1 and [M+2]⁺=288.1

### Step 2:

To a stirred solution of 3 (2 g, 6.99 mmol) and bis(pinacolato)diboron (2.66 g, 10.48 mmol) in 1,4-dioxane (40 mL), was added potassium acetate (2.058 g, 20.97 mmol). The reaction mixture was purged for 5 min by using nitrogen gas, then added PdCl2(dppf) (0.256 g, 0.349 mmol) and the purging continued for 2 min. The reaction mixture was stirred at 90° C. for 6 h. The reaction mixture was filtered through the Celite pad and washed with EtOAc (100 mL). The combined filtrate was concentrated under reduced pressure. The crude residue thus obtained was purified by MPLC (using manually packed SiO₂ cartridge, 230-400 mesh; 100% EtOAc) to obtain 4 as an off-white solid. Yield: 1.5 g (57%). LCMS: Calculated for C₁₇H₂₄BNO₅ is 333.19, Observed: [M+H]⁺=334.2.

### Step 3:

To a stirred solution of 4 (0.596 g, 1.789 mmol) and 5 (0.5 g, 1.192 mmol) in acetonitrile (6 mL) and water (6 mL), was added K2CO3 (0.494 g, 3.58 mmol). The reaction mixture was purged for 5 min by using nitrogen gas, following which PdCl2(dtbpf) (0.039 g, 0.060 mmol) was added and the purging continued for 2 min. The reaction mixture was stirred at 80° C. for 16 h. The reaction was quenched with water (50 mL) and extracted with 10% MeOH in DCM (2×50 mL). The combined organic layer was washed with water (50 mL), brine (50 mL), dried over anhydrous sodium sulfate, filtered and concentrated under reduced pressure. The crude residue thus obtained was purified by MPLC (using manually packed cartridge; SiO₂ 100-200 mesh; 4% MeOH in DCM) to obtain 6 as a pale-brown gum. Yield: 250 mg (37%). LCMS: Calculated for C₃₁H₃₅N₃O₆ is 545.63, Observed: 546.2 [M+H]⁺

### Step 4:

To a stirred solution of 6 (160 mg, 0.202 mmol) in Methanol (5 mL), was added p-toluenesulfonic acid monohydrate (115 mg, 0.607 mmol) at 0° C. The reaction mixture was stirred at room temperature for 2 h. The reaction was quenched with saturated NaHCO3 solution (20 mL) and extracted with 10% MeOH in DCM (2×30 mL). The combined organic layer was washed with water (30 mL), brine (30 mL), dried over sodium sulfate, filtered and concentrated under reduced pressure. The crude residue thus obtained was purified by MPLC (using manually packed cartridge; SiO₂ 100-200 mesh; 6% MeOH in DCM) to obtain Compound 91 as an off-white solid. Yield: 40 mg (41%). LCMS: Calculated for C₂₆H₂₇N₃O₅ is 461.51, Observed: 462.4 [M+H]⁺. 1H-NMR (400 MHz, DMSO-d6): δ 7.67-7.65 (m, 4H), 7.52 (d, J=8.40 Hz, 2H), 7.37 (s, 1H), 6.95 (d, J=8.80 Hz, 2H), 6.86 (s, 1H), 5.70 (t, J=6.00 Hz, 1H), 5.54 (t, J=5.60 Hz, 1H, exchanges with D2O), 5.40 (d, J=5.60 Hz, 1H, exchanges with D2O), 5.12 (m, 1H), 5.02-5.00 (m, 1H, exchanges with D2O), 4.99-4.97 (m, 1H), 4.67-4.64 (m, 1H), 4.40-4.35 (m, 1H), 4.17-4.15 (m, 1H), 3.95 (d, J=6.00 Hz, 2H), 3.89-3.84 (m, 3H), 1.51 (d, J=6.80 Hz, 3H). SFC purity=96.3% (Column: Whelk-(R,R); Eluents: 0.50% isopropylamine in MeOH and CO2); tR=4.20 min. SFC purity=96.3%

### Example A42: Synthesis of Compound 92

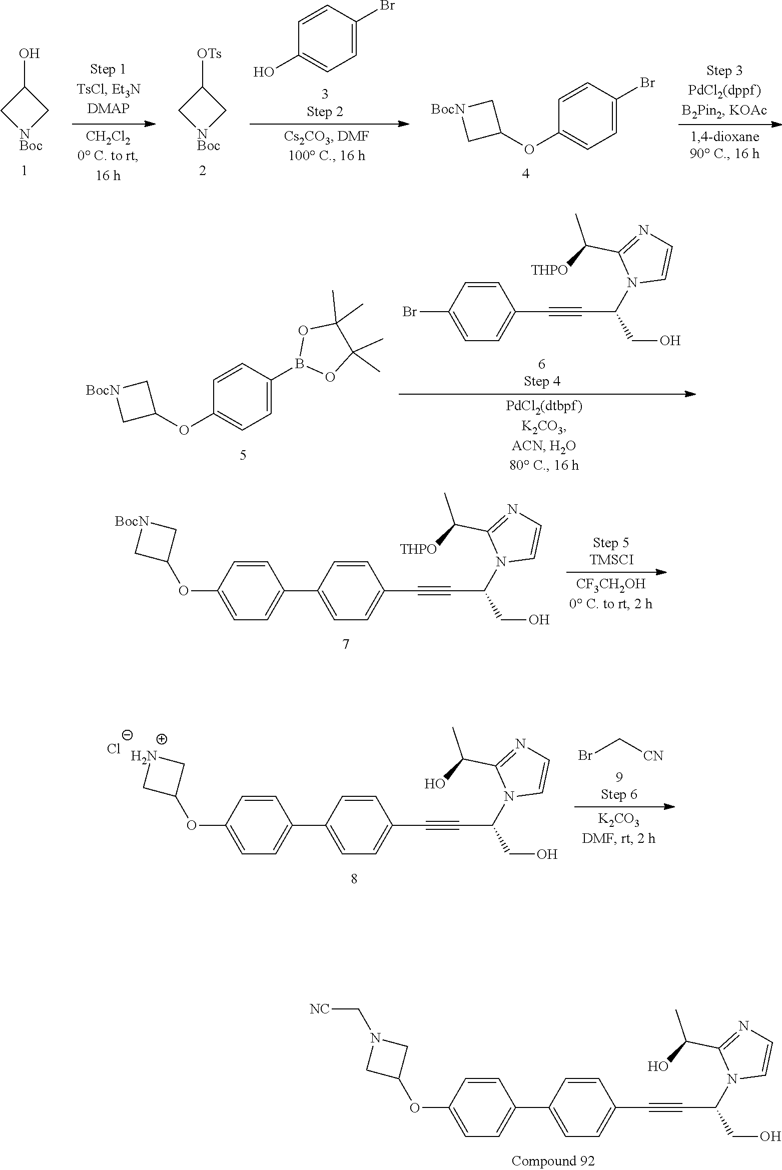

### Step 1:

To a stirred solution of tert-butyl 3-hydroxyazetidine-1-carboxylate (1, 24 g, 139 mmol) in DCM (240 mL), were added Et3N (57.9 mL, 416 mmol), DMAP (1.693 g, 13.86 mmol) and 4-methylbenzenesulfonyl chloride (34.3 g, 180 mmol) at 0° C. The reaction mixture was stirred at room temperature for 16 h. The reaction was quenched with sodium bicarbonate solution (100 mL) and extracted with DCM (200 mL×2). The combined organic extract was washed with brine solution (50 mL), dried over anhydrous Na₂SO₄, filtered and concentrated under reduced pressure. The crude residue was purified by MPLC (manually packed SiO2 cartridge, 100-200 mesh size; 15% EtOAc in hexanes) to give 2 as a yellow liquid. Yield: 40.1 g (87%). LC-MS: Calculated for C15H21NO5S is 327.4, Observed: 228.0 [M-Boc]⁺

### Step 2:

To a solution of 4-bromophenol (3, 16.65 g, 96 mmol) in DMF (250 mL), were added caesium carbonate (41.8 g, 128 mmol) and 2 (21 g, 64.1 mmol) at room temperature. The reaction mixture was stirred at 100° C. for 16 h. The reaction was cooled to room temperature, quenched with ice water (150 mL) and extracted with EtOAc (3×150 mL). The combined organic extract was washed with brine (50 mL), dried over anhydrous Na₂SO₄, filtered and concentrated under reduced pressure. The obtained crude residue was purified by MPLC (manually packed SiO2 cartridge, 100-200 mesh size; 10% EtOAc in hexanes) to afford 4 as an off-white solid. Yield: 19.2 g (90%). LC-MS: Calculated for C14H18BrNO3 is 328.21, Observed: 228.0 [M-Boc] and 230.0 [M-Boc+2]⁺

### Step 3:

To a stirred solution of 4 (12.50 g, 38.1 mmol) in 1,4-dioxane (120 mL), were added potassium acetate (7.48 g, 76 mmol) and bis(pinacolato)diboron (14.51 g, 57.1 mmol) at room temperature. The resulting reaction mixture was purged with nitrogen for 10 min. To this reaction mixture, PdCl2(dppf) (1.393 g, 1.904 mmol) was added and the reaction mixture heated to 90° C. and stirred for 16 h. The reaction mixture was cooled to ambient temperature, filtered through Celite bed. The Celite bed was washed with EtOAc (300 mL) and the filtrate was combined and concentrated under reduced pressure. The resulting crude residue was purified by MPLC (manually packed cartridge; SiO2 230-400 mesh; 5% EtOAc in hexanes) to afford boronate 5 as an off-white solid. Yield: 7.1 g (48%). LC-MS: Calculated for C₂₀H₃₀BNO₅ is 375.2, Observed: 320.2 [M-t-Bu+1]⁺

### Step 4:

To a stirred solution of 6 (2 g, 4.77 mmol) in acetonitrile (25 mL) and water (25 mL), were added boronate 5 (2.68 g, 7.15 mmol) and K2CO3 (1.978 g, 14.31 mmol) at room temperature. The reaction mixture was purged with nitrogen for 5 min. To this reaction mixture, PdCl2(dtbpf) (0.311 g, 0.477 mmol) was added and the reaction mixture heated to 80° C. and stirred for 16 h. One more batch was carried out using 2 g of 6. Both batches were mixed for work-up and purification. The reaction mixture was cooled to ambient temperature. The inorganic solids were filtered through a Celite bed; the Celite bed was washed with 10% MeOH in DCM (100 mL). The combined filtrate was concentrated under reduced pressure. The resulting crude residue was purified by MPLC (manually packed cartridge; SiO₂, 230-400 mesh size; 10% MeOH in DCM) to afford to afford 7 as a brown liquid. Yield=2.3 g (combined yield for both batches). LC-MS: Calculated for C34H41N3O6 is 587.3, Observed: 588.3 [M+1]⁺

### Step 5:

To a stirred solution of 7 (2.3 g, 3.91 mmol) in 2,2,2-trifluoroethanol (30 mL), was added chlorotrimethylsilane (0.750 mL, 5.87 mmol) in a dropwise manner at 0° C. and the reaction mixture stirred at room temperature for 2 h. The volatiles were removed under reduced pressure. The resulting crude residue was triturated with EtOAc (3×10 mL). The obtained solid was dried under reduced pressure to afford 8 as brown solid. Yield=1.7 g (52%). LC-MS: Calculated C24H26N3O3⁺ is 404.49, Observed: 404.2 [M]⁺

### Step 6:

To a solution of 8 (1.7 g, 3.86 mmol) in DMF (20 mL), were added triethylamine (2.69 mL, 19.32 mmol) and 2-bromoacetonitrile (9, 0.404 mL, 5.80 mmol) at room temperature. The resulting reaction mixture was stirred at room temperature for 2 h. The reaction mixture was quenched with water (20 mL) and extracted with 10% MeOH in DCM (3×50 mL). The combined organic extract was washed with brine solution (30 mL), dried over sodium sulfate, filtered, and concentrated under reduced pressure. The resulting crude residue was triturated with MTBE (10 mL×3) to afford 1.59 g of crude residue as a brown gum.

1.2 g of crude residue was purified by MPLC (manually packed cartridge: SiO2, 230-400 mesh size; 6% MeOH in DCM) to afford 400 mg of Compound 92 as an off-white solid. 300 mg of crude residue was purified by preparative HPLC (Column: X-SELECT C18 (250*19 mm); Eluents: (0.1% ammonium bicarbonate in water and acetonitrile) to afford 52 mg of Compound 92 as an off-white solid. Yield: 452 mg. LC-MS: Calculated C26H26N4O3 is 442.20, Observed: 443.2 [M+1]⁺. ¹H-NMR (400 MHz, DMSO-d6): δ 7.67-7.63 (m, 4H), 7.52 (d, J=8.40 Hz, 2H), 7.35 (s, 1H), 6.95 (d, J=8.80 Hz, 2H), 6.84 (s, 1H), 5.69 (t, J=6.00 Hz, 1H), 5.54 (br s, 11H, exchanges with D2O), 5.37 (d, J=5.20 Hz, 1H, exchanges with D2O), 4.95-4.87 (m, 2H), 3.87-3.84 (m, 2H), 3.83-3.80 (m, 2H), 3.75 (s, 2H), 3.26-3.23 (m, 2H), 1.51 (d, J=6.40 Hz, 3H). Single isomer with SFC purity=97.8%

### Example A43: Synthesis of Compound 93

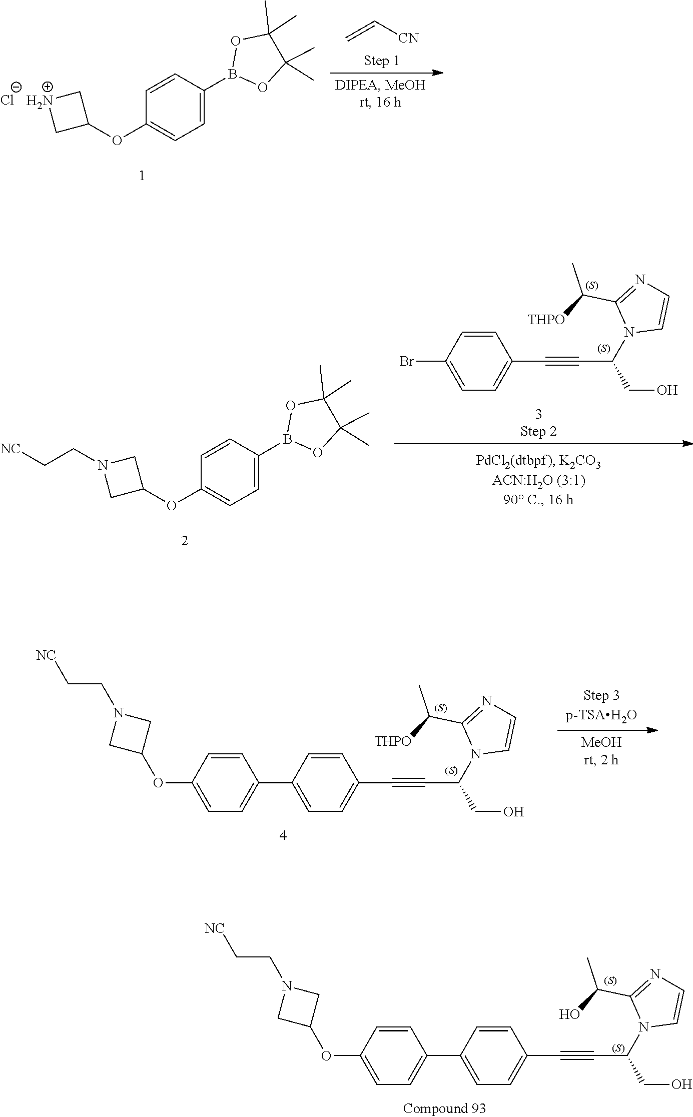

### Step 1:

To a stirred solution of 1 (650 mg, 2.086 mmol) in MeOH (12 mL), were added DIPEA (1.457 mL, 8.34 mmol) and acrylonitrile (0.165 mL, 2.503 mmol) at 0° C. and the resulting reaction mixture stirred at ambient temperature for 16 h. The reaction mixture was quenched with water (30 mL) and extracted with EtOAc (3×45 mL). The combined organic extract was washed with brine solution (50 mL), dried over anhydrous Na2SO4, filtered, and concentrated under reduced pressure. One more reaction was carried out using 50 mg of 1 to get 65 mg of crude compound. Both batches were mixed and purified by using MPLC (manually packed SiO2 cartridge, 230-400 mesh size; 40% EtOAc in hexanes) to afford 2 as a colorless liquid. Yield: 680 mg (two batches). LCMS: Calculated for C18H25BN2O3 is 328.2, Observed: 329.3 [M+1]⁺

### Step 2:

To a stirred solution of 2 (470 mg, 1.431 mmol) in acetonitrile (10 mL) and water (3.33 mL), were added 3 (500 mg, 1.192 mmol) and potassium carbonate (412 mg, 2.98 mmol) at room temperature. The reaction mixture was purged with nitrogen for 5 min. To this reaction mixture, PdCl2(dtbpf) (54.3 mg, 0.083 mmol) was added at room temperature. The resulting reaction mixture was stirred at 90° C. for 16 h. The reaction mixture was quenched with water (50 mL) and extracted with EtOAc (3×90 mL). The combined organic extract was washed with brine (50 mL), dried over Na2SO4, filtered, and concentrated under reduced pressure. The crude residue, thus obtained, was purified by using MPLC (manually packed SiO2 cartridge, 230-400 mesh size; 5% MeOH in DCM) to afford 4 as a brown solid. Yield: 310 mg (46%). LCMS: Calculated for C32H36N4O4 is 540.66, Observed: 540.8 [M+1]⁺

### Step 3:

To a stirred solution of 4 (310 mg, 0.573 mmol) in MeOH (10 mL), was added p-toluenesulfonic acid monohydrate (327 mg, 1.720 mmol) at 0° C. and the resulting reaction mixture stirred at room temperature for 2 h. The reaction mixture was quenched with saturated NaHCO3 solution (20 mL) and extracted with 10% MeOH in DCM (3×50 mL). The combined organic layer was washed with brine (50 mL), dried over Na2SO4, filtered, and concentrated under reduced pressure. The crude residue, thus obtained, was purified by preparative HPLC by using (Column: X-SELECT C18 (250*19 mm); Eluents: 0.1% ammonium bicarbonate in water and acetonitrile) to afford Compound 93 as an off-white solid. Yield: 66 mg (25%). LCMS: Calculated for C27H28N4O3 is 456.55, Observed: 457.3 [M+H]⁺. 1H-NMR (400 MHz, DMSO-d6): δ 7.66-7.62 (m, 4H), 7.51 (d, J=8.40 Hz, 2H), 7.35 (app d, J=0.80 Hz, 1H), 6.94 (d, J=8.80 Hz, 2H), 6.84 (app d, J=0.80 Hz, 1H), 5.67 (t, 1H), 5.53 (t, J=5.60 Hz, 1H, 1H, exchanges with D2O), 5.37 (d, J=5.60 Hz, 1H, 1H, exchanges with D2O), 4.95-4.92 (m, 1H), 4.88-4.81 (m, 1H), 3.87 (t, J=6.00 Hz, 2H), 3.83-3.79 (m, 2H), 3.10-3.07 (m, 2H), 2.69 (t, J=6.40 Hz, 2H), 2.56-2.51 (m, 2H), 1.51 (d, J=6.40 Hz, 3H). SFC: 98.6%; tR=3.03 min (Column: Whelk-(R,R); Eluents: 0.5% isopropyl amine in MeOH); LUX-l-Amylose3_0.5% IPAm in MeOH tR=8.80 min. SFC purity=98.6%

### Example A44: Synthesis of Compound 94

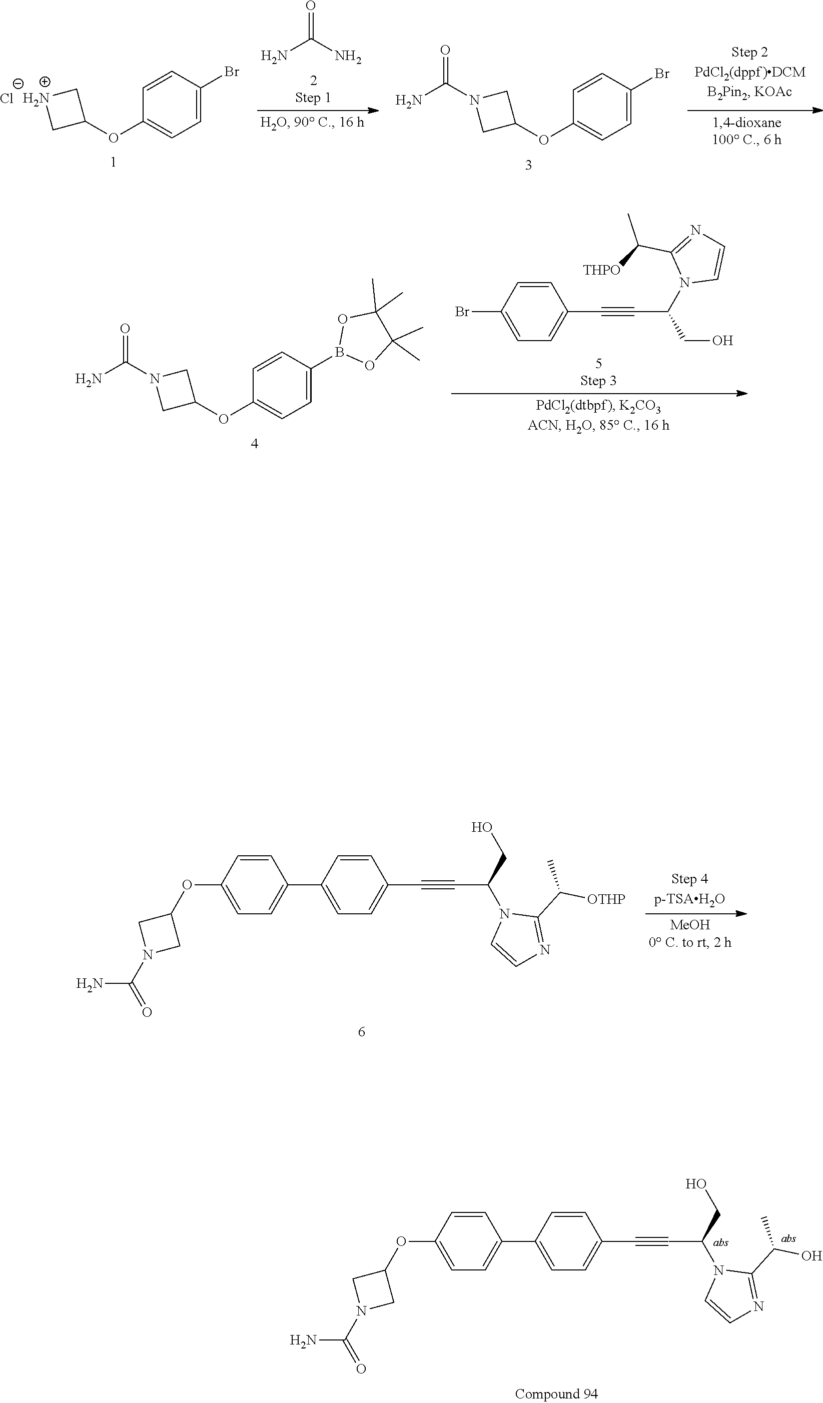

### Step-1:

To a stirred solution of 1 (2 g, 7.56 mmol) in water (50 mL), was added urea (2, 0.681 g, 11.34 mmol) at room temperature, and the reaction mixture stirred at 90° C. for 16 h. The obtained solid was filtered and dried under vacuum to get 3 as an off-white solid. Yield: 1.2 g (49%). LCMS: Calculated for C₁₀H₁₁BrN₂O₂ is 271.11; Observed: 270.8 [M]⁺ and 273.0 [M+2]⁺

### Step 2:

To a stirred solution of 3 (1.2 g, 4.43 mmol) in 1,4-dioxane (30 mL), were added potassium acetate (1.303 g, 13.28 mmol) and bis(pinacolato)diboron (1.686 g, 6.64 mmol). After bubbling nitrogen through the reaction mixture for 5 min, PdCl₂(dppf)·DCM (0.162 g, 0.221 mmol) was added and the bubbling of nitrogen continued for 2 min. The reaction mixture was stirred at 100° C. for 6 h. The reaction mixture was filtered through the celite pad and washed with EtOAc (100 mL). The combined filtrate was concentrated under reduced pressure. The crude residue was purified by MPLC (using manually packed cartridge; SiO₂ 100-200 mesh; 3% MeOH in DCM) to obtain 4 as a pale-brown solid. Yield: 1 g (63%). LCMS: Calculated for C₁₆H₂₃BN₂O₄ is 318.18; Observed: 319.0 [M+1]⁺

### Step 3:

To a stirred solution of 4 (626 mg, 1.967 mmol) and 5 (550 mg, 1.312 mmol) in a mixture of acetonitrile (6 mL) and water (6 mL), was added K₂CO₃ (544 mg, 3.93 mmol). After bubbling nitrogen through the reaction mixture for 5 min, PdCl2(dtbpf) (42.7 mg, 0.066 mmol) was added and the bubbling of nitrogen continued for 2 min. The reaction mixture was stirred at 80° C. for 16 h. The reaction mixture was quenched with water (50 mL) and extracted with 10% MeOH in DCM (2×50 mL). The combined organic layer was washed with water (50 mL), brine (50 mL), dried over sodium sulfate, filtered and concentrated under reduced pressure. The crude residue, thus obtained, was purified by MPLC (using manually packed cartridge; SiO₂ 100-200 mesh; 5% MeOH in DCM) to obtain 6 as a pale brown gum. Yield: 290 mg (40%). LCMS: Calculated for C₃₀H₃₄N₄O₅ is 530.62; Observed: 531.3 [M+1]⁺

### Step 4:

To a stirred solution of 6 (290 mg, 0.547 mmol) in MeOH (6 mL), was added p-toluenesulfonic acid monohydrate (312 mg, 1.640 mmol) at 0° C. and the reaction mixture stirred at room temperature for 2 h. The reaction was quenched with saturated NaHCO₃ solution (10 mL) and extracted with 10% MeOH in DCM (2×50 mL). The combined organic layer was washed with water (50 mL), brine (50 mL), dried over sodium sulfate, filtered and the filtrate concentrated under reduced pressure. The crude residue was purified by reverse phase column chromatography (Column: Redisep Gold, C-18 SiO2; Eluents: 10 mM ammonium bicarbonate in water and ACN) to obtain Compound 94 as an off-white solid. Yield: 27 mg (10%). LCMS: Calculated for C₂₅H₂₆N₄O₄ is 446.51; Observed: 447.0 [M+1]⁺. 1H-NMR (400 MHz, DMSO-d6): S 7.67-7.65 (m, 4H), 7.52 (d, J=8.40 Hz, 2H), 7.36 (d, J=1.20 Hz, 1H), 6.94 (d, J=8.80 Hz, 2H), 6.84 (d, J=0.80 Hz, 1H), 6.00 (br s, 2H, exchanges with D2O), 5.69 (t, J=6.00 Hz, 1H), 5.53 (t, J=5.60 Hz, 1H, exchanges with D2O), 5.37 (d, J=5.60 Hz, 1H, exchanges with D2O), 5.05-5.02 (m, 1H), 4.96-4.93 (m, 1H), 4.27-4.23 (m, 2H), 3.87 (t, J=5.60 Hz, 2H), 3.76-3.73 (m, 2H), 1.51 (d, J=6.40 Hz, 3H). SFC: 94.0%; tR=3.57 min (Column: R,R-Whelk; Eluents: CO2 and 0.5% isopropyl amine in MeOH). SFC purity=94.0%

### Example A45: Synthesis of Compound 95

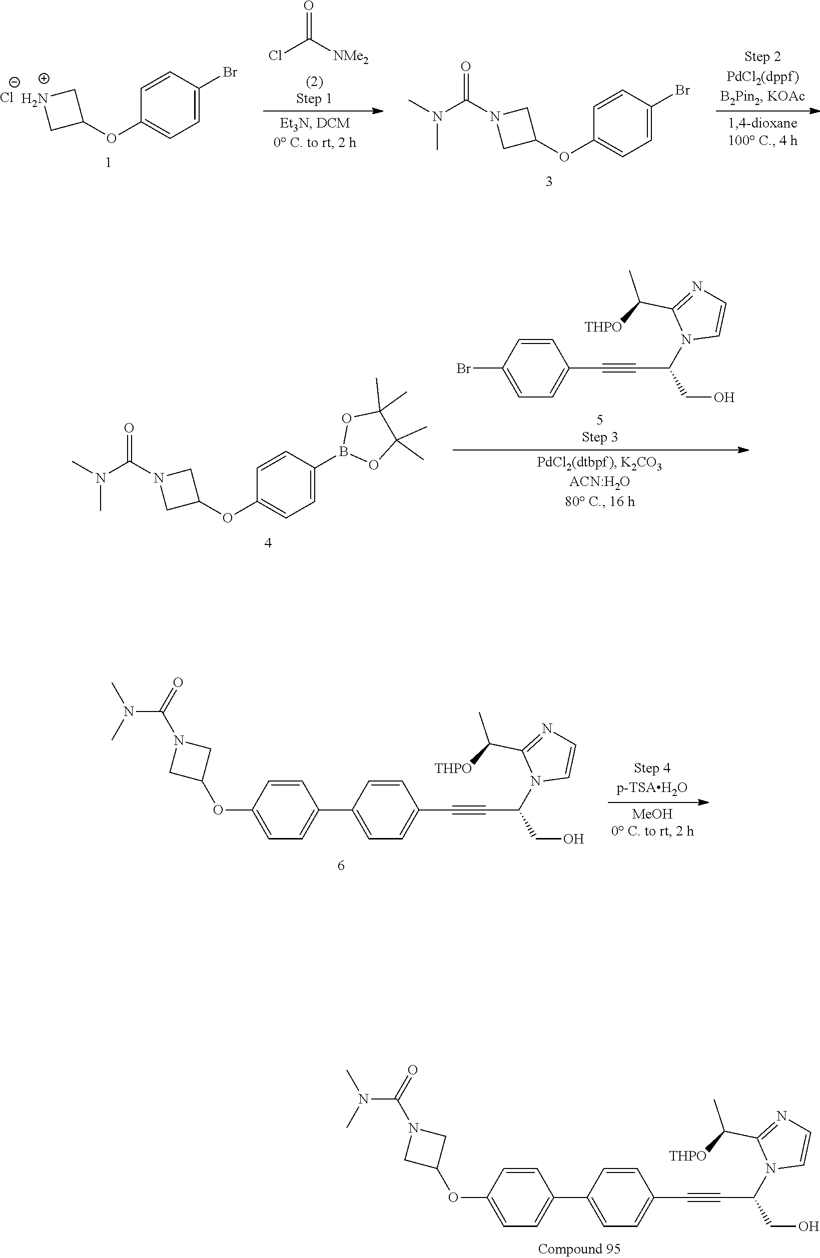

### Step-1:

To a stirred solution of 1 (2 g, 7.56 mmol) in DCM (20 mL), was added triethylamine (3.16 mL, 22.68 mmol) at 0° C. and stirred for 15 min. To this reaction mixture, Dimethylcarbamoyl chloride (2, 0.84 mL, 9.07 mmol) was added at 0° C. and the reaction mixture stirred at room temperature for 2 h. The reaction mixture was quenched with 10% NaHCO3 solution (20 mL) and extracted with DCM (40 mL×2). The combined organic layer was dried over anhydrous Na₂SO₄, filtered, and concentrated under reduced pressure. The resulting crude residue was purified by using MPLC (manually packed SiO₂ cartridge, 100-200 mesh size; 10% EtOAc in hexanes) to obtain 3 as an off-white solid with 73% purity by LC-MS; The product was taken to the next step without further purification. Yield=1.5 g (48%). LCMS: Calculated for C12H15BrN2O2 is 299.17; Observed: 299.1 [M]⁺ and 301.1 [M+2]⁺

### Step-2:

To a stirred solution of the 3 (1.5 g, 5.01 mmol) in dioxane (25 mL), were added potassium acetate (1.78 g, 7.02 mmol) and bis(pinacolato)diboron (1.48 g, 15.04 mmol) at room temperature, and a stream of nitrogen gas bubbled through the reaction mixture for 5 min. To this reaction mixture, PdCl₂(dppf) (0.18 g, 0.25 mmol) was added and stirred at 100° C. for 4 h. The reaction mixture was cooled to room temperature, filtered through Celite pad, and washed with EtOAc (60 mL). The filtrate was washed with water (30 mL). The organic layer was dried over anhydrous Na₂SO₄, filtered, and concentrated under reduced pressure. The crude residue was purified by MPLC (manually packed SiO₂ cartridge, 100-200 mesh size; 50% EtOAc in hexanes) to obtain 4 as an off-white solid. Yield=1.2 g (66%). LCMS: Calculated for C18H27BN2O4 is 346.23; Observed: 347.3 [M+1]⁺

### Step-3:

To a solution of 5 (0.6 g, 1.43 mmol) in acetonitrile (8 mL) and water (8 mL), were added 4 (0.74 g, 2.14 mmol) and K₂CO₃ (0.59 g, 4.29 mmol) at room temperature, and a stream of nitrogen gas bubbled through the reaction mixture for 10 min. To this reaction mixture, PdCl₂(dtbpf) (0.093 g, 0.143 mmol) was added and stirred at 80° C. for 16 h. The reaction mixture was then quenched with water (50 mL) and extracted with 10% MeOH in DCM (50 mL×2). The combined organic layer was washed with brine (50 mL), dried over anhydrous Na₂SO₄, filtered, and concentrated under reduced pressure. The resulting crude residue was purified by using MPLC (manually packed SiO₂ cartridge, 230-400 mesh size; 6% MeOH in DCM) to obtain 6 as pale-brown solid. Yield: 390 mg (46%). LCMS: Calculated for C32H38N4O5 is 558.68; Observed: 559.3 [M+1]⁺

### Step-4:

To a stirred solution of 6 (0.38 g, 0.68 mmol) in MeOH (10 mL), was added p-toluenesulfonic acid monohydrate (0.39 g, 2.04 mmol) at 0° C., and the reaction mixture stirred at room temperature for 2 h. The volatiles were removed under reduced pressure. The resulting residue was dissolved in 10% MeOH in DCM (50 mL), washed with 10% NaHCO3 solution (10 mL). The organic extract was dried over anhydrous Na₂SO₄, filtered, and concentrated under reduced pressure. The resulting crude residue was purified by reverse phase column chromatography (Column: Redisep Gold, C18 reverse phase SiO2; Eluents: 10 mM ammonium bicarbonate in water and ACN) to afford Compound 95 as an off-white solid. Yield: 70 mg (22%) LCMS: Calculated for C27H30N4O4 is 474.56; Observed: 475.3 [M+1]⁺. 1H-NMR (400 MHz, DMSO-d6): δ 7.67-7.63 (m, 4H), 7.52 (dd, J=2.00, 6.60 Hz, 2H), 7.36 (app d, J=1.20 Hz, 1H), 6.94 (d, J=2.00 Hz, 2H), 6.84 (app d, J=1.60 Hz, 1H), 5.68 (t, J=6.00 Hz, 1H), 5.53 (t, J=6.00 Hz, 1H, exchanges with D2O), 5.37 (d, J=5.60 Hz, 1H, exchanges with D2O), 5.06-5.01 (m, 1H), 4.97-4.91 (m, 1H), 4.37-4.33 (m, 2H), 3.88-3.85 (m, 4H), 2.78 (s, 6H), 1.51 (d, J=6.40 Hz, 3H). SFC: 94.9%; tR=4.80 min (Column: Whelk-(R,R); Eluents: CO2 and 0.5% isopropyl amine in MeOH). Product was a single isomer with SFC purity=95%

### Example A46: Synthesis of Compound 96

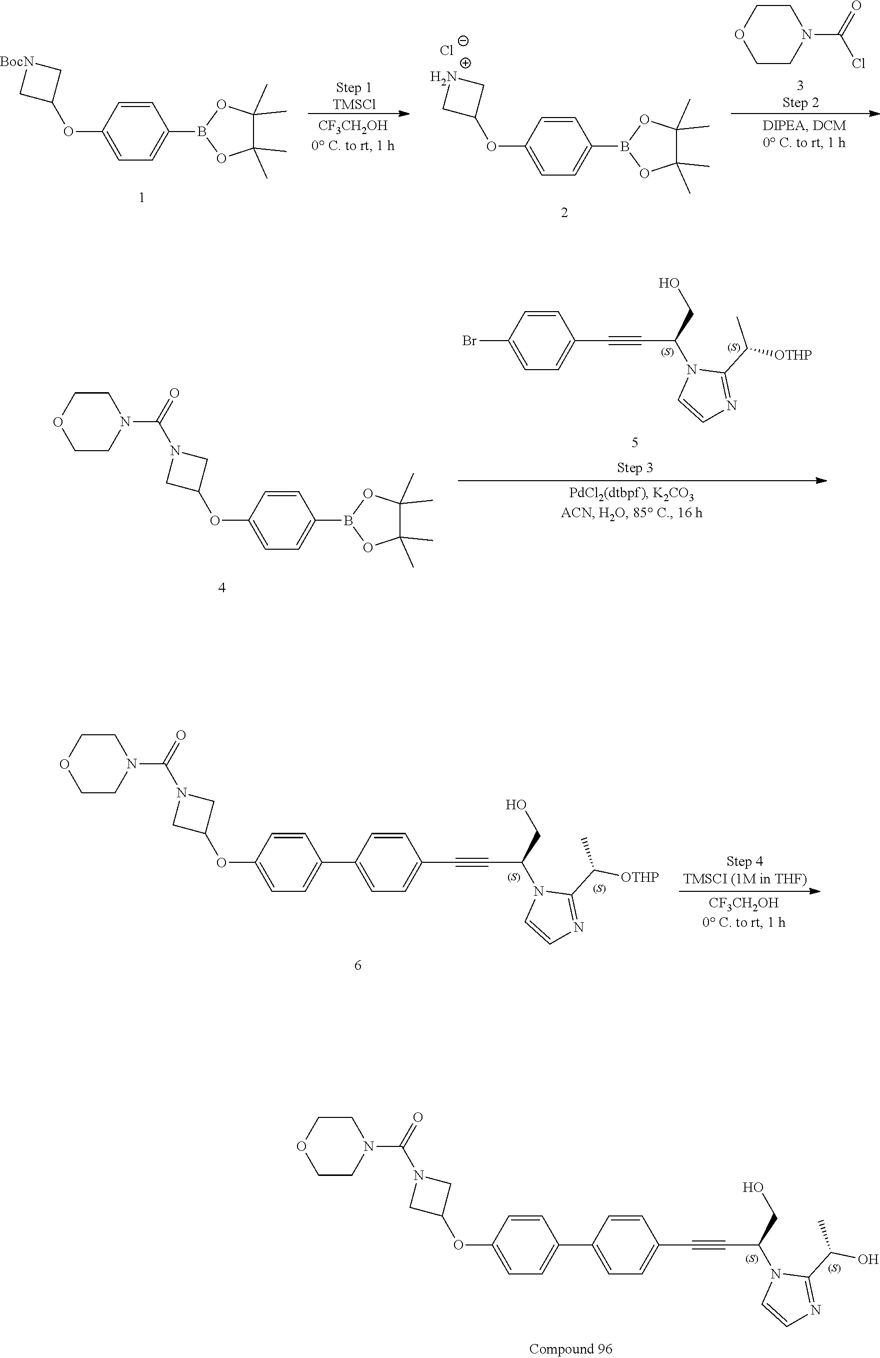

### Step-1:

To a stirred solution of 1 (5.9 g, 15.72 mmol) in 2,2,2-trifluoroethanol (50 mL), was added TMSCl (5.02 mL, 39.3 mmol) at 0° C., and the reaction mixture stirred at room temperature for 1 h. The reaction mixture was concentrated under reduced pressure to afford 2 as a white solid. Crude yield=4.2 g. LC-MS: Calculated C₁₅H₂₃BNO3⁺ is 276.16, Observed: 276.4 [M+1]⁺

### Step-2:

To a solution of 2 (2 g, 7.27 mmol) in DCM (20 mL), were added DIPEA (2.349 g, 18.17 mmol) and morpholine-4-carbonyl chloride (3, 1.305 g, 8.72 mmol) at 0° C. The reaction mixture was stirred at room temperature for 1 h. The reaction was cooled to room temperature, quenched with water (30 mL) and extracted with DCM (3×60 mL). The combined organic extract was washed with brine (20 mL), dried over anhydrous Na₂SO₄, filtered and concentrated under reduced pressure. The resulting crude mass was purified by using MPLC (manually packed SiO2 cartridge; 100-200 mesh size; 62% EtOAc in hexanes) to afford 4 as an off-white solid. Yield: 1.7 g (57%). LC-MS: Calculated C₂₀H₂₉BN₂O₅ is 388.27, Observed: 389.2 [M+1]⁺

### Step-3:

To a stirred solution of 5 (500 mg, 1.192 mmol) in dioxane (8 mL) and water (2 mL), were added 4 (556 mg, 1.431 mmol) and potassium phosphate tribasic (759 mg, 3.58 mmol) at room temperature and the reaction mixture purged with nitrogen for 5 min. To this reaction mixture, PdCl2(dppf) (61.1 mg, 0.083 mmol) was added and the purging continued for another 2 min. The reaction mixture was then stirred at 85° C. for 16 h. The reaction was cooled to ambient temperature, filtered through a Celite bed. The Celite bed was washed with EtOAc (90 mL). The filtrate was combined and then concentrated under reduced pressure. The resulting crude mass, thus obtained, was purified by using MPLC (manually packed SiO₂ cartridge; 230-400 mesh size; 10% MeOH in DCM) to afford 6 as a brown solid. Yield: 510 mg (66%). LC-MS: Calculated C₃₄H₄₀N₄O₆ is 600.71, Observed: 601.3 [M+1]⁺

### Step-4:

To a stirred solution of 6 (0.450 g, 0.749 mmol) in 2,2,2-trifluoroethanol (6 mL), was added TMSCl (1 M in THF, 0.375 mL, 0.375 mmol) at 0° C. The reaction mixture was stirred at room temperature for 1 h. The volatiles in the reaction mixture were removed under reduced pressure. The crude mass, thus obtained, was purified by reversed phase preparative HPLC (Column: X-Bridge C8 (4.6×150 mm), 5 μm; Eluents: 10 mM ammonium bicarbonate in water and ACN) to afford Compound 96 as an off-white solid. Yield=50 mg (13%). LC-MS: Calculated C₂₉H₃₂N₄O₅ is 516.59, Observed: 517.0 [M+1]⁺. 1H-NMR (400 MHz, DMSO-d6): δ 7.67-7.64 (m, 4H), 7.52 (d, J=8.40 Hz, 2H), 7.35 (app d, J=1.20 Hz, 1H), 6.93 (d, J=8.80 Hz, 2H), 6.84 (app d, J=0.80 Hz, 1H), 5.69 (t, J=6.00 Hz, 1H), 5.53 (t, J=5.60 Hz, 1H, ex changes with D2O), 5.37 (d, J=5.60 Hz, 1H, exchanges with D2O), 5.07-5.04 (m, 1H), 4.95-4.92 (m, 1H), 4.39 (t, J=6.40 Hz, 2H), 3.92-3.85 (m, 4H), 3.55-3.53 (m, 4H), 3.24-3.22 (m, 4H), 1.51 (d, J=6.40 Hz, 3H). SFC: 97.4%; tR=4.03 min (Column: I-cellulose B; Eluents: CO2 and 0.5% isopropyl amine in MeOH)

### Example A47: Synthesis of Compound 97

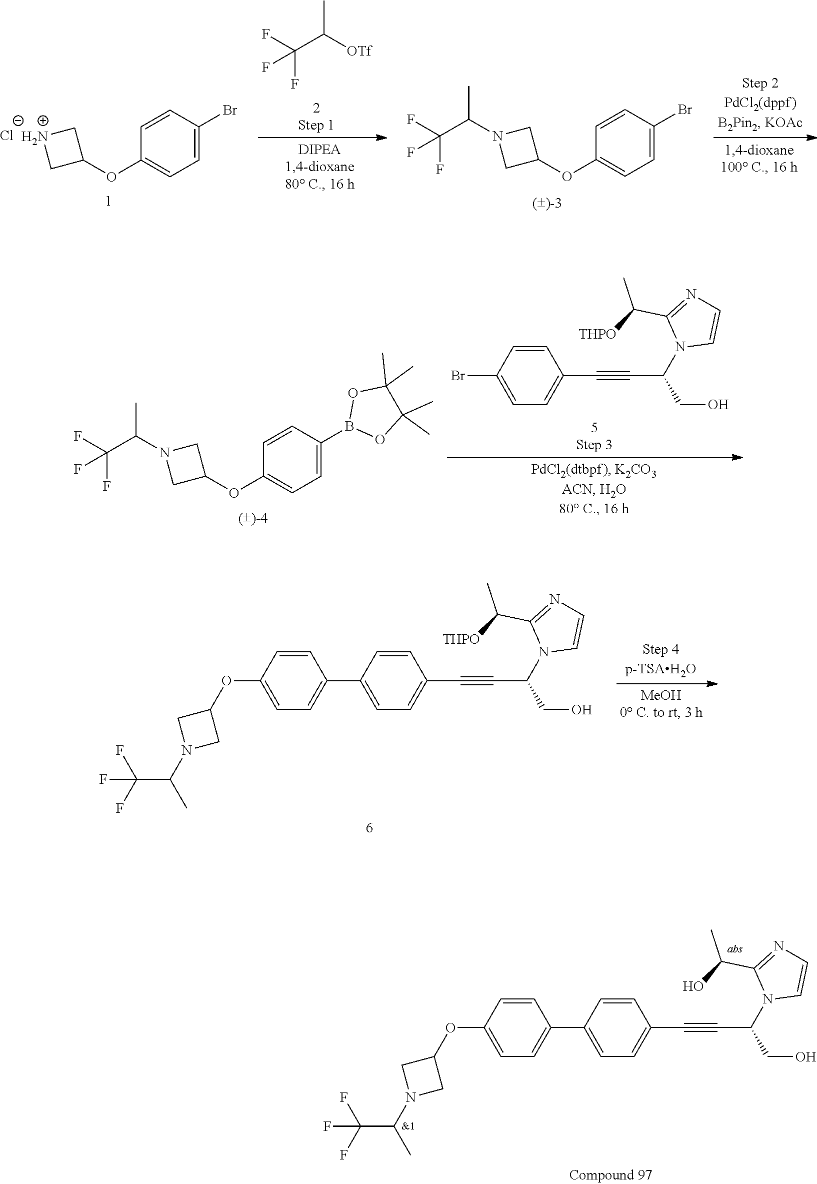

### Step-1:

To a stirred solution of 1 (2 g, 7.56 mmol) in dioxane (20 mL), were added DIPEA (3.95 mL, 22.68 mmol) and 1,1,1-trifluoropropan-2-yl trifluoromethanesulfonate (2, 2.05 g, 8.32 mmol) at room temperature and the resulting reaction mixture stirred at 80° C. for 16 h. The reaction mixture was quenched with water and extracted with EtOAc (50 mL×2). The combined organic layer was dried over anhydrous Na₂SO₄, filtered and concentrated under reduced pressure. The resulting crude residue was purified by MPLC (using manually packed SiO₂ cartridge, 230-400 mesh size; 70% EtOAc in hexanes) to obtain (±)-3 as colorless liquid. Yield=1 g (27%). LCMS: Calculated for C12H13BrF3NO is 324.14; Observed: 324.2 [M]⁺ and 326.2 [M+2]⁺

### Step-2:

To a stirred solution of (±)-3 (1 g, 3.09 mmol) in dioxane (25 mL), were added potassium acetate (0.90 g, 9.26 mmol) and bis(pinacolato)diboron (1.17 g, 4.63 mmol) at room temperature. The resulting mixture was purged with nitrogen for 5 min. To this reaction mixture, PdCl₂(dppf) (0.11 g, 0.15 mmol) was added and stirred at 100° C. for 16 h. The reaction mixture was cooled to room temperature, filtered through Celite pad and washed with EtOAc (200 mL). The filtrate was washed with water (50 mL). The organic layer was dried over anhydrous Na₂SO₄, filtered and concentrated under reduced pressure. The resulting crude residue was purified by MPLC (using manually packed SiO₂ cartridge, 100-200 mesh size; 70% EtOAc in hexanes) to obtain (±)-4 as an off-white solid. Yield=850 mg (63%)

### Step-3:

To a solution of 5 (0.48 g, 1.14 mmol) in acetonitrile (8 mL) and water (8 mL), were added (±)-4 (0.64 g, 1.71 mmol) and K₂CO₃ (0.47 g, 3.43 mmol) at room temperature. The resulting mixture was purged with nitrogen for 10 min. To this reaction mixture, PdCl₂(dtbpf) (0.075 g, 0.11 mmol) was added and stirred at 80° C. for 16 h. The reaction mixture was then quenched with water (50 mL) and extracted with 10% MeOH in DCM (50 mL×2). The combined organic layer was dried over anhydrous Na₂SO₄, filtered and concentrated under reduced pressure. The resulting crude residue was purified by MPLC (using manually packed SiO₂ cartridge, 230-400 mesh size; 5% MeOH in DCM) to obtain 6 as pale-brown solid. Yield: 310 mg (42%). LCMS: Calculated for C32H36F3N3O4 is 583.65; Observed: 584.3 [M+1]⁺

### Step-4:

To a stirred solution of 6 (0.3 g, 0.51 mmol) in MeOH (10 mL), was added p-toluenesulfonic acid monohydrate (0.29 g, 1.54 mmol) at 0° C. and the reaction mixture stirred at room temperature for 3 h. The volatiles were removed under reduced pressure, the resulting residue dissolved in 10% MeOH in DCM (100 mL) and washed with 10% NaHCO3 solution (10 mL). The organic layer was dried over anhydrous Na₂SO₄, filtered and concentrated under reduced pressure to give a crude residue which was purified by reverse phase column chromatography (Column: Redisep C18; Eluents: 10 mM ammonium bicarbonate in water and ACN) to afford Compound 97 as an off-white solid. Yield: 100 mg (39%). LCMS: Calculated for C27H28F3N3O3 is 499.53; Observed: 500.8 [M+1]⁺. 1H-NMR (400 MHz, DMSO-d6): δ 7.66-7.62 (m, 4H), 7.52 (d, J=8.40 Hz, 2H), 7.35 (app d, J=1.20 Hz, 1H), 6.95 (d, J=8.80 Hz, 2H), 6.84 (app d, J=1.20 Hz, 1H), 5.68 (t, J=6.00 Hz, 1H), 5.53 (t, J=5.60 Hz, 1H, exchanges with D2O), 5.37 (d, J=5.60 Hz, 1H, exchanges with D2O), 4.96-4.91 (m, 1H), 4.90-4.87 (m, 1H), 3.88-3.77 (m, 4H), 3.29-3.22 (m, 2H), 3.21-3.16 (m, 1H), 1.51 (d, J=6.40 Hz, 3H), 1.09 (d, J=6.80 Hz, 3H). 19F-NMR (376. MHz, DMSO-d6): δ −74.44. LUX-I-Amylose3_0.5% IPAm in MeOH tR=3.01 min and 3.17 min. Final product was mixture of diastereomers (racemic in the tail part)

### Example A48: Synthesis of Compound 98

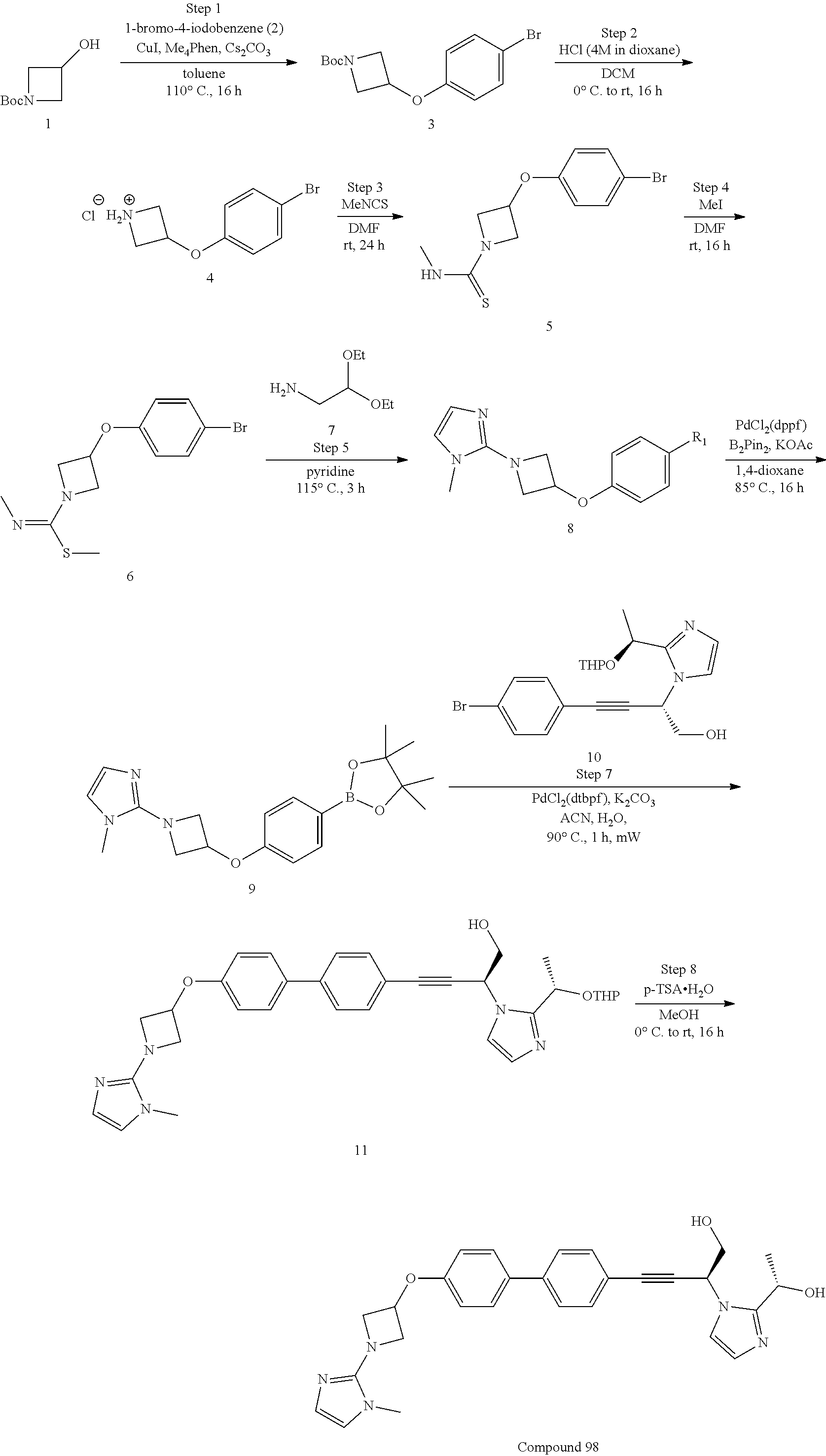

### Step 1:

To a stirred solution of 3,4,7,8-tetramethyl-1,10-phenanthroline (Me4Phen, 1.671 g, 7.07 mmol) and CuI (0.673 g, 3.53 mmol) in toluene (200 mL), was added Cs₂CO₃ (46.1 g, 141 mmol). The resulting mixture was purged with nitrogen for 5 min. Then, 1-bromo-4-iodobenzene (2, 20 g, 70.7 mmol) and tert-butyl 3-hydroxyazetidine-1-carboxylate (1, 13.47 g, 78 mmol) were added and the reaction mixture stirred at 110° C. for 16 h. The reaction mixture was then cooled to room temperature and filtered through a pad of Celite bed. The Celite bed was washed with EtOAc (100 mL). The combined filtrate was concentrated under reduced pressure. The resulting crude residue was purified by using MPLC (manually packed SiO₂ cartridge, 100-200 mesh size; 8-10% EtOAc in hexanes) to get 3 as an off-white solid. Yield: 19.5 g (76%). LC-MS: Calculated for C₁₄H₁₈BrNO₃ is 328.21, Observed: 228.1 [M-Boc]⁺ and 230.1 [(M-Boc)+2]⁺

### Step 2:

To a stirred solution of 3 (19.5 g, 59.4 mmol) in DCM (200 mL), was added 4 M HCl in 1,4-dioxane (29.7 mL, 119 mmol) dropwise at 0° C. and the reaction mixture stirred at room temperature for 16 h. The reaction mixture was then concentrated under reduced pressure to get a crude residue. The residue was triturated with hexanes (500 mL), filtered, and dried to get 4 as a white solid. Yield: 15.6 g (87%). LC-MS: Calculated for C₉H₁₁BrNO⁺ is 228, Observed: 228.2 [M]⁺ and 230.1 [M+2]⁺

### Step 3:

To a stirred solution of 4 (3 g, 13.15 mmol) in DMF (10 mL), was added a solution of Methyl isothiocyanate (1.442 g, 19.73 mmol) in DMF (3 mL) dropwise at room temperature and the reaction mixture stirred at room temperature for 24 h. The reaction mixture was then concentrated under reduced pressure. The resulting crude residue was suspended in ice-cold water (100 mL) and stirred for 30 min. The obtained precipitate was filtered, washed with water (50 mL), and dried to get 5 as an off-white solid. Yield: 3.6 g (84%). LC-MS: Calculated for C₁₁H₁₃BrN₂OS is 301.20, Observed: 301.1 [M]⁺ and 303.0 [M+2]⁺

### Step 4:

To a stirred solution of 5 (3.6 g, 11.95 mmol) in DMF (30 mL), was added a dropwise solution of methyl iodide (1.860 mL, 29.9 mmol) in DMF (10 mL) at 0° C. The reaction mixture was stirred at room temperature for 16 h. The reaction mixture was concentrated under reduced pressure. The resulting crude residue was suspended in ice-cold water (150 mL) and stirred for 1 h. The obtained precipitate was filtered, washed with water (2×70 mL), and dried to get 6 as an off-white solid. Yield: 3.7 g (93%). LC-MS: Calculated for C₁₂H₁₅BrN₂OS is 315.23, Observed: 315.2 [M]⁺ and 317.1 [M+2]⁺

### Step 5:

To a stirred solution 6 (3.7 g, 11.74 mmol) in pyridine (40 mL) was added Aminoacetaldehyde diethyl acetal (1.543 mL, 14.08 mmol) dropwise at room temperature and the reaction mixture stirred at 115° C. for 3 h. The reaction mixture was then concentrated under reduced pressure. To the resulting residue, 2 N HCl (50 mL) was added and heated at 100° C. for 1 h. The reaction mixture was diluted with water (70 mL), basified with saturated NaHCO₃ solution (50 mL) and extracted with EtOAc (2×100 mL). The combined organic layer was dried over anhydrous Na₂SO₄, filtered and concentrated under reduced pressure to get a crude residue which was purified by using MPLC (manually packed SiO₂ cartridge, 100-200 mesh size; 5% MeOH in DCM) to get 8 as an off-white solid. Yield: 1.2 g (29%). LC-MS: Calculated for C₁₃H₁₄BrN₃O is 308.18, Observed: 308.2 [M]⁺ and 310.0 [M+2]⁺

### Step 6:

To a stirred solution of 8 (1 g, 3.24 mmol) in dioxane (15 mL), were added bis(pinacolato)diboron (1.236 g, 4.87 mmol) and potassium acetate (0.637 g, 6.49 mmol) at room temperature and the reaction mixture purged with nitrogen for 10 min. Then, Pd(dppf)Cl₂ (0.237 g, 0.324 mmol) was added and the reaction mixture stirred at 85° C. for 16 h. The reaction mixture was cooled to room temperature and filtered through a pad of Celite. The Celite bed was washed with DCM (200 mL). The combined filtrate was concentrated under reduced pressure. The resulting crude residue was purified by using MPLC (manually packed SiO₂ cartridge, 100-200 mesh size; 4% MeOH in DCM) to get 9 as a brown gum. Yield: 0.9 g (53%). LC-MS: Calculated for C₁₉H₂₆BN₃O₃ is 355.25, Observed: 356.3 [M+1]⁺

### Step 7:

To a stirred solution of 10 (300 mg, 0.715 mmol) and 9 (330 mg, 0.930 mmol) in a mixture of acetonitrile (10 mL) and water (2 mL), was added potassium carbonate (297 mg, 2.146 mmol) at room temperature. The reaction mixture was subjected to microwave irradiation in a Biotage microwave reactor at 90° C. for 1 h. The reaction mixture was diluted with DCM (50 mL) and filtered through a pad of Celite. The Celite bed was washed with DCM (2×80 mL). The combined filtrate was concentrated under reduced pressure. The resulting crude residue was purified by using MPLC (manually packed SiO₂ cartridge, 100-200 mesh size; 6% MeOH in DCM) to get 11 as a brown solid. Yield: 0.25 g (53%). LC-MS: Calculated for C₃₃H₃₇N₅O₄ is 567.69, Observed: 568.2 [M+1]⁺

### Step 8:

To a stirred solution of 11 (150 mg, 0.264 mmol) in MeOH (6 mL), was added p-toluenesulfonic acid monohydrate (151 mg, 0.793 mmol) at 0° C. and the reaction mixture stirred at room temperature for 16 h. The reaction mixture was diluted with DCM (30 mL) and washed with aqueous 10% NaHCO₃ solution (2×15 mL). The organic layer was separated, dried over anhydrous Na₂SO₄, filtered and concentrated under reduced pressure. The crude residue thus obtained was purified by reverse phase preparative HPLC (Column: Shimpack GIST C18 (150*20 mm) 5 μm; Eluents: 10 mM ammonium bicarbonate in water and ACN) to afford Compound 98 as a white solid. Yield: 0.022 g (16%). LC-MS: Calculated for C₂₈H₂₉N₅O₃ is 483.57, Observed: 484.2 [M+1]⁺. 1H-NMR (400 MHz, DMSO-d6): δ 7.68-7.65 (m, 4H), 7.52 (d, J=8.40 Hz, 2H), 7.36 (app d, J=0.80 Hz, 1H), 6.98 (d, J=8.40 Hz, 2H), 6.84 (app d, J=1.20 Hz, 1H), 6.77 (app d, J=1.20 Hz, 1H), 6.52 (app d, J=1.20 Hz, 1H), 5.69 (t, J=6.00 Hz, 1H), 5.52 (t, 1H, exchanges with D2O), 5.38 (t, J=5.60 Hz, 1H, exchanges with D2O), 5.19-5.13 (m, 1H), 4.97-4.91 (m, 1H), 4.42-4.38 (m, 2H), 3.97-3.93 (m, 2H), 3.87 (t, J=5.60 Hz, 2H), 3.38 (s, 3H), 1.51 (d, J=6.80 Hz, 3H). SFC: 93.4%; tR=3.90 min (Column: R,R-Whelk; Eluents: CO2 and 0.5% isopropyl amine in MeOH). SFC purity=93.4%

### Example A49: Synthesis of Compound 99

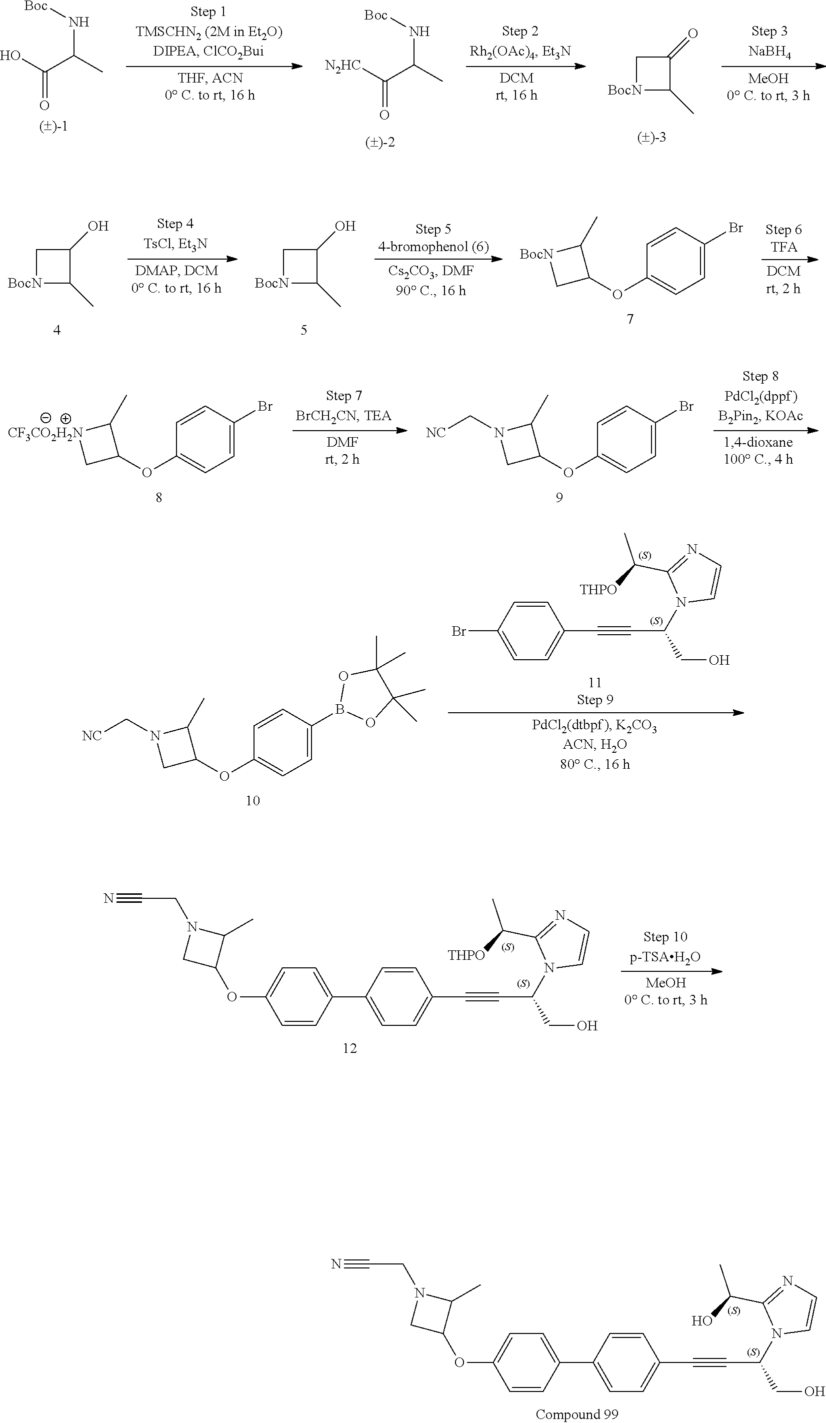

### Step-1:

To a stirred solution of Boc-DL-alanine (1, 7 g, 37.0 mmol) in THF (70 mL), were added DIPEA (10.96 mL, 62.9 mmol) and isobutyl chloroformate (9.60 mL, 74.0 mmol) at 0° C. and the reaction mixture stirred for 3 h at 0° C. Then, acetonitrile (8 mL) and (trimethylsilyl)diazomethane (2 M in Et2O, 27.7 mL, 55.5 mmol) were added at 0° C. and the stirring continued for 3 h. The resulting reaction mixture stirred at room temperature for 16 h. The reaction mixture was quenched with water (50 mL), extracted with EtOAc (2×50 mL). The combined organic layer was washed with brine solution (50 mL), dried over anhydrous Na₂SO₄, filtered and concentrated under reduced pressure. The crude product thus obtained was purified by using MPLC (manually packed cartridge; SiO₂ 100-200 mesh; 2-5% EtOAc in hexanes) to afford diazoketone 2 as a pale-yellow solid. Yield: 2.8 g (26%)

### Step 2:

To a stirred solution of 2 (2.8 g, 9.80 mmol) in DCM (10 mL), were added TEA (0.012 mL, 0.093 mmol) and rhodium (II) acetate dimer (87 mg, 0.196 mmol) at 0° C. and the resulting reaction mixture stirred at room temperature for 16 h. After completion, the reaction mixture was quenched with water (25 mL) and extracted with EtOAc (2×50 mL). The combined organic layer was dried over anhydrous Na₂SO₄ and concentrated under reduced pressure. The resulting crude mass was purified by using MPLC (manually packed cartridge; SiO₂ 100-200 mesh; 0-10% EtOAc in hexanes) to afford 3 as a colorless gum. Yield: 800 mg (40%).

### Step 3:

To a stirred solution of 3 (800 mg, 4.32 mmol) in MeOH (10 mL), was added NaBH₄ (245 mg, 6.48 mmol) in portions at 0° C. The resulting reaction mixture was stirred at room temperature for 3 h and the reaction mixture concentrated under reduced pressure. To the resulting residue, water (20 mL) was added and extracted with EtOAc (2×20 mL). The combined organic layer was dried over anhydrous Na₂SO₄, filtered and concentrated reduced pressure to afford 4 as a pale-yellow liquid. LC-MS: Calculated for C₉H₁₇NO₃ is 187.12; Observed: 132 [(M-Boc)+1]⁺ Yield: 600 mg (71%). The relative stereochemistry was not confirmed. The mixture was carried forward.

### Step 4:

To a stirred solution of 4 (600 gm, 3.20 mmol) in DCM (20 mL), were added Et3N (1.34 mL, 9.61 mmol), DMAP (39.1 mg, 0.320 mmol) and tosyl chloride (794 mg, 4.17 mmol) at 0° C. under nitrogen, and the resulting reaction mixture stirred at room temperature for 16 h. The reaction mixture was quenched with water (20 mL) and extracted with DCM (2×30 mL). The combined organic layer was dried over anhydrous Na₂SO₄, filtered and concentrated reduced pressure. The resulting crude mass was purified by using MPLC (manually packed cartridge; SiO2 100-200 mesh size; 20% EtOAc in hexane) to afford 5 as a light-yellow gum. Yield: 500 mg (46%). LC-MS: Calculated C₁₆H₂₃NO₅S is 341.42, Observed: 242.3 [(M-Boc)+1]⁺

### Step 5:

To a solution of 5 (500 mg, 1.464 mmol) in DMF (10 mL), were added Cs₂CO₃ (1431 mg, 4.39 mmol) and 4-bromophenol (6, 279 mg, 1.611 mmol) at room temperature and the resulting reaction mixture stirred at 90° C. for 16 h. The reaction mixture was quenched with water (20 mL) and extracted with EtOAc (2×30 mL). The combined organic layer was dried over anhydrous Na₂SO₄, filtered and concentrated reduced pressure. The crude mass was purified by using MPLC (manually packed cartridge; SiO₂ 100-200 mesh size; 20% EtOAc in hexane) to afford 7 as a colorless gum. Yield: 400 mg (53%). LC-MS: Calculated C₁₅H₂₀BrNO₃ is 342.23, Observed: 242.0 [M-Boc]⁺ and 243.9 [(M-Boc)+2]⁺

### Step 6:

To a stirred solution of 7 (400 mg, 1.169 mmol) in DCM (4 mL), was added TFA (0.572 mL, 7.48 mmol) at 0° C. under nitrogen, and the resulting reaction mixture stirred at room temperature for 2 h. The volatiles in the reaction mixture were removed under reduced pressure to afford 8 as a brown gum. Yield=400 mg (86%). LC-MS: Calculated C₁₀H₁₂BrNO is 242.11, Observed: 242.0 [M]⁺ and 244.1 [M+2]⁺

### Step-7:

To a solution of 8 (400 mg, 1.123 mmol) in DMF (4 mL), were added TEA (0.939 mL, 6.74 mmol) and bromoacetonitrile (0.117 mL, 1.685 mmol) at 0° C. under nitrogen, and the resulting reaction mixture stirred at room temperature for 2 h. The reaction mixture was quenched with water (20 mL) and extracted with DCM (2×10 mL). The combined organic layer was dried over anhydrous Na₂SO₄, filtered and concentrated under reduced pressure. to afford 9 as a brown gum. LC-MS: Calculated C₁₂H₁₃BrN₂O is 281.15, Observed: 281.2 [M]⁺ and 283.2 [M+2]⁺. Yield: 320 mg (87%)

### Step-8:

To a solution of 9 (300 mg, 1.067 mmol) in 1,4-dioxane (3 mL), were added potassium acetate (314 mg, 3.20 mmol) and bis(pinacolato)diboron (406 mg, 1.601 mmol) at room temperature and the resulting mixture purged with nitrogen for 5 min. To this reaction mixture, PdCl₂(dppf) (39 mg, 0.053 mmol) was added and again purged with nitrogen for another 5 min. The resulting reaction mixture was then stirred at 100° C. for 4 h. The reaction mixture was cooled to room temperature, quenched with water (20 mL), extracted with EtOAc (2×20 mL). The organic layer was washed with brine (10 mL), dried over anhydrous Na₂SO₄, filtered and concentrated under reduced pressure. The resulting crude mass was purified by using MPLC (manually packed cartridge; SiO₂ 100-200 mesh; 0-20% EtOAc in hexanes) to afford 10 as a colorless gum. Yield: 360 mg (98%). LC-MS: Calculated C₁₈H₂₅BN₂O₃ is 328.22, Observed: 329.3 [M+1]⁺

### Step-9:

To a stirred solution of 11 (250 mg, 0.596 mmol) in acetonitrile (2.25 mL) and water (0.25 mL), were added 10 (294 mg, 0.894 mmol) and K₂CO₃ (247 mg, 1.789 mmol) at room temperature, and the reaction mixture purged with nitrogen for 5 min. To this reaction mixture, PdCl2(dtbpf) (19.43 mg, 0.030 mmol) was added and the purging continued for another 5 min. The resulting reaction mixture was heated to 80° C. for 16 h. The reaction mixture was quenched with water (20 mL), extracted with EtOAc (2×20 mL). The combined organic layer was washed with brine solution (10 mL), dried over anhydrous Na₂SO₄, filtered and concentrated under reduced pressure. The resulting crude product was purified by using MPLC (manually packed SiO₂ cartridge, 100-200 mesh size; 0-10% MeOH in DCM) to afford 12 as a colorless gum. Yield: 180 mg (51%). LC-MS: Calculated for C₃₂H₃₆N₄O₄ is 540.66, Observed: 541.3 [M+1]⁺

### Step-10:

To a stirred solution of 12 (180 mg, 0.333 mmol) in MeOH (5 mL), was added p-toluene sulfonic acid monohydrate (190 mg, 0.999 mmol) at 0° C. under nitrogen, and the resulting reaction mixture stirred at room temperature for 3 h. The volatiles were evaporated under reduced pressure; the resulting residue was basified with 10% NaHCO₃ solution (10 mL) and extracted with 10% MeOH in DCM (2×10 mL). The combined organic extract was dried over anhydrous Na₂SO₄, filtered and concentrated under reduced pressure. The crude thus obtained was purified by reverse phase preparative HPLC (Column: Shimpack-(150*20) mm, 5 μm; Eluents: 10 mM ammonium bicarbonate in water and acetonitrile) to obtain Compound 99 as an off-white solid. Yield=15 mg (10%). LC-MS: Calculated for C₂₇H₂₈N₄O₃ is 456.55, Observed: 457.2 [M+1]⁺. 1H-NMR (400 MHz, DMSO-d6): δ 7.67-7.63 (m, 4H), 7.52 (d, J=8.40 Hz, 2H), 7.36 (d, J=1.20 Hz, 1H), 6.99-6.97 (m, 2H), 6.85 (d, J=0.80 Hz, 1H), 5.69 (t, J=6.00 Hz, 1H), 5.53 (t, J=5.60 Hz, 1H, exchanges with D2O), 5.38 (d, J=5.60 Hz, 1H, exchanges with D2O), 4.97-4.91 (m, 1H), 4.52 (q, J=6.00 Hz, 1H), 3.88-3.79 (m, 5H), 3.38 (t, J=6.00 Hz, 1H), 2.93 (t, J=6.40 Hz, 1H), 1.51 (d, J=6.40 Hz, 3H), 1.30-1.28 (m, 3H).

SFC: 91.85%; tR=2.96 min (Column: LUX-I-Amylose3; Eluents: CO₂ and 0.55% isopropyl amine in MeOH). Mixture of diastereomers; racemic in tail

### Example A50: Synthesis of Compound 100

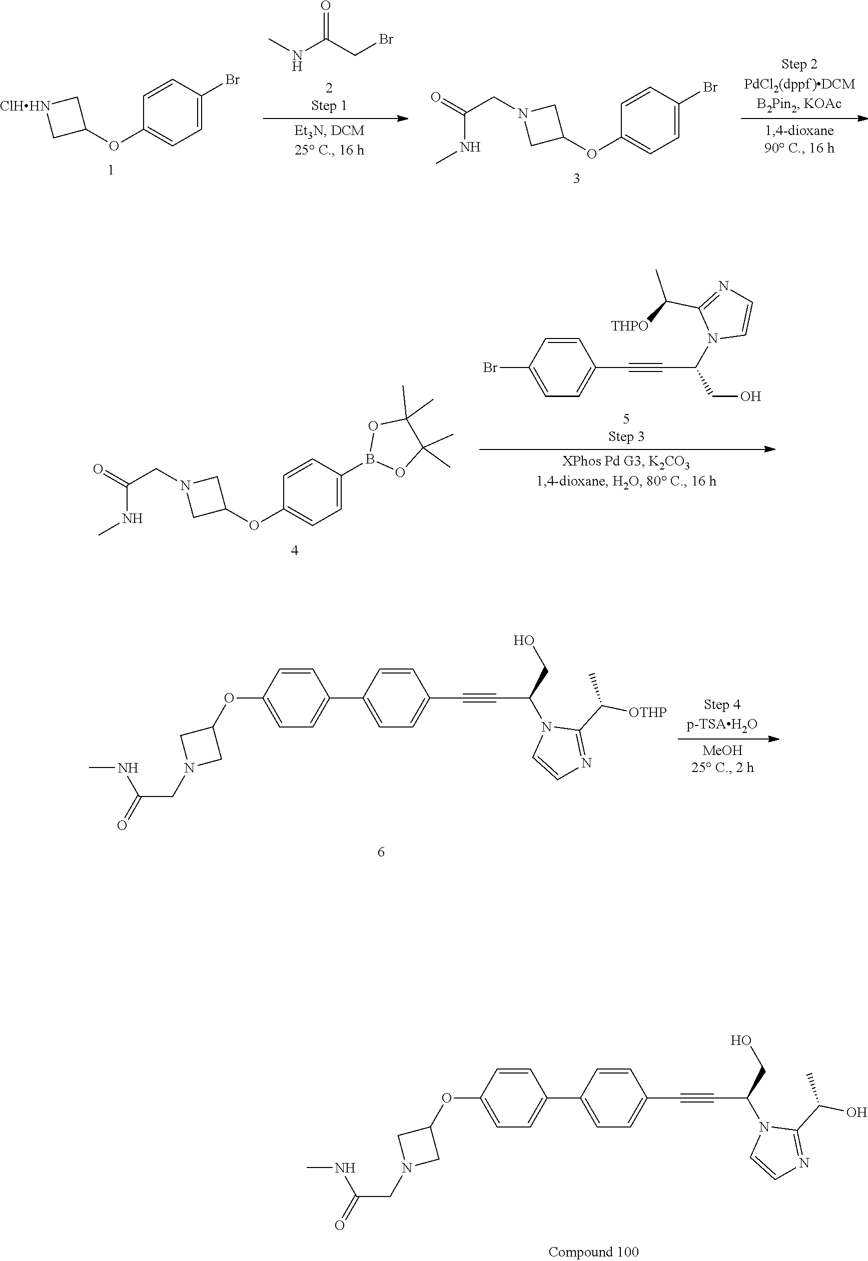

### Step 1:

To a stirred solution of 1 (2.0 g, 7.56 mmol) in anhydrous DMF (15 mL), were added TEA (3.15 mL, 22.68 mmol) and 2-bromo-N-methylacetamide (2, 1.264 g, 8.32 mmol) at 25° C. The reaction mixture was stirred at 25° C. for 16 h. The reaction was quenched with water (20 mL) and extracted with EtOAc (25 mL×2). The combined organic layer was washed with brine (20 mL), dried over anhydrous Na₂SO₄, filtered and concentrated under reduced pressure. The crude residue thus obtained, was purified by using MPLC (manually packed SiO₂ cartridge, 230-400 mesh size; 90% EtOAc in hexanes) to afford 3 as a white solid.

Yield: 1.3 g (51%)

LC-MS: Calculated for C₁₂H₁₅BrN₂O₂ is 299.17 Observed: 299.9 [M]⁺ and 300.9 [M+2]⁺

### Step-2:

To a stirred solution of 3 (1.1 g, 3.68 mmol) in 1,4-dioxane (15 mL), were added bis(pinacolato)diboron (1.214 g, 4.78 mmol) and potassium acetate (1.083 g, 11.03 mmol) at room temperature. The reaction mixture was purged with nitrogen for 10 min, following which PdCl₂(dppf)·CH₂Cl₂ (0.300 g, 0.368 mmol) was added. The mixture was purged with nitrogen for 10 min and then stirred at 90° C. for 16 h. The reaction mixture was filtered through a pad of Celite and washed with EtOAc (25 mL). The combined filtrate was dried over anhydrous Na₂SO₄, filtered and concentrated under reduced pressure. The resulting crude residue was purified by using MPLC (manually packed SiO₂ cartridge, 230-400 mesh; 3% MeOH in DCM) to afford 4 as a brown liquid. LCMS showed 77% purity; the product was taken to the next step without further purification.

Yield: 1.1 g (67%). LC-MS: Calculated for C₁₈H₂₇BN₂O₄ is 346.23, Observed: 347.0 [M+1]⁺

### Step-3:

To a stirred solution of 4 (643 mg, 1.431 mmol) in mixture of 1,4-dioxane (16 mL) and water (4 mL), were added 5 (500 mg, 1.192 mmol) and K₂CO₃ (330 mg, 2.385 mmol) at room temperature. The reaction mixture was degassed for 10 min, following which XPhos Pd G3 (202 mg, 0.238 mmol) was added. The mixture was again degassed for 10 min and then stirred at 80° C. for 16 h. The reaction mixture was passed through a pad of Celite and washed with EtOAc (20 mL). The combined filtrate was dried over anhydrous Na₂SO₄, filtered and concentrated under reduced pressure. The crude residue, thus obtained, was purified using MPLC (manually packed SiO₂ cartridge, 230-400 mesh size; 20% MeOH in EtOAc) to afford 6 as a brown solid. LCMS showed 77% purity; product was taken to the next step.

Yield: 400 mg (46%)

LC-MS: Calculated for C₃₂H₃₈N₄O₅ is 558.68, Observed: 559.2 [M+1]⁺

### Step-4:

To a stirred solution of 6 (400 mg, 0.551 mmol) in MeOH (10 mL), was added p-toluene sulfonic acid monohydrate (210 mg, 1.103 mmol) at 0° C. and the resulting reaction mixture stirred at 25° C. for 2 h. The reaction mixture was concentrated and basified with sat. NaHCO₃ solution (25 mL). The aqueous layer was extracted with 10% MeOH in DCM (20 mL×3). The combined organic extract was dried over anhydrous Na₂SO₄, filtered and concentrated under reduced pressure. The resulting crude residue was purified by reverse phase preparative HPLC (Column: YMC Triart C18 (20*250 mm) 5 μm; Eluents:10 mM ammonium bicarbonate in water and ACN) to obtain Compound 100 as a white solid.

Yield: 60 mg (23%)

LC-MS: Calculated for C₂₇H₃₀N₄O₄ is 474.56, Observed: 475.2 [M+1]⁺

1H-NMR (400 MHz, DMSO-d6): δ 7.69-7.62 (m, 5H, 1H exchanges with D2O), 7.52 (d, J=8.40 Hz, 2H), 7.35 (app d, J=1.20 Hz, 1H), 6.92 (d, J=8.80 Hz, 2H), 6.84 (app d, J=0.80 Hz, 1H), 5.69 (t, J=6.00 Hz, 1H), 5.53 (t, J=6.00 Hz, 1H, exchanges with D2O), 5.37 (d, J=5.60 Hz, 1H, exchanges with D2O), 4.97-4.91 (m, 1H), 4.89-4.83 (m, 1H), 3.88-3.82 (m, 4H), 3.21-3.17 (m, 2H), 3.10 (s, 2H), 2.59 (d, J=4.80 Hz, 3H), 1.51 (d, J=6.80 Hz, 3H).

SFC: 96.4%; tR=2.95 min (Column: l-cellulose-Z; Eluents: 0.5% isopropyl amine in MeOH and CO2). Single isomer with SFC purity 96.4%

### Example A51: Synthesis of Compound 101

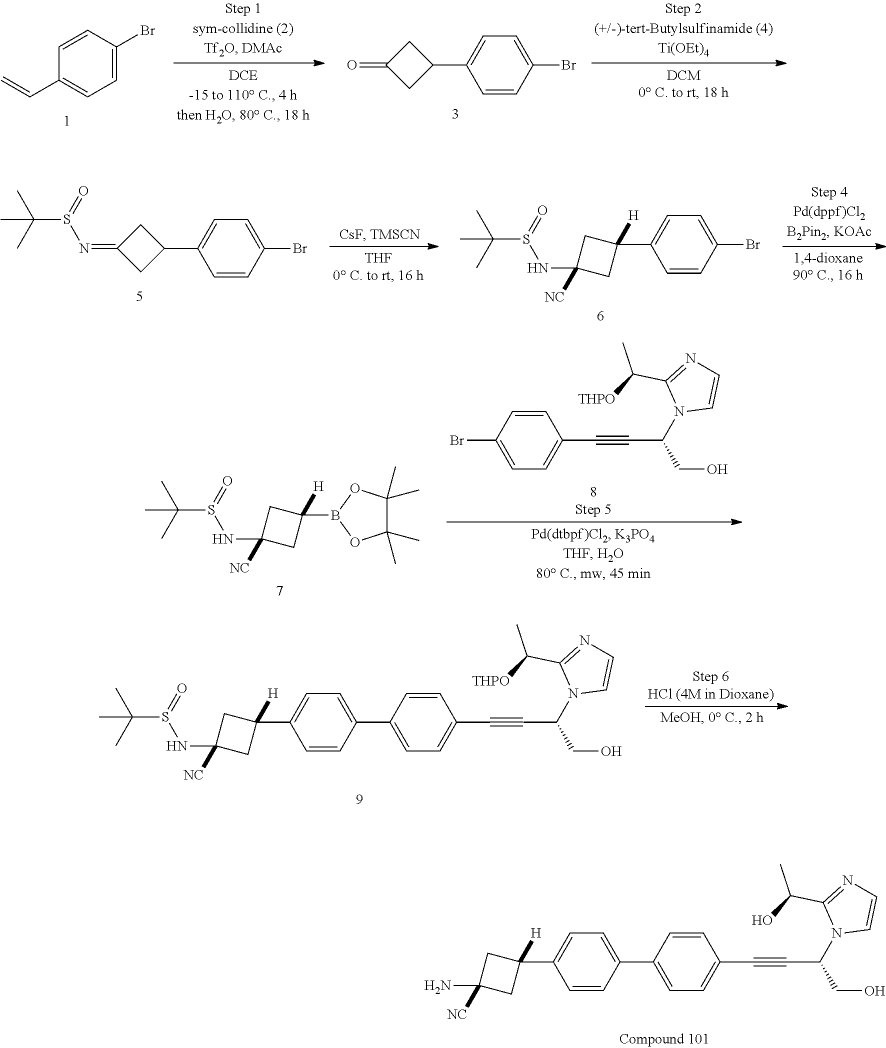

### Step 1:

To a stirred solution of N,N-dimethylacetamide (51.2 mL, 546 mmol) in DCE (140 mL), was added a solution of trifluoromethanesulfonic anhydride (92 mL, 546 mmol) in DCE (140 mL) at −15° C. dropwise. The reaction mixture was stirred at −15° C. for 30 min. To this reaction mixture, a solution of 4-bromostyrene (1, 71.4 mL, 546 mmol) in DCE (140 mL) followed by a solution of sym-collidine (2, 72.8 mL, 546 mmol) in DCE (140 mL) were added. The reaction mixture was heated at 110° C. for 4 h. The reaction temperature was brought to 80° C., water (200 mL) was added and the stirring was continued at 80° C. for 18 h. The reaction mixture was diluted with water (400 mL). This was extracted with DCM (3×800 mL). The combined organic layer was washed with brine (400 mL), dried over anhydrous sodium sulfate, filtered and concentrated under reduced pressure to get the crude residue. The crude residue was purified by using MPLC (manually packed cartridge, SiO₂ 230-400 mesh; eluents: 5% EtOAc in hexanes) to get 3 as a yellow solid.

Yield: 50 g (41%)

### Step 2:

To a stirred solution of 3 (3 g, 13.33 mmol) and (±/−)-tert-Butylsulfinamide (4, 1.938 g, 15.99 mmol) in DCM (60 mL), was added titanium (IV) ethoxide (5.63 mL, 26.7 mmol) dropwise at 0° C., and the reaction mixture stirred at room temperature for 18 h. The reaction mixture was quenched with water (25 mL). The insoluble solid was filtered off and the filtrate was diluted with DCM (20 mL). The aqueous layer was separated and extracted with DCM (20 mL). The combined organic layer was dried over anhydrous sodium sulfate, filtered and concentrated under reduced pressure to get 5 as a pale-yellow gum. The crude product was taken to the next step without any purification.

Yield: 4 g (crude product weight)

LC-MS: Calculated for C₁₄H₁₈BrNOS is 328.26, Observed: 328.0 [M]⁺ and 330.0 [M+2]⁺

### Step 3:

To a stirred solution of 5 (4 g, 12.19 mmol) in THF (40 mL), were added cesium fluoride (3.70 g, 24.37 mmol) and trimethylsilyl cyanide (2.295 mL, 18.28 mmol) at 0° C., and the reaction mixture stirred at 0° C. for 2 h. The reaction mixture was slowly warmed to room temperature and stirred for 16 h. The reaction mixture was quenched with water (30 mL) and extracted with EtOAc (2×40 mL). The combined organic layer was washed with brine (10 mL), dried over anhydrous sodium sulfate, filtered and concentrated under reduced pressure. The crude residue thus obtained was purified by using MPLC (manually packed cartridge, SiO₂ 100-200 mesh; eluents: as 25% EtOAc in hexanes) to afford 6 as a white solid.

Yield: 3 g (67%)

LC-MS: Calculated for C₁₅H₁₉BrN₂OS is 355.29, Observed: 355.3 [M]⁺ and 357.2 [M+2]⁺

The structure of compound 6 was confirmed by NOESY.

### Step 4:

To a stirred solution of 6 (2.8 g, 7.88 mmol) in 1,4-dioxane (35 mL), were added bis(pinacolato)diboron (3.00 g, 11.82 mmol) and potassium acetate (2.320 g, 23.64 mmol) at room temperature, and a stream of nitrogen gas bubbled through the reaction mixture for 15 min. Then, PdCl₂(dppf) (0.577 g, 0.788 mmol) was added and the reaction mixture heated at 90° C. for 16 h. The reaction mixture was filtered through a pad of Celite; the filtrate was concentrated under reduced pressure. The crude residue, thus obtained, was purified by using MPLC (manually packed cartridge, SiO₂ 230-400 mesh; eluents: 25% EtOAc in hexanes) to afford 7 as an off-white solid.

Yield: 1.3 g (35%)

LC-MS: Calculated for C₂₁H₃₁BN₂O₃S is 402.36, Observed: 403.4 [M+1]⁺

### Step 5:

To a stirred solution of 8 (350 mg, 0.835 mmol) and 7 (504 mg, 1.252 mmol) in a mixture of THF (12 mL) and water (1.3 mL), were added potassium phosphate tribasic (532 mg, 2.504 mmol), and a stream of nitrogen gas bubbled through the reaction mixture for 15 min. Then, PdCl₂(dtbpf) (54.4 mg, 0.083 mmol) was added and the reaction mixture irradiated at 80° C. in Biotage microwave reactor for 45 min. One more batch was carried out using 350 mg of 8. Both the batches were mixed for workup and purification. The reaction mixture was filtered through a pad of Celite. The bed was washed with EtOAc (10 mL), the filtrate combined and concentrated under reduced pressure. The crude residue, thus obtained, was purified by reverse phase purification (column: Redisep Gold, C18 SiO2; eluents: 0.1% formic acid in water and ACN) to obtain 9 as a light-brown gum.

Yield: 180 mg (combined yield for two batches)

LC-MS: Calculated for C₃₅H₄₂N₄O₄S is 614.81, Observed: 615.3 [M+1]⁺

### Step 6:

To a stirred solution of 9 (180 mg, 0.293 mmol) in MeOH (5 mL), was added HCl (4 M in 1,4-dioxane, 0.366 mL, 1.464 mmol) at 0° C., and the reaction mixture stirred at 0° C. for 2 h. The reaction mixture was concentrated under reduced pressure. The resulting residue was suspended in 10% NaHCO₃ solution (20 mL), and extracted with DCM (2×30 mL). The combined organic layer was dried over anhydrous Na₂SO₄, filtered and the filtrate was concentrated under reduced pressure to get the crude residue. The crude residue was purified by reverse phase preparative HPLC (column: Shimpack C18 (150*20 mm) 5 μm; eluents: 10 mM ammonium bicarbonate in water and ACN) to afford Compound 101 as a white solid.

Yield: 30 mg (19%)

LC-MS: Calculated for C₂₆H₂₆N₄O₂ is 426.52, Observed: 427.0 [M+1]⁺

1H-NMR (400 MHz, DMSO-d6): δ 7.69 (d, J=8.40 Hz, 2H), 7.65 (d, J=8.00 Hz, 2H), 7.54 (d, J=8.00 Hz, 2H), 7.42 (d, J=8.00 Hz, 2H), 7.36 (s, 1H), 6.84 (s, 1H), 5.75 (m, 1H), 5.54 (t, J=5.60 Hz, 1H, exchanges with D2O), 5.38 (d, J=5.60 Hz, 1H, exchanges with D2O), 4.97-4.91 (m, 1H), 3.87 (t, J=5.60 Hz, 2H), 3.48-3.33 (m, 1H), 2.93-2.88 (m, 2H), 2.87 (br s, 1H, exchanges with D2O), 2.33-2.25 (m, 2H), 1.51 (d, J=6.40 Hz, 3H). One less proton observed in 1H NMR.

SFC: 80.7% and 12.2%; tR=3.20 min and 3.45 min respectively (column: Whelk-(R,R); Eluents: CO2 and 0.5% isopropyl amine in MeOH). SFC-MS showed 427.1 [M+1]⁺ at two different tR. Major cis-geometry in tail part based on 1H NMR. Mixture of diastereomers; racemic at tail.

### Example A52: Synthesis of Compound 102

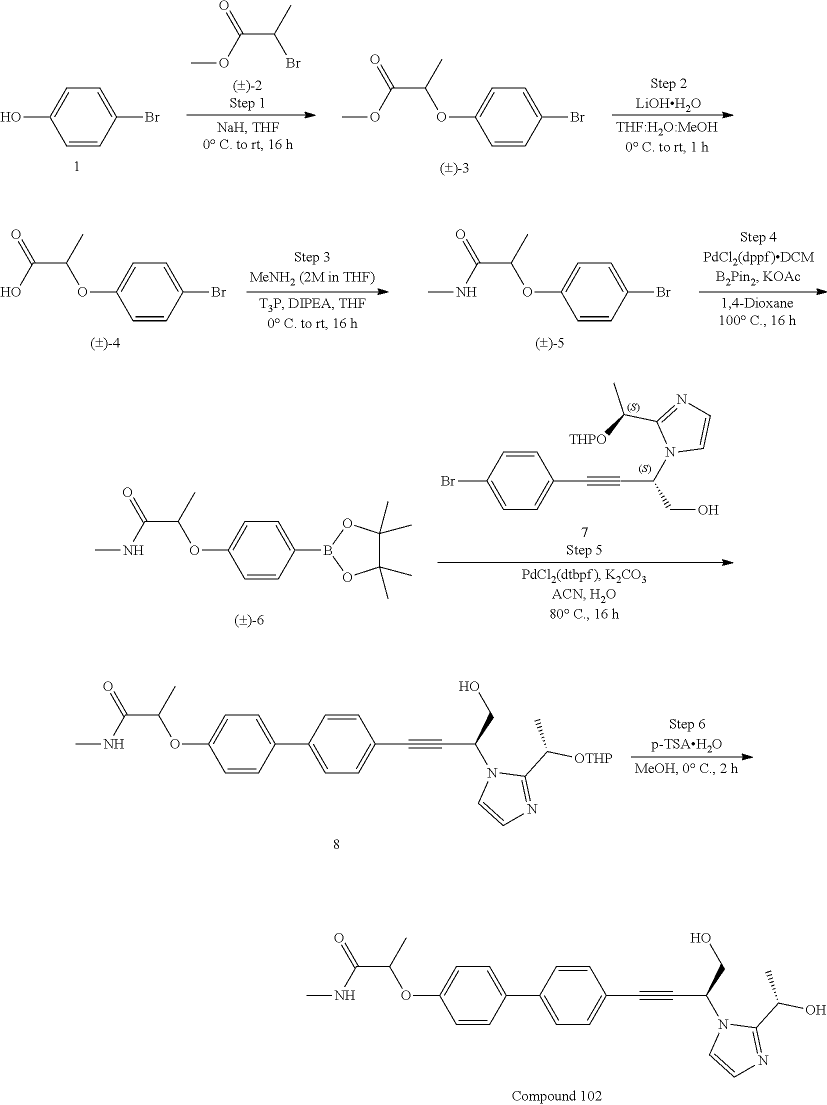

### Step-1:

To a solution of 4-bromophenol (1, 5 g, 28.9 mmol) in THF (50 mL), was added NaH (60% in mineral oil, 1.73 g, 43.4 mmol) at 0° C. and stirred for 30 min. To this reaction mixture, methyl 2-bromopropanoate ((±)-2, 4.84 mL, 43.4 mmol) was added drop wise and the resulting reaction mixture stirred at room temperature for 16 h. The reaction mixture was cooled to 0° C., quenched with ice-cold water (20 mL) and extracted with EtOAc (50 mL×2). The combined organic layer was dried over anhydrous Na₂SO₄, filtered and concentrated under reduced pressure. The resulting crude residue was purified by MPLC (using manually packed SiO2 cartridge, 100-200 mesh size; 15% EtOAc in hexanes) to afford (±)-3 as a colorless liquid.

Yield=6.7 g (76%)

### Step-2:

To a stirred solution of (±)-3 (6.3 g, 24.32 mmol) in THF:MeOH:H₂O (3:2:1; 50 mL), was added LiOH·H₂O (3.06 g, 72.9 mmol) at room temperature and stirred for 1 h. The volatiles were removed under reduced pressure. The crude residue was acidified with 1.5 N HCl until pH˜2. The solid obtained was filtered and dried to afford (±)-4 as an off-white solid. Yield=4.5 g (74%). LC-MS: Calculated for C₉H9BrO3 is 245.072, Observed: 245.0 [M]⁻ and 243.0 [M−2]⁻

### Step-3:

To a stirred solution of (±)-4 (3 g, 12.24 mmol) in DMF (25 mL), were added DIPEA (11.14 mL, 61.2 mmol), T3P (50% in EtOAc, 11.02 mL, 18.36 mmol) at 0° C. and stirred for 15 min. To this reaction mixture, MeNH₂ (2M in THF, 12.24 mL, 24.28 mmol) was added at 0° C. The resulting reaction mixture was stirred at room temperature for 16 h. The reaction mixture was quenched with water (20 mL) and extracted with EtOAc (2×100 mL). The combined organic layer was dried over anhydrous Na₂SO₄, filtered and concentrated under reduced pressure to afford the crude residue. The crude residue was purified by MPLC (using manually packed SiO2 cartridge, 100-200 mesh size; 30% EtOAc in hexanes) to afford (±)-5 as a colorless liquid. Yield: 3 g (90%)

### Step-4:

To a stirred solution of (±)-5 (3.0 g, 11.62 mmol) in dioxane (35 mL), were added potassium acetate (2.05 g, 34.9 mmol) and bis(pinacolato)diboron (4.43 g, 17.43 mmol) at room temperature. The reaction mixture was degassed with nitrogen for 10 min. To this reaction mixture, was added PdCl2(dppf)·CH2Cl2 adduct (0.475 g, 0.581 mmol) and the degassing continued for 2 min. The reaction mixture was stirred at 100° C. for 16 h. The inorganic solids were filtered through Celite pad and washed with EtOAc (200 mL). The filtrate was concentrated under reduced pressure. The resulting crude residue was purified by MPLC (using manually packed SiO2 cartridge, 100-200 mesh size; 70% EtOAc in hexanes) to afford (±)-6 as an off-white solid. Yield: 2.5 g (57%)

### Step-5:

To a solution of 7 (1.5 g, 3.58 mmol) in acetonitrile (15 mL) and water (15 mL), were added (±)-6 (1.64 g, 5.37 mmol) and K₂CO₃ (1.5 g, 10.73 mmol) at room temperature. The resulting mixture was purged with nitrogen for 15 min. To this reaction mixture, PdCl₂(dtbpf) (0.233 g, 0.358 mmol) was added and the purging continued for 5 min. The reaction mixture was stirred at 80° C. for 16 h. To the reaction mixture, water (100 mL) was added and extracted with 10% MeOH in DCM (2×100 mL). The combined organic layer was dried over anhydrous Na₂SO₄, filtered and concentrated under reduced pressure. The resulting crude residue was purified by MPLC (using manually packed cartridge SiO2 230-400 mesh size; 5% MeOH in DCM) to afford 8 as pale brown solid. Yield=1.3 g (610%). LC-MS: Calculated for C30H35N3O5 is 517.626, Observed: 518.3 [M+1]⁺

### Step-6:

To a stirred solution of 8 (1.2 g, 2.318 mmol) in MeOH (15 mL), was added p-toluene sulfonic acid monohydrate (1.32 g, 6.95 mmol) at 0° C. and the reaction mixture stirred at the room temperature for 2 h. The reaction mixture was quenched with sat. sodium bicarbonate solution (10 mL) and extracted with 10% MeOH in DCM (100 mL×2). The combined organic layer was dried over anhydrous Na₂SO₄, filtered and concentrated under reduced pressure. The crude residue was purified by MPLC (using manually packed SiO2 cartridge, 100-200 mesh size; 5% MeOH in DCM) to afford Compound 102 as a pale-brown solid.

Yield: 0.5 g (49%)

LCMS: Calculated for C25H27N3O4 is 433.508; Observed: 434.2 [M+1]⁺

1H-NMR (400 MHz, DMSO-d6): δ 8.05-8.03 (m, 1H), 7.67-7.63 (m, 4H), 7.52 (d, J=8.40 Hz, 2H), 7.35 (app d, J=1.20 Hz, 1H), 7.01 (d, J=8.80 Hz, 2H), 6.84 (app d, J=1.20 Hz, 1H), 5.68 (t, J=5.60 Hz, 1H), 5.54-5.51 (m, 1H, exchanges with D2O), 5.30 (d, 1H, exchanges with D2O), 4.96-4.91 (m, 1H), 4.76-4.71 (m, 1H), 3.87 (t, J=6.00 Hz, 2H), 2.61 (d, J=4.40 Hz, 3H), 1.51 (d, J=6.80 Hz, 3H), 1.45 (d, J=6.40 Hz, 3H). The product was a mixture of diastereomers; racemic at tail part (l-Cellulose-Z_0.5% IPAm in MeOH t_{R}=5.54 and 6.28 min)

### Example A53: Synthesis of Compound 103

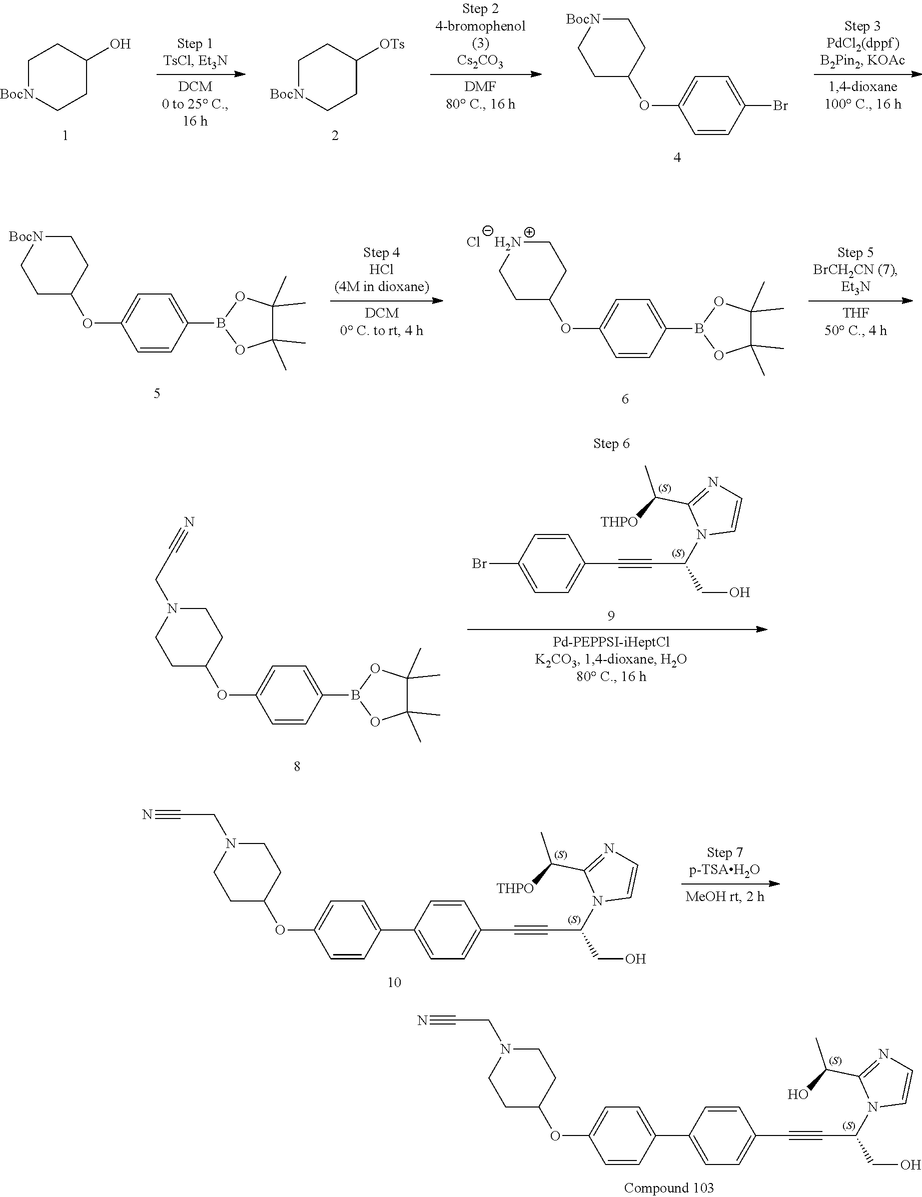

### Step 1:

To a stirred solution of tert-butyl 4-hydroxypiperidine-1-carboxylate (1, 5 g, 24.84 mmol) in DCM (100 mL), were added TEA (10.47 mL, 74.5 mmol) and tosyl chloride (7.10 g, 37.3 mmol) at 0° C. and the reaction mixture stirred at 25° C. for 16 h. The reaction was quenched with water (25 mL) and extracted with DCM (20 mL×2). The combined organic layer was washed with 10% NaHCO₃ (2×15 mL), brine (25 mL), dried over anhydrous Na₂SO₄, filtered and concentrated under reduced pressure. The crude residue, thus obtained, was purified by MPLC (using manually packed SiO₂ cartridge 230-400 mesh size; 15% EtOAc in hexanes) to afford 2 as a white solid.

Yield: 9 g (100%).

LC-MS: Calculated for C₁₇H₂₅NO₅S is 355.449 Observed: 256.2 [M-Boc+1]⁺.

### Step-2:

To a stirred solution of 2 (10.48 g, 29.5 mmol) in DMF (50 mL), were added cesium carbonate (19.21 g, 59 mmol) and 4-bromophenol (3, 3.4 g, 34.7 mmol) at room temperature. The reaction mixture was stirred at 80° C. for 16 h. The reaction was quenched with cold water (20 mL) and extracted with EtOAc (25 mL×2). The combined organic layer was washed with brine (30 mL), dried over anhydrous Na₂SO₄, filtered and concentrated under reduced pressure. The crude residue thus obtained, was purified by MPLC (using manually packed SiO₂ cartridge 230-400 mesh; 5% EtOAc in hexanes) to afford 4 as a white solid.

Yield: 4.0 g (38%). LC MS: Calculated for C₁₆H₂₂BrNO₃ is 356.26, Observed: 256.2 [M-Boc]⁺ and 258.2 [M-Boc+2]⁺

### Step-3:

To a stirred solution of 4 (4 g, 11.23 mmol) in 1,4-dioxane (50 mL), were added bis(pinacolato)diboron (4.28 g, 16.84 mmol) and potassium acetate (3.31 g, 33.7 mmol) at room temperature. The reaction mixture was purged with nitrogen for 5 min, following which PdCl₂(dppf) (0.822 g, 1.123 mmol) was added. The mixture was purged with nitrogen for 10 min and then stirred at 100° C. for 16 h. The reaction mixture was filtered through a pad of Celite and washed with EtOAc (20 mL). The combined filtrate was dried over anhydrous Na₂SO₄, filtered and concentrated under reduced pressure to get crude residue. The resulting crude residue was purified by MPLC (using manually packed SiO₂ cartridge, 230-400 mesh; 15% EtOAc in hexanes) to afford 5 as a pale-yellow solid. LCMS showed 82% purity; taken to the next step without further purification. Yield: 5.5 g (100%). LC-MS: Calculated for C₂₂H₃₄BNO₅ is 403.33, Observed: 304.4 [M-Boc+1]⁺

### Step-4:

To a stirred solution of 5 (5.5 g, 13.64 mmol) in anhydrous DCM (50 mL), was added hydrochloric acid (4 M in dioxane, 6.82 mL, 27.3 mmol) at 0° C. The resulting mixture was stirred at 25° C. for 4 h. The reaction mixture was concentrated under reduced pressure. The crude mass was triturated with hexane (20 mL) to obtain 6 as an off-white solid.

Yield: 4.5 g (86%)

LC-MS: Calculated for C₁₇H₂₇BNO₃⁺ is 304.21, Observed: 304.2 [M]⁺

### Step-5:

To a stirred solution of 6 (4.5 g, 13.25 mmol) in anhydrous THF (50 mL), were added triethylamine (9.31 mL, 66.2 mmol) and bromoacetonitrile (7, 1.85 mL, 26.5 mmol) at 25° C., and the reaction mixture stirred at 50° C. for 4 h. The reaction was quenched with water (15 mL) and extracted with EtOAc (20 mL×2). The combined organic layer was washed with brine (15 mL), dried over anhydrous Na₂SO₄, filtered and concentrated under reduced pressure to obtain compound 8 as a colourless liquid.

Yield: 3.9 g (80%)

LC-MS: Calculated for C₁₉H₂₇BN₂O₃ is 342.25, Observed: 343.1 [M+1]⁺

### Step-6:

To a stirred solution of 8 (392 mg, 1.145 mmol) in 1,4-dioxane (12 mL) and water (4 mL), were added 9 (400 mg, 0.954 mmol) and K₂CO₃ (395 mg, 2.86 mmol) at room temperature. The reaction mixture was purged for 10 min, following which [1,3-Bis(2,6-Diisopropylphenyl)imidazol-2-ylidene](3-chloropyridyl)palladium(II) dichloride (Pd-PEPPSI-iHeptCl, 46.4 mg, 0.048 mmol) was added, purging continued for 10 min and then reaction mixture stirred at 80° C. for 16 h. The reaction mixture was passed through a pad of Celite and washed with EtOAc (15 mL). The filtrate was dried over anhydrous Na₂SO₄, filtered and concentrated under reduced pressure to get crude residue. The crude residue thus obtained, was purified by using MPLC (using manually packed SiO₂ cartridge, 230-400 mesh; 20% MeOH in DCM) to afford 10 as a brown colour solid.

Yield: 400 mg (73%)

LC-MS: Calculated for C₃₃H₃₈N₄O₄ is 554.69, Observed: 555.2 [M+1]⁺

### Step-7:

To a stirred solution of 10 (400 mg, 0.721 mmol) in MeOH (10 mL), was added p-toluenesulfonic acid monohydrate (274 mg, 1.442 mmol) at 0° C., and the resulting reaction mixture stirred at 25° C. for 2 h. The reaction mixture was then concentrated and basified with sat. NaHCO₃ solution (25 mL). The aqueous layer was extracted with 10% MeOH in DCM (20 mL×3). The combined organic extract was dried over anhydrous Na₂SO₄, filtered and concentrated under reduced pressure. The resulting crude residue was purified by reverse-phase preparative HPLC (Column: ZORBAXc18 (50*21.2 μm), 5 μm; Eluents:10 mM ammonium bicarbonate in water and ACN) to obtain Compound 103 as a white solid.

Yield: 85 mg (25%)

LC-MS: Calculated for C₂₈H₃₀N₄O₃ is 470.57 Observed: 471.4 [M+1]⁺

1H-NMR (400 MHz, DMSO-d6): δ 7.67-7.62 (m, 4H), 7.51 (dd, J=2.00, 6.80 Hz, 2H), 7.36 (app d, J=1.20 Hz, 1H), 7.07 (dd, J=2.00, 6.80 Hz, 2H), 6.84 (app d, J=1.60 Hz, 1H), 5.69 (t, J=6.00 Hz, 1H), 5.53 (t, J=5.60 Hz, 1H, exchanges with D2O), 5.37 (d, J=5.60 Hz, 1H, exchanges with D2O), 4.96-4.93 (m, 1H), 4.51-4.47 (m, 1H), 3.87 (t, J=6.00 Hz, 2H), 3.84 (s, 2H), 2.74-2.73 (m, 2H), 2.47-2.42 (m, 2H, merges with solvent peak), 2.02-1.98 (m, 2H), 1.73-1.68 (m, 2H), 1.51 (d, J=6.40 Hz, 3H). Single isomer with SFC purity 98.4% (LUX Amylose-1_0.5% IPAm in MeOH; Single isomer based on SFC-MS)

### Example A54: Synthesis of Compound 104

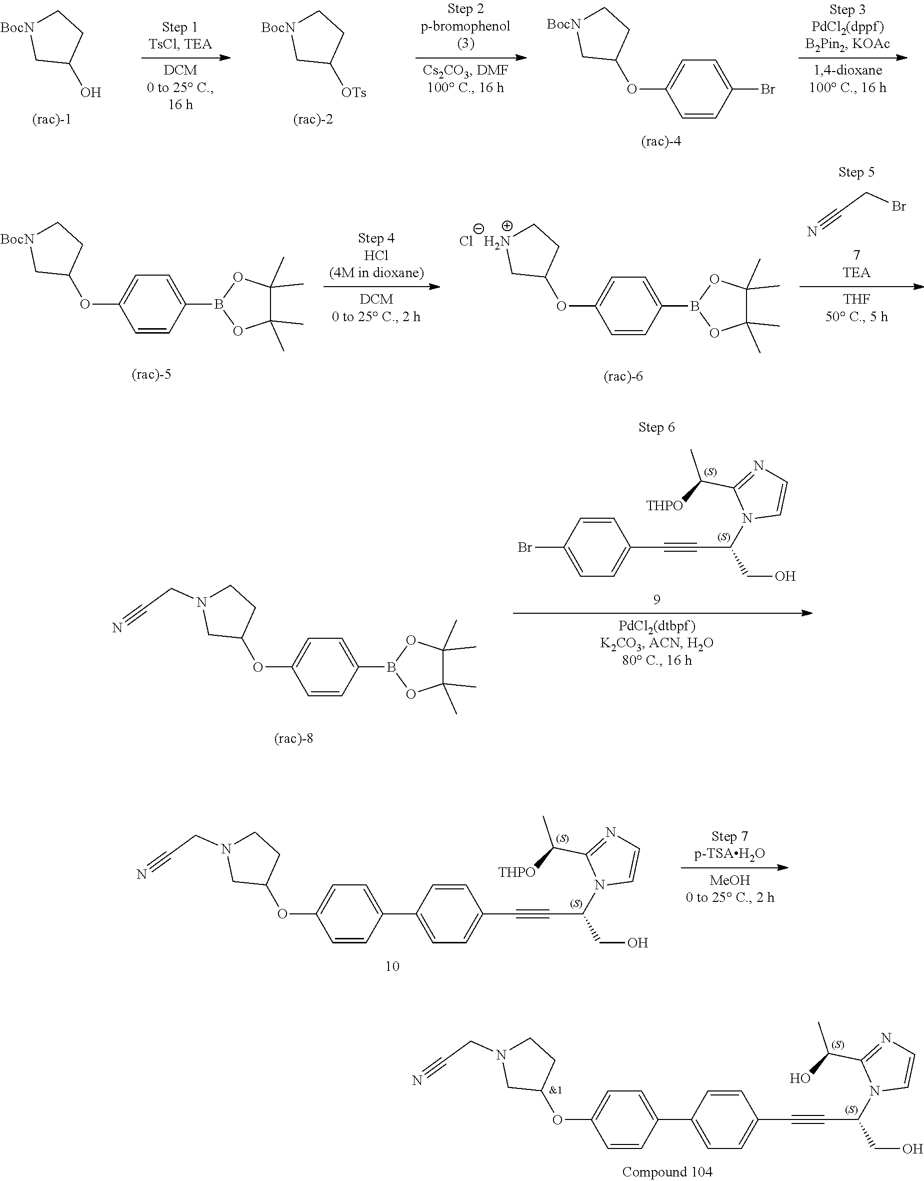

### Step 1:

To a stirred solution of (rac)-tert-butyl 3-hydroxypyrrolidine-1-carboxylate (1, 10 g, 53.4 mmol) in DCM (100 mL), were added TEA (51.1 mL, 374 mmol) and tosyl chloride (12.22 g, 64.1 mmol) at 0° C. The reaction mixture was stirred at 25° C. for 16 h. The reaction was quenched with water (100 mL) and extracted with DCM (100 mL×2). The combined organic layer was washed with brine (100 mL), dried over anhydrous Na₂SO₄, filtered and concentrated under reduced pressure to obtain (rac)-2 as an off-white solid.

Yield: 11.9 g (58%). LC-MS: Calculated for C16H23NO5S is 341.422 observed: 286.2 [M-t-Bu+1]⁺

### Step 2:

To a stirred solution of p-bromophenol (3, 5 g, 28.9 mmol) in DMF (150 mL), were added caesium carbonate (28.2 g, 87 mmol) and (rac)-2 (11.84 g, 34.7 mmol) at room temperature. The reaction mixture was stirred at 100° C. for 16 h. The reaction was quenched with cold water (500 mL) and extracted with EtOAc (150 mL×2). The combined organic layer was washed with brine (100 mL), dried over anhydrous Na₂SO₄, filtered and concentrated under reduced pressure. The crude residue thus obtained, was purified by using MPLC (manually packed cartridge, SiO₂ 230-400 mesh; 30% EtOAc in hexanes) to afford (rac)-4 as an off-white solid.

Yield: 8.2 g (79%). LC-MS: Calculated for C15H20BrNO3 is 342.23 Observed: 286.1 [M-t-Bu]⁺ and 288.1 [M-t-Bu+2]⁺

### Step 3:

To a stirred solution of (rac)-4 (5 g, 14.61 mmol) in 1,4-dioxane (100 mL), were added bis(pinacolato)diboron (5.57 g, 21.91 mmol) and potassium acetate (4.30 g, 43.8 mmol) at room temperature. The reaction mixture was degassed for 5 min, following which PdCl2(dppf) (1.069 g, 1.461 mmol) was added. The mixture was degassed with nitrogen for 2 min and then stirred at 100° C. for 16 h. The reaction mixture was quenched with water (100 mL) and extracted with EtOAc (100 mL×2). The combined organic layer was washed with brine (30 mL), dried over anhydrous Na₂SO₄, filtered and concentrated under reduced pressure. The crude residue was purified by using MPLC (manually packed SiO₂ cartridge, 230-400 mesh; 30% EtOAc in hexanes) to afford (rac)-5 as a pale-yellow liquid.

Yield: 4.8 g (67%). LC-MS: Calculated for C21H32BNO5 is 389.30 Observed: 334.1 [M-t-Bu+1]⁺

### Step 4:

To a stirred solution of (rac)-5 (3 g, 6.14 mmol) in anhydrous DCM (30 mL), was added hydrochloric acid (4 M in dioxane, 15.35 mL, 61.4 mmol) at 0° C. and the resulting mixture stirred at 25° C. for 2 h. The reaction mixture was then concentrated under reduced pressure, and the crude mass triturated with MTBE (20 mL) to obtain (rac)-6 as an off-white solid. The obtained solid showed 81% purity by LCMS. This crude product was taken to the next step without any further purification.

Yield: 1.7 g (69%)

LC-MS: Calculated for C16H25lNO3⁺ is 290.19 (Free base) Observed: 290.2 [M]⁺

### Step 5:

To a stirred solution of (rac)-6 (1.7 g, 5.88 mmol) in anhydrous THF (10 mL), were added TEA (2.458 mL, 17.64 mmol) and Bromoacetonitrile (7, 1.205 mL, 11.76 mmol) at 25° C. The reaction mixture was then stirred at 50° C. for 5 h. The reaction was quenched with water (50 mL) and extracted with EtOAc (20 mL×2). The combined organic layer was washed with brine (20 mL), dried over anhydrous Na₂SO₄, filtered and concentrated under reduced pressure to obtain compound 8 as an off-white solid.

Yield: 1.78 g (80%)

LC-MS: Calculated for C18H25BN2O3 is 328.22 Observed: 329.2 [M+1]⁺

### Step 6:

To a stirred solution of 8 (282 mg, 0.859 mmol) in acetonitrile (4 mL) and water (2 mL), were added 9 (300 mg, 0.715 mmol) and K2CO3 (297 mg, 2.146 mmol) at room temperature. The reaction mixture was purged for 5 min. To this reaction mixture, PdCl2(dtbpf) (23.31 mg, 0.036 mmol) was added and the purging continued for another 2 min. The reaction mixture was stirred for at 80° C. for 16 h. The reaction mixture was quenched with water (15 mL) and extracted with 20% MeOH in DCM (15 mL×2). The combined organic layer was washed with brine (15 mL), dried over anhydrous Na₂SO₄, filtered and concentrated under reduced pressure. The crude residue thus obtained, was purified by using MPLC (manually packed SiO₂ cartridge, 230-400 mesh; 3% MeOH in DCM) to afford 10 as a pale-yellow liquid.

Yield: 300 mg (69%)

UPLC-MS: Calculated for C₃₂H₃₆N₄O₄ is 540.66 Observed: 541.2 [M+1]⁺

### Step 7:

To a stirred solution of 10 (296 mg, 0.548 mmol) in MeOH (5 mL), was added p-toluenesulfonic acid monohydrate (312 mg, 1.643 mmol) at 0° C. The resulting reaction mixture was stirred at 25° C. for 2 h. The reaction mixture was concentrated and basified with sat. NaHCO₃ solution (10 mL). The aqueous layer was extracted with 20% MeOH in DCM (10 mL×2). The combined organic extract was dried over anhydrous Na₂SO₄, filtered and concentrated. The crude residue was purified by reverse-phase preparative HPLC (10 mM ammonium bicarbonate and acetonitrile) to obtain Compound 104 as a white solid.

Yield: 110 mg (43%)

UPLC-MS: Calculated for C₂₇H28N403 is 456.546 Observed:457.4 [M+1]⁺

1H-NMR (400 MHz, DMSO-d6): δ 7.65 (app t, J=8.80 Hz, 4H), 7.51 (d, J=8.40 Hz, 2H), 7.36 (app d, J=1.20 Hz, 1H), 7.00 (d, J=8.80 Hz, 2H), 6.84 (app d, J=0.80 Hz, 1H), 5.69 (t, J=6.00 Hz, 1H), 5.53 (t, J=6.00 Hz, 1H, exchanges with D2O), 5.37 (d, J=5.60 Hz, 1H, exchanges with D2O), 5.01-5.00 (m, 1H), 5.00-4.94 (m, 1H), 3.88-3.85 (m, 4H), 2.99-2.95 (m, 1H), 2.84-2.80 (m, 1H), 2.76 (dd, J=2.00, 10.40 Hz, 1H), 2.57-2.51 (m, 1H, merges with solvent peak), 2.38-2.33 (m, 1H), 1.88-1.84 (m, 1H), 1.51 (d, J=6.40 Hz, 3H). The final compound is a mixture of diastereomers; racemic at tail part (l-Amylose A_0.5% IPAm in MeOH tR=8.33 and 11.13 min.

### Example A55: Synthesis of Compound 105

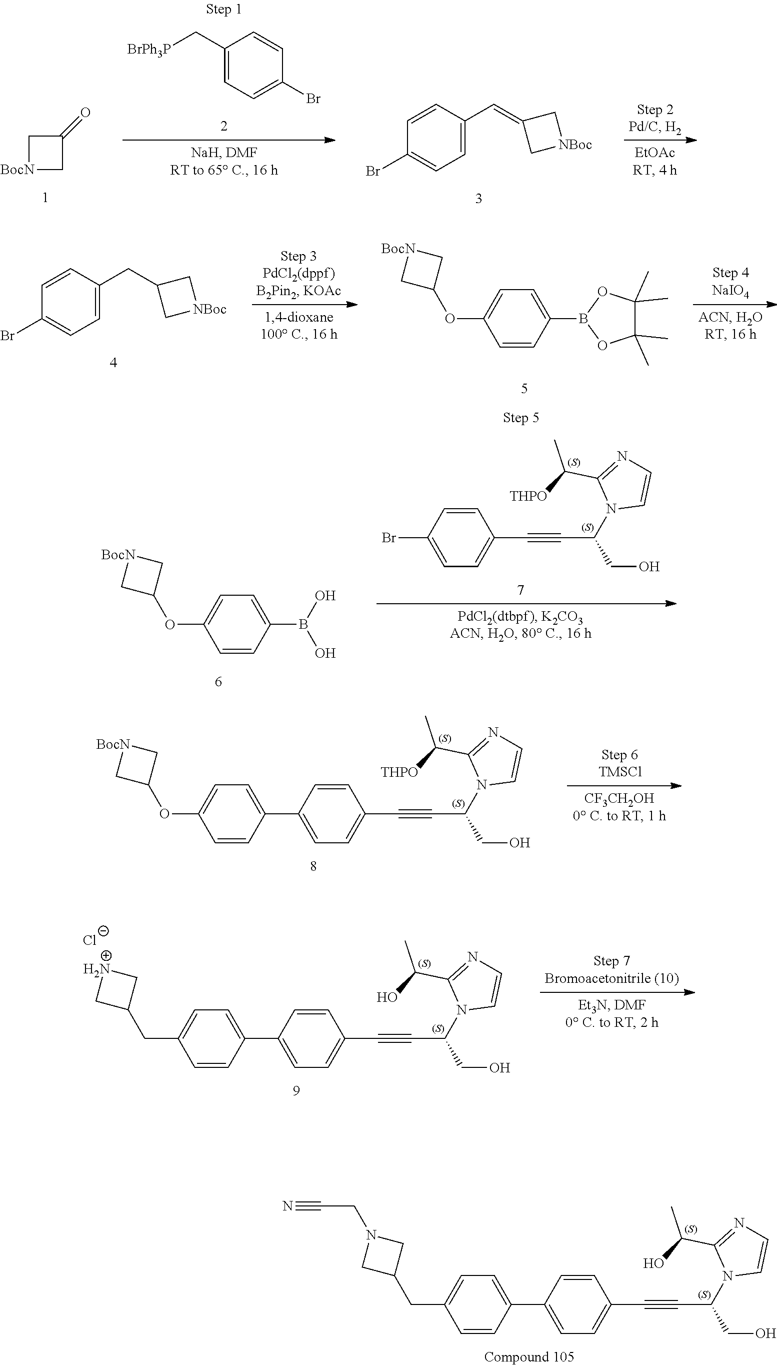

### Step 1:

To a solution of (4-bromobenzyl)triphenylphosphonium bromide (2, 16.46 g, 32.1 mmol) in DMF (280 mL), was added 60% sodium hydride (1.285 g, 32.1 mmol) portion wise at 0° C. After 15 min, a solution of 1-Boc-3-azetidinone (1, 5 g, 29.2 mmol) in DMF (80 mL) was added via addition funnel and the reaction mixture heated at 65° C. for 16 h. The reaction mixture was cooled to room temperature and quenched with aqueous ammonium chloride solution (1000 mL), extracted with EtOAc (2×200 mL). The combined organic layer was washed with brine solution (100 mL), dried over sodium sulfate and concentrated under reduced pressure. The resulting crude was purified by using MPLC (manually packed SiO₂ cartridge; 60-120 mesh size; 10-15% EtOAc in hexane) to obtain 3 as a white solid.

Yield=4.7 g (48%)

LC-MS: Calculated for C₁₅H₁₈BrNO₂ is 324.2, Observed: 224.0 [M-BOC]⁺ and 226.0 [(M-BOC)+2]⁺

One more batch was carried out on 2.5 g of 1 to get 2.7 g of 3.

### Step 2:

To a stirred solution of 3 (5 g, 15.42 mmol) in EtOAc (50 mL), was added 10% Pd—C (1.641 g, 1.542 mmol) and the resulting reaction mixture stirred at room temperature for 4 h under a blanket of hydrogen. One more batch was carried out on 2.2 g of 3. After completion, both batches mixed for work up. The reaction mixture was filtered through the Celite pad and washed with EtOAc (50 mL). The combined filtrate was concentrated to obtain 4 as a white solid.

Yield=5.1 g (combined yield for two batches)

LC-MS: Calculated for C₁₅H₂₀BrNO₂ is 326.23, Observed: 325.4 [M−1]⁻

### Step 3:

To a stirred solution of 4 (5.1 g, 15.63 mmol) in 1,4 dioxane (50 mL), were added potassium acetate (4.60 g, 46.9 mmol) and bis(pinacolato)diboron (5.95 g, 23.45 mmol) at room temperature and the resulting mixture purged with nitrogen for 10 min. To this reaction mixture, PdCl₂(dppf) (1.144 g, 1.563 mmol) was added and the purging with nitrogen continued for another 10 min. The resulting reaction mixture was stirred at 100° C. for 16 h. The reaction mixture was cooled to room temperature and quenched with water (50 mL) and extracted using EtOAc (50 mL×2). The combined organic layer was concentrated under reduced pressure. The resulting crude was purified by using MPLC (manually packed cartridge; SiO₂, 100-200 mesh size; 10% MeOH in DCM) to afford 5 as a white solid.

Yield: 5 g (67%)

LC-MS: Calculated for C₂₁H₃₂BNO₄ is 373.3, Observed: 274.2 [(M-BOC)+1]⁺

### Step-4:

To a stirred solution of 5 (3.0 g, 8.04 mmol) in a mixture of acetone (120 mL) and water (60.mL) was added ammonium acetate (1.858 g, 24.11 mmol) followed by sodium periodate (5.16 g, 24.11 mmol) at room temperature and the reaction mixture stirred at room temperature for 16 h. After the reaction mixture was concentrated, water (50 mL) was added to the residue, and extracted with EtOAc (2×40 mL). The combined organic layer was washed with brine (20 mL), dried over anhydrous Na₂SO₄, filtered, concentrated under reduced pressure to get 6 as a colorless gum.

Yield: 2.4 g (78%)

LC-MS: Calculated for C₁₅H₂₂BNO₄ is 291.1, Observed: 192.2 [(M-Boc)+1]⁺

### Step 5:

To a stirred solution of 7 (500 mg, 1.192 mmol) in acetonitrile (5 mL) and water (0.5 mL), were added 6 (521 mg, 1.789 mmol) and K₂CO₃ (494 mg, 3.58 mmol) at room temperature. The reaction mixture was purged with nitrogen for 5 min. To this reaction mixture, PdCl₂(dtbpf) (78 mg, 0.119 mmol) was added and the purging with nitrogen was continued for another 2 min. The reaction mixture was then stirred at 80° C. for 16 h. The reaction mixture was quenched with water (20 mL) and extracted with EtOAc (2×20 mL). The combined organic layer was washed with brine solution (10 mL), dried over anhydrous Na₂SO₄, filtered and concentrated under reduced pressure. The resulting crude mass was purified by MPLC (manually packed SiO₂ cartridge, 100-200 mesh size; 10% MeOH in DCM) to obtain 8 as a brown gum (LCMS showed 75% purity;). The product was taken to the next step without further purification.

Yield=410 mg (44%)

LC-MS: Calculated for C₃₅H₄₃N₃O₅ is 585.7, Observed: 586.4 [M+1]⁺

One more batch was carried out using 300 mg of 7 to get 300 mg of 8.

### Step 6:

To a stirred solution of 8 (0.5 g, 0.854 mmol) in trifluoroethanol (10 mL), was added trimethylsilyl chloride (0.174 mL, 1.280 mmol) at 0-5° C. under nitrogen. The reaction mixture was warmed to room temperature and stirred for 1 h. The reaction mixture was then concentrated under reduced pressure to obtain a crude mass which was co-distilled with hexane (2×10 mL) and dried to obtain 9 as a brown gum. (LCMS showed 55% purity;). The product was taken to the next step without further purification.

Yield=510 mg (crude)

LC-MS: Calculated for C25H28N3O2⁺ is 402.52, Observed: 402.3 [M]⁺

### Step 7:

To a stirred solution of 9 (510 mg, 1.27 mmol) in DMF (5 mL), was added triethylamine (1.042 mL, 7.62 mmol) at 0° C. under nitrogen and the reaction mixture stirred for 10 min. To the reaction mixture, Bromoacetonitrile (10, 0.134 mL, 1.905 mmol) was added dropwise. After complete addition, the reaction mixture was warmed to room temperature and stirred for 2 h. The reaction mixture was quenched with water (20 mL) and extracted with DCM (2×20 mL). The combined organic layer was concentrated under reduced pressure. The resulting crude mass, thus obtained, was purified by reverse phase preparative HPLC (Column: Shimpack C₁₈ (150*20 mm) 5 μm; Eluents: 10 mM ammonium bicarbonate in water and acetonitrile) to obtain Compound 105 as a white solid.

Yield=65 mg (11%)

LC-MS: Calculated for C₂₇H₂₈N₄O₂ is 440.5, Observed: 441.0 [M+1]⁺

1H-NMR (400 MHz, DMSO-d6): δ 7.68 (d, J=8.40 Hz, 2H), 7.62 (d, J=8.00 Hz, 2H), 7.54 (d, J=8.40 Hz, 2H), 7.36 (app d, J=1.20 Hz, 1H), 7.28 (app d, J=8.00 Hz, 2H), 6.84 (d, J=1.20 Hz, 1H), 5.69 (t, J=6.00 Hz, 1H), 5.53 (t, J=5.60 Hz, 1H, exchanges with D2O), 5.37 (d, J=5.60 Hz, 1H, exchanges with D2O), 4.97-4.91 (m, 1H), 3.87 (t, J=6.00 Hz, 2H), 3.60 (s, 2H), 3.38 (t, J=7.20 Hz, 2H), 3.01 (t, J=7.20 Hz, 2H), 2.88-2.86 (m, 2H), 2.73-2.67 (m, 1H), 1.51 (d, J=6.40 Hz, 3H).

SFC: 96.6%; tR=6.40 min (Column: LUX Amylose-1; Eluents: CO2 and 0.5% isopropyl amine in ACN and MeOH). Single isomer with SFC purity 96.6%

### Example A56: Synthesis of Compound 106

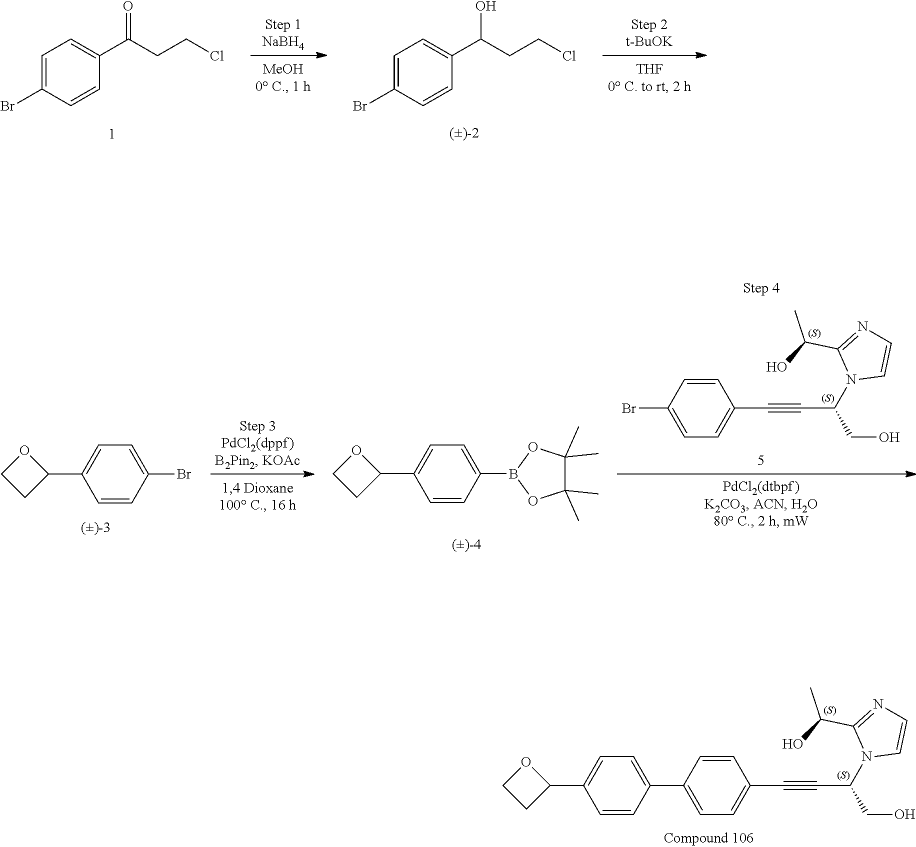

### Step 1

To a stirred solution of 1-(4-bromophenyl)-3-chloropropan-1-one (1, 10 g, 40.4 mmol) in MeOH (200 mL), was added sodium borohydride (3.06 g, 81 mmol) portion wise at 0° C. The reaction mixture was stirred at 0° C. for 1 h. The reaction was quenched with the addition of saturated ammonium chloride solution (200 mL) and extracted with EtOAc (100 mL×3). The combined organic layer was washed with brine (50 mL), dried over anhydrous Na₂SO₄, filtered and concentrated under reduced pressure to obtain (±)-2 as a colorless liquid.

Yield: 10 g (91%)

### Step 2:

To a stirred solution of (±)-2 (9 g, 36.1 mmol) in THE (150 mL), was added potassium tert-butoxide (12.14 g, 108 mmol) in portion wise manner at 0° C. and the resulting reaction mixture stirred at room temperature for 2 h. The reaction was quenched with saturated ammonium chloride solution (100 mL) and extracted with EtOAc (100 mL×3). The combined organic layer was washed with brine (50 mL), dried over anhydrous Na₂SO₄, filtered and concentrated under reduced pressure. The crude residue thus obtained, was purified by using MPLC (SiO₂ 230-400 mesh; 7% EtOAc in hexanes) to afford (±)-3 as a pale liquid.

Yield: 4.7 g (54%)

### Step 3:

To a stirred solution of (±)-3 (5.0 g, 23.47 mmol) in 1,4-dioxane (60 mL), were added bis(pinacolato)diboron (8.94 g, 35.2 mmol) and potassium acetate (6.91 g, 70.4 mmol) at room temperature. The reaction mixture was purged for 5 min with nitrogen. To this reaction mixture, PdCl2(dppf) (1.717 g, 2.347 mmol) was added and the purging continued for 2 min after which the reaction mixture was stirred at 100° C. for 16 b. The reaction mixture was quenched with water (200 mL) and extracted with EtOAc (100 mL×2). The combined organic layer was washed with brine (50 mL), dried over anhydrous Na₂SO₄, filtered and concentrated under reduced pressure. The crude residue thus obtained, was purified by using MPLC (SiO₂ 230-400 mesh; 17% EtOAc in hexanes) to afford (±)-4 as a pale-yellow liquid.

Yield: 4 g (54%)

### Step 4:

To a stirred solution of 4 (0.233 g, 0.895 mmol) in ACN (3 mL) and water (3 mL), were added 5 (0.2 g, 0.597 mmol) and K₂CO₃ (0.247 g, 1.790 mmol) at room temperature. The reaction mixture was purged for 5 min with nitrogen. To this reaction mixture, PdCl2(dtbpf) (0.039 g, 0.060 mmol) was added and the purging continued for 2 min. The reaction mixture was irradiated at 80° C. for 2 h in microwave reactor. The reaction mixture was quenched with water (10 mL) and extracted with 10% MeOH in DCM (10 mL×2). The combined organic layer was washed with brine (5 mL), dried over anhydrous Na₂SO₄, filtered and concentrated under reduced pressure. The crude residue, thus obtained, was purified by preparative HPLC (Column: X-BRIDGE C18 (19×250 mm) 5 μm; Eluents:10 mM ammonium bicarbonate in water and ACN) to obtain Compound 106 as a white solid.

Yield: 36 mg (15%)

LC-MS: Calculated for C24H24N2O3 is 388.4 Observed: 389.3 [M+1]⁺

1H-NMR (400 MHz, DMSO-d6): δ 7.74-7.71 (m, 4H), 7.57-7.53 (m, 4H), 7.36 (d, J=1.20 Hz, 1H), 6.85 (d, J=0.80 Hz, 1H), 5.78 (t, J=7.60 Hz, 1H), 5.70 (t, J=6.00 Hz, 1H), 5.54 (t, J=5.60 Hz, 1H, exchanges with D2O), 5.38 (d, J=5.60 Hz, 1H, exchanges with D2O), 4.94 (t, J=6.00 Hz, 1H), 4.70 (t, J=5.60 Hz, 1H), 4.60-4.55 (m, 1H), 3.88 (t, J=6.00 Hz, 2H), 3.03-2.99 (m, 1H), 2.61-2.56 (m, 1H), 1.51 (d, J=6.40 Hz, 3H). Mixture of diastereomers; racemic at tail (R,R-Whelk_0.5% IPAm in MeOH; tR=4.10 and 4.74 min).

### Example A57: Synthesis of Compound 107

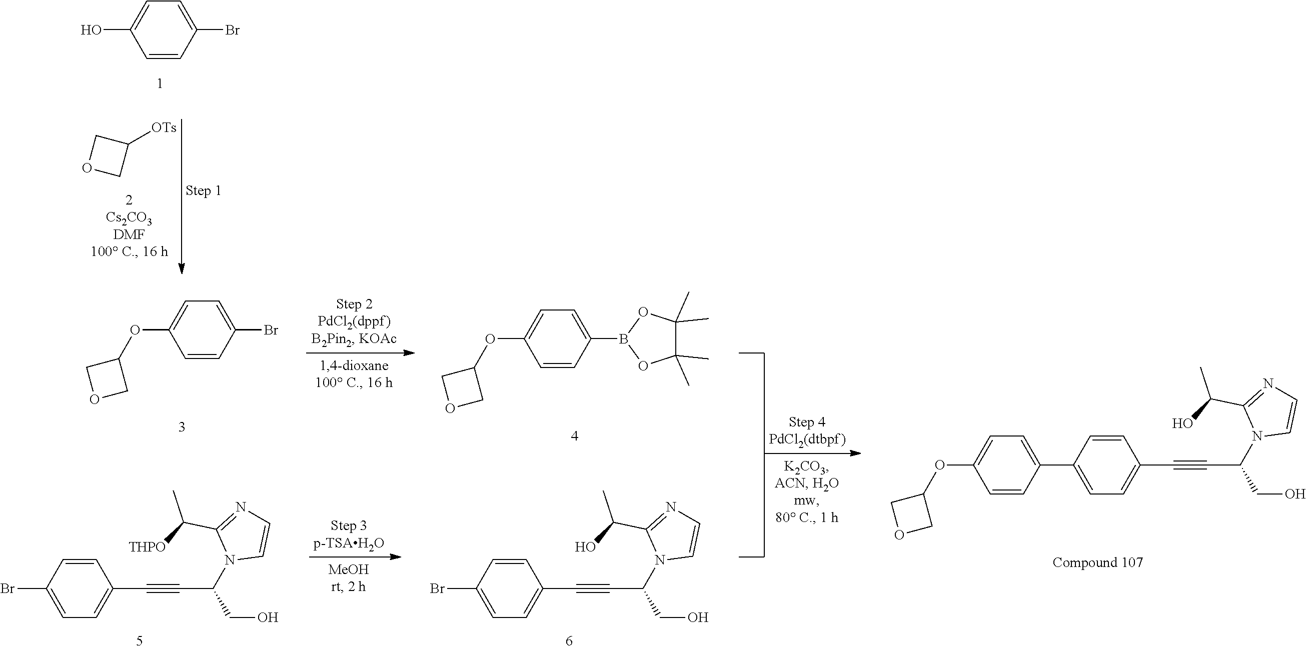

### Step 1:

To a stirred solution of 4-bromophenol (1, 6.67 g, 38.6 mmol) in DMF (80 mL), were added oxetan-3-yl 4-methylbenzenesulfonate (2, 8 g, 35.0 mmol) and cesium carbonate (34.3 g, 105 mmol) at room temperature. The reaction mixture was stirred at 100° C. for 16 h. The reaction mixture was quenched with water (300 mL) and extracted with MTBE (200 mL×4). The combined organic layers were washed with brine (20 mL), dried over anhydrous Na₂SO₄, filtered and concentrated under reduced pressure. The crude residue was purified by MPLC (manually packed cartridge, SiO₂ 230-400 mesh; 15% EtOAc in hexanes) to obtain 3 as a white solid.

Yield: 7.5 g (84%)

### Step 2:

To a stirred solution of 3 (4.0 g, 17.46 mmol) in 1,4-dioxane (60 mL), were added bis(pinacolato)diboron (6.65 g, 26.2 mmol) and potassium acetate (5.14 g, 52.4 mmol) and the reaction mixture degassed for 5 min. To this mixture, PdCl2(dppf) (1.278 g, 1.746 mmol) was added and the purging continued for another 2 min. The resulting reaction mixture was stirred at 100° C. for 16 h. The reaction mixture was quenched with water (100 mL) and extracted with EtOAc (100 mL×2). The combined organic layer was washed with brine (50 mL), dried over anhydrous Na₂SO₄, filtered and concentrated under reduced pressure. The crude residue was purified by MPLC (manually packed cartridge, SiO₂ 230-400 mesh; 20% EtOAc in hexanes) to afford 4 as a pale-yellow solid.

Yield: 3.5 mg (66%)

### Step 3:

To a solution of 5 (0.3 g, 0.715 mmol) in MeOH (5 mL), was added p-toluenesulfonic acid monohydrate (0.408 g, 2.146 mmol) at 0° C. The resulting reaction mixture was stirred at room temperature for 2 h. The volatiles were concentrated under reduced pressure, and the resulting crude residue basified with sat. NaHCO3 solution (15 mL). The aqueous layer was extracted with 10% MeOH in DCM (15 mL×2). The combined organic extract was dried over anhydrous Na₂SO₄, filtered and concentrated to afford 6 as a brown solid.

Yield: 220 mg (90%)

LC-MS: Calculated for C15H15BrN2O2 is 335.20, Observed: 335.0 [M]⁺ and 337.0 [M+2]⁺

### Step 4:

To a stirred solution of 6 (170 mg, 0.507 mmol) in acetonitrile (5 mL) and water (5 mL), were added 4 (210 mg, 0.761 mmol) and K2CO3 (210 mg, 1.521 mmol) at room temperature. The resulting mixture was purged with nitrogen for 5 min. To this reaction mixture, PdCl2(dtbpf) (33.1 mg, 0.051 mmol) was added and the purging continued for another 2 min. The resulting reaction was irradiated at 80° C. for 1 h in a microwave reactor. The reaction mixture was monitored by TLC (5% MeOH in DCM). The reaction was quenched with water (2 mL) and extracted with 10% MeOH in DCM (5 mL×3). The combined organic layer was washed with brine (5 mL), dried over anhydrous Na₂SO₄, filtered and concentrated under reduced pressure. The crude residue was purified by preparative HPLC (Column: Shimpack-C18 (150×20 mm) 5 μm; Eluents: 10 mM ammonium bicarbonate in water and ACN) to obtain Compound 107 as an off-white solid

Yield: 40 mg (19%)

UPLC-MS: Calculated for C24H24N2O4 is 404.47, Observed: 405.4 [M+1]⁺. The final compound is single isomer

### Example A58: Synthesis of Compound 108

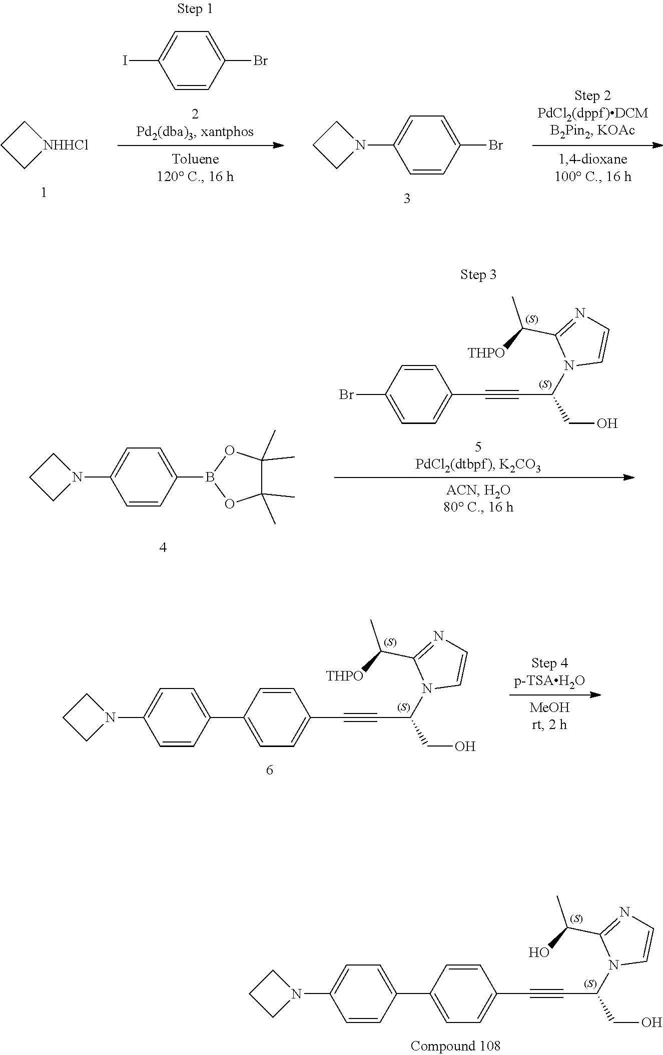

### Step 1:

To a stirred solution of 1-bromo-4-iodobenzene (2, 10 g, 35.3 mmol) in toluene (200 mL), were added azetidine hydrochloride (1, 3.31 g, 35.3 mmol), Cs₂CO₃ (46.1 g, 141 mmol) and xantphos (1.227 g, 2.121 mmol) at room temperature, and the mixture degassed with nitrogen for 5 min. To this reaction mixture, Pd₂(dba)₃ (0.647 g, 0.707 mmol) was added and the degassing continued for another 2 min. The resulting reaction mixture was stirred at 120° C. for 16 h. The pad was further washed with EtOAc (200 mL) and the combined filtrate concentrated under reduced pressure. The resulting crude residue was purified by MPLC (manually packed cartridge, SiO₂ 100-200 mesh; 10% EtOAc in hexanes) to afford 3 as a yellow solid.

Yield=4 g (43.2%)

LC-MS: Calculated for C9H10BrN is 212.09, Observed: 212.0 [M]⁺ and 214.0 [M+2]⁺

### Step 2:

To a solution of 3 (4 g, 18.86 mmol) in 1,4-dioxane (50 mL), were added potassium acetate (7.82 g, 56.6 mmol) and bis(pinacolato)diboron (5.75 g, 22.3 mmol) at room temperature. The resulting mixture was purged with nitrogen for 5 min. To this mixture, PdCl₂(dppf)·DCM adduct (1.54 g, 1.886 mmol) was added and the degassing continued for another 2 min. The reaction mixture was heated at 100° C. for 16 h. After completion of the reaction, the inorganic solids were filtered through a Celite pad and washed with EtOAc (100 mL). The filtrate was concentrated under reduced pressure to afford a crude residue which was purified by MPLC (manually packed cartridge, SiO₂ 100-200 mesh size; 20% EtOAc in hexanes) to afford 4 as a yellow solid.

Yield: 2.2 g (38%)

LC-MS: Calculated for C15H22BNO2 is 259.156, Observed: 260.2 [M+1]+

### Step-3:

To a stirred solution of 5 (2 g, 4.77 mmol) in ACN (30 mL) and water (3 mL), were added 4 (1.483 g, 5.72 mmol) and K₂CO₃ (1.978 g, 14.31 mmol) at room temperature. The reaction mixture was purged with nitrogen for 5 min. To this reaction mixture, PdCl₂(dtbpf) (0.311 g, 0.477 mmol) was added and purging continued for another 2 min. The resulting reaction mixture was stirred at 80° C. for 16 h. The reaction mixture was quenched with water (30 mL), extracted with 10% MeOH in DCM (2×40 mL). The combined organic lay er was washed with brine solution (30 mL), dried over anhydrous Na₂SO₄, filtered and concentrated under reduced pressure. The resulting crude product was purified by MPLC (manually packed SiO₂ cartridge, 100-200 mesh size; 5% MeOH in DCM) to afford 6 as a pale-yellow solid.

Yield: 600 mg (23%)

LC-MS: Calculated for C29H33N3O3 is 471.60, Observed: 472.2 [M+1]⁺

### Step 4:

To a stirred solution of 6 (200 mg, 0.360 mmol) in MeOH (5 mL), was added p-toluenesulfonic acid monohydrate (205 mg, 1.080 mmol) at room temperature. The reaction mixture was stirred for 2 h. The volatiles were evaporated under reduced pressure. The resulting residue was diluted with 5% NaHCO3 solution (15 mL) and extracted with 10% MeOH in DCM (2×20 mL). The combined organic layers were dried over anhydrous Na₂SO₄, filtered and concentrated under reduced pressure. The crude mass was purified by reversed phase preparative HPLC (Column: Shimpack C18 (150*20 mm) 5 μm, Eluents: 10 mM ammonium bicarbonate in water and acetonitrile) to afford Compound 108 as an off-white solid.

Yield=37 mg (26.5%)

LC-MS: Calculated for C₂₄H₂₅N₃O₃ is 387.483, Observed: 388.4 [M+1]⁺

### Example A59: Synthesis of Compound 109

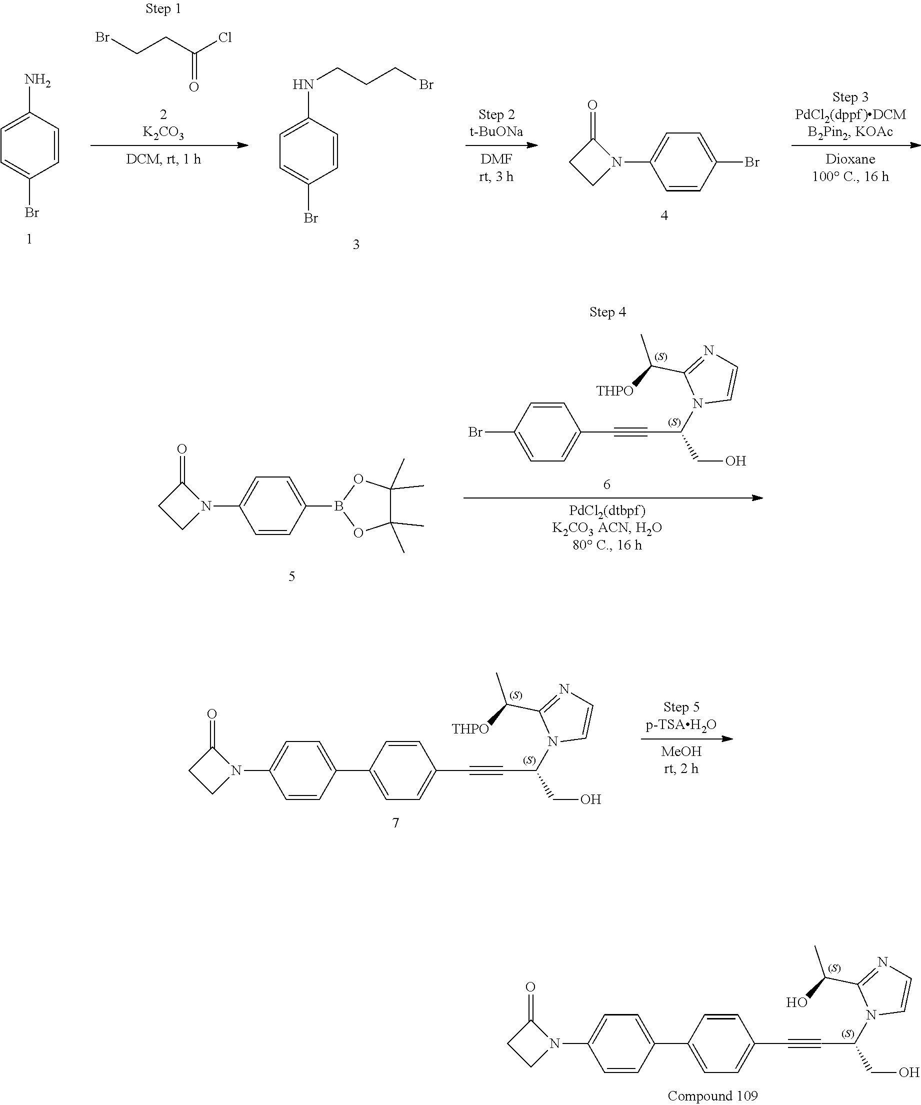

### Step 1:

To a stirred solution of 4-bromoaniline (1, 5.0 g, 29.1 mmol) and K₂CO₃ (8.23 g, 59.6 mmol) in DCM (100 mL), were added 3-bromopropanoyl chloride (2, 2.99 mL, 29.6 mmol) under nitrogen. The resulting reaction mixture was stirred at room temperature for 1 h. After completion of the reaction, the mixture was diluted with water (100 mL). The aqueous layer was separated and extracted with DCM (2×100 mL). The combined organic layer was dried over Na₂SO₄, filtered, and concentrated under reduced pressure to obtain 3 as an off-white solid.

Yield=9.1 g (97%)

LC-MS: Calculated for C₉H₉Br₂NO is 306.9, Observed: 305.0 [M]⁺, 308.0 [M+2]⁺ and 310.0 [M+4]⁺

### Step 2:

To a stirred solution of 3 (4.1 g, 13.36 mmol) in DMF (50 mL), was added sodium tert-butoxide (1.348 g, 14.02 mmol) under nitrogen. The resulting reaction mixture was stirred at room temperature for 3 h. After completion, the reaction was quenched with water (250 mL) and extracted with EtOAc (2×150 mL). The combined organic layer was washed with brine solution (100 mL), dried over Na₂SO₄, and concentrated under reduced pressure. The crude residue was purified by MPLC (manually packed SiO₂ cartridge, 100-200 mesh size; 30% EtOAc in hexanes) to afford crude as an off-white solid. The crude was stirred with 30% acetone in hexane (10 mL); obtained solid was filtered and washed with n-hexane (5 mL) to obtain to obtain N-(4-bromophenyl)acrylamide (450 mg, 1.805 mmol, 13.52% yield) as a white solid. The filtrate was concentrated to afford 1-(4-bromophenyl)azetidin-2-one (4, 300 mg, 1.205 mmol, 9.02% yield) as a white solid.

Yield=300 mg (9%)

LC-MS: Calculated for C₉H₈BrNO is 226.073, Observed: 226.0 [M]⁺ and 228.2 [M+2]⁺

### Step 3:

To a solution of 4 (300 mg, 1.327 mmol) in 1,4-dioxane (10 mL), were added potassium acetate (391 mg, 3.98 mmol) and bis(pinacolato)diboron (404 mg, 1.592 mmol) at room temperature. The resulting reaction mixture was purged with nitrogen for 5 min. To this reaction mixture, PdCl₂(dppf)·DCM adduct (108 mg, 0.133 mmol) was added and the purging continued for another 2 min. The reaction mixture was heated at 100° C. for 16 h. The inorganic solid was filtered off through Celite pad and washed with EtOAc (50 mL). The filtrate was concentrated under reduced pressure. The resulting crude residue was purified by MPLC (manually packed SiO₂ cartridge, 100-200 mesh size; 30% EtOAc in hexanes) to afford 5 as a yellow solid.

Yield: 235 mg (52%)

LC-MS: Calculated for C₁₅H₂₀BNO₃ is 273.1, Observed: 274.2 [M+1]⁺

### Step-4:

To a stirred solution of 6 (240 mg, 0.572 mmol) in ACN (5 mL) and water (0.5 mL), were added 5 (232 mg g, 0.687 mmol) and K₂CO₃ (237 mg, 1.717 mmol) at room temperature. The reaction mixture was purged with nitrogen for 5 min. To this reaction mixture, PdCl₂(dtbpf) (37.3 mg, 0.057 mmol) was added and the purging continued for another 2 min. The resulting reaction mixture was stirred at 80° C. for 16 h. The reaction mixture was concentrated under reduced pressure. To the obtained residue, water (10 mL) was added and extracted with 10% MeOH in DCM (2×10 mL). The combined organic layer was washed with brine solution (10 mL), dried over anhydrous Na₂SO₄, filtered, and concentrated under reduced pressure. The resulting crude product was purified by MPLC (manually packed SiO₂ cartridge, 100-200 mesh size; 5% MeOH in DCM) to afford 7 as a pale-yellow solid.

Yield: 150 mg (51%)

LC-MS: Calculated for C₂₉H₃₁N₃O₄ is 485.5, Observed: 486.4 [M+1]⁺

### Step 5:

To a stirred solution of 7 (110 mg, 0.227 mmol) in MeOH (5 mL), p-toluene sulfonic acid monohydrate (129 mg, 0.680 mmol) was added at room temperature and the reaction mixture stirred for 2 h. The volatiles were evaporated under reduced pressure and the resulting residue was diluted with water (10 mL) and extracted with 10% MeOH in DCM (2×20 mL). The combined organic layer was dried over anhydrous Na₂SO₄, filtered, and concentrated under reduced pressure. The crude mass was purified by reverse phase preparative HPLC (Column: Shimpack C18 (150*20 mm) 5 μm, Eluents: 10 mM ammonium bicarbonate in water and acetonitrile) to afford Compound 109 as an off-white solid.

Yield=23 mg (25%)

LC-MS: Calculated for C₂₄H₂₃N₃O₃ is 401.4, Observed: 402.0 [M+1]⁺

1H-NMR (400 MHz, DMSO-d6): δ 7.74 (d, J=8.40 Hz, 2H), 7.70 (d, J=8.40 Hz, 2H), 7.53 (d, J=8.40 Hz, 2H), 7.44 (d, J=8.80 Hz, 2H), 7.36 (app d, J=1.20 Hz, 1H), 6.84 (app d, J=1.20 Hz, 1H), 5.70 (t, J=6.00 Hz, 1H), 5.53 (t, J=5.60 Hz, 1H, exchanges with D2O), 5.37 (d, J=5.60 Hz, 1H, exchanges with D2O), 4.97-4.91 (m, 1H), 3.87 (t, J=6.00 Hz, 2H), 3.68 (t, J=4.40 Hz, 2H), 3.11 (t, J=4.40 Hz, 2H), 1.51 (d, J=6.40 Hz, 3H). Single isomer with SFC purity=100%.

### Example A60: Synthesis of Compound 110

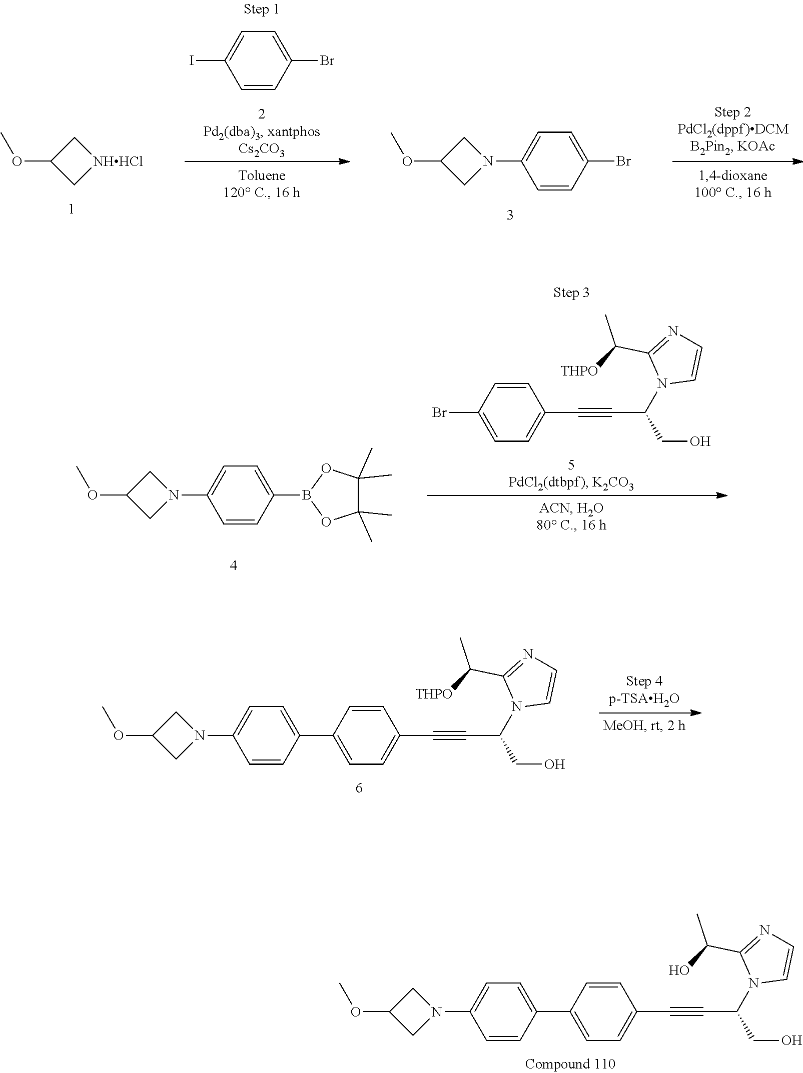

### Step 1:

To a stirred solution of 4-bromo-1-iodobenzene (2, 10 g, 35.3 mmol) in toluene (200 mL), were added 3-methoxyazetidine hydrochloride (1, 4.37 g, 35.3 mmol), Cs₂CO₃ (46.1 g 141 mmol) and xantphos (1.227 g, 2.121 mmol) at room temperature. The reaction mixture was degassed with nitrogen for 5 min. To this reaction mixture, Pd2(dba)3 (0.647 g, 0.707 mmol) was added and the degassing continued for another 2 min. The resulting reaction mixture was stirred at 120° C. for 16 h. After completion of the reaction, the inorganic solids were filtered through Celite pad. The pad was further washed with EtOAc (200 mL) and the combined filtrate concentrated under reduced pressure. The resulting crude residue was purified by MPLC (manually packed SiO₂ cartridge, 100-200 mesh; 20% EtOAc in hexanes) to afford 3 as a yellow solid.

Yield=8 g (76%)

LC-MS: Calculated for C₁₀H₁₂BrNO is 242.12, Observed: 242.0 [M]⁺ and 244.0 [M+2]⁺

### Step 2:

To a solution of 3 (8 g, 33 mmol) in 1,4-dioxane (100 mL), were added potassium acetate (9.73 g, 99.0 mmol) and bis(pinacolato)diboron (10.07 g, 39.7 mmol) at room temperature. The resulting mixture was purged with nitrogen for 5 min. To this mixture, PdCl₂(dppf)·DCM adduct (2.70 g, 3.30 mmol) was added and degassing continued for another 2 min. The reaction mixture was heated at 100° C. for 16 h. After completion of reaction, the inorganic solids were filtered through celite pad and washed with EtOAc (100 mL). The filtrate was concentrated under reduced pressure. The resulting crude residue was purified by MPLC (manually packed SiO₂ cartridge, 100-200 mesh size; 20% EtOAc in hexanes). The obtained residue was stirred with n-hexane (50 mL) and filtered to afford 4 as an off-white solid.

Yield: 4.0 g (41%)

LC-MS: Calculated for C₁₆H₂₄BNO₃ is 289.18, Observed: 290.4 [M+1]⁺

### Step-3:

To a stirred solution of 5 (2 g, 4.77 mmol) in ACN (20 mL) and water (2 mL), were added 4 (2.069 g, 7.15 mmol) and K₂CO₃ (1.978 g, 14.31 mmol) at room temperature. The reaction mixture was degassed with nitrogen for 5 min. To this reaction mixture, PdCl₂(dtbpf) (0.311 g, 0.477 mmol) was added, and the degassing continued for another 2 min. The resulting reaction mixture was stirred at 80° C. for 16 h. The reaction mixture was quenched with water (50 mL), and extracted with 10% MeOH in DCM (2×50 mL). The combined organic layer was washed with brine solution (30 mL), dried over anhydrous Na₂SO₄, filtered and concentrated under reduced pressure. The resulting crude product was purified by MPLC (manually packed SiO₂ cartridge, 100-200 mesh size; 5% MeOH in DCM) to afford 6 as a pale-yellow solid.

Yield: 1 g (39%)

LC-MS: Calculated for C₃₀H₃₅N₃O₄ is 501.63, Observed: 502.2 [M+1]⁺

### Step 4:

To a stirred solution of 6 (180 mg, 0.359 mmol) in MeOH (5 mL), was added p-toluenesulfonic acid monohydrate (205 mg, 1.076 mmol) at room temperature and the reaction mixture stirred for 2 h. The volatiles were evaporated under reduced pressure; the resulting residue was diluted with 5% NaHCO3 solution (15 mL) and extracted with 10% MeOH in DCM (2×20 mL). The combined organic layer was dried over anhydrous Na₂SO₄, filtered and concentrated under reduced pressure. The resulting crude mass was purified by reverse phase preparative HPLC (Column: Shimpack C18 (150*20 mm) 5 μm, Eluents: 10 mM ammonium bicarbonate in water and acetonitrile) to afford Compound 110 as an off-white solid.

Yield=65 mg (43%)

LC-MS: Calculated for C₂₅H₂₇N₃O₃ is 417.51, Observed: 418.2 [M+1]⁺

### Example A61: Synthesis of Compound 111

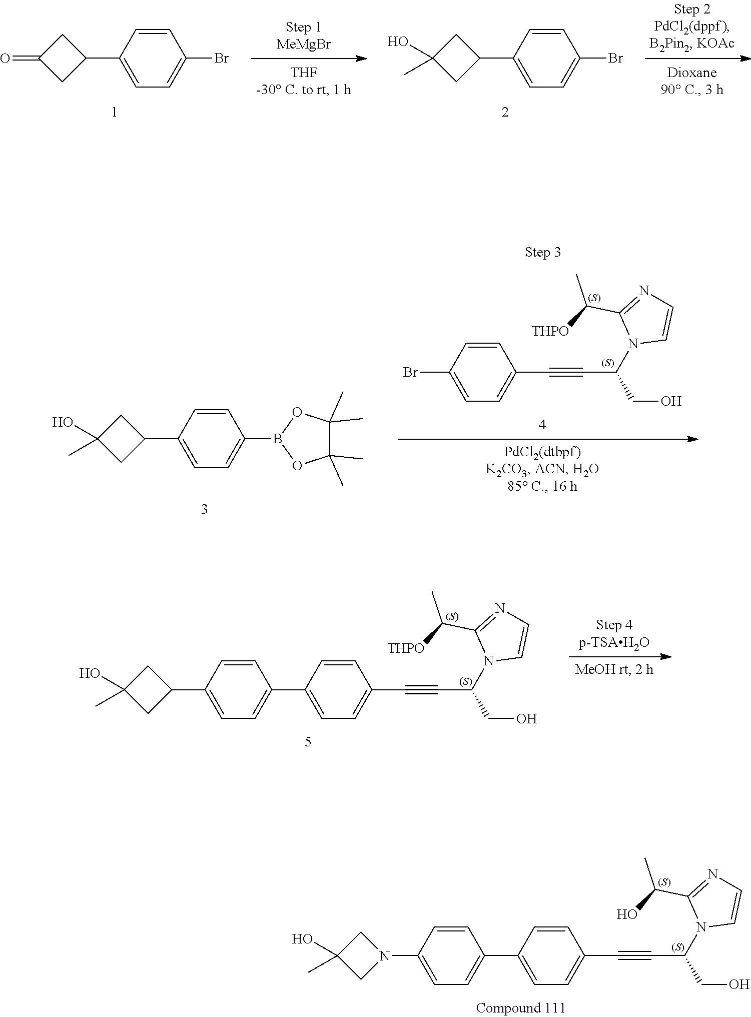

### Step 1:

To a stirred solution of 3-(4-bromophenyl)cyclobutan-1-one (1, 4.0 g, 17.77 mmol) in THF (50 mL), was added methylmagnesium bromide (1.0 M in THF; 39.1 mL, 39.1 mmol) dropwise at −30° C. After complete addition, the resulting reaction mixture was stirred for 15 min. then slowly warmed to room temperature and stirred for 1 h. The reaction mixture was cooled to 0° C. and quenched with sat. NH₄Cl solution (15 mL) and extracted with EtOAc (30 mL×2). The combined organic extract was washed with brine solution (15 mL), dried over anhydrous sodium sulphate, and concentrated under reduced pressure to afford crude residue. The crude residue was purified by MPLC (manually packed cartridge, SiO₂ 230-400 mesh size; 0-10% EtOAc in hexanes) to afford 2 as an off-white solid.

Yield: 1.7 g (35%)

### Step 2:

To the stirred solution of 2 (0.51 g, 2.115 mmol) in 1,4-dioxane (5 mL), were added bis(pinacolato)diboron (0.806 g, 3.17 mmol), potassium acetate (0.415 g, 4.23 mmol) at room temperature. The reaction mixture was purged with nitrogen for 5 min and PdCl₂(dppf) (0.155 g, 0.212 mmol) was added. The resulting reaction mixture was stirred at 90° C. for 3 h. The reaction mixture was cooled to room temperature and then filtered through Celite bed. The Celite bed was washed with EtOAc (100 mL×2) and the filtrate concentrated under reduced pressure to afford a crude residue. The crude residue was purified by MPLC (manually packed cartridge, SiO₂ 230-400 mesh size; 0-10% EtOAc in hexanes) to afford 3 as an off-white solid.

Yield: 0.4 g (59%)

### Step 3:

To the stirred solution of 4 (500 mg, 1.192 mmol) in acetonitrile (3 mL) and water (2 mL), were added boronate (3, 526 mg, 1.550 mmol), K₂CO₃ (330 mg, 2.385 mmol) at room temperature. The resulting reaction mixture was purged with nitrogen for 5 min. To this reaction mixture, PdCl₂(dtbpf) (117 mg, 0.179 mmol) was added at room temperature and the resulting reaction mixture stirred at 85° C. for 16 h. The reaction mixture was cooled to room temperature, diluted with water (10 mL) and extracted with 10% MeOH in DCM (25 mL×3). The combined organic layer was washed with brine solution (20 mL), filtered and concentrated under reduced pressure to afford a crude residue. The crude residue was purified by MPLC (manually packed cartridge, SiO₂ 230-400 mesh size; 30% MeOH in DCM) to afford 5 as a reddish solid.

Yield: 120 mg (19%)

LCMS: Calculated for C₃₁H₃₆N₂O₄ is 500.64. Observed: 501.2 [M+1]⁺

### Step 4:

To the stirred solution of 5 (130 mg, 0.260 mmol) in MeOH (30 mL), was added p-toluenesulfonic acid monohydrate (148 mg, 0.779 mmol) at 0° C. The resulting reaction mixture was allowed to stir at room temperature for 4 h. The reaction was quenched with sat. NaHCO₃ solution (10 mL) at 0° C. and extracted with DCM (100 mL×2). The combined organic extract was washed with brine (8 mL), dried over anhydrous sodium sulphate, filtered and concentrated to afford crude residue. Two more batches were carried out on 130 mg of 5 to get 210 mg of crude material. All three batches were mixed and purified by reversed phase preparative HPLC to afford Compound 111 as a white solid. Yield: 48 mg (combined yield for three batches). Two more batches were carried out separately on 500 mg and 120 mg scale of 5 to get 70 mg and 16 mg of Compound 111 respectively. All the batches were mixed and dissolved in acetonitrile (1 mL) and water (2 mL). The mixture was lyophilized to obtain 133 mg of Compound 111 as an off-white solid.

LCMS: Calculated for C₂₆H₂₈N₂O₃ is 416.52, Observed: 417.0 [M+1]⁺ The final compound was a mixture of diastereomers

### Example A62: Synthesis of Compound 112

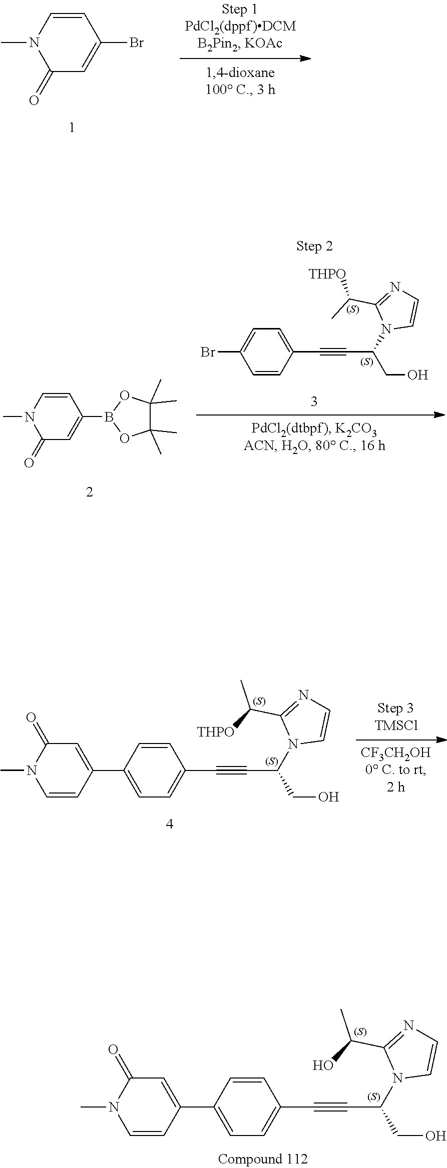

### Step-1:

To a solution of 4-bromo-1-methylpyridin-2(1H)-one (1, 3.0 g, 15.96 mmol) in dioxane (50 mL), were added potassium acetate (4.70 g, 47.90 mmol) and bis(pinacolato)diboron (6.08 g, 23.93 mmol) at room temperature. The resulting mixture was purged with nitrogen for 10 min. To this reaction mixture, PdCl2(dppf)·DCM adduct (1.303 g, 1.596 mmol) was added and the purging continued for 2 min. The reaction mixture was stirred at 100° C. for 3 h. The inorganic solids were filtered through a celite pad and washed with EtOAc (100 mL). The filtrate was concentrated under reduced pressure to afford 2 as a brown gum.

Yield: 4.0 g (83%)

### Step-2:

To a stirred solution of 3 (2.0 g, 4.770 mmol) in ACN (15 mL) and water (15 mL), were added boronate ester 2 (2.1 g, 7.15 mmol) and K2CO3 (1.97 g, 14.31 mmol) at room temperature. The reaction mixture was degassed with nitrogen for 5 min. To this reaction mixture, PdCl2(dtbpf) (0.311 g, 0.477 mmol) was added and degassing continued for another 2 min. The resulting reaction mixture was stirred at 80° C. for 16 h. The reaction mixture was quenched with water (50 mL), and extracted with 10% MeOH in DCM (50 mL×2). The combined organic layer was washed with brine solution (30 mL), dried over anhydrous Na2SO4, filtered and concentrated under reduced pressure. The resulting crude product was purified by MPLC (manually packed cartridge, SiO2 230-400 mesh size; 5% MeOH in DCM) to afford 4 as a brown solid. LCMS showed 66% purity; the product was taken to the next step.

Yield: 500 mg (15%)

LC-MS: Calculated for C26H29N3O4 is 447.54, Observed: 448.4 [M+1]⁺

### Step-3:

To a stirred solution of 4 (0.370 g, 0.546 mmol) in 2,2,2-trifluoroethanol (5 mL), was added chlorotrimethylsilane (0.159 mL, 1.240 mmol) at 0° C. The reaction mixture was stirred at room temperature for 2 h. The reaction mixture was concentrated under reduced pressure to afford a crude residue. The resulting crude residue was purified by reversed-phase preparative HPLC (Column: YMC C18 (250*20 mm) 5 μM; Eluents: 10 mM ammonium bicarbonate in water and ACN) to afford Compound 112 as a white solid.

Yield=45 mg (27%)

LC-MS: Calculated for C21H21N3O3 is 363.417, Observed: 364.2 [M+1]⁺

### Example A63: Synthesis of Compounds 113 and 114

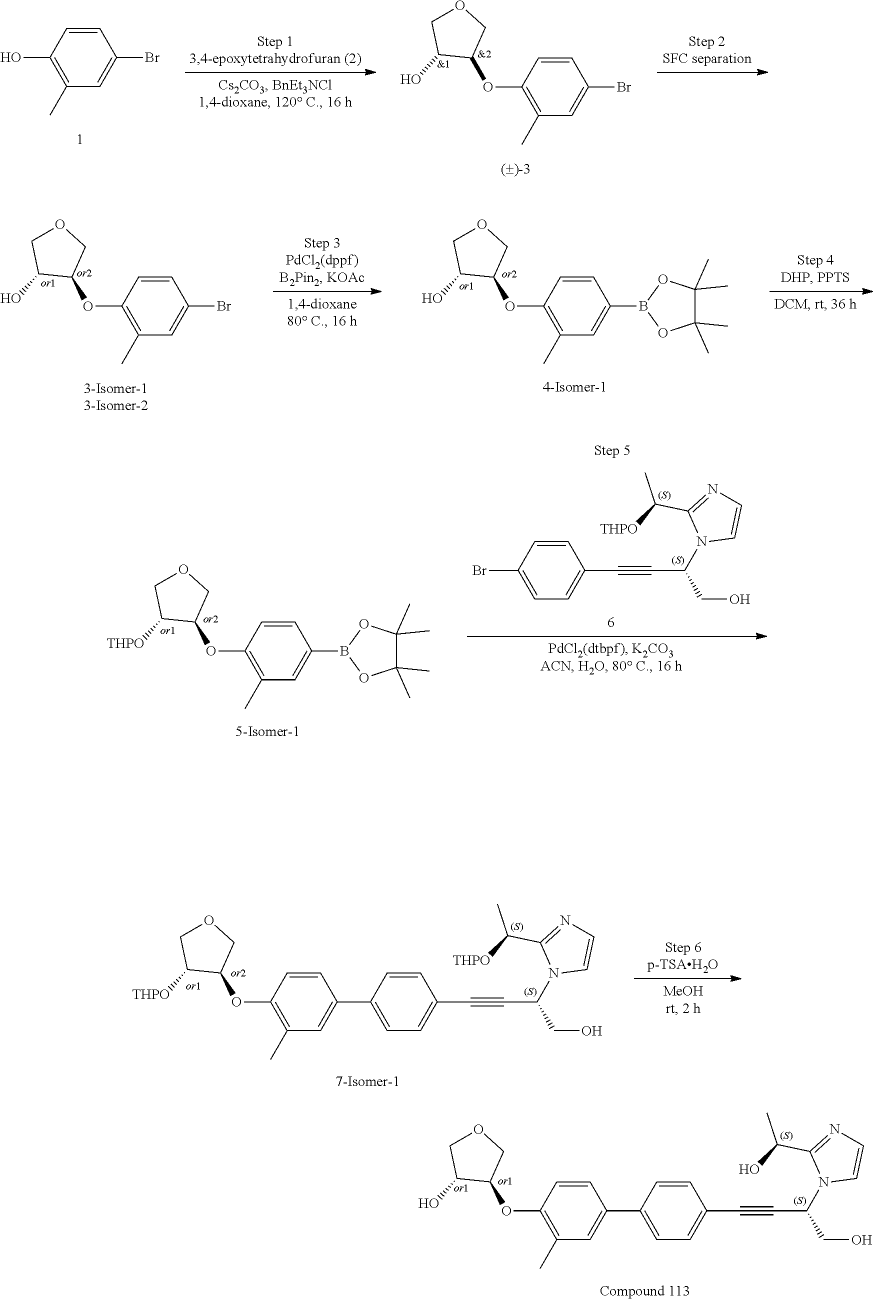

### Step 1:

To a solution of 4-bromo-2-methylphenol (1, 10 g, 53.5 mmol) in 1,4-dioxane (150 mL), were added cesium carbonate (26.1 g, 80 mmol), 3,4-epoxytetrahydrofuran (2, 6.90 g, 80 mmol) and benzyltriethylammonium chloride (2.436 g, 10.69 mmol) at room temperature, and the resulting reaction mixture stirred at 120° C. for 16 h. The reaction mixture was cooled to room temperature, filtered through a Celite bed. The bed was washed with EtOAc (2×400 mL), filtrate combined and concentrated under reduced pressure. The crude residue, thus obtained, was purified by using MPLC (manually packed SiO2 cartridge, 100-200 mesh; 20% EtOAc in hexanes) to afford (±)-3 as an off-white solid.

Yield: 9.2 g (60%)

Note: Additional 4.39 g of (±)-3 was obtained from another batch

SFC: enantiomeric ratio=1:1 (tR=3.09 min and 3.90 min; Column: CHIRALPAK-AS-H; Eluents: CO2 and 0.2% formic acid in isopropanol and ACN mixture).

### Step 2:

The enantiomers of 10 g of (±)-3 were separated by SFC (Column: Chiralpak ASH-(250*30) mm, 5 μm, Eluents: CO2 and 0.2% formic acid in isopropyl alcohol and acetonitrile mixture) to obtain 3-Isomer-1 (tR=3.01 min) and 3-Isomer-2 (tR=3.7 min) as off-white solids.

Yield: 3-Isomer-1=4 g and 3-Isomer-2=3.9 g

LCMS: Calculated C₁₁H₁₃BrO₃ is 272.0. Observed: Desired molecular ion not observed

SFC: 3-Isomer-1: 100%; tR=3.01 min (Column: CHIRALPAK-AS-H; Eluents: 0.2% formic acid in isopropyl alcohol and acetonitrile mixture).

3-Isomer-2: 96.6% (ee=95%); tR=3.7 min (Column: CHIRALPAK-AS-H; Eluents: 0.2% formic acid in isopropyl alcohol and acetonitrile mixture).

Individually, both the isomers were taken for further conversion.

### Step 3:

To a solution of 3-isomer-1 (4 g, 14.65 mmol) in 1,4-dioxane (80 mL), were added bis(pinacolato)diboron (7.44 g, 29.3 mmol) and potassium acetate (3.59 g, 36.6 mmol) at room temperature and the resulting mixture purged with nitrogen for 5 min. To this reaction mixture, PdCl2(dppf)·DCM adduct (1.196 g, 1.465 mmol) was added and the reaction mixture stirred at 80° C. for 16 h. The reaction mixture was cooled to room temperature and filtered through a Celite pad. The pad was washed with EtOAc (90 mL×3); The combined filtrate was washed with brine solution (50 mL), dried over anhydrous sodium sulphate, filtered, and concentrated under reduced pressure. The crude residue, thus obtained, was purified by using MPLC (manually packed SiO2 cartridge, 100-200 mesh size; 15% EtOAc in hexanes) to afford 4-Isomer-1 as a colorless liquid.

Yield: 4.2 g (80%)

LC-MS: Calculated for C17H25BO5 is 320.18, Observed: 321.3 [M+1]⁺

### Step 4:

To a solution of 4-Isomer-1 (4.5 g, 14.05 mmol) in DCM (60 mL), were added 3,4-dihydro-2H-pyran (1.928 mL, 21.08 mmol) and PPTS (0.353 g, 1.405 mmol) at room temperature and stirred for 36 h. The reaction mixture was quenched with water (50 mL) and extracted with DCM (3×50 mL). The combined organic layer was washed with brine solution (30 mL), dried over sodium sulfate, filtered, and concentrated under reduced pressure. The crude residue thus obtained was purified by using MPLC (manually packed SiO2 cartridge: 230-400 mesh size; 5% MeOH in DCM) to afford 5-Isomer-1 as a colorless liquid.

Yield: 4.1 g (68%)

LC-MS: Calculated for C22H33BO6 is 404.31. Observed: 404.4 [M]⁺

### Step 5:

To a stirred solution of 5-Isomer-1 (4.34 g, 10.73 mmol) in acetonitrile (40 mL) and water (40 mL), were added 6 (3 g, 7.15 mmol) and K2CO3 (2.97 g, 21.46 mmol) at room temperature, and the reaction mixture purged with nitrogen for 5 min. To this reaction mixture, PdCl2(dtbpf) (0.466 g, 0.715 mmol) was added and the purging continued for another 2 min.

The reaction mixture was stirred at 80° C. for 16 h. The reaction mixture was quenched with water (100 mL) and extracted with EtOAc (3×80 mL). The combined organic layer was washed with brine solution (50 mL), dried over sodium sulfate, filtered, and concentrated under reduced pressure. The crude residue, thus obtained, was purified by using MPLC (manually packed SiO2 cartridge, 230-400 mesh size; 5% MeOH in DCM) to afford 7-Isomer-1 as a brown gum.

Yield: 2.3 g (51%)

LC-MS: Calculated for C36H44N2O7 is 616.75, Observed: 617.2 [M+1]⁺

### Step 6:

To a stirred solution of 7-Isomer-1 (300 mg, 0.486 mmol) in MeOH (20 mL), was added p-toluenesulfonic acid monohydrate (278 mg, 1.459 mmol) at 0° C., and the reaction mixture stirred at room temperature for 2 h. The reaction was quenched with saturated NaHCO3 solution (20 mL) and extracted with 10% MeOH in DCM (3×50 mL). The combined organic layer was washed with brine solution (30 mL), dried over sodium sulfate, filtered, and concentrated under reduced pressure. The crude residue, thus obtained, was purified by using MPLC (manually packed SiO2 cartridge: 230-400 mesh size; 5% MeOH in DCM) to afford Compound 113 as an off-white solid.

Yield: 100 mg (45%)

LC-MS: Calculated for C26H28N2O5 is 448.52, Observed: 449.3 [M+1]⁺

1H-NMR (400 MHz, DMSO-d₆): δ 7.66 (d, J=8.4 Hz, 2H), 7.53-7.5 (m, 4H), 7.36 (d, J=0.4 Hz, 1H), 7.11 (d, J=8.4 Hz, 1H), 6.84 (s, 1H), 5.69 (t, J=6.0 Hz, 1H), 5.54 (t, J=5.6 Hz, 1H, exchanges with D2O), 5.48 (d, J=4.0 Hz, 1H, exchanges with D2O), 5.38 (d, J=5.6 Hz, 1H, exchanges with D2O), 4.96-4.92 (m, 1H), 4.72 (d, J=4.0 Hz, 1H), 4.23 (br s, 1H), 4.09 (dd, J=4.0, 10.2 Hz, 1H), 3.93 (dd, J=4.0, 9.4 Hz, 1H), 3.87 (t, J=6.0 Hz, 2H), 3.79 (d, J=10.0 Hz, 1H), 3.63 (d, J=9.2 Hz, 1H), 2.19 (s, 3H), 1.51 (d, J=6.4 Hz, 3H).

SFC: 98.5%; tR=9.2 min (Column: LUX-I-Amylose3; Eluents: 0.5% isopropyl amine in acetonitrile and MeOH). trans-geometry at tail. Diastereomeric ratio=98.5:0.5 (based on SFC)

Compound 114 was synthesized in an identical fashion starting with 3-Isomer-2 by using the steps detailed for Compound 113.

LC-MS: Calculated for C26H28N2O5 is 448.52, Observed: 449.0 [M+1]⁺

1H-NMR (400 MHz, DMSO-d₆): δ 7.66 (d, J=8.4 Hz, 2H), 7.53-7.50 (m, 4H), 7.38 (s, 1H), 7.12 (d, J=8.4 Hz, 1H), 6.87 (s, 1H), 5.70 (t, J=6.0 Hz, 1H), 5.54 (t, J=5.6 Hz, 1H, exchanges with D2O), 5.48 (d, J=3.6 Hz, 1H, exchanges with D2O), 5.40 (d, J=5.2 Hz, 1H, exchanges with D2O), 4.98-4.92 (m, 1H), 4.72 (d, J=4.0 Hz, 1H), 4.23 (br s, 1H), 4.09 (dd, J=4.0, 10.0 Hz, 1H), 3.93 (dd, J=4.0, 9.2 Hz, 1H), 3.89-3.80 (m, 2H), 3.79 (d, J=10.0 Hz, 1H), 3.63 (dd, J=1.2, 9.4 Hz, 1H), 2.19 (s, 3H), 1.51 (d, J=6.4 Hz, 3H).

SFC: 95.3%; tR=8.44 min (Column: LUX-I-Amylose3; Eluents: 0.5% isopropyl amine in acetonitrile and MeOH). trans-geometry at tail. Diastereomeric ratio=95.3:3.3.

### Example A64: Synthesis of Compounds 115 and 116

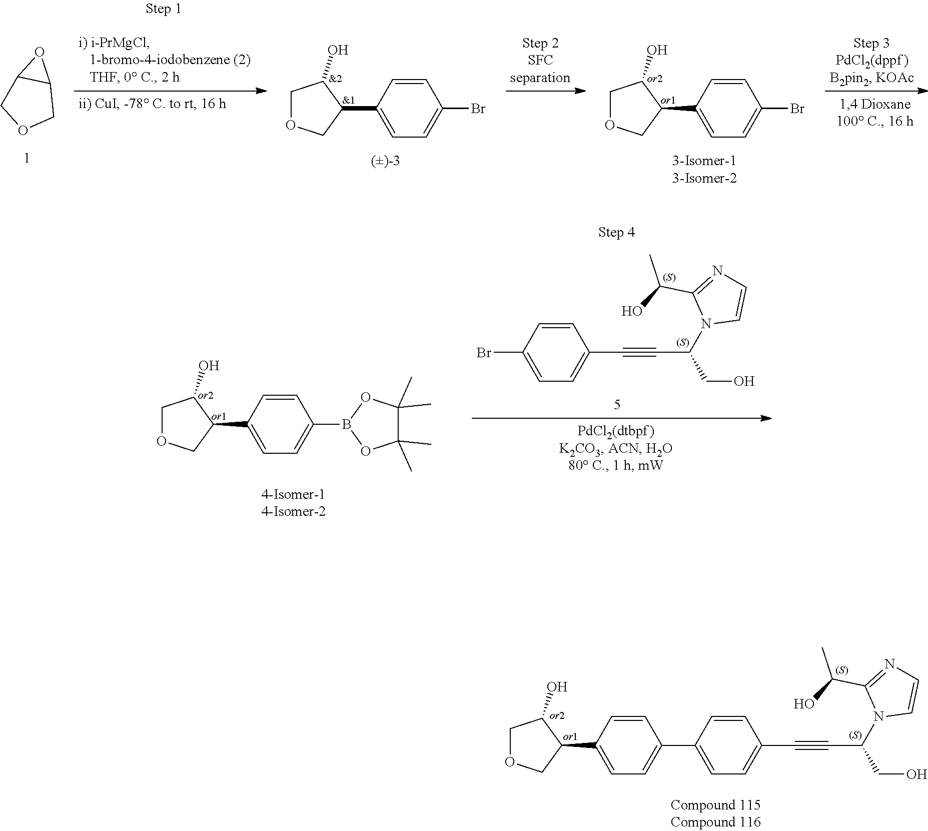

### Step 1:

To a stirred solution of 1-bromo-4-iodobenzene (2, 32.9 g, 116 mmol) in THF (340 mL), was added isopropylmagnesium chloride (58.1 mL, 116 mmol) drop wise at 0° C., and stirred for 2 h at 0° C. To a stirred solution of 3,4-epoxytetrahydrofuran (1, 5 g, 58.1 mmol) in THF (60 mL), was added copper(I) iodide (1.106 g, 5.81 mmol) at 0° C. To this reaction mixture, the above solution was added drop wise at −78° C. The reaction mixture was allowed to attain room temperature and stirred for 16 h. The reaction mixture was quenched with sat. ammonium chloride solution (300 mL) and extracted with EtOAc (250 mL×2). The combined organic layer was washed with brine (250 mL), dried over anhydrous Na₂SO₄, filtered and concentrated under reduced pressure. The resulting crude residue was purified by MPLC (using manually packed cartridge, SiO₂ 230-400 mesh; 20% EtOAc in hexanes) to obtain racemic (±)-3 as a white solid.

Yield: 10 g (70%)

### Step 2:

10 g of (±)-3 was separated by using SFC (Column: LUX I_A3 (250×30) mm, 5 μm; Eluents: CO2:0.5% isopropyl amine in MeOH [60:40]) to get 3-Isomer-1 (t_{R}=3.0 min) and 3-Isomer-2 (t_{R}=4.7 min) as an off-white solids.

Yield: 3-Isomer-1=4 g and 3-Isomer-2=3.4 g

SFC purity=3-Isomer-1: 97.5% (ee=95%) and 3-Isomer-2: 98.5% (ee=97%)

Both the isomers (3-Isomer-1 and 3-Isomer-2) were taken independently for further conversion.

The synthesis of Compound 115 was carried out using 3-Isomer-1.

### Step 3:

To a stirred solution of 3-Isomer-1 (1 g, 4.11 mmol) in 1,4-dioxane (20 mL), were added bis(pinacolato)diboron (1.567 g, 6.17 mmol) and potassium acetate (1.211 g, 12.34 mmol) at room temperature. The reaction mixture was purged with nitrogen for 5 min. To this reaction mixture, PdCl2(dppf) (0.301 g, 0.411 mmol) was added. The mixture was stirred at 100° C. for 16 h. The reaction was quenched with water (100 mL) and extracted with EtOAc (50 mL×2). The combined organic layer was washed with brine (50 mL), dried over anhydrous Na₂SO₄, filtered and concentrated under reduced pressure to get the crude product. The crude residue was purified by MPLC (manually packed cartridge, SiO₂ 230-400 mesh; 30% EtOAc in hexanes) to obtain 4-Isomer-1 as a pale-yellow gum.

Yield: 1.1 g (90%)

LC-MS: Calculated for C16H23BO4 is 290.1, Observed: 289.3 [M−1]⁻

### Step 4:

To a stirred solution of 5 (200 mg, 0.597 mmol) in acetonitrile (2 mL) and water (2 mL), were added 4-Isomer-1 (260 mg, 0.895 mmol) and K2CO3 (247 mg, 1.790 mmol) at room temperature. The resulting reaction mixture was purged with nitrogen for 5 min. To this reaction mixture, PdCl2(dtbpf) (38.9 mg, 0.060 mmol) was added and the purging continued for another 2 min. The resulting reaction was irradiated at 80° C. for 1 h in a microwave reactor. The reaction mixture was quenched with water (30 mL), and then extracted with 10% MeOH in DCM (10 mL×2). The combined organic extract was dried over anhydrous Na₂SO₄, filtered and concentrated under reduced pressure. The crude residue was purified by preparative HPLC (Column: X-BRIDGE C18 (19×250 mm) 5 μm; Eluents: 10 mM ammonium bicarbonate in water and acetonitrile) to obtain Compound 115 as a white solid.

Yield: 45 mg (18%)

LC-MS: Calculated for C25H26N2O4 is 418.4, Observed: 419.3 [M+1]⁺

1H-NMR (400 MHz, DMSO-d6): δ 7.69 (d, J=8.40 Hz, 2H), 7.65 (d, J=8.40 Hz, 2H), 7.54 (d, J=8.40 Hz, 2H), 7.38-7.36 (m, 2H), 7.36 (app d, J=0.80 Hz, 1H), 6.84 (app d, J=0.80 Hz, 1H), 5.68 (t, J=6.00 Hz, 1H), 5.54 (t, J=5.60 Hz, 1H, exchanges with D2O), 5.37 (d, J=5.60 Hz, 1H, exchanges with D2O), 5.30 (d, J=4.40 Hz, 1H, exchanges with D2O), 4.96-4.93 (m, 1H), 4.26 (t, J=4.80 Hz, 1H), 4.18 (dd, J=7.20, 8.40 Hz, 1H), 3.98 (dd, J=5.60, 9.20 Hz, 1H), 3.87 (t, J=6.00 Hz, 2H), 3.75 (dd, J=6.40, 8.60 Hz, 1H), 3.58 (dd, J=4.00, 9.00 Hz, 1H), 3.26-3.23 (m, 1H), 1.51 (t, J=6.80 Hz, 3H). Single isomer; unknown stereochemistry at tail. dr=100:0 based on SFC (Whelk-(R,R)_0.5% IPAm in MeOH t_{R}=5.88 min). Elution order has changed compared to the intermediate-3 of the synthesis.

Compound 116 was synthesized by using the same procedure of Step-3 and Step-4 of Compound 115.

LC-MS: Calculated for C25H26N2O4 is 418.4, Observed: 419.3 [M+1]⁺

1H-NMR (400 MHz, DMSO-d6): δ 7.69 (d, J=8.40 Hz, 2H), 7.65 (d, J=8.00 Hz, 2H), 7.54 (d, J=8.00 Hz, 2H), 7.38-7.36 (m, 3H), 6.84 (s, 1H), 5.68 (t, J=6.00 Hz, 1H), 5.54 (t, J=5.60 Hz, 1H, exchanges with D2O), 5.37 (d, J=5.60 Hz, 1H, exchanges with D2O), 5.30 (d, J=4.80 Hz, 1H, exchanges with D2O), 4.96-4.93 (m, 1H), 4.26 (t, J=4.40 Hz, 1H), 4.18 (t, J=8.00 Hz, 1H), 3.97 (dd, J=5.60, Hz, 1H), 3.87 (t, J=5.60 Hz, 2H), 3.75 (dd, J=6.00, 8.40 Hz, 1H), 3.58 (dd, J=4.00, 9.00 Hz, 1H), 3.26-3.22 (m, 1H), 1.51 (t, J=6.40 Hz, 3H). Single isomer; unknown stereochemistry at tail. dr=100:0 based on SFC (Whelk-(R,R)_0.5% IPAm in MeOH t_{R}=5.36 min). Elution order has changed compared to the intermediate-3 of the synthesis.

### Example A65: Synthesis of Compound 117

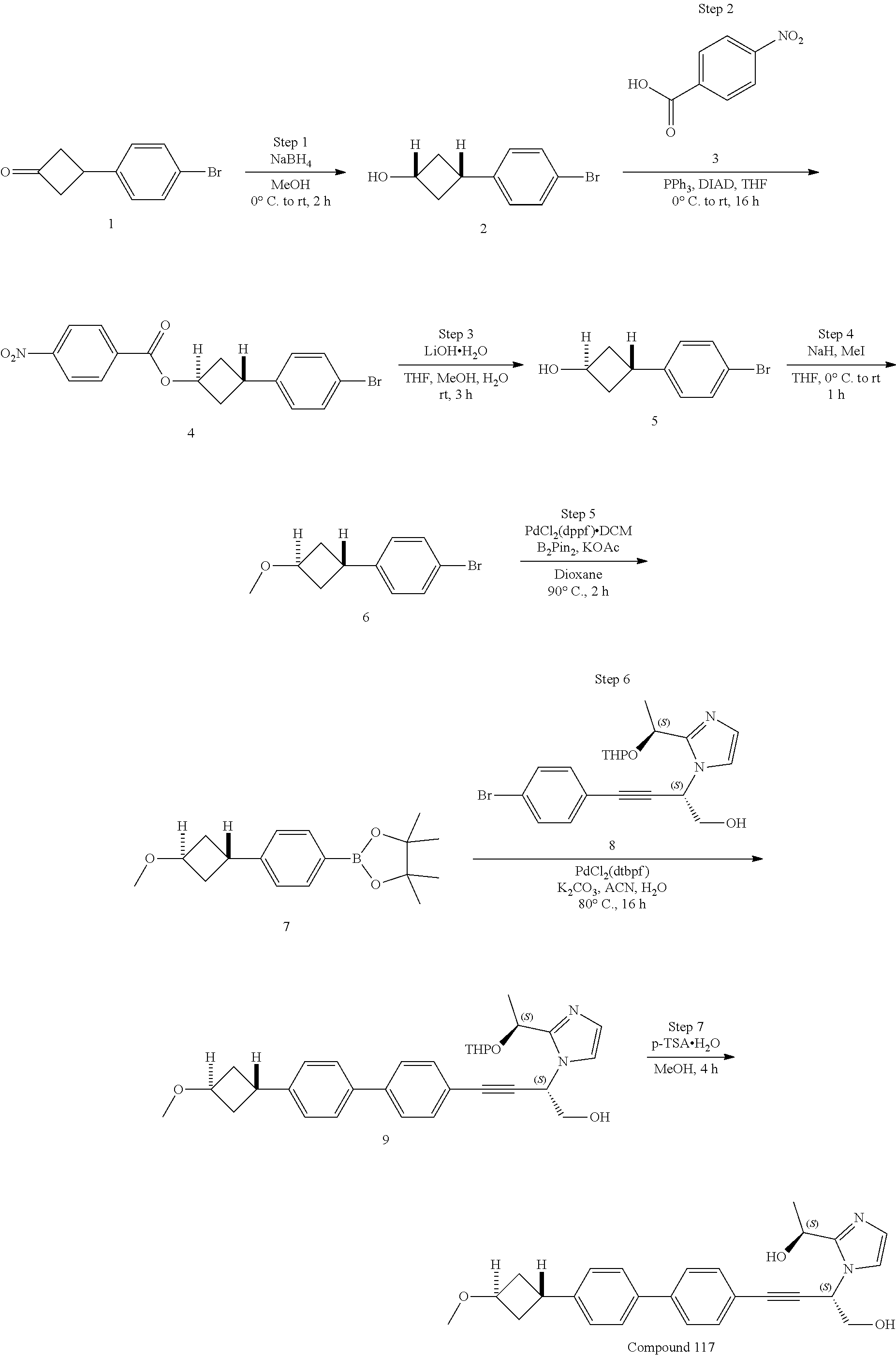

### Step 1:

To a cooled solution of 3-(4-bromophenyl)cyclobutan-1-one (1, 8 g, 35.5 mmol) in MeOH (100 mL), was added NaBH4 (0.672 g, 17.77 mmol) in portions at 0° C. After the complete addition, the resulting reaction mixture was allowed to room temperature and stirred for 2 h. The reaction was cooled to 0° C. and quenched with sat. NH₄Cl solution (20 mL). The reaction mixture was evaporated to remove methanol and extracted with EtOAc (250 mL×2). The combined organic extract was washed with brine (20 mL), dried over anhydrous sodium sulfate, filtered, and concentrated to afford crude as a pale-yellow liquid, which was purified by MPLC (manually packed cartridge; SiO2 230-400 mesh size; 20% EtOAc in hexanes) to get 2 as a yellow solid.

Yield: 6 g (72%)

### Step 2:

To a cooled solution of 2 (6 g, 25.6 mmol) in THF (100 mL), were added 4-nitrobenzoic acid (3, 4.71 g, 28.2 mmol), triphenylphosphine (8.07 g, 30.8 mmol) and DIAD (5.41 mL, 30.8 mmol) at 0° C. The resulting reaction mixture was allowed to warm to room temperature and stirred for 16 h. The reaction mixture was quenched with water (40 mL) and extracted with EtOAc (100 mL×2). The combined organic extract was washed with brine (30 mL), dried over anhydrous sodium sulfate, filtered, and concentrated under reduced pressure to afford a crude residue, which was purified by MPLC (manually packed cartridge; SiO2 230-400 mesh size; 10% EtOAc in hexanes) to get 4 as a white solid.

Yield: 6.5 g (60%)

LC-MS: Calculated for C17H14BrNO4 is 376.21, Observed: 376.8 [M]⁻ and 374.8 [M−2]⁻

### Step 3:

To a solution of 4 (6.5 g, 15.55 mmol) in a mixture of THF (10 mL), water (2.5 mL) and MeOH (10 mL), was added lithium hydroxide monohydrate (1.958 g, 46.6 mmol) at room temperature. The resulting reaction mixture was stirred at room temperature for 3 h. The reaction mixture was evaporated to remove volatiles. To the obtained residue, water (15 mL) was added and extracted with EtOAc (50 mL×5). The combined organic extract was washed with brine (15 mL), dried over anhydrous sodium sulfate, filtered, and concentrated under reduced pressure to afford crude residue, which was purified by MPLC (manually packed cartridge; SiO2 230-400 mesh size; 30% EtOAc in hexanes) to get 5 as a white solid.

Yield: 3.0 g (68%)

### Step 4:

To the stirred solution of sodium hydride (1.014 g, 21.14 mmol) in THE (30 mL), was added 5 (3 g, 10.57 mmol) at 0° C. and stirred for 10 min. Then, iodomethane (1.32 mL, 21.14 mmol) was added dropwise at 0° C. After the complete addition, the resulting reaction mixture was warmed to room temperature and stirred for 1 h. The reaction mixture was quenched with ice-cold water and sat. NH₄Cl solution (15 mL). This was extracted with EtOAc (30 mL×3). The combined organic extract was washed with brine (15 mL), dried over anhydrous sodium sulphate, filtered, and concentrated under reduced pressure to afford a crude residue, which was purified by MPLC (manually packed cartridge; SiO2 230-400 mesh size; 20% EtOAc in hexanes) to get 6 as white solid.

Yield: 2.5 g (78%).

The stereochemistry was confirmed by nOe studies. The nOe studies showed no interactions between H4 (3.57 ppm) and H2 (4.07 ppm).

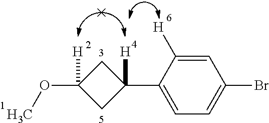

### Step 5:

To the stirred solution of 6 (2.5 g, 10.37 mmol) in 1,4-dioxane (15 mL), were added bis(pinacolato)diboron (3.42 g, 13.48 mmol) and KOAc (2.035 g, 20.74 mmol) at 0° C. The reaction mixture was purged with nitrogen for 5 min. To this reaction mixture, PdCl₂(dppf) (0.759 g, 1.037 mmol) was added. The resulting reaction mixture was heated to 90° C. for 2 h. The reaction mixture was diluted with water (8 mL) and extracted with EtOAc (2×20 mL). The combined organic extract was washed with brine (8 mL), dried over anhydrous sodium sulphate, filtered, and concentrated under reduced pressure to afford crude residue, which was purified by MPLC (manually packed cartridge; SiO2 230-400 mesh size; 10% EtOAc in hexanes) to get 7 as a white solid.

Yield: 2.2 g (73%)

### Step 6:

To the stirred solution of 7 (0.973 g, 3.04 mmol) in MeCN (4 mL) and water (4 mL), were added K₂CO₃ (0.969 g, 7.01 mmol) and 8 (1.0 g, 2.337 mmol) at room temperature. The reaction mixture was purged with nitrogen for 2 min. To this reaction mixture, PdCl2(dtbpf) (0.152 g, 0.234 mmol) was added. The resulting reaction mixture was heated to 80° C. and stirred for 16 h. The reaction mixture was diluted with ice-cold water (8 mL) and extracted with 5% MeOH in DCM (16 mL×3). The combined organic extract was washed with brine (8 mL), dried over anhydrous sodium sulphate, filtered, and concentrated under reduced pressure to afford crude residue, which was purified by MPLC (manually packed cartridge; SiO2 230-400 mesh size; 5% MeOH in DCM) to get 9 as brown sticky solid.

Yield: 405 mg (32%)

LC MS: Calculated for C31H36N2O4 is 500.27, Observed: 501.2 [M+1]⁺

### Step 7:

To the stirred solution of 9 (135 mg, 0.243 mmol) in MeOH (30 mL), was added toluene-4-sulfonic acid monohydrate (138 mg, 0.728 mmol) at 0° C. The resulting reaction mixture was stirred at room temperature for 4 h. Two more batches were performed on 135 mg of 9. All the batches were quenched with sat. NaHCO3 solution at 0° C. Then, all the reaction mixtures combined for work up and purification. The combined aqueous layer was extracted with DCM (100 mL×3). The combined organic extract was washed with sat. NaHCO₃ solution (3×5 mL), brine (8 mL), dried over anhydrous sodium sulphate, filtered, and concentrated to afford a crude residue, which was purified by reverse phase preparative HPLC purification to obtain Compound 117 as white solid.

Yield: 145 mg (Combined yield for three batches). LC MS: Calculated for C26H28N2O3 is 416.21, Observed: 417.0 [M+1]⁺

SFC purity: 95.6%. The final compound has trans- geometry in tail part and is a mixture of diastereomers.

### Example A66: Synthesis of Compound 118

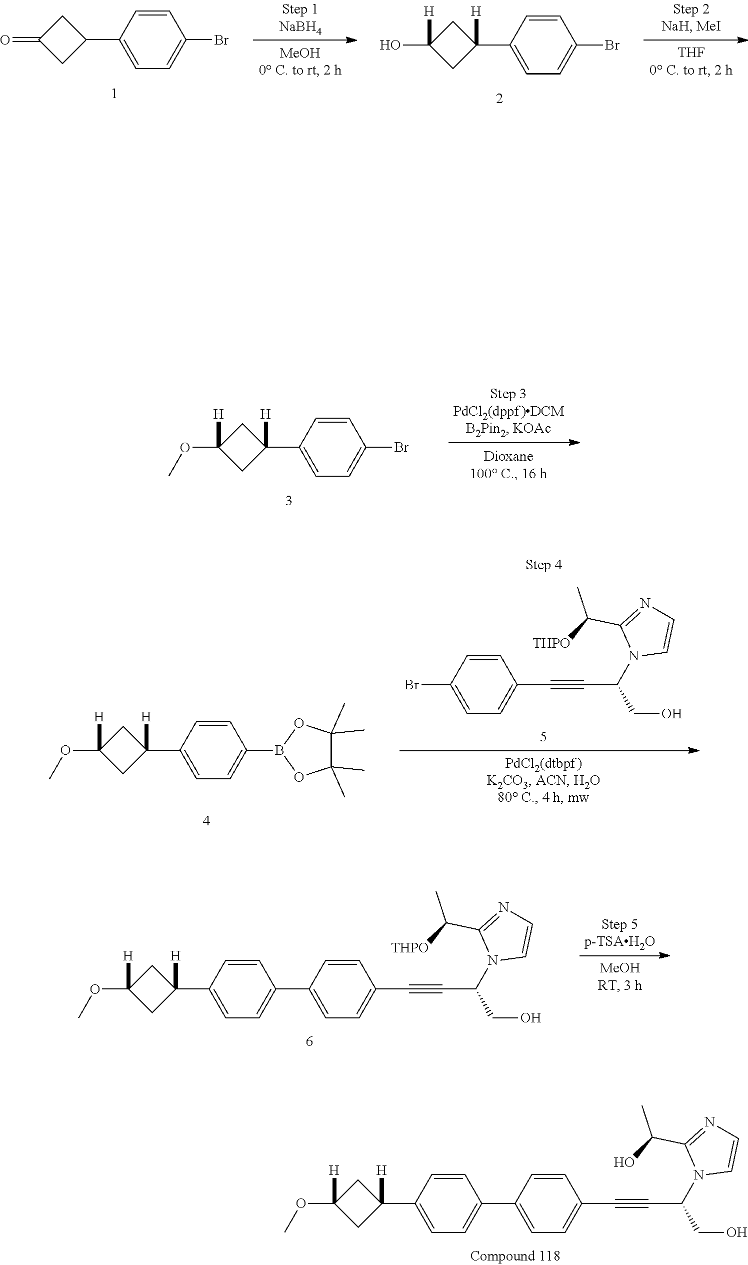

### Step-1:

To a stirred solution of 3-(4-bromophenyl) cyclobutan-1-one (1, 3.2 g, 14.22 mmol) in MeOH (30 mL), was added sodium borohydride (0.27 g, 7.11 mmol) portion wise at 0° C. The resulting reaction mixture was stirred at room temperature for 2 h. The volatiles were removed under reduced pressure. The crude residue was quenched with water (50 mL) and extracted with EtOAc (50 mL×2). The combined organic layer was dried over anhydrous Na₂SO₄, filtered and concentrated reduced pressure to afford 2 as pale-yellow solid. The crude product was taken to the next step without any purification.

Yield=2.0 g (59%)

Reduction of 3-substituted cyclobutanones is known to yield cis- alcohols irrespective of the nature of reducing agent vide J. Org. Chem. 2020, 85, 7803 and references cited therein.

### Step-2:

To a stirred solution of 2 (1.5 g, 6.60 mmol) in THF (20 mL), was added NaH (58% in paraffin oil; 0.5 g, 13.21 mmol) at 0° C. in portion wise manner. The reaction mixture was stirred at same temperature for 30 min. To this reaction mixture, iodomethane (0.411 mL, 6.60 mmol) was added drop wise. The reaction mixture was stirred at room temperature for 2 h. The reaction mixture was cooled to 0° C., quenched with ice cold water (50 mL) and extracted with EtOAc (2×50 mL). The combined organic extract was dried over Na₂SO₄, filtered and concentrated under reduced pressure. The resulting crude mass was purified by MPLC (manually packed cartridge, SiO2, 230-400 mesh size; 6% EtOAc in hexanes) to afford 3 as pale yellow liquid.

Yield=1.0 g (57%)

The predominant conformation of 3 was confirmed on the basis of 1D NOE data.

### Step-3:

To a solution of 3 (1.0 g, 4.15 mmol) in dioxane (20 mL), were added potassium acetate (1.22 g, 12.44 mmol) and bis(pinacolato)diboron (1.580 g, 6.22 mmol) at room temperature. The resulting mixture was purged with nitrogen for 10 min. To this reaction mixture, PdCl2(dppf)·DCM adduct (0.34 g, 0.415 mmol) was added and the purging continued for 2 min. The reaction mixture was heated at 100° C. for 16 h. The inorganic solids were filtered through Celite pad and washed with EtOAc (50 mL). The filtrate was concentrated under reduced pressure afford a crude residue. The crude residue was purified by MPLC (manually packed SiO2 cartridge, 100-200 mesh size; 5% EtOAc in hexanes) to afford 4 as yellow coloured sticky compound.

Yield: 900 mg (68%)

### Step-4:

To a stirred solution of 5 (850 mg, 2.027 mmol) in ACN (10 mL) and water (10 mL), were added boronate ester 4 (876 mg, 3.04 mmol) and K2CO3 (840 mg, 6.08 mmol) at room temperature. The reaction mixture was degassed with nitrogen for 5 min. To this reaction mixture, PdCl2(dtbpf) (0.132 g, 0.203 mmol) was added and the degassing continued for another 2 min. The resulting reaction mixture was stirred at 80° C. for 16 h. The reaction mixture was quenched with water (50 mL), extracted with 10% MeOH in DCM (50 mL×2). The combined organic layer was washed with brine solution (30 mL), dried over anhydrous Na2SO4, filtered and concentrated under reduced pressure. The resulting crude product was purified by MPLC (manually packed SiO2 cartridge, 230-400 mesh size; 5% MeOH in DCM) to afford 6 as pale-brown solid.

Yield: 700 mg (66%)

LC-MS: Calculated for C31H36N2O4 is 500.64, Observed: 501.3 [M+1]⁺

### Step-5:

To a stirred solution of (6, 0.7 g, 1.398 mmol) in MeOH (10 mL), was added p-toluenesulfonic acid monohydrate (0.8 g, 4.19 mmol) at 0° C. The reaction mixture was stirred at room temperature for 3 h. The volatiles were evaporated under reduced pressure, the resulting residue was basified with 10% NaHCO3 solution (20 mL) and extracted with 10% MeOH in DCM (50 mL×2). The combined organic extract was washed with brine (30 mL), dried over anhydrous Na₂SO₄, filtered and concentrated under reduced pressure. The crude mass was purified by reversed phase preparative HPLC (Column: Zorbax C18 (150*21.2 mm) 5 μm; Eluents: 10 mM ammonium bicarbonate in water and ACN) to afford Compound 118 as white solid.

Yield=16 mg (27%)

LC-MS: Calculated for C26H28N2O3 is 416.521, Observed: 417.2 [M+1]⁺

### Example A67: Synthesis of Compound 119

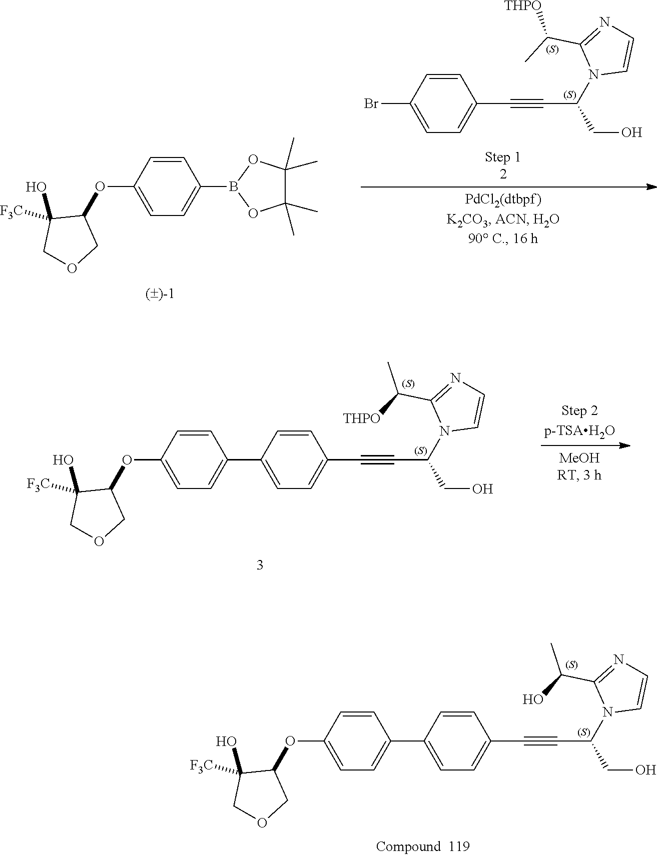

### Step-1:

To a solution of (1)-1 (1 g, 2.67 mmol) in ACN (8 mL) and water (8 mL), were added 2 (1.075 g, 3.21 mmol) and potassium carbonate (1.108 g, 8.02 mmol) at room temperature. The resulting mixture was purged with nitrogen for 5 min. To this reaction mixture, PdCl2(dtdf) (0.174 g, 0.267 mmol) was added and the resulting mixture stirred at 90° C. for 16 h. The reaction mixture was diluted with water (60 mL) and extracted with 10% MeOH in DCM (3×50 mL). The combined organic layer was washed with brine (50 mL), dried over sodium sulfate, filtered and the filtrate concentrated under reduced pressure. The resulting crude mass was purified by MPLC (manually packed SiO2 cartridge, 230-400 mesh size; 7% MeOH in DCM) to afford 3 as a pale-brown solid.

Yield: 730 mg (45%)

LC-MS: Calculated for C31H33F3N2O6 is 586.23, Observed: 587.2 [M+H]⁺

### Step-2:

To a stirred solution of 3 (0.7 g, 1.193 mmol) in MeOH (10 mL) was added p-toluenesulfonic acid monohydrate (0.681 g, 3.58 mmol) at 0° C. and the reaction mixture stirred at room temperature for 3 h. The reaction mixture was quenched with 10% aq. sodium bicarbonate solution (30 mL), extracted with 10% MeOH in DCM (3×50 mL). The combined organic layer was washed with brine (50 mL), dried over sodium sulfate, filtered and the filtrate concentrated under reduced pressure. The resulting compound was purified by reverse phase preparative HPLC (Column: X-SELECT C18 (250*19 mm) 5 μm, Eluents: 0.1% ammonium bicarbonate in water and acetonitrile) to get Compound 119 as an off-white solid.

Yield: 110 mg (18%)

LC-MS: Calculated for C26H25F3N2O5 is 502.17, Observed: 503.0 [M+H]⁺. The final compound is a mixture of diastereomers, racemic at tail.

### Example A68: Synthesis of Compound 120

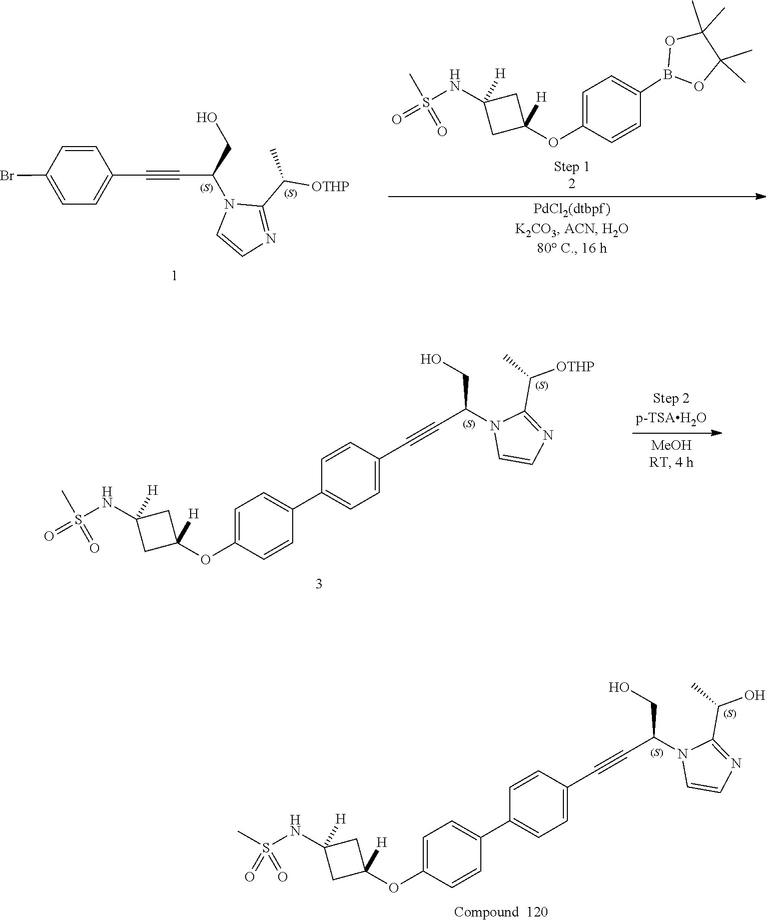

### Step-1:

To a stirred solution of 1 (1.5 g, 3.58 mmol) in acetonitrile (50 mL) and water (10 mL), were added boronate 2 (1.577 g, 4.29 mmol) and K2CO3 (0.989 g, 7.15 mmol). The reaction mixture was purged with nitrogen for 10 min. To this reaction mixture, PdCl2(dtbpf) (0.233 g, 0.358 mmol) was added and the purging continued for another 2 min. The reaction mixture was stirred at 80° C. for 16 h. The reaction was cooled to ambient temperature, following which water (30 mL) was added and extracted with 10% MeOH in DCM (3×50 mL). The combined organic extract was dried over anhydrous Na₂SO₄, filtered, and concentrated under reduced pressure. The resulting crude mass was purified by MPLC (manually packed cartridge; SiO₂, 230-400 mesh size; 15% MeOH in DCM) to afford 3 as a brown solid.

Yield: 1.0 g (43%)

LC-MS: Calculated for C₃₁H₃₇N₃O₆S is 579.71, Observed: 580.3 [M+1]⁺

### Step-2:

To a stirred solution of 3 (0.350 g, 0.604 mmol) in MeOH (45 mL), was added p-toluenesulfonic acid monohydrate (0.350 g, 1.840 mmol) at 0° C. The reaction mixture was stirred at room temperature for 4 h. The volatiles were removed under reduced pressure, and the resulting residue was basified with 10% NaHCO3 solution (10 mL) and extracted with DCM (50 mL×2). The combined organic extract was dried over anhydrous Na₂SO₄, filtered, and concentrated under reduced pressure. The crude mass was purified by reverse phase column purification (Column: C18-reversed phase SiO₂: Eluents: 10 mM ammonium bicarbonate in water and ACN) to afford the Compound 120 as a white solid. Yield: 60 mg (20%)

LC-MS: Calculated for C₂₆H₂₉N₃O₅S is 495.59, Observed: 496.0 [M+1]⁺. The final compound has trans-geometry in the tail part.

### Example A69: Synthesis of Compound 121

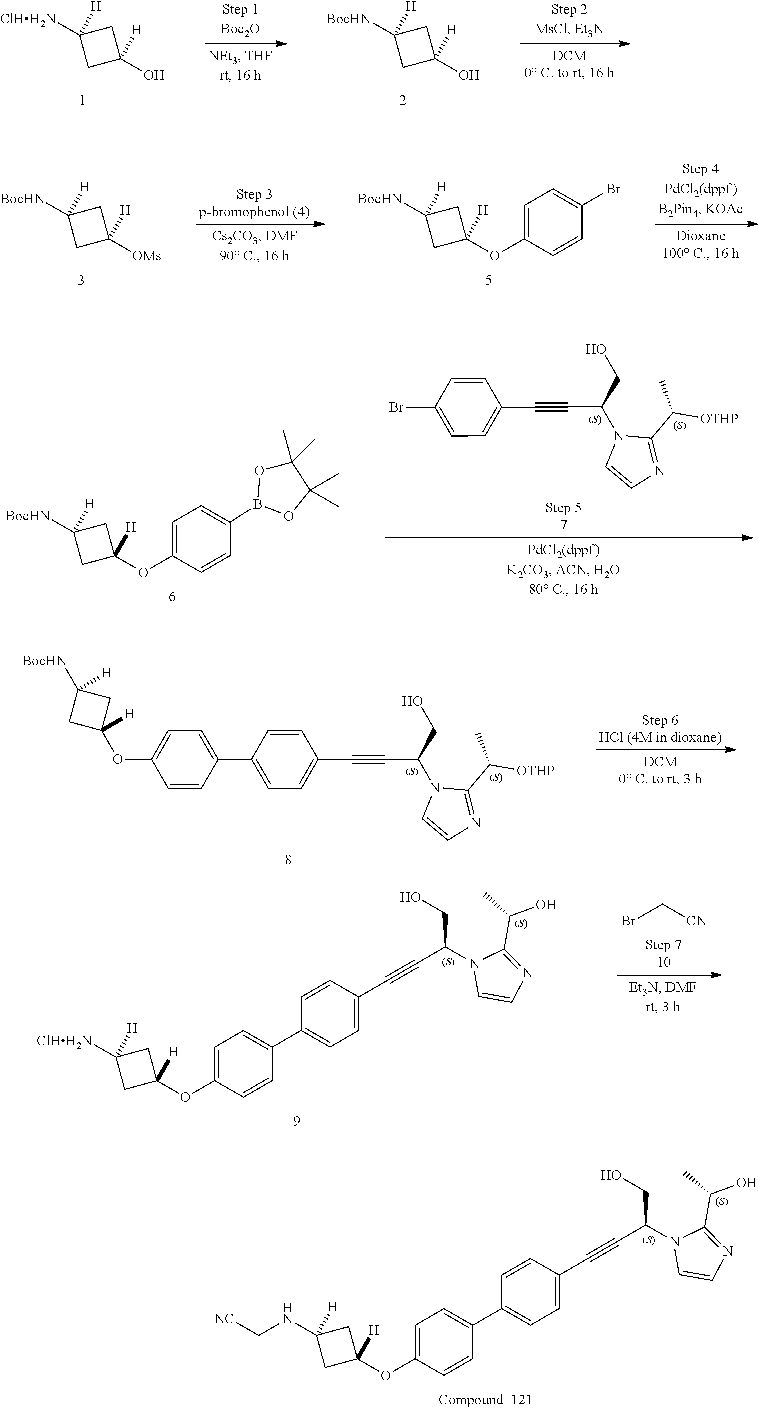

### Step-1:

To a stirred solution of rel-(1s,3 s)-3-aminocyclobutan-1-ol hydrochloride (1, 10 g, 81 mmol) in THF (500 mL), were added triethylamine (33.9 mL, 243 mmol) and Boc-anhydride (37.2 mL, 162 mmol) in dropwise manner. The reaction mixture was stirred at room temperature for 16 h. The volatiles were removed under reduced pressure. To the resulting residue water (100 mL) was added and extracted with EtOAc (100 mL×2). The combined organic extract was dried over anhydrous Na₂SO₄, filtered and concentrated under reduced pressure to afford 2 as a white solid, which was used in the next step without any further purification. Yield: 12 g (71%)

### Step-2:

To a stirred solution of 2 (6 g, 32.0 mmol) in DCM (50 mL), were added triethylamine (10.39 mL, 80 mmol) and methanesulphonyl chloride (3.47 mL, 44.9 mmol) at 0° C. The reaction mixture was stirred at room temperature for 16 h. The reaction was quenched with water (200 mL) and extracted with DCM (250 mL×2). The combined organic extract was washed with brine solution (150 mL), dried over anhydrous Na₂SO₄, filtered and concentrated under reduced pressure to afford 3 as a white solid.

Yield: 7 g (78%)

### Step-3:

To a solution of 4-bromophenol (4, 4.0 g, 23.12 mmol) in DMF (60 mL), were added caesium carbonate (11.30 g, 34.7 mmol) and 3 (7.36 g, 27.7 mmol) at room temperature. The reaction mixture was stirred at 90° C. for 16 h. The reaction was quenched with water (150 mL) and extracted with EtOAc (250 mL). The combined organic extract was washed with brine (30 mL), dried over anhydrous Na₂SO₄, filtered and concentrated under reduced pressure. The resulting crude mass was purified by MPLC (manually packed cartridge; SiO2 100-200 mesh size; 10% EtOAc in hexanes) to afford 5 as white solid. The stereochemistry of 5 was confirmed by nOe studies.

Yield: 3.0 g (87%)

### Step-4:

To a stirred solution of 5 (3 g, 8.77 mmol) in 1,4-dioxane (90 mL), were added potassium acetate (2.58 g, 26.3 mmol) and bis(pinacolato)diboron (3.34 g, 13.15 mmol) at room temperature. The resulting mixture was purged with nitrogen for 5 min. To this reaction mixture, was added PdCl2(dppf) (0.641 g, 0.877 mmol) and the purging continued for 2 min. The reaction mixture was stirred at 100° C. for 16 h. The inorganic solids were filtered through a Celite pad and washed with EtOAc (300 mL). The filtrate was concentrated under reduced pressure afford a crude residue. The resulting crude residue was purified by MPLC (manually packed cartridge; SiO2 230-400 mesh; 50% EtOAc in hexanes) to afford boronate 6 as a brown liquid.

Yield: 2.8 g (61%)

LC-MS: Calculated for C₂₁H₃₂BNO₅ is 389.29, Observed: 290.2 [M-Boc+1]⁺

### Step-5:

To a solution of 7 (1.8 g, 4.29 mmol) in 1,4-dioxane (50 mL) and water (8 mL), were added boronate 6 (2.507 g, 6.44 mmol) and K2CO3 (1.780 g, 12.88 mmol). The reaction mixture was purged with nitrogen for 5 min. To this reaction mixture, PdCl2(dppf) (0.314 g, 0.429 mmol) was added and the purging continued for another 2 min. The reaction mixture was stirred at 80° C. for 16 h. The inorganic solids were filtered through a Celite pad and washed with EtOAc (300 mL). The filtrate was concentrated under reduced pressure to afford a crude residue. The resulting crude residue was purified by MPLC (manually packed cartridge; SiO2 230-400 mesh size; 50% MeOH in DCM) to afford 8 as brown liquid.

Yield: 1 g (30%)

LC-MS: Calculated for C₃₅H₄₃N₃O₆ is 601.74, Observed: 603.2 [M+1]⁺

### Step-6:

To a solution of 8 (0.8 g, 1.329 mmol) in DCM (45 mL), was added HCl (4 M in dioxane, 22.5 mL, 90 mmol) at 0° C. The reaction mixture was stirred at room temperature for 3 h. The volatiles were evaporated under reduced pressure to get the crude residue. To the resulting residue n-hexanes (15 mL) was added at room temperature and stirred for 5 min. The precipitate obtained was filtered to afford 9 as a brown solid.

Yield: 600 mg (90%)

LC-MS: Calculated for C₂₅H₂₇N₃O₃ is 417.51 (for parent compound), Observed: 418.3 [M+1]⁺

### Step-7:

To a stirred solution of 9 (600 mg, 1.322 mmol) in DMF (10 mL), were added triethylamine (0.4 mL, 2.85 mmol) and 2-bromoacetonitrile (10, 0.1 mL, 1.436 mmol) at room temperature. The resulting reaction mixture was stirred at room temperature for 3 h. The volatiles were evaporated under reduced pressure to get the crude residue, which was purified by reversed phase column purification (Eluents: 10 mM ammonium bicarbonate in water and ACN) to afford the product as an off-white solid.

Yield: 70 mg (18%)

LC-MS: Calculated for C₂₇H₂₈N₄O₃ is 456.55, Observed: 457.0 [M+1]⁺

### Example A70: Synthesis of Compound 122

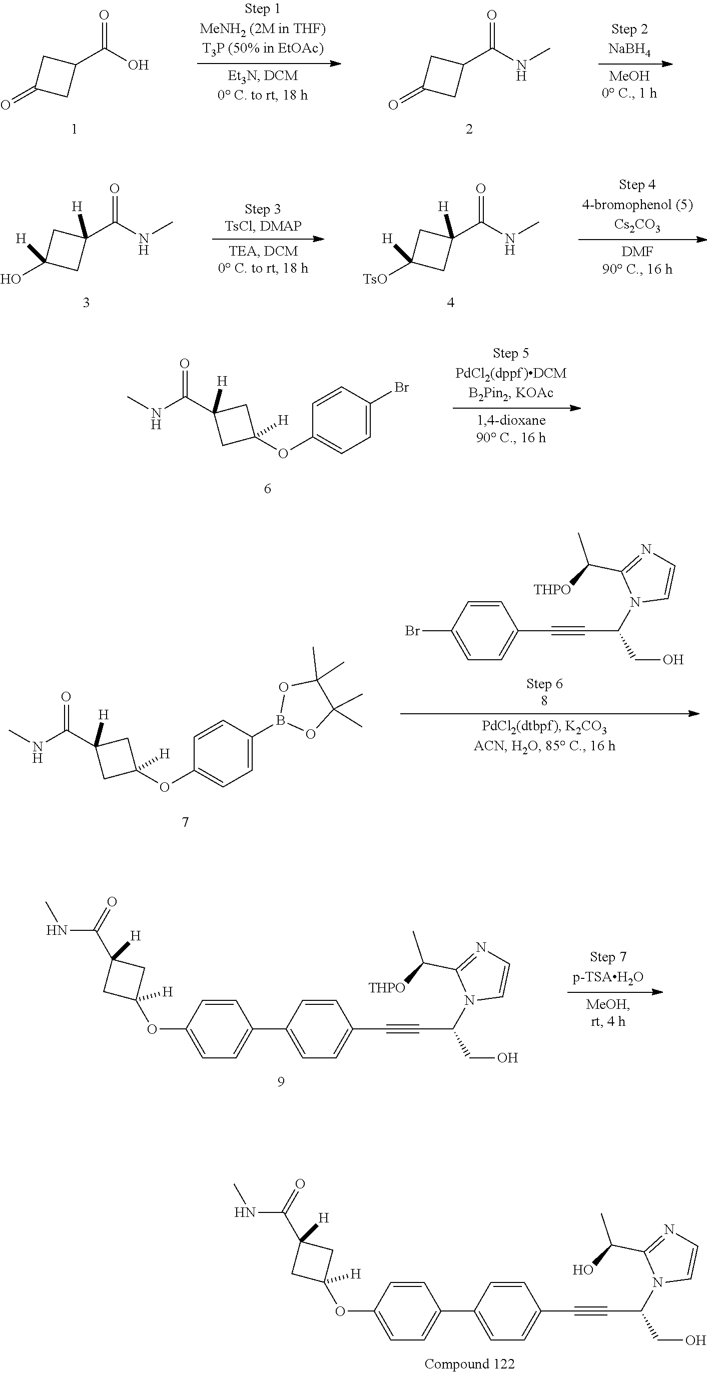

### Step-1:

To the stirred solution of 3-oxocyclobutane-1-carboxylic acid (1, 10 g, 88 mmol) in DCM (100 mL), were added methylamine (2 M in THF, 65.7 mL, 131 mmol) and T₃P (50% in EtOAc) (80 mL, 131 mmol) at 0° C. and the resulting reaction mixture allowed to attain room temperature and stirred for 18 h. The reaction mixture was quenched with water (20 mL), basified using 10% NaOH solution (80 mL) and extracted with 10% MeOH in DCM (6×100 mL). The combined organic extract was dried over anhydrous Na2SO4, filtered and concentrated under reduced pressure to get 2 as an off-white solid. The crude product was used in the next step without any further purification.

Yield: 9.5 g (73%) (crude product)

### Step-2:

To a stirred solution of 2 (9.0 g, 60.2 mmol) in MeOH (90 mL), was added NaBH₄ (1.138 g, 30.1 mmol) at 0° C. in portions, and the resulting reaction mixture stirred at 0° C. for 1 h. The reaction mixture was quenched with 10% NH₄Cl solution (25 mL) at 0° C. and stirred for 30 min. The volatiles were removed under reduced pressure. To the resulting residue, 10% MeOH in DCM (100 mL) was added. The solid formed was filtered and washed with 10% MeOH in DCM (100 mL). The combined filtrate was concentrated under reduced pressure to afford 3 as gum.

Yield: 5.4 g (66%)

### Step-3:

To the stirred solution of 3 (4.0 g, 31.0 mmol) and DMAP (0.757 g, 6.19 mmol) in DCM (80 mL), were added Et3N (8.63 mL, 61.9 mmol) and tosyl chloride (11.81 g, 61.9 mmol) at 0° C., and the reaction mixture was allowed to stir at room temperature for 18 h. The reaction mixture was quenched with water (25 mL) at 0° C. and extracted with DCM (3×50 mL). The combined organic extract was washed with brine solution (30 mL), dried over anhydrous Na2SO4, filtered and evaporated under reduced pressure. The crude residue, thus obtained, was purified using MPLC (manually packed SiO₂ cartridge 230-400 mesh size; 90% EtOAc in hexanes) to afford 4 as a white solid.

Yield: 3.2 g (36%)

LCMS: Calculated for C₁₃H₁₇NO₄S is 283.3. Observed: 284.2 [M+1]⁺

### Step-4:

To a stirred solution of 4-bromophenol (5, 2.1 g, 12.14 mmol) in DMF (20 mL), were added 4 (3.44 g, 12.14 mmol) and Cs₂CO₃ (7.91 g, 24.28 mmol) at 25° C. The reaction mixture was stirred at 90° C. for 16 h, after which it was cooled to room temperature and filtered through Celite bed. The Celite bed was washed with EtOAc (30 mL). The filtrate was combined, and the combined filtrate washed with cold water (3×20 mL) and brine solution (20 mL), dried over anhydrous Na2SO4, filtered and concentrated under reduced pressure. The crude residue was purified using MPLC (manually packed SiO₂ cartridge, 230-400 mesh size; 90% EtOAc in hexanes) to afford 6 as an off-white solid.

Yield: 3.5 g (91%)

LCMS: Calculated for C₁₂H₁₄BrNO₂ is 284.15. Observed: 284.2 [M]⁺ and 286.2 [M+2]⁺

The trans-geometry of 6 was secured by nOe studies wherein irradiation of the methine α- to Oxygen at 4.8 ppm does not show any enhancement in the resonance corresponding to the proton α- to the carboxamide, and vice versa.

### Step-5:

To a stirred solution of 6 (1.0 g, 3.52 mmol) in 1,4-dioxane (20 mL), were added potassium acetate (0.691 g, 7.04 mmol) and bis(pinacolato)diboron (1.341 g, 5.28 mmol) at room temperature, and the reaction mixture purged with nitrogen for 5 min. To this reaction mixture, was added PdCl₂(dppf) (0.258 g, 0.352 mmol) and the resulting reaction mixture was heated to 90° C. for 16 h. The reaction mixture was cooled to room temperature, diluted with EtOAc (10 mL) and filtered through a Celite pad. The Celite pad was washed with EtOAc (30 mL×2). The combined filtrate was washed with brine solution (15 mL), dried over anhydrous Na2SO4, filtered, and concentrated under reduced pressure. The crude residue thus obtained was purified by using MPLC (manually packed SiO₂ cartridge, 230-400 mesh size; 45% EtOAc in hexanes) to afford 7 as a white solid.

Yield: 820 mg (65%)

LCMS: Calculated for C₁₈H₂₆BNO₄ is 331.22, Observed: 331.9 [M+1]⁺

### Step-6:

To a stirred solution of 8 (500 mg, 1.192 mmol) in ACN (6 mL) and water (3 mL), were added 7 (474 mg, 1.431 mmol) and K₂CO₃ (494 mg, 3.58 mmol) at room temperature. The reaction mixture was purged with nitrogen for 5 min. To this reaction mixture, PdCl₂(dtbpf) (38.9 mg, 0.060 mmol) was added and the resulting reaction mixture heated at 85° C. for 16 h. The reaction mixture was quenched with ice water (15 mL) and extracted with 5% MeOH in DCM (50 mL×3). The combined organic extract was washed with brine solution (15 mL), dried over anhydrous Na2SO4, filtered, and concentrated under reduced pressure. The crude residue, thus obtained, was purified by using MPLC (using manually packed SiO2 cartridge; 230-400 mesh size; 4% MeOH in DCM) to get 9 as a brown solid.

Yield: 220 mg (33%)

LCMS: Calculated for C₃₂H₃₇N₃O₅ is 543.66. Observed: 544.0 [M+1]⁺

### Step-7:

To a stirred solution of 9 (220 mg, 0.397 mmol) in MeOH (40 mL), was added p-toluenesulfonic acid monohydrate (226 mg, 1.190 mmol) at 0° C. The reaction mixture was allowed to room temperature and stirred for 4 h. The reaction mixture was quenched with sat. NaHCO₃ solution (8 mL) at 0° C. The suspension was extracted with DCM (100 mL×3). The combined organic extract was washed with sat. NaHCO₃ solution (2×5 mL), brine solution (10 mL), dried over anhydrous Na2SO4, filtered, and concentrated under reduced pressure. The crude residue, thus obtained, was purified by using reverse phase preparative HPLC (Column: ZOBRAX C18 (21.2×50 mm) 7 μm; Eluents:10 mM ammonium bicarbonate in water and ACN) to obtain Compound 122 as an off-white solid.

Yield: 50 mg (27%)

LCMS: Calculated for C₂₇H₂₉N₃O₄ is 459.55, Observed: 460.0 [M+1]⁺

1H-NMR (400 MHz, DMSO-d6): δ 7.80 (br s, 1H, exchanges with D2O), 7.66-7.62 (m, 4H), 7.51 (d, J=8.00 Hz, 2H), 7.35 (d, J=1.20 Hz, 1H), 6.90 (d, J=8.80 Hz, 2H), 6.84 (d, J=1.20 Hz, 1H), 5.69 (t, J=5.60 Hz, 1H), 5.53 (t, J=5.60 Hz, 1H, exchanges with D2O), 5.37 (d, J=5.60 Hz, 1H, exchanges with D2O), 4.94 (t, J=6.00 Hz, 1H), 4.88 (t, J=6.40 Hz, 1H), 3.87 (t, J=6.00 Hz, 2H), 3.03-3.01 (m, 1H), 2.62-2.51 (m, 5H), 2.30-2.23 (m, 2H), 1.51 (d, J=6.40 Hz, 3H).

SFC purity=100%; tR=2.95 min (Column: Whelk-(R,R); Eluents: 0.5% isopropyl amine in MeOH and CO2) trans-geometry at tail part; single isomer with 100% SFC purity.

### Example A71: Synthesis of Compound 123

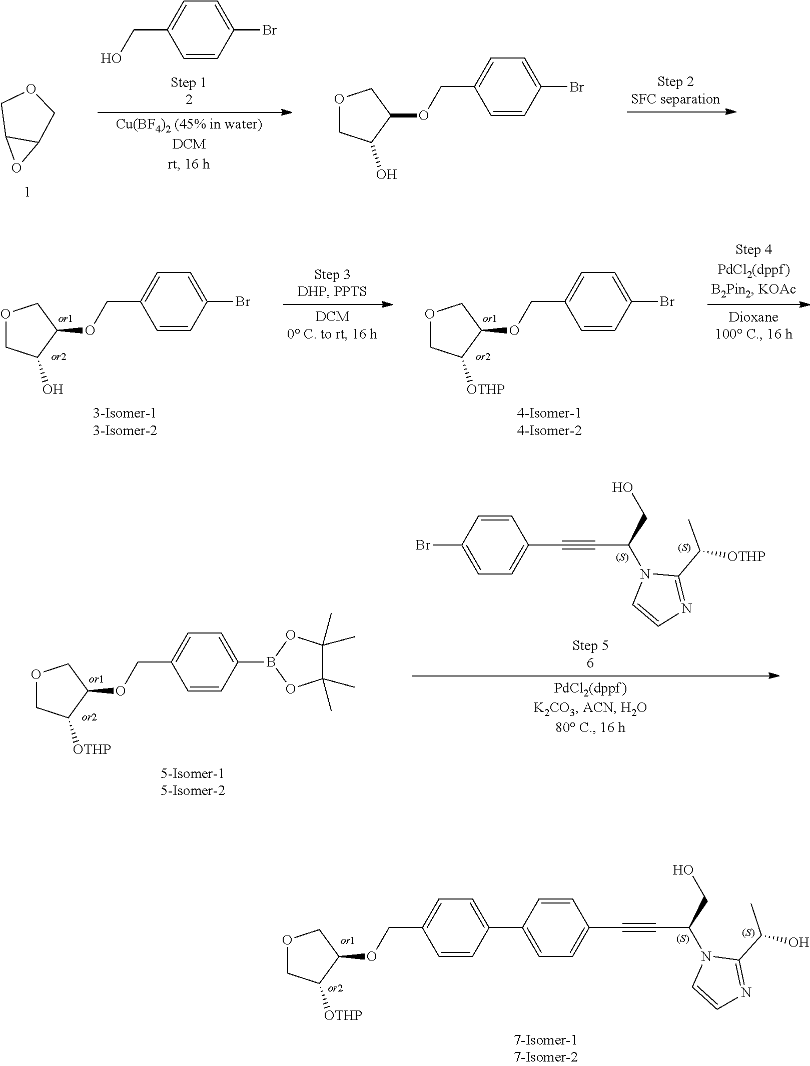

### Step 1:

To a suspension of Copper(II) tetrafluoroborate (45% in water, 4.54 mL, 13.37 mmol) in DCM (250 mL), were added 4-Bromobenzyl alcohol (2,25 g, 134 mmol) and 3,4-epoxytetrahydrofuran (1, 19.18 mL, 267 mmol) at room temperature. The reaction was stirred at room temperature for 16 h. The reaction mixture was quenched with water (200 mL) and extracted with DCM (200 mL×2). The combined organic layer was washed with brine (50 mL), dried over anhydrous Na₂SO₄, filtered and concentrated under reduced pressure. The crude product was purified by using MPLC (manually packed cartridge; SiO₂ 100-200 mesh; 70% EtOAc in hexanes) to afford (±)-3 as an off-white solid. Yield: 3.75 g (10%)

### Step 2:

18.5 g of (±)-3 was separated by using SFC (Column: I Amylose A (250×30) mm, 5 μm; Eluents: CO2: Methanol [85:15]) to get 3_Isomer-1 (retention time: 3.76 min) and 3_Isomer-2 (retention time: 4.60 min) as an off-white solids. Yield: 3_Isomer-1: 8 g and 3_Isomer-2: 6.9 g. SFC purity=3_Isomer-1 (t_{R}=3.74 min): 99.7% and 3_Isomer-2 (t_{R}=4.55 min): 97.2%. Both the isomers (3-Isomer-1 and 3-Isomer-2) were taken independently for further conversion.

### Step-3: Using 3-Isomer-1:

To a stirred solution of 3-Isomer-1 (3.0 g, 10.98 mmol) in DCM (30 mL), was added 3,4-dihydro-2H-pyran (1.848 g, 21.97 mmol) followed by pyridinium p-toluenesulfonate (0.055 g, 0.220 mmol) at room temperature. The reaction mixture was stirred at room temperature for 16 h. The reaction was quenched by the addition of a saturated solution of sodium bicarbonate (100 mL) and extracted with DCM (100 mL×2). The combined organic layer was washed with brine (50 mL), dried over anhydrous Na₂SO₄, filtered and concentrated under reduced pressure. The crude product was purified by using MPLC (manually packed cartridge, SiO₂ 230-400 mesh; 10% EtOAc in hexanes) to afford 4-Isomer-1 as a colorless oil. Yield: 3.8 g (92%)

### Step 4: Using 4-Isomer-1

To a solution of 4-Isomer-1 (3.8 g, 10.64 mmol) and bis(pinacolato)diboron (4.05 g, 15.96 mmol) in dioxane (38 mL) was added potassium acetate (3.13 g, 31.9 mmol). The reaction mixture was degassed for 5 min and PdCl2(dppf) (0.778 g, 1.064 mmol) was added. The mixture was heated at 100° C. for 16 h. The reaction mixture was quenched with water (100 mL) and extracted with EtOAc (100 mL×2). The combined organic layer was washed with brine (50 mL), dried over anhydrous Na₂SO₄, filtered and concentrated under reduced pressure to get the crude product. The crude residue was purified by MPLC (manually packed cartridge, SiO₂ 230-400 mesh; 10% EtOAc in hexanes) to obtain 5-Isomer-1 as an off-white solid. Yield: 2.9 g (64%)

### Step 5: Using 5-Isomer-1

To a stirred solution of 5-Isomer-1 (1.018 g, 2.52 mmol) in water (5 mL) and acetonitrile (5 mL), were added 6 (0.88 g, 2.099 mmol) and potassium carbonate (0.870 g, 6.30 mmol) at room temperature. The reaction mixture was purged with nitrogen for 5 min. To this reaction mixture, PdCl2(dtbpf) (0.137 g, 0.210 mmol)) was added and the purging continued for another 2 min. The resulting reaction mixture was stirred at 80° C. for 16 h. The reaction mixture was quenched with water (50 mL) and extracted with 10% MeOH in DCM (50 mL×2). The combined organic layer was washed with brine (30 mL), dried over anhydrous Na₂SO₄, filtered and concentrated under reduced pressure to get the crude product. The crude product was purified by MPLC (manually packed cartridge, SiO₂ 230-400 mesh; 5% MeOH in DCM) to obtain 7-Isomer-1 as a brown color solid. Yield: 730 mg (52%). LC-MS: Calculated for C36H44N2O7 is 616.75, Observed: 617.2 [M+1]⁺

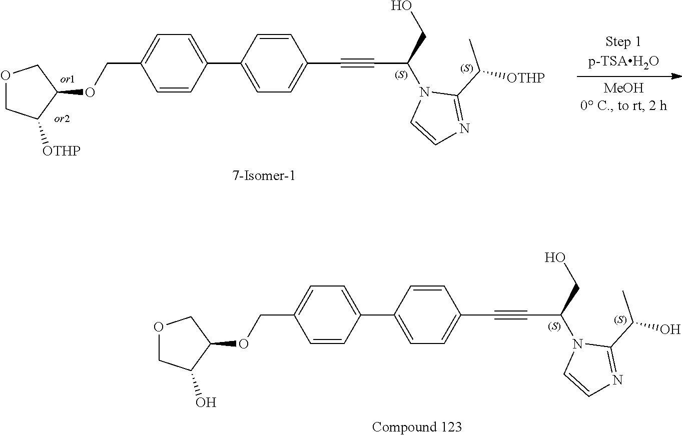

### Step 1:

To a stirred solution of 1 (0.4 g, 0.649 mmol) in MeOH (10 mL), was added p-toluenesulfonic acid monohydrate (0.370 g, 1.946 mmol) at 0° C., and the reaction mixture stirred at room temperature for 2 h. The volatiles in the reaction were concentrated under reduced pressure and the crude residue basified with sat. NaHCO3 solution (15 mL). The aqueous layer was extracted with 10% MeOH in DCM (15 mL×2). The combined organic extract was dried over anhydrous Na2SO4, filtered and concentrated under reduced pressure. The crude residue was purified by preparative HPLC (Column: X SELECT C18 (19×150 mm) 5 μm; Eluents: 10 mM ammonium bicarbonate in water and acetonitrile) to obtain Compound 123 as a white solid. Yield: 60 mg (20%) LC-MS: Calculated for C₂₆H₂₈N₂O₅ is 448.52, Observed: 449.2 [M+1]⁺ 1H-NMR (400 MHz, DMSO-d6): δ 7.72-7.68 (m, 4H), 7.55 (d, J=8.40 Hz, 2H), 7.43 (d, J=8.40 Hz, 2H), 7.36 (d, J 1.20 Hz, 1H), 6.84 (d, J=0.80 Hz, 1H), 5.70 (t, J=6.00 Hz, 1H), 5.54 (t, J=5.60 Hz, 1H, exchanges with D₂O), 5.38 (d, J=5.60 Hz, 1H, exchanges with D₂O), 5.16 (d, J=4.00 Hz, 1H, exchanges with D₂O), 4.96-4.93 (m, 1H), 4.58 (q, J=12.00 Hz, 2H), 4.21 (t, J=3.60 Hz, 1H), 3.91-3.81 (m, 5H), 3.72 (d, J=−9.60 Hz, 1H), 3.53 (dd, J=1.60, 9.20 Hz, 1H), 1.51 (d, J=6.40 Hz, 3H). SFC: 92.9%; tR=4.86 min (Column: LUX-Amylose-1; eluents: CO2 and 0.5% isopropyl amine in MeOH). Single isomer with SFC purity 92.9%

### Example A72: Synthesis of Compound 124

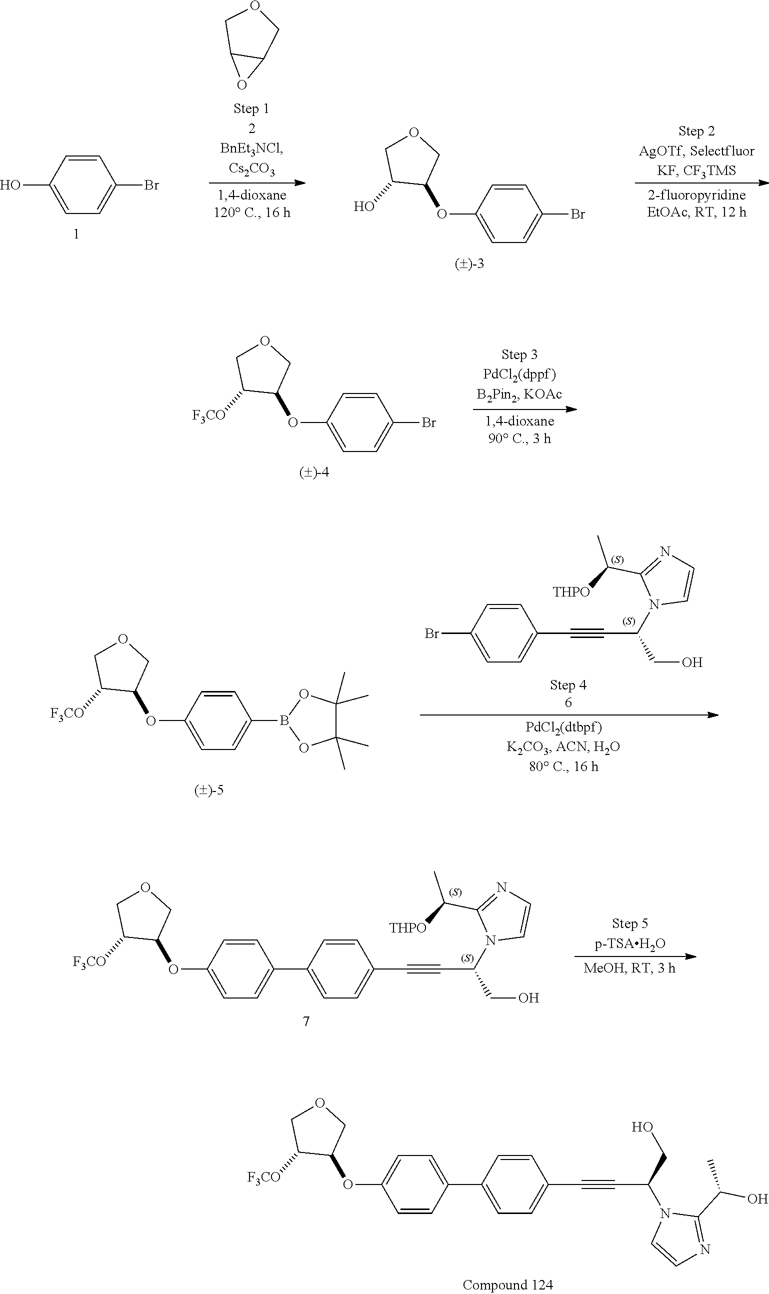

### Step-1:

To a solution of 4-bromophenol (1, 25 g, 145 mmol) in 1,4-dioxane (500 mL), cesium carbonate (70.6 g, 217 mmol), 3,4-epoxytetrahydrofuran (2, 10.37 mL, 145 mmol) and benzyltriethylammonium chloride (6.58 g, 28.9 mmol) were added at room temperature. After complete addition, the resulting reaction mixture was stirred at 120° C. for 16 h. The reaction was cooled to room temperature, filtered through Celite bed, and washed with EtOAc (2×250 mL). The filtrate was concentrated under reduced pressure to afford the crude residue as a pale-yellow gum. The crude residue was purified by MPLC (manually packed SiO₂ cartridge 230-400 mesh size; 35-40% EtOAc in hexanes) to afford (±)-3 as an off-white solid.

Yield: 22.5 g (52%)

LC MS: Calculated for C10H11BrO3 is 257.99 (exact mass), Observed: 258.0 [M]⁺ and 260.0 [M+2]⁺

### Step-2:

A 50 mL two neck RBF was equipped with a stirring bar and nitrogen balloon was charged with silver trifluoromethanesulfonate (4.02 g, 15.63 mmol), selectfluor (2.77 g, 7.82 mmol), potassium fluoride (1.211 g, 20.84 mmol), and (±)-3 (1.5 g, 5.21 mmol) successively under nitrogen environment. Then, EtOAc (15 mL), 2-fluoropyridine (2.032 mL, 15.63 mmol) and trimethyl(trifluoromethyl)silane (2.310 mL, 15.63 mmol) were added under nitrogen atmosphere via syringe. The resulting reaction mixture was stirred at room temperature for 12 h. The reaction mixture was diluted with EtOAc (20 mL) and filtered through a plug of silica, eluted with EtOAc (2×15 mL). The filtrate was concentrated and obtained crude was purified by MPLC (manually packed cartridge, SiO2 230-400 mesh size; 0-10% EtOAc in hexanes) to afford (±)-4 as colorless liquid.

Note: Three more batches were performed on 1.5 g scale. All the batches mixed for work up and purified to get 1.6 g of product

Yield: 1.6 g

Reference: Org. Lett. 2015, 17, 5048.

### Step-3:

To a stirred solution of (±)-4 (1.6 g, 4.89 mmol), bis(pinacolato)diboron (1.863 g, 7.34 mmol) in 1,4-dioxane (15 mL) was added potassium acetate (0.960 g, 9.78 mmol). The resulting reaction mixture was purged with nitrogen for 5 min, PdCl₂(dppf) (0.286 g, 0.391 mmol) added and the resulting reaction mixture heated to 90° C. for 3 h. The reaction mixture was diluted with water (8 mL) and extracted with EtOAc (16 mL×3). The combined organic extract was washed with brine (8 mL), dried over anhydrous sodium sulphate, filtered, and concentrated under reduced pressure to afford crude, which was further purified by MPLC (manually packed cartridge, SiO2 230-400 mesh size; 0-15% EtOAc in hexanes) to afford (±)-5 as a white solid.

Yield: 900 mg (44%)

### Step-4:

To a stirred solution of 6 (400 mg, 0.906 mmol), in acetonitrile (2 mL) and water (2 mL), were added K₂CO₃ (250 mg, 1.812 mmol) and (±)-5 (509 mg, 1.359 mmol). The resulting reaction mixture was purged with nitrogen for two minutes. To this reaction mixture, PdCl2(dtbpf) (59.1 mg, 0.091 mmol) was added at room temperature. The resulting reaction mixture was heated to 80° C. and stirred for 16 h. The reaction was quenched with ice-cold water (8 mL) and extracted with 5% MeOH in DCM (16 mL×3). The combined organic extract was washed with brine (8 mL), dried over anhydrous sodium sulphate, filtered, and concentrated under reduced pressure to afford a crude residue, which was purified by MPLC (manually packed cartridge, SiO2 230-400 mesh size; 0-5% MeOH in DCM) to afford 7 as a brown sticky solid. One more batch was carried out on 400 mg of (±)-5. Both the batches were mixed for work up and purification. The yield mentioned is based on the total yield obtained (380 mg) for combined batches.

Yield: 380 mg

LC MS: Calculated for C31H33F3N2O6 is 586.61, Observed: 587.3 [M+1]⁺

### Step-5:

To the stirred solution of 7 (125 mg, 0.213 mmol) in MeOH (30 mL), was added p-toluenesulfonic acid monohydrate (122 mg, 0.639 mmol) at 0° C. The resulting reaction mixture was allowed to stir at room temperature for 3 h. The reaction was quenched with sat. NaHCO₃ solution (5 mL) at 0° C. and extracted with DCM (100 mL×3). The combined organic layer was washed with sat. NaHCO₃ solution (2×5 mL), brine solution (8 mL), dried over anhydrous sodium sulphate, filtered, and concentrated to afford crude residue, which was purified by preparative HPLC to afford Compound 124 as a white solid.

Yield: 45 mg (14%)

LC MS: Calculated for C26H25F3N2O5 is 502.17, Observed: 503.2 [M+1]⁺

The final compound was a mixture of diastereomers; racemic at tail.

### Example A73: Synthesis of Compounds 125 and 126

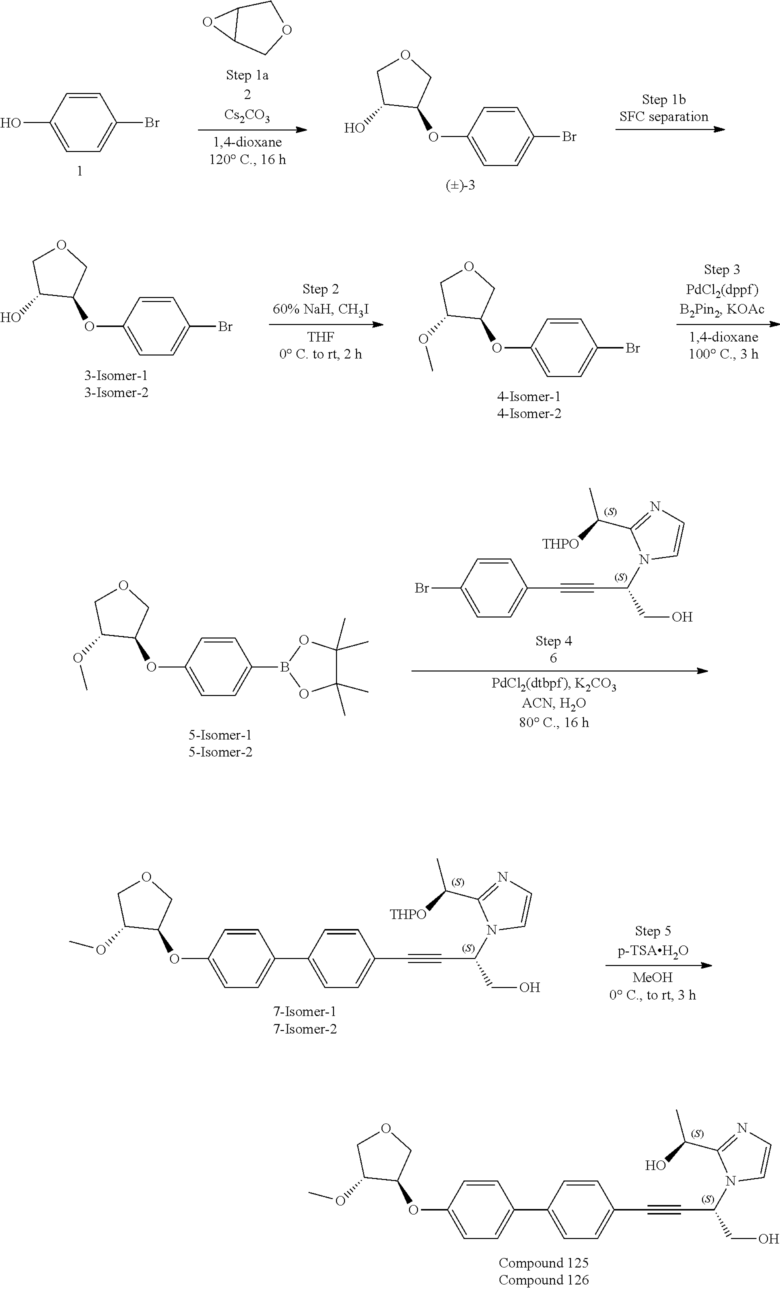

### Step-1a:

To a stirred solution of 4-bromophenol (1, 20 g, 116 mmol) in 1,4-dioxane (200 mL), were added 3,4-epoxytetrahydrofuran (2, 9.95 g, 116 mmol), Cs₂CO₃ (56.5 g, 173 mmol) and benzyltriethylammonium chloride (2.63 g, 11.56 mmol) under nitrogen at room temperature. The reaction mixture was heated at 120° C. for 16 h. After completion of reaction, the inorganic solids were filtered through Celite pad and washed with EtOAc (3×150 mL). The filtrate was concentrated under reduced pressure and the resulting crude residue purified by MPLC (using manually packed SiO₂ cartridge, 100-200 mesh; 22% EtOAc in hexanes) to afford (±)-3 as a yellow colored sticky solid.

Yield=21.1 g (67%)

### Step-1b:

The enantiomers of (±)-3 (21.1 g) were separated by SFC (Column: LUX-A1 (250*30) mm, 5 μm; Eluents: CO₂:0.5% isopropylamine in methanol [70:30]). The fractions were concentrated under reduced pressure to afford 3-Isomer-1 (tR=2.995 min) and 3-Isomer-2 (tR=4.38 min).

Yield=3-Isomer-1: 6.8 g (ee=100%) and 3-Isomer-2: 6.1 g (ee=100%)

Both isomers were taken individually for further conversions.

### Compound 125:

### Step-2:

To a stirred solution of 3-Isomer-1 (6.7 g, 25.9 mmol) in THF (100 mL), was added NaH (60% in paraffin oil; 2.069 g, 51.7 mmol) in portions under nitrogen at 0° C. After stirring for 30 min at same temperature, iodomethane (1.607 mL, 25.7 mmol) was added and the reaction mixture stirred at room temperature for 2 h. After completion of reaction, the reaction mixture was cooled to 0° C., quenched with ice cold water (100 mL) and extracted with EtOAc (2×100 mL). The combined organic layer was dried over anhydrous Na₂SO₄, filtered and concentrated under reduced pressure to afford 4-Isomer-1 as a pale-yellow liquid.

Yield=7 g (91%)

### Step-3:

To a solution of 4-Isomer-1 (7 g, 25.6 mmol) in 1,4-dioxane (100 mL), were added potassium acetate (7.55 g, 77 mmol) and bis(pinacolato)diboron (9.76 g, 38.4 mmol) at room temperature. The resulting mixture was purged with nitrogen for 5 min. To this reaction mixture, PdCl₂(dppf) (1.875 g, 2.56 mmol) was added and the purging continued for another 2 min. The reaction mixture was then heated at 100° C. for 3 h. After completion of reaction, the inorganic solids were filtered through Celite pad and washed with EtOAc (150 mL). The combined filtrate was concentrated under reduced pressure. The crude residue was purified by MPLC (using manually packed SiO₂ cartridge, 100-200 mesh size; 10-25% EtOAc in hexanes) to afford 5-Isomer-1 as a brown gum.

Yield: 8 g (78%)

### Step-4:

To a stirred solution of 5-Isomer-1 (2.63 g, 8.23 mmol) in acetonitrile (20 mL) and water (20 mL), were added 6 (2.3 g, 5.49 mmol) and K₂CO₃ (2.274 g, 16.46 mmol) at room temperature. The reaction mixture was purged with nitrogen for 5 min. To this reaction mixture, PdCl₂(dtbpf) (0.357 g, 0.549 mmol) was added and purging with nitrogen was continued for another 2 min. The resulting reaction mixture was stirred at 80° C. for 16 h. The reaction mixture was quenched with water (50 mL), extracted with 10% MeOH in DCM (50 mL×2). The combined organic layer was washed with brine solution (30 mL), dried over anhydrous Na₂SO₄, filtered and concentrated under reduced pressure. The resulting crude product was purified by MPLC (manually packed SiO₂ cartridge, 230-400 mesh size; 5% MeOH in DCM) to afford 7-Isomer-1 as pale-brown solid.

Yield: 1.4 g (44%)

LC-MS: Calculated for C₃₁H₃₆N₂O₆ is 532.64, Observed: 533.0 [M+1]⁺

### Step-5:

To a stirred solution of 7-Isomer-1 (200 mg, 0.375 mmol) in MeOH (10 mL), was added p-toluene sulfonic acid monohydrate (214 mg, 1.126 mmol) at 0° C. The reaction mixture was warmed to room temperature and stirred for 3 h. The volatiles were evaporated under reduced pressure, the resulting residue basified with 10% NaHCO₃ solution (30 mL) and extracted with 10% MeOH in DCM (50 mL×2). The combined organic layer was washed with brine (30 mL), dried over anhydrous Na₂SO₄, filtered and concentrated under reduced pressure. The crude was purified by reversed phase preparative HPLC (10 mM ammonium bicarbonate in water and acetonitrile) to afford Compound 125 as an off-white solid.

Yield=90 mg (53%)

LC-MS: Calculated for C₂₆H₂₈N₂O₅ is 448.52, Observed: 449.3 [M+1]⁺

1H-NMR (400 MHz, DMSO-d6): δ 7.67 (d, J=8.40 Hz, 4H), 7.52 (d, J=8.40 Hz, 2H), 7.36 (app d, J=1.20 Hz, 1H), 7.08 (d, J=8.40 Hz, 2H), 6.84 (s, 1H), 5.68 (t, 1H), 5.53 (t, J=5.60 Hz, 1H, exchanges with D2O), 5.37 (d, J=6.00 Hz, 1H, exchanges with D2O), 4.94-4.93 (m, 2H), 4.05-4.02 (m, 2H), 3.95-3.91 (m, 1H), 3.87 (t, J=5.60 Hz, 2H), 3.78-3.75 (m, 2H), 3.32 (s, 3H, merges with solvent water), 1.51 (d, J=6.80 Hz, 3H). Unknown stereochemistry at tail part, single isomer with SFC purity=94.8% (l-Cellulose-Z_0.5% IPAm in ACN_MeOH tR=5.33 min)

### Compound 126

### Step-2:

To a stirred solution of 3-Isomer-2 (5.9 g, 22.77 mmol) in THE (100 mL), was added NaH (60% in paraffin oil; 1.822 g, 45.5 mmol) in portions at 0° C. under nitrogen atmosphere. After stirring for 30 min at same temperature, iodomethane (1.418 mL, 22.77 mmol) was added and the reaction mixture stirred at room temperature for 2 h. After completion of reaction, the reaction mixture was cooled to 0° C., quenched with ice cold water (100 mL) and extracted with EtOAc (2×150 mL) dried over anhydrous Na₂SO₄, filtered and concentrated under reduced pressure to afford 4-Isomer-2 as a pale-yellow liquid.

Yield=6.2 g (95%)

### Step-3:

To a solution of 4-Isomer-2 (6.2 g, 22.7 mmol) in 1,4-dioxane (100 mL), were added potassium acetate (6.68 g, 68.1 mmol) and bis(pinacolato)diboron (8.65 g, 34.1 mmol) at room temperature. The resulting mixture was purged with nitrogen for 5 min. To this reaction mixture, PdCl₂(dppf) (0.830 g, 1.135 mmol) was added and the purging continued for another 2 min. The reaction mixture was heated at 100° C. for 3 h. After completion of the reaction, the inorganic solids were filtered through Celite pad and washed with EtOAc (150 mL). The combined filtrate was concentrated under reduced pressure to afford a crude residue. The crude residue was purified by MPLC (using manually packed SiO₂ cartridge, 100-200 mesh size; 10-25% EtOAc in hexanes) to afford 5-Isomer-2 as yellow gum.

Yield: 7 g (87%)

### Step-4:

To a stirred solution of 5-Isomer-2 (2.63 g, 8.23 mmol) in acetonitrile (20 mL) and water (5 mL), were added 6 (2.3 g, 5.49 mmol) and K₂CO₃ (2.274 g, 16.46 mmol) at room temperature under nitrogen. The reaction mixture was purging with nitrogen for 5 min. To this reaction mixture, PdCl₂(dtbpf) (0.179 g, 0.274 mmol) was added and purging with nitrogen continued for another 2 min. The resulting reaction mixture was stirred at 80° C. for 16 h. The reaction mixture was quenched with water (50 mL), extracted with 10% MeOH in DCM (50 mL×2). The combined organic layer was washed with brine (30 mL), dried over anhydrous Na₂SO₄, filtered and concentrated under reduced pressure. The resulting crude product was purified by MPLC (manually packed SiO₂ cartridge, 230-400 mesh size; 5% MeOH in DCM) to afford 7-Isomer-2 as a pale-yellow solid.

Yield: 1.6 g (51%)

LC-MS: Calculated for C₃₁H₃₆N₂O₆ is 532.6, Observed: 533.0 [M+1]⁺

### Step-5:

To a stirred solution of 7-Isomer-2 (200 mg, 0.375 mmol) in MeOH (5 mL), was added p-toluenesulfonic acid monohydrate (214 mg, 1.126 mmol) at 0° C. The reaction mixture was warmed to room temperature and stirred for 3 h. The volatiles were evaporated under reduced pressure. The resulting residue was basified with 10% NaHCO₃ solution (20 mL) and extracted with 10% MeOH in DCM (50 mL×2). The combined organic layer was washed with brine (30 mL), dried over anhydrous Na₂SO₄, filtered and concentrated under reduced pressure. The crude thus obtained was purified by reversed phase preparative HPLC (Column: Shimpack C18 (150*20 mm) 5 μm, Eluents: 10 mM ammonium bicarbonate in water and acetonitrile) to afford Compound 126 as an off-white solid.

Yield=53 mg (31%)

LC-MS: Calculated for C₂₆H₂₈N₂O₅ is 448.52, Observed: 449.0[M+1]⁺

1H-NMR (400 MHz, DMSO-d6): δ 7.67 (d, J=8.40 Hz, 4H), 7.52 (d, J=8.40 Hz, 2H), 7.36 (app d, J=1.20 Hz, 1H), 7.08 (d, J=8.80 Hz, 2H), 6.84 (app d, J=1.20 Hz, 1H), 5.69 (t, J=6.00 Hz, 1H), 5.54 (br s, 1H, exchanges with D2O), 5.37 (d, J=5.60 Hz, 1H, exchanges with D2O), 4.95-4.93 (m, 2H), 4.05-4.00 (m, 2H), 4.00-3.91 (m, 1H), 3.87 (br s, 2H), 3.78-3.74 (m, 2H), 3.32 (s, 3H, merges with solvent water), 1.51 (d, J=6.80 Hz, 3H).

Unknown stereochemistry at tail part, single isomer with SFC purity=98.7% (l-Cellulose-Z_0.5% IPAm in ACN_MeOH tR=4.90 min)

### Example A74: Synthesis of Compound 127

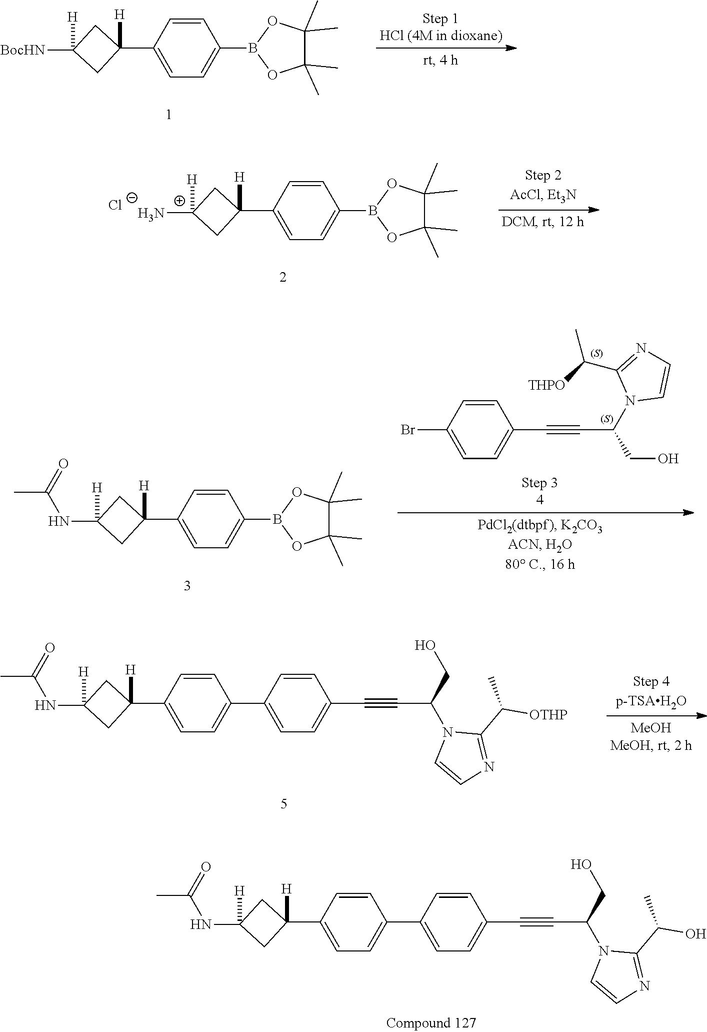

### Step 1:

To a stirred solution of 1 (2.5 g, 6.70 mmol) in DCM (20 mL), was added HCl (4 M in dioxane, 6.70 mL, 26.8 mmol dropwise at 0° C. The reaction mixture was stirred for 4 h at room temperature. The volatiles present in the reaction mixture were removed under reduced pressure. The crude residue was co-distilled with toluene (2×20 mL) and triturated with 50% EtOAc in hexane (30 mL) to obtain a colorless solid. The obtained solid was filtered and dried under vacuum to afford 2 as an off-white solid.

LCMS: Calculated for C₁₆H₂₅BNO₂⁺ is 274.2, Observed: 274.1 [M]⁺

Yield: 1.8 g (80%)

### Step 2:

To a stirred solution of 2 (1.7 g, 5.49 mmol) in DCM (20 mL), was added triethylamine (2.3 mL, 16.47 mmol) and acetyl chloride (0.59 mL, 8.24 mmol) at 0° C. and the resulting reaction mixture was stirred at room temperature for 12 h. The reaction mixture was quenched with 10% NaHCO₃ and extracted with DCM (2×30 mL). The combined organic layer was dried over anhydrous Na₂SO₄, filtered and concentrated under reduced pressure. The crude residue thus obtained was purified by using MPLC (manually packed SiO₂ cartridge, 100-200 mesh size; 80% EtOAc in hexanes) to get 3 (1.65 g, 4.24 mmol, 77% yield) as a solid.

LCMS: Calculated for C₁₈H₂₆BNO₃ is 315.20, Observed: 316.2 [M+1]⁺

### Step 3:

To a stirred solution of 3 (0.301 g, 0.954 mmol) and 4 (0.4 g, 0.954 mmol) in a mixture of acetonitrile (3 mL) and water (3 mL), was added potassium carbonate (0.225 g, 1.62 mmol) and the reaction mixture purged with nitrogen for 5 min. Subsequently, PdCl₂(dtbpf) (48.3 mg, 0.074 mmol) was added and the reaction stirred at 80° C. for 16 h. The reaction mixture was quenched with water (10 mL) and extracted with 10% MeOH in DCM (20 mL×2). The combined organic layer was washed with brine (10 mL), dried over anhydrous Na₂SO₄, filtered and concentrated under reduced pressure. The crude residue thus obtained was purified by using MPLC (manually packed SiO₂ cartridge, 230-400 mesh size; 5% MeOH in DCM) to afford 5 as a brown solid.

Yield: 0.27 g (53%)

LCMS: Calculated for C₃₂H₃₇N₃O₄ is 527.28, Observed: 528.3 [M+1]⁺

### Step 4:

To a stirred solution of 5 (0.25 g, 0.474 mmol) in MeOH (10 mL), was added p-toluenesulfonic acid monohydrate (0.270 g, 1.421 mmol) at 0° C. and the resulting reaction mixture stirred at room temperature for 2 h. The volatiles were removed under reduced pressure. The resulting residue was dissolved in 10% MeOH in DCM (100 mL), 10% NaHCO₃ solution (3×50 mL), dried over anhydrous sodium sulphate, filtered and concentrated under reduced pressure. The crude residue thus obtained was purified by using MPLC (manually packed SiO₂ cartridge, 230-400 mesh size; 6% MeOH in DCM) to afford Compound 127 (0.102 g, 0.229 mmol, 48.3% yield) as a colorless solid.

Yield: 0.102 g (48%)

LCMS: Calculated for C₂₇H₂₉N₃O₃ is 443.55, Observed: 444.2 [M+1]⁺

1H-NMR (400 MHz, DMSO-d6): δ 8.30 (d, J=6.80 Hz, 1H), 7.70 (d, J=8.40 Hz, 2H), 7.66 (d, J=8.00 Hz, 2H), 7.54 (d, J=8.00 Hz, 2H), 7.39 (d, J=8.00 Hz, 2H), 7.36 (d, J=1.20 Hz, 1H), 6.85 (d, J=0.80 Hz, 1H), 5.70 (t, J=5.60 Hz, 1H), 5.54 (t, J=5.60 Hz, 1H, exchanges with D2O), 5.38 (d, J=5.60 Hz, 1H, exchanges with D2O), 4.96-4.93 (m, 1H), 4.33-4.27 (m, 1H), 3.87 (t, J=6.00 Hz, 2H), 3.59-3.56 (m, 1H), 2.41-2.34 (m, 4H), 1.82 (s, 3H), 1.51 (d, J=6.40 Hz, 3H).

SFC: 96.9%; tR=6.82 min (Column: LUX-I-Amylose 3; Eluents: 0.5% isopropyl amine in mixture of ACN, MeOH and CO2) trans-geometry at tail; SFC purity=96.9%

### Example A75: Synthesis of Compound 128

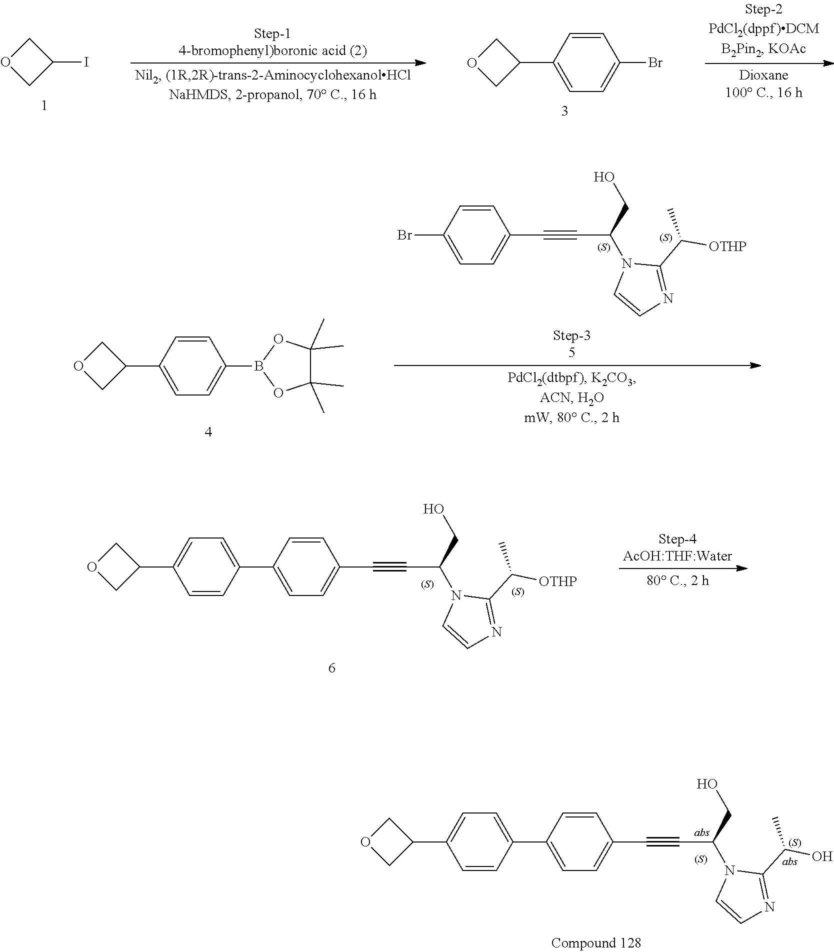

### Step-1:

A stirred solution of nickel(II) iodide (0.467 g, 1.494 mmol) and (1R,2R)-trans-2-aminocyclohexanol hydrochloride (0.227 g, 1.494 mmol) in 2-propanol (30 mL) was purged with nitrogen for 5 min. Then, sodium bis(trimethylsilyl)amide (2 M in THF, 14.94 mL, 29.9 mmol), (4-bromophenyl)boronic acid (2, 3 g, 14.94 mmol) and 3-iodooxetane (1, 2.75 g, 14.94 mmol) were added and the resulting reaction mixture stirred at 70° C. for 16 h. The reaction was quenched with water (100 mL) and extracted with EtOAc (2×100 mL). The combined organic layer was dried over anhydrous Na₂SO₄, filtered and concentrated under reduced pressure. The resulting crude residue was purified by using MPLC (manually packed SiO₂ cartridge, 100-200 mesh size; 12% EtOAc in hexanes) to obtain 3 as a colorless gum.

Yield=2 g (56%)

LCMS: Calculated for C₉H₉BrO is 213.07, Observed: Desired molecular ion not observed.

Reference: J. Am. Chem. Soc., 2006, 128, 5360

### Step 2:

To a stirred solution of 3 (1.5 g, 7.04 mmol) in dioxane (30 mL), were added potassium acetate (2.073 g, 21.12 mmol) and bis(pinacolato)diboron (2.68 g, 10.56 mmol) at room temperature. The resulting reaction mixture was purged with nitrogen for 5 min. To this reaction mixture, PdCl₂(dppf)·DCM (0.309 g, 0.422 mmol) was added and stirred at 100° C. for 16 h. The reaction mixture was cooled to room temperature, filtered through a Celite pad and washed with EtOAc (100 mL). The combined filtrate was concentrated under reduced pressure. The resulting crude residue was purified by using MPLC (manually packed SiO₂ cartridge, 100-200 mesh size; 10% EtOAc in hexanes) to obtain 4 as an off-white solid.

Yield: 1.2 g (64%)

LCMS: Calculated for C₁₅H₂₁BO₃ is 260.14, Observed: 261.3 [M+1]⁺

### Step 3:

To a solution of 5 (500 mg, 1.192 mmol) in acetonitrile (5 mL) and water (5 mL), were added 4 (465 mg, 1.789 mmol) and K₂CO₃ (494 mg, 3.58 mmol) at room temperature and the resulting mixture purged with nitrogen for 5 min. To this reaction mixture, PdCl₂(dtbpf) (0.075 g, 0.11 mmol) was added and irradiated at 80° C. for 2 h in a microwave reactor. The reaction mixture was quenched with water (50 mL) and extracted with 10% MeOH in DCM (2×50 mL). The combined organic layer was dried over anhydrous Na₂SO₄, filtered and concentrated under reduced pressure. The resulting crude residue was purified by MPLC (using manually packed SiO₂ cartridge, 100-200 mesh size; 4% MeOH in DCM) to obtain 6 as an off-white solid.

Yield: 400 mg (64%)

LCMS: Calculated for C₂₉H₃₂N₂O₄ is 472.58, Observed: 473.2 [M+1]⁺

### Step 4:

A solution of 6 (200 mg, 0.423 mmol) in acetic acid (4 mL):THF (2 mL): water (1 mL) was stirred at 80° C. for 2 h. The reaction was quenched with saturated NaHCO₃ solution (30 mL) and extracted with 10% MeOH in DCM (2×30 mL). The combined organic layer was washed with water (30 mL), brine (30 mL), dried over sodium sulfate, filtered and concentrated under reduced pressure. The crude residue, thus obtained, was purified by using MPLC (manually packed cartridge; SiO₂ 100-200 mesh; 4% MeOH in DCM) to obtain Compound 128 as an off-white solid.

Yield: 40 mg (23%)

LCMS: Calculated for C₂₄H₂₄N₂O₃ is 388.47, Observed: 389.3 [M+1]⁺

1H-NMR (400 MHz, DMSO-d6): δ 7.71 (d, J=7.60 Hz, 4H), 7.55 (d, J=8.40 Hz, 2H), 7.51 (d, J=8.00 Hz, 2H), 7.36 (d, J=1.20 Hz, 1H), 6.84 (d, J=1.20 Hz, 1H), 5.68 (t, J=6.0 Hz, 1H), 5.54 (t, J=6.00 Hz, 1H exchanges with D₂O), 5.38 (d, J=5.60 Hz, 1H exchanges with D₂O), 4.99-4.91 (m, 3H), 4.65 (t, J=6.00 Hz, 2H), 4.34-4.27 (m, 1H), 3.87 (t, J=6.00 Hz, 2H), 1.51 (d, J=6.40, 3H).

SFC: 95.2%; tR=4.60 min (Column: LUX-I-Amylose 3; Eluents: 0.5% isopropyl amine in MeOH)

### Example A76: Synthesis of Compound 129

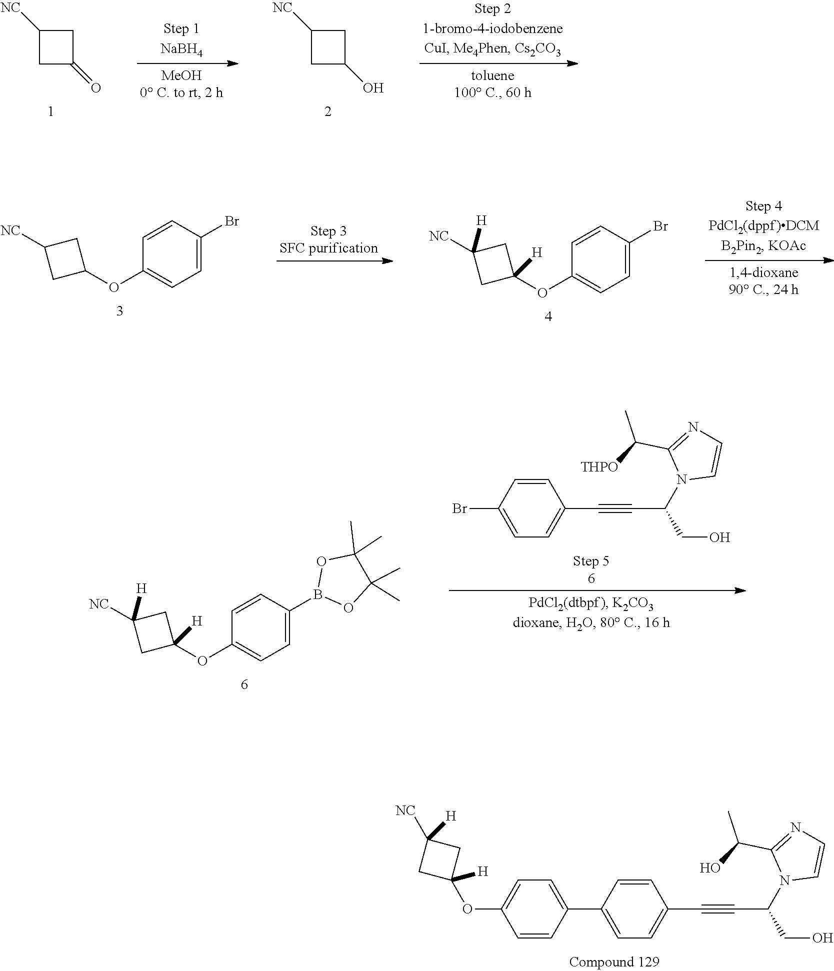

### Step 1:

To a stirred solution of 3-cyanocyclobutanone (1, 5 g, 52.6 mmol) in MeOH (50 mL), was added sodium borohydride (1.193 g, 31.5 mmol) over a period of 10 min at 0° C., and the reaction mixture stirred at 25° C. for 2 h. The reaction mixture was cooled to 0° C., quenched with ice cold water (5 mL) and stirred for 30 min. This was concentrated under reduced pressure. The crude residue, thus obtained was dissolved in 10% MeOH in DCM (100 mL), washed with water (5 mL), brine solution (5 mL), dried over anhydrous Na₂SO₄, filtered and concentrated under reduced pressure to afford 2 as a pale-brown semi-solid. The crude product was taken to the next step without any purification.

Yield: 5 g (crude product weight)

### Step 2:

To a stirred solution of 3,4,7,8-tetramethyl-1,10-phenanthroline (Me4Phen, 0.835 g, 3.53 mmol) and CuI (0.673 g, 3.53 mmol) in toluene (100 mL), were added 1-bromo-4-iodobenzene (10 g, 35.3 mmol), cesium carbonate (17.28 g, 53.0 mmol) and 2 (5.15 g, 53.0 mmol) at 25° C., and a stream of nitrogen gas bubbled through the mixture for 10 min. The reaction was heated to 100° C. for 60 h. The reaction mixture was cooled to 25° C., diluted with EtOAc (50 mL), filtered through the Celite pad. The pad was washed with EtOAc (50 mL), the filtrate combined and concentrated under reduced pressure. The crude residue, thus obtained, was purified by using MPLC (manually packed SiO₂ cartridge, 230-400 mesh; 20% EtOAc in hexanes) to afford 3 (mixture of cis- and trans-) as a pale-yellow semi solid.

Yield: 2 g (21%)

### Step 3:

2.7 g of 3 (mixture of cis- and trans- compounds) was purified by using SFC purification (column: Lux A3-(250×30) mm, 5 μm; eluents: CO₂ and 0.2% formic acid in isopropyl alcohol:acetonitrile (90:10)) to afford 1.7 g of 4 as a pale-yellow solid.

Yield: 1.7 g

SFC: 100%; tR=2.35 min (column: LUX-I-Amylose 3; eluents: CO₂ and 0.2% isopropyl amine in acetonitrile).

The identity of cis-geometry of 4 was established through the nOe enhancement observed at 3.09 ppm (methine α- to nitrile) upon irradiation of the other methine at 4.68 ppm (methine α- to phenoxy).

### Step 4:

To a stirred solution of 4 (0.2 g, 0.793 mmol) in 1,4-dioxane (5 mL), were added bis(pinacolato)diboron (0.322 g, 1.269 mmol) and potassium acetate (0.234 g, 2.380 mmol) at room temperature. A stream of nitrogen gas was bubbled through the mixture for 15 min. after which PdCl2(dppf)·DCM (0.029 g, 0.040 mmol) was added and the reaction mixture heated to 90° C. for 24 h. The reaction mixture was cooled to room temperature, diluted with EtOAc (10 mL), filtered through the Celite pad. The pad was washed with EtOAc (2×10 mL), the filtrate combined and concentrated under reduced pressure. The crude residue was purified by using MPLC (manually packed SiO₂ cartridge, 230-400 mesh, 18% EtOAc in hexanes) to afford 5 as an off-white semi solid.

Yield: 200 mg (80%)

### Step 5:

To a stirred solution of 6 (140 mg, 0.418 mmol) in 1,4-dioxane (5 mL) and water (1.5 mL), were added 5 (162 mg, 0.543 mmol) and potassium carbonate (173 mg, 1.253 mmol) at 25° C. A stream of nitrogen gas was bubbled through the mixture for 10 min following which PdCl2(dtpbf) (13.61 mg, 0.021 mmol) was added at 25° C., and the reaction mixture heated at 80° C. for 16 h. The reaction mixture was cooled to 25° C., diluted with 10% MeOH in DCM (10 mL), washed with water (3 mL), brine solution (2 mL), dried over Na₂SO₄, filtered, and concentrated under reduced pressure. The crude residue thus obtained was purified by reverse phase preparative HPLC (column: SHIMPACK GIST C18 (10×150 nm) 5 μm; eluents: 10 mM ammonium bicarbonate in water and acetonitrile) to afford Compound 129 as a pale-yellow solid.

Yield: 35 mg (19%)

LCMS: Calculated for C₂₆H₂₅N₃O₃ is 427.50, observed: 428.2 [M+1]⁺

1H-NMR (400 MHz, DMSO-d₆): δ 7.66-7.63 (m, 4H), 7.52 (d, J=8.4 Hz, 2H), 7.35 (d, J=1.2 Hz, 1H), 6.95 (d, J=8.8 Hz, 2H), 6.84 (d, J=0.8 Hz, 1H), 5.69 (t, J=6.0 Hz, 1H), 5.54 (br s, 1H, exchanges with D₂O), 5.37 (d, J=5.6 Hz, 1H, exchanges with D₂O), 4.95-4.92 (m, 1H), 4.78-4.74 (m, 1H), 3.87 (br s, 2H), 3.14-3.08 (m, 1H), 2.98-2.91 (m, 2H), 2.40-2.33 (m, 2H), 1.51 (d, J=6.4 Hz, 3H).

SFC: 96.6%; tR=3.56 min (column: I Cellulose-Z; eluents: CO₂ and 0.5% isopropyl amine in MeOH)

cis-geometry in the tail; diastereomeric ratio=96.6:1.2

### Example A77: Synthesis of Compound 130

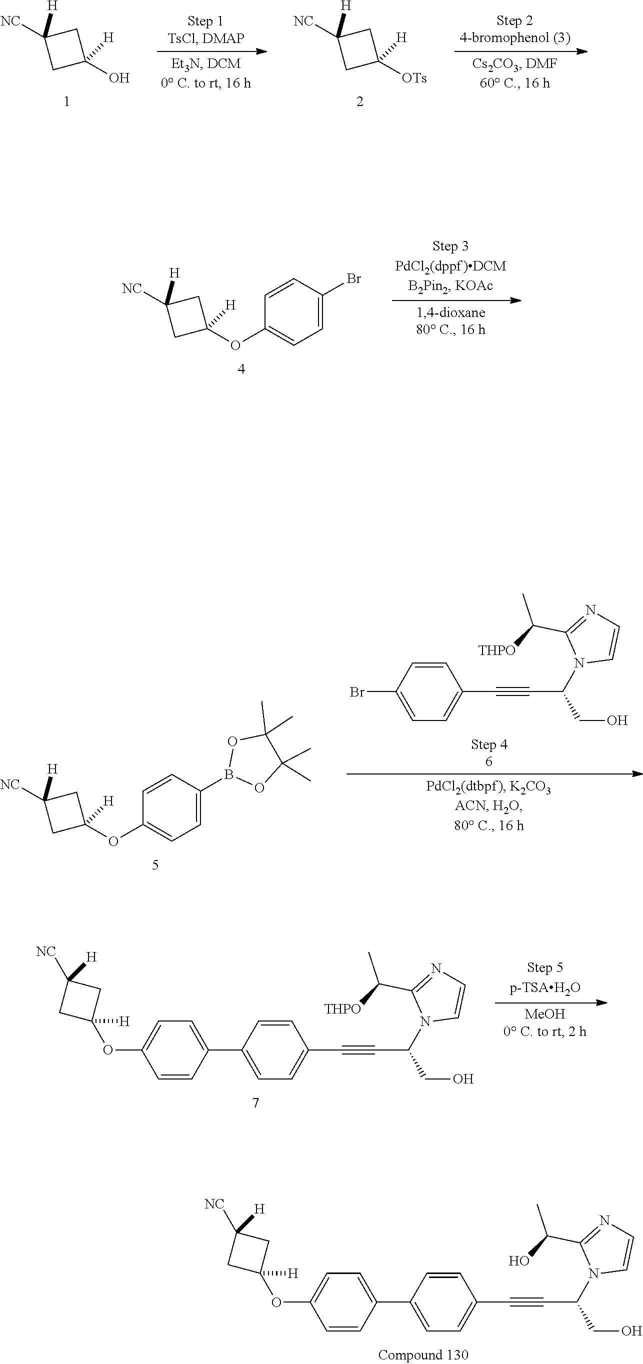

### Step 1:

To a stirred solution of cis-3-hydroxycyclobutane-1-carbonitrile (1, 1 g, 10.30 mmol) in DCM (20 mL), were added Et₃N (4.31 mL, 30.9 mmol), 4-dimethylaminopyridine (0.252 g, 2.059 mmol) and tosyl chloride (2.94 g, 15.45 mmol) at 0° C., and the reaction mixture stirred for 16 h at room temperature. The reaction mixture was quenched with ice water (10 mL) at 0° C. and extracted with DCM (30 mL×2). The combined organic extract was washed with brine solution (20 mL), dried over anhydrous Na₂SO₄, filtered and concentrated under reduced pressure. The crude residue thus obtained was purified by using MPLC (manually packed SiO2 cartridge; 100-200 mesh size; 15% EtOAc in hexanes) to afford 2 as an off-white solid.

Yield: 1.012 g (38%)

### Step 2:

To the stirred solution of 4-bromophenol (3, 723 mg, 4.18 mmol) in DMF (15 mL), were added Cs2CO3 (1.815 g, 5.57 mmol), and 2 (700 mg, 2.79 mmol) at room temperature, and the resulting reaction mixture heated at 60° C. for 16 h. The reaction mixture was quenched with ice-cold water (15 mL) and extracted with EtOAc (2×30 mL). The combined organic layer was washed with brine solution (2×10 mL), dried over anhydrous Na₂SO₄, filtered, and concentrated under reduced pressure. The crude residue thus obtained was purified by using MPLC (manually packed SiO2 cartridge, 230-400 mesh size; 6% EtOAc in hexanes) to afford 4 as an off-white solid.

Yield: 520 mg (71%)

LC-MS: Calculated for C₁₁H₁₀BrNO is 250.99 (exact mass), observed: 251.0 [M]⁺ and 253.1 [M+2]⁺

The identity of trans-geometry of 4 was established through a lack of the nOe enhancement in the methine (3.44 ppm) α- to nitrile upon irradiation of methine α- to phenoxy (4.96 ppm).

### Step 3:

To a stirred solution of 4 (520 mg, 2.063 mmol) in 1,4-dioxane (5 mL), were added bis(pinacolato)diboron (1.048 g, 4.13 mmol) and potassium acetate (607 mg, 6.19 mmol) at room temperature, and a stream of nitrogen gas bubbled through the mixture for 5 min. Pd(dppf)Cl2·DCM (84 mg, 0.103 mmol) was added and the reaction mixture heated at 80° C. for 16 h. The reaction mixture was cooled to room temperature and filtered through a Celite bed. The bed was washed with EtOAc (30 mL×2), the filtrate combined and concentrated under reduced pressure. The crude residue, thus obtained, was purified by using MPLC (manually packed SiO₂ cartridge, 230-400 mesh size; 7% EtOAc in hexanes) to afford boronate 5 as an off-white solid.

Yield: 520 mg (76%)

### Step 4:

To the stirred solution of boronate 5 (428 mg, 1.431 mmol) in acetonitrile (6 mL) and water (1 mL), were added 6 (500 mg, 1.192 mmol), K₂CO₃ (330 mg, 2.385 mmol) at room temperature. A stream of nitrogen gas was bubbled through the mixture for 5 min, following which PdCl₂(dtpbf) (78 mg, 0.119 mmol) was added and the reaction mixture heated to 80° C. and stirred for 16 h. The reaction mixture was cooled to room temperature, diluted with water (10 mL) and extracted with 10% MeOH in DCM (25 mL×3). The combined organic extract was washed with brine solution (20 mL), dried over anhydrous sodium sulphate, filtered, and concentrated under reduced pressure. The crude residue, thus obtained, was purified by using MPLC (manually packed SiO₂ cartridge, 230-400 mesh size; 4% MeOH in DCM) to afford 7 as a brown solid (89% by LCMS; taken to the next step as such without further purification).

Yield: 170 mg (25%)

LC-MS: Calculated for C₃₁H₃₃N₃O₄ is 511.62, observed: 512.3 [M+1]⁺

### Step 4:

To a stirred solution of 7 (170 mg, 0.332 mmol) in MeOH (50 mL), was added p-TSA·H₂O (190 mg, 0.997 mmol) at 0° C., and the resulting reaction mixture stirred at room temperature for 2 h. The reaction mixture was quenched with saturated NaHCO₃ solution (15 mL) and extracted with DCM (3×30 mL). The combined organic layer was washed with brine solution (10 mL), dried over anhydrous Na₂SO₄, filtered, and concentrated under reduced pressure. The crude residue, thus obtained, was purified by using reverse phase preparative HPLC (column: shim pack C18 (150*20 mm), eluents: 10 mM ammonium bicarbonate in water and acetonitrile) to afford Compound 130 as an off-white solid.

Yield: 32 mg (22%)

LC-MS: Calculated for C26H25N3O3 is 427.50, observed: 428.2 [M+1]⁺

1H-NMR (400 MHz, DMSO-d₆): δ 7.66-7.62 (m, 4H), 7.52 (d, J=8.4 Hz, 2H), 7.35 (d, J=1.2 Hz, 1H), 6.96 (d, J=8.4 Hz, 2H), 6.84 (d, J=0.8 Hz, 1H), 5.69 (t, J=5.6 Hz, 1H), 5.53 (t, J=6.0 Hz, 1H, exchanges with D₂O), 5.37 (d, J=5.6 Hz, 1H, exchanges with D₂O), 5.07-5.01 (m, 1H), 4.97-4.91 (m, 1H), 3.87 (t, J=5.6 Hz, 2H), 3.48-3.44 (m, 1H), 2.87-2.80 (m, 2H), 2.55-2.51 (m, 2H, merges with solvent peak), 1.51 (d, J=6.4 Hz, 3H).

SFC: 97.4%; tR=4.00 min (column: I Cellulose-Z; eluents: CO2 and 0.5% isopropyl amine in MeOH). trans-geometry at tail part; diastereomeric ratio=97.45:1.9

### Example A78: Synthesis of Compound 131

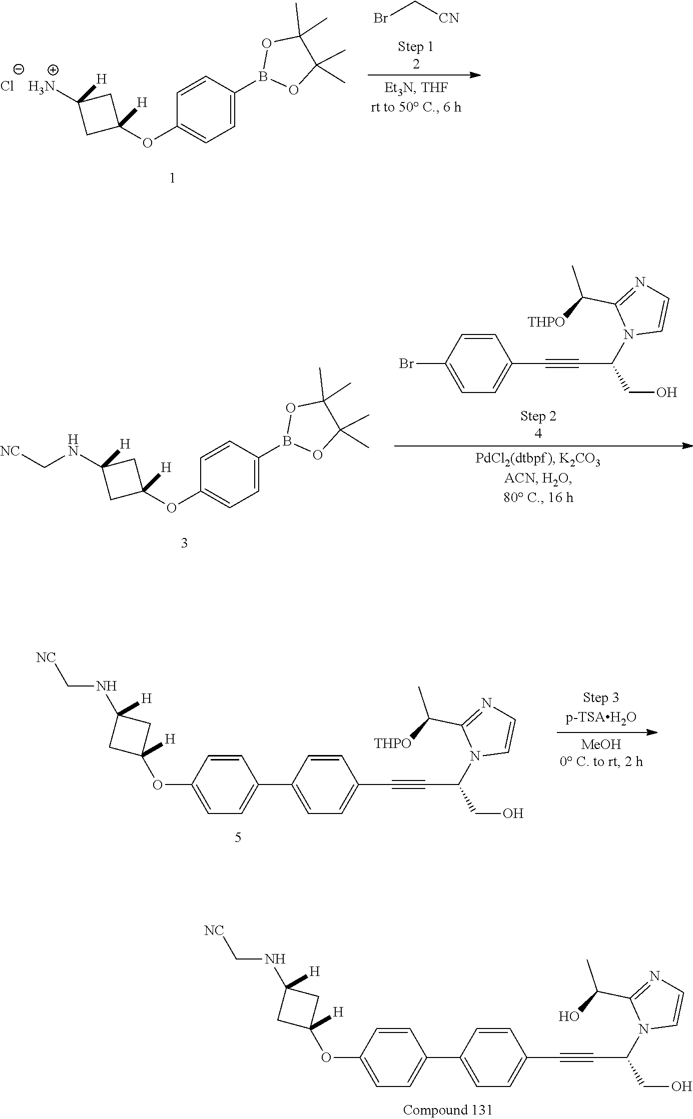

### Step 1:

To stirred solution of 1 (3 g, 9.21 mmol) in THF (5 mL), were added TEA (4.37 mL, 31.1 mmol) and bromoacetonitrile (2, 1.447 mL, 20.75 mmol) at room temperature. The reaction mixture was stirred at 50° C. for 6 h. The reaction was quenched with water (25 mL) and extracted with EtOAc (20 mL×2). The combined organic layer was washed with brine (5 mL), dried over anhydrous Na₂SO₄, filtered and concentrated under reduced pressure to obtain 3 as an off-white solid.

Yield: 3 g (84%)

LC-MS: Calculated for C18H25BN2O3 is 328.22; Observed: 329.3 [M+1]⁺

### Step 2:

To a stirred solution of 3 (1.174 g, 3.58 mmol) in water (2 mL) and acetonitrile (10 mL), were added 4 (1 g, 2.385 mmol) and K2CO3 (0.989 g, 7.15 mmol) at room temperature, and the mixture purged with nitrogen for 5 min. To this reaction mixture, PdCl2(dtbpf) (0.078 g, 0.119 mmol) was added and the purging continued for another 2 min. The reaction mixture was stirred at 80° C. for 16 h. The reaction mixture was quenched with water (50 mL) and extracted with 20% MeOH in DCM (50 mL×2). The combined organic layer was washed with brine (50 mL), dried over anhydrous Na₂SO₄, filtered and concentrated under reduced pressure. The resulting crude mass was purified by using MPLC (manually packed SiO₂ cartridge, 230-400 mesh size; 2% MeOH in DCM) to afford 5 as a brown solid (45% pure by LCMS). The product was taken to the next step without further purification.

Yield: 300 mg (crude)

LC-MS: Calculated for C32H36N4O4 is 540.66; Observed: 541.5 [M+1]⁺

### Step 3:

To a stirred solution of 5 (300 mg, 0.555 mmol) in MeOH (6 mL), was added p-toluene sulfonic acid monohydrate (317 mg, 1.665 mmol) at 0° C. and the resulting reaction mixture stirred at room temperature for 2 h. The reaction mixture was concentrated and basified with sat. NaHCO₃ solution (10 mL). The aqueous layer was extracted with 20% MeOH in DCM (10 mL×2). The combined organic extract was dried over anhydrous Na₂SO₄, filtered and concentrated under reduced pressure. The crude residue, thus obtained, was purified by reverse phase column chromatography (Column: Redisep, C-18 silica gel; 10 mM ammonium bicarbonate in water and acetonitrile) to afford Compound 131 as a white solid.

Yield: 60 mg (23%)

LC-MS: Calculated for C27H28N4O3 is 456.5; Observed: 457.4 [M+1]⁺

1H-NMR (400 MHz, DMSO-d6): δ 7.66-7.61 (m, 4H), 7.51 (d, J=8.40 Hz, 2H), 7.35 (d, J=1.20 Hz, 1H), 6.95 (d, J=8.80 Hz, 2H), 6.84 (d, J=1.20 Hz, 1H), 5.69 (t, J=6.00 Hz, 1H), 5.53 (t, J=5.60 Hz, 1H, exchanges with D2O), 5.37 (d, J=5.60 Hz, 1H, exchanges with D2O), 4.95-4.92 (m, 1H), 4.50-4.47 (m, 1H), 3.87 (t, J=6.00 Hz, 2H), 3.57 (d, J=6.80 Hz, 2H), 3.01-2.99 (m, 1H), 2.92-2.89 (m, 1H), 2.85-2.81 (m, 2H), 1.87-1.84 (m, 2H), 1.51 (d, J=6.40 Hz, 3H).

SFC: 95.5%; tR=2.95 min (Column: l-cellulose B; Eluents: CO2 and 0.5% isopropyl amine in MeOH)

### Example A79: Synthesis of Compound 132

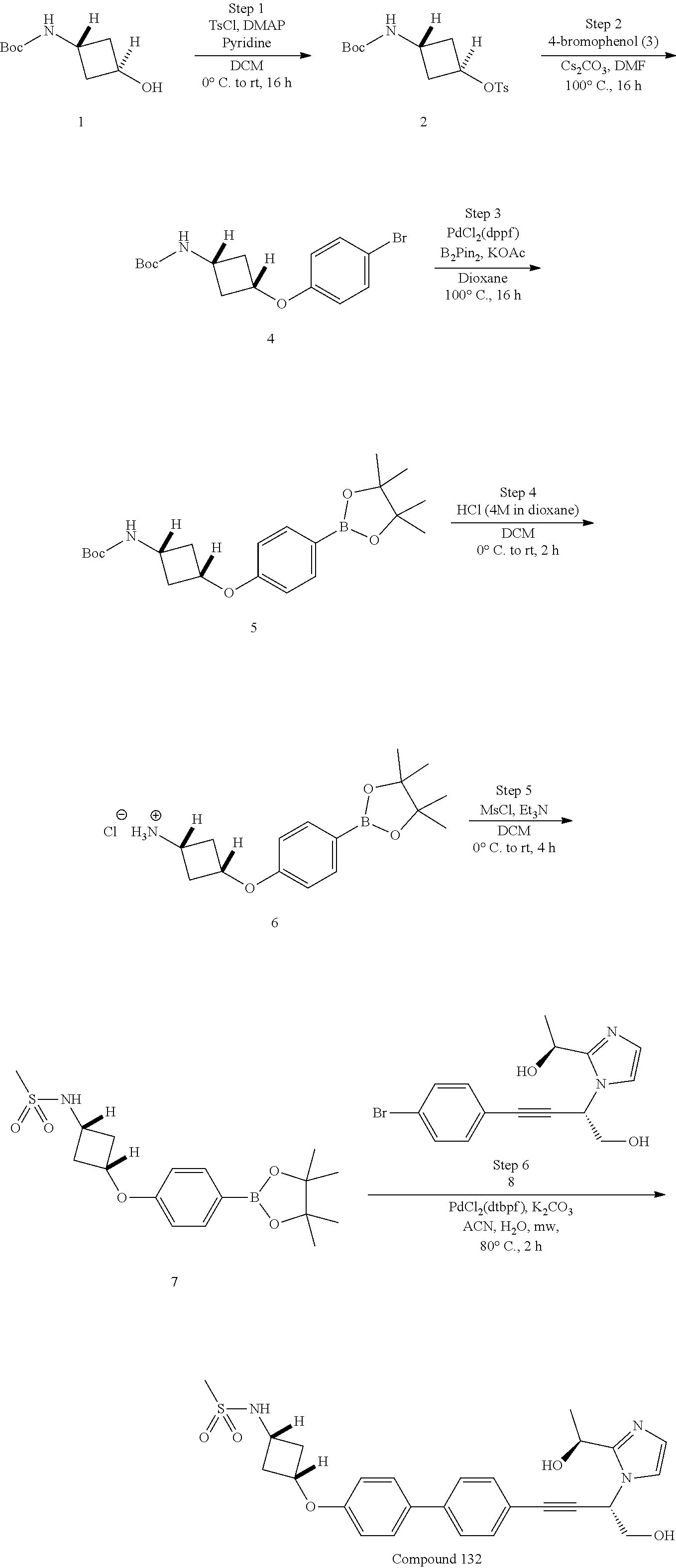

### Step 1:

To a stirred solution of the tert-butyl ((1r,3r)-3-hydroxycyclobutyl)carbamate (1, 10 g, 53.4 mmol) in DCM (100 mL), were added DMAP (6.52 g, 53.4 mmol), pyridine (21.60 mL, 267 mmol) and tosyl chloride (11.20 g, 58.7 mmol) at 0° C., and the reaction mixture stirred at room temperature for 16 h. The reaction mixture was then concentrated under reduced pressure. To the resulting residue, water (250 mL) was added and extracted with MTBE (250 mL×2). The combined organic layer was washed with sat. NaHCO₃ (250 mL×2), brine (30 mL), dried over anhydrous Na₂SO₄, filtered and concentrated under reduced pressure to obtain 2 as a white solid.

Yield: 15 g (81%)

LC-MS: Calculated for C16H23NO5S is 341.42; observed:642.2 [2M+1]⁺

### Step 2:

To stirred solution of 2 (15 g, 43.9 mmol) in DMF (200 mL), were added 4-bromophenol (3, 8.36 g, 48.3 mmol) and cesium carbonate (42.9 g, 132 mmol) at room temperature, and the reaction stirred at 100° C. for 16 h. The reaction mixture was then quenched with water (250 mL) and extracted with DCM (250 mL×2). The combined organic layer was washed with brine (30 mL), dried over anhydrous Na₂SO₄, filtered and concentrated under reduced pressure. The crude residue was purified by MPLC (using manually packed SiO₂ cartridge, 230-400 mesh; 12% EtOAc in hexanes) to afford 4 as a white solid. LCMS showed 75% purity; the product was taken to the next step without further purification.

Yield: 3.8 g (19%)

LC-MS: Calculated for C15H20BrNO3 is 342.2; observed: 242.2 [M-Boc]⁺ and 244.0 [(M-Boc)+2]⁺

The stereochemistry was of 4 was confirmed by nOe studies.

### Step 3:

To a stirred solution of 4 (2.8 g, 8.18 mmol) in 1,4-dioxane (50 mL), were added bis(pinacolato)diboron (3.12 g, 12.27 mmol) and potassium acetate (2.409 g, 24.54 mmol) at room temperature and the resulting reaction mixture purged with nitrogen for 5 min. To this reaction mixture, PdCl2(dppf) (0.299 g, 0.409 mmol) was added and the purging continued for another 2 min. The reaction mixture was then stirred at 100° C. for 16 h. The reaction mixture was quenched with water (100 mL) and extracted with EtOAc (50 mL×2). The combined organic layer was washed with brine (5 mL), dried over anhydrous Na₂SO₄, filtered and concentrated under reduced pressure. The crude mass, thus obtained, was purified by using MPLC (using manually packed SiO₂ cartridge, 230-400 mesh size; 15% EtOAc in hexanes) to afford 5 as a pale-yellow solid. LCMS showed 77% purity; the product was taken as such to the next step without further purification.

Yield: 1.2 g (29%)

LC-MS: Calculated for C21H32BNO5 is 389.2; Observed: 290.2 [(M-Boc)+1]⁺

### Step 4:

To a stirred solution of 5 (1.2 g, 3.08 mmol) in anhydrous DCM (10 mL), was added HCl (4.0 M in 1,4 dioxane, 7.71 ml, 30.8 mmol) at 0° C. and the resulting mixture stirred at ambient temperature for 2 h. The volatiles were removed from the reaction mixture under reduced pressure. The crude residue was triturated with n-pentane (10 mL), decanted, and dried under reduced pressure to afford 6 as an off-white solid.

Yield: 800 mg (80%)

LC-MS: Calculated for C16H25BNO3⁺ is 290.19; Observed: 290.3 [M]⁺

### Step 5:

To a stirred solution of 6 (0.3 g, 0.921 mmol) in DCM (6 mL), were added TEA (0.385 mL, 2.76 mmol) and methane sulfonyl chloride (0.108 mL, 1.382 mmol) at 0° C., and the reaction stirred at room temperature for 4 h. The reaction mixture was then concentrated, quenched with water (50 mL) and extracted with MTBE (20 mL×2). The combined organic layer was washed with brine (5 mL), dried over anhydrous Na₂SO₄, filtered and concentrated under reduced pressure. The resulting crude residue was purified by MPLC (using manually packed SiO₂ cartridge, 230-400 mesh; 10% EtOAc in hexanes) to afford 7 as a white solid

Yield: 0.3 g (72%)

### Step 6:

To a stirred solution of 7 (316 mg, 0.859 mmol) in acetonitrile (4 mL) and water (4 mL), were added 8 (240 mg, 0.716 mmol) and K₂CO₃ (297 mg, 2.148 mmol) at room temperature. The resulting mixture was purged with nitrogen for 5 min. To this reaction mixture, PdCl2(dtbpf) (23.33 mg, 0.036 mmol) was added and the purging continued for another 2 min. The resulting reaction mixture was irradiated in microwave reactor at 80° C. for 2 h. The reaction mixture was quenched with water (10 mL) and extracted with 10% MeOH in DCM (10 mL×2). The combined organic layer was washed with brine (5 mL), dried over anhydrous Na₂SO₄, filtered and concentrated to afford a crude residue. The crude residue was purified by using reverse phase column chromatography (Column: Redisep, C18 silica; 10 mM ammonium bicarbonate in water and acetonitrile) to afford 100 mg as an off-white solid. ¹H NMR indicated extraneous peaks and the purity by SFC was 75%. The resulting 100 mg was purified by using SFC (Column: I CELLULOSE Z-(250×30) mm, 5 μm; Eluents: CO2:0.5% isopropyl amine in MeOH [60:40]) to get 50 mg as an off-white solid. ¹H NMR indicated extraneous peaks; the compound was further repurified by using reverse phase column chromatography (Column: Redisep, C18 column; 10 mM ammonium bicarbonate in water and acetonitrile) to afford Compound 132 as an off-white solid.

Yield: 30 mg (39%)

LC-MS: Calculated for C26H29N3O5S is 495.5; observed: 495.8 [M+1]⁺

1H-NMR (400 MHz, DMSO-d6): δ 7.66-7.62 (m, 4H), 7.51 (d, J=8.40 Hz, 2H), 7.46 (d, J=8.80 Hz, 1H, 1H, exchanges with D2O), 7.35 (d, J=1.20 Hz, 1H), 6.94 (d, J=8.80 Hz, 2H), 6.84 (d, J=1.20 Hz, 1H), 5.69 (t, J=6.00 Hz, 1H), 5.53 (t, J=5.60 Hz, 1H, exchanges with D2O), 5.37 (d, J=5.60 Hz, 1H, exchanges with D2O), 4.95-4.92 (m, 1H), 4.49-4.45 (m, 1H), 3.87 (t, J=6.00 Hz, 2H), 3.54-3.52 (m, 1H), 2.95-2.90 (m, 2H), 2.88 (s, 3H), 2.33-2.05 (m, 2H), 1.51 (d, J=6.80 Hz, 3H). cis-geometry at cyclobutyl ring; SFC purity=99.3% (Cellulose-Z_0.5% IPAm in MeOH_40 tR=3.79 min)

### Example A80: Synthesis of Compound 133

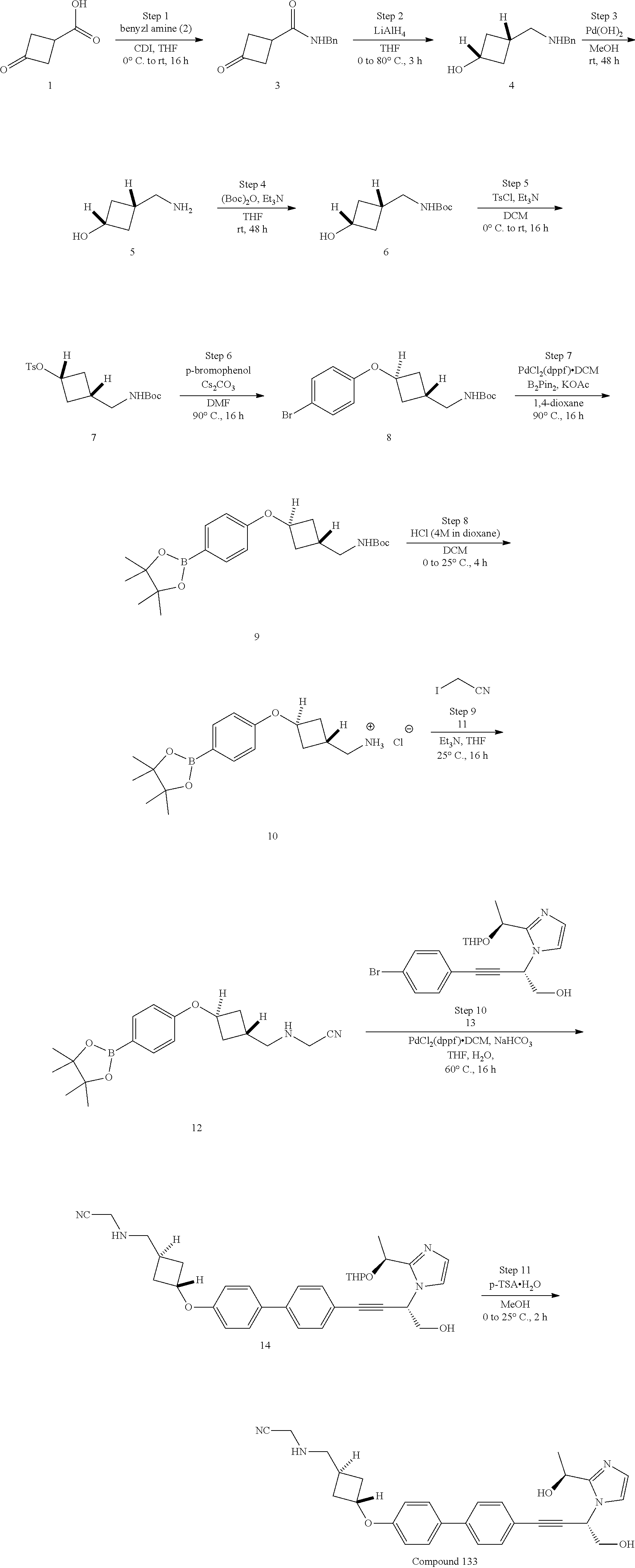

### Step 1:

To a stirred solution of 3-oxocyclobutane-1-carboxylic acid (1, 1.0 g, 8.76 mmol) in THF (10 mL), was added carbonyl diimidazole (1.853 g, 13.15 mmol) at 0° C. and the reaction mixture stirred at room temperature for 1 h. Subsequently, benzylamine (2, 1.409 g, 13.15 mmol) was added and the reaction mixture stirred for 16 h at 25° C. The reaction mixture was concentrated and the crude residue, thus obtained, purified by using MPLC (manually packed SiO₂ cartridge, 230-400 mesh; 95% EtOAc in hexanes) to afford 3 as a white solid.

Yield: 1 g (52%)

LC-MS: Calculated for C₁₂H₁₃NO₂ is 203.24, Observed: 204.2 [M+1]⁺

### Step-2:

To a stirred solution of 3 (1.0 g, 4.92 mmol) in THF (10 mL), was added lithium aluminum hydride (2 M in THF) (4.92 mL, 9.84 mmol) at 0° C. The reaction mixture was stirred at 80° C. for 3 h. The reaction was then cooled to 0° C., quenched with saturated Na₂SO₄ solution (8 mL) and EtOAc (10 mL) and the resultant mass stirred for 1 h at 25° C. The mixture was filtered through a pad of Celite pad and washed with THF (20 mL) and the filtrate dried over anhydrous Na₂SO₄, filtered and concentrated under reduced pressure to afford 4 as a pale-brown liquid.

Yield: 1.0 g (89%)

LC-MS: Calculated for C₁₂H₁₇NO is 191.27, Observed: 192.2 [M+1]⁺

### Step-3:

To a stirred solution of 4 (2.5 g, 13.07 mmol) in MeOH (25 mL), was added 10-20% palladium hydroxide on carbon (2.0 g). and the resulting reaction mixture stirred for 48 h at 25° C. under a blanket of hydrogen. The reaction mixture was filtered through a pad of Celite and washed with MeOH (30 mL). The resulting filtrate was concentrated under reduced pressure to afford 5 as a white solid. The crude product was taken to the next step without any further purification.

Yield: 1.1 g (63%)

LC-MS: Calculated for C₅H₁₁NO is 101.15, Observed: 102.2 [M+1]⁺.

Another batch was carried out on 5 g of 4 to get 3.0 g of 5

### Step-4:

To a stirred solution of 5 (2.5 g, 24.72 mmol) in THE (15 mL), were added Et3N (10.34 mL, 74.1 mmol) and di-tert-butyl dicarbonate (6.47 g, 29.7 mmol) at 0° C. and the reaction mixture stirred at room temperature for 16 h. The reaction mixture was concentrated and the crude residue, thus obtained, was purified by using MPLC (manually packed SiO₂ cartridge, 230-400 mesh; 5% MeOH in DCM) to afford 6 as a white solid.

Yield: 4.0 g (69%)

LC-MS: Calculated for C₁₀H₁₉NO₃ is 201.27, Observed: 102.3 [(M-Boc)+1]⁺.

### Step-5:

To a stirred solution of 6 (4 g, 19.87 mmol) in DCM (25 mL), were added Et3N (8.38 mL, 59.6 mmol) and tosyl chloride (5.68 g, 29.8 mmol) at 0° C. The reaction mixture was stirred at 25° C. for 16 h. The reaction was quenched with water (25 mL) and extracted with DCM (25 mL×2). The combined organic layer was washed with 10% NaHCO₃ (2×10 mL), brine (25 mL), dried over anhydrous Na₂SO₄, filtered and concentrated under reduced pressure. The crude residue, thus obtained, was purified by using MPLC (manually packed SiO₂ cartridge, 230-400 mesh size; 355% EtOAc in hexanes) to afford 7 as a white solid.

Yield: 4.6 g (65%)

LC-MS: Calculated for C₁₇H₂₅NO₅S is 355.45 Observed: 354.2 [M−1]⁻

### Step-6:

To a stirred solution of 7 (4.44 g, 12.48 mmol) in DMF (20 mL), were added cesium carbonate (6.78 g, 20.81 mmol) and 4-bromophenol (1.8 g, 10.40 mmol) at room temperature. The reaction mixture was stirred at 90° C. for 16 h. The reaction was quenched with cold water (25 mL) and extracted with EtOAc (25 mL×2). The combined organic layer was washed with brine (30 mL), dried over anhydrous Na₂SO₄, filtered and concentrated under reduced pressure. The crude residue, thus obtained, was purified by using MPLC (manually packed SiO₂ cartridge, 230-400 mesh; 20% EtOAc in hexanes) to afford 8 as a white solid.

Yield: 3.5 g (93%)

LC MS: Calculated for C₁₆H₂₂BrNO₃ is 356.26, Observed: 356 [M] and 354.2 [M−2]⁻

The stereochemistry was confirmed by nOe studies.

### Step-7:

To a stirred solution of 8 (4 g, 11.23 mmol) in 1,4-dioxane (50 mL), were added bis(pinacolato)diboron (3.71 g, 14.60 mmol) and potassium acetate (3.31 g, 33.7 mmol) at room temperature. The reaction mixture was purged with nitrogen for 5 min. To this reaction mixture, PdCl₂(dppf)·CH₂Cl₂ adduct (0.917 g, 1.123 mmol) was added and the purging continued for 10 min and then heated at 90° C. for 16 h. The reaction mixture was filtered through a pad of Celite and washed with EtOAc (30 mL). The combined filtrate was dried over anhydrous Na₂SO₄, filtered and concentrated under reduced pressure. The resulting crude residue was purified by using MPLC (manually packed SiO₂ cartridge, 230-400 mesh; 30% EtOAc in hexanes) to afford 9 as a pale-yellow solid.

Yield: 4.0 g (85%)

LC-MS: Calculated for C₂₂H₃₄BNO₅ is 403.33 Observed: 404.4 [M+1]⁺

### Step-8:

To a stirred solution of 9 (2.0 g, 4.96 mmol) in anhydrous DCM (10 mL), was added hydrochloric acid (4 M in dioxane, 2.5 mL, 9.92 mmol) at 0° C. and the resulting mixture stirred at 25° C. for 4 h. The reaction mixture was concentrated under reduced pressure. The crude was triturated with hexane (20 mL) to obtain 10 as an off-white solid.

Yield: 1.3 g (76%)

LC-MS: Calculated for C17H27BNO3⁺ is 304.22, Observed: 304.2 [M]⁺

### Step-9:

To a stirred solution of 10 (1.3 g, 3.83 mmol) in anhydrous THE (15 mL), were added Et3N (1.596 mL, 11.48 mmol) and Iodoacetonitrile (11, 0.320 mL, 4.59 mmol) at 25° C. and the reaction mixture stirred for 16 h. The reaction was quenched with water (15 mL) and extracted with EtOAc (25 mL×2). The combined organic layer was washed with brine (15 mL), dried over anhydrous Na₂SO₄, filtered and concentrated under reduced pressure. The crude residue thus obtained was purified by using MPLC (manually packed SiO₂ cartridge, 230-400 mesh; 92% EtOAc in hexanes) to afford 12 as a colourless gummy liquid.

Yield: 1.0 g (75%)

LC-MS: Calculated for C₁₉H₂₇BN₂O₃ is 342.25, Observed: 343.3 [M+1]⁺.

### Step-10:

To a stirred solution of 12 (392 mg, 1.145 mmol) in THF (4 mL) and water (1 mL), were added 13 (400 mg, 0.954 mmol) and NaHCO₃ (160 mg, 1.908 mmol) at room temperature. The reaction mixture was purged for 10 min. To this reaction mixture, PdCl₂(dppf)·CH₂Cl₂ adduct (78 mg, 0.095 mmol) was added and the purging continued for 10 min. The reaction mixture was stirred at 60° C. for 16 h. The reaction mixture was filtered through a pad of Celite bed and washed with EtOAc (20 mL). The combined filtrate was dried over anhydrous Na₂SO₄, filtered and concentrated under reduced pressure. The crude residue, thus obtained, was purified by using MPLC (manually packed SiO₂ cartridge, 230-400 mesh; 8% MeOH in DCM) to afford 14 as a brown solid.

Yield: 260 mg (32%)

LC-MS: Calculated for C₃₃H₃₈N₄O₄ is 554.69, Observed: 555.5 [M+1]⁺.

### Step-11:

To a stirred solution of 14 (260 mg, 0.469 mmol) in MeOH (5 mL), was added p-toluenesulfonic acid monohydrate (178 mg, 0.937 mmol) at 0° C. The resulting reaction mixture was stirred at 25° C. for 2 h. The reaction mixture was concentrated and basified with sat. NaHCO₃ solution (25 mL). The aqueous layer was extracted with 10% MeOH in DCM (20 mL×3). The combined organic extract was dried over anhydrous Na₂SO₄, filtered and concentrated under reduced pressure. The resulting crude residue was purified by MPLC (Column: RediSep Gold, C18 reversed phase SiO2, 100 gram; Eluents:10 mM ammonium bicarbonate in water and ACN) to obtain Compound 133 as a white solid.

Yield: 35 mg (15%)

LC-MS: Calculated for C₂₈H₃₀N₄O₃ is 470.57 Observed: 471.2 [M+1]⁺

1H-NMR (400 MHz, DMSO-d6): δ 7.66-7.61 (m, 4H), 7.51 (d, J=8.40 Hz, 2H), 7.36 (app d, J=0.80 Hz, 1H), 6.91 (d, J=8.80 Hz, 2H), 6.84 (app d, J=0.80 Hz, 1H), 5.69 (t, J=5.60 Hz, 1H), 5.53 (t, J=5.60 Hz, 1H, exchanges with D2O), 5.37 (d, J=5.60 Hz, 1H, exchanges with D2O), 4.96-4.92 (m, 1H), 4.86-4.83 (m, 1H), 3.87 (t, J=5.60 Hz, 2H), 3.64 (s, 2H), 2.70 (br s, 2H), 2.55-2.51 (m, 1H, exchanges with D2O, merges with solvent peak), 2.40 (br s, 1H), 2.31-2.25 (m, 2H), 2.18-2.13 (m, 2H), 1.51 (d, J=6.40 Hz, 3H).

SFC: 97.2%; Column: 1 Cellulose-Z; Eluents: 0.5% IPAm in MeOH and CO2; tR=2.84 min. trans-geometry in the tail part; SFC purity=97.2%

### Example A81: Synthesis of Compound 134

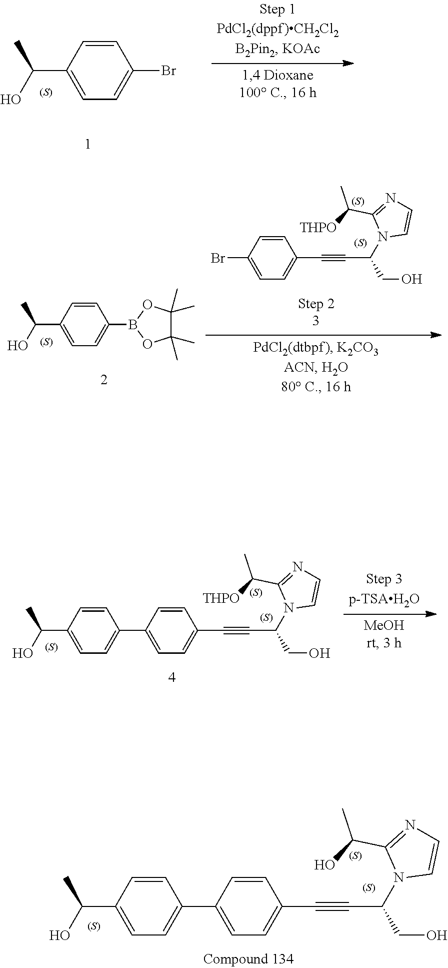

### Step-1:

To a solution of (S)-1-(4-bromophenyl)ethan-1-ol (1, 2 g, 9.95 mmol) in 1,4-dioxane (30 mL), were added potassium acetate (2.93 g, 29.8 mmol) and bis(pinacolato)diboron (3.79 g, 14.92 mmol) at room temperature. The resulting mixture was purged with nitrogen for 10 min. To this reaction mixture, PdCl₂(dppf)·CH₂Cl₂ adduct (0.812 g, 0.995 mmol) was added and purging with nitrogen was continued for another 2 min. The resulting reaction mixture was stirred at 100° C. for 16 h. The reaction mixture was cooled to room temperature, inorganic solids were filtered through Celite pad and washed with EtOAc (100 mL). The combined filtrate was washed with brine solution (50 mL), dried over anhydrous Na₂SO₄, filtered and concentrated under reduced pressure. The resulting crude mass was purified by MPLC (using manually packed SiO₂ cartridge 100-200 mesh size; 10% EtOAc in hexanes) to afford 2 as a pale-yellow gum. Yield: 2 g (73%)

### Step-2:

To a stirred solution of 3 (800 mg, 1.908 mmol) in acetonitrile (10 mL) and water (10 mL), were added 2 (710 mg, 2.86 mmol) and K₂CO₃ (791 g, 5.72 mmol) at room temperature. The reaction mixture was purged with nitrogen for 5 min. To this reaction mixture, PdCl₂(dtbpf) (124 mg, 0.191 mmol) was added and purging with nitrogen was continued for another 2 min. The resulting reaction mixture was stirred at 80° C. for 16 h. The reaction mixture was quenched with water (50 mL), extracted with 10% MeOH in DCM (50 mL×2). The combined organic layer was washed with brine solution (25 mL), dried over anhydrous Na₂SO₄, filtered and concentrated under reduced pressure. The resulting crude product was purified by MPLC (using manually packed SiO₂ cartridge, 230-400 mesh size; 5% MeOH in DCM) to afford 4 as brown solid.

Yield: 450 mg (49%)

LC-MS: Calculated for C₂₈H₃₂N₂O₄ is 460.57, Observed: 461.3 [M+1]⁺

### Step-3:

To a stirred solution of 4 (450 mg, 0.977 mmol) in MeOH (20 mL), was added p-toluenesulfonic acid monohydrate (558 mg, 2.93 mmol) at 0° C. and the reaction mixture was warmed to room temperature and stirred for 3 h. The volatiles were evaporated under reduced pressure and the resulting residue basified with 10% NaHCO₃ solution (30 mL) and extracted with 10% MeOH in DCM (50 mL×2). The combined organic layer was washed with brine (30 mL), dried over anhydrous Na₂SO₄, filtered and concentrated under reduced pressure. The product was purified by reverse phase preparative HPLC (Column: Shimpack C18 (150*20 mm) 5 μm; Eluents: 10 mM ammonium bicarbonate in water and ACN) to get 240 mg of product. 1H NMR showed extraneous peaks with SFC purity 94.7%. The product was re-purified by SFC (Column: LUX-I-A3 (250*20) mm, 5 μm; Eluents: CO₂:0.5% isopropylamine in MeOH [65:35]). The fractions were concentrated and resulting residue dissolved in DCM (50 mL) and washed with water (20 mL). The organic layer was dried over anhydrous Na2SO4, filtered, concentrated and lyophilized to afford Compound 134 as a white solid.

Yield=150 mg (41%)

LC-MS: Calculated for C₂₃H₂₄N₂O₃ is 376.45, Observed: 377.3 [M+1]⁺

1H-NMR (400 MHz, DMSO-d6): δ 7.69 (d, J=8.40 Hz, 2H), 7.64 (d, J=8.00 Hz, 2H), 7.54 (d, J=8.40 Hz, 2H), 7.44 (d, J=8.00 Hz, 2H), 7.36 (d, J=1.20 Hz, 1H), 6.84 (d, J=1.20 Hz, 1H), 5.69 (t, J=6.00 Hz, 1H), 5.57 (br s, 1H, exchanges with D2O), 5.40 (d, J=5.20 Hz, 1H, exchanges with D2O), 5.20 (d, J=4.00 Hz, 1H, exchanges with D2O), 4.97-4.91 (m, 1H), 4.80-4.74 (m, 1H), 3.88-3.87 (m, 2H), 1.51 (d, J=6.40 Hz, 3H), 1.35 (d, J=6.40 Hz, 3H). Single isomer with SFC purity=99.4% (LUX-I-Amylose3_0.5% IPAm in MeOH tR=2.48 min.)

### Example A82: Synthesis of Compound 135

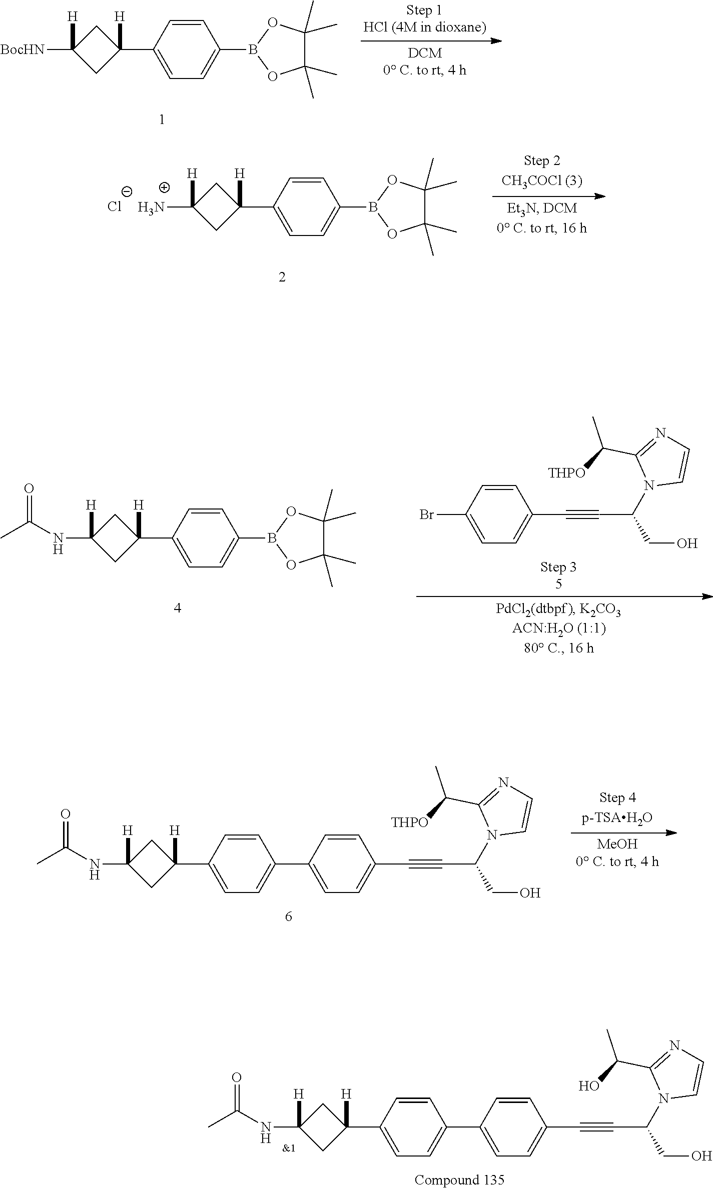

### Step-1:

To a stirred solution of the 1 (3 g, 8.04 mmol) in DCM (40 mL), was added HCl (4 M in dioxane, 10.0 mL, 40.2 mmol) at 0° C. and the resulting mixture stirred at room temperature for 4 h. The volatiles were removed under reduced pressure. The crude residue was co-distilled with toluene (30 mL×2). The solid obtained was triturated with 50% EtOAc in hexanes (100 mL). The solid was filtered and dried in vacuo to afford 2 as an off-white solid.

Yield=1.9 g (crude product)

LCMS: Calculated for C16H25BNO2⁺ is 274.19; Observed: 274.4 [M]⁺

### Step-2:

To a solution of 2 (1.9 g, 6.14 mmol) in DCM (10 mL), were added triethylamine (2.57 mL, 18.41 mmol) and acetyl chloride (3, 0.53 mL, 7.36 mmol) at 0° C. and the resulting reaction mixture stirred at room temperature for 16 h. The reaction was quenched with 10% NaHCO₃ solution (20 mL) and extracted with DCM (30 mL×2). The combined organic layer was washed with brine (20 mL) and dried over anhydrous Na₂SO₄, filtered and concentrated under reduced pressure. The resulting crude residue was purified by MPLC (manually packed SiO₂ cartridge, 230-400 mesh size; 80% EtOAc in hexanes) to afford 4 as an off-white solid.

Yield=1.3 g (56%)

LCMS: Calculated for C18H26BNO3 is 315.22; Observed: 316.3 [M+1]⁺

### Step-3:

To a solution of 5 (0.5 g, 1.19 mmol) in acetonitrile (5 mL) and water (5 mL), were added 4 (0.56 g, 1.79 mmol) and K₂CO₃ (0.49 g, 3.58 mmol) at room temperature, and the resulting mixture purged with nitrogen for 5 min. To this reaction mixture, PdCl₂(dtbpf) (0.078 g, 0.12 mmol) was added and stirred at 80° C. for 16 h. The reaction mixture was quenched with water (20 mL) and extracted with 10% MeOH in DCM (20 mL×2). The combined organic layer was washed with brine (10 mL) and dried over anhydrous Na₂SO₄, filtered and concentrated under reduced pressure. The resulting crude residue was purified by MPLC (using manually packed SiO₂ cartridge, 230-400 mesh size; 5% MeOH in DCM) to obtain 6 as pale-brown solid

Yield: 300 mg (47%)

LCMS: Calculated for C32H37N3O4 is 527.66; Observed: 528.3 [M+1]⁺

### Step-4:

To a stirred solution of 6 (0.3 g, 0.57 mmol) in MeOH (10 mL), was added p-toluenesulfonic acid monohydrate (0.32 g, 1.70 mmol) at 0° C. and the reaction mixture stirred at room temperature for 4 h. The volatiles were then removed under reduced pressure. The resulting residue was dissolved in 10% MeOH in DCM (100 mL), washed with 10% NaHCO3 solution (20 mL). The organic layer was dried over anhydrous Na₂SO₄, filtered and concentrated under reduced pressure. The resulting crude residue was purified by reverse phase silica (Column: Redisep C18; Eluents: 10 mM ammonium bicarbonate in water and ACN) to afford the desired product as white solid.

The obtained compound showed ˜5% of trans-isomer, which was separated by SFC (Column: LUXIA3-(250*20) mm, 5 μm; Eluents: CO₂:0.5% isopropyl amine in MeOH and ACN [60:40]) to afford Compound 135 as white solid.

Yield: 80 mg (31%)

LCMS: Calculated for C27H29N3O3 is 443.55; Observed: 444.2 [M+1]⁺

1H-NMR (400 MHz, DMSO-d6): δ 8.14 (d, J=8.00 Hz, 1H), 7.70 (dd, J=2.00, 6.80 Hz, 2H), 7.65 (d, J=8.40 Hz, 2H), 7.54 (dd, J=1.60, 6.60 Hz, 2H), 7.37-7.35 (m, 3H), 6.84 (app d, J=1.20 Hz, 1H), 5.70 (t, J=5.60 Hz, 1H), 5.54 (t, J=5.60 Hz, 1H, exchanges with D2O), 5.38 (d, J=5.60 Hz, 1H, exchanges with D2O), 4.96-4.93 (m, 1H), 4.23-4.21 (m, 1H), 3.87 (t, J=6.00 Hz, 2H), 3.19-3.14 (m, 1H), 2.65-2.59 (m, 2H), 2.04-1.96 (m, 2H), 1.79 (s, 3H), 1.51 (d, J=6.40 Hz, 3H).

SFC purity: 100% (Column: LUX-l-Amylose3; Eluents: 0.5% isopropylamine in ACN and MeOH and CO2); tR=5.76 min. cis-geometry in the tail part; single isomer with 100% SFC purity.

### Example A83: Synthesis of Compound 136

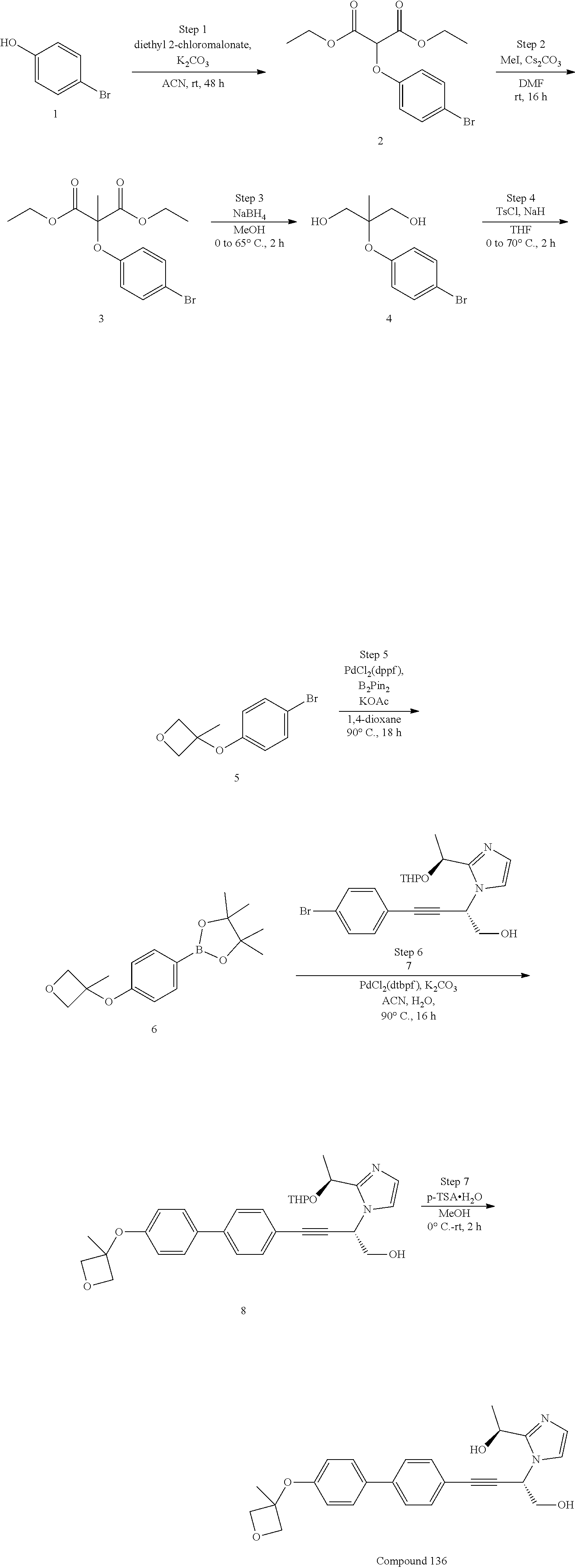

### Step 1:

To a stirred solution of 4-bromophenol (1, 5 g, 28.9 mmol) and diethyl 2-chloromalonate (4.94 mL, 30.3 mmol) in acetonitrile (50 mL), was added K₂CO₃ (9.99 g, 72.3 mmol) at room temperature. The reaction mixture was stirred at room temperature for 48 h. The reaction mixture was quenched with water (200 mL). This was extracted with EtOAc (2×200 mL). The combined organic layer was washed with brine (200 mL), dried over anhydrous Na2SO4, filtered and the filtrate was concentrated under reduced pressure. The crude thus obtained was purified by MPLC (using manually packed SiO2 cartridge, 230-400 mesh size; 5% EtOAc in hexanes) to get 2 as a light-yellow gum.

Yield: 7 g (69%)

### Step 2:

To a stirred solution of 2 (7 g, 21.14 mmol) in anhydrous DMF was added Cs₂CO₃ (10.33 g, 31.7 mmol). The resulting reaction mixture was stirred at room temperature for 30 min. Then iodomethane (1.974 mL, 31.7 mmol) was added and stirred at room temperature for 16 h. The reaction mixture was quenched with water (50 mL) and this was extracted with EtOAc (2×100 mL). The combined organic layer was washed with brine (50 mL), dried over anhydrous Na₂SO₄, filtered and concentrated under reduced pressure to get 3 as a colorless liquid.

Yield: 6.5 g (71%)

### Step 3:

To a stirred solution of 3 (6.5 g, 18.83 mmol) in MeOH (60 mL), was added NaBH₄ (7.12 g, 188 mmol) portion-wise over 40 min at 0° C. The reaction mixture was slowly warmed to room temperature and then heated at 65° C. for 2 h. The reaction mixture was quenched with water (60 mL) and extracted with EtOAc (2×100 mL). The combined organic layer was washed with brine (60 mL), dried over anhydrous Na₂SO₄, filtered and the filtrate was concentrated under reduced pressure. The crude thus obtained was purified by MPLC (using manually packed SiO₂ cartridge, 100-200 mesh; 40% EtOAc in hexanes) to get 4 as a white solid

Yield: 3.5 g (64%)

### Step 4:

To a stirred solution of 4 (2 g, 7.66 mmol) in anhydrous THF (25 mL), was added sodium hydride (0.337 g, 8.43 mmol) portion wise at 0° C. The reaction mixture was stirred at 0° C. for 40 min and then a solution of 4-toluenesulfonyl chloride (1.460 g, 7.66 mmol) in THE (5 mL) was added. The resulting mixture was stirred at 0° C. for 30 min. After this time, sodium hydride (0.337 g, 8.43 mmol) was added portion wise. The reaction mixture was warmed to room temperature and stirred for 20 min. The reaction mixture was then heated to 70° C. for 2 h. The reaction mixture was quenched with water (30 mL) and extracted with EtOAc (2×50 mL). The combined organic layer was washed with brine (50 mL), dried over anhydrous Na₂SO₄, filtered and the filtrate was concentrated under reduced pressure. The crude residue was purified by MPLC (using manually packed SiO₂ cartridge, 100-200 mesh; 10% EtOAc in hexanes) to get 5 as a colorless liquid.

Yield: 1 g (51%)

### Step 5:

To a stirred the solution of 5 (1 g, 4.11 mmol) in 1,4-dioxane (10 mL), were added potassium acetate (1.211 g, 12.34 mmol) and bis(pinacolato)diboron (1.567 g, 6.17 mmol) at a room temperature. The reaction mixture was purged with nitrogen for 5 min and Pd(dppf)Cl₂ (0.301 g, 0.411 mmol) was added. The reaction mixture was heated at 90° C. for 18 h. The reaction mixture was quenched with water (10 mL). This was extracted with EtOAc (2×20 mL). The combined organic layer was washed with brine (10 mL), dried over anhydrous Na₂SO₄, filtered and concentrated under reduced pressure. The resulting crude residue was purified by MPLC (using manually packed SiO₂ cartridge, 100-200 mesh size; 30% EtOAc in hexanes) to get 6 as white semi-solid.

Yield: 900 mg (60%)

### Step 6:

To a stirred solution of 7 (300 mg, 0.715 mmol) and 6 (311 mg, 1.073 mmol) in a mixture of acetonitrile (4 ml) and water (2 mL), was added potassium carbonate (297 mg, 2.146 mmol) at room temperature. The resulting reaction mixture was purged with nitrogen for 5 min and PdCl₂(dtbpf) (46.6 mg, 0.072 mmol) was added. The reaction mixture was heated at 90° C. for 16 h. The reaction mixture was quenched with water (10 mL). This was extracted with EtOAc (2×10 mL). The combined organic layer was washed with brine (10 mL), dried over anhydrous Na₂SO₄, filtered and the filtrate was concentrated under reduced pressure to get the crude residue. The crude residue was purified by MPLC (using manually packed SiO₂ cartridge, 100-200 mesh; 100% EtOAc in hexanes) to get 8 as brown gum.

Yield: 200 mg (53%)

LCMS: Calculated for C30H34N2O5 is 502.6, Observed: 503.2 [M+1]⁺

### Step 7:

To a stirred solution of 8 (180 mg, 0.358 mmol) in MeOH (8 mL), was added p-toluenesulfonic acid monohydrate (204 mg, 1.074 mmol) at 0° C. The reaction mixture was slowly warmed to room temperature and stirred for 2 h. The reaction mixture was quenched with aqueous 10% NaHCO3 (10 mL). This was extracted with DCM (2×10 mL). The combined organic layer was washed with brine (10 mL), dried over anhydrous Na₂SO₄, filtered and the filtrate was concentrated under reduced pressure to get the crude residue. The crude residue was purified by reversed-phase preparative HPLC (Column: X-SELECT (C18, 19×250) mm, 5 μm; Eluents: 0.1% ammonium bicarbonate in water and ACN) to obtain Compound 136 as a white solid.

Yield: 20 mg (13%)

LCMS: Calculated for C25H26N2O4 is 418.4, Observed: 419.3 [M+1]⁺

### Example A84: Synthesis of Compound 137

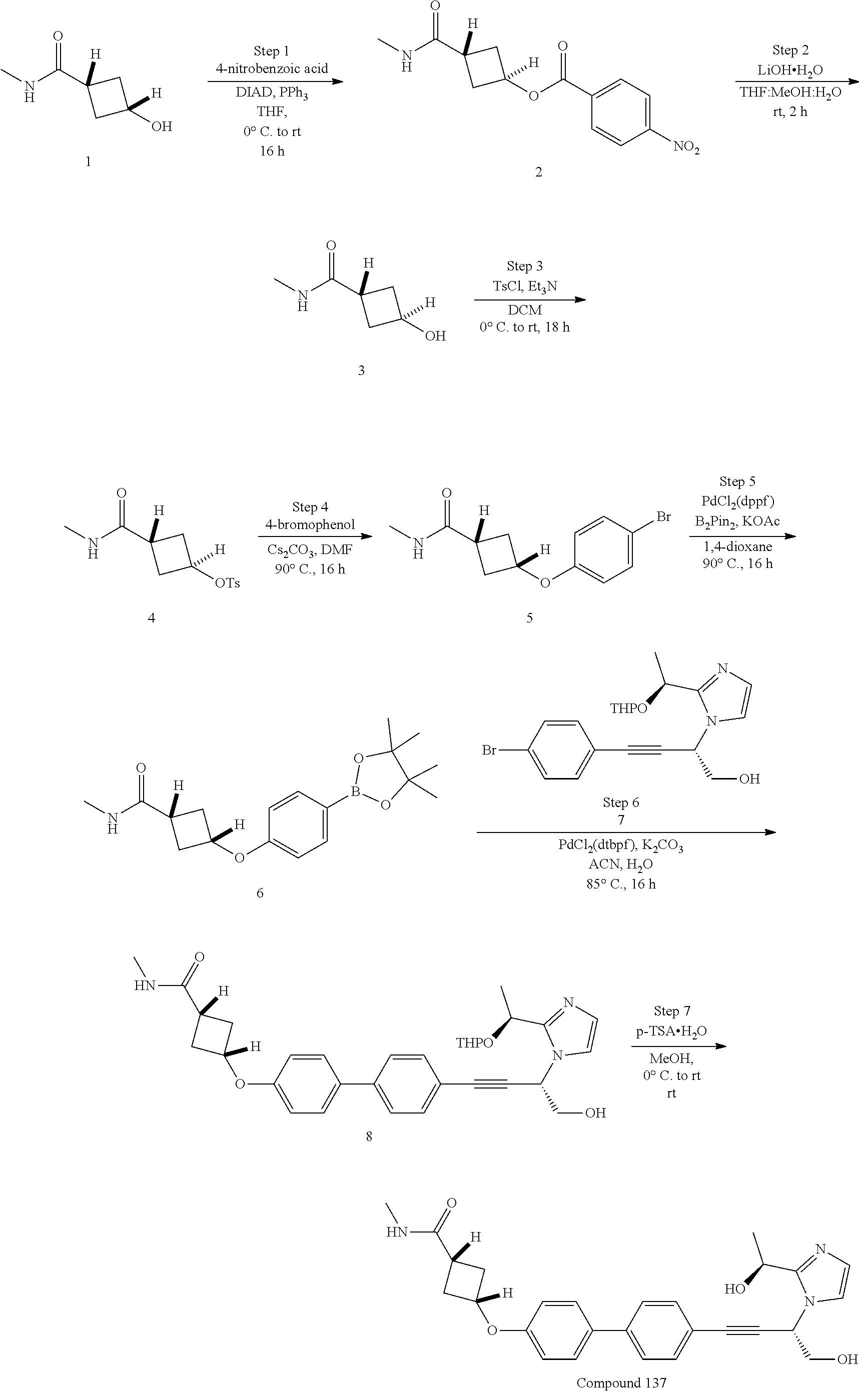

### Step 1:

To a stirred solution of 1 (2.5 g, 19.36 mmol), 4-nitrobenzoic acid (3.56 g, 21.29 mmol) and triphenylphosphine (6.09 g, 23.23 mmol) in THF (50 mL), was added DIAD (5.11 mL, 29.0 mmol) (in drops!) at 0° C., and the reaction mixture stirred for 16 h upon attaining room temperature. The reaction mixture was quenched with cold water (25 mL) at 0° C., extracted with EtOAc (3×50 mL). The combined organic extract was washed with brine solution (30 mL), dried over anhydrous sodium sulphate, filtered, and concentrated under reduced pressure. The crude residue, thus obtained, was purified by using MPLC (manually packed SiO₂ cartridge, 230-400 mesh size; 60% of EtOAc in hexanes) to afford 2 as an off-white solid.

Yield: 2.8 g (39%)

### Step 2:

To a stirred solution of 2 (2.8 g, 8.05 mmol) in MeOH (7.5 mL), THF (15 mL) and water (7.5 mL), was added LiOH·H₂O (1.013 g, 24.15 mmol) at 10° C. and the reaction mixture stirred for 2 h upon attaining room temperature. The volatiles were removed under reduced pressure and the crude residue, thus obtained, was purified by using MPLC (manually packed SiO₂ cartridge, 230-400 mesh size; 6% MeOH in DCM) to afford 3 as semisolid.

Yield: 1.2 g (92%)

### Step 3:

To the stirred solution of 3 (1.2 g, 8.36 mmol) and DMAP (0.204 g, 1.672 mmol) in DCM (25 mL), were added TEA (2.331 mL, 16.72 mmol) and tosyl chloride (3.19 g, 16.72 mmol) at 0° C. and the reaction mixture stirred at room temperature for 18 h. The reaction mixture was quenched with water (10 mL) and extracted with DCM (3×25 mL). The combined organic extract was washed with brine solution (10 mL), dried over anhydrous sodium sulphate, filtered, and concentrated under reduced pressure. The crude residue, thus obtained, was purified by using MPLC (manually packed SiO₂ cartridge; 230-400 mesh size; 90% EtOAc in hexanes) to obtain 4 as an off-white solid.

Yield: 1.7 g (70%)

LCMS: Calculated for C₁₃H₁₇NO₄S is 283.34, Observed: 284.1 [M+1]⁺

### Step 4:

To a stirred solution of 4 (1.2 g, 4.24 mmol) in DMF (15 mL), were added 4-bromophenol (0.733 g, 4.24 mmol) and Cs₂CO₃ (2.76 g, 8.47 mmol) at room temperature and the resulting reaction mixture heated to 90° C. for 16 h. The reaction mixture was then cooled to room temperature and quenched with ice cold water (100 mL) and stirred for 10 min. The solid formed was filtered, and washed with cold water (2×25 mL) and dried under vacuum to afford 5 as an off-white solid.

Yield: 0.95 g (77%)

LCMS: Calculated for C₁₂H₁₄BrNO₂ is 284.15. Observed: 284.1 [M]⁺ and 286.1 [M+2]⁺

SFC purity=98.5%; tR=2.03 min (Column: Lux Amylose-1; Eluents: 0.5% isopropyl amine in MeOH and CO2).

The identity of cis-geometry of 5 was established through the nOe enhancement observed at 4.60 ppm (methine a- to bromophenoxy) upon irradiation of the other methine at 2.60 ppm (methine a- to carboxamide).

### Step 5:

To a stirred solution of 5 (900 mg, 3.17 mmol) in 1,4-dioxane (25 mL), were added potassium acetate (622 mg, 6.33 mmol) and bis(pinacolato)diboron (1.2 g, 4.75 mmol) at room temperature, and a stream of nitrogen passed through the reaction mixture for 10 min. To this reaction mixture. PdCl₂(dppf) (232 mg, 0.317 mmol) was added, and bubbling of nitrogen continued for another 5 min. The reaction mixture was stirred at 90° C. for 16 h. The reaction mixture was cooled to room temperature, quenched with water (20 mL) and extracted with EtOAc (3×40 mL). The combined organic extract was washed with brine solution (20 mL), dried over anhydrous sodium sulphate, filtered, and concentrated under reduced pressure. The crude residue thus obtained was purified by using MPLC (manually packed SiO2 cartridge; 100-200 mesh size; 45% EtOAc in hexanes) to get 6 as an off-white solid.

Yield: 650 mg (57%)

LCMS: Calculated for C₁₈H₂₆BNO₄ is 331.2. Observed: 332.2 [M+1]⁺

### Step 6:

To the stirred solution of 7 (500 mg, 1.192 mmol) in ACN (5 mL) and water (2.5 mL), were added 6 (474 mg, 1.431 mmol) and K₂CO₃ (494 mg, 3.58 mmol) at room temperature, and a stream of nitrogen passed through the reaction mixture for 5 min. To this reaction mixture, PdCl₂(dtbpf) (78 mg, 0.119 mmol) was added at room temperature and the resulting reaction mixture heated at 85° C. for 16 h. The reaction mixture was cooled to room temperature and quenched with ice cold water (30 mL) and extracted with 5% MeOH in DCM (3×50 mL). The combined organic extract was washed with brine solution (30 mL), dried over anhydrous sodium sulphate, filtered, and concentrated under reduced pressure. The crude residue, thus obtained, was purified by using MPLC (manually packed SiO2 cartridge; 230-400 mesh size; 4% MeOH in DCM) to get 8 as brown solid.

Yield: 290 mg (43%)

LCMS: Calculated for C₃₂H₃₇N₃O₅ is 543.66. Observed: 544.3 [M+1]⁺

### Step 7:

To the stirred solution of 8 (280 mg, 0.515 mmol) in MeOH (40 mL), was added p-TSA·H₂O (294 mg, 1.545 mmol) at 0° C. and the resulting reaction mixture stirred at room temperature for 4 h. The reaction mixture was quenched with sat. NaHCO₃ solution (12 mL) at 0° C. and extracted with DCM (3×100 mL). The combined organic extract was washed with sat. NaHCO₃ solution (2×10 mL) followed by brine solution (10 mL), dried over anhydrous sodium sulphate, filtered, and concentrated under reduced pressure. The resulting crude residue was purified by using MPLC (manually packed SiO2 cartridge, 230-400 mesh size; 6% MeOH in DCM) to afford Compound 137 as an off-white solid.

Yield: 90 mg (37%)

LCMS: Calculated for C₂₇H₂₉N₃O₄ is 459.55. Observed: 460.2 [M+1]⁺

1H-NMR (400 MHz, DMSO-d6): δ 7.81 (d, J=4.40 Hz, 2H), 7.65 (d, J=8.40 Hz, 2H), 7.62 (d, J=8.80 Hz, 2H), 7.51 (d, J=8.40 Hz, 2H), 7.35 (d, J=1.20 Hz, 1H), 6.95 (s, 1H), 6.84 (d, J=1.20 Hz, 1H), 5.69 (t, J=6.00 Hz, 1H), 5.53 (t, J=5.60 Hz, 1H, exchanges with D2O), 5.36 (d, J=5.60 Hz, 1H, exchanges with D2O), 4.95-4.92 (m, 1H), 4.69-4.66 (m, 1H), 3.86 (t, J=6.00 Hz, 2H), 2.68-2.50 (m, 6H, merges with solvent peak), 2.20-2.13 (m, 2H), 1.51 (d, J=6.40 Hz, 3H).

SFC purity=98.9%; tR=2.19 min (Column: I Cellulose-Z; Eluents: 0.5% isopropyl amine in MeOH and CO2). cis-geometry at tail part. Mixture of diastereomers; racemic at tail.

### Example A85: Synthesis of Compound 138

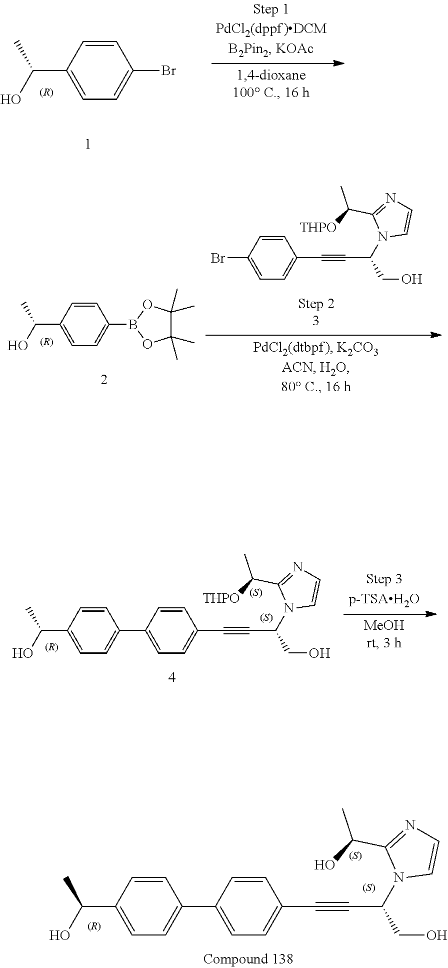

### Step-1:

To a solution of (R)-1-(4-bromophenyl)ethan-1-ol (1, 2 g, 9.95 mmol) in 1,4-dioxane (30 mL), were added potassium acetate (2.93 g, 29.8 mmol) and bis(pinacolato)diboron (3.79 g, 14.92 mmol) at room temperature and the resulting mixture purged with nitrogen for 10 min. To this reaction mixture, PdCl₂(dppf)·CH₂Cl₂ adduct (0.812 g, 0.995 mmol) was added and the purging continued for another 2 min. The resulting reaction mixture was stirred at 100° C. for 16 h. After the reaction mixture was cooled to room temperature; the inorganic solids were filtered through the Celite pad and washed with EtOAc (100 mL). The combined filtrate was concentrated under reduced pressure. The resulting crude mass was purified by using MPLC (manually packed SiO₂ cartridge, 100-200 mesh size; 10% EtOAc in hexanes) to afford 2 as a pale-yellow solid.

Yield: 2.4 g (88%)

### Step-2:

To a stirred solution of 3 (500 mg, 1.192 mmol) in acetonitrile (10 mL) and water (10 mL), were added 2 (444 mg, 1.789 mmol) and K₂CO₃ (494 mg, 3.58 mmol) at room temperature and the reaction mixture purged with nitrogen for 5 min. To this reaction mixture, PdCl₂(dtbpf) (78 mg, 0.119 mmol) was added and the purging continued for another 2 min. The resulting reaction mixture was stirred at 80° C. for 16 h. The reaction mixture was then quenched with water (25 mL), extracted with 10% MeOH in DCM (25 mL×2). The combined organic layer was washed with brine solution (25 mL), dried over anhydrous Na₂SO₄, filtered and concentrated under reduced pressure. The resulting crude product was purified by using MPLC (manually packed SiO₂ cartridge, 230-400 mesh size; 10% MeOH in DCM) to afford 4 as yellow solid. LCMS showed 56% purity; the product was taken to the next step without further purification.

Yield: 350 mg

LC-MS: Calculated for C₂₈H₃₂N₂O₄ is 460.57, Observed: 461.2 [M+1]⁺

### Step-3:

To a stirred solution of 4 (350 mg, 0.760 mmol) in MeOH (20 mL), p-toluenesulfonic acid monohydrate (434 mg, 2.28 mmol) was added at 0° C. and the reaction mixture warmed to room temperature and stirred for 3 h. The volatiles were evaporated under reduced pressure. The resulting residue was basified with 10% NaHCO₃ solution (30 mL) and extracted with 10% MeOH in DCM (50 mL×2). The combined organic layer was washed with brine (30 mL), dried over anhydrous Na₂SO₄, filtered and concentrated under reduced pressure. The crude mass, thus obtained, was purified by reversed phase column chromatography using MPLC (Column: Redisep Gold, C-18 silica gel; Eluents: 0.01 mM ammonium bicarbonate and ACN) to get 90 mg of product. 1H NMR showed extraneous peaks with SFC purity 77.3%. The product was re-purified by SFC (Column: LUX-I-A3 (250*20) mm, 5 μm; Eluents: CO₂:0.5% isopropyl amine in MeOH [65:35]). The fractions were concentrated and resulting residue dissolved in DCM (50 mL) and washed with water (20 mL). The organic layer was dried over anhydrous Na₂SO₄, filtered, concentrated and lyophilized to afford Compound 138 as an off-white solid.

Yield=20 mg (7%)

LC-MS: Calculated for C₂₃H₂₄N₂O₃ is 376.45, Observed: 377.2 [M+1]⁺

1H-NMR (400 MHz, DMSO-d6): δ 7.69 (d, J=8.00 Hz, 2H), 7.64 (d, J=8.00 Hz, 2H), 7.54 (d, J=8.00 Hz, 2H), 7.44 (d, J=8.00 Hz, 2H), 7.36 (d, J=0.80 Hz, 1H), 6.84 (s, 1H), 5.70 (t, J=5.60 Hz, 1H), 5.59 (br s, 1H, exchanges with D2O), 5.42 (br s, 1H, exchanges with D2O), 5.21 (d, J=3.60 Hz, 1H, exchanges with D2O), 4.94 (m, 1H), 4.76 (br s, 1H), 3.87 (d, J=6.00 Hz, 2H), 1.51 (d, J=6.80 Hz, 3H), 1.35 (d, J=6.40 Hz, 3H).

SFC: 95.7%; tR=1.85 min (Column: Whelk-(R,R); Eluents: CO2 and 0.5% isopropyl amine in MeOH). SFC purity=95.7%

### Example A86: Synthesis of Compound 139

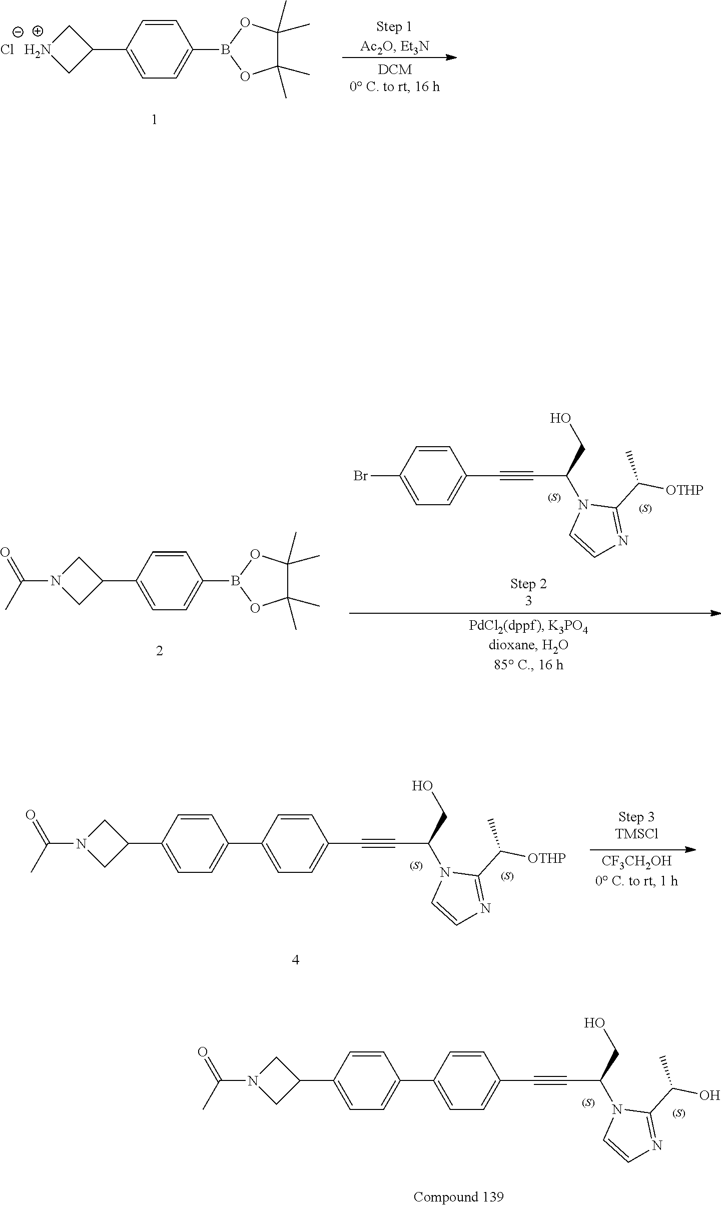

### Step-1:

To a stirred solution of 1 (3.7 g, 12.52 mmol) in DCM (50 mL), were added triethylamine (5.2 mL, 37.5 mmol) and acetic anhydride (2.4 mL, 25.03 mmol) at 0° C. and the reaction mixture stirred at room temperature for 16 h. The reaction was quenched with water (15 mL) and extracted with DCM (2×100 mL). The combined organic extract was washed with brine solution (20 mL) and dried over anhydrous Na₂SO₄, filtered and concentrated under reduced pressure. The resulting crude mass was purified by using MPLC (manually packed SiO2 cartridge, 100-200 mesh size; 35% EtOAc in hexanes) to afford 2 as a brown solid.

Yield: 850 mg (21%)

Calculated for C₁₇H₂₄BNO₃ is 301.19, Observed: 302.0 [M+1]⁺

### Step-2:

To a stirred solution of 3 (450 mg, 1.073 mmol) in dioxane (10 mL) and water (2 mL), were added 2 (388 mg, 1.288 mmol) and potassium phosphate tribasic (683 mg, 3.22 mmol) at room temperature. The reaction mixture was purged with nitrogen for 5 min. To this reaction mixture, PdCl2(dppf) (79 mg, 0.107 mmol) was added and the purging continued for 2 min. The resulting reaction mixture was stirred at 85° C. for 16 h. The reaction mixture was then cooled to room temperature and the inorganic solids were filtered through a Celite pad and washed with 10% MeOH in DCM (50 mL). The filtrate was blended and concentrated under reduced pressure. The resulting crude mass was purified by using MPLC (manually packed SiO2 cartridge, 100-200 mesh size; 10% MeOH in DCM) to afford 4 as a brown gum.

Yield: 380 mg (64%)

Calculated for C₃₁H₃₅N₃O₄ is 513.64, Observed: 514.2 [M+1]⁺

### Step-3:

To a stirred solution of 4 (0.38 g, 0.740 mmol) in 2,2,2-trifluoroethanol (15 mL), was added TMSCl (1M in THF) (0.740 mL, 0.740 mmol) at 0° C. and the reaction mixture stirred at room temperature for 1 h. The volatiles in the reaction mixture were removed under reduced pressure. The crude mass, thus obtained, was purified by reverse phase column chromatography (Column: Redisep Gold; C18 SiO₂; Eluents: 10 mM ammonium bicarbonate in water and ACN) to afford Compound 139 as a white solid.

Yield: 110 mg (34%)

Calculated for C₂₆H₂₇N₃O₃ is 429.52, Observed: 430.3 [M+1]⁺

1H-NMR (400 MHz, DMSO-d6): δ 7.72-7.69 (m, 4H), 7.55 (d, J=8.40 Hz, 2H), 7.48 (d, J=8.00 Hz, 2H), 7.36 (app d, J=1.20 Hz, 1H), 6.84 (app d, J=0.80 Hz, 1H), 5.67 (t, J=6.00 Hz, 1H), 5.54 (t, J=5.60 Hz, 1H, exchanges with D2O), 5.38 (d, J=5.60 Hz, 1H, exchanges with D2O), 4.96-4.93 (m, 1H), 4.53 (t, J=8.40 Hz, 1H), 4.26 (t, J=8.80 Hz, 1H), 4.17-4.14 (m, 1H), 3.90-3.82 (m, 4H), 1.81 (s, 3H), 1.51 (d, J=6.40 Hz, 3H).

SFC: 96.5%, tR=3.28 min (Column: Whelk-(R,R); Eluents: CO2 and 0.5% isopropyl amine in MeOH)

### Example A87: Synthesis of Compound 140

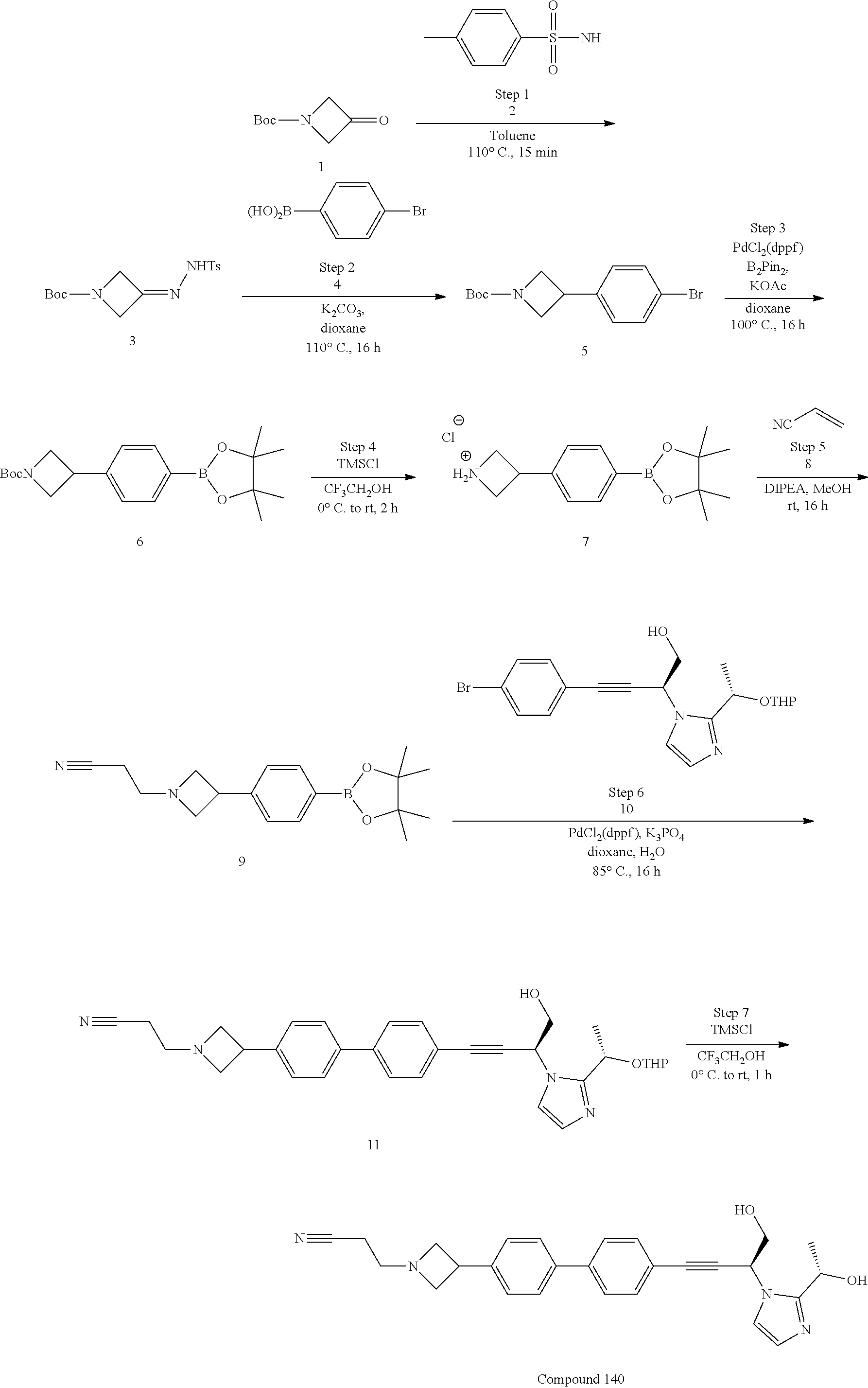

### Step 1:

To a stirred solution of 1-Boc-3-azetidinone (1, 15 g, 83 mmol) in MeOH (240 mL), was added 4-methylbenzenesulfonohydrazide (2, 15.50 g, 83 mmol) at room temperature and the resulting reaction mixture heated at 110° C. for 15 min. The precipitate obtained was filtered through Buchner funnel to afford 3 as a white solid.

Yield=27 g (90%; based on NMR purity)

Calculated for C₁₅H₂₁N₃O₄S is 339.41, Observed: 240.2 [(M-Boc)+1]⁺

### Step 2:

To a stirred solution of 3 (27 g, 80 mmol) in dioxane (398 mL), were added (4-bromophenyl)boronic acid (4, 19.17 g, 95 mmol) and potassium carbonate (33.0 g, 239 mmol) at room temperature and the resulting reaction mixture heated at 110° C. for 16 h. The reaction mixture was then quenched with water (50 mL) and extracted with EtOAc (200 mL×2). The combined organic layer was washed with brine (50 mL), dried over anhydrous Na₂SO₄, filtered and concentrated under reduced pressure. The resulting crude residue was purified by using MPLC (manually packed SiO₂ cartridge, 230-400 mesh size; 15% EtOAc in hexanes) to obtain 5 as a colorless liquid.

Yield=9 g (33%)

Calculated for C₁₄H₁₈BrNO₂ is 312.21, Observed: 212.3 [M-Boc]⁺ and 214.3 [(M-Boc)+2]⁺

### Step 3:

To a stirred solution of 5 (7.5 g, 24.02 mmol) in dioxane (100 mL), were added potassium acetate (7.07 g, 72.1 mmol) and bis(pinacolato)diboron (9.15 g, 36.0 mmol) at room temperature and the resulting mixture purged with nitrogen for 5 min. To this reaction mixture, PdCl₂(dppf) (1.758 g, 2.402 mmol) was added and stirred at 100° C. for 16 h. The reaction mixture was then quenched with water (50 mL) and extracted with EtOAc (200 mL×2). The combined organic layer was dried over anhydrous Na₂SO₄, filtered and concentrated under reduced pressure. The resulting crude residue was purified by using MPLC (manually packed SiO₂ cartridge, 230-400 mesh size; 10% EtOAc in hexanes) to obtain 6 as a yellow solid.

Yield=8 g (84%)

Calculated for C₂₀H₃₀BNO₄ is 359.27, Observed: 260.2 [(M-Boc)+1]⁺

### Step 4:

To a stirred solution of 6 (6 g, 16.70 mmol) in 2,2,2-trifluoroethanol (80 mL), was added TMSCl (2.135 mL, 16.70 mmol) at 0° C. and the reaction mixture stirred at room temperature for 2 h. The volatiles were removed under reduced pressure to afford 7 as a white solid. The crude product was taken as such to the next step.

Yield=6.7 g (crude)

Calculated for C15H23BNO2⁺ is 260.16, Observed: 260.2 [M]⁺

### Step 5:

To a stirred solution of 7 (3 g, 10.15 mmol) in MeOH (40 mL), were added acrylonitrile (8, 0.862 g, 16.24 mmol) and DIPEA (5.3 mL, 30.4 mmol) at 0° C., and the resulting reaction mixture stirred at room temperature for 16 h. The volatiles were evaporated under reduced pressure to get the crude residue. To this residue, water (15 mL) was added and extracted with EtOAc (100 mL×2). The combined organic layer was washed with brine (20 mL), dried over anhydrous Na₂SO₄, filtered and concentrated under reduced pressure. The resulting crude residue was purified by using MPLC (manually packed SiO₂ cartridge, 230-400 mesh size; 35% EtOAc in hexanes) to obtain 9 as a yellow gum.

Yield: 1.1 g (32%)

Calculated for C₁₈H₂₅BN₂O₂ is 312.22, Observed: 313.3 [M+1]⁺

### Step 6:

To a stirred solution of 10 (450 mg, 1.073 mmol) in dioxane (10 mL) and water (2 mL), were added 9 (402 mg, 1.288 mmol) and potassium phosphate tribasic (683 mg, 3.22 mmol) at room temperature and the reaction mixture purged with nitrogen for 5 min. To this reaction mixture, PdCl2(dppf) (79 mg, 0.107 mmol) was added and purging continued for another 2 min. The resulting reaction mixture was stirred at 85° C. for 16 h. The reaction mixture was cooled to room temperature. The inorganic solids were filtered through a Celite pad and washed with 10% MeOH in DCM (50 mL). The combined filtrate was concentrated under reduced pressure. The crude mass, thus obtained, was purified by using MPLC (manually packed SiO2 cartridge, 230-400 mesh size; 10% MeOH in DCM) to afford 11 as a brown gum.

Yield: 450 mg (73%)

Calculated for C₃₂H₃₆N₄O₃ is 524.67, Observed: 525.3 [M+1]⁺

### Step 7:

To a stirred solution of 11 (440 mg, 0.839 mmol) in 2,2,2-trifluoroethanol (10 mL), was added TMSCl (1 M in THF, 0.880 mL, 0.880 mmol) at 0° C., and the reaction mixture stirred at room temperature for 1 h. The volatiles in the reaction mixture were removed under reduced pressure. The crude mass, thus obtained, was purified by reverse phase column chromatography (Column: Redisep Gold; C18 SiO₂; Eluents: 10 mM ammonium bicarbonate in water and ACN) to afford Compound 140 as an off-white solid.

Yield: 26 mg (7%)

Calculated for C₂₇H₂₈N₄O₂ is 440.55, Observed: 441.2 [M+1]⁺

1H-NMR (400 MHz, DMSO-d6): δ 7.70 (d, J=8.40 Hz, 2H), 7.66 (d, J=8.40 Hz, 2H), 7.54 (d, J=8.40 Hz, 2H), 7.45 (d, J=8.40 Hz, 2H), 7.36 (d, J=1.20 Hz, 1H), 6.84 (d, J=1.20 Hz, 1H), 5.70 (t, 1H), 5.54 (t, 1H, exchanges with D2O), 5.38 (d, J=5.60 Hz, 1H, exchanges with D2O), 4.94 (m, 1H), 3.87 (t, J=5.20 Hz, 2H), 3.69-3.67 (m, 3H), 3.17-3.16 (m, 2H), 2.69-2.66 (m, 2H), 2.56-2.51 (m, 2H), 1.51 (d, J=6.40 Hz, 3H).

SFC: 90.3%; tR=4.95 min (Column: Whelk-(R,R); Eluents: CO2 and 0.5% isopropyl amine in MeOH)

### Example A88: Synthesis of Compound 141

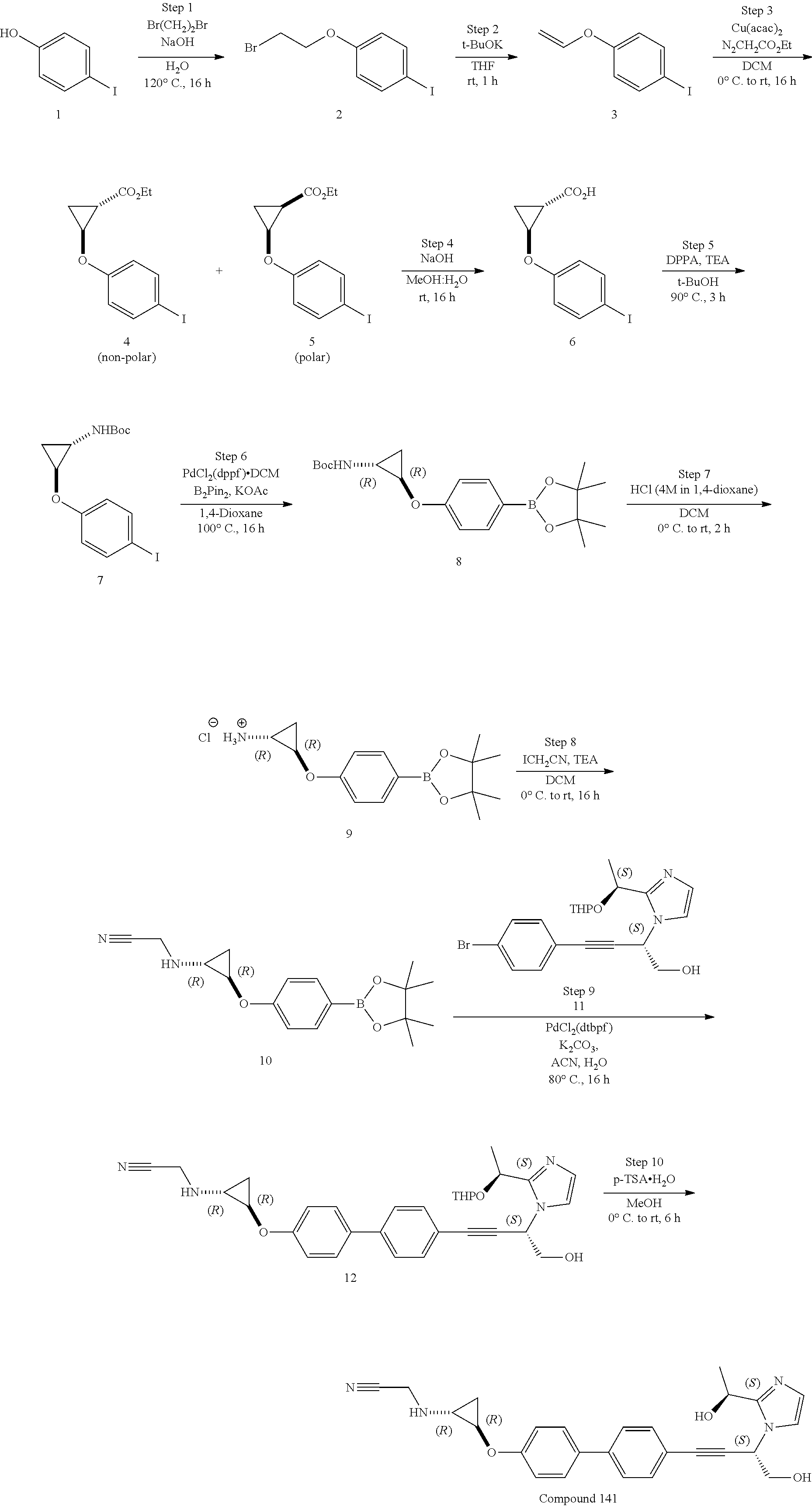

### Step-1:

To a stirred solution of 4-iodophenol (1, 50 g, 227 mmol) in water (270 mL), were added NaOH (19.09 g, 477 mmol) and dibromoethane (213 g, 1136 mmol) and the resulting reaction mixture heated to 120° C. for 16 h. After completion of reaction, the reaction mixture was extracted with EtOAc (2×500 mL). The combined organic layer was dried over anhydrous Na₂SO₄ and concentrated under reduced pressure. The crude product was purified by using MPLC (manually packed cartridge; SiO2 230-400 mesh; 100% Hexanes) to afford 2 as a white solid.

Yield: 43 g (55%)

### Step-2:

To a stirred solution of 2 (21 g, 64.2 mmol) in THE (210 mL), was added t-BuOK (10.81 g, 96 mmol) and stirred at room temperature for 1 h. The reaction was quenched with water (200 mL) and extracted with EtOAc (2×200 mL). The combined organic layer was dried over anhydrous Na₂SO₄ and concentrated under reduced pressure. The resulting crude was purified by using MPLC (manually packed cartridge; SiO2 100-200 mesh; 0-25% EtOAc in hexanes) to afford 3 as a pale-yellow liquid.

Yield: 15 g (90%)

### Step-3:

To a stirred solution of 3 (4.5 g, 18.29 mmol) and copper (II) acetylacetonate (4.79 g, 18.29 mmol) in DCM (50 mL), was added ethyl diazoacetate (10.15 mL, 99 mmol) at 0° C. and the reaction mixture was warmed to room temperature and stirred for 16 h. After completion of reaction, DCM was removed under reduced pressure.

One more batch was carried out using 4.5 g of 3. After completion, both batches mixed for work up and purification.
The crude residue was dissolved in EtOAc (100 mL), washed with water (100 mL), brine solution (50 mL), dried over anhydrous Na₂SO₄, filtered and concentrated under reduced pressure. The resulting crude mass was purified by using MPLC (manually packed cartridge; SiO2 100-200 mesh; 2-5% EtOAc in hexanes) to afford 4 (non-polar) and 5 (polar) as pale-yellow liquids.

Yield: 4=7 g and 5=4 g (contaminated with ethyl diazoacetate); (combined yield for two batches).

The trans- and cis geometry of 4 and 5 was confirmed by nOe studies of the respective hydrolysis products (experimental procedure for hydrolysis can be found in step -4).

### Step-4:

To a stirred solution of 4 (7 g, 21.08 mmol) in mixture of water (30 mL) and MeOH (30 mL), was added NaOH (4.21 g, 105 mmol) at room temperature and the reaction mixture stirred for 16 h. The reaction mixture was then concentrated under reduced pressure. To the resulting residue, water (50 mL) was added and washed with EtOAc (2×100 mL). The aqueous layer was acidified with 1.5 N HCl (pH˜3), extracted with EtOAc (2×50 mL). The combined organic layer was dried over anhydrous Na₂SO₄, filtered and concentrated under reduced pressure. The resulting crude mass was purified by using MPLC (manually packed cartridge; SiO2 100-200 mesh; 0-25% EtOAc in hexanes) to afford 6 as an off-white solid.

Yield: 3.3 g (41%)

### Step-5:

To a stirred solution of 6 (3.3 g, 10.85 mmol) in dry t-BuOH (50 mL), were added TEA (1.664 mL, 11.94 mmol) and diphenylphosphoryl azide (2.57 mL, 11.94 mmol) and the reaction mixture stirred at 90° C. for 3 h. The reaction mixture was cooled to room temperature, quenched with 10% NaHCO3 solution (40 mL) and extracted with EtOAc (2×100 mL). The organic layer was dried over anhydrous Na₂SO₄ and concentrated under reduced pressure. The resulting crude mass was purified by using MPLC (manually packed cartridge; SiO2 100-200 mesh; 0-5% EtOAc in hexanes) to afford 7 as an off-white solid.

Yield: 2.2 g (49%)

### Step-6:

To a stirred solution of 7 (2.2 g, 5.86 mmol) in 1,4-dioxane (30 mL), were added potassium acetate (1.726 g, 17.59 mmol) and bis(pinacolato)diboron (2.233 g, 8.80 mmol) at room temperature and the resulting mixture purged with nitrogen for 10 min. To this reaction mixture, Pd(dppf)Cl2·DCM complex (0.239 g, 0.293 mmol) was added and stirred at 100° C. for 16 h. After the reaction mixture was cooled to room temperature, the inorganic solids were filtered through a Celite pad and washed with EtOAc (50 mL). The combined filtrate was washed with water (25 mL), dried over anhydrous Na₂SO₄, filtered and concentrated under reduced pressure. The resulting crude mass was purified by using MPLC (manually packed cartridge; SiO2 100-200 mesh; 10% EtOAc in hexanes) to obtain 8 as a yellow gum.

Yield: 1.25 g (44%)

LC-MS: Calculated C20H30BNO5 is 375.27, Observed: 276.2 [(M-Boc)+1]⁺

### Step-7:

To a stirred solution of 8 (1 g, 2.66 mmol) in DCM (10 mL), was added HCl (4 M in 1,4 dioxane, 2 mL, 7.99 mmol) at 0° C. and the resulting reaction mixture stirred at room temperature for 2 h. The volatiles were evaporated under reduced pressure to afford 9 as a pale-yellow solid.

Yield=750 mg (87%)

LC-MS: Calculated C15H23BNO3⁺ is 276.16, Observed: 276.2 [M]⁺

### Step-8:

To a solution of 9 (700 mg, 2.246 mmol) in DMF (10 mL), were added triethylamine (0.94 mL, 6.74 mmol) and 2-iodoacetonitrile (0.195 mL, 2.70 mmol) at 0° C. and the resulting reaction mixture stirred at room temperature for 16 h. The reaction mixture was quenched with water (50 mL) and extracted with EtOAc (2×50 mL). The combined organic extract was dried over anhydrous Na₂SO₄, filtered and concentrated under reduced pressure. The resulting crude mass was purified by using MPLC (manually packed cartridge; SiO2 100-200 mesh; 20% EtOAc in hexanes) to afford 10 as a colorless liquid.

LC-MS: Calculated C17H23BN2O3 is 314.19, Observed: 315.2 [M+1]⁺

Yield: 300 mg (34%)

### Step-9:

To a stirred solution of 11 (300 mg, 0.715 mmol) in acetonitrile (10 mL) and water (2.5 mL), were added 10 (300 mg, 0.955 mmol) and K2CO3 (297 mg, 2.146 mmol) at room temperature and the reaction mixture purged with nitrogen for 5 min. To this reaction mixture, PdCl2(dtbpf) (46.6 mg, 0.072 mmol) was added and the purging continued for another 2 min. The resulting reaction mixture was heated at 80° C. for 16 h. The reaction mixture was quenched with water (20 mL), extracted with 10% MeOH in DCM (2×20 mL). The combined organic layer was washed with brine solution (20 mL), dried over anhydrous Na2SO4, filtered and concentrated under reduced pressure. The resulting crude product was purified by using MPLC (manually packed SiO2 cartridge, 230-400 mesh size; 5% MeOH in DCM) to afford 12 as a brown gum.

Yield: 180 mg (39%)

LC-MS: Calculated for C31H34N4O4 is 526.64, Observed: 527.3 [M+1]⁺

### Step-10:

To a stirred solution of 12 (160 mg, 0.304 mmol) in MeOH (5 mL), was added p-toluenesulfonic acid monohydrate (173 mg, 0.911 mmol) at 0° C. and the reaction mixture stirred at room temperature for 6 h. The volatiles were evaporated under reduced pressure; the resulting residue was basified with 10% NaHCO3 solution (20 mL) and extracted with 10% MeOH in DCM (30 mL×2). The combined organic extract was dried over anhydrous Na₂SO₄, filtered, and concentrated under reduced pressure. The crude thus obtained was purified by reversed phase column chromatography (Column: Redisep Gold, C-18 silica gel; Eluents: 0.01 mM ammonium bicarbonate and ACN) to get 35 mg of product. 1H NMR showed extraneous peaks. The crude compound was repurified by chiral SFC purification (Column: Chiralpak-(250*20) mm, 5 μm; Eluents: CO₂ and 0.5% isopropyl amine in MeOH [70:30]) and lyophilized to obtained Compound 140_Isomer-1 and Compound 140_Isomer-2 as a white solid.

Yield: Compound 140_Isomer-1=14 mg and Compound 140_Isomer-2=9 mg

### Compound 140 Isomer-1

LC-MS: Calculated for C26H26N4O3 is 442.519, Observed: 443.2 [M+1]⁺

1H-NMR: showed extraneous peaks.

SFC: 89.5%; tR=1.71 min (Column: CHIRALPAK-AS-H; Eluents: CO2 and 0.5% isopropyl amine in MeOH)

### Compound 140 Isomer-2

LC-MS: Calculated for C26H26N4O3 is 442.52, Observed: 443.3 [M+1]⁺

1H-NMR (400 MHz, DMSO-d6): δ 7.67-7.64 (m, 4H), 7.52 (d, J=8.4 Hz, 2H), 7.36 (d, J=1.2 Hz, 1H), 7.14 (d, J=8.8 Hz, 2H), 6.84 (d, J=1.2 Hz, 1H), 5.70 (t, J=6.0 Hz, 1H), 5.53 (t, J=5.6 Hz, 1H, exchanges with D2O), 5.37 (d, J=5.6 Hz, 1H, exchanges with D2O), 4.97-4.92 (m, 1H), 3.87 (t, J=6.0 Hz, 2H), 3.79-3.77 (m, 1H), 3.75-3.51 (m, 2H), 3.46-3.42 (m, 1H), 2.50-2.49 (m, 1H, merges with solvent peak), 1.51 (d, J=6.4 Hz, 31H), 0.97-0.92 (m, 2H).

SFC: 98.9%; tR=2.29 min (Column: CHIRALPAK-AS-H; Eluents: CO2 and 0.5% isopropyl amine in MeOH)

### Example A89: Synthesis of Compound 142

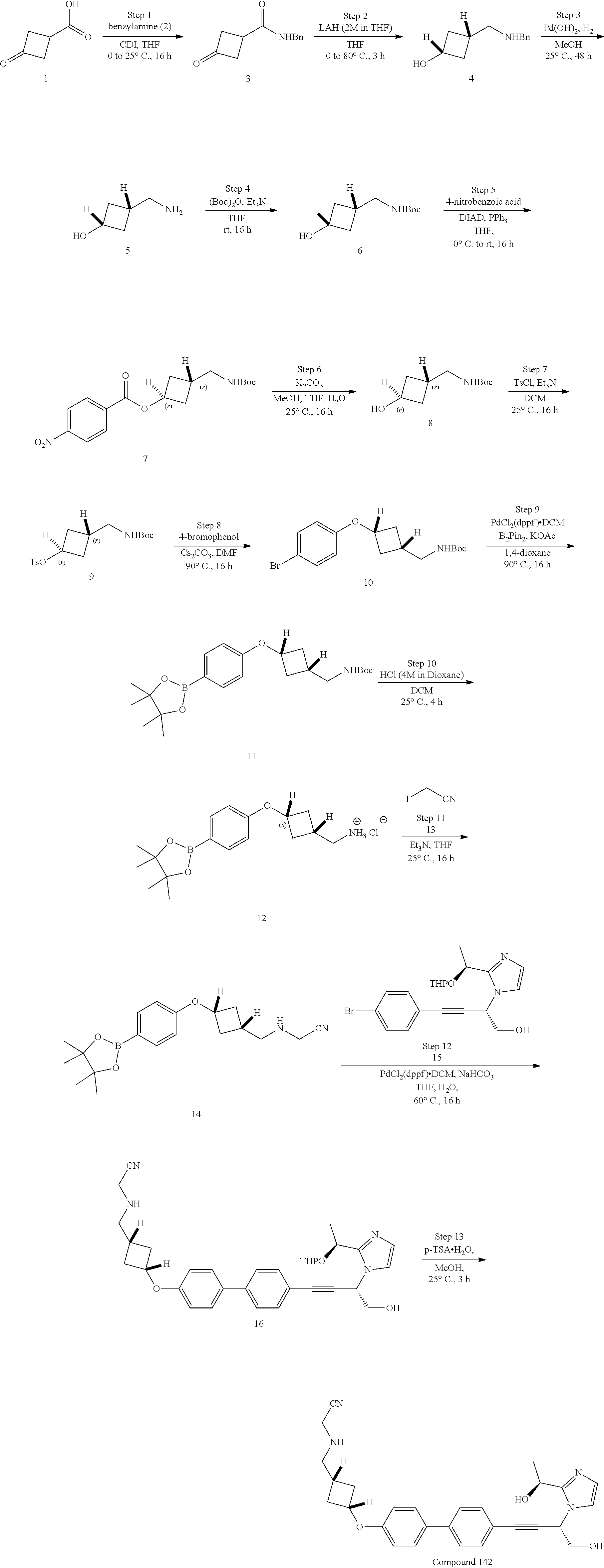

### Step 1:

To a stirred solution of 3-oxocyclobutane-1-carboxylic acid (1, 1.0 g, 8.76 mmol) in THF (10 mL), was added carbonyldiimidazole (1.853 g, 13.15 mmol) at 0° C. and the reaction mixture stirred at room temperature for 1 h. Subsequently, benzylamine (2, 1.409 g, 13.15 mmol) was added and the reaction mixture stirred for 16 h at 25° C. The reaction mixture was concentrated, and the crude residue purified by using MPLC (manually packed SiO2 cartridge, 230-400 mesh; 95% EtOAc in hexane) to afford 3 as a white solid.

Yield: 1 g (52%)

LC-MS: Calculated for C₁₂H₁₃NO₂ is 203.24, Observed: 204.2 [M+1]⁺

### Step-2:

To a stirred solution of 3 (1.0 g, 4.92 mmol) in THF (10 mL), was added lithium aluminum hydride (2 M in THF) (4.92 mL, 9.84 mmol) at 0° C. The reaction mixture was then stirred at 80° C. for 3 h. The reaction was cooled to 0° C., quenched with the addition of saturated Na₂SO₄ solution (8 mL) followed by EtOAc (10 mL), and the resulting reaction mass stirred for 1 h at 25° C. The resulting mixture was filtered through a pad of Celite and washed with THF (20 mL). The filtrate was dried over anhydrous Na₂SO₄, filtered and concentrated under reduced pressure to afford 4 as a pale-brown liquid.

Yield: 1.0 g (89%)

LC-MS: Calculated for C₁₂H₁₇NO is 191.27, Observed: 192.2 [M+1]⁺

### Step-3:

To a stirred solution of 4 (2.5 g, 13.07 mmol) in MeOH (25 mL), was added 10-20% palladium hydroxide on carbon (2.0 g), and the resulting reaction mixture stirred under hydrogen atmosphere (bladder pressure) for 48 h at 25° C. The reaction mixture was filtered through a pad of Celite bed. The bed was washed with MeOH (30 mL), the filtrate combined and concentrated under reduced pressure to afford 5 as a white solid. The crude product was taken to the next step without any further purification.

Yield: 1.1 g (63%)

LC-MS: Calculated for C₅H₁₁NO is 101.15, Observed: 102.2 [M+1]⁺.

One more batch was carried out on 5 g of 4 to get 3.0 g of 5.

### Step-4:

To a stirred solution of 5 (2.5 g, 24.72 mmol) in THF (15 mL), were added Et3N (10.34 mL, 74.1 mmol) and di-tert-butyl dicarbonate (6.47 g, 29.7 mmol) at 0° C., and the reaction mixture stirred at room temperature for 16 h. The reaction mixture was then concentrated under reduced pressure. The crude residue, thus obtained, was purified by using MPLC (manually packed SiO₂ cartridge, 230-400 mesh; 5% MeOH in DCM) to afford 6 as a white solid.

Yield: 4.0 g (69%)

LC-MS: Calculated for C₁₀H₁₉NO₃ is 201.27, Observed: 102.3 [(M-BOC)+1]⁺

### Step-5:

To a stirred solution of 6 (4 g, 19.87 mmol) and 4-nitrobenzoic acid (3.32 g, 19.87 mmol) in THF (40 mL), was added triphenylphosphine (6.26 g, 23.85 mmol) at 0° C., and the resulting reaction mass stirred at 0° C. for 30 min under nitrogen atmosphere. Then DIAD (4.73 mL, 23.85 mmol) was added dropwise at 0° C. and the reaction mixture continued to be stirred for 16 h at 25° C. The reaction was quenched with ice cold water (20 mL) and extracted with EtOAc (3×30 mL). The organic extract was dried over anhydrous sodium sulphate, filtered and concentrated. The crude residue, thus obtained, was purified by using MPLC (manually packed SiO₂ cartridge, 230-400 mesh; 17% EtOAc in hexane) to afford 7 as a white solid. Based on NMR, purity of the product was considered ˜75%. The product was taken to the next step.

Yield: 5.0 g (54%)

### Step-6:

To a stirred solution of 7 (4 g, 10.98 mmol) in THE (13.33 mL) and water (6.67 mL), was added potassium carbonate (3.03 g, 21.95 mmol) at 0° C., and the resulting reaction mixture stirred at 25° C. for 16 h. The reaction mass was concentrated and extracted with EtOAc (3×20 mL). The organic extract was washed with brine (2×10 mL), dried over anhydrous sodium sulphate, filtered and concentrated under reduced pressure. The crude residue, thus obtained, was purified by MPLC (manually packed SiO₂ cartridge, 230-400 mesh; 40% EtOAc in hexane) to afford 8 as a white solid.

Yield: 2.2 g (99%)

LC-MS: Calculated for C₁₀H₁₉NO₃ is 201.27, Observed: 102.3 [(M-BOC)+1]⁺.

### Step-7:

To a stirred solution of 8 (2.2 g, 10.93 mmol) in DCM (30 mL), were added Et3N (4.61 mL, 32.8 mmol) and DMAP (0.134 g, 1.093 mmol) followed by p-toluenesulfonyl chloride (3.13 g, 16.40 mmol) at 0° C. The reaction mixture was stirred at 25° C. for 16 h. The reaction was quenched with water (15 mL) and extracted with DCM (3×10 mL). The combined organic layer was washed with 10% NaHCO₃ (2×5 mL), brine (10 mL), dried over anhydrous Na₂SO₄, filtered and concentrated under reduced pressure. The crude residue thus obtained was purified by using MPLC (manually packed SiO₂ cartridge, 230-400 mesh; 35% EtOAc in hexane) to afford 9 as a white solid.

Yield: 3.15 g (78%)

LC-MS: Calculated for C₁₇H₂₅NO₅S is 355.45, Observed: 256.0 [(M-BOC)+1]⁺

### Step-8:

To a stirred solution of 9 (3.6 g, 10.13 mmol) in DMF (40 mL), were added cesium carbonate (5.27 g, 16.18 mmol) and 4-bromophenol (1.4 g, 8.09 mmol) at room temperature. The reaction mixture was stirred at 90° C. for 16 h. The reaction was quenched with cold water (15 mL) and extracted with EtOAc (3×15 mL). The combined organic layer was washed with brine (2×5 mL), dried over anhydrous Na₂SO₄, filtered and concentrated under reduced pressure. The crude residue thus obtained was purified by using MPLC (manually packed SiO₂ cartridge, 230-400 mesh; 20% EtOAc in hexane) to afford 10 as a white solid.

Yield: 2.9 g (96%)

LC-MS: Calculated for C₁₆H₂₂BrNO₃ is 356.26, Observed: 256.0 [M-BOC]⁺ and 258.0 [(M-BOC)+2]⁺ cis-geometry of 10 was confirmed by nOe studies.

### Step-9:

To a stirred solution of 10 (2.9 g, 8.14 mmol) in 1,4-dioxane (30 mL), were added bis(pinacolato)diboron (2.69 g, 10.58 mmol) and potassium acetate (2.397 g, 24.42 mmol) at room temperature. The reaction mixture was purged with nitrogen for 5 min. To this reaction mixture, PdCl₂(dppf)·CH₂Cl₂ adduct (0.665 g, 0.814 mmol) was added and the purging continued for 10 min. The reaction mixture was then heated at 90° C. for 16 h. The reaction mixture was passed through a pad of Celite and washed with EtOAc (100 mL). The combined filtrate was dried over anhydrous Na₂SO₄, filtered and concentrated under reduced pressure. The resulting crude residue was purified by using MPLC (manually packed SiO₂ cartridge, 230-400 mesh; 14% EtOAc in hexane) to afford 11 as a white solid.

Yield: 3.3 g (83%)

LC-MS: Calculated for C₂₂H₃₄BNO₅ is 403.32, Observed: 304.3 [(M-BOC)+1]⁺

### Step-10:

To a stirred solution of 11 (2 g, 4.96 mmol) in anhydrous DCM (20 mL), was added hydrochloric acid (4 M in dioxane, 2.47 mL, 9.92 mmol) at 0° C., and the resulting mixture stirred at 25° C. for 4 h. The reaction mixture was then concentrated under reduced pressure, and the crude mass triturated with hexane (30 mL) to obtain 12 as an off-white solid.

Yield: 1.5 g (80%)

LC-MS: Calculated for C17H27BNO3⁺ is 304.21, Observed: 304.2 [M]⁺

### Step-11:

To a stirred solution of 12 (1.5 g, 4.42 mmol) in anhydrous THE (15 mL), were added Et3N (1.862 mL, 13.25 mmol) and iodoacetonitrile (13, 0.374 mL, 5.30 mmol) at 25° C., and the reaction mixture stirred for 16 h at the same temperature. The reaction was quenched with water (10 mL) and extracted with EtOAc (3×10 mL). The combined organic layer was washed with brine (20 mL), dried over anhydrous Na₂SO₄, filtered and concentrated under reduced pressure. The crude residue, thus obtained, was purified by using MPLC (manually packed SiO₂ cartridge, 230-400 mesh; 92% EtOAc in hexane) to afford 14 as a colourless gum.

Yield: 1 g (63%)

LC-MS: Calculated for C₁₉H₂₇BN₂O₃ is 342.25, Observed: 343.4 [M+1]⁺

### Step-12:

To a stirred solution of 14 (490 mg, 1.431 mmol) in THF (12 mL) and water (3 mL), were added 15 (500 mg, 1.192 mmol) and NaHCO₃ (200 mg, 2.385 mmol) at room temperature, and the reaction mixture degassed for 10 min. To this reaction mixture, PdCl₂(dppf)·CH₂Cl₂ adduct (97 mg, 0.119 mmol) was added and the purging continued for 10 min. The reaction mixture was then stirred at 60° C. for 16 h. The reaction mixture was filtered through a pad of Celite bed. The bed was further washed with EtOAc (30 mL), the filtrate combined, dried over anhydrous Na₂SO₄, filtered and concentrated under reduced pressure. The crude residue, thus obtained, was purified by using MPLC (manually packed SiO₂ cartridge, 230-400 mesh; 5% MeOH in DCM) to afford 16 as a brown gum. LCMS showed 68% purity; the product was taken to the next step as such without further purification.

Yield: 260 mg (27%)

LC-MS: Calculated for C₃₃H₃₈N₄O₄ is 554.69, Observed: 555.3 [M+1]⁺

### Step-13:

To a stirred solution of 16 (230 mg, 0.415 mmol) in MeOH (5 mL), was added p-toluenesulfonic acid monohydrate (158 mg, 0.829 mmol) at 0° C. and the resulting reaction mixture stirred at 25° C. for 3 h. The reaction mixture was concentrated and basified with sat. NaHCO₃ solution (25 mL). The aqueous layer was extracted with 10% MeOH in DCM (20 mL×3). The combined organic extract was dried over anhydrous Na₂SO₄, filtered and concentrated under reduced pressure. The resulting crude residue was purified by MPLC (Column: RediSep Gold, C-18 reversed phase SiO2; Eluents:10 mM ammonium bicarbonate in water and ACN) to obtain Compound 142 as a white solid.

Yield: 50 mg (25%)

LC-MS: Calculated for C₂₈H₃₀N₄O₃ is 470.57, Observed:471.4 [M+1]⁺.

1H-NMR (400 MHz, DMSO-d6): δ 7.66-7.61 (m, 4H), 7.51 (d, J=8.40 Hz, 2H), 7.35 (app d, J=1.20 Hz, 1H), 6.94 (d, J=8.80 Hz, 2H), 6.84 (app d, J=1.20 Hz, 1H), 5.69 (t, J=6.00 Hz, 1H), 5.53 (t, J=6.00 Hz, 1H, exchanges with D2O), 5.37 (d, J=5.60 Hz, 1H, exchanges with D2O), 4.96-4.92 (m, 1H), 4.66-4.62 (m, 1H), 3.87 (t, J=6.00 Hz, 2H), 3.59 (d, J=6.80 Hz, 2H), 2.65-2.56 (m, 4H), 2.50 (br s, 1H, merges with solvent peak), 2.11-2.09 (m, 1H), 1.78-1.74 (m, 2H), 1.51 (d, J=6.40 Hz, 3H).

SFC: 96.62%; tR=4.06 min (Column: Whelk-(R,R); Eluents: 0.5% isopropyl amine in MeOH and CO₂)

cis-geometry at tail part; mixture of diastereomers; SFC purity=96.62%

### Example A90: Synthesis of Compound 143

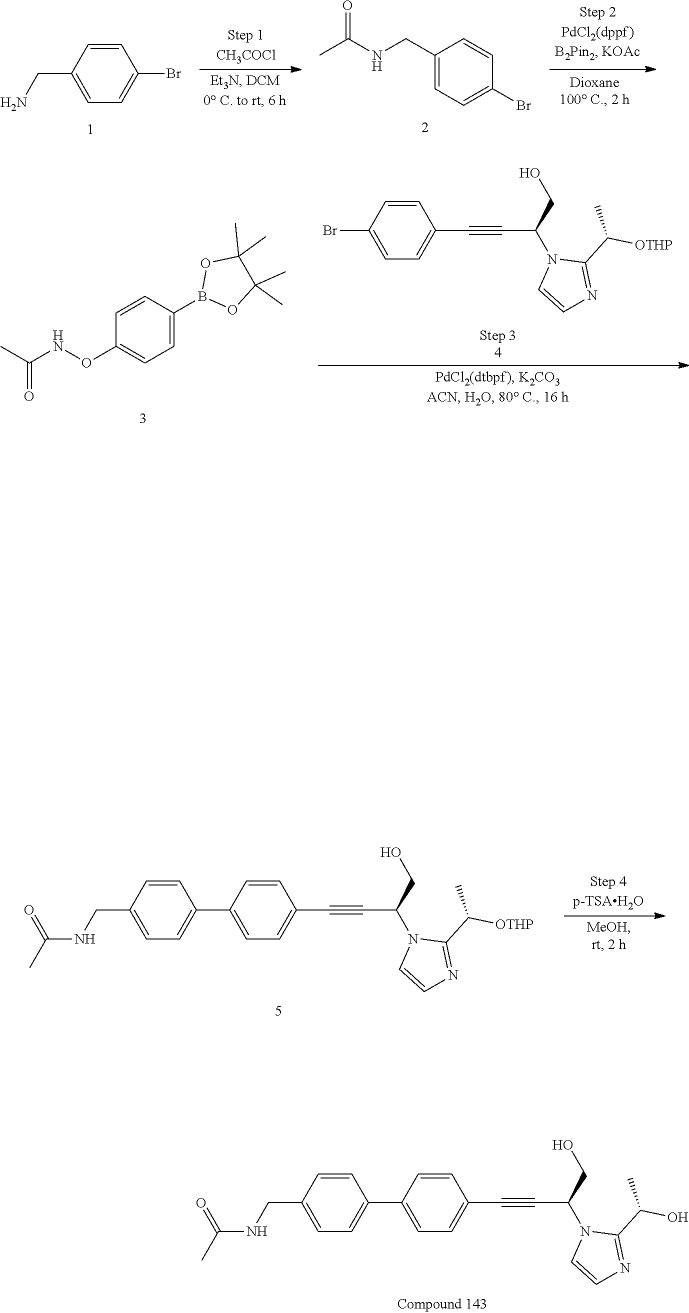

### Step-1:

To a stirred solution of 4-bromobenzyl amine (1, 3 g, 16.12 mmol) in DCM (30 ml) were added triethylamine (6.74 mL, 48.4 mmol) and acetyl chloride (1.720 mL, 24.19 mmol) at 0° C. The resulting reaction mixture was stirred at room temperature for 6 h. After completion, the reaction mixture was quenched with 10% NaHCO₃ solution and extracted with DCM (2×30 mL). The combined organic layer was washed with brine (50 mL), dried over anhydrous Na₂SO₄, filtered and concentrated under reduced pressure. The crude residue was purified by using MPLC (manually packed SiO₂ cartridge, 100-200 mesh size; 70% EtOAc in hexane) to obtain 2 as a colorless solid.

Yield: 3.3 g (87%)

LCMS: Calculated for C₉H₁₀BrNO is 228.09; Observed: 228.2 [M]⁺ and 230.2 [M+2]⁺

### Step-2:

To a stirred solution of 2 (3 g, 13.15 mmol) in dioxane (30 mL), were added bis(pinacolato)diboron (5.01 g, 19.73 mmol) and potassium acetate (3.87 g, 39.5 mmol) at room temperature. Nitrogen gas was bubbled through the reaction mixture for 5 min after which PdCl₂(dppf) (0.481 g, 0.658 mmol) was added and the bubbling of nitrogen continued for 2 min. The reaction mixture was stirred at 100° C. for 2 h. The reaction mixture was diluted with EtOAc (100 mL) and filtered through the Celite bed. The bed was washed with EtOAc (100 mL), the filtrate combined and concentrated under reduced pressure. The crude residue, thus obtained, was purified by using MPLC (manually packed cartridge; SiO₂ 100-200 mesh; 45% EtOAc in hexane) to obtain 3 as a brown solid.

Yield: 3 g (61%)

LCMS: Calculated for C₁₅H₂₂BNO₃ is 275.16; Observed: 276.2 [M+1]⁺

### Step-3:

To a stirred solution of 3 (1.378 g, 5.01 mmol), and 4 (1.5 g, 3.58 mmol) in ACN (10 mL): Water (5 mL), was added potassium carbonate (1.481 g, 10.73 mmol). Nitrogen gas was bubbled through the reaction mixture for 5 min after which PdCl₂(dtbpf) (0.116 g, 0.179 mmol) and the reaction mixture stirred at 80° C. for 16 h. The reaction mixture was quenched with water (50 mL) and extracted with 10% MeOH in DCM (100 mL×2). The combined organic layer was washed with brine (50 mL), dried over anhydrous Na₂SO₄ and concentrated under reduced pressure. The crude residue was purified by MPLC (manually packed SiO₂ cartridge, SiO₂ 230-400 mesh size; 5% MeOH in DCM) to obtain 5 as a brown solid.

Yield: 1 g (56%)

LCMS: Calculated for C₂₉H₃₃N₃O₄ is 487.60; Observed: 488.2 [M+1]⁺

### Step-4:

To a stirred solution of 5 (0.250 g, 0.513 mmol) in MeOH (8 ml), was added p-toluene sulfonic acid monohydrate (0.293 g, 1.538 mmol) at 0° C. and the resulting reaction mixture stirred at room temperature for 2 h. The reaction was quenched with saturated NaHCO₃ solution (10 mL) and extracted with 10% MeOH in DCM (2×50 mL). The combined organic layer was washed with water (50 mL), brine (50 mL), dried over sodium sulfate, filtered and concentrated under reduced pressure. The crude residue was purified by reverse phase column chromatography (Column: Redisep Gold, C-18 SiO2; Eluents: 10 mM ammonium bicarbonate in water and ACN) to obtain Compound 143 as an off-white solid.

Yield: 22 mg (10%)

LCMS: Calculated for C₂₄H₂₅N₃O₃ is 403.1; Observed: 404.3 [M+1]⁺

1H-NMR (400 MHz, DMSO-d6): δ 8.38 (t, J=6.00 Hz, 1H), 7.69 (d, J=8.40 Hz, 2H), 7.65 (d, J=8.00 Hz, 2H), 7.55 (d, J=8.40 Hz, 2H), 7.41 (s, 1H), 7.35 (d, J=8.00 Hz, 2H), 6.91 (s, 1H), 5.71 (t, J=6.00 Hz, 1H), 5.55 (t, J=5.60 Hz, 1H, exchanges with D₂O), 5.46 (d, J=4.80 Hz, 1H, exchanges with D₂O), 5.00-4.94 (m, 1H), 4.29 (d, J=6.00 Hz, 2H), 3.88 (t, J=6.00 Hz, 2H), 1.89 (s, 3H), 1.51 (d, J=6.80 Hz, 3H) SFC: 97.4%; tR=2.49 min (Column: I Cellulose-Z; Eluents: CO2 and 0.5% isopropyl amine in MeOH). Single isomer with SFC purity 97.4%

### Example B-1: Parenteral Pharmaceutical Composition

To prepare a parenteral pharmaceutical composition suitable for administration by injection (subcutaneous, intravenous), 1-100 mg of a water-soluble salt of a compound Formulas (I), (II), (III), (IV), (V), (VI), (VII), (VIII), (IX), (X), (XI), or (XII), or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Example B-2: Oral Solution

To prepare a pharmaceutical composition for oral delivery, a sufficient amount of a compound of Formulas (I), (II), (III), (IV), (V), (VI), (VII), (VIII), (IX), (X), (XI), or (XII), or a pharmaceutically acceptable salt thereof, is added to water (with optional solubilizer(s), optional buffer(s) and taste masking excipients) to provide a 20 mg/mL solution.

### Example B-3: Oral Tablet

A tablet is prepared by mixing 20-50% by weight of a compound of Formulas (I), (II), (III), (IV), (V), (VI), (VII), (VIII), (IX), (X), (XI), or (XII), or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example B-4: Oral Capsule

To prepare a pharmaceutical composition for oral delivery, 10-500 mg of a compound of Formulas (I), (II), (III), (IV), (V), (VI), (VII), (VIII), (IX), (X), (XI), or (XII) or a pharmaceutically acceptable salt thereof, is mixed with starch or other suitable powder blend. The mixture is incorporated into an oral dosage unit such as a hard gelatin capsule, which is suitable for oral administration.

In another embodiment, 10-500 mg of a compound of Formulas (I), (II), (III), (IV), (V), (VI), (VII), (VIII), (IX), (X), (XI), or (XII), or a pharmaceutically acceptable salt thereof, is placed into Size 4 capsule, or size 1 capsule (hypromellose or hard gelatin) and the capsule is closed.

### II. Biological Evaluation

### Example C-1: In Vitro Assays to Screen Compounds and Metalloprotein Modulators

### Bacterial Susceptibility Testing

Minimal inhibitory concentrations (MIC) were determined by the broth microdilution method in accordance with the Clinical and Laboratory Standards Institute (CLSI) guidelines. In brief, organism suspensions were adjusted to a final inoculum between 3×10⁵ and 7×10⁵ colony-forming units (CFU)/mL. Drug dilutions and inocula were made in sterile, cation adjusted Mueller-Hinton Broth (Beckton Dickinson). In wells, an inoculum volume of 100 μL was mixed to 2 μL of DMSO with 2-fold serial dilutions of drug. All inoculated microdilution trays were incubated in ambient air at 35° C. for 18-24 h. Following incubation, the lowest concentration of the drug that prevented visible growth (OD600 nm<0.05) was recorded as the MIC. Performance of the assay was monitored by the use of laboratory quality -control strains and compounds with defined MIC spectrum, in accordance with CLSI guidelines.

Exemplary in vitro assay data against select bacteria for compounds in embodiments of the disclosure is provided in Table A. Compounds of the disclosure do not inhibit S. aureus (MIC values>32 μg/mL).

TABLE A

| | E. coli | K. pneumoniae | P. aeruginosa | P. aeruginosa |
|---|---|---|---|---|
| | ATCC25922 | ATCC13883 MIC | ATCC27853 | PAO1 MIC |
| Cmpd No. | MIC (ug/mL) | (ug/mL) | MIC (ug/mL) | (ug/mL) |
| 1 | 0.50 | 2 | 4 | 2 |
| 2 | 1 | 2 | 4 | 2 |
| 3 | 1 | 2 | 2 | 2 |
| 4 | 2 | 4 | 8 | 8 |
| 5 | 2 | 4 | 4 | 4 |
| 6 | 2 | 4 | 4 | 2 |
| 7 | 1 | 4 | 4 | 2 |
| 8 | 2 | 4 | 4 | 2 |
| 10 | 1 | 2 | 8 | 4 |
| 11 | 1 | 4 | 8 | 4 |
| 13 | 2 | 4 | 16 | 4 |
| 14 | 2 | 4 | 8 | 4 |
| 44 | 4 | 8 | 8 | 8 |
| 45 | 1 | 2 | 4 | 2 |
| 46 | 1 | 2 | 4 | 2 |
| 47 | 1 | 4 | 4 | 4 |
| 48 | 1 | 4 | 4 | 2 |
| 49 | 0.5 | 1 | 4 | 2 |
| 50 | 0.5 | 2 | 4 | 2 |
| 51 | 2 | 4 | 8 | 2 |
| 52 | 1 | 2 | 8 | 2 |
| 53 | 1 | 4 | 8 | 4 |
| 54 | 1 | 2 | 8 | 4 |
| 55 | 1 | 4 | 8 | 4 |
| 56 | 1 | 2 | 8 | 2 |
| 57 | 1 | 4 | 2 | 2 |
| 58 | 1 | 4 | 4 | 4 |
| 59 | 1 | 4 | 4 | 2 |
| 60 | 2 | 4 | 4 | 4 |
| 61 | 4 | 8 | 32 | 16 |
| 62 | 4 | 8 | >32 | 16 |
| 63 | 0.50 | 2 | 4 | 2 |
| 64 | 1 | 2 | 8 | 8 |
| 65 | 2 | 4 | 4 | 2 |
| 66 | 1 | 4 | 4 | 2 |
| 67 | 0.50 | 4 | 4 | 2 |
| 68 | 1 | 2 | 4 | 2 |
| 69 | 1 | 2 | 2 | 1 |
| 70 | 2 | 4 | 1 | 1 |
| 71 | 2 | 4 | 4 | 4 |
| 72 | 1 | 2 | 8 | 2 |
| 73 | 2 | 2 | 8 | 2 |
| 74 | 4 | 4 | 2 | 2 |
| 75 | 0.25 | 1 | 4 | 2 |
| 76 | 2 | 16 | 4 | 4 |
| 77 | 0.50 | 2 | 4 | 4 |
| 78 | 1 | 4 | 4 | 4 |
| 79 | 1 | 8 | 4 | 2 |
| 80 | 0.50 | 2 | 4 | 2 |
| 81 | 0.25 | 0.50 | 8 | 2 |
| 82 | 0.25 | 1 | >32 | 4 |
| 83 | 2 | 4 | >32 | 16 |
| 84 | 8 | 32 | 16 | 8 |
| 85 | 1 | 2 | >32 | 16 |
| 86 | 1 | 2 | 16 | 4 |
| 87 | 2 | 4 | 8 | 4 |
| 88 | 1 | 2 | 4 | 2 |
| 89 | 1 | 2 | 8 | 4 |
| 90 | 2 | 4 | 2 | 1 |
| 91 | 2 | 4 | 4 | 2 |
| 92 | 0.75 | 1.5 | 6 | 3 |
| 93 | 0.50 | 1 | 4 | 2 |
| 94 | 1 | 4 | 2 | 2 |
| 95 | 0.50 | 2 | 4 | 2 |
| 96 | 2 | 8 | 8 | 8 |
| 97 | 4 | 8 | 32 | 16 |
| 98 | 0.50 | 2 | 8 | 4 |
| 99 | 1 | 2 | 8 | 4 |
| 100 | 1 | 2 | 2 | 2 |
| 101 | 0.5 | 1 | 4 | 1 |
| 102 | 2 | 4 | 8 | 4 |
| 103 | 1 | 2 | 2 | 2 |
| 104 | 2 | 4 | 2 | 2 |
| 105 | 1 | 1 | 4 | 4 |
| 106 | 0.25 | 0.50 | 4 | 2 |
| 107 | 0.25 | 0.50 | 4 | 2 |
| 108 | 0.38 | >32 | >32 | >32 |
| 109 | 0.62 | 1.5 | >32 | 2 |
| 110 | 0.38 | 1.2 | >32 | >32 |
| 111 | 0.25 | 0.50 | 2 | 1.5 |
| 112 | >32 | >32 | >32 | >32 |
| 113 | 4 | 8 | >32 | 32 |
| 114 | 4 | 8 | >32 | 16 |
| 115 | 1 | 1 | 8 | 2 |
| 116 | 0.50 | 2 | 4 | 1 |
| 117 | 0.12 | 0.25 | 4 | 2 |
| 118 | 0.12 | 0.38 | 4 | 2 |
| 119 | 4 | 8 | >32 | 8 |
| 120 | 1 | 2 | 4 | 2 |
| 121 | 0.50 | 1 | 4 | 1 |
| 122 | 0.50 | 1 | 2 | 2 |
| 123 | 2 | 2 | 4 | 2 |
| 124 | 2 | 4 | >32 | 8 |
| 125 | 1 | 2 | 8 | 2 |
| 126 | 0.50 | 1 | 4 | 2 |
| 127 | 0.25 | 1 | 1 | 1 |
| 128 | 0.25 | 1 | 4 | 2 |
| 129 | 0.50 | 1 | 4 | 2 |
| 130 | 0.25 | 1 | >32 | 2 |
| 131 | 1 | 2 | >32 | 2 |
| 132 | 2 | >32 | 4 | 4 |
| 133 | 1 | 8 | 4 | 4 |
| 134 | 1 | 8 | 8 | 4 |
| 135 | 0.50 | 2 | 2 | 2 |
| 136 | 0.25 | 0.50 | 4 | 2 |
| 137 | 0.50 | 2 | 1 | 0.50 |
| 138 | 1 | 2 | 16 | 4 |
| 139 | 0.50 | 1 | 4 | 1 |
| 140 | 0.25 | 0.50 | 4 | 2 |
| 141 | 1 | 1 | 8 | 2 |
| 142 | 1 | 2 | 4 | 2 |
| 143 | 4 | 4 | 8 | 2 |

### LpxC Binding Assay

IC₅₀ values against E. coli and P. aeruginosa LpxC are determined using a Rapid Fire MS assay as previously described J. Med. Chem. 2012, 55, 1662-1670.

The examples and embodiments described herein are for illustrative purposes only and various modifications or changes suggested to persons skilled in the art are to be included within the spirit and purview of this application and scope of the appended claims.

What is claimed is:

1. A compound of Formula (I): 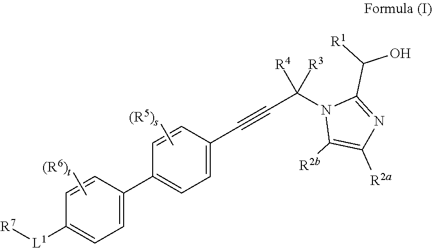
or a pharmaceutically acceptable salt, solvate, or stereoisomer thereof, wherein:
R¹ is C₁-C₄ alkyl;
R^{2a} and R^{2b} are each independently hydrogen, halogen, or C₁-C₄ alkyl;
R³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂;
R⁴ is hydrogen or C₁-C₄ alkyl;
each R⁵ and R⁶ is independently halogen, or C₁-C₄ alkyl;
L¹ is a bond, —(C₁-C₆ alkylene)-, —X¹—, or —X²—(C₁-C₆ alkylene)-, wherein:
X¹ and X² are each selected from: —O—, —N(R⁹)—, —S—, —S(═O)—, —S(═O)₂—, and —S(═O)(═NR⁹)—, wherein:
R⁹ is hydrogen or C₁-C₆ alkyl;
R⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R¹⁰ groups, wherein:
each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein:
each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃;
or two R⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo;
s is 0, 1, or 2;
t is 0, 1, or 2; and
wherein at least one of the following:
(i) s is 1 or 2, and at least one R⁵ is halogen;
(ii) t is 1 or 2, and at least one R⁶ is halogen; and
(iii) the alkyl, heteroalkyl, cycloalkyl, or heterocycloalkyl of R⁷ is substituted by 1, 2, or 3 R¹⁰ groups, and at least one R¹⁰ is halogen.

2. The compound of claim 1, or a pharmaceutically acceptable salt, solvate, or stereoisomer thereof, wherein at least one R⁵, R⁶, or R¹⁰ is —F.

3. The compound of claim 2, or a pharmaceutically acceptable salt, solvate, or stereoisomer thereof, wherein at least one R⁵ or R⁶ is —F.

4. The compound of claim 2, or a pharmaceutically acceptable salt, solvate, or stereoisomer thereof, wherein at least one R¹⁰ is —F.

5. The compound of claim 1, or a pharmaceutically acceptable salt, solvate, or stereoisomer thereof, wherein at least two R⁵, R⁶, or R¹⁰ is —F.

6. The compound of claim 5, or a pharmaceutically acceptable salt, solvate, or stereoisomer thereof, wherein at least two R⁵ or R⁶ is —F.

7. The compound of claim 5, or a pharmaceutically acceptable salt, solvate, or stereoisomer thereof, wherein at least two R¹⁰ is —F.

8. The compound of any one of claims 1-7, or a pharmaceutically acceptable salt, solvate, or stereoisomer thereof, wherein:
R¹ is —CH₃.

9. The compound of any one of claims 1-8, or a pharmaceutically acceptable salt, solvate, or stereoisomer thereof, wherein:
R^{2a} is hydrogen; and
R^{2b} is hydrogen.

10. The compound of any one of claims 1-9 or a pharmaceutically acceptable salt, solvate, or stereoisomer thereof, wherein:
R⁴ is hydrogen.

11. The compound of any one of claims 1-10, or a pharmaceutically acceptable salt, solvate, or stereoisomer thereof, wherein:
each R⁵ is independently —F, —Cl, or —CH₃.

12. The compound of any one of claims 1-11, or a pharmaceutically acceptable salt, solvate, or stereoisomer thereof, wherein:
each R⁵ is —F.

13. The compound of any one of claims 1-12, or a pharmaceutically acceptable salt, solvate, or stereoisomer thereof, wherein:
each R⁶ is —F.

14. The compound of claim any one of claims 1-7, or a pharmaceutically acceptable salt, solvate, or stereoisomer thereof, having the structure of Formula (IIa): 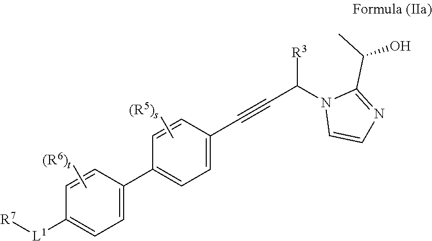
or a pharmaceutically acceptable salt, solvate, or stereoisomer thereof, wherein:
each R⁵ is —F; and
each R⁶ is —F.

15. The compound of claim any one of claims 1-7, or a pharmaceutically acceptable salt, solvate, or stereoisomer thereof, having the structure of Formula (IIIa): 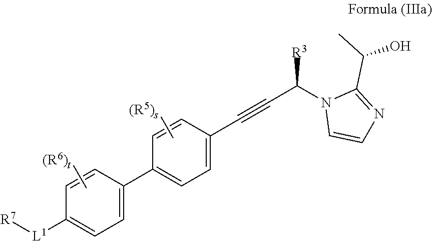
or a pharmaceutically acceptable salt, solvate, or stereoisomer thereof, wherein:
each R⁵ is —F; and
each R⁶ is —F.

16. The compound of any one of claims 1-15, or a pharmaceutically acceptable salt, solvate, or stereoisomer thereof, wherein:
R³ is hydrogen or —(C₁-C₄ alkylene)-OH.

17. The compound of any one of claims 1-15, or a pharmaceutically acceptable salt, solvate, or stereoisomer thereof, wherein:
R³ is —(C₁-C₄ alkylene)-OH.

18. The compound of any one of claims 1-15, or a pharmaceutically acceptable salt, solvate, or stereoisomer thereof, wherein:
R³ is —CH₂OH.

19. The compound of any one of claims 1-18, or a pharmaceutically acceptable salt, solvate, or stereoisomer thereof, wherein:
R⁹ is hydrogen.

20. The compound of any one of claims 1-19, or a pharmaceutically acceptable salt, solvate, or stereoisomer thereof, wherein:
L¹ is a bond, —(C₁-C₄ alkylene)-, —X¹—, or —X²—(C₁-C₄ alkylene)-, wherein:
X¹ and X² are each selected from: —O—, —N(H)—, —S—, —S(═O)—, —S(═O)₂—, and —S(═O)(═NH)—.

21. The compound of any one of claims 1-19, or a pharmaceutically acceptable salt, solvate, or stereoisomer thereof, wherein:
L¹ is —X¹— or —X²—(C₁-C₄ alkylene)-, wherein:
X¹ and X² are each selected from: —O—, —N(H)—, —S—, —S(═O)—, —S(═O)₂—, and —S(═O)(═NH)—.

22. The compound of any one of claims 1-19, or a pharmaceutically acceptable salt, solvate, or stereoisomer thereof, wherein:
L¹ is —X¹— or —X²—(C₁-C₄ alkylene)-, wherein:
X¹ and X² are each selected from: —O—.

23. The compound of any one of claims 1-19, or a pharmaceutically acceptable salt, solvate, or stereoisomer thereof, wherein:
L¹ is —O—.

24. The compound of any one of claims 1-23, or a pharmaceutically acceptable salt, solvate, or stereoisomer thereof, wherein:
R⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R¹⁰ groups, wherein:
each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein:
each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃.

25. The compound of any one of claims 1-23, or a pharmaceutically acceptable salt, solvate, or stereoisomer thereof, wherein:
R⁷ is 4- to 8-membered heterocycloalkyl, where the 4- to 8-membered heterocycloalkyl is unsubstituted or substituted by 1, 2, or 3 R¹⁰ groups, wherein:
each R¹⁰ is independently selected from: halogen, —OR⁸, —N(R⁸)₂, —NHSO₂R⁸, —CH₂CN, C₁-C₄ alkyl, —C(═O)—C₁-C₄ hydroxyalkyl, C₁-C₄ hydroxyalkyl, and C₁-C₄ methoxyalkyl; and
each R⁸ is independently hydrogen, C₁-C₄ alkyl, —C(═O)—C₁-C₄ alkyl, or 4- to 6-membered heterocycloalkyl, wherein the alkyl is unsubstituted or substituted by 1, 2, or 3 groups independently selected from —F, —CN, —OH, —CO₂H, —C(═NH)NH₂, and 5-membered monocyclic heteroaryl which is unsubstituted or substituted by 1 —CONH₂ group.

26. The compound of any one of claims 1-23, or a pharmaceutically acceptable salt, solvate, or stereoisomer thereof, wherein:
R⁷ is 4- to 8-membered heterocycloalkyl, where the 4- to 8-membered heterocycloalkyl is unsubstituted or substituted by 1, 2, or 3 R¹⁰ groups, wherein:
each R¹⁰ is independently selected from: halogen, —OH, —OMe, —N(R⁸)₂, —NHSO₂R⁸, and —CH₂CN; and
each R⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl.

27. The compound of any one of claims 1-23, or a pharmaceutically acceptable salt, solvate, or stereoisomer thereof, wherein:
R⁷ is 4- to 6-membered heterocycloalkyl, where the 4- to 6-membered heterocycloalkyl is unsubstituted or substituted by 1, 2, or 3 R¹⁰ groups, wherein:
each R¹⁰ is independently selected from: —F, —OH, —OCH₃, and —NH₂.

28. The compound of any one of claims 1-19, or a pharmaceutically acceptable salt, solvate, or stereoisomer thereof, wherein:
L¹-R⁷ is

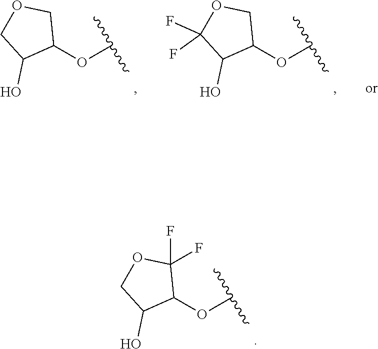 [SMILES: *OC1COCC1O ; *OC1COC(F)(F)C1O ; *OC1C(O)COC1(F)F]

29. The compound of any one of claims 1-19, or a pharmaceutically acceptable salt, solvate, or stereoisomer thereof, wherein:
L¹-R⁷ is

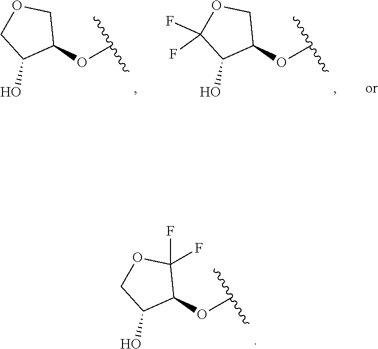 [SMILES: *O[C@@H]1COC[C@H]1O ; *O[C@@H]1COC(F)(F)[C@H]1O ; *O[C@H]1[C@H](O)COC1(F)F]

30. The compound of any one of claims 1-7, or a pharmaceutically acceptable salt, solvate, or stereoisomer thereof, wherein:
R¹ is —CH₃;
R^{2a} and R^{2b} are each hydrogen;
R³ is hydrogen, —(C₁-C₄ alkylene)-OH, or —(C₁-C₄ alkylene)-NH₂;
R⁴ is hydrogen;
each R⁵ and R⁶ is halogen;
L¹ is —X¹— or —X²—(C₁-C₄ alkylene)-, wherein:
X¹ and X² are each selected from: —O—; and
R⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 R¹⁰ groups, wherein:
each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein:
each R⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃.

31. The compound of claim 30, or a pharmaceutically acceptable salt, solvate, or stereoisomer thereof, wherein:
R³ is hydrogen or —(C₁-C₄ alkylene)-OH;
L¹ is —X¹— or —X²—(C₁-C₄ alkylene)-, wherein:
X¹ and X² are each selected from: —O—;
R⁷ is 4- to 8-membered heterocycloalkyl, where the 4- to 8-membered heterocycloalkyl is unsubstituted or substituted by 1, 2, or 3 R¹⁰ groups, wherein:
each R¹⁰ is independently selected from: halogen, —OH, —OMe, —N(R⁸)², —NHSO₂R⁸, and —CH₂CN; and
each R⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl.

32. The compound of claim 30, or a pharmaceutically acceptable salt, solvate, or stereoisomer thereof, wherein:
R³ is CH₂OH;
L is —O—;
R⁷ is 4- to 6-membered heterocycloalkyl, where the 4- to 6-membered heterocycloalkyl is unsubstituted or substituted by 1, 2, or 3 R¹⁰ groups, wherein:
each R¹⁰ is independently selected from: —F, —OH, —OCH₃, and —NH₂.

33. The compound of claim 1, or a pharmaceutically acceptable salt, solvate, or stereoisomer thereof, having the structure of Formula (IV): 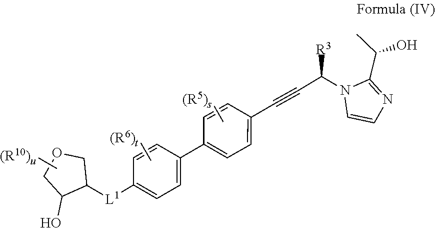
or a pharmaceutically acceptable salt, solvate, or stereoisomer thereof, wherein:
R³ is —(C₁-C₄ alkylene)-OH, —(C₁-C₄ alkylene)-NH₂, —(C₃-C₆ cycloalkylene)-OH, or —(C₃-C₆ cycloalkylene)-NH₂;
each R⁵ and R⁶ is independently —F, —Cl, or C₁-C₄ alkyl;
L¹ is a —(C₁-C₆ alkylene)-, —X¹—, or —X²—(C₁-C₆ alkylene)-, wherein:
X¹ and X² are each selected from: —O—, —N(R⁹)—, —S—, —S(═O)—, —S(═O)₂—, and —S(═O)(═NR⁹)—, wherein:
R⁹ is hydrogen or C₁-C₆ alkyl;
each R¹⁰ is independently selected from: halogen, —OR⁸, —N(R⁸)₂, —CO₂R⁸, —CON(R⁸)₂, —CH₂N(R⁸)₂, —NHCOR⁸, —NHSO₂R⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein:
each R⁸ is independently hydrogen or C₁-C₄ alkyl
s is 0, 1, or 2;
t is 0, 1, or 2; and
u is 0, 1, or 2.

34. The compound of claim 33, or a pharmaceutically acceptable salt, solvate, or stereoisomer thereof, having the structure of Formula (V): 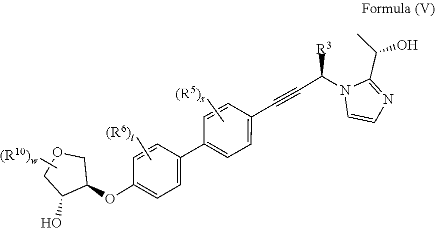
or a pharmaceutically acceptable salt, solvate, or stereoisomer thereof.

35. The compound of claim 33 or claim 34, or a pharmaceutically acceptable salt, solvate, or stereoisomer thereof, wherein s is 1, or 2; and at least one R⁵ is —F.

36. The compound of any one of claims 33-35, or a pharmaceutically acceptable salt, solvate, or stereoisomer thereof, wherein t is 1, or 2; and at least one R⁶ is —F.

37. The compound of any one of claims 33-36, or a pharmaceutically acceptable salt, solvate, or stereoisomer thereof, wherein u is 1, or 2; and at least one R¹⁰ is —F.

38. The compound of any one of claims 33-37, or a pharmaceutically acceptable salt, solvate, or stereoisomer thereof, wherein R³ is —(C₁-C₄ alkylene)-OH.

39. The compound of any one of claims 33-37, or a pharmaceutically acceptable salt, solvate, or stereoisomer thereof, wherein R³ is —CH₂OH.

40. The compound of claim 1, selected from: 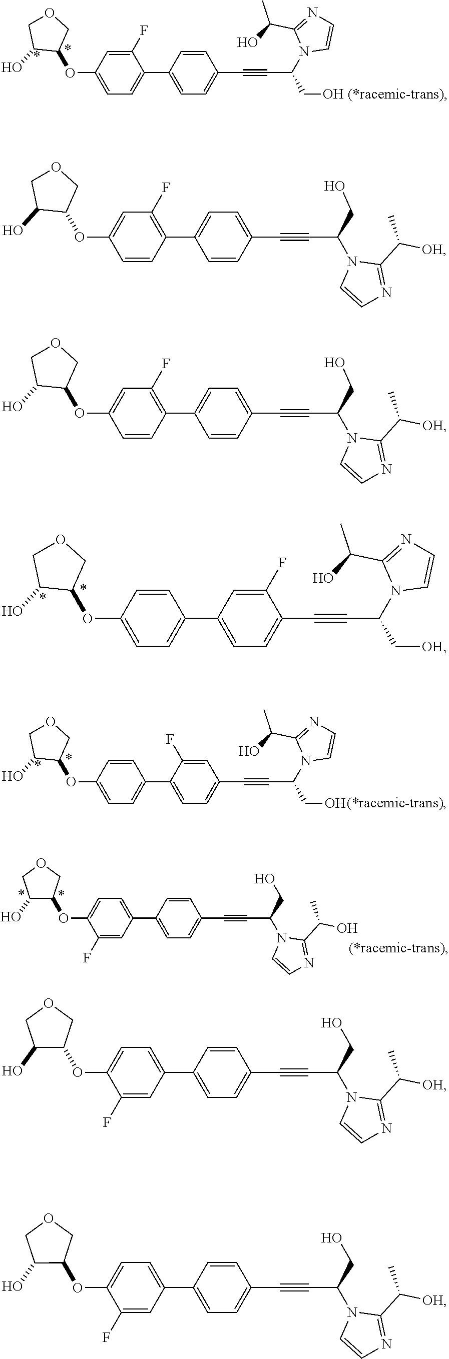 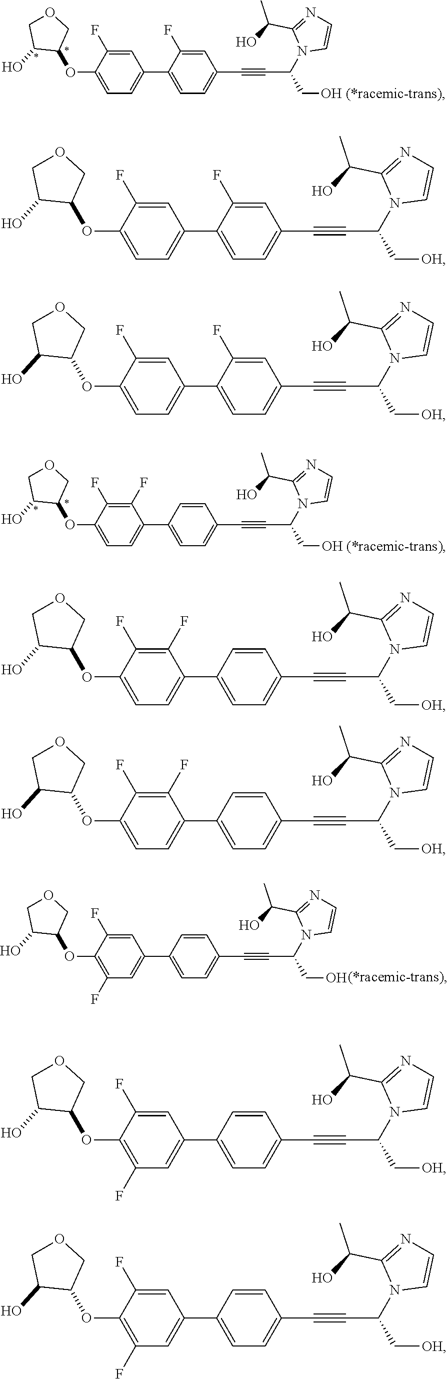 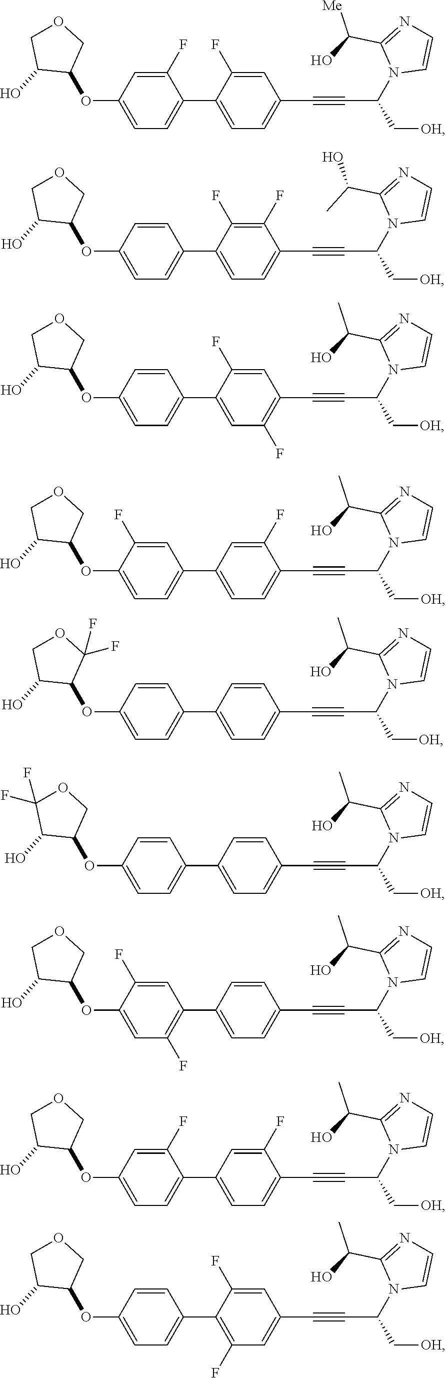 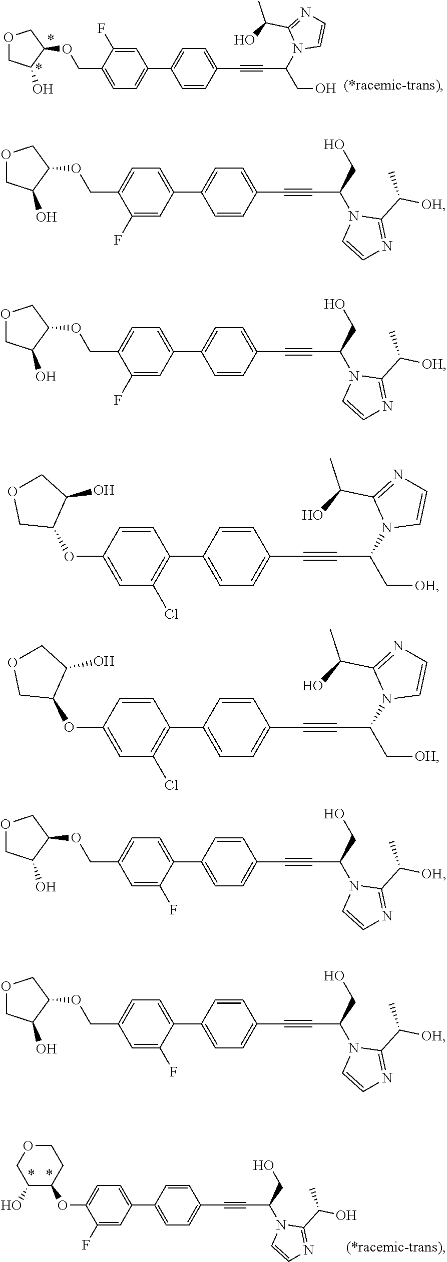 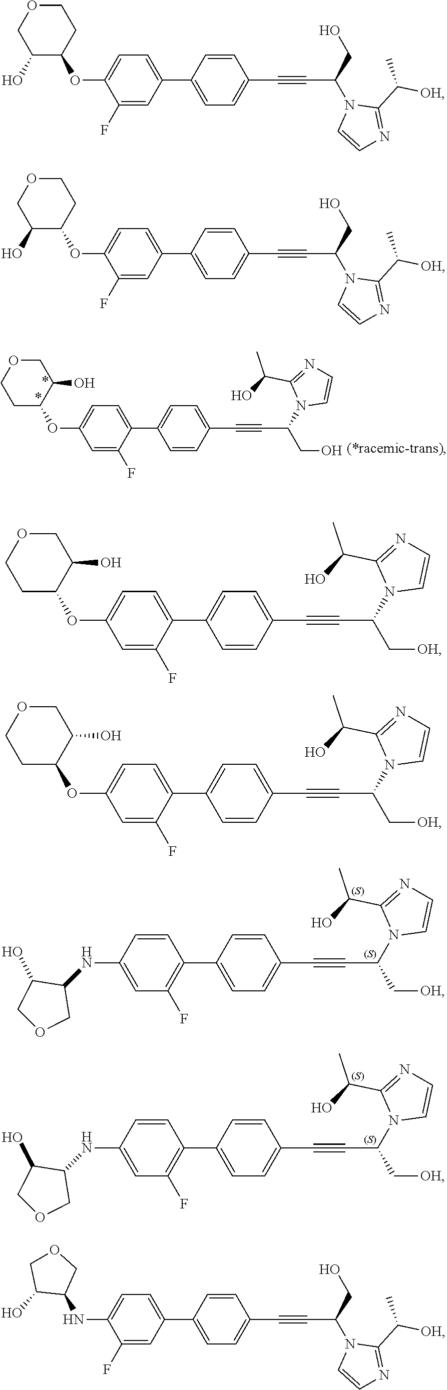 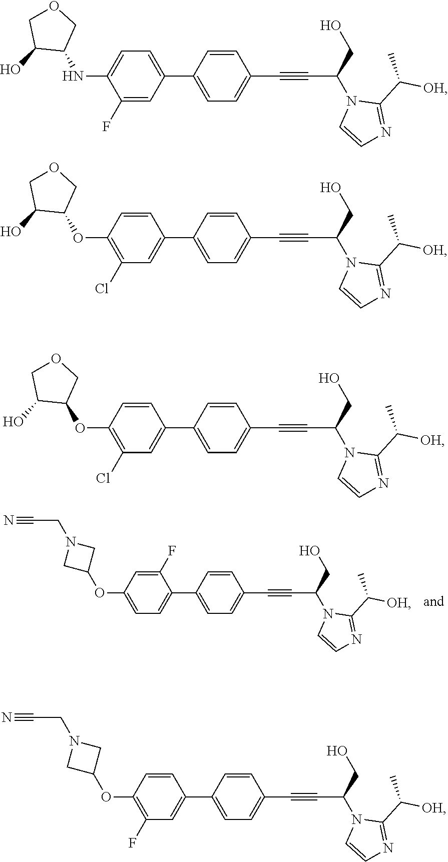 [SMILES: C[C@H](O)c1nccn1[C@@H](C#Cc1ccc(-c2ccc(N[C@H]3COC[C@@H]3O)c(F)c2)cc1)CO ; C[C@H](O)c1nccn1[C@@H](C#Cc1ccc(-c2ccc(O[C@H]3COC[C@@H]3O)c(Cl)c2)cc1)CO ; C[C@H](O)c1nccn1[C@@H](C#Cc1ccc(-c2ccc(O[C@@H]3COC[C@H]3O)c(Cl)c2)cc1)CO ; C[C@H](O)c1nccn1[C@@H](C#Cc1ccc(-c2ccc(OC3CN(CC#N)C3)cc2F)cc1)CO ; C[C@H](O)c1nccn1[C@@H](C#Cc1ccc(-c2ccc(OC3CN(CC#N)C3)c(F)c2)cc1)CO]
or a pharmaceutically acceptable salt, solvate, or stereoisomer thereof.

41. The compound of claim 1, selected from: 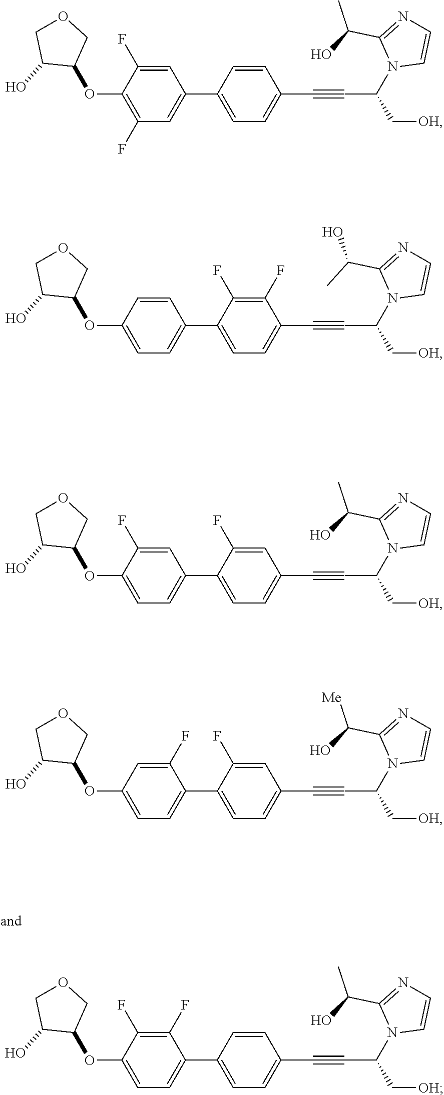 [SMILES: C[C@H](O)c1nccn1[C@@H](C#Cc1ccc(-c2cc(F)c(O[C@@H]3COC[C@H]3O)c(F)c2)cc1)CO ; C[C@H](O)c1nccn1[C@@H](C#Cc1ccc(-c2ccc(O[C@@H]3COC[C@H]3O)cc2)c(F)c1F)CO ; C[C@H](O)c1nccn1[C@@H](C#Cc1ccc(-c2ccc(O[C@@H]3COC[C@H]3O)c(F)c2)c(F)c1)CO ; C[C@H](O)c1nccn1[C@@H](C#Cc1ccc(-c2ccc(O[C@@H]3COC[C@H]3O)cc2F)c(F)c1)CO ; C[C@H](O)c1nccn1[C@@H](C#Cc1ccc(-c2ccc(O[C@@H]3COC[C@H]3O)c(F)c2F)cc1)CO]
or a pharmaceutically acceptable salt, solvate, or stereoisomer thereof.

42. A compound of Formula (VI): 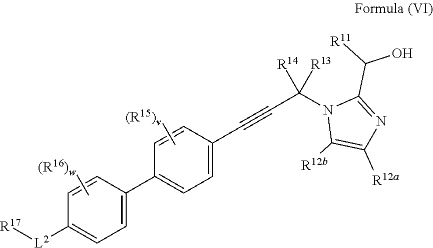
or a pharmaceutically acceptable salt, solvate, or stereoisomer thereof, wherein:
R¹¹ is C₁-C₄ alkyl;
R^{12a} and R^{12b} are each independently hydrogen, halogen, or C₁-C₄ alkyl;
R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂;
R¹⁴ is hydrogen or C₁-C₄ alkyl;
each R¹⁵ and R¹⁶ is independently halogen, or C₁-C₄ alkyl;
R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 groups independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; wherein:
each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃;
or two R¹⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo;
L² is a bond, —(C₁-C₆ alkylene)-, —X³—, or —X⁴—(C₁-C₆ alkylene)-, wherein:
X³ and X⁴ are each independently selected from: —O—, —N(R¹⁹)—, —S—, —S(═O)—, —S(═O)₂—, and —S(═O)(═NR¹⁹)—, wherein:
R¹⁹ is hydrogen or C₁-C₆ alkyl;
provided that when L² is a bond, —(C₁-C₆ alkylene)-, —O—, or —O—(C₁-C₆ alkylene)- then at least one of the following:
(i) R¹³ is —(C₃-C₆ cycloalkylene)-OH or —(C₃-C₆ cycloalkylene)-NH₂;
(ii) R¹⁴ is C₁-C₄ alkyl,
(iii) R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂; or
(iv) L² is —O—(C₁-C₆ alkylene)- and R¹⁷ is an unsubstituted or substituted 5-membered heterocycloalkyl;
v is 0, 1, or 2; and
w is 0, 1, or 2.

43. The compound of claim 42, or a pharmaceutically acceptable salt, solvate, or stereoisomer thereof, wherein:
R¹¹ is —CH₃.

44. The compound of claim 42 or claim 43, or a pharmaceutically acceptable salt, solvate, or stereoisomer thereof, wherein:
R^{12a} is hydrogen; and
R^{12b} is hydrogen.

45. The compound of any one of claims 42-44, wherein:
R¹⁴ is hydrogen.

46. The compound of any one of claims 42-45, or a pharmaceutically acceptable salt, solvate, or stereoisomer thereof, wherein:
each R¹⁵ is independently —F, —Cl, or —CH₃.

47. The compound of any one of claims 42-45, or a pharmaceutically acceptable salt, solvate, or stereoisomer thereof, wherein:
each R¹⁵ is independently —F.

48. The compound of any one of claims 42-47, or a pharmaceutically acceptable salt, solvate, or stereoisomer thereof, wherein:
each R¹⁶ is independently —F, —Cl, or —CH₃.

49. The compound of any one of claims 42-47, or a pharmaceutically acceptable salt, solvate, or stereoisomer thereof, wherein:
each R¹⁶ is independently —F.

50. The compound of claim any one of claims 42-49, or a pharmaceutically acceptable salt, solvate, or stereoisomer thereof, having the structure of Formula (VIIa): 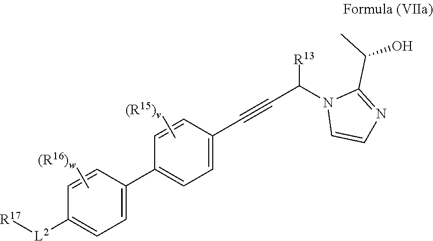
or a pharmaceutically acceptable salt, solvate, or stereoisomer thereof.

51. The compound of claim any one of claims 42-49, or a pharmaceutically acceptable salt, solvate, or stereoisomer thereof, having the structure of Formula (VIIIa): 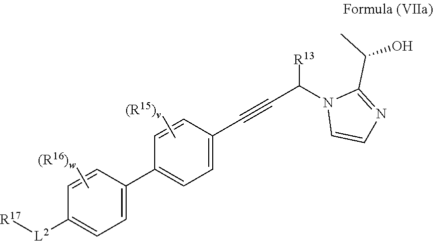
or a pharmaceutically acceptable salt, solvate, or stereoisomer thereof.

52. The compound of claim any one of claims 42-50, or a pharmaceutically acceptable salt, solvate, or stereoisomer thereof, wherein:
v is 1 or 2.

53. The compound of claim any one of claims 42-50, or a pharmaceutically acceptable salt, solvate, or stereoisomer thereof, wherein:
v is 0.

54. The compound of claim any one of claims 42-53, or a pharmaceutically acceptable salt, solvate, or stereoisomer thereof, wherein
w is 1 or 2.

55. The compound of claim any one of claims 42-53, or a pharmaceutically acceptable salt, solvate, or stereoisomer thereof, wherein:
w is 0.

56. The compound of claim any one of claims 42-55, or a pharmaceutically acceptable salt, solvate, or stereoisomer thereof, wherein
L² is —X³— or —X⁴—(C₁-C₆ alkylene)-; and
X³ and X⁴ are each independently selected from: —N(R¹⁹)—, —S—, —S(═O)—, —S(═O)₂—, and —S(═O)(═NR¹⁹).

57. The compound of claim 56, or a pharmaceutically acceptable salt, solvate, or stereoisomer thereof, wherein:
R³ is hydrogen or —(C₁-C₄ alkylene)-OH.

58. The compound of claim 56, or a pharmaceutically acceptable salt, solvate, or stereoisomer thereof, wherein:
R³ is —(C₁-C₄ alkylene)-OH.

59. The compound of claim 56, or a pharmaceutically acceptable salt, solvate, or stereoisomer thereof, wherein:
R³ is —CH₂OH.

60. The compound of any one of claims 56-59, or a pharmaceutically acceptable salt, solvate, or stereoisomer thereof, wherein:
R¹⁷ is C₁-C₆ alkyl, C₁-C₆ heteroalkyl, C₃-C₆ cycloalkyl, or 4- to 8-membered heterocycloalkyl; wherein the alkyl, heteroalkyl, cycloalkyl, or heterocycloalkyl is unsubstituted or substituted by 1, 2, or 3 groups independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein:
each R¹⁸ is independently hydrogen, C₁-C₄ alkyl, —C(═O)—C₁-C₄ alkyl, or 4- to 6-membered heterocycloalkyl, wherein the alkyl or heterocycloalkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, —C(═NH)NH₂, oxo, phenyl, and monocyclic heteroaryl which is unsubstituted or substituted by 1 or 2 groups selected from —F, —CN, —OH, —NH₂, —OMe, —N(CH₃)₂, —CO₂H, —CONH₂, and —SO₂CH₃.

61. The compound of any one of claims 56-59, or a pharmaceutically acceptable salt, solvate, or stereoisomer thereof, wherein:
R¹⁷ is 4- to 8-membered heterocycloalkyl, where the 4- to 8-membered heterocycloalkyl is unsubstituted or substituted by 1, 2, or 3 groups independently selected from: halogen, —OR¹⁸, —N(R¹⁸)₂, —NHSO₂R¹⁸, —CH₂CN, C₁-C₄ alkyl, —C(═O)—C₁-C₄ hydroxyalkyl, C₁-C₄ hydroxyalkyl, and C₁-C₄ methoxyalkyl; and
each R¹⁸ is independently hydrogen, C₁-C₄ alkyl, —C(═O)—C₁-C₄ alkyl, or 4- to 6-membered heterocycloalkyl, wherein the alkyl is unsubstituted or substituted by 1, 2, or 3 groups independently selected from —F, —CN, —OH, —CO₂H, —C(═NH)NH₂, and 5-membered monocyclic heteroaryl which is unsubstituted or substituted by 1 —CONH₂ group.

62. The compound of any one of claims 56-59, or a pharmaceutically acceptable salt, solvate, or stereoisomer thereof, wherein:
R¹⁷ is 4- to 8-membered heterocycloalkyl, where the 4- to 8-membered heterocycloalkyl is unsubstituted or substituted by 1, 2, or 3 groups independently selected from: halogen, —OH, —OMe, —N(R⁸)₂, —NHSO₂R¹⁸, and —CH₂CN; and
each R¹⁸ is independently hydrogen, C₁-C₂ alkyl, or —C(═O)—C₁-C₂ alkyl, wherein the alkyl is unsubstituted or substituted by 1 or 2 groups independently selected from —CN, —OH, and oxadiazolyl.

63. The compound of any one of claims 56-59, or a pharmaceutically acceptable salt, solvate, or stereoisomer thereof, wherein:
R¹⁷ is 4- to 6-membered heterocycloalkyl, where the 4- to 6-membered heterocycloalkyl is unsubstituted or substituted by 1, 2, or 3 groups independently selected from:
—F, —OH, —OCH₃, and —NH₂.

64. The compound of claim 42, or a pharmaceutically acceptable salt, solvate, or stereoisomer thereof, having the structure of Formula (IX): 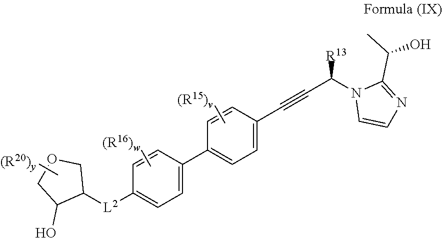
or a pharmaceutically acceptable salt, solvate, or stereoisomer thereof, wherein:
R¹³ is —(C₁-C₄ alkylene)-OH, —(C₁-C₄ alkylene)-NH₂, —(C₃-C₆ cycloalkylene)-OH, or —(C₃-C₆ cycloalkylene)-NH₂;
each R¹⁵ and R¹⁶ is independently —F, —Cl, or C₁-C₄ alkyl;
L² is —X³— or —X⁴—(C₁-C₆ alkylene)-; and
X³ and X⁴ are each independently selected from: —N(R¹⁹)—, —S—, —S(═O)—, —S(═O)₂—, and —S(═O)(═NR¹⁹).
R¹⁹ is hydrogen or C₁-C₆ alkyl;
each R²⁰ is independently selected from: halogen, —OR¹⁸, —N(R¹⁸)₂, —CO₂R¹⁸, —CON(R¹⁸)₂, —CH₂N(R¹⁸)₂, —NHCOR¹⁸, —NHSO₂R¹⁸, —CH₂CN, C₁-C₄ alkyl, C₁-C₄ hydroxyalkyl, —C(═O)—C₁-C₄ hydroxyalkyl, C₁-C₄ methoxyalkyl, C₁-C₄ aminoalkyl, and oxo; further wherein:
each R¹⁸ is independently hydrogen or C₁-C₄ alkyl
v is 0, 1, or 2;
w is 0, 1, or 2; and
y is 0, 1, or 2.

65. The compound of claim 64, or a pharmaceutically acceptable salt, solvate, or stereoisomer thereof, having the structure of Formula (X): 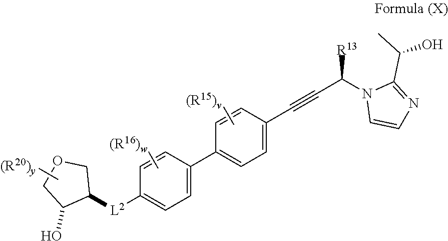
or a pharmaceutically acceptable salt, solvate, or stereoisomer thereof.

66. The compound of claim 64 or claim 65, or a pharmaceutically acceptable salt, solvate, or stereoisomer thereof, wherein:
R¹³ is —(C₁-C₄ alkylene)-OH.

67. The compound of any one of claims 64 64-66, or a pharmaceutically acceptable salt, solvate, or stereoisomer thereof, wherein:
R¹³ is —CH₂OH.

68. The compound of any one of claims 64-67, or a pharmaceutically acceptable salt, solvate, or stereoisomer thereof, wherein:
w and v are each 0.

69. The compound of any one of claims 42-55, or a pharmaceutically acceptable salt, solvate, or stereoisomer thereof, wherein:
L² is a bond, —(C₁-C₆ alkylene)-, —O—, or —O—(C₁-C₆ alkylene)-; and
(i) R¹³ is —(C₃-C₆ cycloalkylene)-OH or —(C₃-C₆ cycloalkylene)-NH₂;
(ii) R¹⁴ is C₁-C₄ alkyl,
(iii) R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂; or
(iv) L² is —O—(C₁-C₆ alkylene)- and R¹⁷ is an unsubstituted or substituted 5-membered heterocycloalkyl.

70. The compound of claim 69, or a pharmaceutically acceptable salt, solvate, or stereoisomer thereof, wherein:
R¹³ is —(C₃-C₆ cycloalkylene)-OH or —(C₃-C₆ cycloalkylene)-NH₂.

71. The compound of claim 70, or a pharmaceutically acceptable salt, solvate, or stereoisomer thereof, wherein:
R¹³ is -(cyclopropylene)-OH or -(cyclopropylene)-NH₂.

72. The compound of claim 70, or a pharmaceutically acceptable salt, solvate, or stereoisomer thereof, wherein:
R¹³ is

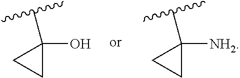 [SMILES: *C1(O)CC1 ; "or" ; *C1(N)CC1]

73. The compound of claim 69, or a pharmaceutically acceptable salt, solvate, or stereoisomer thereof, wherein:
R¹⁴ is C₁-C₄ alkyl.

74. The compound of claim 73, or a pharmaceutically acceptable salt, solvate, or stereoisomer thereof, wherein:
R¹⁴ is methyl.

75. The compound of claim 69, or a pharmaceutically acceptable salt, solvate, or stereoisomer thereof, wherein:
R¹⁷ is an unsubstituted or substituted 7- to 8-membered heterocycloalkyl, an unsubstituted or substituted sulfur-containing heterocycloalkyl, an unsubstituted or substituted a bicyclic heterocycloalkyl, an unsubstituted or substituted a 6-membered oxygen-containing heterocycloalkyl, a disubstituted or trisubstituted cycloalkyl, or a 4-membered heterocycloalkyl substituted by at least one —N(R¹⁸)₂.

76. The compound of claim 75, or a pharmaceutically acceptable salt, solvate, or stereoisomer thereof, wherein:
R¹⁷ is an unsubstituted or substituted hexahydrofuro[3,2-b]furan, an unsubstituted or substituted tetrahydrothiophene-1-oxide, an unsubstituted or substituted 3-oxabicyclo[3.1.0]hexane, an unsubstituted or substituted tetrahydropyran, a disubstituted or trisubstituted cyclopropyl, or an oxetane substituted by at least one —N(R¹⁸).

77. The compound of claim 76, or a pharmaceutically acceptable salt, solvate, or stereoisomer thereof, wherein:
R¹⁷ is

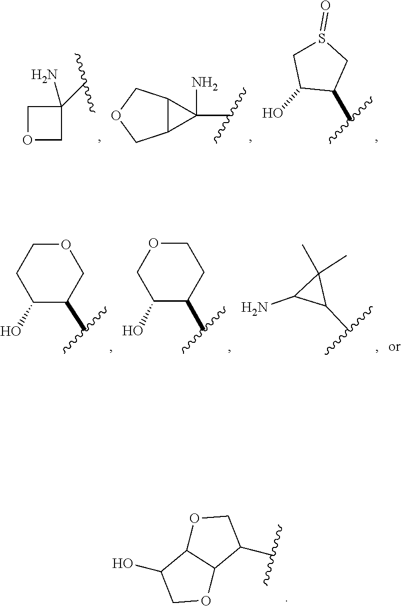

78. The compound of any one of claims 75-77, or a pharmaceutically acceptable salt, solvate, or stereoisomer thereof, wherein:
L² is a bond, —CH₂—, —O—, or —O—CH₂—.

79. The compound of claim 78, or a pharmaceutically acceptable salt, solvate, or stereoisomer thereof, wherein:
-L²-R¹⁷ is

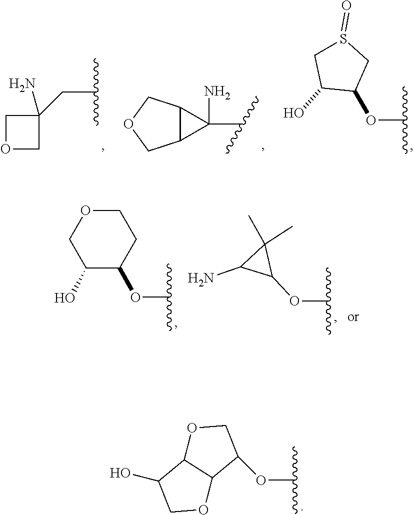

80. The compound of claim 69, or a pharmaceutically acceptable salt, solvate, or stereoisomer thereof, wherein:
L² is —O—(C₁-C₆ alkylene)-; and
R¹⁷ is an unsubstituted or substituted 5-membered heterocycloalkyl.

81. The compound of claim 80, or a pharmaceutically acceptable salt, solvate, or stereoisomer thereof, wherein:
R¹⁷ is an unsubstituted or substituted tetrahydrofuranyl.

82. The compound of claim 81, or a pharmaceutically acceptable salt, solvate, or stereoisomer thereof, wherein:
R¹⁷ is

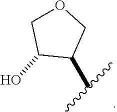 [SMILES: *[C@@H]1COC[C@H]1O]

83. The compound of any one of claims 80-82, or a pharmaceutically acceptable salt, solvate, or stereoisomer thereof, wherein:
L² is —O—CH₂—.

84. The compound of claim 83, or a pharmaceutically acceptable salt, solvate, or stereoisomer thereof, wherein:
L²-R¹⁷ is

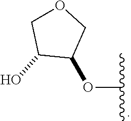 [SMILES: *O[C@@H]1COC[C@H]1O]

85. The compound of claim 42, selected from: 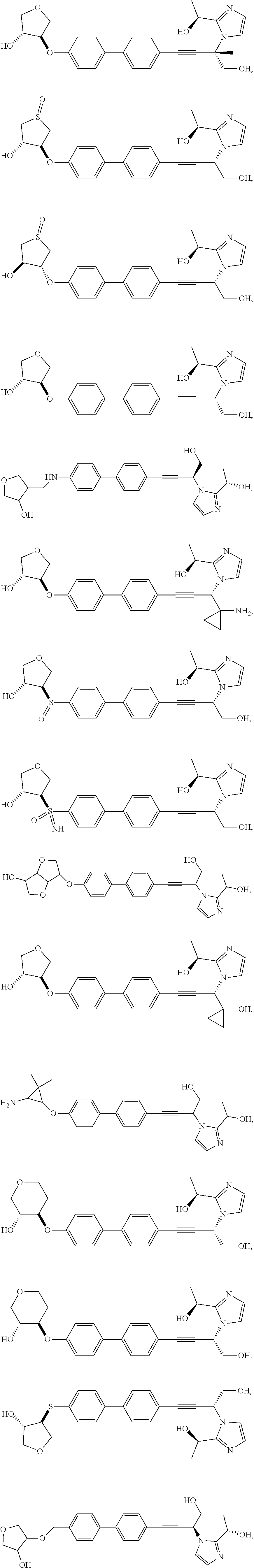 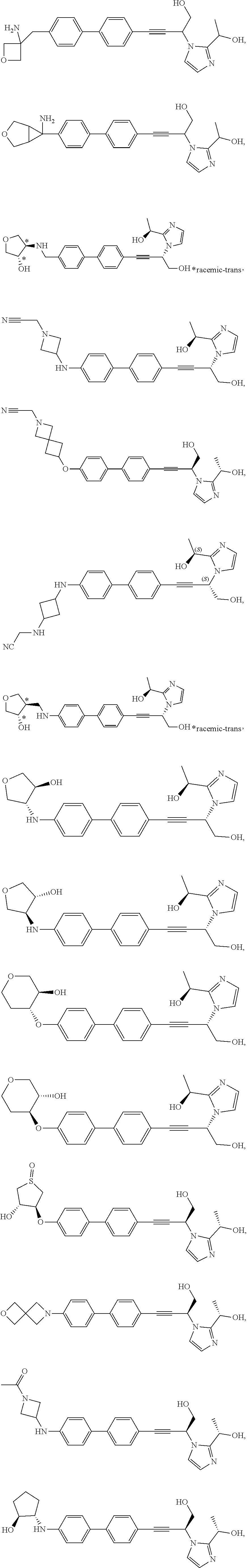 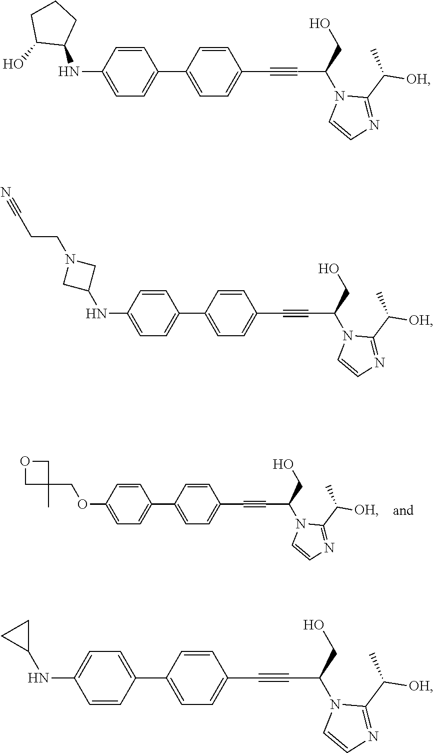
or a pharmaceutically acceptable salt, solvate, or stereoisomer thereof.

86. The compound of claim 42, selected from: 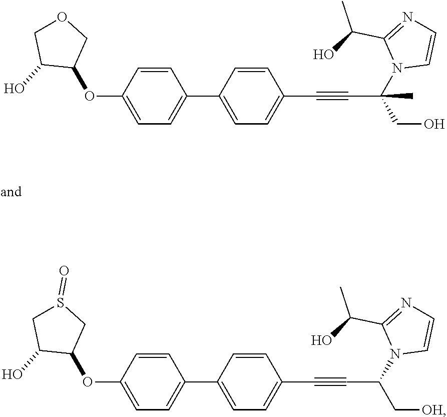 [SMILES: C[C@H](O)c1nccn1[C@@](C)(C#Cc1ccc(-c2ccc(O[C@@H]3COC[C@H]3O)cc2)cc1)CO ; C[C@H](O)c1nccn1[C@@H](C#Cc1ccc(-c2ccc(O[C@@H]3CS(=O)C[C@H]3O)cc2)cc1)CO]
or a pharmaceutically acceptable salt, solvate, or stereoisomer thereof.

87. A compound of Formula (XI): 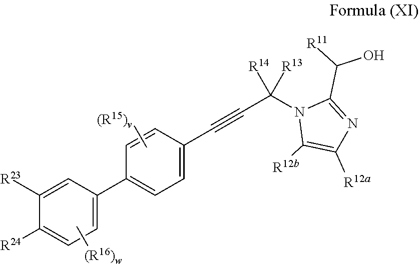
or a pharmaceutically acceptable salt, solvate, or stereoisomer thereof, wherein:
R¹¹ is C₁-C₄ alkyl;
R^{12a} and R^{12b} are each independently hydrogen, halogen, or C₁-C₄ alkyl;
R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂;
R¹⁴ is hydrogen or C₁-C₄ alkyl;
each R¹⁵ and R¹⁶ is independently halogen, or C₁-C₄ alkyl;
R²³ and R²⁴ are taken together with the intervening carbon atoms connecting R²³ to R²⁴ to form a Ring A that is C₃-C₆ cycloalkyl or 4- to 8-membered heterocycloalkyl, wherein the C₃-C₆ cycloalkyl or 4- to 8-membered heterocycloalkyl is unsubstituted or substituted by 1, 2, or 3 groups independently selected from: halogen, —OR²⁵, —N(R²⁵)₂, —CO₂R²⁵, —COR²⁵, —CON(R²⁵)₂, C₁-C₄ alkyl, C₁-C₄ hydroxyalkyl, C₁-C₄ aminoalkyl, and oxo; wherein:
each R²⁵ is independently hydrogen or C₁-C₄ alkyl, wherein the alkyl is unsubstituted or substituted by 1, 2, 3, or 4 groups independently selected from —F, —CN, —OH, —CH₂OH, —NH₂, —OMe, —N(CH₃)₂, —CO₂H, —CONH₂, —SO₂CH₃, and oxo;
or two R²⁵ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo;
v is 0, 1, or 2; and
w is 0, 1, or 2.

88. The compound of claim 87, or a pharmaceutically acceptable salt, solvate, or stereoisomer thereof, wherein:
R¹³ is —(C₁-C₄ alkylene)-OH.

89. The compound of any one of claims 87-88, or a pharmaceutically acceptable salt, solvate, or stereoisomer thereof, wherein:
R¹³ is —CH₂OH.

90. The compound of any one of claims 87-89, or a pharmaceutically acceptable salt, solvate, or stereoisomer thereof, wherein:
R¹⁴ is hydrogen.

91. The compound of any one of claims 87-90, or a pharmaceutically acceptable salt, solvate, or stereoisomer thereof, wherein:
R^{12a} and R^{12b} is hydrogen.

92. The compound of any one of claims 87-91, or a pharmaceutically acceptable salt, solvate, or stereoisomer thereof, wherein:
R¹¹ is —CH₃.

93. The compound of any one of claims 87-92, or a pharmaceutically acceptable salt, solvate, or stereoisomer thereof, wherein:
w is 0 and v is 0.

94. The compound of any one of claims 87-93, or a pharmaceutically acceptable salt, solvate, or stereoisomer thereof, wherein:
R²³ and R²⁴ are taken together with the intervening carbon atoms connecting R²³ to R²⁴ to form a Ring A that is 4- to 8-membered heterocycloalkyl, wherein the 4- to 8-membered heterocycloalkyl is unsubstituted or substituted by 1, 2, or 3 groups independently selected from: halogen, —OR²⁵, —N(R²⁵)₂, —CO₂R²⁵, —COR²⁵, —CON(R²⁵)₂, C₁-C₄ alkyl, C₁-C₄ hydroxyalkyl, C₁-C₄ aminoalkyl, and oxo.

95. The compound of any one of claims 87-94, or a pharmaceutically acceptable salt, solvate, or stereoisomer thereof, wherein:
R²³ and R²⁴ are taken together with the intervening carbon atoms connecting R²³ to R²⁴ to form a Ring A that is 5- to 6-membered heterocycloalkyl, wherein the 5- to 6-membered heterocycloalkyl is unsubstituted or substituted by 1, 2, or 3 groups independently selected from: halogen, C₁-C₄ alkyl, C₁-C₄ hydroxyalkyl, and oxo.

96. The compound of any one of claims 87-95, or a pharmaceutically acceptable salt, solvate, or stereoisomer thereof, wherein:
R²³ and R²⁴ are taken together with the intervening carbon atoms connecting R²³ to R²⁴ to form a Ring A that is

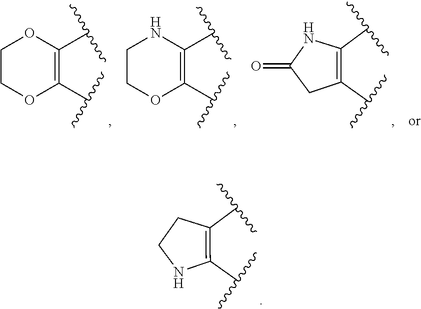

97. The compound of claim 87, selected from: 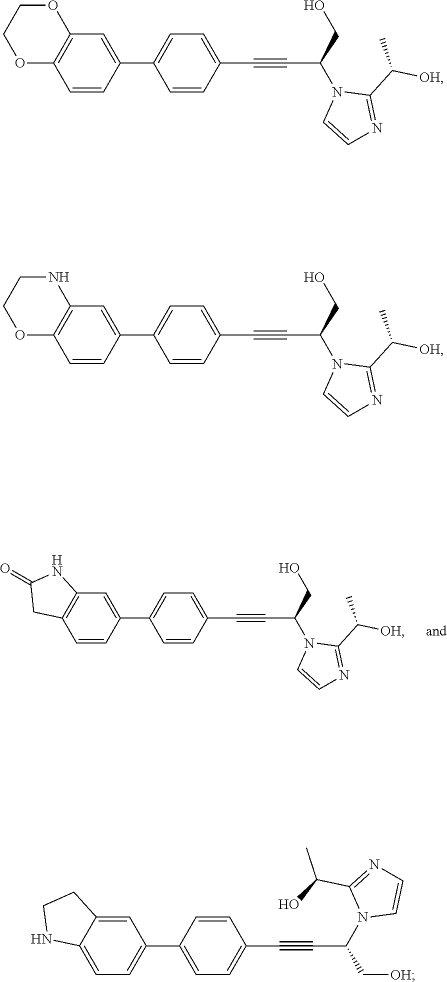 [SMILES: C[C@H](O)c1nccn1[C@@H](C#Cc1ccc(-c2ccc3c(c2)OCCO3)cc1)CO ; C[C@H](O)c1nccn1[C@@H](C#Cc1ccc(-c2ccc3c(c2)NCCO3)cc1)CO ; C[C@H](O)c1nccn1[C@@H](C#Cc1ccc(-c2ccc3c(c2)NC(=O)C3)cc1)CO ; C[C@H](O)c1nccn1[C@@H](C#Cc1ccc(-c2ccc3c(c2)CCN3)cc1)CO]
or a pharmaceutically acceptable salt, solvate, or stereoisomer thereof.

98. A compound of Formula (XII): 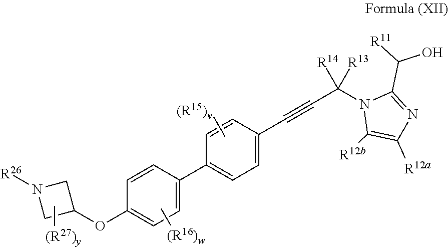
or a pharmaceutically acceptable salt, solvate, or stereoisomer thereof, wherein:
R¹¹ is C₁-C₄ alkyl;
R^{12a} and R^{12b} are each independently hydrogen, halogen, or C₁-C₄ alkyl;
R¹³ is hydrogen, —(C₁-C₄ alkylene)-OH, —(C₁-C₄ alkylene)-NH₂, —(C₃-C₆ cycloalkylene)-OH, or —(C₃-C₆ cycloalkylene)-NH₂;
R¹⁴ is hydrogen or C₁-C₄ alkyl;
each R¹⁵ and R¹⁶ is independently halogen, or C₁-C₄ alkyl;
R²⁶ is C₁-C₆alkyl, C₁-C₆ fluoroalkyl, 4- to 6-membered heterocycloalkyl, heteroaryl, —C(═O)—C₁-C₆ alkyl, —C(═O)—N(R²⁸)₂, —CH₂—C(═O)—N(R²⁸)₂, or —S(O)₂—C₁-C₆ alkyl, wherein C₁-C₆ fluoroalkyl, C₁-C₆ alkyl, 4- to 6-membered heterocycloalkyl, and heteroaryl is unsubstituted or substituted with 1, 2, or 3 groups independently selected from halogen, C₁-C₆ alkyl, —OH, —NH₂, and —CN;
each R²⁷ is independently hydrogen or C₁-C₆ alkyl;
each R²⁸ is independently hydrogen or C₁-C₆ alkyl;
or two R²⁸ attached to the same nitrogen are taken together to form a 4- to 6-membered heterocycloalkyl which is unsubstituted or substituted by 1, 2, or 3 groups independently selected from —F, —CN, —OH, —NH₂, —OMe, —CO₂H, —CONH₂, —SO₂CH₃, and oxo;
y is 0, 1, or 2;
v is 0, 1, or 2; and
w is 0, 1, or 2.

99. The compound of claim 98, or a pharmaceutically acceptable salt, solvate, or stereoisomer thereof, wherein:
R¹³ is —(C₁-C₄ alkylene)-OH.

100. The compound of any one of claims 98-99, or a pharmaceutically acceptable salt, solvate, or stereoisomer thereof, wherein:
R¹³ is —CH₂OH.

101. The compound of any one of claims 98-100, or a pharmaceutically acceptable salt, solvate, or stereoisomer thereof, wherein:
R¹⁴ is hydrogen.

102. The compound of any one of claims 98-101, or a pharmaceutically acceptable salt, solvate, or stereoisomer thereof, wherein:
R^{12a} and R^{12b} is hydrogen.

103. The compound of any one of claims 98-102, or a pharmaceutically acceptable salt, solvate, or stereoisomer thereof, wherein:
R¹¹ is —CH₃.

104. The compound of any one of claims 98-103, or a pharmaceutically acceptable salt, solvate, or stereoisomer thereof, wherein:
w is 0 and v is 0.

105. The compound of any one of claims 98-104, or a pharmaceutically acceptable salt, solvate, or stereoisomer thereof, wherein:
R²⁷ is hydrogen.

106. The compound of any one of claims 98-105, or a pharmaceutically acceptable salt, solvate, or stereoisomer thereof, wherein:
R²⁷ is —CH₃.

107. The compound of any one of claims 98-106, or a pharmaceutically acceptable salt, solvate, or stereoisomer thereof, wherein:
R²⁶ is C₁-C₆alkyl, C₁-C₆fluoroalkyl, oxetanyl, imidazolyl, —C(═O)—CH₃, —S(O)₂—CH₃, —C(═O)—C₁-C₆ alkyl, —C(═O)—N(R²⁸)₂, —CH₂—C(═O)—NH(CH₃), wherein C₁-C₆fluoroalkyl, C₁-C₆ alkyl, oxetanyl, and imidazolyl is unsubstituted or substituted with 1, 2, or 3 groups independently selected from halogen, —CH₃, —OH, —NH₂, and —CN;
each R²⁸ is independently hydrogen or —CH₃;
or two R²⁸ attached to the same nitrogen are taken together to form morpholinyl.

108. The compound of any one of claims 98-107, or a pharmaceutically acceptable salt, solvate, or stereoisomer thereof, wherein:
R²⁶ is

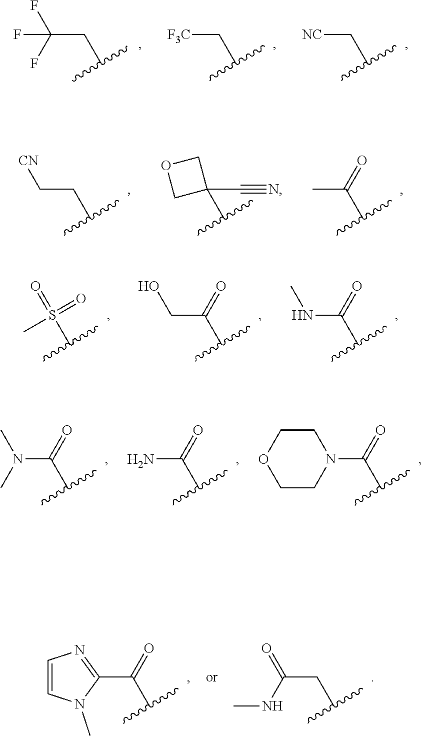

109. The compound of claim 98, selected from: 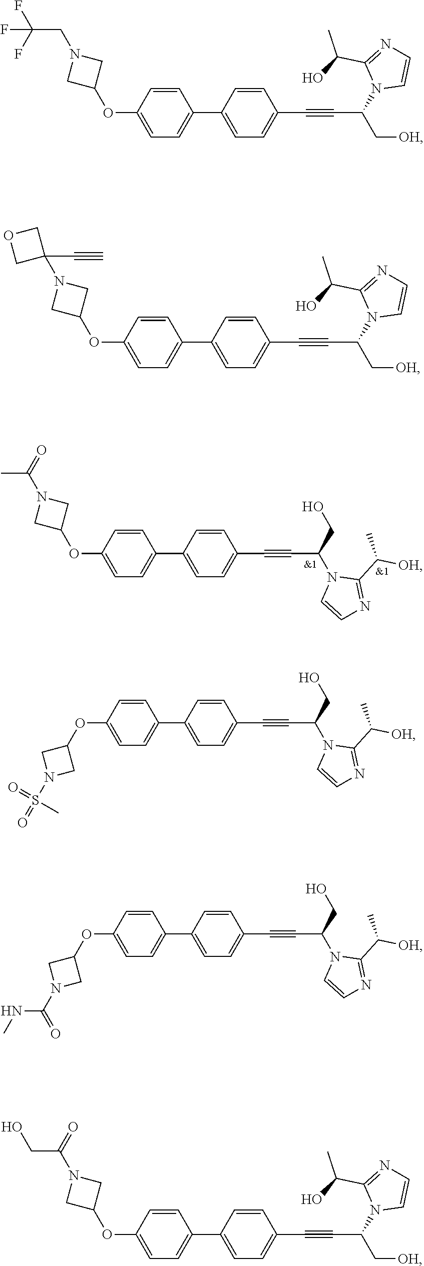 [SMILES: C[C@H](O)c1nccn1[C@@H](C#Cc1ccc(-c2ccc(OC3CN(CC(F)(F)F)C3)cc2)cc1)CO ; C#CC1(N2CC(Oc3ccc(-c4ccc(C#C[C@@H](CO)n5ccnc5[C@H](C)O)cc4)cc3)C2)COC1 ; CC(=O)N1CC(Oc2ccc(-c3ccc(C#C[C@@H](CO)n4ccnc4[C@H](C)O)cc3)cc2)C1 ; C[C@H](O)c1nccn1[C@@H](C#Cc1ccc(-c2ccc(OC3CN(S(C)(=O)=O)C3)cc2)cc1)CO ; CNC(=O)N1CC(Oc2ccc(-c3ccc(C#C[C@@H](CO)n4ccnc4[C@H](C)O)cc3)cc2)C1 ; C[C@H](O)c1nccn1[C@@H](C#Cc1ccc(-c2ccc(OC3CN(C(=O)CO)C3)cc2)cc1)CO] 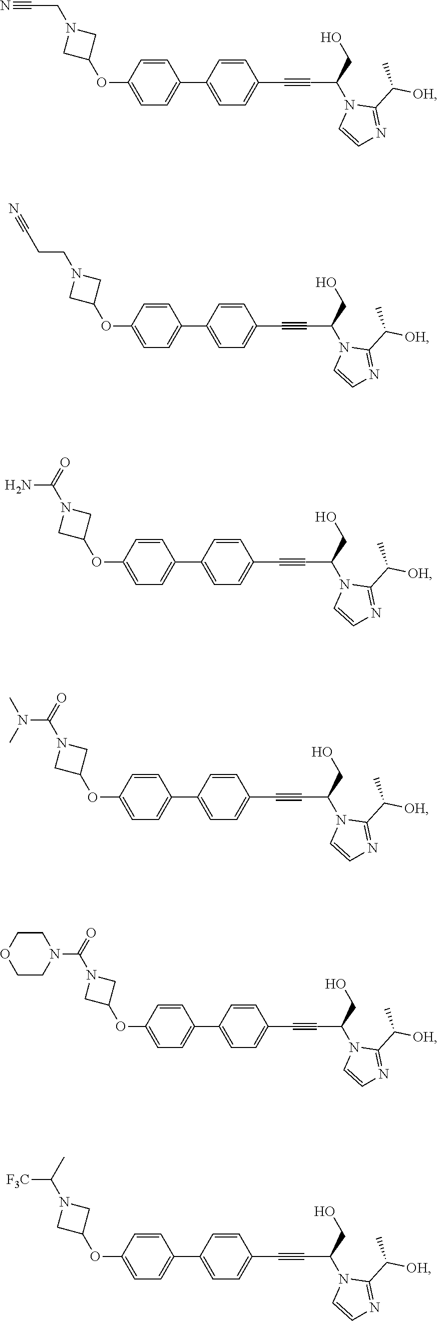 [SMILES: C[C@H](O)c1nccn1[C@@H](C#Cc1ccc(-c2ccc(OC3CN(CC#N)C3)cc2)cc1)CO ; C[C@H](O)c1nccn1[C@@H](C#Cc1ccc(-c2ccc(OC3CN(CCC#N)C3)cc2)cc1)CO ; C[C@H](O)c1nccn1[C@@H](C#Cc1ccc(-c2ccc(OC3CN(C(N)=O)C3)cc2)cc1)CO ; C[C@H](O)c1nccn1[C@@H](C#Cc1ccc(-c2ccc(OC3CN(C(=O)N(C)C)C3)cc2)cc1)CO ; C[C@H](O)c1nccn1[C@@H](C#Cc1ccc(-c2ccc(OC3CN(C(=O)N4CCOCC4)C3)cc2)cc1)CO ; CC(N1CC(Oc2ccc(-c3ccc(C#C[C@@H](CO)n4ccnc4[C@H](C)O)cc3)cc2)C1)C(F)(F)F] 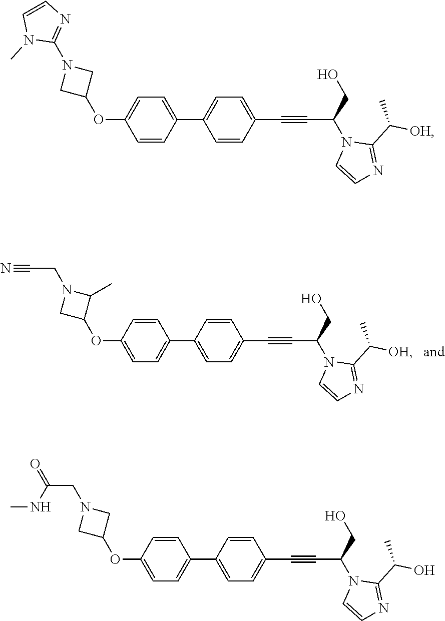 [SMILES: C[C@H](O)c1nccn1[C@@H](C#Cc1ccc(-c2ccc(OC3CN(c4nccn4C)C3)cc2)cc1)CO ; CC1C(Oc2ccc(-c3ccc(C#C[C@@H](CO)n4ccnc4[C@H](C)O)cc3)cc2)CN1CC#N ; "and" ; CNC(=O)CN1CC(Oc2ccc(-c3ccc(C#C[C@@H](CO)n4ccnc4[C@H](C)O)cc3)cc2)C1]
or a pharmaceutically acceptable salt, solvate, or stereoisomer thereof.

110. A compound selected from: 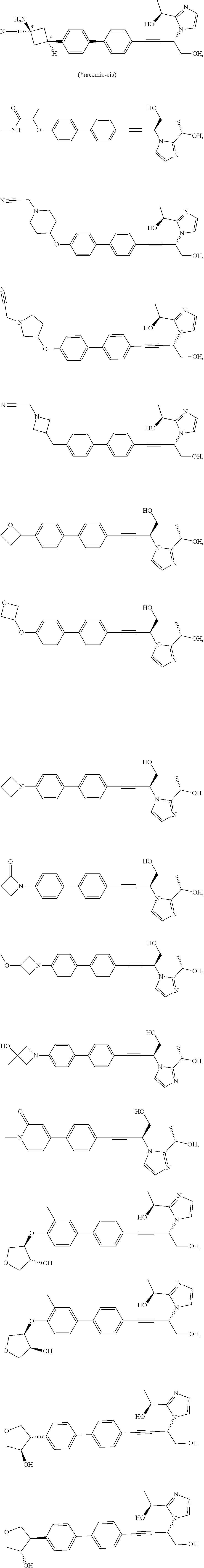 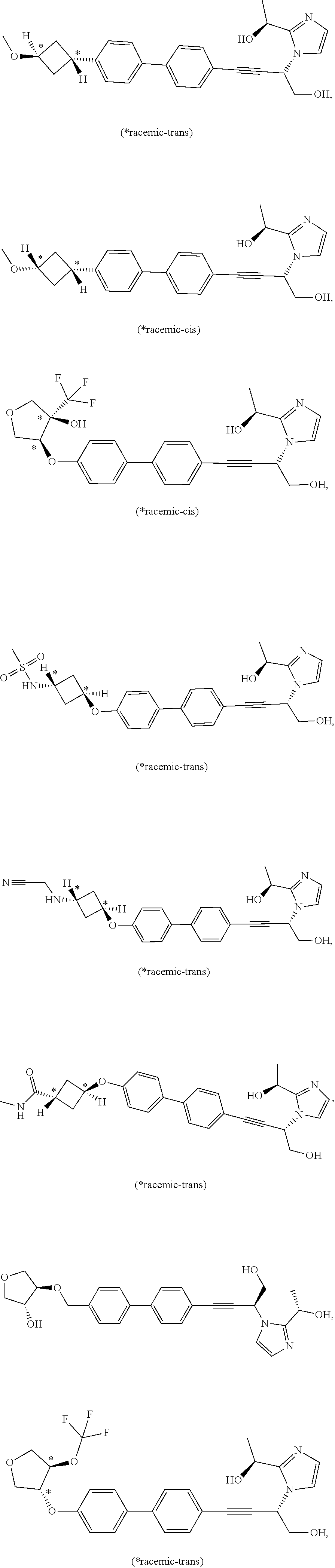 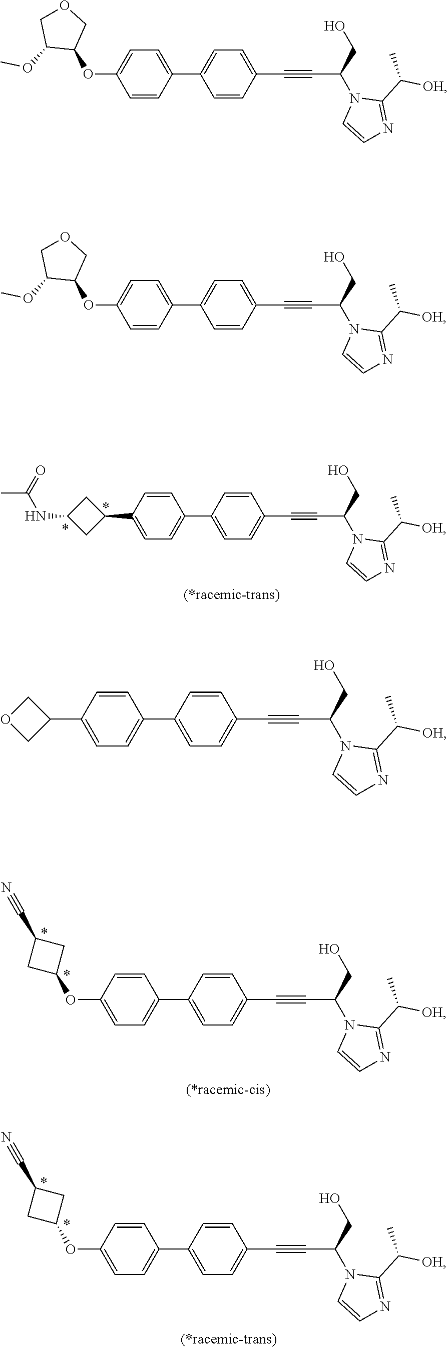 [SMILES: CO[C@@H]1COC[C@H]1Oc1ccc(-c2ccc(C#C[C@@H](CO)n3ccnc3[C@H](C)O)cc2)cc1 ; CO[C@@H]1COC[C@H]1Oc1ccc(-c2ccc(C#C[C@@H](CO)n3ccnc3[C@H](C)O)cc2)cc1 ; CC(=O)N[C@H]1C[C@H](c2ccc(-c3ccc(C#C[C@@H](CO)n4ccnc4[C@H](C)O)cc3)cc2)C1 ; C[C@H](O)c1nccn1[C@@H](C#Cc1ccc(-c2ccc(C3COC3)cc2)cc1)CO ; C[C@H](O)c1nccn1[C@@H](C#Cc1ccc(-c2ccc(O[C@H]3C[C@@H](C#N)C3)cc2)cc1)CO ; C[C@H](O)c1nccn1[C@@H](C#Cc1ccc(-c2ccc(O[C@H]3C[C@H](C#N)C3)cc2)cc1)CO] 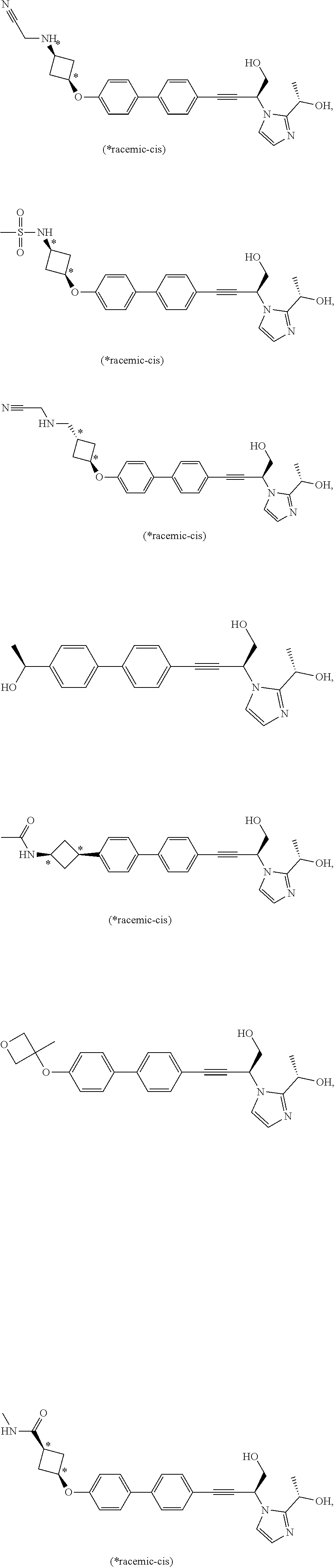 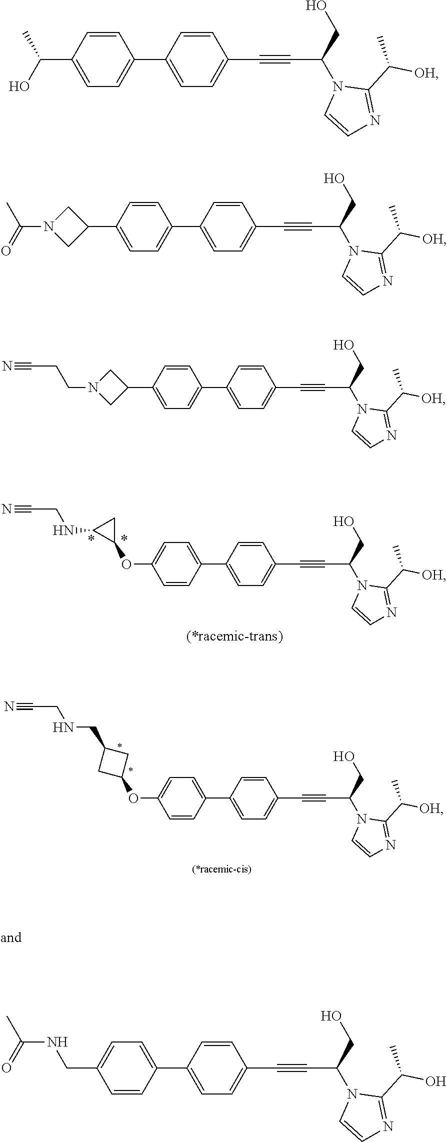
or a pharmaceutically acceptable salt, solvate, or stereoisomer thereof.

111. A pharmaceutical composition comprising the compound of any one of claims 1-110, or a pharmaceutically acceptable salt, solvate, or stereoisomer thereof, and a pharmaceutically acceptable excipient.

112. A method of treating or preventing a gram-negative bacterial infection in a patient in need thereof comprising administering to the patient the compound of any one of claims 1-110, or a pharmaceutically acceptable salt, solvate, or stereoisomer thereof, or the pharmaceutical composition of claim 111.

113. The method of claim 112, wherein the gram-negative bacterial infection is associated with Pseudomonas aeruginosa.

114. The method of claim 112, wherein the gram-negative bacterial infection is a respiratory infection.

115. The method of claim 114, wherein the respiratory infection is pneumonia.

116. The method of claim 115, wherein the pneumonia is community-acquired pneumonia (CAP), health care-associated pneumonia (HCAP), hospital-acquired pneumonia (HAP), ventilator-associate pneumonia (VAP), or a combination thereof.

117. A method of treating or preventing a P. aeruginosa infection in a patient in need thereof comprising administering to the patient the compound of any one of claims 1-110, or a pharmaceutically acceptable salt, solvate, or stereoisomer thereof, or the pharmaceutical composition of claim 111.

118. The method of any one of claims 112-117, wherein the patient has been identified as having a lung disease.

119. The method of claim 118, wherein the lung disease is a structural lung disease.

120. The method of claim 118 or claim 119, wherein the lung disease is cystic fibrosis, bronchiectasis, emphysema, chronic obstructive pulmonary disease (COPD), chronic destroyed lung disease, or a combination thereof.

121. The method of any one of claims 112-120, wherein the administration is to treat an existing infection.

122. The method of any one of claims 112-120, wherein the administration is provided as prophylaxis.

123. The method of any one of claims 112-122, wherein the compound of any one of claims 1-110, or a pharmaceutically acceptable salt, solvate, or stereoisomer thereof, or the pharmaceutical composition of claim 111, is administered in a solution by inhalation, intravenous injection, or intraperitoneal injection.

124. A compound of any one of claims 1-110 for use as therapeutically active substance.

125. A compound of any one of claims 1-110 for use in treating or preventing a gram-negative bacterial infection.

126. The compound for use of claim 125, wherein the gram-negative bacterial infection is associated with Pseudomonas aeruginosa.

127. The compound for use of claim 125, wherein the gram-negative bacterial infection is a respiratory infection.

128. The compound for use of claim 127, wherein the respiratory infection is pneumonia.

129. The compound for use of claim 128, wherein the pneumonia is community-acquired pneumonia (CAP), health care-associated pneumonia (HCAP), hospital-acquired pneumonia (HAP), ventilator-associate pneumonia (VAP), or a combination thereof.

130. A compound of any one of claims 1-110 for use in treating or preventing a P. aeruginosa infection.

131. The compound for use of any one of claims 124-130, wherein the patient has been identified as having a lung disease.

132. The compound for use of claim 131, wherein the lung disease is a structural lung disease.

133. The compound for use of claim 131 or claim 132, wherein the lung disease is cystic fibrosis, bronchiectasis, emphysema, chronic obstructive pulmonary disease (COPD), chronic destroyed lung disease, or a combination thereof.

134. The use of a compound of any one of claims 1-110 for the preparation of a medicament for treating or preventing a gram-negative bacterial infection.

135. The use of a compound of any one of claims 1-110 for treating or preventing a gram-negative bacterial infection.
